(19) United States
(12) Patent Application Publication
(10) Pub. No.: US 2003/0065156 A1
(43) Pub. Date: Apr. 3, 2003

(54) Novel human genes and gene expression products I

(76) Inventors: Lewis T. Williams, Mill Valley, CA (US); Jaime Escobedo, Alamo, CA (US); Michael A. Innis, Moraga, CA (US); Pablo Dominguez Garcia, San Francisco, CA (US); Julie Sudduth-Klinger, Kensington, CA (US); Christoph Reinhard, Alameda, CA (US); Klause Giese, San Francisco, CA (US); Filippo Randazzo, Emeryville, CA (US); Giulia C. Kennedy, San Francisco, CA (US); David Pot, San Francisco, CA (US); Atlaf Kassam, Oakland, CA (US); George Lamson, Moraga, CA (US); Radoje Drmanac, Palo Alto, CA (US); Radomir Crkvenjakov, Sunnyvale, CA (US); Mark Dickson, Hollister, CA (US); Snezana Drmanac, Palo Alto, CA (US); Ivan Labat, Sunnyvale, CA (US); Dena Leshkowitz, Sunnyvale, CA (US); David Kita, Foster City, CA (US); Veronica Garcia, Sunnyvale, CA (US); Lee William Jones, Sunnyvale, CA (US); Birgit Stache-Crain, Sunnyvale, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/076,555

(22) Filed: Feb. 15, 2002

Related U.S. Application Data
(63) Continuation of application No. 09/217,471, filed on Dec. 21, 1998.
(60) Provisional application No. 60/068,755, filed on Dec. 23, 1997.
(60) Provisional application No. 60/080,664, filed on Apr. 3, 1998.
(60) Provisional application No. 60/105,234, filed on Oct. 21, 1998.

(51) Int. Cl. C12Q 1/68; G01N 33/53; C07H 21/04

(52) U.S. Cl. 536/23.1; 435/6; 435/7.1

(57) ABSTRACT

This invention relates to novel human polynucleotides and variants thereof, their encoded polypeptides and variants thereof, to genes corresponding to these polynucleotides and to proteins expressed by the genes. The invention also relates to diagnostic and therapeutic agents employing such novel human polymucleotides, their corresponding genes or gene products, e.g., these genes and proteins, including probes, antisense constructs, and antibodies.

### CROSS-REFERENCES TO RELATED APPLICATIONS

This application is a continuation-in-part of U.S. provisional patent application serial No. 60/068,755, filed Dec. 23, 1997, and of U.S. provisional patent application serial No. 60/080,664, filed Apr. 3, 1998, and of U.S. provisional patent application serial No. 60/105,234, filed Oct. 21, 1998, each of which applications are incorporated herein by reference.

### FIELD OF THE INVENTION

The present invention relates to novel polynucleotides, particularly to novel polynucleotides of human origin that are expressed in a selected cell type, are differentially expressed in one cell type relative to another cell type (e.g., in cancerous cells, or in cells of a specific tissue origin) and/or share homology to polynucleotides encoding a gene product having an identified functional domain and/or activity.

### BACKGROUND OF THE INVENTION

Identification of novel polynucleotides, particularly those that encode an expressed gene product, is important in the advancement of drug discovery, diagnostic technologies, and the understanding of the progression and nature of complex diseases such as cancer. Identification of genes expressed in different cell types isolated from sources that differ in disease state or stage, developmental stage, exposure to various environmental factors, the tissue of origin, the species from which the tissue was isolated, and the like is key to identifying the genetic factors that are responsible for the phenotypes associated with these various differences This invention provides novel human polynucleotides, the polypeptides encoded by these polynucleotides, and the genes and proteins corresponding to these novel polynucleotides.

### SUMMARY OF THE INVENTION

This invention relates to novel human polynucleotides and variants thereof, their encoded polypeptides and variants thereof, to genes corresponding to these polynucleotides and to proteins expressed by the genes. The invention also relates to diagnostic and therapeutic agents employing such novel human polynucleotides, their corresponding genes or gene products, e.g., these genes and proteins, including probes, antisense constructs, and antibodies.

Accordingly, in one embodiment, the present invention features a library of polynucleotides, the library comprising the sequence information of at least one of SEQ ID NOS:1-844. In related aspects, the invention features a library provided on a nucleic acid array, or in a computer-readable format.

In one embodiment, the library is comprises a differentially expressed polynucleotide comprising a sequence selected from the group consisting of SEQ ID NOS:9, 39, 42, 52, 62, 74, 119, 172, 317, and 379. In specific related embodiments, the library comprises: 1) a polynucleotide that is differentially expressed in a human breast cancer cell, where the polynucleotide comprises a sequence selected from the group consisting of SEQ ID NOS:4, 9, 39, 42, 52, 62, 65, 66, 68, 74, 81, 114, 123, 144, 130, 157, 162, 172, 178, 183, 202, 214, 219, 223, 258, 298, 317, 338, 379, 384, 386, and 388; 2) a polynucleotide differentially expressed in a human colon cancer cell, where the polynucleotide comprises a sequence selected from the group consisting of SEQ ID NOS: 1, 39, 52, 97, 119, 134, 172, 176, 241, 288, 317, 357, 362, and 374; or 3) a polynucleotide differentially expressed in a human lung cancer cell, where the polynucleotide comprises a sequence selected from the group consisting of SEQ ID NOS: 9, 34, 42, 62, 74, 106, 119, 135, 154, 160, 260, 308, 323, 349, 361, 369, 371, 379, 395, 381, and 400.

In another aspect, the invention features an isolated polynucleotide comprising a nucleotide sequence having at least 90% sequence identity to an identifying sequence of SEQ ID NOS:1-844 or a degenerate variant thereof. In related aspects, the invention features recombinant host cells and vectors comprising the polynucleotides of the invention, as well as isolated polypeptides encoded by the polynucleotides of the invention and antibodies that specifically bind such polypeptides.

In one embodiment, the invention features an isolated polynucleotide comprising a sequence encoding a polypeptide of a protein family selected from the group consisting of: 4 transmembrane segments integral membrane proteins, 7 transmembrane receptors, ATPases associated with various cellular activities (AAA), eukaryotic aspartyl proteases, GATA family of transcription factors, G-protein alpha subunit, phorbol esters/diacylglycerol binding proteins, protein kinase, protein phosphatase 2C, protein tyrosine phosphatase, trypsin, wnt family of developmental signaling proteins, and WW/rsp5/WWP domain containing proteins. In a specific related embodiment, the invention features a polynucleotide comprising a sequence of one of SEQ ID NOS: 24, 41, 101, 157, 291, 305, 315, 341, 63, 116, 134, 136, 151, 384, 404, 308, 213, 367, 188, 251, 202, 315, 367, 397, 256, 382, 169, 23, 291, 324, 330, 341, 353, 188, 379, and 395.

In another embodiment, the invention features a polynucleotide comprising a sequence encoding a polypeptide having a functional domain selected from the group consisting of: Ank repeat, basic region plus leucine zipper transcription factors, bromodomain, EF-hand, SH3 domain, WD domain/G-beta repeats, zinc finger (C2H2 type), zinc finger (CCHC class), and zinc-binding metalloprotease domain. In a specific related embodiment, the invention features a polynucleotide comprising a sequence of one of SEQ ID NOS: 116, 251, 374, 97, 136, 242, 379, 306, 386, 18, 335, 61, 306, 386, 322, 306, and 395.

In another aspect, the invention features a method of detecting differentially expressed genes correlated with a cancerous state of a mammalian cell, where the method comprises the step of detecting at least one differentially expressed gene product in a test sample derived from a cell suspected of being cancerous, where the gene product is encoded by a gene corresponding to a sequence of at least one of SEQ ID NOS:4, 9, 39, 42, 52, 62, 65, 66, 68, 74, 81, 114, 123, 144, 130, 157, 162, 172, 178, 183, 202, 214, 219, 223, 258, 298, 317, 338, 379, 384, 386, 388, 1, 39, 52, 97, 119, 134, 172, 176, 241, 288, 317, 357, 362, 374, 9, 34, 42, 62, 74, 106, 119, 135, 154, 160, 260, 308, 323, 349, 361, 369, 371, 379, 395, 381, and 400. Detection of the differentially expressed gene product is correlated with a cancerous state of the cell from which the test sample was derived. In one embodiment, the detecting is by hybridization of the test sample to a reference array, wherein the reference array comprises an identifying sequence of at least one of SEQ ID NOS:1-844.

In one embodiment of the method of the invention, the cell is a breast tissue derived cell, and the differentially expressed gene product is encoded by a gene corresponding to a sequence of at least one of SEQ ID NOS: 4, 9, 39, 42, 52, 62, 65, 66, 68, 74, 81, 114, 123, 144, 130, 157, 162, 172, 178, 183, 202, 214, 219, 223, 258, 298, 317, 338, 379, 384, 386, and 388.

In another embodiment of the method of the invention, the cell is a colon tissue derived cell, and differentially expressed gene product is encoded by a gene corresponding to a sequence of at least one of SEQ ID NOS: 1, 39, 52, 97, 119, 134, 172, 176, 241, 288, 317, 357, 362, and 374.

In yet another embodiment of the method of the invention, the cell is a lung tissue derived cell, and differentially expressed gene product is encoded by a gene corresponding to a sequence of at least one of SEQ ID NOS: 9, 34, 42, 62, 74, 106, 119, 135, 154, 160, 260, 308, 323, 349, 361, 369, 371, 379, 395, 381, and 400.

Other aspects and embodiments of the invention will be readily apparent to the ordinarily skilled artisan upon reading the description provided herein.

### DETAILED DESCRIPTION OF THE INVENTION

The invention relates to polynucleotides comprising the disclosed nucleotide sequences, to full length cDNA, mRNA and genes corresponding to these sequences, and to polypeptides and proteins encoded by these polynucleotides and genes.

Also included are polynucleotides that encode polypeptides and proteins encoded by the polynucleotides of the Sequence Listing. The various polynucleotides that can encode these polypeptides and proteins differ because of the degeneracy of the genetic code, in that most amino acids are encoded by more than one triplet codon. The identity of such codons is well-known in this art, and this information can be used for the construction of the polynucleotides within the scope of the invention.

Polynucleotides encoding polypeptides and proteins that are variants of the polypeptides and proteins encoded by the polynucleotides and related cDNA and genes are also within the scope of the invention. The variants differ from wild type protein in having one or more amino acid substitutions that either enhance, add, or diminish a biological activity of the wild type protein. Once the amino acid change is selected, a polynucleotide encoding that variant is constructed according to the invention.

The following detailed description describes the polynucleotide compositions encompassed by the invention, methods for obtaining cDNA or genomic DNA encoding a full-length gene product, expression of these polynucleotides and genes, identification of structural motifs of the polynucleotides and genes, identification of the function of a gene product encoded by a gene corresponding to a polynucleotide of the invention, use of the provided polynucleotides as probes and in mapping and in tissue profiling, use of the corresponding polypeptides and other gene products to raise antibodies, and use of the polynucleotides and their encoded gene products for therapeutic and diagnostic purposes.

I. Polynucleotide Compositions

The scope of the invention with respect to polynucleotide compositions includes, but is not necessarily limited to, polynucleotides having a sequence set forth in any one of SEQ ID NOS:1-844; polynucleotides obtained from the biological materials described herein or other biological sources (particularly human sources) by hybridization under stringent conditions (particularly conditions of high stringency); genes corresponding to the provided polynucleotides; variants of the provided polynucleotides and their corresponding genes, particularly those variants that retain a biological activity of the encoded gene product (e.g., a biological activity ascribed to a gene product corresponding to the provided polynucleotides as a result of the assignment of the gene product to a protein family(ies) and/or identification of a functional domain present in the gene product). Other nucleic acid compositions contemplated by and within the scope of the present invention will be readily apparent to one of ordinary skill in the art when provided with the disclosure here.

The invention features polynucleotides that are expressed in cells of human tissue, specifically human colon, breast, and/or lung tissue. Novel nucleic acid compositions of the invention of particular interest comprise a sequence set forth in any one of SEQ ID NOS:1-844 or an identifying sequence thereof. An “identifying sequence” is a contiguous sequence of residues at least about 10 nt to about 20 nt in length, usually at least about 50 nt to about 100 nt in length, that uniquely identifies a polynucleotide sequence, e.g., exhibits less than 90%, usually less than about 80% to about 85% sequence identity to any contiguous nucleotide sequence of more than about 20 nt. Thus, the subject novel nucleic acid compositions include full length cDNAs or mRNAs that encompass an identifying sequence of contiguous nucleotides from any one of SEQ ID NOS:1-844.

The polynucleotides of the invention also include polynucleotides having sequence similarity or sequence identity. Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under stringent conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences (SEQ ID NOS:1-844) under stringent hybridization conditions. By using probes, particularly labeled probes of DNA sequences, one can isolate homologous or related genes. The source of homologous genes can be any species, e.g. primate species, particularly human; rodents, such as rats and mice, canines, felines, bovines, ovines, equines, yeast, nematodes, etc.

Preferably, hybridization is performed using at least 15 contiguous nucleotides of at least one of SEQ ID NOS: 1-844. That is, when at least 15 contiguous nucleotides of one of the disclosed SEQ ID NOs. is used as a probe, the probe will preferentially hybridize with a gene or mRNA (of the biological material) comprising the complementary sequence, allowing the identification and retrieval of the nucleic acids of the biological material that uniquely hybridize to the selected probe. Probes from more than one SEQ ID NO. will hybridize with the same gene or mRNA if the cDNA from which they were derived corresponds to one mRNA. Probes of more than 15 nucleotides can be used, but 15 nucleotides represents enough sequence for unique identification.

The polynucleotides of the invention also include naturally occurring variants of the nucleotide sequences (e.g., degenerate variants, allelic variants, etc.). Variants of the polynucleotides of the invention are identified by hybridization of putative variants with nucleotide sequences disclosed herein, preferably by hybridization under stringent conditions For example, by using appropriate wash conditions, variants of the polynucleotides of the invention can be identified where the allelic variant exhibits at most about 25-30% base pair mismatches relative to the selected polynucleotide probe. In general, allelic variants contain 15-25% base pair mismatches, and can contain as little as even 5-15%, or 2-5%, or 1-2% base pair mismatches, as well as a single base-pair mismatch.

The invention also encompasses homologs corresponding to the polynucleotides of SEQ ID NOS:1-844, where the source of homologous genes can be any mammalian species, e.g., primate species, particularly human; rodents, such as rats, canines, felines, bovines, ovines, equines, yeast, nematodes, etc. Between mammalian species, e.g., human and mouse, homologs have substantial sequence similarity, e.g., at least 75% sequence identity, usually at least 90%, more usually at least 95% between nucleotide sequences. Sequence similarity is calculated based on a reference sequence, which may be a subset of a larger sequence, such as a conserved motif, coding region, flanking region, etc. A reference sequence will usually be at least about 18 contiguous nt long, more usually at least about 30 nt long, and may extend to the complete sequence that is being compared. Algorithms for sequence analysis are known in the art, such as BLAST, described in Altschul et al., J. Mol. Biol. (1990) 215:403-10.

In general, variants of the invention have a sequence identity greater than at least about 65%, preferably at least about 75%, more preferably at least about 85%, and can be greater than at least about 90% or more as determined by the Smith-Waterman homology search algorithm as implemented in MPSRCH program (Oxford Molecular). For the purposes of this invention, a preferred method of calculating percent identity is the Smith-Waterman algorithm, using the following. Global DNA sequence identity must be greater than 65% as determined by the Smith-Waterman homology search algorithm as implemented in MPSRCH program (Oxford Molecular) using an affine gap search with the following search parameters: gap open penalty, 12; and gap extension penalty, 1.

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.). The term “cDNA” as used herein is intended to include all nucleic acids that share the arrangement of sequence elements found in native mature mRNA species, where sequence elements are exons and 3 and 5 non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3 and 5 untranslated regions found in the mature mRNA. It can further include specific transcriptional and translational regulatory sequences, such as promoters, enhancers, etc., including about 1 kb, but possibly more, of flanking genomic DNA at either the 5 and 3 end of the transcribed region. The genomic DNA can be isolated as a fragment of 100 kbp or smaller; and substantially free of flanking chromosomal sequence. The genomic DNA flanking the coding region, either 3 and 5, or internal regulatory sequences as sometimes found in introns, contains sequences required for proper tissue, stage-specific, or disease-state specific expression.

The nucleic acid compositions of the subject invention can encode all or a part of the subject differentially expressed polypeptides. Double or single stranded fragments can be obtained from the DNA sequence by chemically synthesizing oligonucleotides in accordance with conventional methods, by restriction enzyme digestion, by PCR amplification, etc. Isolated polynucleotides and polynucleotide fragments of the invention comprise at least about 10, about 15, about 20, about 35, about 50, about 100, about 150 to about 200, about 250 to about 300, or about 350 contiguous nucleotides selected from the polynucleotide sequences as shown in SEQ ID NOS:1-844. For the most part, fragments will be of at least 15 nt, usually at least 18 nt or 25 nt, and up to at least about 50 contiguous nt in length or more. In a preferred embodiment, the polynucleotide molecules comprise a contiguous sequence of at least twelve nucleotides selected from the group consisting of the polynucleotides shown in SEQ ID NOS:1-844.

Probes specific to the polynucleotides of the invention can be generated using the polynucleotide sequences disclosed in SEQ ID NOS:1-844. The probes are preferably at least about 12, 15, 16, 18, 20, 22, 24, or 25 nucleotide fragment of a corresponding contiguous sequence of SEQ ID NOS:1-844, and can be less than 2, 1, 0.5, 0.1, or 0.05 kb in length. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of a polynucleotide of one of SEQ ID NOS:1-844. More preferably, probes are designed based on a contiguous sequence of one of the subject polynucleotides that remain unmasked following application of a masking program for masking low complexity (e.g, XBLAST) to the sequence., i.e., one would select an unmasked region, as indicated by the polynucleotides outside the poly-n stretches of the masked sequence produced by the masking program.

The polynucleotides of the subject invention are isolated and obtained in substantial purity, generally as other than an intact chromosome. Usually, the polynucleotides, either as DNA or RNA, will be obtained substantially free of other naturally-occurring nucleic acid sequences, generally being at least about 50%, usually at least about 90% pure and are typically “recombinant”, e.g., flanked by one or more nucleotides with which it is not normally associated on a naturally occurring chromosome.

The polynucleotides of the invention can be provided as a linear molecule or within a circular molecule. They can be provided within autonomously replicating molecules (vectors) or within molecules without replication sequences. They can be regulated by their own or by other regulatory sequences, as is known in the art. The polynucleotides of the invention can be introduced into suitable host cells using a variety of techniques which are available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

The subject nucleic acid compositions can be used to, for example, produce polypeptides, as probes for the detection of mRNA of the invention in biological samples (e.g., extracts of human cells) to generate additional copies of the polynucleotides, to generate ribozymes or antisense oligonucleotides, and as single stranded DNA probes or as triple-strand forming oligonucleotides. The probes described herein can be used to, for example, determine the presence or absence of the polynucleotide sequences as shown in SEQ ID NOS:1-844 or variants thereof in a sample. These and other uses are described in more detail below.

Use of Polynucleotides to Obtain Full-Length cDNA and Full-Length Human Gene and Promoter Region

Full-length cDNA molecules comprising the disclosed polynucleotides are obtained as follows. A polynucleotide having a sequence of one of SEQ ID NOS:1-844, or a portion thereof comprising at least 12, 15, 18, or 20 nucleotides, is used as a hybridization probe to detect hybridizing members of a cDNA library using probe design methods, cloning methods, and clone selection techniques such as those described in U.S. Pat. No. 5,654,173. Libraries of cDNA are made from selected tissues, such as normal or tumor tissue, or from tissues of a mammal treated with, for example, a pharmaceutical agent. Preferably, the tissue is the same as the tissue from which the polynucleotides of the invention were isolated, as both the polynucleotides described herein and the cDNA represent expressed genes. Most preferably, the cDNA library is made from the biological material described herein in the Examples. Alternatively, many cDNA libraries are available commercially. (Sambrook et al., Molecular Cloning: A Laboratory Manual, 2nd Ed., (1989) Cold Spring Harbor Press, Cold Spring Harbor, N.Y.). The choice of cell type for library construction can be made after the identity of the protein encoded by the gene corresponding to the polynucleotide of the invention is known. This will indicate which tissue and cell types are likely to express the related gene, and thus represent a suitable source for the mRNA for generating the cDNA. Where the provided polynucleotides are isolated from cDNA libraries, the libraries are prepared from mRNA of human colon cells, more preferably, human colon cancer cells, even more preferably, from a highly metastatic colon cell, Km12L4-A.

Techniques for producing and probing nucleic acid sequence libraries are described, for example, in Sambrook et al., Molecular Cloning: A Laboratory Manual, 2nd Ed., (1989) Cold Spring Harbor Press, Cold Spring Harbor, N.Y. The cDNA can be prepared by using primers based on sequence from SEQ ID NOS:1-844. In one embodiment, the cDNA library can be made from only poly-adenylated mRNA. Thus, poly-T primers can be used to prepare cDNA from the mRNA.

Members of the library that are larger than the provided polynucleotides, and preferably that encompass the complete coding sequence of the native message, are obtained. In order to confirm that the entire cDNA has been obtained, RNA protection experiments are performed as follows. Hybridization of a full-length cDNA to an mRNA will protect the RNA from RNase degradation. If the cDNA is not full length, then the portions of the mRNA that are not hybridized will be subject to RNase degradation. This is assayed, as is known in the art, by changes in electrophoretic mobility on polyacrylamide gels, or by detection of released monoribonucleotides. Sambrook et al., Molecular Cloning: A Laboratory Manual, 2nd Ed., (1989) Cold Spring Harbor Press, Cold Spring Harbor, N.Y. In order to obtain additional sequences 5′ to the end of a partial cDNA, 5′ RACE (PCR Protocols: A Guide to Methods and Applications, (1990) Academic Press, Inc.) is performed.

Genomic DNA is isolated using the provided polynucleotides in a manner similar to the isolation of full-length cDNAs. Briefly, the provided polynucleotides, or portions thereof, are used as probes to libraries of genomic DNA. Preferably, the library is obtained from the cell type that was used to generate the polynucleotides of the invention, but this is not essential. Most preferably, the genomic DNA is obtained from the biological material described herein in the Examples. Such libraries can be in vectors suitable for carrying large segments of a genome, such as P1 or YAC, as described in detail in Sambrook et al., 9.4-9.30. In addition, genomic sequences can be isolated from human BAC libraries, which are commercially available from Research Genetics, Inc., Huntville, Ala., USA, for example. In order to obtain additional 5′ or 3′ sequences, chromosome walking is performed, as described in Sambrook et al., such that adjacent and overlapping fragments of genomic DNA are isolated. These are mapped and pieced together, as is known in the art, using restriction digestion enzymes and DNA ligase.

Using the polynucleotide sequences of the invention, corresponding full-length genes can be isolated using both classical and PCR methods to construct and probe cDNA libraries. Using either method, Northern blots, preferably, are performed on a number of cell types to determine which cell lines express the gene of interest at the highest level. Classical methods of constructing cDNA libraries are taught in Sambrook et al., supra. With these methods, cDNA can be produced from mRNA and inserted into viral or expression vectors. Typically, libraries of mRNA comprising poly(A) tails can be produced with poly(T) primers. Similarly, cDNA libraries can be produced using the instant sequences as primers.

PCR methods are used to amplify the members of a cDNA library that comprise the desired insert. In this case, the desired insert will contain sequence from the full length cDNA that corresponds to the instant polynucleotides. Such PCR methods include gene trapping and RACE methods. Gene trapping entails inserting a member of a cDNA library into a vector. The vector then is denatured to produce single stranded molecules. Next, a substrate-bound probe, such a biotinylated oligo, is used to trap cDNA inserts of interest. Biotinylated probes can be linked to an avidin-bound solid substrate. PCR methods can be used to amplify the trapped cDNA. To trap sequences corresponding to the full length genes, the labeled probe sequence is based on the polynucleotide sequences of the invention. Random primers or primers specific to the library vector can be used to amplify the trapped cDNA. Such gene trapping techniques are described in Gruber et al., WO 95/04745 and Gruber et al., U.S. Pat. No. 5,500,356. Kits are commercially available to perform gene trapping experiments from, for example, Life Technologies, Gaithersburg, Md., USA.

“Rapid amplification of cDNA ends,” or RACE, is a PCR method of amplifying cDNAs from a number of different RNAs. The cDNAs are ligated to an oligonucleotide linker, and amplified by PCR using two primers. One primer is based on sequence from the instant polynucleotides, for which full length sequence is desired, and a second primer comprises sequence that hybridizes to the oligonucleotide linker to amplify the cDNA. A description of this methods is reported in WO 97/19110. In preferred embodiments of RACE, a common primer is designed to anneal to an arbitrary adaptor sequence ligated to cDNA ends (Apte and Siebert, Biotechniques (1993) 15:890-893; Edwards et al., Nuc. Acids Res. (1991) 19:5227-5232). When a single gene-specific RACE primer is paired with the common primer, preferential amplification of sequences between the single gene specific primer and the common primer occurs. Commercial cDNA pools modified for use in RACE are available.

Another PCR-based method generates full-length cDNA library with anchored ends without needing specific knowledge of the cDNA sequence. The method uses lock-docking primers (I-VI), where one primer, poly TV (I-III) locks over the polyA tail of eukaryotic mRNA producing first strand synthesis and a second primer, polyGH (IV-VI) locks onto the polyC tail added by terminal deoxynucleotidyl transferase (TdT). This method is described in WO 96/40998.

The promoter region of a gene generally is located 5′ to the initiation site for RNA polymerase II. Hundreds of promoter regions contain the “TATA” box, a sequence such as TATTA or TATAA, which is sensitive to mutations. The promoter region can be obtained by performing 5′ RACE using a primer from the coding region of the gene. Alternatively, the cDNA can be used as a probe for the genomic sequence, and the region 5′ to the coding region is identified by “walking up.” If the gene is highly expressed or differentially expressed, the promoter from the gene can be of use in a regulatory construct for a heterologous gene.

Once the full-length cDNA or gene is obtained, DNA encoding variants can be prepared by site-directed mutagenesis, described in detail in Sambrook et al., 15.3-15.63. The choice of codon or nucleotide to be replaced can be based on disclosure herein on optional changes in amino acids to achieve altered protein structure and/or function.

As an alternative method to obtaining DNA or RNA from a biological material, nucleic acid comprising nucleotides having the sequence of one or more polynucleotides of the invention can be synthesized. Thus, the invention encompasses nucleic acid molecules ranging in length from 15 nucleotides (corresponding to at least 15 contiguous nucleotides of one of SEQ ID NOS: 1-844) up to a maximum length suitable for one or more biological manipulations, including replication and expression, of the nucleic acid molecule. The invention includes but is not limited to (a) nucleic acid having the size of a full gene, and comprising at least one of SEQ ID NOS: 1-844; (b) the nucleic acid of (a) also comprising at least one additional gene, operably linked to permit expression of a fusion protein; (c) an expression vector comprising (a) or (b); (d) a plasmid comprising (a) or (b) and (e) a recombinant viral particle comprising (a) or (b). Once provided with the polynucleotides disclosed herein, construction or preparation of (a)-(e) are well within the skill in the art.

The sequence of a nucleic acid comprising at least 15 contiguous nucleotides of at least any one of SEQ ID NOS: 1-844, preferably the entire sequence of at least any one of SEQ ID NOS: 1-844, is not limited and can be any sequence of A, T, G, and/or C (for DNA) and A, U, G, and/or C (for RNA) or modified bases thereof, including inosine and pseudouridine. The choice of sequence will depend on the desired function and can be dictated by coding regions desired, the intron-like regions desired, and the regulatory regions desired. Where the entire sequence of any one of SEQ ID NOS: 1-844 is within the nucleic acid, the nucleic acid obtained is referred to herein as a polynucleotide comprising the sequence of any one of SEQ ID NOS: 1-844.

II. Expression of Polypeptide Encoded by Full-Length cDNA or Full-Length Gene

The provided polynucleotide (e.g., a polynucleotide having a sequence of one of SEQ ID NOS:1-844), the corresponding cDNA, or the full-length gene is used to express a partial or complete gene product.

Constructs of polynucleotides having sequences of SEQ ID NOS :1-844 can be generated synthetically. Alternatively, single-step assembly of a gene and entire plasmid from large numbers of oligodeoxyribonucleotides is described by, e.g., Stemmer et al., Gene (Amsterdam) (1995) 164(1):49-53. In this method, assembly PCR (the synthesis of long DNA sequences from large numbers of oligodeoxyribonucleotides (oligos)) is described. The method is derived from DNA shuffling (Stemmer, Nature (1994) 370:389-391), and does not rely on DNA ligase, but instead relies on DNA polymerase to build increasingly longer DNA fragments during the assembly process. For example, a 1.1-kb fragment containing the TEM-1 beta-lactamase-encoding gene (bla) can be assembled in a single reaction from a total of 56 oligos, each 40 nucleotides (nt) in length. The synthetic gene can be PCR amplified and cloned in a vector containing the tetracycline-resistance gene (Tc-R) as the sole selectable marker. Without relying on ampicillin (Ap) selection, 76% of the Tc-R colonies were Ap-R, making this approach a general method for the rapid and cost-effective synthesis of any gene.

Appropriate polynucleotide constructs are purified using standard recombinant DNA techniques as described in, for example, Sambrook et al., Molecular Cloning: A Laboratory Manual, 2nd Ed., (1989) Cold Spring Harbor Press, Cold Spring Harbor, N.Y., and under current regulations described in United States Dept. of HHS, National Institute of Health (NIH) Guidelines for Recombinant DNA Research. The gene product encoded by a polynucleotide of the invention is expressed in any expression system, including, for example, bacterial, yeast, insect, amphibian and mammalian systems. Suitable vectors and host cells are described in U.S. Pat. No. 5,654,173.

Bacteria.

Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al., Proc. Natl. Acad. Sci. (USA) (1983) 80:21-25; and Siebenlist et al., Cell (1980) 20:269.

Yeast.

Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985) 25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990) 8:135; Kunze et al., J. Basic Microbiol. (1985) 25:141; Cregg et al., Mol. Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilburn et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

Insect Cells.

Expression of heterologous genes in insects is accomplished as described in U.S. Pat. No. 4,745,051; Friesen et al., “The Regulation of Baculovirus Gene Expression”, in: The Molecular Biology Of Baculoviruses (1986) (W. Doerfler, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Maeda et al., Nature (1985) 315:592-594.

Mammalian Cells.

Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. RE 30,985.

Polynucleotide molecules comprising a polynucleotide sequence provided herein propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. The partial or full-length polynucleotide is inserted into a vector typically by means of DNA ligase attachment to a cleaved restriction enzyme site in the vector. Alternatively, the desired nucleotide sequence can be inserted by homologous recombination in vivo. Typically this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

The polynucleotides set forth in SEQ ID NOS:1-844 or their corresponding full-length polynucleotides are linked to regulatory sequences as appropriate to obtain the desired expression properties. These can include promoters (attached either at the 5′ end of the sense strand or at the 3′ end of the antisense strand), enhancers, terminators, operators, repressors, and inducers. The promoters can be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art can be used.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Once the gene corresponding to a selected polynucleotide is identified, its expression can be regulated in the cell to which the gene is native. For example, an endogenous gene of a cell can be regulated by an exogenous regulatory sequence as disclosed in U.S. Pat. No. 5,641,670.

III. Identification of Functional and Structural Motifs of Novel Genes

A. Screening Polynucleotide Sequences and Amino Acid Sequences Against Publicly Available Databases

Translations of the nucleotide sequence of the provided polynucleotides, cDNAs or full genes can be aligned with individual known sequences. Similarity with individual sequences can be used to determine the activity of the polypeptides encoded by the polynucleotides of the invention. For example, sequences that show similarity with a chemokine sequence can exhibit chemokine activities. Also, sequences exhibiting similarity with more than one individual sequence can exhibit activities that are characteristic of either or both individual sequences.

The full length sequences and fragments of the polynucleotide sequences of the nearest neighbors can be used as probes and primers to identify and isolate the full length sequence corresponding to provided polynucleotides. The nearest neighbors can indicate a tissue or cell type to be used to construct a library for the full-length sequences corresponding to the provided polynucleotides.

Typically, a selected polynucleotide is translated in all six frames to determine the best alignment with the individual sequences. The sequences disclosed herein in the Sequence Listing are in a 5′ to 3′ orientation and translation in three frames can be sufficient (with a few specific exceptions as described in the Examples). These amino acid sequences are referred to, generally, as query sequences, which will be aligned with the individual sequences. Databases with individual sequences are described in “Computer Methods for Macromolecular Sequence Analysis” Methods in Enzymology (1996) 266, Doolittle, Academic Press, Inc., a division of Harcourt Brace & Co., San Diego, Calif., USA. Databases include Genbank, EMBL, and DNA Database of Japan (DDBJ).

Query and individual sequences can be aligned using the methods and computer programs described above, and include BLAST, available over the world wide web at http://ww.ncbi.nlm.nih.gov/BLAST/. Another alignment algorithm is Fasta, available in the Genetics Computing Group (GCG) package, Madison, Wis., USA, a wholly owned subsidiary of Oxford Molecular Group, Inc. Other techniques for alignment are described in Doolittle, supra. Preferably, an alignment program that permits gaps in the sequence is utilized to align the sequences. The Smith-Waterman is one type of algorithm that permits gaps in sequence alignments. See Meth. Mol. Biol. (1997) 70: 173-187. Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to identify sequences that are distantly related matches, and is especially tolerant of small gaps and nucleotide sequence errors. Amino acid sequences encoded by the provided polynucleotides can be used to search both protein and DNA databases.

Results of individual and query sequence alignments can be divided into three categories, high similarity, weak similarity, and no similarity. Individual alignment results ranging from high similarity to weak similarity provide a basis for determining polypeptide activity and/or structure. Parameters for categorizing individual results include: percentage of the alignment region length where the strongest alignment is found, percent sequence identity, and p value.

The percentage of the alignment region length is calculated by counting the number of residues of the individual sequence found in the region of strongest alignment, e.g., contiguous region of the individual sequence that contains the greatest number of residues that are identical to the residues of the corresponding region of the aligned query sequence. This number is divided by the total residue length of the query sequence to calculate a percentage. For example, a query sequence of 20 amino acid residues might be aligned with a 20 amino acid region of an individual sequence. The individual sequence might be identical to amino acid residues 5, 9-15, and 17-19 of the query sequence. The region of strongest alignment is thus the region stretching from residue 9-19, an 11 amino acid stretch. The percentage of the alignment region length is: 11 (length of the region of strongest alignment) divided by (query sequence length) 20 or 55%.

Percent sequence identity is calculated by counting the number of amino acid matches between the query and individual sequence and dividing total number of matches by the number of residues of the individual sequences found in the region of strongest alignment. Thus, the percent identity in the example above would be 10 matches divided by 11 amino acids, or approximately, 90.9%.

P value is the probability that the alignment was produced by chance. For a single alignment, the p value can be calculated according to Karlin et al., Proc. Natl. Acad. Sci. (1990) 87:2264 and Karlin et al., Proc. Natl. Acad. Sci. (1993) 90. The p value of multiple alignments using the same query sequence can be calculated using an heuristic approach described in Altschul et al., Nat. Genet. (1994) 6:119. Alignment programs such as BLAST program can calculate the p value.

Another factor to consider for determining identity or similarity is the location of the similarity or identity. Strong local alignment can indicate similarity even if the length of alignment is short. Sequence identity scattered throughout the length of the query sequence also can indicate a similarity between the query and profile sequences. The boundaries of the region where the sequences align can be determined according to Doolittle, supra; BLAST or FAST programs; or by determining the area where sequence identity is highest.

High Similarity.

In general, in alignment results considered to be of high similarity, the percent of the alignment region length is typically at least about 55% of total length query sequence; more typically, at least about 58%; even more typically; at least about 60% of the total residue length of the query sequence. Usually, percent length of the alignment region can be as much as about 62%; more usually, as much as about 64%; even more usually, as much as about 66%. Further, for high similarity, the region of alignment, typically, exhibits at least about 75% of sequence identity; more typically, at least about 78%; even more typically; at least about 80% sequence identity. Usually, percent sequence identity can be as much as about 82%; more usually, as much as about 84%; even more usually, as much as about 86%.

The p value is used in conjunction with these methods. If high similarity is found, the query sequence is considered to have high similarity with a profile sequence when the p value is less than or equal to about 10⁻²; more usually; less than or equal to about 10⁻³; even more usually; less than or equal to about 10⁻⁴. More typically, the p value is no more than about 10⁻⁵; more typically; no more than or equal to about 10⁻¹⁰; even more typically; no more than or equal to about 10⁻¹⁵ for the query sequence to be considered high similarity.

Weak Similarity.

In general, where alignment results considered to be of weak similarity, there is no minimum percent length of the alignment region nor minimum length of alignment. A better showing of weak similarity is considered when the region of alignment is, typically, at least about 15 amino acid residues in length; more typically, at least about 20; even more typically; at least about 25 amino acid residues in length. Usually, length of the alignment region can be as much as about 30 amino acid residues; more usually, as much as about 40; even more usually, as much as about 60 amino acid residues. Further, for weak similarity, the region of alignment, typically, exhibits at least about 35% of sequence identity; more typically, at least about 40%; even more typically; at least about 45% sequence identity. Usually, percent sequence identity can be as much as about 50%; more usually, as much as about 55%; even more usually, as much as about 60%.

If low similarity is found, the query sequence is considered to have weak similarity with a profile sequence when the p value is usually less than or equal to about 10⁻²; more usually; less than or equal to about 10⁻³; even more usually; less than or equal to about 10⁻⁴. More typically, the p value is no more than about 10⁻⁵; more usually; no more than or equal to about 10⁻¹⁰; even more usually; no more than or equal to about 10⁻¹⁵ for the query sequence to be considered weak similarity.

Similarity Determined by Sequence Identity Alone.

Sequence identity alone can be used to determine similarity of a query sequence to an individual sequence and can indicate the activity of the sequence. Such an alignment, preferably, permits gaps to align sequences. Typically, the query sequence is related to the profile sequence if the sequence identity over the entire query sequence is at least about 15%; more typically, at least about 20%; even more typically, at least about 25%; even more typically, at least about 50%. Sequence identity alone as a measure of similarity is most useful when the query sequence is usually, at least 80 residues in length; more usually, 90 residues; even more usually, at least 95 amino acid residues in length. More typically, similarity can be concluded based on sequence identity alone when the query sequence is preferably 100 residues in length; more preferably, 120 residues in length; even more preferably, 150 amino acid residues in length.

Determining Activity from Alignments with Profile and Multiple Aligned Sequences.

Translations of the provided polynucleotides can be aligned with amino acid profiles that define either protein families or common motifs. Also, translations of the provided polynucleotides can be aligned to multiple sequence alignments (MSA) comprising the polypeptide sequences of members of protein families or motifs. Similarity or identity with profile sequences or MSAs can be used to determine the activity of the gene products (e.g., polypeptides) encoded by the provided polynucleotides or corresponding cDNA or genes. For example, sequences that show an identity or similarity with a chemokine profile or MSA can exhibit chemokine activities.

Profiles can designed manually by (1) creating an MSA, which is an alignment of the amino acid sequence of members that belong to the family and (2) constructing a statistical representation of the alignment. Such methods are described, for example, in Birney et al., Nucl. Acid Res. (1996) 24(14): 2730-2739. MSAs of some protein families and motifs are publicly available. For example, http://genome.wustl.edu/Pfam/ includes MSAs of 547 different families and motifs. These MSAs are described also in Sonnhammer et al., Proteins (1997) 28: 405-420. Other sources over the world wide web include the site at http://www.emblheidelberg.de/argos/ali/ali.htm1; alternatively, a message can be sent to ALI@EMBLHEIDELBERG.DE for the information. A brief description of these MSAs is reported in Pascarella et al., Prot. Eng. (1996) 9(3):249-25 1. Techniques for building profiles from MSAs are described in Sonnhammer et al., supra; Birney et al., supra; and “Computer Methods for Macromolecular Sequence Analysis,” Methods in Enzymology (1996) 266, Doolittle, Academic Press, Inc., a division of Harcourt Brace & Co., San Diego, Calif., USA.

Similarity between a query sequence and a protein family or motif can be determined by (a) comparing the query sequence against the profile and/or (b) aligning the query sequence with the members of the family or motif. Typically, a program such as Searchwise is used to compare the query sequence to the statistical representation of the multiple alignment, also known as a profile. The program is described in Birney et al., supra. Other techniques to compare the sequence and profile are described in Sonnhammer et al., supra and Doolittle, supra.

Next, methods described by Feng et al., J. Mol. Evol. (1987) 25:351 and Higgins et al., CABIOS (1989) 5:151 can be used align the query sequence with the members of a family or motif, also known as a MSA. Computer programs, such as PILEUP, can be used. See Feng et al., infra. In general, the following factors are used to determine if a similarity between a query sequence and a profile or MSA exists: (1) number of conserved residues found in the query sequence, (2) percentage of conserved residues found in the query sequence, (3) number of frameshifts, and (4) spacing between conserved residues.

Some alignment programs that both translate and align sequences can make any number of frameshifts when translating the nucleotide sequence to produce the best alignment. The fewer frameshifts needed to produce an alignment, the stronger the similarity or identity between the query and profile or MSAs. For example, a weak similarity resulting from no frameshifts can be a better indication of activity or structure of a query sequence, than a strong similarity resulting from two frameshifts. Preferably, three or fewer frameshifts are found in an alignment; more preferably two or fewer frameshifts; even more preferably, one or fewer frameshifts; even more preferably, no frameshifts are found in an alignment of query and profile or MSAs.

Conserved residues are those amino acids found at a particular position in all or some of the family or motif members. For example, most chemokines contain four conserved cysteines. Alternatively, a position is considered conserved if only a certain class of amino acids is found in a particular position in all or some of the family members. For example, the N-terminal position can contain a positively charged amino acid, such as lysine, arginine, or histidine.

Typically, a residue of a polypeptide is conserved when a class of amino acids or a single amino acid is found at a particular position in at least about 40% of all class members; more typically, at least about 50%; even more typically, at least about 60% of the members. Usually, a residue is conserved when a class or single amino acid is found in at least about 70% of the members of a family or motif; more usually, at least about 80%; even more usually, at least about 90%; even more usually, at least about 95%.

A residue is considered conserved when three unrelated amino acids are found at a particular position in the some or all of the members; more usually, two unrelated amino acids. These residues are conserved when the unrelated amino acids are found at particular positions in at least about 40% of all class member; more typically, at least about 50%; even more typically. at least about 60% of the members. Usually, a residue is conserved when a class or single amino acid is found in at least about 70% of the members of a family or motif, more usually, at least about 80%; even more usually, at least about 90%; even more usually, at least about 95%.

A query sequence has similarity to a profile or MSA when the query sequence comprises at least about 25% of the conserved residues of the profile or MSA; more usually, at least about 30%; even more usually; at least about 40%. Typically, the query sequence has a stronger similarity to a profile sequence or MSA when the query sequence comprises at least about 45% of the conserved residues of the profile or MSA; more typically, at least about 50%; even more typically; at least about 55%.

B. Screening Polynucleotide and Amino Acid Sequences Against Protein Profiles

The identify and function of the gene that correlates to a polynucleotide described herein can be determined by screening the polynucleotides or their corresponding amino acid sequences against profiles of protein families. Such profiles focus on common structural motifs among proteins of each family. Publicly available profiles are described above in Section IVA. Additional or alternative profiles are described below.

In comparing a novel polynucleotide with known sequences, several alignment tools are available. Examples include PileUp, which creates a multiple sequence alignment, and is described in Feng et al., J. Mol. Evol. (1987) 25:351. Another method, GAP, uses the alignment method of Needleman et al., J. Mol. Biol. (1970) 48:443. GAP is best suited for global alignment of sequences. A third method, BestFit, functions by inserting gaps to maximize the number of matches using the local homology algorithm of Smith et al., Adv. Appl. Math. (1981) 2:482. Exemplary protein profiles are provided below and in the examples.

Chemokines.

Chemokines are a family of proteins that have been implicated in lymphocyte trafficking, inflammatory diseases, angiogenesis, hematopoiesis, and viral infection. See, for example, Rollins, Blood (1997) 90(3):909-928, and Wells et al., J. Leuk. Biol. (1997) 61:545-550. U.S. Pat. No. 5,605,817 discloses DNA encoding a chemokine expressed in fetal spleen. U.S. Pat. No. 5,656,724 discloses chemokine-like proteins and methods of use. U.S. Pat. No. 5,602,008 discloses DNA encoding a chemokine expressed by liver.

Chemokine mutants are polypeptides having an amino acid sequence that possesses at least one amino acid substitution, addition, or deletion as compared to native chemokines. Fragments possess the same amino acid sequence of the native chemokines; mutants can lack the amino and/or carboxyl terminal sequences. Fusions are mutants, fragments, or native chemokines that also include amino and/or carboxyl terminal amino acid extensions.

The number or type of the amino acid changes is not critical, nor is the length or number of the amino acid deletions, or amino acid extensions that are incorporated in the chemokines as compared to the native chemokine amino acid sequences. A polynucleotide encoding one of these variant polypeptides will retain at least about 80% amino acid identity with at least one known chemokine. Preferably, these polypeptides will retain at least about 85% amino acid sequence identity, more preferably, at least about 90%; even more preferably, at least about 95%. In addition, the variants exhibit at least 80%; preferably about 90%; more preferably about 95% of at least one activity exhibited by a native chemokine, which includes immunological, biological, receptor binding, and signal transduction flunctions.

Assays for chemotaxis relating to neutrophils are described in Walz et al., Biochem. Biophys. Res. Commun. (1987) 149:755, Yoshimura et al., Proc. Natl. Acad. Sci. (USA) (1987) 84:9233, and Schroder et al., J. Immunol. (1987) 139:3474; to lymphocytes, Larsen et al., Science (1989) 243:1464, Carr et al., Proc. Natl. Acad. Sci. (USA) (1994) 91:3652; to tumor-infiltrating lymphocytes, Liao et al., J. Exp. Med (1995). 182:1301; to hematopoietic progenitors, Aiuti et al., J. Exp. Med. (1 997) 185:111; to monocytes, Valente et al., Biochem. (1988) 27:4162; and to natural killer cells, Loetscher et al., J. Immunol. (1996) 156:322, and Allavena et al., Eur. J. Immunol. (1994) 24:3233.

Assays for determining the biological activity of attracting eosinophils are described in Dahinden et al., J. Exp. Med. (1994) 179:751, Weber et al., J. Immunol. (1995) 154:4166, and Noso et al., Biochem. Biophys. Res. Commun. (1994) 200:1470; for attracting dendritic cells, Sozzani et al., J. Immunol. (1995) 155:3292; for attracting basophils, in Dahinden et al., J. Exp. Med. (1994) 1 79:751, Alam et al., J. Immunol. (1994) 152:1298, Alam et al., J. Exp. Med. (1992) 176:781; and for activating neutrophils, Maghazaci et al., Eur. J. Immunol. (1996) 26:315, and Taub et al., J. Immunol. (1995) 155:3877. Native chemokines can act as mitogens for fibroblasts, assayed as described in Mullenbach et al., J. Biol. Chem. (1986) 261:719.

Native chemokines exhibit binding activity with a number of receptors. Description of such receptors and assays to detect binding are described in, for example, Murphy et al., Science (1991) 253:1280; Combadiere et al., J. Biol. Chem. (1995) 270:29671; Daugherty et al., J. Exp. Med. (1996) 183:2349; Samson et al., Biochem. (1996) 35:3362; Raport et al., J. Biol. Chem. (1996) 271:17161; Combadiere et al., J. Leukoc. Biol. (1996) 60:147; Baba et al., J. Biol. Chem. (1997) 23:14893; Yosida et al., J. Biol. Chem. (1997) 272:13803; Arvannitakis et al., Nature (1997) 385:347, and other assays are known in the art.

Assays for kinase activation of chemokines are described by Yen et al., J. Leukoc. Biol. (1997) 61:529; Dubois et al., J. Immunol. (1996) 156:1356; Turner et al., J. Immunol. (1995) 155:2437. Assays for inhibition of angiogenesis or cell proliferation are described in Maione et al., Science (1990) 247:77. Glycosaminoglycan production can be induced by native chemokines, assayed as described in Castor et al., Proc. Natl. Acad. Sci. (USA) (1983) 80:765. Chemokine-mediated histamine release from basophils is assayed as described in Dahinden et al., J. Exp. Med. (1989) 170:1787; and White et al., Immunol. Lett. (1989) 22:151. Heparin binding is described in Luster et al., J. Exp. Med. (1995) 182:219.

Chemokines can possess dimerization activity, which can be assayed according to Burrows et al., Biochem. (1994)33:12741; and Zhang et al., Mol. Cell. Biol. (1995) 15:4851. Native chemokines can play a role in the inflammatory response of viruses. This activity can be assayed as described in Bleul et al., Nature (1996) 382:829; and Oberlin et al., Nature (1996) 382:833. Exocytosis of monocytes can be promoted by native chemokines. The assay for such activity is described in Uguccioni et al., Eur. J. Immunol. (1995) 25:64. Native chemokines also can inhibit hematopoietic stem cell proliferation. The method for testing for such activity is reported in Graham et al., Nature (1990) 344:442.

Death Domain Proteins.

Several protein families contain death domain motifs (Feinstein and Kimchi, TIBS Letters (1995) 20:242). Some death domain containing proteins are implicated in cytotoxic intracellular signaling (Cleveland et al., Cell (1995) 81:479, Pan et al, Science (1997) 276:111; Duan et al., Nature (1997) 385:86-89, and Chimlaiyan et al, Science (1996) 274:990). U.S. Pat. No. 5,563,039 describes a protein homologous to TRADD (Tumor Necrosis Factor Receptor-1 Associated Death Domain containing protein), and modifications of the active domain of TRADD that retain the functional characteristics of the protein, as well as apoptosis assays for testing the function of such death domain containing proteins. U.S. Pat. No. 5,658,883 discloses biologically active TGF-B1 peptides. U.S. Pat. No. 5,674,734 discloses RIP, which contains a C-terminal death domain and an N-terminal kinase domain.

Leukemia Inhibitory Factor (LIF).

An LIF profile is constructed from sequences of leukemia inhibitor factor, CT-1 (cardiotrophin-1), CNTF (ciliary neurotrophic factor), OSM (oncostatin M), and IL-6 (interleukin-6). This profile encompasses a family of secreted cytokines that have pleiotropic effects on many cell types including hepatocytes, osteoclasts, neuronal cells and cardiac myocytes, and can be used to detect additional genes encoding such proteins. These molecules are all structurally related and share a common co-receptor gpi 30 which mediates intracellular signal transduction by cytoplasmic tyrosine kinases such as src.

Novel proteins related to this family are also likely to be secreted, to activate gp 130 and to function in the development of a variety of cell types. Thus new members of this family would be candidates to be developed as growth or survival factors for the cell types that they stimulate. For more details on this family of cytokines, see Pennica et al, Cytokine and Growth Factor Reviews (1996) 7:81-91. U.S. Pat. No. 5,420,247 discloses LIF receptor and fusion proteins. U.S. Pat. No. 5,443,825 discloses human LIF.

Angiopoietin.

Angiopoietin-1 is a secreted ligand of the TIE-2 tyrosine kinase; it functions as an angiogenic factor critical for normal vascular development. Angiopoietin-2 is a natural antagonist of angiopoietin-1 and thus functions as an anti-angiogenic factor. These two proteins are structurally similar and activate the same receptor (Folkman et al., Cell (1996) 87:1153, and Davis et al., Cell (1996) 87:1161). The angiopoietin molecules are composed of two domains: a coiled-coil region and a region related to fibrinogen. The fibrinogen domain is found in many molecules including ficolin and tesascin, and is well defined structurally with many members.

Receptor Protein-Tyrosine Kinases.

Receptor Protein-Tyrosine Kinases or RPTKs are described in Lindberg, Annu. Rev. Cell Biol. (1994) 10:251-337.

Growth Factors: (Epidermal Growth Factor) EGF and (Fibroblast Growth Factor) FGF.

For a discussion of growth factor superfamilies, see Growth Factors: A Practical Approach, (Appendix A1) (1993) McKay and Leigh, Oxford University Press, NY, 237-243. U.S. Pat. No. 4,444,760 discloses acidic brain fibroblast growth factor, which is active in the promotion of cell division and wound healing. U.S. Pat. No. 5,439,818 discloses DNA encoding human recombinant basic fibroblast growth factor, which is active in wound healing. U.S. Pat. No. 5,604,293 discloses recombinant human basic fibroblast growth factor, which is useful for wound healing. U.S. Pat. No. 5,410,832 discloses brain-derived and recombinant acidic fibroblast growth factor, which act as mitogens for mesoderm and neuroectoderm-derived cells in culture, and promote wound healing in soft tissue, cartilaginous tissue and musculo-skeletal tissue. U.S. Pat. No. 5,387,673 discloses biologically active fragments of FGF.

Proteins of the TNF Family.

A profile derived from the TNF family is created by aligning sequences of the following TNF family members: nerve growth factor (NGF), lymphotoxin, Fas ligand, tumor necrosis factor (TNFα), CD40 ligand, TRAIL, ox40 ligand, 4-1BB ligand, CD27 ligand, and CD30 ligand. The profile is designed to identify sequences of proteins that constitute new members or homologues of this family of proteins. U.S. Pat. No. 5,606,023 discloses mutant TNF proteins; U.S. Pat. No. 5,597,899 and U.S. Pat. No. 5,486,463 disclose TNF muteins; and U.S. Pat. No. 5,652,353 discloses DNA encoding TNFα muteins.

Members of the TNF family of proteins have been show in vitro to multimerize, as described in Burrows et al., Biochem. (1994) 33:12741 and Zhang et al., Mol. Cell. Biol. (1995) 15:4851 and bind receptors as described in Browning et al., J. Immunol. (1994) 147:1230, Androlewicz et al., J. Biol. Chem.(1992) 267:2542, and Crowe et al., Science (1994) 264:707.

In vivo, TNFs proteolytically cleave a target protein as described in Kriegel et al., Cell (1988) 53:45 and Mohler et al., Nature (1994) 370:218 and demonstrate cell proliferation and differentiation activity. T-cell or thymocyte proliferation is assayed as described in Armitage et al., Eur. J. Immunol. (1992) 22:447; Current Protocols in Immunology, ed. J. E. Coligan et al., 3.1-3.19; Takai et al., J. Immunol. (1986)137:3494-3500, Bertagnoli et al., J. Immunol. (1990) 145:1706, Bertagnoli et al., J. Immunol. (1991) 133:327, Bertagnoli et al., J. Immunol. (1992) 149:3778, and Bowman et al., J. Immunol. (1994) 152:1756. B cell proliferation and Ig secretion are assayed as described in Maliszewski, J. Immunol. (1990) 144:3028, and Assays for B Cell Function: In Vitro Antibody Production, Mond and Brunswick, Current Protocols in Immunol., Coligan Ed vol 1 pp 3.8.1-3.8.16, John Wiley and Sons, Toronto 1994, Kehrl et al., Science (1987)238:1144 and Boussiotis et al., PNAS USA (1994) 91:7007. Other in vivo activities include upregulation of cell surface antigens, upregulation of costimulatory molecules, and cellular aggregation/adhesion as described in Barrett et al., J. Immunol. (1 991) 146:1722; Bjorck et al., Eur. J. Immunol. (i 993) 23:1771; Clark et al., Annu Rev. Immunol. (1 991) 9:97; Ranheim et al., J. Exp. Med. (1994) 177:925; Yellin, J. Immunol. (1994) 153:666; and Gruss et al., Blood (1994) 84:2305.

Proliferation and differentiation of hematopoietic and lymphopoietic cells has also been shown in vivo for TNFs, using assays for embryonic differentiation and hematopoiesis as described in Johansson et al., Cellular Biology (1995) 15:141, Keller et al., Mol. Cell. Biol. (1993) 13:473, McClanahan et al., Blood (1993) 81:2903 and using assays to detect stem cell survival and differentiation as described in Culture of Hematopoietic Cells, Freshney et al. eds, pp 1-21, 23-29, 139-162, 163-179, and 265-268, Wiley-Liss, Inc., New York, N.Y., 1994, and Hirajama et al., PNAS USA (1992) 89:5907.

In vivo activities of TNFs also include lymphocyte survival and apoptosis, assayed as described in Darzynkewicz et al., Cytometry (1992) 13:795; Gorczca et al., Leukemia (1993) 7:659; Itoh et al., Cell (1991) 66:233; Zacharduk, J. Immunol. (1990) 145:4037; Zamai et al., Cytometry (1993) 14:891; and Gorczyca et al., Int'l J. Oncol. (1992) 1:639. Some members of the TNF family are cleaved from the cell surface; others remain membrane bound. The three-dimensional structure of TNF is discussed in Sprang and Eck, Tumor Necrosis Factors; supra.

TNF proteins include a transmembrane domain. The protein is cleaved into a shorter soluble version, as described in Kriegler et al., Cell (1988) 53:45, Perez et al., Cell (1990) 63:251, and Shaw et al., Cell (1986) 46:659. The transmembrane domain is between amino acid 46 and 77 and the cytoplasmic domain is between position 1 and 45 on the human form of TNFα. The 3-dimensional motifs of TNF include a sandwich of two pleated β sheets. Each sheet is composed of anti-parallel β strands. β strands facing each other on opposite sites of the sandwich are connected by short polypeptide loops, as described in Van Ostade et al., Protein Engineering (1994) 7(1):5, and Sprang et al., Tumor Necrosis Factors; supra. Residues of the TNF family proteins that are involved in the β sheet secondary structure have been identified as described in Van Ostade et al., Protein Eng. (1994) 7(1):5, and Sprang et al., supra.

TNF receptors are disclosed in U.S. Pat. No. 5,395,760. A profile derived from the TNF receptor family is created by aligning sequences of the TNF receptor family, including Apo1/Fas, TNFR I and II, death receptor 3 (DR3), CD40, ox40, CD27, and CD30. Thus, the profile is designed to identify from the polynucleotides of the invention sequences of proteins that constitute new members or homologues of this family of proteins.

Tumor necrosis factor receptors exist in two forms in humans: p55 TNFR and p75 TNFR, both of which provide intracellular signals upon binding with a ligand. The extracellular domains of these receptor proteins are cysteine rich. The receptors can remain membrane bound, although some forms of the receptors are cleaved forming soluble receptors. The regulation, diagnostic, prognostic, and therapeutic value of soluble TNF receptors is discussed in Aderka, Cytokine and Growth Factor Reviews, (1996) 7(3):231.

PDGF Family.

U.S. Pat. No. 5,326,695 discloses platelet derived growth factor agonists; bioactive portions of PDGF-B are used as agonists. U.S. Pat. No. 4,845,075 discloses biologically active B-chain homodimers, and also includes variants and derivatives of the PDGF-B chain. U.S. Pat. No. 5,128,321 discloses PDGF analogs and methods of use. Proteins having the same bioactivity as PDGF are disclosed, including A and B chain proteins.

Kinase (Including MKK) Family.

U.S. Pat. No. 5,650,501 discloses serine/threonine kinase, associated with mitotic and meiotic cell division; the protein has a kinase domain in its N-terminal and 3 PEST regions in the C-terminus. U.S. Pat. No. 5,605,825 discloses human PAK65, a serine protein kinase.

The foregoing discussion provides a few examples of the protein profiles that can be compared with the polynucleotides of the invention. One skilled in the art can use these and other protein profiles to identify the genes that correlate with the provided polynucleotides.

C. Identification of Secreted & Membrane-Bound Polypeptides

Both secreted and membrane-bound polypeptides of the present invention are of particular interest. For example, levels of secreted polypeptides can be assayed in body fluids that are convenient, such as blood, urine, prostatic fluid and semen. Membrane-bound polypeptides are useful for constructing vaccine antigens or inducing an immune response. Such antigens would comprise all or part of the extracellular region of the membrane-bound polypeptides. Because both secreted and membrane-bound polypeptides comprise a fragment of contiguous hydrophobic amino acids, hydrophobicity predicting algorithms can be used to identify such polypeptides.

A signal sequence is usually encoded by both secreted and membrane-bound polypeptide genes to direct a polypeptide to the surface of the cell. The signal sequence usually comprises a stretch of hydrophobic residues. Such signal sequences can fold into helical structures. Membrane-bound polypeptides typically comprise at least one transmembrane region that possesses a stretch of hydrophobic amino acids that can transverse the membrane. Some transmembrane regions also exhibit a helical structure. Hydrophobic fragments within a polypeptide can be identified by using computer algorithms. Such algorithms include Hopp & Woods, Proc. Natl. Acad. Sci. USA (1981) 78:3824-3828; Kyte & Doolittle, J. Mol. Biol. (1982) 157: 105-132; and RAOAR algorithm, Degli Esposti et al., Eur. J. Biochem. (1990)190: 207-219.

Another method of identifying secreted and membrane-bound polypeptides is to translate the polynucleotides of the invention in all six frames and determine if at least 8 contiguous hydrophobic amino acids are present. Those translated polypeptides with at least 8; more typically, 10; even more typically, 12 contiguous hydrophobic amino acids are considered to be either a putative secreted or membrane bound polypeptide. Hydrophobic amino acids include alanine, glycine, histidine, isoleucine, leucine, lysine, methionine, phenylalanine, proline, threonine, tryptophan, tyrosine, and valine.

IV. Identification of the Function of an Expression Product of a Full-Length Gene Corresponding to a Polynucleotide

Ribozymes, antisense constructs, and dominant negative mutants can be used to determine function of the expression product of a gene corresponding to a polynucleotide provided herein. These methods and compositions are particularly useflul where the provided novel polynucleotide exhibits no significant or substantial homology to a sequence encoding a gene of known function. Antisense molecules and ribozymes can be constructed from synthetic polynucleotides. Typically, the phosphoramidite method of oligonucleotide synthesis is used. See Beaucage et al., Tet. Lett. (1981) 22:1859 and U.S. Pat. No. 4,668,777. Automated devices for synthesis are available to create oligonucleotides using this chemistry. Examples of such devices include Biosearch 8600, Models 392 and 394 by Applied Biosystems, a division of Perkin-Elmer Corp., Foster City, Calif., USA; and Expedite by Perceptive Biosystems, Framingham, Mass., USA. Synthetic RNA, phosphate analog oligonucleotides, and chemically derivatized oligonucleotides can also be produced, and can be covalently attached to other molecules. RNA oligonucleotides can be synthesized, for example, using RNA phosphoramidites. This method can be performed on an automated synthesizer, such as Applied Biosystems, Models 392 and 394, Foster City, Calif., USA. See Applied Biosystems User Bulletin 53 and Ogilvie et al., Pure & Applied Chem. (1987) 59:325.

Phosphorothioate oligonucleotides can also be synthesized for antisense construction. A sulfurizing reagent, such as tetraethylthiruam disulfide (TETD) in acetonitrile can be used to convert the internucleotide cyanoethyl phosphite to the phosphorothioate triester within 15 minutes at room temperature. TETD replaces the iodine reagent, while all other reagents used for standard phosphoramidite chemistry remain the same. Such a synthesis method can be automated using Models 392 and 394 by Applied Biosystems, for example.

Oligonucleotides of up to 200 nucleotides can be synthesized, more typically, 100 nucleotides, more typically 50 nucleotides; even more typically 30 to 40 nucleotides. These synthetic fragments can be annealed and ligated together to construct larger fragments. See, for example, Sambrook et al., supra.

A. Ribozymes

Trans-cleaving catalytic RNAs (ribozymes) are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting the phenotypic effect.

One commonly used ribozyme motif is the hammerhead, for which the substrate sequence requirements are minimal. Design of the hammerhead ribozyme is disclosed in Usman et al., Current Opin. Struct. Biol. (1996) 6:527. Usman also discusses the therapeutic uses of ribozymes. Ribozymes can also be prepared and used as described in Long et al., FASEB J. (1993) 7:25; Symons, Ann. Rev. Biochem. (1992) 61:641; Perrotta et al., Biochem. (1992) 31:16; Ojwang et al., Proc. Natl. Acad. Sci. (USA) (1992) 89:10802; and U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337 and Koizumi et al., Nucleic Acid Res. (1989) 17:7059. Preparation and use of ribozyme fragments in a hammerhead structure are also described by Koizumi et al., Nucleic Acids Res. (1989) 17:7059. Preparation and use of ribozyme fragments in a hairpin structure are described by Chowrira and Burke, Nucleic Acids Res. (1992) 20:2835. Ribozymes can also be made by rolling transcription as described in Daubendiek and Kool, Nat. Biotechnol. (1997) 15(3):273.

The hybridizing region of the ribozyme can be modified or can be prepared as a branched structure as described in Horn and Urdea, Nucleic Acids Res. (1989) 17:6959. The basic structure of the ribozymes can also be chemically altered in ways familiar to those skilled in the art, and chemically synthesized ribozymes can be administered as synthetic oligonucleotide derivatives modified by monomeric units. In a therapeutic context, liposome mediated delivery of ribozymes improves cellular uptake, as described in Birikh et al., Eur. J. Biochem. (1997) 245:1.

Using the polynucleotide sequences of the invention and methods known in the art, ribozymes are designed to specifically bind and cut the corresponding mRNA species. Ribozymes thus provide a means to inihibit the expression of any of the proteins encoded by the disclosed polynucleotides or their full-length genes. The full-length gene need not be known in order to design and use specific inhibitory ribozymes. In the case of a polynucleotide or full-length cDNA of unknown function, ribozymes corresponding to that nucleotide sequence can be tested in vitro for efficacy in cleaving the target transcript. Those ribozymes that effect cleavage in vitro are further tested in vivo. The ribozyme can also be used to generate an animal model for a disease, as described in Birikh et al., supra. An effective ribozyme is used to determine the function of the gene of interest by blocking its transcription and detecting a change in the cell. Where the gene is found to be a mediator in a disease, an effective ribozyme is designed and delivered in a gene therapy for blocking transcription and expression of the gene.

Therapeutic and functional genomic applications of ribozymes proceed beginning with knowledge of a portion of the coding sequence of the gene to be inhibited. Thus, for many genes, a partial polynucleotide sequence provides adequate sequence for constructing an effective ribozyme. A target cleavage site is selected in the target sequence, and a ribozyme is constructed based on the 5′ and 3′ nucleotide sequences that flank the cleavage site. Retroviral vectors are engineered to express monomeric and multimeric hammerhead ribozymes targeting the mRNA of the target coding sequence. These monomeric and multimeric ribozymes are tested in vitro for an ability to cleave the target mRNA. A cell line is stably transduced with the retroviral vectors expressing the ribozymes, and the transduction is confirmed by Northern blot analysis and reverse-transcription polymerase chain reaction (RT-PCR). The cells are screened for inactivation of the target mRNA by such indicators as reduction of expression of disease markers or reduction of the gene product of the target mRNA.

B. Antisense

Antisense nucleic acids are designed to specifically bind to RNA, resulting in the formation of RNA-DNA or RNA-RNA hybrids, with an arrest of DNA replication, reverse transcription or messenger RNA translation. Antisense polynucleotides based on a selected polynucleotide sequence can interfere with expression of the corresponding gene. Antisense polynucleotides are typically generated within the cell by expression from antisense constructs that contain the antisense strand as the transcribed strand. Antisense polynucleotides based on the disclosed polynucleotides will bind and/or interfere with the translation of mRNA comprising a sequence complementary to the antisense polynucleotide. The expression products of control cells and cells treated with the antisense construct are compared to detect the protein product of the gene corresponding to the polynucleotide upon which the antisense construct is based. The protein is isolated and identified using routine biochemical methods.

One rationale for using antisense methods to determine the function of the gene corresponding to a disclosed polynucleotide is the biological activity of antisense therapeutics. Antisense therapy for a variety of cancers is in clinical phase and has been discussed extensively in the literature. Reed reviewed antisense therapy directed at the Bcl-2 gene in tumors; gene transfer-mediated overexpression of Bcl-2 in tumor cell lines conferred resistance to many types of cancer drugs. (Reed, J. C., N.C.I. (1997) 89:988). The potential for clinical development of antisense inhibitors of ras is discussed by Cowsert, L. M., Anti-Cancer Drug Design (1997) 12:359. Additional important antisense targets include leukemia (Geurtz, A. M., Anti-Cancer Drug Design (1997) 12:341); human C-ref kinase (Monia, B. P., Anti-Cancer Drug Design (1997) 12:327); and protein kinase C (McGraw et al., Anti-Cancer Drug Design (1997) 12:315.

Given the extensive background literature and clinical experience in antisense therapy, one skilled in the art can use selected polynucleotides of the invention as additional potential therapeutics. The choice of polynucleotide can be narrowed by first testing them for binding to “hot spot” regions of the genome of cancerous cells. If a polynucleotide is identified as binding to a “hot spot”, testing the polynucleotide as an antisense compound in the corresponding cancer cells clearly is warranted.

Ogunbiyi et al., Gastroenterology (1997) 113(3):761 describe prognostic use of allelic loss in colon cancer; Barks et al., Genes, Chromosomes, and Cancer (1997) 19(4):278 describe increased chromosome copy number detected by FISH in malignant melanoma; Nishizake et al., Genes, Chromosomes, and Cancer (1997) 19(4):267 describe genetic alterations in primary breast cancer and their metastases and direct comparison using modified comparative genome hybridization; and Elo et al., Cancer Research (1997) 57(16):3356 disclose that loss of heterozygosity at 16z24.1-q24.2 is significantly associated with metastatic and aggressive behavior of prostate cancer.

C. Dominant Negative Mutations

As an alternative method for identifying function of the gene corresponding to a polynucleotide disclosed herein, dominant negative mutations are readily generated for corresponding proteins that are active as homomultimers. A mutant polypeptide will interact with wild-type polypeptides (made from the other allele) and form a non-functional multimer. Thus, a mutation is in a substrate-binding domain, a catalytic domain, or a cellular localization domain. Preferably, the mutant polypeptide will be overproduced. Point mutations are made that have such an effect. In addition, fusion of different polypeptides of various lengths to the terminus of a protein can yield dominant negative mutants. General strategies are available for making dominant negative mutants (see, e.g., Herskowitz, Nature (1987) 329:219). Such techniques can be used to create loss of function mutations, which are useful for determining protein function.

V. Construction of Polypeptides of the Invention and Variants Thereof

The polypeptides of the invention include those encoded by the disclosed polynucleotides. These polypeptides can also be encoded by nucleic acids that, by virtue of the degeneracy of the genetic code, are not identical in sequence to the disclosed polynucleotides. Thus, the invention includes within its scope a polypeptide encoded by a polynucleotide having the sequence of any one of SEQ ID NOS: 1-844 or a variant thereof.

In general, the term “polypeptide” as used herein refers to both the full length polypeptide encoded by the recited polynucleotide, the polypeptide encoded by the gene represented by the recited polynucleotide, as well as portions or fragments thereof. “Polypeptides” also includes variants of the naturally occurring proteins, where such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, usually at least about 90%, and more usually at least about 98% sequence identity with a differentially expressed polypeptide of the invention, as measured by BLAST using the parameters described above. The variant polypeptides can be naturally or non-naturally glycosylated, i.e., the polypeptide has a glycosylation pattern that differs from the glycosylation pattern found in the corresponding naturally occurring protein.

The invention also encompasses homologs of the disclosed polypeptides (or fragments thereof) where the homologs are isolated from other species, i.e. other animal or plant species, where such homologs, usually mammalian species, e.g. rodents, such as mice, rats; domestic animals, e.g., horse, cow, dog, cat; and humans. By homolog is meant a polypeptide having at least about 35%, usually at least about 40% and more usually at least about 60% amino acid sequence identity a particular differentially expressed protein as identified above, where sequence identity is determined using the BLAST algorithm, with the parameters described supra.

In general, the polypeptides of the subject invention are provided in a non-naturally occurring environment, e.g. are separated from their naturally occurring environment. In certain embodiments, the subject protein is present in a composition that is enriched for the protein as compared to a control. As such, purified polypeptide is provided, where by purified is meant that the protein is present in a composition that is substantially free of non-differentially expressed polypeptides, where by substantially free is meant that less than 90%, usually less than 60% and more usually less than 50% of the composition is made up of non-differentially expressed polypeptides.

Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. For example, substitutions between the following groups are conservative: Gly/Ala, Val/Ile/Leu, Asp/Glu, Lys/Arg, Asn/Gln, Ser/Cys, Thr, and Phe/Trp/Tyr.

Variants can be designed so as to retain biological activity of a particular region of the protein (e.g., a functional domain and/or, where the polypeptide is a member of a protein family, a region associated with a consensus sequence). In a non-limiting example, Osawa et al., Biochem. Mol. Int. (1994) 34:1003, discusses the actin binding region of a protein from several different species. The actin binding regions of the these species are considered homologous based on the fact that they have amino acids that fall within “homologous residue groups.” Homologous residues are judged according to the following groups (using single letter amino acid designations): STAG; ILVMF; HRK; DEQN; and FYW. For example, and S, a T, an A or a G can be in a position and the function (in this case actin binding) is retained.

Additional guidance on amino acid substitution is available from studies of protein evolution. Go et al, Int. J. Peptide Protein Res. (1980) 15:211, classified amino acid residue sites as interior or exterior depending on their accessibility. More frequent substitution on exterior sites was confirmed to be general in eight sets of homologous protein families regardless of their biological functions and the presence or absence of a prosthetic group. Virtually all types of amino acid residues had higher mutabilities on the exterior than in the interior. No correlation between mutability and polarity was observed of amino acid residues in the interior and exterior, respectively. Amino acid residues were classified into one of three groups depending on their polarity: polar (Arg, Lys, His, Gln, Asn, Asp, and Glu); weak polar (Ala, Pro, Gly, Thr, and Ser), and nonpolar (Cys, Val, Met, Ile, Leu, Phe, Tyr, and Trp). Amino acid replacements during protein evolution were very conservative: 88% and 76% of them in the interior or exterior, respectively, were within the same group of the three. Inter-group replacements are such that weak polar residues are replaced more often by nonpolar residues in the interior and more often by polar residues on the exterior.

Additional guidance for production of polypeptide variants is provided in Querol et al., Prot. Eng. (1996) 9:265, which provides general rules for amino acid substitutions to enhance protein thermostability. New glycosylation sites can be introduced as discussed in Olsen and Thomsen, J. Gen. Microbiol. (1991) 137:579. An additional disulfide bridge can be introduced, as discussed by Perry and Wetzel, Science (1984) 226:555; Pantoliano et al., Biochemistry (1987) 26:2077; Matsumura et al., Nature (1989) 342:291; Nishikawa et al., Protein Eng. (1990) 3:443; Takagi et al., J. Biol. Chem. (1990) 265:6874; Clarke et al., Biochemistry (1993) 32:4322; and Wakarchuk et al., Protein Eng. (1994) 7:1379. Metal binding sites can be introduced, according to Toma et al., Biochemistry (1991) 30:97, and Haezerbrouck et al., Protein Eng. (1993) 6:643. Substitutions with prolines in loops can be made according to Masul et al., Appl. Env. Microbiol. (1994) 60:3579; and Hardy et al., FEBS Lett. 317:89.

Cysteine-depleted muteins are considered variants within the scope of the invention. These variants can be constructed according to methods disclosed in U.S. Pat. No. 4,959,314, which discloses substitution of cysteines with other amino acids, and methods for assaying biological activity and effect of the substitution. Such methods are suitable for proteins according to this invention that have cysteine residues suitable for such substitutions, for example to eliminate disulfide bond formation.

Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a polynucleotide having a sequence of any SEQ ID NOS:1-844, or a homolog thereof.

The protein variants described herein are encoded by polynucleotides that are within the scope of the invention. The genetic code can be used to select the appropriate codons to construct the corresponding variants.

VI. Computer-Related Embodiments

In general, a library of polynucleotides is a collection of sequence information, which information is provided in either biochemical form (e.g., as a collection of polynucleotide molecules), or in electronic form (e.g., as a collection of polynucleotide sequences stored in a computer-readable form, as in a computer system and/or as part of a computer program). The sequence information of the polynucleotides can be used in a variety of ways, e.g., as a resource for gene discovery, as a representation of sequences expressed in a selected cell type (e.g., cell type markers), and/or as markers of a given disease or disease state. In general, a disease marker is a representation of a gene product that is present in all affected by disease either at an increased or decreased level relative to a normal cell (e.g., a cell of the same or similar type that is not substantially affected by disease). For example, a polynucleotide sequence in a library can be a polynucleotide that represents an mRNA, polypeptide, or other gene product encoded by the polynucleotide, that is either overexpressed or underexpressed in a breast ductal cell affected by cancer relative to a normal (i.e., substantially disease-free) breast cell.

The nucleotide sequence information of the library can be embodied in any suitable form, e.g., electronic or biochemical forms. For example, a library of sequence information embodied in electronic form includes an accessible computer data file (or, in biochemical form, a collection of nucleic acid molecules) that contains the representative nucleotide sequences of genes that are differentially expressed (e.g., overexpressed or underexpressed) as between, for example, i) a cancerous cell and a normal cell; ii) a cancerous cell and a dysplastic cell; iii) a cancerous cell and a cell affected by a disease or condition other than cancer; iv) a metastatic cancerous cell and a normal cell and/or non-metastatic cancerous cell; v) a malignant cancerous cell and a non-malignant cancerous cell (or a normal cell) and/or vi) a dysplastic cell relative to a normal cell. Other combinations and comparisons of cells affected by various diseases or stages of disease will be readily apparent to the ordinarily skilled artisan. Biochemical embodiments of the library include a collection of nucleic acids that have the sequences of the genes in the library, where the nucleic acids can correspond to the entire gene in the library or to a fragment thereof, as described in greater detail below.

The polynucleotide libraries of the subject invention include sequence information of a plurality of polynucleotide sequences, where at least one of the polynucleotides has a sequence of any of SEQ ID NOS :1-844. By plurality is meant at least 2, usually at least 3 and can include up to all of SEQ ID NOS:1-844. The length and number of polynucleotides in the library will vary with the nature of the library, e.g., if the library is an oligonucleotide array, a cDNA array, a computer database of the sequence information, etc.

Where the library is an electronic library, the nucleic acid sequence information can be present in a variety of media. “Media” refers to a manufacture, other than an isolated nucleic acid molecule, that contains the sequence information of the present invention. Such a manufacture provides the genome sequence or a subset thereof in a form that can be examined by means not directly applicable to the sequence as it exists in a nucleic acid. For example, the nucleotide sequence of the present invention, e.g. the nucleic acid sequences of any of the polynucleotides of SEQ ID NOS:1-844, can be recorded on computer readable media, e.g. any medium that can be read and accessed directly by a computer. Such media include, but are not limited to: magnetic storage media, such as a floppy disc, a hard disc storage medium, and a magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present sequence information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc. In addition to the sequence information, electronic versions of the libraries of the invention can be provided in conjunction or connection with other computer-readable information and/or other types of computer-readable files (e.g., searchable files, executable files, etc, including, but not limited to, for example, search program software, etc.).

By providing the nucleotide sequence in computer readable form, the information can be accessed for a variety of purposes. Computer software to access sequence information is publicly available. For example, the BLAST (Altschul et al., supra.) and BLAZE (Brutlag et al. Comp. Chem. (1993) 17:203) search algorithms on a Sybase system can be used identify open reading frames (ORFs) within the genome that contain homology to ORFs from other organisms.

As used herein, “a computer-based system” refers to the hardware means, software means, and data storage means used to analyze the nucleotide sequence information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means can comprise any manufacture comprising a recording of the present sequence information as described above, or a memory access means that can access such a manufacture.

“Search means” refers to one or more programs implemented on the computer-based system, to compare a target sequence or target structural motif with the stored sequence information. Search means are used to identify fragments or regions of the genome that match a particular target sequence or target motif. A variety of known algorithms are publicly known and commercially available, e.g. MacPattern (EMBL), BLASTN and BLASTX (NCBI). A “target sequence” can be any DNA or amino acid sequence of six or more nucleotides or two or more amino acids, preferably from about 10 to 100 amino acids or from about 30 to 300 nucleotide residues.

A “target structural motif,” or “target motif,” refers to any rationally selected sequence or combination of sequences in which the sequence(s) are chosen based on a three-dimensional configuration that is formed upon the folding of the target motif, or on consensus sequences of regulatory or active sites. There are a variety of target motifs known in the art. Protein target motifs include, but arc not limited to, enzyme active sites and signal sequences. Nucleic acid target motifs include, but are not limited to, hairpin structures, promoter sequences and other expression elements such as binding sites for transcription factors.

A variety of structural formats for the input and output means can be used to input and output the information in the computer-based systems of the present invention. One format for an output means ranks fragments of the genome possessing varying degrees of homology to a target sequence or target motif. Such presentation provides a skilled artisan with a ranking of sequences and identifies the degree of sequence similarity contained in the identified fragment.

A variety of comparing means can be used to compare a target sequence or target motif with the data storage means to identify sequence fragments of the genome. A skilled artisan can readily recognize that any one of the publicly available homology search programs can be used as the search means for the computer based systems of the present invention.

As discussed above, the “library” of the invention also encompasses biochemical libraries of the polynucleotides of SEQ ID NOS:1-844, e.g., collections of nucleic acids representing the provided polynucleotides. The biochemical libraries can take a variety of forms, e.g., a solution of cDNAs, a pattern of probe nucleic acids stably associated with a surface of a solid support (i.e., an array) and the like. Of particular interest are nucleic acid arrays in which one or more of SEQ ID NOS:1-844 is represented on the array. By array is meant a an article of manufacture that has at least a substrate with at least two distinct nucleic acid targets on one of its surfaces, where the number of distinct nucleic acids can be considerably higher, typically being at least 10 nt, usually at least 20 nt and often at least 25 nt. A variety of different array formats have been developed and are known to those of skill in the art, including those described in U.S. Pat. Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,895; 5,624,711; 5,639,603; 5,658,734; WO 93/17126; WO 95/11995; WO 95/35505; EP 742287; and EP 799897. The arrays of the subject invention find use in a variety of applications, including gene expression analysis, drug screening, mutation analysis and the like, as disclosed in the above-listed exemplary patent documents.

In addition to the above nucleic acid libraries, analogous libraries of polypeptides are also provided, where the where the polypeptides of the library will represent at least a portion of the polypeptides encoded by SEQ ID NOS:1-844.

VII. Utilities

A. Use of Polynucleotide Probes in Mapping, and in Tissue Profiling

Polynucleotide probes, generally comprising at least 12 contiguous nucleotides of a polynucleotide as shown in the Sequence Listing, are used for a variety of purposes, such as chromosome mapping of the polynucleotide and detection of transcription levels. Additional disclosure about preferred regions of the disclosed polynucleotide sequences is found in the Examples. A probe that hybridizes specifically to a polynucleotide disclosed herein should provide a detection signal at least 5-, 10-, or 20-fold higher than the background hybridization provided with other unrelated sequences.

Probes in Detection of Expression Levels.

Nucleotide probes are used to detect expression of a gene corresponding to the provided polynucleotide. The references describe an example of a sandwich nucleotide hybridization assay. For example, in Northern blots, mRNA is separated electrophoretically and contacted with a probe. A probe is detected as hybridizing to an mRNA species of a particular size. The amount of hybridization is quantitated to determine relative amounts of expression, for example under a particular condition. Probes are also used to detect products of amplification by polymerase chain reaction. The products of the reaction are hybridized to the probe and hybrids are detected. Probes are used for in situ hybridization to cells to detect expression. Probes can also be used in vivo for diagnostic detection of hybridizing sequences. Probes are typically labeled with a radioactive isotope. Other types of detectable labels can be used such as chromophores, fluors, and enzymes. Other examples of nucleotide hybridization assays are described in WO92/02526 and U.S. Pat. No. 5,124,246.

Alternatively, the Polymerase Chain Reaction (PCR) is another means for detecting small amounts of target nucleic acids (see, e.g., Mullis et al., Meth. Enzymol. (1987) 155:335; U.S. Pat. No. 4,683,195; and U.S. Pat. No. 4,683,202). Two primer polynucleotides nucleotides hybridize with the target nucleic acids and are used to prime the reaction. The primers can be composed of sequence within or 3′ and 5′ to the polynucleotides of the Sequence Listing. Alternatively, if the primers are 3′ and 5′ to these polynucleotides, they need not hybridize to them or the complements. A thermostable polymerase creates copies of target nucleic acids from the primers using the original target nucleic acids as a template. After a large amount of target nucleic acids is generated by the polymerase, it is detected by methods such as Southern blots. When using the Southern blot method, the labeled probe will hybridize to a polynucleotide of the Sequence Listing or complement.

Furthermore, mRNA or cDNA can be detected by traditional blotting techniques described in Sambrook et al., “Molecular Cloning: A Laboratory Manual” (New York, Cold Spring Harbor Laboratory, 1989). mRNA or cDNA generated from mRNA using a polymerase enzyme can be purified and separated using gel electrophoresis. The nucleic acids on the gel are then blotted onto a solid support, such as nitrocellulose. The solid support is exposed to a labeled probe and then washed to remove any unhybridized probe. Next, the duplexes containing the labeled probe are detected. Typically, the probe is labeled with radioactivity.

Mapping.

Polynucleotides of the present invention are used to identify a chromosome on which the corresponding gene resides. Such mapping can be useful in identifying the function of the polynucleotide-related gene by its proximity to other genes with known function. Function can also be assigned to the polynucleotide-related gene when particular syndromes or diseases map to the same chromosome. For example, use of polynucleotide probes in identification and quantification of nucleic acid sequence aberrations is described in U.S. Pat. No. 5,783,387.

For example, fluorescence in situ hybridization (FISH) on normal metaphase spreads facilitates comparative genomic hybridization to allow total genome assessment of changes in relative copy number of DNA sequences. See Schwartz and Samad, Curr. Opin. Biotechnol. (1994) 8:70; Kallioniemi et al., Sem. Cancer Biol. (1993) 4:41; Valdes et al., Methods in Molecular Biology (1997) 68: 1, Boultwood, ed., Human Press, Totowa, N.J. Preparations of human metaphase chromosomes are prepared using standard cytogenetic techniques from human primary tissues or cell lines. Nucleotide probes comprising at least 12 contiguous nucleotides selected from the nucleotide sequence shown in the Sequence Listing are used to identify the corresponding chromosome. The nucleotide probes are labeled, for example, with a radioactive, fluorescent, biotinylated, or chemiluminescent label, and detected by well known methods appropriate for the particular label selected. Protocols for hybridizing nucleotide probes to preparations of metaphase chromosomes are also well known in the art. A nucleotide probe will hybridize specifically to nucleotide sequences in the chromosome preparations that are complementary to the nucleotide sequence of the probe.

Polynucleotides are mapped to particular chromosomes using, for example, radiation hybrids or chromosome-specific hybrid panels. See Leach et al., Advances in Genetics, (1995) 33:63-99; Walter et al., Nature Genetics (1994) 7:22; Walter and Goodfellow, Trends in Genetics (1992) 9:352. Panels for radiation hybrid mapping are available from Research Genetics, Inc., Huntsville, Ala., USA. Databases for markers using various panels are available via the world wide web at http:/F/shgc-www.stanford.edu; and http://www-genome.wi.mit.edu/cgi-bin/contig/rhmapper.pl. The statistical program RHMAP can be used to construct a map based on the data from radiation hybridization with a measure of the relative likelihood of one order versus another. RHMAP is available via the world wide web at http://www.sph.umich.edu/group/statgen/software.

In addition, commercial programs are available for identifying regions of chromosomes commonly associated with disease, such as cancer. Polynucleotides based on the polynucleotides of the invention can be used to probe these regions. For example, if through profile searching a provided polynucleotide is identified as corresponding to a gene encoding a kinase, its ability to bind to a cancer-related chromosomal region will suggest its role as a kinase in one or more stages of tumor cell development/growth. Although some experimentation would be required to elucidate the role, the polynucleotide constitutes a new material for isolating a specific protein that has potential for developing a cancer diagnostic or therapeutic.

Tissue Typing or Profiling.

Expression of specific mRNA corresponding to the provided polynucleotides can vary in different cell types and can be tissue-specific. This variation of mRNA levels in different cell types can be exploited with nucleic acid probe assays to determine tissue types. For example, PCR, branched DNA probe assays, or blotting techniques utilizing nucleic acid probes substantially identical or complementary to polynucleotides listed in the Sequence Listing can determine the presence or absence of the corresponding cDNA or mRNA.

For example, a metastatic lesion is identified by its developmental organ or tissue source by identifying the expression of a particular marker of that organ or tissue. If a polynucleotide is expressed only in a specific tissue type, and a metastatic lesion is found to express that polynucleotide, then the developmental source of the lesion has been identified. Expression of a particular polylucleotide is assayed by detection of either the corresponding mRNA or the protein product. Immunological methods, such as antibody staining, are used to detect a particular protein product. Hybridization methods can be used to detect particular mRNA species, including but not limited to in situ hybridization and Northern blotting.

Use of Polymorphisms.

A polynucleotide of the invention will be useful in forensics, genetic analysis, mapping, and diagnostic applications if the corresponding region of a gene is polymorphic in the human population. Particular polymorphic forms of the provided polynucleotides can be used to either identify a sample as deriving from a suspect or rule out the possibility that the sample derives from the suspect. Any means for detecting a polymorphism in a gene are used, including but not limited to electrophoresis of protein polymorphic variants, differential sensitivity to restriction enzyme cleavage, and hybridization to allele-specific probes.

B. Antibody Production

Expression products of a polynucleotide of the invention, the corresponding mRNA or cDNA, or the corresponding complete gene are prepared and used for raising antibodies for experimental, diagnostic, and therapeutic purposes. For polynucleotides to which a corresponding gene has not been assigned, this provides an additional method of identifying the corresponding gene. The polynucleotide or related cDNA is expressed as described above, and antibodies are prepared. These antibodies are specific to an epitope on the polypeptide encoded by the polynucleotide, and can precipitate or bind to the corresponding native protein in a cell or tissue preparation or in a cell-free extract of an in vitro expression system.

Immunogens for raising antibodies are prepared by mixing the polypeptides encoded by the polynucleotides of the present invention with adjuvants. Alternatively, polypeptides are made as fusion proteins to larger immunogenic proteins. Polypeptides are also covalently linked to other larger immunogenic proteins, such as keyhole limpet hemocyanin. Immunogens are typically administered intradermally, subcutaneously, or intramuscularly. Immunogens are administered to experimental animals such as rabbits, sheep, and mice, to generate antibodies. Optionally, the animal spleen cells are isolated and fused with myeloma cells to form hybridomas which secrete monoclonal antibodies. Such methods are well known in the art. According to another method known in the art, the selected polynucleotide is administered directly, such as by intramuscular injection, and expressed in vivo. The expressed protein generates a variety of protein-specific immune responses, including production of antibodies, comparable to administration of the protein.

Preparations of polyclonal and monoclonal antibodies specific for polypeptides encoded by a selected polynucleotide are made using standard methods known in the art. The antibodies specifically bind to epitopes present in the polypeptides encoded by polynucleotides disclosed in the Sequence Listing. Typically, at least 6, 8, 10, or 12 contiguous amino acids are required to form an epitope. However, epitopes which involve non-contiguous amino acids may require more, for example at least 15, 25, or 50 amino acids. A short sequence of a polynucleotide may then be unsuitable for use as an epitope to raise antibodies for identifying the corresponding novel protein, because of the potential for cross-reactivity with a known protein. However, the antibodies can be useful for other purposes, particularly if they identify common structural features of a known protein and a novel polypeptide encoded by a polynucleotide of the invention.

Antibodies that specifically bind to human polypeptides encoded by the provided polypeptides should provide a detection signal at least 5-, 10-, or 20-fold higher than a detection signal provided with other proteins when used in Western blots or other immunochemical assays. Preferably, antibodies that specifically polypeptides of the invention do not bind to other proteins in immunochemical assays at detectable levels and can immunoprecipitate the specific polypeptide from solution.

To test for the presence of serum antibodies to the polypeptide of the invention in a human population, human antibodies are purified by methods well known in the art. Preferably, the antibodies are affinity purified by passing antiserum over a column to which the corresponding selected polypeptide or fiusion protein is bound. The bound antibodies can then be eluted from the column, for example using a buffer with a high salt concentration.

In addition to the antibodies discussed above, genetically engineered antibody derivatives are made, such as single chain antibodies, according to methods well known in the art.

C. Use of Polynucleotides to Construct Arrays for Diagnostics

Polynucleotide arrays provide a high throughput technique that can assay a large number of polynucleotide sequences in a sample. This technology can be used as a diagnostic and as a tool to test for differential expression to determine function of an encoded protein. Arrays can be created by spotting polynucleotide probes onto a substrate (e.g., glass, nitrocelllose, etc.) in a two-dimensional matrix or array having bound probes. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. No. 5,593,839; U.S. Pat. No. 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734.

As discussed in some detail above, arrays can be used to examine differential expression of genes and can be used to determine gene function. For example, arrays of the instant polynucleotide sequences can be used to determine if any of the provided polynucleotides are differentially expressed between a test cell and control cell (e.g., cancer cells and normal cells). For example, high expression of a particular message in a cancer cell, which is not observed in a corresponding normal cell, can indicate a cancer specific protein. Exemplary uses of arrays are further described in, for example, Pappalarado et al., Sem. Radiation Oncol. (1998) 8:217; and Ramsay Nature Biotechnol. (1998) 16:40.

D. Differential Exipression

The polynucleotides of the invention can also be used to detect differences in expression levels between two cells, e.g, as a method to identify abnormal or diseased tissue in a human. For polynucleotides corresponding to profiles of protein families as described above, the choice of tissue can be selected according to the putative biological function. In general, the expression of a gene corresponding to a specific polynucleotide is compared between a first tissue that is suspected of being diseased and a second, normal tissue of the human. The tissue suspected of being abnormal or diseased can be derived from a different tissue type of the human, but preferably it is derived from the same tissue type; for example an intestinal polyp or other abnormal growth should be compared with normal intestinal tissue. The normal tissue can be the same tissue as that of the test sample, or any normal tissue of the patient, especially those that express the polynucleotide-related gene of interest (e.g, brain, thymus, testis, heart, prostate, placenta, spleen, small intestine, skeletal muscle, pancreas, and the mucosal lining of the colon). A difference between the polynucleotide-related gene, mRNA, or protein in the two tissues which are compared, for example in molecular weight, amino acid or nucleotide sequence, or relative abundance, indicates a change in the gene, or a gene which regulates it, in the tissue of the human that was suspected of being diseased. Examples of detection of differential expression and its use in diagnosis of cancer are described in U.S. Pat. Nos. 5,688,641 and 5,677,125.

The polynucleotide-related genes in the two tissues are compared by any means known in the art. For example, the two genes can be sequenced, and the sequence of the gene in the tissue suspected of being diseased compared with the gene sequence in the normal tissue. The genes corresponding to a provided polynucleotide, or portions thereof, in the two tissues are amplified, for example using nucleotide primers based on the nucleotide sequence shown in the Sequence Listing, using the polymerase chain reaction. The amplified genes or portions of genes are hybridized to detectably labeled nucleotide probes selected from a nucleotide sequence shown in the Sequence Listing. A difference in the nucleotide sequence of the isolated gene in the tissue suspected of being diseased compared with the normal nucleotide sequence suggests a role of the gene product encoded by the subject polynucleotide in the disease, and provides guidance for preparing a therapeutic agent.

Alternatively, mRNA corresponding to a provided polynucleotide in the two tissues is compared. PolyA⁺RNA is isolated from the two tissues as is known in the art. For example, one of skill in the art can readily determine differences in the size or amount of mRNA transcripts between the two tissues using Northern blots and detectably labeled nucleotide probes selected from the nucleotide sequence shown in the Sequence Listing. Increased or decreased expression of a given mRNA in a tissue sample suspected of being diseased, compared with the expression of the same mRNA in a normal tissue, suggests that the expressed protein has a role in the disease, and also provides a lead for preparing a therapeutic agent.

The comparison can also be accomplished by analyzing polypeptides between the matched samples. The sizes of the proteins in the two tissues are compared, for example, using antibodies of the present invention to detect polypeptides in Western blots of protein extracts from the two tissues. Other changes, such as expression levels and subcellular localization, can also be detected immunologically, using antibodies to the corresponding protein. A higher or lower level of expression of a given polypeptide in a tissue suspected of being diseased, compared with the same protein expression level in a normal tissue, is indicative that the expressed protein has a role in the disease, and provides guidance for preparing a therapeutic agent.

Similarly, comparison of polynucleotide sequences or of gene expression products, e.g., mRNA and protein, between a human tissue that is suspected of being diseased and a normal tissue of a human, are used to follow disease progression or remission in the human. Such comparisons are made as described above. For example, increased or decreased expression of a gene corresponding to an inventive polynucleotide in the tissue suspected of being neoplastic can indicate the presence of neoplastic cells in the tissue. The degree of increased expression of a given gene in the neoplastic tissue relative to expression of the same gene in normal tissue, or differences in the amount of increased expression of a given gene in the neoplastic tissue over time, is used to assess the progression of the neoplasia in that tissue or to monitor the response of the neoplastic tissue to a therapeutic protocol over time.

The expression pattern of any two cell types can be compared, such as low and high metastatic tumor cell lines, malignant or non-malignant cells, or cells from tissue which have and have not been exposed to a therapeutic agent. A genetic predisposition to disease in a human is detected by comparing expression levels of an mRNA or protein corresponding to a polynucleotide of the invention in a fetal tissue with levels associated in normal fetal tissue. Fetal tissues that are used for this purpose include, but are not limited to, amniotic fluid, chorionic villi, blood, and the blastomere of an in vitro-fertilized embryo. The comparable normal polynucleotide-related gene is obtained from any tissue. The mRNA or protein is obtained from a normal tissue of a human in which the polynucleotide-related gene is expressed. Differences such as alterations in the nucleotide sequence or size of the same product of the fetal polynucleotide-related gene or mRNA, or alterations in the molecular weight, amino acid sequence, or relative abundance of fetal protein, can indicate a germline mutation in the polynucleotide-related gene of the fetus, which indicates a genetic predisposition to disease. Particular diagnostic and prognostic uses of the disclosed polynucleotides are described in more detail below.

E. Diagnostic, Prognostic, and Other Uses Based on Differential Expression

In general, diagnostic methods of the invention for involve detection of a level or amount of a gene product, particularly a differentially expressed gene product, in a test sample obtained from a patient suspected of having or being susceptible to a disease (e.g., breast cancer, lung cancer, colon cancer and/or metastatic forms thereof), and comparing the detected levels to those levels found in normal cells (e.g., cells substantially unaffected by cancer) and/or other control cells (e.g., to differentiate a cancerous cell from a cell affected by dysplasia). Furthermore, the severity of the disease can be assessed by comparing the detected levels of a differentially expressed gene product with those levels detected in samples representing the levels of differentially gene product associated with varying degrees of severity of disease.

The term “differentially expressed gene” is intended to encompass a polynucleotide that can, for example, include an open reading frame encoding a gene product (e.g., a polypeptide), and/or introns of such genes and adjacent 5′ and 3′ non-coding nucleotide sequences involved in the regulation of expression, up to about 20 kb beyond the coding region, but possibly further in either direction. The gene can be introduced into an appropriate vector for extrachromosomal maintenance or for integration into a host genome. In general, a difference in expression level associated with a decrease in expression level of at least about 25%, usually at least about 50% to 75%, more usually at least about 90% or more is indicative of a differentially expressed gene of interest, i.e., a gene that is underexpressed or down-regulated in the test sample relative to a control sample. Furthermore, a difference in expression level associated with an increase in expression of at least about 25%, usually at least about 50% to 75%, more usually at least about 90% and can be at least about 1½-fold, usually at least about 2-fold to about 10-fold, and can be about 100-fold to about 1,000-fold increase relative to a control sample is indicative of a differentially expressed gene of interest, i.e., an overexpressed or up-regulated gene.

“Differentially expressed polynucleotide” as used herein means a nucleic acid molecule (RNA or DNA) having a sequence that represents a differentially expressed gene, e.g., the differentially expressed polynucleotide comprises a sequence (e.g, an open reading frame encoding a gene product) that uniquely identifies a differentially expressed gene so that detection of the differentially expressed polynucleotide in a sample is correlated with the presence of a differentially expressed gene in a sample. “Differentially expressed polynucleotides” is also meant to encompass fragments of the disclosed polynucleotides, e.g., fragments retaining biological activity, as well as nucleic acids homologous, substantially similar, or substantially identical (e.g., having about 90% sequence identity) to the disclosed polynucleotides.

Methods of the subject invention useful in diagnosis or prognosis typically involve comparison of the abundance of a selected differentially expressed gene product in a sample of interest with that of a control to determine any relative differences in the expression of the gene product, where the difference can be measured qualitatively and/or quantitatively. Quantitation can be accomplished, for example, by comparing the level of expression product detected in the sample with the amounts of product present in a standard curve. A comparison can be made visually; by using a technique such as densitometry, with or without computerized assistance; by preparing a representative library of cDNA clones of mRNA isolated from a test sample, sequencing the clones in the library to determine that number of cDNA clones corresponding to the same gene product, and analyzing the number of clones corresponding to that same gene product relative to the number of clones of the same gene product in a control sample; or by using an array to detect relative levels of hybridization to a selected sequence or set of sequences, and comparing the hybridization pattern to that of a control. The differences in expression are then correlated with the presence or absence of an abnormal expression pattern. A variety of different methods for determining the nucleic acid abundance in a sample are known to those of skill in the art, where particular methods of interest include those described in: Pietu et al. Genome Res. (1996) 6:492; Zhao et al., Gene (1995) 156:207; Soares, Curr. Opin. Biotechnol. (1 977) 8: 542; Raval, J. Pharmacol Toxicol Methods (1994) 32:125; Chalifour et al., Anal. Biochem (1994) 216:299; Stolz et al., Mol. Biotechnol. (1996) 6:225; Hong et al., Biosci. Reports (1982) 2:907; and McGraw, Anal. Biochem. (1984) 143:298. Also of interest are the methods disclosed in WO 97/27317, the disclosure of which is herein incorporated by reference.

In general, diagnostic assays of the invention involve detection of a gene product of a the polynucleotide sequence (e.g., mRNA or polypeptide) that corresponds to a sequence of SEQ ID NOS:1-844. The patient from whom the sample is obtained can be apparently healthy, susceptible to disease (e.g., as determined by family history or exposure to certain environmental factors), or can already be identified as having a condition in which altered expression of a gene product of the invention is implicated.

In the assays of the invention, the diagnosis can be determined based on detected gene product expression levels of a gene product encoded by at least one, preferably at least two or more, at least 3 or more, or at least 4 or more of the polynucleotides having a sequence set forth in SEQ ID NOS:1-844, and can involve detection of expression of genes corresponding to all of SEQ ID NOS:1-844 and/or additional sequences that can serve as additional diagnostic markers and/or reference sequences. Where the diagnostic method is designed to detect the presence or susceptibility of a patient to cancer, the assay preferably involves detection of a gene product encoded by a gene corresponding to a polynucleotide that is differentially expressed in cancer. For example, a higher level of expression of a polynucleotide corresponding to SEQ ID NO:52 relative to a level associated with a noimal sample can indicate the presence of cancer in the patient from whom the sample is derived. In another example, detection of a lower level of a polynucleotide corresponding to SEQ ID NO:39 relative to a normal level is indicative of the presence of cancer in the patient. Further examples of such differentially expressed polynucleotides are described in the Examples below. Given the provided polynucleotides and information regarding their relative expression levels provided herein, assays using such polynucleotides and detection of their expression levels in diagnosis and prognosis will be readily apparent to the ordinarily skilled artisan.

Any of a variety of detectable labels can be used in connection with the various embodiments of the diagnostic methods of the invention. Suitable detectable labels include fluorochromes,(e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g. ³²P, ³⁵S, ³H, etc.), and the like. The detectable label can involve a two stage systems (e.g., biotin-avidin, hapten-anti-hapten antibody, etc.)

Reagents specific for the polynucleotides and polypeptides of the invention, such as antibodies and nucleotide probes, can be supplied in a kit for detecting the presence of an expression product in a biological sample. The kit can also contain buffers or labeling components, as well as instructions for using the reagents to detect and quantify expression products in the biological sample. Exemplary embodiments of the diagnostic methods of the invention are described below in more detail.

Polypeptide Detection in Diagnosis.

In one embodiment, the test sample is assayed for the level of a differentially expressed polypeptide. Diagnosis can be accomplished using any of a number of methods to determine the absence or presence or altered amounts of the differentially expressed polypeptide in the test sample. For example, detection can utilize staining of cells or histological sections with labeled antibodies, performed in accordance with conventional methods. Cells can be permneabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be used in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

In general, the detected level of differentially expressed polypeptide in the test sample is compared to a level of the differentially expressed gene product in a reference or control sample, e.g., in a normal cell (negative control) or in a cell having a known disease state (positive control). For example, a higher level of expression of a polypeptide encoded by SEQ ID NO:52 relative to a level associated with a normal sample can indicate the presence of cancer in the patient from whom the sample is derived. In another example, detection of a lower level of the polypeptide encoded by SEQ ID NO:39 relative to a normal level is indicative of the presence of cancer in the patient.

mRNA Detection.

The diagnostic methods of the invention can also or alternatively involve detection of mRNA encoded by a gene corresponding to a differentially expressed polynucleotides of the invention. Any suitable qualitative or quantitative methods known in the art for detecting specific mRNAs can be used. mRNA can be detected by, for example, in situ hybridization in tissue sections, by reverse transcriptase-PCR, or in Northern blots containing poly A+ mRNA. One of skill in the art can readily use these methods to determine differences in the size or amount of mRNA transcripts between two samples. For example, the level of mRNA of the invention in a tissue sample suspected of being cancerous or dysplastic is compared with the expression of the mRNA in a reference sample, e.g., a positive or negative control sample (e.g., normal tissue, cancerous tissue, etc.). In a specific non-limiting example, a higher level of mRNA corresponding to SEQ ID NO:52 relative to a level associated with a normal sample can indicate the presence of cancer in the patient from whom the sample is derived. In another example, detection of a lower level of mRNA corresponding to SEQ ID NO:39 relative to a normal level is indicative of the presence of cancer in the patient.

Any suitable method for detecting and comparing mRNA expression levels in a sample can be used in connection with the diagnostic methods of the invention (see, e.g., U.S. Pat. No. 5,804,382). For example, mRNA expression levels in a sample can be determined by generation of a library of expressed sequence tags (ESTs) from the sample, where the EST library is representative of sequences present in the sample (Adams, et al., (1991) Science 252:1651). Enumeration of the relative representation of ESTs within the library can be used to approximate the relative representation of the gene transcript within the starting sample. The results of EST analysis of a test sample can then be compared to EST analysis of a reference sample to determine the relative expression levels of a selected polynucleotide, particularly a polynucleotide corresponding to one or more of the differentially expressed genes described herein.

Alternatively, gene expression in a test sample can be performed using serial analysis of gene expression (SAGE) methodology (Velculescu et al., Science (1995) 270:484). In short, SAGE involves the isolation of short unique sequence tags from a specific location within each transcript (e.g, a sequence of any one of SEQ ID NOS:1-6). The sequence tags are concatenated, cloned, and sequenced. The frequency of particular transcripts within the starting sample is reflected by the number of times the associated sequence tag is encountered with the sequence population.

Gene expression in a test sample can also be analyzed using differential display (DD) methodology. In DD, fragments defined by specific sequence delimiters (e.g., restriction enzyme sites) are used as unique identifiers of genes, coupled with information about fragment length or fragment location within the expressed gene. The relative representation of an expressed gene with a sample can then be estimated based on the relative representation of the fragment associated with that gene within the pool of all possible fragments. Methods and compositions for carrying out DD are well known in the art, see, e.g., U.S. Pat. No. 5,776,683; and U.S. Pat. No. 5,807,680.

Alternatively, gene expression in a sample using hybridization analysis, which is based on the specificity of nucleotide interactions. Oligonucleotides or cDNA can be used to selectively identify or capture DNA or RNA of specific sequence composition, and the amount of RNA or cDNA hybridized to a known capture sequence determined qualitatively or quantitatively, to provide information about the relative representation of a particular message within the pool of cellular messages in a sample. Hybridization analysis can be designed to allow for concurrent screening of the relative expression of hundreds to thousands of genes by using, for example, array-based technologies having high density formats, including filters, microscope slides, or microchips, or solution-based technologies that use spectroscopic analysis (e.g., mass spectrometry). One exemplary use of arrays in the diagnostic methods of the invention is described below in more detail.

Use of a Single Gene in Diagnostic Applications.

The diagnostic methods of the invention can focus on the expression of a single differentially expressed gene. For example, the diagnostic method can involve detecting a differentially expressed gene, or a polymorphism of such a gene (e.g., a polymorphism in an coding region or control region), that is associated with disease. Disease-associated polymorphisms can include deletion or truncation of the gene, mutations that alter expression level and/or affect activity of the encoded protein, etc.

Changes in the promoter or enhancer sequence that affect expression levels of an differentially gene can be compared to expression levels of the normal allele by various methods known in the art. Methods for determining promoter or enhancer strength include quantitation of the expressed natural protein; insertion of the variant control element into a vector with a reporter gene such as β-galactosidase, luciferase, chloramphenicol acetyltransferase, etc. that provides for convenient quantitation; and the like.

A number of methods are available for analyzing nucleic acids for the presence of a specific sequence, e.g. a disease associated polymorphism. Where large amounts of DNA are available, genomic DNA is used directly. Alternatively, the region of interest is cloned into a suitable vector and grown in sufficient quantity for analysis. Cells that express a differentially expressed gene can be used as a source of mRNA, which can be assayed directly or reverse transcribed into cDNA for analysis. The nucleic acid can be amplified by conventional techniques, such as the polymerase chain reaction (PCR), to provide sufficient amounts for analysis, and a detectable label can be included in the amplification reaction (e.g., using a detectably labeled primer or detectably labeled oligonucleotides) to facilitate detection. The use of the polymerase chain reaction is described in Saiki, et al., Science (1985) 239:487, and a review of techniques can be found in Sambrook, et al., Molecular Cloning: A Laboratory Manual, (1989) pp. 14.2. Alternatively, various methods are known in the art that utilize oligonucleotide ligation as a means of detecting polymorphisms, for examples see Riley et al., Nucl. Acids Res. (1990) 18:2887; and Delahunty et al., Am. J. Hum. Genet. (1996) 58:1239.

The sample nucleic acid, e.g. amplified or cloned fragment, is analyzed by one of a number of methods known in the art. The nucleic acid can be sequenced by dideoxy or other methods, and the sequence of bases compared to a selected sequence, e.g., to a wild-type sequence. Hybridization with the polymorphic or variant sequence can also be used to determine its presence in a sample (e.g., by Southern blot, dot blot, etc). The hybridization pattern of a polymorphic or variant sequence and a control sequence to an array of oligonucleotide probes immobilized on a solid support, as described in U.S. Pat. No. 5,445,934, or in WO 95/35505, can also be used as a means of identifying polymorphic or variant sequences associated with disease. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Alternatively, where a polymorphism creates or destroys a recognition site for a restriction endonuclease, the sample is digested with that endonuclease, and the products size fractionated to determine whether the fragment was digested. Fractionation is performed by gel or capillary electrophoresis, particularly acrylamide or agarose gels.

Screening for mutations in an differentially expressed gene can be based on the functional or antigenic characteristics of the protein. Protein truncation assays are useful in detecting deletions that can affect the biological activity of the protein. Various immunoassays designed to detect polymorphisms in proteins can be used in screening. Where many diverse genetic mutations lead to a particular disease phenotype, functional protein assays have proven to be effective screening tools. The activity of the encoded protein can be determined by comparison with the wild-type protein.

Pattern Matching in Diagnosis Using Arrays.

In another embodiment, the diagnostic and/or prognostic methods of the invention involve detection of expression of a selected set of genes in a test sample to produce a test expression pattern (TEP). The TEP is compared to a reference expression pattern (REP), which is generated by detection of expression of the selected set of genes in a reference sample (e.g., a positive or negative control sample). The selected set of genes includes at least one of the genes of the invention, which genes correspond to the polynucleotide sequences of SEQ ID NOS:1-844. Of particular interest is a selected set of genes that includes gene differentially expressed in the disease for which the test sample is to be screened.

“Reference sequences” or “reference polynucleotides” as used herein in the context of differential gene expression analysis and diagnosis/prognosis refers to a selected set of polynucleotides, which selected set includes at least one or more of the differentially expressed polynucleotides described herein. A plurality of reference sequences, preferably comprising positive and negative control sequences, can be included as reference sequences. Additional suitable reference sequences are found in Genbank, Unigene, and other nucleotide sequence databases (including, e.g., expressed sequence tag (EST), partial, and full-length sequences).

“Reference array” means an array having reference sequences for use in hybridization with a sample, where the reference sequences include all, at least one of, or any subset of the differentially expressed polynucleotides described herein. Usually such an array will include at least 3 different reference sequences, and can include any one or all of the provided differentially expressed sequences. Arrays of interest can further comprise sequences, including polymorphisms, of other genetic sequences, particularly other sequences of interest for screening for a disease or disorder (e.g., cancer, dysplasia, or other related or unrelated diseases, disorders, or conditions). The oligonucleotide sequence on the array will usually be at least about 12 nt in length, and can be of about the length of the provided sequences, or can extend into the flanking regions to generate fragments of 100 nt to 200 nt in length or more.

A “reference expression pattern” or “REP” as used herein refers to the relative levels of expression of a selected set of genes, particularly of differentially expressed genes, that is associated with a selected cell type, e.g., a normal cell, a cancerous cell, a cell exposed to an environrrental stimulus, and the like. A “test expression pattern” or “TEP” refers to relative levels of expression of a selected set of genes, particularly of differentially expressed genes, in a test sample (e.g., a cell of unknown or suspected disease state, from which mRNA is isolated).

“Diagnosis” as used herein generally includes determination of a subject's susceptibility to a disease or disorder, determination as to whether a subject is presently affected by a disease or disorder, as well as to the prognosis of a subject affected by a disease or disorder (e.g., identification of pre-metastatic or metastatic cancerous states, stages of cancer, or responsiveness of cancer to therapy). The present invention particularly encompasses diagnosis of subjects in the context of breast cancer (e.g., carcinoma in situ (e.g., ductal carcinoma in situ), estrogen receptor (ER)-positive breast cancer, ER-negative breast cancer, or other forms and/or stages of breast cancer), lung cancer (e.g., small cell carcinoma, non-small cell carcinoma, mesothelioma, and other forms and/or stages of lung cancer), and colon cancer (e.g., adenomatous polyp, colorectal carcinoma, and other forms and/or stages of colon cancer).

“Sample” or “biological sample” as used throughout here are generally meant to refer to samples of biological fluids or tissues, particularly samples obtained from tissues, especially from cells of the type associated with the disease for which the diagnostic application is designed (e.g., ductal adenocarcinoma), and the like. “Samples” is also meant to encompass derivatives and fractions of such samples (e.g., cell lysates). Where the sample is solid tissue, the cells of the tissue can be dissociated or tissue sections can be analyzed.

REPs can be generated in a variety of ways according to methods well known in the art. For example, REPs can be generated by hybridizing a control sample to an array having a selected set of polynucleotides (particularly a selected set of differentially expressed polynucleotides), acquiring the hybridization data from the array, and storing the data in a format that allows for ready comparison of the REP with a TEP. Alternatively, all expressed sequences in a control sample can be isolated and sequenced, e.g., by isolating mRNA from a control sample, converting the mRNA into cDNA, and sequencing the cDNA. The resulting sequence information roughly or precisely reflects the identity and relative number of expressed sequences in the sample. The sequence information can then be stored in a format (e.g., a computer-readable format) that allows for ready comparison of the REP with a TEP. The REP can be normalized prior to or after data storage, and/or can be processed to selectively remove sequences of expressed genes that are of less interest or that might complicate analysis (e.g., some or all of the sequences associated with housekeeping genes can be eliminated from REP data).

TEPs can be generated in a manner similar to REPs, e.g., by hybridizing a test sample to an array having a selected set of polynucleotides, particularly a selected set of differentially expressed polynucleotides, acquiring the hybridization data from the array, and storing the data in a format that allows for ready comparison of the TEP with a REP. The REP and TEP to be used in a comparison can be generated simultaneously, or the TEP can be compared to previously generated and stored REPs.

In one embodiment of the invention, comparison of a TEP with a REP involves hybridizing a test sample with a reference array, where the reference array has one or more reference sequences for use in hybridization with a sample. The reference sequences include all, at least one of, or any subset of the differentially expressed polynucleotides described herein. Hybridization data for the test sample is acquired, the data normalized, and the produced TEP compared with a REP generated using an array having the same or similar selected set of differentially expressed polynucleotides. Probes that correspond to sequences differentially expressed between the two samples will show decreased or increased hybridization efficiency for one of the samples relative to the other.

Reference arrays can be produced according to any suitable methods known in the art. For example, methods of producing large arrays of oligonucleotides are described in U.S. Pat. No. 5,134,854, and U.S. Pat. No. 5,445,934 using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of pre-synthesized oligonucleotides onto a solid substrate, for example as described in PCT published application no. WO 95/35505.

Methods for collection of data from hybridization of samples with a reference arrays are also well known in the art. For example, the polynucleotides of the reference and test samples can be generated using a detectable fluorescent label, and hybridization of the polynucleotides in the samples detected by scanning the microarrays for the presence of the detectable label. Methods and devices for detecting fluorescently marked targets on devices are known in the art. Generally, such detection devices include a microscope and light source for directing light at a substrate. A photon counter detects fluorescence from the substrate, while an x-y translation stage varies the location of the substrate. A confocal detection device that can be used in the subject methods is described in U.S. Pat. No. 5,631,734. A scanning laser microscope is described in Shalon et al., Genome Res. (1996) 6:639. A scan, using the appropriate excitation line, is performed for each fluorophore used. The digital images generated from the scan are then combined for subsequent analysis. For any particular array element, the ratio of the fluorescent signal from one sample (e.g., a test sample) is compared to the fluorescent signal from another sample (e.g., a reference sample), and the relative signal intensity determined.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e. data deviating from a predetermined statistical distribution, and calculating the relative binding affinity of the targets from the remaining data. The resulting data can be displayed as an image with the intensity in each region varying according to the binding affinity between targets and probes.

In general, the test sample is classified as having a gene expression profile corresponding to that associated with a disease or non-disease state by comparing the TEP generated from the test sample to one or more REPs generated from reference samples (e.g., from samples associated with cancer or specific stages of cancer, dysplasia, samples affected by a disease other than cancer, normal samples, etc.). The criteria for a match or a substantial match between a TEP and a REP include expression of the same or substantially the same set of reference genes, as well as expression of these reference genes at substantially the same levels (e.g., no significant difference between the samples for a signal associated with a selected reference sequence after normalization of the samples, or at least no greater than about 25% to about 40% difference in signal strength for a given reference sequence. In general, a pattern match between a TEP and a REP includes a match in expression, preferably a match in qualitative or quantitative expression level, of at least one of, all or any subset of the differentially expressed genes of the invention.

Pattern matching can be performed manually, or can be performed using a computer program. Methods for preparation of substrate matrices (e.g., arrays), design of oligonucleotides for use with such matrices, labeling of probes, hybridization conditions, scanning of hybridized matrices, and analysis of patterns generated, including comparison analysis, are described in, for example, U.S. Pat. No. 5,800,992.

F. Use of the Polynucleotides of the Invention in Cancer

Oncogenesis involves the unbridled growth, dedifferentiation and abnormal migration of cells. Cancerous cells can have the ability to compress, invade, and destroy normal tissue. Cancerous cells may also metastasize to other parts of the body via the bloodstream or the lymph system and colonize in these other areas. Different cancers are classified by the cell from which the cancerous cell is derived and from its cellular morphology and/or state of differentiation.

Somatic genetic abnormalities cause cancer initiation and progression. Cancer generally is clonally formed, i.e.gain of function of oncogenes and loss of function of tumor suppressor genes within a single cell transform the cell to be cancerous, and that single cell grows and divides to form a cancerous lesion. The genes known to be involved in cancer initiation and progression are involved in numerous cellular functions, including developmental differentiation, cell cycle regulation, cell signaling, immunological response, DNA replication, and DNA repair.

The identification and characterization of genetic or biochemical markers in blood or tissues that will detect the earliest changes along the carcinogenesis pathway and monitor the efficacy of various therapies and preventive interventions is a major goal of cancer research. Scientists have identified genetic changes in stool specimens that indicate the stages of colon cancer, and other biomarkers such as gene mutations, hormone receptors, proteins that inhibit metastasis, and enzymes that metabolize drugs are all being used to determine the severity and predict the course of breast, prostate, lung, and other cancers.

Recent advances in the pathogenesis of certain cancers has been helpful in determining patient treatment. The level of expression of certain polynucleotides can be indicative of a poorer prognosis, and therefore warrant more aggressive chemo- or radio-therapy for a patient. The correlation of novel surrogate tumor specific features with response to treatment and outcome in patients has defined certain prognostic indicators that allow the design of tailored therapy based on the molecular profile of the tumor. These therapies include antibody targeting and gene therapy. Moreover, a promising level of one or more marker polynucleotides can provide impetus for not aggressively treating a particular patient, thus sparing the patient the deleterious side effects of aggressive therapy. Determining expression of certain polynucleotides and comparison of a patients profile with known expression in normal tissue and variants of the disease allows a determination of the best possible treatment for a patient, both in terms of specificity of treatment and in terms of comfort level of the patient.

Surrogate tumor markers, such as polynucleotide expression, can also be used to better classify, and thus diagnose and treat, different forms and disease states of cancer. Two classifications widely used in oncology that can benefit from identification of the expression levels of the polynucleotides of the invention are staging of the cancerous disorder, and grading the nature of the cancerous tissue.

Staging.

Staging is a process used by physicians to describe how advanced the cancerous state is in a patient. Staging assists the physician in determining a prognosis, planning treatment and evaluating the results of such treatment. Different staging systems are used for different types of cancer, but each generally involves the following determinations: the type of tumor, indicated by T; whether the cancer has metastasized to nearby lymph nodes, indicated by N; and whether the cancer has metastasized to more distant parts of the body, indicated by M. This system of staging is called the TNM system. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or another site, are called Stage IV, the most advanced stage.

Currently, the determination of staging is done using pathological techniques and is based more on the presence or absence of malignant tissue rather than the characteristics of the tumor type. Presence or absence of malignant tissue is based primarily on the gross morphology of the cells in the areas biopsied. The polynucleotides of the invention can facilitate fine-tuning of the staging process by identifying markers for the aggresivity of a cancer, e.g. the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

Grading of Cancers.

Grade is a term used to describe how closely a tumor resembles normal tissue of its same type. Based on the microscopic appearance of a tumor, pathologists will identify the grade of a tumor based on parameters such as cell morphology, cellular organization, and other markers of differentiation. As a general rule, the grade of a tumor corresponds to its rate of growth or aggressiveness. That is, undifferentiated or high-grade tumors grow more quickly than well differentiated or low-grade tumors. Information about tumor grade is useful in planning treatment and predicting prognosis.

The American Joint Commission on Cancer has recommended the following guidelines for grading tumors: 1) GX Grade cannot be assessed; 2) G1 Well differentiated; G2 Moderately well differentiated; 3) G3 Poorly differentiated; 4) G4 Undifferentiated. Although grading is used by pathologists to describe most cancers, it plays a more important role in treatment planning for certain types than for others. An example is the Gleason system that is specific for prostate cancer, which uses grade numbers to describe the degree of differentiation. Lower Gleason scores indicate well-differentiated cells. Intermediate scores denote tumors with moderately differentiated cells. Higher scores describe poorly differentiated cells. Grade is also important in some types of brain tumors and soft tissue sarcomas.

The polynucleotides of the invention can be especially valuable in determining the grade of the tumor, as they not only can aid in determining the differentiation status of the cells of a tumor, they can also identify factors other than differentiation that are valuable in determining the aggressivity of a tumor, such as metastatic potential.

Familial Cancer Genes.

A number of cancer syndromes are linked to Mendelian inheritance of a predisposition to develop particular cancers. The following table contains a list of cancer types that can be inherited, and for which the gene or genes responsible have been identified. Most of the cancer types listed can occur as part of several different genetic conditions, each caused by alterations in a different gene.

| Cancer Type | Genetic Condition | Gene |
|---|---|---|
| Brain | Li-Fraumeni syndrome | TP53 |
| | Neurofibromatosis 1 | NF1 |
| | Neurofibromatosis 2 | NF2 |
| | von Hippel-Lindau syndrome | VHL |
| | Tuberous sclerosis 2 | TSC2 |
| Breast | Hereditary breast/ovarian cancer 1 | BRCA1 |
| | Hereditary breast/ovarian cancer 2 | BRCA2 |
| | Li-Fraumeni syndrome | TP53 |
| | Ataxia telangiectasia | ATM |
| Colon | Familial adenomatous polyposis (FAP) | APC |
| | Hereditary non-polyposis colon cancer (HNPCC) 1 | HMSH2 |
| | Hereditary non-polyposis colon cancer (HNPCC) 2 | hMLH1 |
| | Hereditary non-polyposis colon cancer (HNPCC) 3 | hPMS1 |
| | Hereditary non-polyposis colon cancer (HNPCC) 4 | hPMS2 |
| Endocrine | Multiple endocrine neoplasia 1 (MEN1) | MEN1 |
| (parathyroid, pituitary, GI endocrine) | | |
| Endocrine | Multiple endocrine neoplasia 2 (MEN2) | RET |
| (pheochromacytoma, medullary thyroid) | | |
| Endometrial | Hereditary non-polyposis colon cancer (HNPCC) 1 | hMSH2 |
| | Hereditary non-polyposis colon cancer (HNPCC) 2 | hMLH1 |
| | Hereditary non-polyposis colon cancer (HNPCC) 3 | hPMS1 |
| | Hereditary non-polyposis colon cancer (HNPCC) 4 | hPMS2 |
| Eye | Hereditary retinoblastoma | RB1 |
| Hematologic | Li-Fraumeni syndrome | TP53 |
| (lymphomas and leukemia) | Ataxia telangiectasia | ATM |
| Kidney | Hereditary Wilms' tumor | WT1 |
| | von Hippel-Lindau syndrome | VHL |
| | Tuberous sclerosis 2 | TSC2 |
| Ovary | Hereditary breast/ovarian cancer 1 | BRCA1 |
| | Hereditary breast/ovarian cancer 2 | BRCA2 |
| Sarcoma | Hereditary retinoblastoma | RB1 |
| | Li-Fraumeni syndrome | TP53 |
| | Neurofibromatosis 1 | NF1 |
| Skin | Hereditary melanoma 1 | CDKN2 |
| | Hereditary melanoma 2 | CDK4 |
| | Basal cell naevus (Gorlin) syndrome | PTCH |
| Stomach | Hereditary non-polyposis colon cancer (HNPCC) 1 | hMSH2 |
| | Hereditary non-polyposis colon cancer (HNPCC) 2 | hMLH1 |
| | Hereditary non-polyposis colon cancer (HNPCC) 3 | hPMS1 |
| | Hereditary non-polyposis colon cancer (HNPCC) 4 | hPMS2 |

The polynucleotides of the invention can be especially useful to monitor patients having any of the above syndromes to detect potentially malignant events at a molecular level before they are detectable at a gross morphological level. As can be seen from the table, a number of genes are involved in multiple forms of cancer. Thus, a polynucleotide of the invention identified as important for metastatic colon cancer can also have clinical implications for a patient diagnosed with stomach cancer or endometrial cancer.

Lung Cancer.

Lung cancer is one of the most common cancers in the United States, accounting for about 15 percent of all cancer cases, or 170,000 new cases each year. At this time, over half of the lung cancer cases in the United States are in men, but the number found in women is increasing and will soon equal that in men. Today more women die of lung cancer than of breast cancer. Lung cancer is especially difficult to diagnose and treat because of the large size of the lungs, which allows cancer to develop for years undetected. In fact, lung cancer can spread outside the lungs without causing any symptoms. Adding to the confusion, the most common symptom of lung cancer, a persistent cough, can often be mistaken for a cold or bronchitis.

Although there are more than a dozen different kinds of lung cancer, the two main types of lung cancer are small cell and nonsmall cell, which encompass about 90% of all lung cancer cases. Small cell carcinoma (also called oat cell carcinoma), which usually starts in one of the larger bronchial tubes, grows fairly rapidly, and is likely to be large by the time of diagnosis. Nonsmall cell lung cancer (NSCLC) is made up of three general subtypes of lung cancer. Epidermoid carcinoma (also called squamous cell carcinoma) usually starts in one of the larger bronchial tubes and grows relatively slowly. The size of these tumors can range from very small to quite large. Adenocarcinoma starts growing near the outside surface of the lung and can vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

Currently, CT scans, MRIs, X-rays, sputum cytology, and biopsies are used to diagnose nonsmall cell lung cancer. The form and cellular origin of the lung cancer is diagnosed primarily through biopsy from either a surgical biopsy or a needle aspiration of lung tissue, and usually the biopsy is prompted from an abnormality identified on an X-ray. In some cases, sputum cytology can reveal lung cancers in patients with normal X-rays or can determine the type of lung cancer, but because it cannot pinpoint the tumor's location, a positive sputum cytology test is usually followed by further tests. Since these tests are based in large part on gross morphology of the tissue, the diagnosis of a particular kind of tumor is largely subjective, and the diagnosis can vary significantly between clinicians.

The polynucleotides of the invention can be used to distinguish types of lung cancer as well as identifying traits specific to a certain patient's cancer. For example, if the patient's biopsy expresses a polynucleotide that is associated with a low metastatic potential, it may justify leaving a larger portion of the patient's lung in surgery to remove the lesion. Alternatively, a smaller lesion with expression of a polynucleotide that is associated with high metastatic potential may justify a more radical removal of lung tissue and/or the surrounding lymph nodes, even if no metastasis can be identified through pathological examination.

Similarly, the expression of polynucleotides of the invention can be used in the diagnosis, prognosis and management of colorectal cancer. The differential expression of a polynucleotide in hyperplasia can be used as a diagnostic marker for metastatic lung cancer. The polynucleotides of the invention that would be especially useful for this purpose are those that exhibit differential expression between high metastatic versus low metastatic lung cancer, i.e. SEQ ID NOS: 9, 34, 42, 62, 74, 106, 119, 135, 154, 160, 260, 308, 323, 349, 361, 369, 371, 381, 395, and 400. Detection of malignant lung cancer with a higher metastatic potential can be determined using expression levels of any of these sequences alone or in combination with the levels of expression of other known genes.

Breast Cancer.

The National Cancer Institute (NCI) estimates that about 1 in 8 women in the United States will develop breast cancer during her lifetime. Clinical breast examination and mammography are recommended as combined modalities for breast cancer screening, and the nature of the cancer will often depend upon the location of the tumor and the cell type from which the tumor is derived. The majority of breast cancers are adenocarcinomas subtypes, which can be summarized as follows:

Ductal carcinoma in situ (DCIS): Ductal carcinoma in situ is the most common type of noninvasive breast cancer. In DCIS, the malignant cells have not metastasized through the walls of the ducts into the fatty tissue of the breast. Comedocarcinoma is a type of DCIS that is more likely than other types of DCIS to come back in the same area after lumpectomy. It is more closely linked to eventual development of invasive ductal carcinoma than other forms of DCIS.

Infiltrating (or invasive) ductal carcinoma (IDC): this type of cancer has metastasized through the wall of the duct and invaded the fatty tissue of the breast. At this point, it has the potential to use the lymphatic system and bloodstream for metastasis to more distant parts of the body. Infiltrating ductal carcinoma accounts for about 80% of breast cancers.

Lobular carcinoma in situ (LCIS): While not a true cancer, LCIS (also called lobular neoplasia) is sometimes classified as a type of noninvasive breast cancer. It does not penetrate through the wall of the lobules. Although it does not itself usually become an invasive cancer, women with this condition have a higher risk of developing an invasive breast cancer in the same breast, or in the opposite breast.

Infiltrating (or invasive) lobular carcinoma (ILC): ILC is similar to IDC, in that it has the potential metastasize elsewhere in the body. About 10% to 15% of invasive breast cancers are invasive lobular carcinomas. ILC can be more difficult to detect by mammogram than IDC.

Inflammatory breast cancer: This rare type of invasive breast cancer accounts for about 1% of all breast cancers and is extremely aggressive. Multiple skin symptoms associated with this cancer are caused by cancer cells blocking lymph vessels or channels in the skin over the breast.

Medullary carcinoma: This special type of infiltrating breast cancer has a relatively well defined, distinct boundary between tumor tissue and normal tissue. It accounts for about 5% of breast cancers. The prognosis for this kind of breast cancer is better than for other types of invasive breast cancer.

Mucinous carcinoma: This rare type of invasive breast cancer originates from mucus-producing cells. The prognosis for mucinous carcinoma is better than for the more common types of invasive breast cancer.

Paget's disease of the nipple: This type of breast cancer starts in the ducts and spreads to the skin of the nipple and the areola. It is a rare type of breast cancer, occurring in only 1% of all cases. Paget's disease can be associated with in situ carcinoma, or with infiltrating breast carcinoma. If no lump can be felt in the breast tissue, and the biopsy shows DCIS but no invasive cancer, the prognosis is excellent.

Phyllodes tumor: This very rare type of breast tumor forms from the stroma of the breast, in contrast to carcinomas which develop in the ducts or lobules. Phyllodes (also spelled phylloides) tumors are usually benign, but are malignant on rare occasions. Nevertheless, malignant phyllodes tumors are very rare and less than 10 women per year in the US die of this disease. Benign phyllodes tumors are successfully treated by removing the mass and a narrow margin of normal breast tissue.

Tubular carcinoma: Accounting for about 2% of all breast cancers, tubular carcinomas are a special type of infiltrating breast carcinoma. They have a better prognosis than usual infiltrating ductal or lobularcarcinomas.

High-quality mammography combined with clinical breast exam remains the only screening method clearly tied to reduction in breast cancer mortality. Lower dose x-rays, digitized computer rather than film images, and the use of computer programs to assist diagnosis, are almost ready for widespread dissemination. Other technologies also are being developed, including magnetic resonance imaging and ultrasound. In addition, a very low radiation exposure technique, positron emission tomography has the potential for detecting early breast cancer.

It is also possible to differentiate between non-cancerous breast tissue and malignant breast tissue by analyzing differential gene expression between tissues. In addition, there may be several possible alterations that lead to the various possible types of breast cancer. The different types of breast tumors (e.g., invasive vs. non-invasive, ductal vs. axillary lymph node) can be differentiable from one another by the identification of the differences in genes expressed by different types of breast tumor tissues (Porter-Jordan et al., Hematol Oncol Clin North Am (1994) 8:73). Breast cancer can thus be generally diagnosed by detection of expression of a gene or genes associated with breast tumors. Where enough information is available about the differential gene expression between various types of breast tumor tissues, the specific type of breast tumor can also be diagnosed.

For example, increased estrogen receptor (ER) expression in normal breast epithileum, while not itself indicative of malignant tissue, is a known risk marker for development of breast cancer. Khan S A et al., Cancer Res (1994) 54:993. Malignant breast cancer is often divided into two groups, ER-positive and ER-negative, based on the estrogen receptor status of the tissue. The ER status represents different survival length and response to hormone therapy, and is thought to represent either: 1) an indicator of different stages of the disease, or 2) an indicator that allows differentiation between two similar but distinct diseases. K. Zhu et al., Med. Hypoth. (1997) 49:69. A number of other genes are known to vary expression between either different stages of cancer or different types of similar breast cancer.

Similarly, the expression of polynucleotides of the invention can be used in the diagnosis and management of breast cancer. The differential expression of a polynucleotide in human breast tumor tissue can be used as a diagnostic marker for human breast cancer. The polynucleotides of the invention that would be especially useful for this purpose are those that exhibit differential expression between breast cancer tissue with a high metastatic potential and a low metastatic potential, ie. SEQ ID NOS: 9, 42, 52, 62, 65, 66, 68, 114, 123, 144, 172, 178, 214, 219, 223, 258, 317, and 379. Detection of breast cancer can be determined using expression levels of any of these sequences alone or in combination. Determination of the aggressive nature and/or the metastatic potential of a breast cancer can also be determined by comparing levels of one or more polynucleotides of the invention and comparing levels of another sequence known to vary in cancerous tissue, e.g. ER expression. In addition, development of breast cancer can be detected by examining the ratio of SEQ ID NO: to the levels of steroid hormones (e.g., testosterone or estrogen) or to other hormones (e.g., growth hormone, insulin). Thus expression of specific marker polynucleotides can be used to discriminate between normal and cancerous breast tissue, to discriminate between breast cancers with different cells of origin, to discriminate between breast cancers with different potential metastatic rates, etc.

Diagnosis of breast cancer can also involve comparing the expression of a polynucleotide of the invention with the expression of other sequences in non-malignant breast tissue samples in comparison to one or more forms of the diseased tissue. A comparison of expression of one or more polynucleotides of the invention between the samples provides information on relative levels of these polynucleotides as well as the ratio of these polynucleotides to the expression of other sequences in the tissue of interest compared to normal.

This risk of breast cancer is elevated significantly by the presence of an inherited risk for breast cancer, such as a mutation in BRCA-1 or BRCA-2. New diagnostic tools are being developed to address the needs of higher risk patients to complement mammography and physical examinations for early detection of breast cancer, particularly among younger women. The presence of antigen or expression markers in nipple aspirate fluid (NAF) samples collected from one or both breasts can be useful for useful for risk assessment or early cancer detection. Breast cytology and biomarkers obtained by random fine needle aspiration have been used to identify hyperplasia with atypia and overexpression of p53 and EGFR. The polynucleotides of the invention can be used in multivariate analysis with expression studies with genes such as p53 and EGFR as risk predictors and as surrogate endpoint biomarkers for breast cancer.

As well as being used for diagnosis and risk assessment, the expression of certain genes can also correlated to prognosis of a disease state. The expression of particular gene have been used as prognostic indicators for breast cancer including increased expression of c-erbB-2, pS2, ER, progesterone receptor, epidermal growth factor receptor (EGFR), neu, myc, bcl-2, int2, cytosolic tyrosine kinase, cyclin E, prad-1, hst, uPA, PAI-1, PAI-2, cathepsin D, as well as the presence of a number of cancer-specific antigens, e.g. CEA, CA M26, CA M29 and CA 15.3. Davis, Br. J. Biomed Sci. (1996) 53:157. Poor prognosis has also been linked to a decrease in expression of certain genes, such as pS3, Rb, nm23. The expression of the polynucleotides of the invention can be of prognostic value for determining the metastatic potential of a malignant breast cancer, as this molecules are differentially expressed between high and low metastatic potential tissues tumors. The levels of these polynucleotides in patients with malignant breast cancer can compared to normal tissue, malignant tissue with a known high potential metastatic level, and malignant tissue with a known lower level of metastatic potential to provide a prognosis for a particular patient. Such a prognosis is predictive of the extent and nature of the cancer. The determined prognosis is useful in determining the prognosis of a patient with breast cancer, both for initial treatment of the disease and for longer-term monitoring of the same patient. If samples are taken from the same individual over a period of time, differences in polynucleotide expression that are specific to that patient can be identified and closely watched.

Colon Cancer.

Colorectal cancer is one of the most common neoplasms in humans and perhaps the most frequent form of hereditary neoplasia. Prevention and early detection are key factors in controlling and curing colorectal cancer. Indeed, colorectal cancer is the second most preventable cancer, after lung cancer. Colorectal cancer begins as polyps, which are small, benign growths of cells that form on the inner lining of the colon. Over a period of several years, some of these polyps accumulate additional mutations and become cancerous. About 20 percent of all cases of colon cancer are thought to be related to heredity. Currently, multiple familial colorectal cancer disorders have been identified, which are summarized as follows:

Familial adenomatous polyposis (FAP): This condition results in a person having hundreds or even thousands of polyps in the colon and rectum that usually first appear during the teenage years. Cancer nearly always develops in one or more of these polyps between the ages of 30 and 50.

Gardner's syndrome: Like FAP, Gardner's syndrome results in polyps and colorectal cancers that develop at a young age. It can also cause benign tumors of the skin, soft connective tissue and bones.

Hereditary nonpolyposis colon cancer (HNPCC): People with this condition tend to develop colorectal cancer at a young age, without first having many polyps. HNPCC has an autosomal dominant pattern of inheritance with variable but high penetrance estimated to be about 90%. HNPCC underlies 0.5%-10% of all cases of colorectal cancer. An understanding of the mechanisms behind the development of HNPCC is emerging, and genetic presymptomatic testing, now being conducted in research settings, soon will be available on a widespread basis for individuals identified at risk for this disease.

Familial colorectal cancer in Ashkenazi Jews: Recent research has found an inherited tendency to developing colorectal cancer among some Jews of Eastern European descent. Like people with FAP, Gardner's syndrome, and HNPCC, their increased risk is due to an inherited mutation present in about 6% of American Jews.

Several tests are currently used to screen for colorectal cancer, including digital rectal examination, fecal occult blood test, sigmoidoscopy, colonoscopy, virtual colonoscopy and MRI. Each of these tests identifies potential colorectal cancer lesions, or a risk of development of these lesions, at a fairly gross morphological level.

The sequential alteration of a number of genes is associated with malignant adenocarcinoma, including the genes DCC, p53, ras, and FAP. For a review, see e.g. Fearon E R, et al., Cell (1990) 61(5):759; Hamilton S R et al., Cancer (1993) 72:957; Bodmer W, et al., Nat Genet. (1994) 4(3):217; Fearon E R, Ann NY Acad Sci. (1995) 768:101. Molecular genetic alterations are thus promising as potential diagnostic and prognostic indicators in colorectal carcinoma and molecular genetics of colorectal carcinoma since it is possible to differentiate between different types of colorectal neoplasias using molecular markers. Colorectal cancer can thus be generally diagnosed by detection of expression of a gene or genes associated with colorectal tumors.

Similarly, the expression of polynucleotides of the invention can be used in the diagnosis, prognosis and management of colorectal cancer. The differential expression of a polynucleotide in hyperplasia can be used as a diagnostic marker for colon cancer. The polynucleotides of the invention that would be especially useful for this purpose are those that exhibit differential expression between malignant metastatic colon cancer and normal patient tissue, i.e. SEQ ID NOS: 52, 119, 172, 288. Detection of malignant colon cancer can be determined using expression levels of any of these sequences alone or in combination with the levels of expression.

Determination of the aggressive nature and/or the metastatic potential of a colon cancer can also be determined by comparing levels of one or more polynucleotides of the invention and comparing total levels of another sequence known to vary in cancerous tissue, e.g. p53 expression. In addition, development of colon cancer can be detected by examining the ratio of any of the polynucleotides of the invention to the levels of oncogenes (e.g. ras) or tumor suppressor genes (e.g. FAP or p53). Thus expression of specific marker polynucleotides can be used to discriminate between normal and cancerous breast tissue, to discriminate between breast cancers with different cells of origin, to discriminate between breast cancers with different potential metastatic rates, etc.

G. Use of Polynucleotides to Screen for Peptide Analogs and Antagonists

Polypeptides encoded by the instant polynucleotides and corresponding full length genes can be used to screen peptide libraries to identify binding partners, such as receptors, from among the encoded polypeptides.

A library of peptides can be synthesized following the methods disclosed in U.S. Pat. No. 5,010,175 ('175), and in WO 91/17823. As described below in brief, one prepares a mixture of peptides, which is then screened to identify the peptides exhibiting the desired signal transduction and receptor binding activity. In the '175 method, a suitable peptide synthesis support (e.g., a resin) is coupled to a mixture of appropriately protected, activated amino acids. The concentration of each amino acid in the reaction mixture is balanced or adjusted in inverse proportion to its coupling reaction rate so that the product is an equimolar mixture of amino acids coupled to the starting resin. The bound amino acids are then deprotected, and reacted with another balanced amino acid mixture to form an equimolar mixture of all possible dipeptides. This process is repeated until a mixture of peptides of the desired length (e.g., hexamers) is formed. Note that one need not include all amino acids in each step: one can include only one or two amino acids in some steps (e.g., where it is known that a particular amino acid is essential in a given position), thus reducing the complexity of the mixture. After the synthesis of the peptide library is completed, the mixture of peptides is screened for binding to the selected polypeptide. The peptides are then tested for their ability to inhibit or enhance activity. Peptides exhibiting the desired activity are then isolated and sequenced. The method described in WO 91/17823 is similar. However, instead of reacting the synthesis resin with a mixture of activated amino acids, the resin is divided into twenty equal portions (or into a number of portions corresponding to the number of different amino acids to be added in that step), and each amino acid is coupled individually to its portion of resin. The resin portions are then combined, mixed, and again divided into a number of equal portions for reaction with the second amino acid. In this manner, each reaction can be easily driven to completion. Additionally, one can maintain separate “subpools” by treating portions in parallel, rather than combining all resins at each step. This simplifies the process of determining which peptides are responsible for any observed receptor binding or signal transduction activity.

In such cases, the subpools containing, e.g., 1-2,000 candidates each are exposed to one or more polypeptides of the invention. Each subpool that produces a positive result is then resynthesized as a group of smaller subpools (sub-subpools) containing, e.g., 20-100 candidates, and reassayed. Positive sub-subpools can be resynthesized as individual compounds, and assayed finally to determine the peptides that exhibit a high binding constant. These peptides can be tested for their ability to inhibit or enhance the native activity. The methods described in WO 91/7823 and U.S. Pat. No. 5,194,392 (herein incorporated by reference) enable the preparation of such pools and subpools by automated techniques in parallel, such that all synthesis and resynthesis can be performed in a matter of days.

Peptide agonists or antagonists are screened using any available method, such as signal transduction, antibody binding, receptor binding, mitogenic assays, chemotaxis assays, etc. The methods described herein are presently preferred. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide can require concentrations equal to or greater than the native concentration, while inhibitors capable of binding irreversibly to the polypeptide can be added in concentrations on the order of the native concentration.

The end results of such screening and experimentation will be at least one novel polypeptide binding partner, such as a receptor, encoded by a gene or a cDNA corresponding to a polynucleotide of the invention, and at least one peptide agonist or antagonist of the novel binding partner. Such agonists and antagonists can be used to modulate, enhance, or inhibit receptor function in cells to which the receptor is native, or in cells that possess the receptor as a result of genetic engineering. Further, if the novel receptor shares biologically important characteristics with a known receptor, information about agonist/antagonist binding can facilitate development of improved agonists/antagonists of the known receptor.

H. Pharmaceutical Compositions and Therapeutic Uses

Pharmaceutical compositions can comprise polypeptides, antibodies, or polynucleotides of the claimed invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier.

Delivery Methods.

Once formulated, the compositions of the invention can be (1) administered directly to the subject (e.g., as polynucleotide or polypeptides); (2) delivered ex vivo, to cells derived from the subject (e.g., as in ex vivo gene therapy); or (3) delivered in vitro for expression of recombinant proteins (e.g., polynucleotides). Direct delivery of the compositions will generally be accomplished by injection, either subcutaneously, intraperitoneally, intravenously or intramuscularly, or delivered to the interstitial space of a tissue. The compositions can also be administered into a tumor or lesion. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Once a gene corresponding to a polynucleotide of the invention has been found to correlate with a proliferative disorder, such as neoplasia, dysplasia, and hyperplasia, the disorder can be amenable to treatment by administration of a therapeutic agent based on the provided polynucleotide or corresponding polypeptide.

Preparation of antisense polynucleotides is discussed above. Neoplasias that are treated with the antisense composition include, but are not limited to, cervical cancers, melanomas, colorectal adenocarcinomas, Wilms' tumor, retinoblastoma, sarcomas, myosarcomas, lung carcinomas, leukemias, such as chronic myelogenous leukemia, promyelocytic leukemia, monocytic leukemia, and myeloid leukemia, and lymphomas, such as histiocytic lymphoma. Proliferative disorders that are treated with the therapeutic composition include disorders such as anhydric hereditary ectodermal dysplasia, congenital alveolar dysplasia, epithelial dysplasia of the cervix, fibrous dysplasia of bone, and mammary dysplasia. Hyperplasias, for example, endometrial, adrenal, breast, prostate, or thyroid hyperplasias or pseudoepitheliomatous hyperplasia of the skin, are treated with antisense therapeutic compositions based upon a polynucleotide of the invention. Even in disorders in which mutations in the corresponding gene are not implicated, downregulation or inhibition of expression of a gene corresponding to a polynucleotide of the invention can have therapeutic application. For example, decreasing gene expression can help to suppress tumors in which enhanced expression of the gene is implicated.

Both the dose of the antisense composition and the means of administration are determined based on the specific qualities of the therapeutic composition, the condition, age, and weight of the patient, the progression of the disease, and other relevant factors. Administration of the therapeutic antisense agents of the invention includes local or systemic administration, including injection, oral administration, particle gun or catheterized administration, and topical administration. Preferably, the therapeutic antisense composition contains an expression construct comprising a promoter and a polynucleotide segment of at least 12, 22, 25, 30, or 35 contiguous nucleotides of the antisense strand of a polynucleotide disclosed herein. Within the expression construct, the polynucleotide segment is located downstream from the promoter, and transcription of the polynucleotide segment initiates at the promoter.

Various methods are used to administer the therapeutic composition directly to a specific site in the body. For example, a small metastatic lesion is located and the therapeutic composition injected several times in several different locations within the body of tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. The antisense composition is directly administered to the surface of the tumor, for example, by topical application of the composition. X-ray imaging is used to assist in certain of the above delivery methods.

Receptor-mediated targeted delivery of therapeutic compositions containing an antisense polynucleotide, subgenomic polynucleotides, or antibodies to specific tissues is also used. Receptor-mediated DNA delivery techniques are described in, for example, Findeis et al., Trends Biotechnol. (1993) 11:202; Chiou et al., Gene Therapeutics: Methods And Applications OfDirect Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Preferably, receptor-mediated targeted delivery of therapeutic compositions containing antibodies of the invention is used to deliver the antibodies to specific tissue.

Therapeutic compositions containing antisense subgenomic polynucleotides are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action and efficacy of transformation and expression are considerations which will affect the dosage required for ultimate efficacy of the antisense subgenomic polynucleotides. Where greater expression is desired over a larger area of tissue, larger amounts of antisense subgenomic polynucleotides or the same amounts readministered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect. A more complete description of gene therapy vectors, especially retroviral vectors, is contained in U.S. Ser. No. 08/869,309, which is expressly incorporated herein, and in section G below.

For polynucleotide-related genes encoding polypeptides or proteins with anti-inflammatory activity, suitable use, doses, and administration are described in U.S. Pat. No. 5,654,173. Therapeutic agents also include antibodies to proteins and polypeptides encoded by the polynucleotides of the invention and related genes, as described in U.S. Pat. No. 5,654,173.

I. Gene Therapy

The therapeutic polynucleotides and polypeptides of the present invention can be utilized in gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5, 219,740; WO 93/11230; WO 93/10218; Vile and Hart, Cancer Res. (1 993) 53:3 860; Vile et al., Cancer Res. (1 993) 53:962; Ram et al., Cancer Res. (1993) 53:83; Takamiya et al., J. Neurosci. Res. (1992) 33:493; Baba et al., J. Neurosurg. (1993) 79:729; U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs can be readily prepared (see, e.g., WO 95/30763 and WO 92/05266), and used to create producer cell lines (also termed vector cell lines) for the production of recombinant vector particles. Within particularly preferred embodiments of the invention, packaging cell lines are made from human (such as HTT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; WO 92/10578; WO 94/21792; WO 95/27069; WO 95/27044; and WO 95/07994. Gene delivery vehicles of the present invention can also employ parvovirus such as adeno-associated virus (AAV) vectors. Representative examples include the AAV vectors disclosed by Srivastava in WO 93/09239, Samulski et al., J. Virol. (1989) 63:3822; Mendelson et al., Virol. (1988)166:154; and Flotte et al., PNAS (1993) 90:10613.

Representative examples of adenoviral vectors include those described by Berkner, Biotechniques (1988) 6:616; Rosenfeld et al., Science (1991) 252:431; WO 93/19191; Kolls et al., PNAS (1994) 91:215; Kass-Eisler et al., PNAS (1993) 90:11498; Guzman et al., Circulation (1993) 88:2838; Guzman et al., Cir. Res. (1993) 73:1202; Zabner et al., Cell (1993) 75:207; Li et al., Hum. Gene Ther. (1993) 4:403; Cailaud et al., Eur. J. Neurosci. (1993) 5:1287; Vincent et al., Nat. Genet. (1993) 5:130; Jaffe et al., Nat. Genet. (1992) 1:372; and Levrero et al., Gene (1991) 101:195. Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. (1992)3:147 can be employed.

Other gene delivery vehicles and methods can be employed, including polycationic condensed DNA linked or unlinked to killed adenovirus alone, for example Curiel, Hum. Gene Ther. (1992) 3:147; ligand linked DNA, for example see Wu, J. Biol. Chem. (1989) 264:16985; eukaryotic cell delivery vehicles cells, for example see U.S. Pat. No. 5,814,482; WO 95/07994; WO 96/17072; WO 95/30763; and WO 97/42338; deposition of photopolymerized hydrogel materials; hand-held gene transfer particle gun, as described in U.S. Pat. No. 5,149,655; ionizing radiation as described in U.S. Pat. No. 5,206,152 and in WO92/11033; nucleic charge neutralization or fusion with cell membranes. Additional approaches are described in Philip, Mol. Cell Biol. (1994) 14:2411, and in Woffendin, Proc. Natl. Acad. Sci. (1994) 91:1581.

Naked DNA can also be employed. Exemplary naked DNA introduction methods are described in WO 90/11092 and U.S. Pat. No. 5,580,859. Uptake efficiency can be improved using biodegradable latex beads. DNA coated latex beads are efficiently transported into cells after endocytosis initiation by the beads. The method can be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120; WO 95/13796; WO 94/23697; WO 91/14445; and EP 0524968.

Further non-viral delivery suitable for use includes mechanical delivery systems such as the approach described in Woffendin et al., Proc. Natl. Acad Sci. USA (1994) 91(24):11581. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and WO 92/11033.

The present invention will now be illustrated by reference to the following examples which set forth particularly advantageous embodiments. However, it should be noted that these embodiments are illustrative and are not to be construed as restricting the invention in any way.

### EXAMPLES

The present invention is now illustrated by reference to the following examples which set forth particularly advantageous embodiments. However, these embodiments are illustrative and are not meant to be construed as restricting the invention in any way.

### Example 1

### Source of Biological Materials and Overview of Novel Polynucleotides Expressed by the Biological Materials

Human colon cancer cell line Km12L4-A (Morika, W. A. K. et al., Cancer Research (1988) 48:6863) was used to construct a cDNA library from mRNA isolated from the cells. As described in the above overview, a total of 4,693 sequences expressed by the Km12L4-A cell line were isolated and analyzed; most sequences were about 275-300 nucleotides in length. The KM12L4-A cell line is derived from the KM12C cell line. The KM12C cell line, which is poorly metastatic (low metastatic) was established in culture from a Dukes' stage B₂ surgical specimen (Morikawa et al. Cancer Res. (1988) 48:6863). The KML4-A is a highly metastatic subline derived from KM12C (Yeatman et al. Nucl. Acids. Res. (1995) 23:4007; Bao-Ling et al. Proc. Annu. Meet. Am. Assoc. Cancer. Res. (1995) 21:3269). The KM12C and KM12C-derived cell lines (e.g., KM12L4, KM12L4-A, etc.) are well-recognized in the art as a model cell line for the study of colon cancer (see, e.g., Moriakawa et al., supra; Radinsky et al. Clin. Cancer Res. (1995) 1:19; Yeatman et al., (1995) supra; Yeatman et al. Clin. Exp. Metastasis (1996) 14:246).

The sequences were first masked to eliminate low complexity sequences using the XBLAST masking program (Clayerie “Effective Large-Scale Sequence Similarity Searches,” In: Computer Methods for Macromolecular Sequence Analysis, Doolittle, ed., Meth. Enzymol. 266:212-227 Academic Press, NY, N.Y. (1996); see particularly Clayerie, in “Automated DNA Sequencing and Analysis Techniques” Adams et al., eds., Chap. 36, p. 267 Academic Press, San Diego, 1994 and Clayerie et al. Comput. Chem. (1993) 17:191). Generally, masking does not influence the final search results, except to eliminate of relative little interest due to their lox complexity, and to eliminate multiple “hits” based on similarity to repetitive regions common to multiple sequences, e.g., Alu repeats. Masking resulted in the elimination of 43 sequences. The remaining sequences were then used in a BLASTN vs. Genbank search with search parameters of greater than 70% overlap, 99% identity, and a p value of less than 1×10⁻⁴⁰, which search resulted in the discarding of 1,432 sequences. Sequences from this search also were discarded if the inclusive parameters were met, but the sequence was ribosomal or vector-derived.

The resulting sequences from the previous search were classified into three groups (1, 2 and 3 below) and searched in a BLASTX vs. NRP (non-redundant proteins) database search: (1) unknown (no hits in the Genbank search), (2) weak similarity (greater than 45% identity and p value of less than 1×10⁻⁵), and (3) high similarity (greater than 60% overlap, greater than 80% identity, and p value less than 1×10⁻⁵). This search resulted in discard of 98 sequences as having greater than 70% overlap, greater than 99% identity, and p value of less than 1×10₋₄₀.

The remaining sequences were classified as unknown (no hits), weak similarity, and high similarity (parameters as above). Two searches were performed on these sequences. First, a BLAST vs. EST database search resulted in discard of 1771 sequences (sequences with greater than 99% overlap, greater than 99% similarity and a p value of less than 1×10⁻⁴⁰; sequences with a p value of less than 1×10⁻⁶⁵ when compared to a database sequence of human origin were also excluded). Second, a BLASTN vs. Patent GeneSeq database resulted in discard of 15 sequences (greater than 99% identity; p value less than 1×10⁻⁴⁰; greater than 99% overlap).

The remaining sequences were subjected to screening using other rules and redundancies in the dataset. Sequences with a p value of less than 1×10⁻¹¹¹ in relation to a database sequence of human origin were specifically excluded. The final result provided the 404 sequences listed in the accompanying Sequence Listing. The Sequence Listing is arranged beginning with sequences with no similarity to any sequence in a database searched, and ending with sequences with the greatest similarity. Each identified polynucleotide represents sequence from at least a partial mRNA transcript. Polynucleotides that were determined to be novel were assigned a sequence identification number.

The novel polynucleotides and were assigned sequence identification numbers SEQ ID NOS: 1-404. The DNA sequences corresponding to the novel polynucleotides are provided in the Sequence Listing. The majority of the sequences are presented in the Sequence Listing in the 5′ to 3′ direction. A small number, 25, are listed in the Sequence Listing in the 5′ to 3′ direction but the sequence as written is actually 3′ to 5′. These sequences are readily identified with the designation “AR” in the Sequence Name in Table 1 (inserted before the claims). The sequences correctly listed in the 5′ to 3′ direction in the Sequence Listing are designated “AF.” The Sequence Listing filed herewith therefore contains 25 sequences listed in the reverse order, namely SEQ ID NOS:47, 97, 137, 171, 173, 179, 182, 194, 200, 202, 213, 227, 258, 264, 275, 302, 313, 324, 329, 330, 331, 338, 358, 379, and 404.

Because the provided polynucleotides represent partial mRNA transcripts, two or more polynucleotides of the invention may represent different regions of the same mRNA transcript and the same gene. Thus, if two or more SEQ ID NOS: are identified as belonging to the same clone, then either sequence can be used to obtain the full-length mRNA or gene.

In order to confirm the sequences of SEQ ID NOS:1-404, inserts of the clones corresponding to these polynucleotides were re-sequenced. These “validation” sequences are provided in SEQ ID NOS:405-800. These validation sequences were often longer than the original polynucleotide sequences. They validate, and thus often provide additional sequence information. Validation sequences can be correlated with the original sequences they validate by identifying those sequences of SEQ ID NOS:1-404 and the validation sequences of SEQ ID NOS:405-800 that share the same clone name in Table 1.

### Example 2

### Results of Public Database Search to Identify Function of Gene Products

SEQ ID NOS:1-404, as well as the validation sequences SEQ ID NOS:405-800, were translated in all three reading frames to determine the best alignment with the individual sequences. These amino acid sequences and nucleotide sequences are referred, generally, as query sequences, which are aligned with the individual sequences. Query and individual sequences were aligned using the BLAST programs, available over the world wide web at http://ww.ncbi.nlm.nih.gov/BLAST/. Again the sequences were masked to various extents to prevent searching of repetitive sequences or poly-A sequences, using the XBLAST program for masking low complexity as described above in Example 1.

Table 2 (inserted before the claims) shows the results of the alignments. Table 2 refers to each sequence by its SEQ ID NO:, the accession numbers and descriptions of nearest neighbors from the Genbank and Non-Redundant Protein searches, and the p values of the search results. Table 1 identifies each SEQ ID NO: by SEQ name, clone ID, and cluster. As discussed above, a single cluster includes polynucleotides representing the same gene or gene family, and generally represents sequences encoding the same gene product.

For each of SEQ ID NOS:1-800, the best alignment to a protein or DNA sequence is included in Table 2. The activity of the polypeptide encoded by SEQ ID NOS:1-800 is the same or similar to the nearest neighbor reported in Table 2. The accession number of the nearest neighbor is reported, providing a reference to the activities exhibited by the nearest neighbor. The search program and database used for the alignment also are indicated as well as a calculation of the p value.

Full length sequences or fragments of the polynucleotide sequences of the nearest neighbors can be used as probes and primers to identify and isolate the full length sequence of SEQ ID NOS:1-800. The nearest neighbors can indicate a tissue or cell type to be used to construct a library for the full-length sequences of SEQ ID NOS:1-800.

SEQ ID NOS:1-800 and the translations thereof may be human homologs of known genes of other species or novel allelic variants of known human genes. In such cases, these new human sequences are suitable as diagnostics or therapeutics. As diagnostics, the human sequences SEQ ID NOS:1-800 exhibit greater specificity in detecting and differentiating human cell lines and types than homologs of other species. The human polypeptides encoded by SEQ ID NOS:1-800 are likely to be less immunogenic when administered to humans than homologs from other species. Further, on administration to humans, the polypeptides encoded by SEQ ID NOS:1-800 can show greater specificity or can be better regulated by other human proteins than are homologs from other species.

### Example 3

### Members of Protein Families

After conducting a profile search as described in the specification above, several of the polynucleotides of the invention were found to encode polypeptides having characteristics of a polypeptide belonging to a known protein families (and thus represent new members of these protein families) and/or comprising a known functional domain (Table 3). Thus the invention encompasses fragments, fusions, and variants of such polynucleotides that retain biological activity associated with the protein family and/or functional domain identified herein.

TABLE 3
Polynucleotides encoding gene products of a protein family or having a known
functional domain(s).

| SEQ ID | | | | |
|---|---|---|---|---|
| NO: | Biological Activity (Profile hit) | Start | Stop | Dir |
| 24 | 4 transmembrane segments integral membrane proteins | 1218 | 578 | rev |
| 41 | 4 transmembrane segments integral membrane proteins | 1086 | 413 | rev |
| 101 | 4 transmembrane segments integral membrane proteins | 1206 | 544 | rev |
| 157 | 4 transmembrane segments integral membrane proteins | 721 | 33 | rev |
| 341 | 4 transmembrane segments integral membrane proteins | 1253 | 613 | rev |
| 395 | 4 transmembrane segments integral membrane proteins | 530 | 10 | for |
| 395 | 4 transmembrane segments integral membrane proteins | 696 | 17 | for |
| 395 | 4 transmembrane segments integral membrane proteins | 471 | 39 | rev |
| 24 | 7 transmembrane receptor (Secretin family) | 1301 | 491 | rev |
| 41 | 7 transmembrane receptor (Secretin family) | 1309 | 10 | rev |
| 101 | 7 transmembrane receptor (Secretin family) | 1330 | 296 | rev |
| 157 | 7 transmembrane receptor (Secretin family) | 1173 | 249 | rev |
| 291 | 7 transmembrane receptor (Secretin family) | 1400 | 269 | rev |
| 291 | 7 transmembrane receptor (Secretin family) | 712 | 130 | for |
| 305 | 7 transmembrane receptor (Secretin family) | 926 | 4 | for |
| 305 | 7 transmembrane receptor (Secretin family) | 753 | 55 | rev |
| 315 | 7 transmembrane receptor (Secretin family) | 1058 | 270 | rev |
| 341 | 7 transmembrane receptor (Secretin family) | 1265 | 534 | rev |
| 116 | Ank repeat | 141 | 218 | for |
| 251 | Ank repeat | 290 | 207 | for |
| 251 | Ank repeat | 467 | 387 | for |
| 63 | ATPases Associated with Various Cellular Activities | 543 | 60 | for |
| 116 | ATPases Associated with Various Cellular Activities | 802 | 313 | for |
| 134 | ATPases Associated with Various Cellular Activities | 525 | 57 | rev |
| 136 | ATPases Associated with Various Cellular Activities | 712 | 163 | for |
| 151 | ATPases Associated with Various Cellular Activities | 719 | 73 | for |
| 151 | ATPases Associated with Various Cellular Activities | 386 | 13 | for |
| 384 | ATPases Associated with Various Cellular Activities | 664 | 140 | for |
| 404 | ATPases Associated with Various Cellular Activities | 704 | 52 | for |
| 374 | Basic region plus leucine zipper transcription factors | 298 | 146 | for |
| 97 | Bromodomain (conserved sequence found in human, | 230 | 63 | for |
| | Drosophila and yeast proteins.) | | | |
| 136 | EF-hand | 121 | 207 | for |
| 242 | EF-hand | 238 | 155 | for |
| 379 | EF-hand | 212 | 126 | for |
| 308 | Eukaryotic aspartyl proteases | 1300 | 461 | rev |
| 213 | GATA family of transcription factors | 720 | 377 | for |
| 367 | G-protein alpha subunit | 971 | 467 | rev |
| 188 | Phorbol esters/diacylglycerol binding | 91 | 177 | for |
| 251 | Phorbol esters/diacylglycerol binding | 133 | 219 | for |
| 202 | protein kinase | 482 | 1 | rev |
| 202 | protein kinase | 970 | 1 | rev |
| 315 | protein kinase | 739 | 158 | for |
| 315 | protein kinase | 1023 | 197 | for |
| 367 | protein kinase | 1046 | 285 | rev |
| 397 | protein kinase | 511 | 6 | for |
| 256 | Protein phosphatase 2C | 13 | 90 | for |
| 256 | Protein phosphatase 2C | 163 | 86 | for |
| 382 | Protein Tyrosine Phosphatase | 261 | 2 | for |
| 306 | SH3 Domain | 141 | 296 | for |
| 386 | SH3 Domain | 359 | 209 | for |
| 169 | Trypsin | 764 | 164 | rev |
| 188 | WD domain, G-beta repeats | 480 | 382 | for |
| 188 | WD domain, G-beta repeats | 206 | 117 | for |
| 335 | WD domain, G-beta repeats | 3 | 92 | for |
| 23 | wnt family of developmental signaling proteins | 1151 | 335 | rev |
| 291 | wnt family of developmental signaling proteins | 779 | 89 | rev |
| 291 | wnt family of developmental signaling proteins | 1347 | 382 | rev |
| 324 | wnt family of developmental signaling proteins | 1180 | 499 | rev |
| 330 | wnt family of developmental signaling proteins | 1180 | 499 | rev |
| 341 | wnt family of developmental signaling proteins | 1399 | 560 | rev |
| 353 | wnt family of developmental signaling proteins | 880 | 49 | rev |
| 188 | WW/rsp5/WWP domain containing proteins | 431 | 354 | for |
| 379 | WW/rsp5/WWP domain containing proteins | 12 | 89 | for |
| 395 | WW/rsp5/WWP domain containing proteins | 153 | 76 | for |
| 395 | WW/rsp5/WWP domain containing proteins | 156 | 64 | for |
| 61 | Zinc finger, C2H2 type | 254 | 192 | for |
| 306 | Zinc finger, C2H2 type | 428 | 367 | for |
| 386 | Zinc finger, C2H2 type | 191 | 253 | for |
| 322 | Zinc finger, CCHC class | 553 | 503 | for |
| 306 | Zinc-binding metalloprotease domain | 101 | 60 | rev |
| 395 | Zinc-binding metalloprotease domain | 28 | 69 | rev |

Start and stop indicate the position within the individual sequenes that align with the query sequence having the indicated SEQ ID NO. The direction (Dir) indicates the orientation of the query sequence with respect to the individual sequence, where forward (for) indicates that the alignment is in the same direction (left to right) as the sequence provided in the Sequence Listing and reverse (rev) indicates that the alignment is with a sequence complementary to the sequence provided in the Sequence Listing.

Some polynucleotides exhibited multiple profile hits because, for example, the particular sequence contains overlapping profile regions, and/or the sequence contains two different functional domains. These profile hits are described in more detail below.

a) Four Transmembrane Integral Membrane Proteins.

SEQ ID NOS: 24, 41, 101, 157, 341, and 395 correspond to a sequence encoding a polypeptide that is a member of the 4 transmembrane segments integral membrane protein family (transmembrane 4 family). The transmembrane 4 family of proteins includes a number of evolutionarily-related eukaryotic cell surface antigens (Levy et al., J. Biol. Chem., (1991) 266:14597; Tomlinson et al., Eur. J. Immunol. (1993) 23:136; Barclay et al. The leucocyte antigen factbooks. (1993) Academic Press, London/San Diego). The proteins belonging to this family include: 1) Mammalian antigen CD9 (MIC3), which is involved in platelet activation and aggregation; 2) Mammalian leukocyte antigen CD37, expressed on B lymphocytes; 3) Mammalian leukocyte antigen CD53 (OX-44), which is implicated in growth regulation in hematopoietic cells; 4) Mammalian lysosomal membrane protein CD63 (melanoma-associated antigen ME491; antigen AD1); 5) Mammalian antigen CD81 (cell surface protein TAPA-1), which is implicated in regulation of lymphoma cell growth; 6) Mammalian antigen CD82 (protein R2; antigen C33; Kangai 1 (KAI1)), which associates with CD4 or CD8 and delivers costimulatory signals for the TCR/CD3 pathway; 7) Mammalian antigen CD151 (SFA-1; platelet-endothelial tetraspan antigen 3 (PETA-3)); 8) Mammalian cell surface glycoprotein A 15 (TALLA-1; MXS 1); 9) Mammalian novel antigen 2 (NAG-2); 10) Human tumor-associated antigen CO-029; 11) Schistosoma mansoni and japonicum 23 Kd surface antigen (SM23/SJ23).

The members of the 4 transmembrane family share several characteristics. First, they all are apparently type III membrane proteins, which are integral membrane proteins containing an N-terminal membrane-anchoring domain which is not cleaved during biosynthesis and which functions both as a translocation signal and as a membrane anchor. The family members also contain three additional transmembrane regions, at least seven conserved cysteines residues, and are of approximately the same size (218 to 284 residues). These proteins are collectively know as the “transmembrane 4 superfamily” (TM4) because they span plasma membrane four times. A schematic diagram of the domain structure of these proteins is as follows:

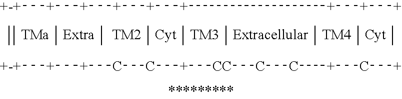

where Cyt is the cytoplasmic domain, TMa is the transmembrane anchor; TM2 to TM4 represents transmembrane regions 2 to 4, ‘C’ are conserved cysteines, and ‘*’ indicates the position of the consensus pattern. The consensus pattern spans a conserved region including two cysteines located in a short cytoplasmic loop between two transmembrane domains: Consensus pattern: G-x(3)-[LIVMF]-x(2)-[GSA]-[LIVMF](2)-G-C-x-[GA]-[STA]-x(2)-[EG]-x(2)-[CWN]-[LIVM](2).

b) Seven Transmembrane Integral Membrane Proteins.

SEQ ID NOS: 24, 41, 101, 157, 291, 305, 315, and 341 correspond to a sequence encoding a polypeptide that is a member of the seven transmembrane receptor family. G-protein coupled receptors (Strosberg, Eur. J. Biochem. (1991)196:1; Kerlavage, Curr. Opin. Struct. Biol. (1991) 1:394; and Probst et al., DNA Cell Biol. (1992) 11:1; and Savarese et al., Biochem. J. (1992) 293:1) (also called R7G) are an extensive group of hormones, neurotransmitters, odorants and light receptors which transduce extracellular signals by interaction with guanine nucleotide-binding (G) proteins. The tertiary structure of these receptors is thought to be highly similar. They have seven hydrophobic regions, each of which most probably spans the membrane. The N-terminus is located on the extracellular side of the membrane and is often glycosylated, while the C-terminus is cytoplasmic and generally phosphorylated. Three extracellular loops alternate with three intracellular loops to link the seven transmembrane regions. Most, but not all of these receptors, lack a signal peptide. The most conserved parts of these proteins are the transmembrane regions and the first two cytoplasmic loops. A conserved acidic-Arg-aromatic triplet is present in the N-terminal extremity of the second cytoplasmic loop (Attwood et al., Gene (1991) 98:153) and could be implicated in the interaction with G proteins.

To detect this widespread family of proteins a pattern is used that contains the conserved triplet and that also spans the major part of the third transmembrane helix. Additional information about the seven transmembrane receptor family, and methods for their identification and use, is found in U.S. Pat. No. 5,759,804. Due in part to their expression on the cell surface and other attractive characteristics, seven transmembrane protein family members are of particular interest as drug targets, as surface antigen markers, and as drug delivery targets (e.g., using antibody-drug complexes and/or use of anti-seven transmembrane protein antibodies as therapeutics in their own right).

c) Ank Repeats.

SEQ ID NOS: 116 and 251 represent polynucleotides encoding Ank repeat-containing proteins. The ankyrin motif is a 33 amino acid sequence named after the protein ankyrin which has 24 tandem 33-amino-acid motifs. Ank repeats were originally identified in the cell-cycle-control protein cdc10 (Breeden et al., Nature (1987) 329:651). Proteins containing ankyrin repeats include ankyrin, myotropin, 1-kappaB proteins, cell cycle protein cdc10, the Notch receptor (Matsuno et al., Development (1997) 124(21):4265); G9a (or BAT8) of the class III region of the major histocompatibility complex (Biochem J. 290:811-818, 1993), FABP, GABP, 53BP2, Lin12, glp-1, SW14, and SW16. The functions of the ankyrin repeats are compatible with a role in protein-protein interactions (Bork, Proteins (1993) 17(4):363; Lambert and Bennet, Eur. J. Biochem. (1993) 211:1; Kerr et al., Current Op. Cell Biol. (1992) 4:496; Bennet et al., J. Biol. Chem. (1980) 255:6424).

The 90 kD N-terminal domain of ankyrin contains a series of 24 33-amino-acid ank repeats. (Lux et al., Nature (1990) 344:36-42, Lambert et al., PNAS USA (1990) 87:1730.) The 24 ank repeats form four folded subdomains of 6 repeats each. These four repeat subdomains mediate interactions with at least 7 different families of membrane proteins. Ankyrin contains two separate binding sites for anion exchanger dimers. One site utilizes repeat subdomain two (repeats 7-12) and the other requires both repeat subdomains 3 and 4 (repeats 13-24). Since the anion exchangers exist in dimers, ankyrin binds 4 anion exchangers at the same time. (Michaely and Bennett, J. Biol. Chem. (1995) 270(37):22050) The repeat motifs are involved in ankyrin interaction with tubulin, spectrin, and other membrane proteins. (Lux et al., Nature (1990) 344:36.)

The Rel/NF-kappaB/Dorsal family of transcription factors have activity that is controlled by sequestration in the cytoplasm in association with inhibitory proteins referred to as I-kappaB. (Gilmore, Cell (1990) 62:841; Nolan and Baltimore, Curr Opin Genet Dev. (1992) 2:211; Baeuerle, Biochim Biophys Acta (1991) 1072:63; Schmitz et al., Trends Cell Biol. (1991) 1:130.) I-kappaB proteins contain 5 to 8 copies of 33 amino acid ankyrin repeats and certain NF-kappaB/rel proteins are also regulated by cis-acting ankyrin repeat containing domains including p105NF-kappaB which contains a series of ankyrin repeats (Diehl and Hannink, J. Virol. (1993) 67(12):7161). The I-kappaBs and Cactus (also containing ankyrin repeats) inhibit activators through differential interactions with the Rel-homology domain. The gene family includes proto-oncogenes, thus broadly implicating I-kappaB in the control of both normal gene expression and the aberrant gene expression that makes cells cancerous. (Nolan and Baltimore, Curr Opin Genet Dev. (1992) 2(2):211-220). In the case of rel/NF-kappaB and pp40/I-kappaBβ, both the ankyrin repeats and the carboxy-terminal domain are required for inhibiting DNA-binding activity and direct association of pp40/I-kappaBβ with rel/NF-kappaB protein. The ankyrin repeats and the carboxy-terminal of pp40/I-kappaBβ (form a structure that associates with the rel homology domain to inhibit DNA binding activity (Inoue et al., PNAS USA (1992) 89:4333).

The 4 ankyrin repeats in the amino terminus of the transcription factor subunit GABPβ are required for its interaction with the GABPα subunit to form a functional high affinity DNA-binding protein. These repeats can be crosslinked to DNA when GABP is bound to its target sequence. (Thompson et al., Science (1991) 253:762; LaMarco et al., Science (1991) 253:789).

Myotrophin, a 12.5 kDa protein having a key role in the initiation of cardiac hypertrophy, comprises ankyrin repeats. The ankyrin repeats are characteristic of a hairpin-like protruding tip followed by a helix-turn-helix motif. The V-shaped helix-turn-helix of the repeats stack sequentially in bundles and are stabilized by compact hydrophobic cores, whereas the protruding tips are less ordered.

d) ATPases Associated with Various Cellular Activities (AAA).

SEQ ID NOS: 63, 116, 134, 136, 151, 384, and 404 polynucleotides encoding novel members of the “ATPases Associated with diverse cellular Activities” (AAA) protein family The AAA protein family is composed of a large number of ATPases that share a conserved region of about 220 amino acids that contains an ATP-binding site (Froehlich et al., J. Cell Biol. (1991) 114:443; Erdmann et al. Cell (1991) 64:499; Peters et al., EMBO J. (1990) 9:1757; Kunau et al., Biochimie (1993) 75:209-224; Confalonieri et al., BioEssays (1995) 17:639; http://yeamob.pci.chemie.uni-tuebingen.de/AAA/Description.html). The proteins that belong to this family either contain one or two AAA domains.

Proteins containing two AAA domains include: 1) Mammalian and drosophila NSF (N-ethylmaleimide-sensitive fusion protein) and the fungal homolog, SEC18, which are involved in intracellular transport between the endoplasmic reticulum and Golgi, as well as between different Golgi cisternae; 2) Mammalian transitional endoplasmic reticulum ATPase (previously known as p97 or VCP), which is involved in the transfer of membranes from the endoplasmic reticulum to the golgi apparatus. This ATPase forms a ring-shaped homooligomer composed of six subunits. The yeast homolog, CDC48, plays a role in spindle pole proliferation; 3) Yeast protein PAS1 essential for peroxisome assembly and the related protein PAS1 from Pichia pastoris; 4) Yeast protein AFG2; 5) Sulfolobus acidocaldarius protein SAV and Halobacterium salinarium cdcH, which may be part of a transduction pathway connecting light to cell division.

Proteins containing a single AAA domain include: 1) Escherichia coli and other bacteria ftsH (or hflB) protein. FtsH is an ATP-dependent zinc metallopeptidase that degrades the heat-shock sigma-32 factor, and is an integral membrane protein with a large cytoplasmic C-terminal domain that contain both the AAA and the protease domains; 2) Yeast protein YME1, a protein important for maintaining the integrity of the mitochondrial compartment. YME1 is also a zinc-dependent protease; 3) Yeast protein AFG3 (or YTA10). This protein also contains an AAA domain followed by a zinc-dependent protease domain; 4) Subunits from regulatory complex of the 26S proteasome (Hilt et al., Trends Biochem. Sci. (1996) 21:96), which is involved in the ATP-dependent degradation of ubiquitinated proteins, which subunits include: a) Mammalian 4 and homologs in other higher eukaryotes, in yeast (gene YTA5) and fission yeast (gene mts2); b) Mammalian 6 (TBP7) and homologs in other higher eukaryotes and in yeast (gene YTA2); c) Mammalian subunit 7 (MSS1) and homologs in other higher eukaryotes and in yeast (gene CIM5 or YTA3); d) Mammalian subunit 8 (P45) and homologs in other higher eukaryotes and in yeast (SUG1or CIM3 or TBYI) and fission yeast (gene let1); e) Other probable subunits include human TBP1, which influences HIV gene expression by interacting with the virus tat transactivator protein, and yeast YTA1 and YTA6; 5) Yeast protein BCS1, a mitochondrial protein essential for the expression of the Rieske iron-sulfur protein; 6) Yeast protein MSP1, a protein involved in intramitochondrial sorting of proteins; 7) Yeast protein PAS8, and the corresponding proteins PAS5 from Pichia pastoris and PAY4 from Yarrowia lipolytica; 8) Mouse protein SKD1 and its fission yeast homolog (SpAC2G11.06); 9) Caenorhabditis elegans meiotic spindle formation protein mei-1; 10) Yeast protein SAP1′ 11) Yeast protein YTA7; and 12) Mycobacterium leprae hypothetical protein A2126A.

In general, the AAA domains in these proteins act as ATP-dependent protein clamps(Confalonieri et al. (1995) BioEssays 17:639). In addition to the ATP-binding ‘A’ and ‘B’ motifs, which are located in the N-terminal half of this domain, there is a highly conserved region located in the central part of the domain which was used in the development of the signature pattern. The consensus pattern is: [LIVMT]-x-[LIVMT]-[LIVMF]-x-[GATMC]-[ST]-[NS]-x(4)-[LIVM]-D-x-A-[LIFA]-x-R.

e) Basic Region Plus Leucine Zipper Transcription Factors.

SEQ ID NO:374 correspond to a polynucleotide encoding a novel member of the family of basic region plus leucine zipper transcription factors. The bZIP superfamily (Hurst, Protein Prof. (1995) 2:105; and Ellenberger, Curr. Opin. Struct. Biol. (1994) 4:12) of eukaryotic DNA-binding transcription factors encompasses proteins that contain a basic region mediating sequence-specific DNA-binding followed by a leucine zipper required for dimerization. Members of the family include transcription factor AP-1, which binds selectively to enhancer elements in the cis control regions of SV40 and metallothionein IIA. AP-1, also known as c-jun, is the cellular homolog of the avian sarcoma virus 17 (ASV 17) oncogene v-jun.

Other members of this protein family include jun-B and jun-D, probable transcription factors that are highly similar to jun/AP-1; the fos protein, a proto-oncogene that forms a non-covalent dimer with c-jun; the fos-related proteins fra-1, and fos B; and mammalian cAMP response element (CRE) binding proteins CREB, CREM, ATF-1, ATF-3, ATF-4, ATF-5, ATF-6 and LRF-1. The consensus pattern for this protein family is: [KR]-x(1,3)-[RKSAQ]-N-x(2)-[SAQ](2)-x-[RKTAENQ]-x-R-x-[RK].

f) Bromodomain.

SEQ ID NO:97 corresponds to a polynucleotide encoding a polypeptide having a bromodomain region (Haynes et al., 1992, Nucleic Acids Res. 20:2693-2603, Tamnkun et al., 1992, Cell 68:561-572, and Tamkun, 1995, Curr. Opin. Genet. Dev. 5:473-477), which is a conserved region of about 70 amino acids found in the following proteins: 1) Higher eukaryotes transcription initiation factor TFIID 250 Kd subunit (TBP-associated factor p250) (gene CCG1); P250 is associated with the TFIID TATA-box binding protein and seems essential for progression of the GI phase of the cell cycle. 2) Human RING3, a protein of unknown function encoded in the MHC class II locus; 3) Mammalian CREB-binding protein (CBP), which mediates cAMP-gene regulation by binding specifically to phosphorylated CREB protein; 4) Mammalian homologs of brahma, including three brahma-like human: SNF2a(hBRM), SNF2b, and BRG1; 5) Human BS69, a protein that binds to adenovirus E1A and inhibits E1A transactivation; 6) Human peregrin (or Br140).

The bromodomain is thought to be involved in protein-protein interactions and may be important for the assembly or activity of multicomponent complexes involved in transcriptional activation. The consensus pattern, which spans a major part of the bromodomain, is: [STANVF]-x(2)-F-x(4)-[DNS]-x(5,7)-[DENQTF]-Y-[HFY]-x(2)-[LIVMFY]-x(3)-[LIVM]-x(4)-[LIVM]-x(6,8)-Y-x(12,13)-[LIVM]-x(2)-N-[SACF]-x(2)-[FY].

g) EF-Hand.

SEQ ID NOS:136, 242, and 379 correspond to polynucleotides encoding a novel protein in the family of EF-hand proteins. Many calcium-binding proteins belong to the same evolutionary family and share a type of calcium-binding domain known as the EF-hand (Kawasaki et al., Protein. Prof. (1995) 2:305-490). This type of domain consists of a twelve residue loop flanked on both sides by a twelve residue alpha-helical domain. In an EF-hand loop the calcium ion is coordinated in a pentagonal bipyramidal configuration. The six residues involved in the binding are in positions 1, 3, 5, 7, 9 and 12; these residues are denoted by X, Y, Z, -Y, -X and -Z. The invariant Glu or Asp at position 12 provides two oxygens for liganding Ca (bidentate ligand).

Proteins known to contain EF-hand regions include: Calmodulin (Ca=4, except in yeast where Ca=3) (“Ca=” indicates approximate number of EF-hand regions); diacylglycerol kinase (EC 2.7.1.107) (DGK) (Ca=2); 2) FAD-dependent glycerol-3-phosphate dehydrogenase (EC 1.1.99.5) from mammals (Ca=1); guanylate cyclase activating protein (GCAP) (Ca=3); MIF related proteins 8 (MRP-8 or CFAG) and 14 (MRP-14) (Ca=2); myosin regulatory light chains (Ca=1); oncomodulin (Ca=2); osteonectin (basement membrane protein BM-40) (SPARC); and proteins that contain an “osteonectin” domain (QR1, matrix glycoprotein SC1).

The consensus pattern includes the complete EF-hand loop as well as the first residue which follows the loop and which seem to always be hydrophobic.

Consensus pattern: D-x-[DNS]-{ILVFYW}-[DENSTG]-[DNQGHRK]-{GP}-[LIVMC]-[DENQSTAGC]-x(2)-[DE]-[LIVMFYW]

h) Eukaryotic Aspartyl Proteases.

SEQ ID NO:308 corresponds to a gene encoding a novel eukaryotic aspartyl protease. Aspartyl proteases, known as acid proteases, (EC 3.4.23.-) are a widely distributed family of proteolytic enzymes (Foltmann B., Essays Biochem. (1981) 17:52; Davies D. R., Annu. Rev. Biophys. Chem. (1990) 19:189; Rao J. K. M., et al., Biochemistry (1991) 30:4663) known to exist in vertebrates, fungi, plants, retroviruses and some plant viruses. Aspartate proteases of eukaryotes are monomeric enzymes which consist of two domains. Each domain contains an active site centered on a catalytic aspartyl residue. The two domains most probably evolved from the duplication of an ancestral gene encoding a primordial domain. Currently known eukaryotic aspartyl proteases include: 1) Vertebrate gastric pepsins A and C (also known as gastricsin); 2) Vertebrate chymosin (rennin), involved in digestion and used for making cheese; 3) Vertebrate lysosomal cathepsins D (EC 3.4.23.5) and E (EC 3.4.23.34); 4) Mammalian renin (EC 3.4.23.15) whose function is to generate angiotensin I from angiotensinogen in the plasma; 5) Fungal proteases such as aspergillopepsin A (EC 3.4.23.18), candidapepsin (EC 3.4.23.24), mucoropepsin (EC 3.4.23.23) (mucor rennin), endothiapepsin (EC 3.4.23.22), polyporopepsin (EC 3.4.23.29), and rhizopuspepsin (EC 3.4.23.21); and 6) Yeast saccharopepsin (EC 3.4.23.25) (proteinase A) (gene PEP4). PEP4 is implicated in posttranslational regulation of vacuolar hydrolases; 7) Yeast barrierpepsin (EC 3.4.23.35) (gene BAR 1); a protease that cleaves alpha-factor and thus acts as an antagonist of the mating pheromone; and 8) Fission yeast sxal which is involved in degrading or processing the mating pheromones.

Most retroviruses and some plant viruses, such as badnaviruses, encode for an aspartyl protease which is an homodimer of a chain of about 95 to 125 amino acids. In most retroviruses, the protease is encoded as a segment of a polyprotein which is cleaved during the maturation process of the virus. It is generally part of the pol polyprotein and, more rarely, of the gag polyprotein. Because the sequence around the two aspartates of eukaryotic aspartyl proteases and around the single active site of the viral proteases is conserved, a single signature pattern can be used to identify members of both groups of proteases. The consensus pattern is: [LIVMFGAC]-[LIVMTADN]-[LIVFSA]-D-[ST]-G-[STAV]-[STAPDENQ]-x-[LIVMFSTNC]-x-[LIVMFGTA], where D is the active site residue.

i) GATA Family of Transcription Factors.

SEQ ID NO:213 corresponds to a novel member of the GATA family of transcription factors. The GATA family of transcription factors are proteins that bind to DNA sites with the consensus sequence (A/T)GATA(A/G), found within the regulatory region of a number of genes. Proteins currently known to belong to this family are: 1) GATA-1 (Trainor, C. D., et al., Nature (1990) 343:92) (also known as Eryf1, GF-1 or NF-E1), which binds to the GATA region of globin genes and other genes expressed in erythroid cells. It is a transcriptional activator which probably serves as a general ‘switch’ factor for erythroid development; 2) GATA-2 (Lee, M. E., et al., J. Biol. Chem. (1991) 266:16188), a transcriptional activator which regulates endothelin-1 gene expression in endothelial cells; 3) GATA-3 (Ho, I. -C., et al., EMBO J. (1991) 10:1187), a transcriptional activator which binds to the enhancer of the T-cell receptor alpha and delta genes; 4) GATA-4 (Spieth, J., et al., Mol. Cell. Biol. (1991) 11:4651), a transcriptional activator expressed in endodermally derived tissues and heart; 5) Drosophila protein pannier (or DGATAa) (gene pnr) which acts as a repressor of the achaete-scute complex (as-c); 6) Bombyx mori BCFI (Drevet, J. R., et al., J Biol. Chem. (1994) 269:10660), which regulates the expression of chorion genes; 7) Caenorhabditis elegans elt-1 and elt-2, transcriptional activators of genes containing the GATA region, including vitellogenin genes (Hawkins, M. G., et al., J. Biol. Chem. (1995) 270:14666); 8) Ustilago maydis urbs1 (Voisard, C. P. O., et al., Mol. Cell. Biol. (1993) 13:7091), a protein involved in the repression of the biosynthesis of siderophores; 9) Fission yeast protein GAF2.

All these transcription factors contain a pair of highly similar ‘zinc finger’ type domains with the consensus sequence C-x2-C-x17-C-x2-C. Some other proteins contain a single zinc finger motif highly related to those of the GATA transcription factors. These proteins are: 1) Drosophila box A-binding factor (ABF) (also known as protein serpent (gene srp)) which may function as a transcriptional activator protein and may play a key role in the organogenesis of the fat body; 2) Emericella nidulans are (Arst, H. N., Jr., et al., Trends Genet. (1989) 5:291) a transcriptional activator which mediates nitrogen metabolite repression; 3) Neurospora crassa nit-2 (Fu, Y. -H., et al., Mol. Cell. Biol. (1990) 10:1056), a transcriptional activator which turns on the expression of genes coding for enzymes required for the use of a variety of secondary nitrogen sources, during conditions of nitrogen limitation; 4) Neurospora crassa white collar proteins 1 and 2 (WC-1 and WC-2), which control expression of light-regulated genes; 5) Saccharomyces cerevisiae DAL81 (or UGA43), a negative nitrogen regulatory protein; 6) Saccharomyces cerevisiae GLN3, a positive nitrogen regulatory protein; 7) Saccharomyces cerevisiae GAT1; 8) Saccharomyces cerevisiae GZF3.

The consensus pattern for the GATA family is: C-x-[DN]-C-x(4,5)-[ST]-x(2)-W-[HR]-[RK]-x(3)-[GN]-x(3,4)-C-N-[AS]-C, where the four C's are zinc ligands.

j) G-Protein Alpha Subunit.

SEQ ID NO:367 corresponds to a gene encoding a novel polypeptide of the G-protein alpha subunit family. Guanine nucleotide binding proteins (G-proteins) are a family of membrane-associated proteins that couple extracellularly-activated integral-membrane receptors to intracellular effectors, such as ion channels and enzymes that vary the concentration of second messenger molecules. G-proteins are composed of 3 subunits (alpha, beta and gamma) which, in the resting state, associate as a trimer at the inner face of the plasma membrane. The alpha subunit has a molecule of guanosine diphosphate (GDP) bound to it. Stimulation of the G-protein by an activated receptor leads to its exchange for GTP (guanosine triphosphate). This results in the separation of the alpha from the beta and gamma subunits, which always remain tightly associated as a dimer. Both the alpha and beta-gamma subunits are then able to interact with effectors, either individually or in a cooperative manner. The intrinsic GTPase activity of the alpha subunit hydrolyses the bound GTP to GDP. This returns the alpha subunit to its inactive conformation and allows it to reassociate with the beta-gamma subunit, thus restoring the system to its resting state.

G-protein alpha subunits are 350-400 amino acids in length and have molecular weights in the range 40-45 kDa. Seventeen distinct types of alpha subunit have been identified in mammals. These fall into 4 main groups on the basis of both sequence similarity and function: alpha-s, alpha-q, alpha-i and alpha-12 (Simon et al., Science (1993) 252:802). Many alpha subunits are substrates for ADP-ribosylation by cholera or pertussis toxins. They are often N-terminally acylated, usually with myristate and/or palmitoylate, and these fatty acid modifications are probably important for membrane association and high-affinity interactions with other proteins. The atomic structure of the alpha subunit of the G-protein involved in mammalian vision, transducin, has been elucidated in both GTP- and GDB-bound forms, and shows considerable similarity in both primary and tertiary structure in the nucleotide-binding regions to other guanine nucleotide binding proteins, such as p21-ras and EF-Tu.

k) Phorbol Esters/Diacylglycerol Binding.

SEQ ID NO:188 and 251 represent polynucleotides encoding a protein belonging to the family including phorbol esters/diacylglycerol binding proteins. Diacylglycerol (DAG) is an important second messenger. Phorbol esters (PE) are analogues of DAG and potent tumor promoters that cause a variety of physiological changes when administered to both cells and tissues. DAG activates a family of serine/threonine protein kinases, collectively known as protein kinase C (PKC) (Azzi et al., Eur. J. Biochem. (1992) 208:547). Phorbol esters can directly stimulate PKC. The N-terminal region of PKC, known as C1, has been shown (Ono et al., Proc. Natl. Acad. Sci. USA (1989) 86:4868) to bind PE and DAG in a phospholipid and zinc-dependent fashion. The C1 region contains one or two copies (depending on the isozyme of PKC) of a cysteine-rich domain about 50 amino-acid residues long and essential for DAG/PE-binding. Such a domain has also been found in, for example, the following proteins.

(1) Diacylglycerol kinase (EC 2.7.1.107) (DGK) (Sakane et al., Nature (1990) 344:345), the enzyme that converts DAG into phosphatidate. It contains two copies of the DAG/PE-binding domain in its N-terminal section. At least five different forms of DGK are known in mammals; and

(2) N-chimaerin, a brain specific protein which shows sequence similarities with the BCR protein at its C-terminal part and contains a single copy of the DAG/PE-binding domain at its N-terminal part. It has been shown (Ahmed et al., Biochem. J. (1 990) 2 72:767, and Ahmed et al., Biochem. J. (1 991) 280:23 3) to be able to bind phorbol esters.

The DAG/PE-binding domain binds two zinc ions; the ligands of these metal ions are probably the six cysteines and two histidines that are conserved in this domain. The signature pattern completely spans the DAG/PE domain. The consensus pattern is: H-x-[LIVMFYW]-x(8, 11)-C-x(2)-C-x(3)-[LIVMFC]-x(5,10)-C-x(2)-C-x(4)-[HD]-x(2)-C-x(5,9)-C. All the C and H are probably involved in binding zinc.

1) Protein Kinase.

SEQ ID NOS:202, 315, 367, and 397 represent polynucleotides encoding protein kinases. Protein kinases catalyze phosphorylation of proteins in a variety of pathways, and are implicated in cancer. Eukaryotic protein kinases (Hanks S. K., et al., FASEB J. (1995) 9:576; Hunter T., Meth. Enzymol.(1991)200:3; Hanks S. K., et al., Meth. Enzymol. (1991) 200:38; Hanks S. K., Curr. Opin. Struct. Biol. (1991) 1:369; Hanks S. K., et al., Science (1988) 241:42) are enzymes that belong to a very extensive family of proteins which share a conserved catalytic core commnon to both serine/threonine and tyrosine protein kinases. There are a number of conserved regions in the catalytic domain of protein kinases. Two of the conserved regions are the basis for the signature pattern in the protein kinase profile. The first region, which is located in the N-terminal extremity of the catalytic domain, is a glycine-rich stretch of residues in the vicinity of a lysine residue, which has been shown to be involved in ATP binding. The second region, which is located in the central part of the catalytic domain, contains a conserved aspartic acid residue which is important for the catalytic activity of the enzyme (Knighton D. R., et al., Science (1991) 253:407). The protein kinase profile includes two signature patterns for this second region: one specific for serine/threonine kinases and the other for tyro sine kinases. A third profile is based on the alignment in (Hanks S. K., et al., FASEB J. (1995) 9:576) and covers the entire catalytic domain. The consensus patterns are as follows:

1) Consensus pattern: [LIV]-G-{P}-G-{P}-[FYWMGSTNH]-[SGA]-{PW}-[LIVCAT]-{PDI}-x-[GSTACLIVMFY]-x(5,18)-[LIVMFYWCSTAR]-[AIVP]-[LIVMFAGCKR]-K, where K binds ATP. The majority of known protein ki-nases are detected by this pattern. Proteins kinases that are not detected by this consensus include viral kinases, which are quite divergent in this region and are completely missed by this pattern.

2) Consensus pattern: [LIVMFYC]-x-[HY]-x-D-[LIVMFY]-K-x(2)-N-[LIVMFYCT](3), where D is an active site residue. This consensus sequence identifies most serine/threonine-specific protein kinases with only 10 exceptions. Half of the exceptions are viral kinases, while the other exceptions include Epstein-Barr virus BGLF4 and Drosophila ninaC, which have Ser and Arg, respectively, instead of the conserved Lys. These latter two protein kinases are detected by the tyrosine kinase specific pattern described below.

3) Consensus pattern: [LIVMFYC]-x-[HY]-x-D-[LIVMFY]-[RSTAC]-x(2)-N-[LIVMFYC], where D is an active site residue. All tyrosine-specific protein kinases are detected by this consensus pattern, with the exception of human ERBB3 and mouse blk. This pattern also detects most bacterial aminoglycoside phosphotransferases (Benner S., Nature (1987) 329:21; Kirby R., J. Mol. Evol. (1992) 30:489) and herpesviruses ganciclovir kinases (Littler E., et al., Nature (1992) 358:160), which are structurally and evolutionary related to protein kinases.

The protein kinase profile also detects receptor guanylate cyclases and 2-5A-dependent ribonucleases. Sequence similarities between these two families and the eukaryotic protein kinase family have been noticed previously. The profile also detects Arabidopsis thaliana kinase-like protein TMKL1 which seems to have lost its catalytic activity.

If a protein analyzed includes the two of the above protein kinase signatures, the probability of it being a protein kinase is close to 100%. Eukaryotic-type protein kinases have also been found in prokaryotes such as Myxococcus xanthus (Munoz-Dorado J., et al., Cell (1991) 67:995) and Yersinia pseudotuberculosis. The patterns shown above has been updated since their publication in (Bairoch A., et al., Nature (1988) 331:22).

m) Protein Phosphatase 2C, SEQ ID NO:256 corresponds to a polynucleotide encoding a novel protein phosphatase 2C (PP2C), which is one of the four major classes of mammalian serine/threonine specific protein phosphatases. PP2C (Wenk et al., FEBS Lett. (1992) 297:135) is a monomeric enzyme of about 42 Kd which shows broad substrate specificity and is dependent on divalent cations (mainly manganese and magnesium) for its activity. Three isozymes are currently known in mammals: PP2C-alpha, -beta and -gamma.

n) Protein Tyrosine Phosphatase.

SEQ ID NO:382 represents a polynucleotide encoding a protein tyrosine kinase. Tyrosine specific protein phosphatases (EC 3.1.3.48) (PTPase) (Fischer et al., Science (1991) 253:401; Charbonneau et al., Annu. Rev. Cell Biol. (1992) 8:463; Trowbridge, J. Biol Chem. (1991) 266:23517; Tonks et al., Trends Biochem. Sci. (1989) 14:497; and Hunter, Cell (1989) 58:1013) catalyze the removal of a phosphate group attached to a tyrosine residue. These enzymes are very important in the control of cell growth, proliferation, differentiation and transformation. Multiple forms of PTPase have been characterized and can be classified into two categories: soluble PTPases and transmembrane receptor proteins that contain PTPase domain(s).

Soluble PTPases include PTPN3 (H1) and PTPN4 (MEG), enzymes that contain an N-terminal band 4.1-like domain and could act at junctions between the membrane and cytoskeleton; PTPN6 (PTP-1C; HCP; SHP) and PTPN11(PTP-2C; SH-PTP3; Syp), enzymes that contain two copies of the SH2 domain at its N-terminal extremity.

Dual specificity PTPases include DUSP1 (PTPN10; MAP kinase phosphatase-1; MKP-1) which dephosphorylates MAP kinase on both Thr-183 and Tyr-185; and DUSP2 (PAC-1), a nuclear enzyme that dephosphorylates MAP kinases ERK1 and ERK2 on both Thr and Tyr residues.

Structurally, all known receptor PTPases are made up of a variable length extracellular domain, followed by a transmembrane region and a C-terminal catalytic cytoplasmic domain. Some of the receptor PTPases contain fibronectin type III (FN-III) repeats, immunoglobulin-like domains, MAM domains or carbonic anhydrase-like domains in their extracellular region. The cytoplasmic region generally contains two copies of the PTPAse domain. The first seems to have enzymatic activity, while the second is inactive but seems to affect substrate specificity of the first. In these domains, the catalytic cysteine is generally conserved but some other, presumably important, residues are not.

PTPase domains consist of about 300 amino acids. There are two conserved cysteines and the second one has been shown to be absolutely required for activity. Furthermore, a number of conserved residues in its immediate vicinity have also been shown to be important. The consensus pattern for PTPases is: [LIVMF]-H-C-x(2)-G-x(3)-[STC]-[STAGP]-x-[LIVMFY]; C is the active site residue.

o) SH3 Domain.

SEQ ID NO:306 and 386 represent polynucleotides encoding SH3 domain proteins. The Src homology 3 (SH3) domain is a small protein domain of about 60 amino acid residues first identified as a conserved sequence in the non-catalytic part of several cytoplasmic protein tyrosine kinases (e.g. Src, Abl, Lck) (Mayer et al., Nature (1988) 332:272). The domain has also been found in a variety of intracellular or membrane-associated proteins (Musacchio et al., FEBS Lett. (1992) 307:55; Pawson et al., Curr. Biol. (1993) 3:434; Mayer et al., Trends Cell Biol. (1993) 3:8; and Pawson et al., Nature (1995) 373:573).

The SH3 domain has a characteristic fold that consists of five or six beta-strands arranged as two tightly packed anti-parallel beta sheets. The linker regions may contain short helices (Kuriyan et al., Curr. Opin. Struct. Biol. (1993) 3:828). It is believed that SH3 domain-containing proteins mediate assembly of specific protein complexes via binding to proline-rich peptides (Morton et al., Curr. Biol. (1994) 4:615). In general, SH3 domains are found as single copies in a given protein, but there is a significant number of proteins with two SH3 domains and a few with 3 or 4 copies.

SH3 domains have been identified in, for example, protein tyrosine kinases, such as the Src, Abl, Bkt, Csk and ZAP70 families of kinases; mammalian phosphatidylinositol-specific phospholipase C-gamma-1 and -2; mammalian phosphatidyl inositol 3-kinase regulatory p85 subunit; mammalian Ras GTPase-activating protein (GAP); mammalian Vav oncoprotein, a guanine nucleotide exchange factor of the CDC24 family; Drosophila lethal(1)discs large-1 tumor suppressor protein (gene Dlg1); mammalian tight junction protein ZO-1; vertebrate erythrocyte membrane protein p55; Caenorhabditis elegans protein lin-2; rat protein CASK; and mammalian synaptic proteins SAP90/PSD-95, CHAPSYN-110/PSD-93, SAP97/DLG1 and SAP102. Novel SH3-domain containing polypeptides will facilitate elucidation of the role of such proteins in important biological pathways, such as ras activation.

p) Trypsin.

SEQ ID NO:169 corresponds to a novel serine protease of the trypsin family. The catalytic activity of the serine proteases from the trypsin family is provided by a charge relay system involving an aspartic acid residue hydrogen-bonded to a histidine, which itself is hydrogen-bonded to a serine. The sequences in the vicinity of the active site serine and histidine residues are well conserved in this family of proteases (Brenner S., Nature (1988) 334:528). Proteases known to belong to the trypsin family include: 1) Acrosin; 2) Blood coagulation factors VII, IX, X, XI and XII, thrombin, plasminogen, and protein C; 3) Cathepsin G; 4) Chymotrypsins; 5) Complement components C1r, C1s, C2, and complement factors B, D and I; 6) Complement-activating component of RA-reactive factor; 7) Cytotoxic cell proteases (granzymes A to H); 8) Duodenase I; 9) Elastases 1, 2, 3A, 3B (protease E), leukocyte (medullasin).; 10) Enterokinase (EC 3.4.21.9) (enteropeptidase); 11) Hepatocyte growth factor activator; 12) Hepsin; 13) Glandular (tissue) kallikreins (including EGF-binding protein types A, B, and C, NGF-gamma chain, gamma-renin, prostate specific antigen (PSA) and tonin); 14) Plasma kallikrein; 15) Mast cell proteases (MCP) 1 (chymase) to 8; 16) Myeloblastin (proteinase 3) (Wegener's autoantigen); 17) Plasminogen activators (urokinase-type, and tissue-type); 18) Trypsins I, II, III, and IV; 19) Tryptases; 20) Snake venom proteases such as ancrod, batroxobin, cerastobin, flavoxobin, and protein C activator; 21) Collagenase from common cattle grub and collagenolytic protease from Atlantic sand fiddler crab; 22) Apolipoprotein(a); 23) Blood fluke cercarial protease; 24) Drosophila trypsin like proteases: alpha, easter, snake-locus; 25) Drosophila protease stubble (gene sb); and 26) Major mite fecal allergen Der p III. All the above proteins belong to family S1 in the classification of peptidases (Rawlings N. D., et al., Meth. Enzymol. (1994) 244:19; http://www.expasy.ch/cgi-bin/lists?peptidas.txt) and originate from eukaryotic species. It should be noted that bacterial proteases that belong to family S2A are similar enough in the regions of the active site residues that they can be picked up by the same patterns.

The consensus patterns for this trypsin protein family are: 1) [LIVM]-[ST]-A-[STAG]-H-C, where H is the active site residue. All sequences known to belong to this class detected by the pattern, except for complement components C1r and C1s, pig plasminogen, bovine protein C, rodent urokinase, ancrod, gyroxin and two insect trypsins; 2) [DNSTAGC]-[GSTAPIMVQH]-x(2)-G-[DE]-S-G-[GS]-[SAPHV]-[LIVMFYWH]-[LIVMFYSTANQH], where S is the active site residue. All sequences known to belong to this family are detected by the above consensus sequences, except for 18 different proteases which have lost the first conserved glycine. If a protein includes both the serine and the histidine active site signatures, the probability of it being a trypsin family serine protease is 100%.

q) WD Domain, G-Beta Repeats.

SEQ ID NOS:188 and 335 represent novel members of the WD domain/G-beta repeat family. Beta-transducin (G-beta) is one of the three subunits (alpha, beta, and gamma) of the guanine nucleotide-binding proteins (G proteins) which act as intermediaries in the transduction of signals generated by transmembrane receptors (Gilman, Annu. Rev. Biochem. (1987) 56:615). The alpha subunit binds to and hydrolyzes GTP; the functions of the beta and gamma subunits are less clear but they seem to be required for the replacement of GDP by GTP as well as for membrane anchoring and receptor recognition.

In higher eukaryotes, G-beta exists as a small multigene family of highly conserved proteins of about 340 amino acid residues. Structurally, G-beta consists of eight tandem repeats of about 40 residues, each containing a central Trp-Asp motif (this type of repeat is sometimes called a WD-40 repeat). Such a repetitive segment has been shown to exist in a number of other proteins including: human LIS1, a neuronal protein involved in type-1 lissencephaly; and mammalian coatomer beta′ subunit (beta′-COP), a component of a cytosolic protein complex that reversibly associates with Golgi membranes to form vesicles that mediate biosynthetic protein transport.

The consensus pattern for the WD domain/G-Beta repeat family is: [LIVMSTAC]-[LIVMFYWSTAGC]-[LIMSTAG]-[LIVMSTAGC]-x(2)-[DN]-x(2)-[LIVMWSTAC]-x-[LIVMFSTAG]-W-[DEN]-[LIVMFSTAGCN].

r) wnt Family of Developmental Signaling Proteins.

SEQ ID NO: 23, 291, 324, 330, 341, and 353 correspond to novel members of the wnt family of developmental signaling proteins. Wnt-1 (previously known as int-1), the seminal member of this family, (Nusse R., Trends Genet. (1988) 4:291) is a proto-oncogene induced by the integration of the mouse mammary tumor virus. It is thought to play a role in intercellular communication and seems to be a signalling molecule important in the development of the central nervous system (CNS). The sequence of wnt-1 is highly conserved in mammals, fish, and amphibians. Wnt-1 was found to be a member of a large family of related proteins (Nusse R., et al., Cell (1992) 69:1073; McMahon A. P., Trends Genet. (1992) 8:1; Moon R. T., BioEssays (1993) 15:91) that are all thought to be developmental regulators. These proteins are known as wnt-2 (also known as irp), wnt-3, -3A, -4, -5A, -5B, -6, -7A, -7B, -8, -8B, -9 and -10. At least four members of this family are present in Drosophila; one of them, wingless (wg), is implicated in segmentation polarity. All these proteins share the following features characteristics of secretory proteins: a signal peptide, several potential N-glycosylation sites and 22 conserved cysteines that are probably involved in disulfide bonds. The Wnt proteins seem to adhere to the plasma membrane of the secreting cells and are therefore likely to signal over only few cell diameters. The consensus pattern, which is based upon a highly conserved region including three cysteines, is as follows: C-K-C-H-G-[LIVMT]-S-G-x-C. All sequences known to belong to this family are detected by the provided consensus pattern.

s) Ww/rsp5/WWP Domain-Containing Proteins.

SEQ ID NOS:188, 379, and 395 represent polynucleotides encoding a polypeptide in the family of WW/rsp5/WWP domain-containing proteins. The WW domain (Bork et al., Trends Biochem. Sci. (1994) 19:531; Andre et al., Biochem. Biophys. Res. Commun. (1994) 205:1201; Hofmann et al., FEBS Lett. (1995) 358:153; and Sudol et al., FEBS Lett. (1995) 369:67), also known as rsp5 or WWP), was originally discovered as a short conserved region in a number of unrelated proteins, among them dystrophin, the gene responsible for Duchenne muscular dystrophy. The domain, which spans about 35 residues, is repeated up to 4 times in some proteins. It has been shown (Chen et al., Proc. Natl. Acad. Sci. USA (1995) 92:7819) to bind proteins with particular proline-motifs, [AP]-P-P-[AP]-Y, and thus resembles somewhat SH3 domains. It appears to contain beta-strands grouped around four conserved aromatic positions, generally Trp. The name WW or WWP derives from the presence of these Trp as well as that of a conserved Pro. It is frequently associated with other domains typical for proteins in signal transduction processes.

Proteins containing the WW domain include:

1. Dystrophin, a multidomain cytoskeletal protein. Its longest alternatively spliced form consists of an N-terminal actin-binding domain, followed by 24 spectrin-like repeats, a cysteine-rich calcium-binding domain and a C-terminal globular domain. Dystrophins form tetramers and is thought to have multiple functions including involvement in membrane stability, transduction of contractile forces to the extracellular environment and organization of membrane specialization. Mutations in the dystrophin gene lead to muscular dystrophy of Duchenne or Becker type. Dystrophin contains one WW domain C-terminal of the spectrin-repeats.

2. Vertebrate YAP protein, which is a substrate of an unknown serine kinase. It binds to the SH3 domain of the Yes oncoprotein via a proline-rich region. This protein appears in alternatively spliced isoforms, containing either one or two WW domains.

3. IQGAP, which is a human GTPase activating protein acting on ras. It contains an N-terminal domain similar to fly muscle mp20 protein and a C-terminal ras GTPase activator domain.

For the sensitive detection of WW domains, the profile spans the whole homology region as well as a pattern. The consensus for this family is: W-x(9,11)-[VFY]-[FYW]-x(6,7)-[GSTNE]-[GSTQCR]-[FYW]-x(2)-P.

t) Zinc Finger, C2H2 Type.

SEQ ID NO:61, 306, and 386 correspond to polynucleotides encoding novel members of the of the C2H2 type zinc finger protein family. Zinc finger domains (Klug et al., Trends Biochem. Sci. (1987) 12:464; Evans et al., Cell (1988) 52:1; Payre et al., FEBS Lett. (1988) 234:245; Miller et al., EMBO J. (1985) 4:1609; and Berg, Proc. Natl. Acad. Sci. USA (1988) 85:99) are nucleic acid-binding protein structures first identified in the Xenopus transcription factor TFIIIA. These domains have since been found in numerous nucleic acid-binding proteins. A zinc finger domain is composed of 25 to 30 amino acid residues. Two cysteine or histidine residues are positioned at both extremities of the domain, which are involved in the tetrahedral coordination of a zinc atom. It has been proposed that such a domain interacts with about five nucleotides.

Many classes of zinc fingers are characterized according to the number and positions of the histidine and cysteine residues involved in the zinc atom coordination. In the first class to be characterized, called C2H2, the first pair of zinc coordinating residues are cysteines, while the second pair are histidines. A number of experimental reports have demonstrated the zinc-dependent DNA or RNA binding property of some members of this class.

Mammalian proteins having a C2H2 zipper include (number in parenthesis indicates number of zinc finger regions in the protein): basonuclin (6), BCL-6/LAZ-3 (6), erythroid krueppel-like transcription factor (3), transcription factors Sp1 (3), Sp2 (3), Sp3 (3) and Sp(4) 3, transcriptional repressor YY1 (4), Wilms' tumor protein (4), EGR1/Krox24 (3), EGR2/Krox20 (3), EGR3/Pilot (3), EGR4/AT133 (4), Evi-1 (10), GLI1 (5), GLI2 (4+), GLI3 (3+), HIV-EP1/ZNF40 (4), HIV-EP2 (2), KR1 (9+), KR2 (9), KR3 (15+), KR4 (14+), KR5 (11+), HF.12 (6+), REX-1 (4), ZfX (13), ZfY (13), Zfp-35 (18), ZNF7 (15), ZNF8 (7), ZNF35 (10), ZNF42/MZF-1 (13), ZNF43 (22), ZNF46/Kup (2), ZNF76 (7), ZNF91 (36), ZNF133 (3).

In addition to the conserved zinc ligand residues, it has been shown that a number of other positions are also important for the structural integrity of the C2H2 zinc fingers. (Rosenfeld et al., J. Biomol. Struct. Dyn. (1993) 11:557) The best conserved position is found four residues after the second cysteine; it is generally an aromatic or aliphatic residue. The consensus pattern for C2H2 zinc fingers is: C-x(2,4)-C-x(3)-[LIVMFYWC]-x(8)-H-x(3,5)-H. The two C's and two H's are zinc ligands.

u) Zinc Finger, CCHC Class.

SEQ ID NO:322 corresponds to a polynucleotide encoding a novel member of the zinc finger CCHC family. The CCHC zinc finger protein family to date has been mostly composed of retroviral gag proteins (nucleocapsid). The prototype structure of this family is from HIV. The family also contains members involved in eukaryotic gene regulation, such as C. elegans GLH-1. The consensus sequence of this family is based upon the common structure of an 18-residue zinc finger.

v) Zinc-Binding Metalloprotease Domain.

SEQ ID NO:306 and 395 represent polynucleotides encoding novel members of the zinc-binding metalloprotease domain protein family. The majority of zinc-dependent metallopeptidases (with the notable exception of the carboxypeptidases) share a common pattern of primary structure (Jongeneel et al., FEBS Lett. (1989) 242:211; Murphy et al., FEBS Lett. (1991) 289:4; and Bode et al., Zoology (1996) 99:237) in the part of their sequence involved in the binding of zinc, and can be grouped together as a superfamily, known as the metzincins, on the basis of this sequence similarity. Examples of these proteins include: 1) Angiotensin-converting enzyme (EC 3.4.15.1) (dipeptidyl carboxypeptidase I) (ACE), the enzyme responsible for hydrolyzing angiotensin I to angiotensin II. 2) Mammalian extracellular matrix metalloproteinases (known as matrixins) (Woessner, FASEB J. (1991) 5:2145): MMP-1 (EC 3.4.24.7) (interstitial collagenase), MMP-2 (EC 3.4.24.24) (72 Kd gelatinase), MMP-9 (EC 3.4.24.35) (92 Kd gelatinase), MMP-7 (EC 3.4.24.23) (matrylisin), MMP-8 (EC 3.4.24.34) (neutrophil collagenase), MMP-3 (EC 3.4.24.17) (stromelysin-1), MMP-10 (EC 3.4.24.22) (stromelysin-2), and MMP-11 (stromelysin-3), MMP-12 (EC 3.4.24.65) (macrophage metalloelastase). 3) Endothelin-converting enzyme 1 (EC 3.4.24.71) (ECE-1), which processes the precursor of endothelin to release the active peptide.

A signature pattern which includes the two histidine and the glutamic acid residues is sufficient to detect this superfamily of proteins, having the consensus pattern: [GSTALIVN]-x(2)-H-E-[LIVMFYW]-{DEHRKP}-H-x-[LIVMFYWGSPQ]. The two H's are zinc ligands, and E is the active site residue.

### Example 4

### Differential Expression of Polynucleotides of the Invention: Description of Libraries and Detection of Differential Expression

The relative expression levels of the polynucleotides of the invention was assessed in several libraries prepared from various sources, including cell lines and patient tissue samples. Table 4 provides a summary of these libraries, including the shortened library name (used hereafter), the mRNA source used to prepared the cDNA library, the “nickname” of the library that is used in the tables below (in quotes), and the approximate number of clones in the library.

TABLE 4
Description of cDNA Libraries

| Library (lib#) | Description | Number of Clones in this Clustering |
|---|---|---|
| 1 | Km12 L4 | 307133 |
| | Human Colon Cell Line, High Metastatic Potential | |
| | (derived from Km12C) | |
| | “High Colon” | |
| 2 | Km12C | 284755 |
| | Human Colon Cell Line, Low Metastatic Potential | |
| | “Low Colon” | |
| 3 | MDA-MB-231 | 326937 |
| | Human Breast Cancer Cell Line, High Metastatic | |
| | Potential; micro-metastases in lung | |
| | “High Breast” | |
| 4 | MCF7 | 318979 |
| | Human Breast Cancer Cell, Non Metastatic | |
| | “Low Breast” | |
| 8 | MV-522 | 223620 |
| | Human Lung Cancer Cell Line, High Metastatic | |
| | Potential | |
| | “High Lung” | |
| 9 | UCP-3 | 312503 |
| | Human Lung Cancer Cell Line, Low Metastatic Potential | |
| | “Low Lung” | |
| 12 | Human microvascular endothelial cells (HMEC) - | 41938 |
| | Untreated | |
| | PCR (OligodT) cDNA library | |
| 13 | Human microvascular endothelial cells (HMEC) - bFGF | 42100 |
| | treated | |
| | PCR (OligodT) cDNA library | |
| 14 | Human microvascular endothelial cells (HMEC) - VEGF | 42825 |
| | treated | |
| | PCR (OligodT) cDNA library | |
| 15 | Normal Colon - UC#2 Patient | 34285 |
| | PCR (OligodT) cDNA library | |
| | “Normal Colon Tumor Tissue” | |
| 16 | Colon Tumor - UC#2 Patient | 35625 |
| | PCR (OligodT) cDNA library | |
| | “Normal Colon Tumor Tissue” | |
| 17 | Liver Metastasis from Colon Tumor of UC#2 Patient | 36984 |
| | PCR (OligodT) cDNA library | |
| | “High Colon Metastasis Tissue” | |
| 18 | Normal Colon - UC#3 Patient | 36216 |
| | PCR (OligodT) cDNA library | |
| | “Normal Colon Tumor Tissue” | |
| 19 | Colon Tumor - UC#3 Patient | 41388 |
| | PCR (OligodT) cDNA library | |
| | “High Colon Tumor Tissue” | |
| 20 | Liver Metastasis from Colon Tumor of UC#3 Patient | 30956 |
| | PCR (OligodT) cDNA library | |
| | “High Colon Metastasis Tissue” | |

The KM12L4 and KM12C cell lines are described in Example 1 above. The MDA-MB-231 cell line was originally isolated from pleural effuisions (Cailleau, J. Natl. Cancer. Inst. (1974) 53:661), is of high metastatic potential, and forms poorly differentiated adenocarcinoma grade II in nude mice consistent with breast carcinoma. The MCF7 cell line was derived from a pleural effusion of a breast adenocarcinoma and is non-metastatic. The MV-522 cell line is derived from a human lung carcinoma and is of high metastatic potential. The UCP-3 cell line is a low metastatic human lung carcinoma cell line; the MV-522 is a high metastatic variant of UCP-3. These cell lines are well-recognized in the art as models for the study of human breast and lung cancer (see, e.g., Chandrasekaran et al., Cancer Res. (1979) 39:870 (MDA-MB-231 and MCF-7); Gastpar et al., J Med Chem (1998) 41:4965 (MDA-MB-231 and MCF-7); Ranson et al., Br J Cancer (1998) 77:1586 (MDA-MB-231 and MCF-7); Kuang et al., Nucleic Acids Res (1998) 26:1116 (MDA-MB-231 and MCF-7); Varki et al., Int J Cancer (1987) 40:46 (UCP-3); Varki et al., Tumour Biol. (1990) 11:327; (MV-522 and UCP-3); Varki et al., Anticancer Res. (1990) 10:637; (MV-522); Kelner et al., Anticancer Res (1995) 15:867 (MV-522); and Zhang et al., Anticancer Drugs (1997) 8:696 (MV522)). The samples of libraries 15-20 are derived from two different patients (UC#2, and UC#3).

Each of the libraries is composed of a collection of cDNA clones that in turn are representative of the mRNAs expressed in the indicated mRNA source. In order to facilitate the analysis of the millions of sequences in each library, the sequences were assigned to clusters. The concept of “cluster of clones” is derived from a sorting/grouping of cDNA clones based on their hybridization pattern to a panel of roughly 300 7 bp oligonucleotide probes (see Drmanac et al., Genomics (1996) 37(1):29). Random cDNA clones from a tissue library are hybridized at moderate stringency to 300 7 bp oligonucleotides. Each oligonucleotide has some measure of specific hybridization to that specific clone. The combination of 300 of these measures of hybridization for 300 probes equals the “hybridization signature” for a specific clone. Clones with similar sequence will have similar hybridization signatures. By developing a sorting/grouping algorithm to analyze these signatures, groups of clones in a library can be identified and brought together computationally. These groups of clones are termed “clusters”. Depending on the stringency of the selection in the algorithm (similar to the stringency of hybridization in a classic library cDNA screening protocol), the “purity” of each cluster can be controlled. For example, artifacts of clustering may occur in computational clustering just as artifacts can occur in “wet-lab” screening of a cDNA library with 400 bp cDNA fragments, at even the highest stringency. The stringency used in the implementation of cluster herein provides groups of clones that are in general from the same cDNA or closely related cDNAs. Closely related clones can be a result of different length clones of the same cDNA, closely related clones from highly related gene families, or splice variants of the same cDNA.

Differential expression for a selected cluster was assessed by first determining the number of cDNA clones corresponding to the selected cluster in the first library (Clones in 1^{st}), and the determining the number of cDNA clones corresponding to the selected cluster in the second library (Clones in 2^{nd}). Differential expression of the selected cluster in the first library relative to the second library is expressed as a “ratio” of percent expression between the two libraries. In general, the “ratio” is calculated by: 1) calculating the percent expression of the selected cluster in the first library by dividing the number of clones corresponding to a selected cluster in the first library by the total number of clones analyzed from the first library; 2) calculating the percent expression of the selected cluster in the second library by dividing the number of clones corresponding to a selected cluster in a second library by the total number of clones analyzed from the second library; 3) dividing the calculated percent expression from the first library by the calculated percent expression from the second library. If the “number of clones” corresponding to a selected cluster in a library is zero, the value is set at 1 to aid in calculation. The formula used in calculating the ratio takes into account the “depth” of each of the libraries being compared, i.e., the total number of clones analyzed in each library.

In general, a polynucleotide is said to be significantly differentially expressed between two samples when the ratio value is greater than at least about 2, preferably greater than at least about 3, more preferably greater than at least about 5 , where the ratio value is calculated using the method described above. The significance of differential expression is determined using a z score test (Zar, Biostatistical Analysis, Prentice Hall, Inc., USA, “Differences between Proportions,” pp 296-298 (1974).

Tables 5 to 7 (inserted before the claims) show the number of clones in each of the above libraries that were analyzed for differential expression. Examples of differentially expressed polynucleotides of particular interest are described in more detail below.

### Example 5

### Polynucleotides Differentially Expressed in High Metastatic Potential Breast Cancer Cells Versus Low Metastatic Breast Cancer Cells

A number of polynucleotide sequences have been identified that are differentially expressed between cells derived from high metastatic potential breast cancer tissue and low metastatic breast cancer cells. Expression of these sequences in breast cancer can be valuable in determining diagnostic, prognostic and/or treatment information. For example, sequences that are highly expressed in the high metastatic potential cells can be indicative of increased expression of genes or regulatory sequences involved in the metastatic process. A patient sample displaying an increased level of one or more of these polynucleotides may thus warrant more aggressive treatment. In another example, sequences that display higher expression in the low metastatic potential cells can be associated with genes or regulatory sequences that inhibit metastasis, and thus the expression of these polynucleotides in a sample may warrant a more positive prognosis than the gross pathology would suggest.

The differential expression of these polynucleotides can be used as a diagnostic marker, a prognostic marker, for risk assessment, patient treatment and the like. These polynucleotide sequences can also be used in combination with other known molecular and/or biochemical markers.

The following table summarizes identified polynucleotides with differential expression between high metastatic potential breast cancer cells and low metastatic potential breast cancer cells.

TABLE 8
Differentially expressed polynucleotides: High metastatic potential breast cancer
vs. low metastatic breast cancer cells

| SEQ ID NO. | Differential Expression | Cluster ID | Clones in 1^{st} Library | Clones in 2^{nd} Library | Ratio |
|---|---|---|---|---|---|
| 9 | High Breast > Low Breast (Lib3 > Lib4) | 2623 | 31 | 4 | 7.561356 |
| 42 | High Breast > Low Breast (Lib3 > Lib4) | 307 | 196 | 75 | 2.549721 |
| 52 | High Breast > Low Breast (Lib3 > Lib4) | 19 | 1364 | 525 | 2.534854 |
| 62 | High Breast > Low Breast (Lib3 > Lib4) | 2623 | 31 | 4 | 7.561356 |
| 65 | High Breast > Low Breast (Lib3 > Lib4) | 5749 | 9 | 0 | 8.780930 |
| 66 | High Breast > Low Breast (Lib3 > Lib4) | 6455 | 6 | 0 | 5.853953 |
| 68 | High Breast > Low Breast (Lib3 > Lib4) | 6455 | 6 | 0 | 5.853953 |
| 114 | High Breast > Low Breast (Lib3 > Lib4) | 2030 | 32 | 4 | 7.805271 |
| 123 | High Breast > Low Breast (Lib3 > Lib4) | 3389 | 13 | 2 | 6.341782 |
| 144 | High Breast > Low Breast (Lib3 > Lib4) | 4623 | 12 | 2 | 5.853953 |
| 172 | High Breast > Low Breast (Lib3 > Lib4) | 102 | 278 | 116 | 2.338217 |
| 178 | High Breast > Low Breast (Lib3 > Lib4) | 3681 | 10 | 1 | 9.756589 |
| 214 | High Breast > Low Breast (Lib3 > Lib4) | 3900 | 8 | 1 | 7.805271 |
| 219 | High Breast > Low Breast (Lib3 > Lib4) | 3389 | 13 | 2 | 6.341782 |
| 223 | High Breast > Low Breast (Lib3 > Lib4) | 1399 | 19 | 7 | 2.648217 |
| 258 | High Breast > Low Breast (Lib3 > Lib4) | 4837 | 10 | 0 | 9.756589 |
| 317 | High Breast > Low Breast (Lib3 > Lib4) | 1577 | 25 | 3 | 8.130490 |
| 379 | High Breast > Low Breast (Lib3 > Lib4) | 260 | 27 | 2 | 13.17139 |
| 4 | Low Breast > High Breast (Lib4 > Lib3) | 3706 | 22 | 4 | 5.637215 |
| 39 | Low Breast > High Breast (Lib4 > Lib3) | 4016 | 6 | 0 | 6.149690 |
| 74 | Low Breast > High Breast (Lib4 > Lib3) | 6268 | 18 | 3 | 6.149690 |
| 81 | Low Breast > High Breast (Lib4 > Lib3) | 40392 | 8 | 1 | 8.199586 |
| 130 | Low Breast > High Breast (Lib4 > Lib3) | 13183 | 7 | 0 | 7.174638 |
| 157 | Low Breast > High Breast (Lib4 > Lib3) | 5417 | 9 | 0 | 9.224535 |
| 162 | Low Breast > High Breast (Lib4 > Lib3) | 9685 | 7 | 0 | 7.174638 |
| 183 | Low Breast > High Breast (Lib4 > Lib3) | 7337 | 16 | 3 | 5.466391 |
| 202 | Low Breast > High Breast (Lib4 > Lib3) | 6124 | 9 | 1 | 9.224535 |
| 298 | Low Breast > High Breast (Lib4 > Lib3) | 1037 | 22 | 4 | 5.637215 |
| 338 | Low Breast > High Breast (Lib4 > Lib3) | 689 | 36 | 17 | 2.170478 |
| 384 | Low Breast > High Breast (Lib4 > Lib3) | 697 | 72 | 30 | 2.459876 |
| 386 | Low Breast > High Breast (Lib4 > Lib3) | 4568 | 9 | 0 | 9.224535 |
| 388 | Low Breast > High Breast (Lib4 > Lib3) | 5622 | 13 | 2 | 6.662164 |

### Example 6

### Polynucleotides Differentially Expressed in High Metastatic Potential Lung Cancer Cells Versus Low Metastatic Lung Cancer Cells

A number of polynucleotide sequences have been identified that are differentially expressed between cells derived from high metastatic potential lung cancer tissue and low metastatic lung cancer cells. Expression of these sequences in lung cancer tissue can be valuable in determining diagnostic, prognostic and/or treatment information. For example, sequences that are highly expressed in the high metastatic potential cells are associated can be indicative of increased expression of genes or regulatory sequences involved in the metastatic process. A patient sample displaying an increased level of one or more of these polynucleotides may thus warrant more aggressive treatment. In another example, sequences that display higher expression in the low metastatic potential cells can be associated with genes or regulatory sequences that inhibit metastasis, and thus the expression of these polynucleotides in a sample may warrant a more positive prognosis than the gross pathology would suggest.

The differential expression of these polynucleotides can be used as a diagnostic marker, a prognostic marker, for risk assessment, patient treatment and the like. These polynucleotide sequences can also be used in combination with other known molecular and/or biochemical markers.

The following table summarizes identified polynucleotides with differential expression between high metastatic potential lung cancer cells and low metastatic potential lung cancer cells:

TABLE 9
Differentially expressed polynucleotides: High metastatic potential lung cancer
vs. low metastatic lung cancer cells

| SEQ ID NO. | Differential Expression | Cluster ID | Clones in 1^{st} Library | Clones in 2^{nd} Library | Ratio |
|---|---|---|---|---|---|
| 400 | High Lung > Low Lung (Lib8 > Lib9) | 14929 | 23 | 16 | 2.008868 |
| 9 | High Lung > Low Lung (Lib8 > Lib9) | 2623 | 6 | 1 | 8.384840 |
| 34 | High Lung > Low Lung (Lib8 > Lib9) | 5832 | 5 | 0 | 6.987366 |
| 42 | High Lung > Low Lung (Lib8 > Lib9) | 307 | 79 | 27 | 4.088903 |
| 62 | High Lung > Low Lung (Lib8 > Lib9) | 2623 | 6 | 1 | 8.384840 |
| 74 | High Lung > Low Lung (Lib8 > Lib9) | 6268 | 5 | 0 | 6.987366 |
| 106 | High Lung > Low Lung (Lib8 > Lib9) | 10717 | 8 | 0 | 11.17978 |
| 119 | High Lung > Low Lung (Lib8 > Lib9) | 8 | 1355 | 122 | 15.52111 |
| 361 | High Lung > Low Lung (Lib8 > Lib9) | 1120 | 5 | 0 | 6.987366 |
| 369 | High Lung > Low Lung (Lib8 > Lib9) | 2790 | 6 | 0 | 8.384840 |
| 371 | High Lung > Low Lung (Lib8 > Lib9) | 8847 | 6 | 1 | 8.384840 |
| 379 | High Lung > Low Lung (Lib8 > Lib9) | 260 | 15 | 0 | 20.96210 |
| 395 | High Lung > Low Lung (Lib8 > Lib9) | 13538 | 9 | 1 | 12.57726 |
| 135 | Low Lung > High Lung (Lib9 > Lib8) | 36313 | 30 | 1 | 21.46731 |
| 154 | Low Lung > High Lung (Lib9 > Lib8) | 5345 | 27 | 6 | 3.220097 |
| 160 | Low Lung > High Lung (Lib9 > Lib8) | 4386 | 21 | 3 | 5.009039 |
| 260 | Low Lung > High Lung (Lib9 > Lib8) | 4141 | 27 | 4 | 4.830145 |
| 308 | Low Lung > High Lung (Lib9 > Lib8) | 15855 | 213 | 12 | 12.70149 |
| 323 | Low Lung > High Lung (Lib9 > Lib8) | 5257 | 25 | 5 | 3.577885 |
| 349 | Low Lung > High Lung (Lib9 > Lib8) | 2797 | 14 | 1 | 10.01807 |
| 381 | Low Lung > High Lung (Lib9 > Lib8) | 2428 | 19 | 2 | 6.797982 |

### Example 7

### Polynucleotides Differentially Expressed in High Metastatic Potential Colon Cancer Cells Versus Low Metastatic Colon Cancer Cells

A number of polynucleotide sequences have been identified that are differentially expressed between cells derived from high metastatic potential colon cancer tissue and low metastatic colon cancer cells. Expression of these sequences in colon cancer tissue can be valuable in determining diagnostic, prognostic and/or treatment information. For example, sequences that are highly expressed in the high metastatic potential cells can be indicative of increased expression of genes or regulatory sequences involved in the metastatic process. A patient sample displaying an increased level of one or more of these polynucleotides may thus warrant more aggressive treatment. In another example, sequences that display higher expression in the low metastatic potential cells can be associated with genes or regulatory sequences that inhibit metastasis, and thus the expression of these polynucleotides in a sample may warrant a more positive prognosis than the gross pathology would suggest.

The differential expression of these polynucleotides can be used as a diagnostic marker, a prognostic marker, for risk assessment, patient treatment and the like. These polynucleotide sequences can also be used in combination with other known molecular and/or biochemical markers.

The following table summarizes identified polynucleotides with differential expression between high metastatic potential colon cancer cells and low metastatic potential colon cancer cells:

TABLE 10
Differentially expressed polynucleotides: High metastatic potential colon cancer
vs. low metastatic colon cancer cells

| SEQ ID NO. | Differential Expression | Cluster ID | Clones in 1^{st} Library | Clones in 2^{nd} Library | Ratio |
|---|---|---|---|---|---|
| 1 | High Colon > Low Colon (Lib1 > Lib2) | 6660 | 7 | 0 | 6.489973 |
| 176 | High Colon > Low Colon (Lib1 > Lib2) | 3765 | 19 | 6 | 2.935940 |
| 241 | High Colon > Low Colon (Lib1 > Lib2) | 4275 | 11 | 2 | 5.099264 |
| 362 | High Colon > Low Colon (Lib1 > Lib2) | 6420 | 8 | 0 | 7.417112 |
| 374 | High Colon > Low Colon (Lib1 > Lib2) | 6420 | 8 | 0 | 7.417112 |
| 39 | Low Colon > High Colon (Lib2 > Lib1) | 4016 | 14 | 5 | 3.020043 |
| 97 | Low Colon > High Colon (Lib2 > Lib1) | 945 | 21 | 9 | 2.516702 |
| 134 | Low Colon > High Colon (Lib2 > Lib1) | 2464 | 19 | 5 | 4.098630 |
| 317 | Low Colon > High Colon (Lib2 > Lib1) | 1577 | 40 | 12 | 3.595289 |
| 357 | Low Colon > High Colon (Lib2 > Lib1) | 4309 | 13 | 4 | 3.505407 |

### Example 8

### Polynucleotides Differentially Expressed at Higher Levels in High Metastatic Potential Colon Cancer Patient Tissue Versus Normal Patient Tissue

A number of polynucleotide sequences have been identified that are differentially expressed between cells derived from high metastatic potential colon cancer tissue and normal tissue. Expression of these sequences in colon cancer tissue can be valuable in determining diagnostic, prognostic and/or treatment information. For example, sequences that are highly expressed in the high metastatic potential cells are associated can be indicative of increased expression of genes or regulatory sequences involved in the advanced disease state which involves processes such as angiogenesis, dedifferentiation, cell replication, and metastasis. A patient sample displaying an increased level of one or more of these polynucleotides may thus warrant more aggressive treatment.

The differential expression of these polynucleotides can be used as a diagnostic marker, a prognostic marker, for risk assessment, patient treatment and the like. These polynucleotide sequences can also be used in combination with other known molecular and/or biochemical markers.

The following table summarizes identified polynucleotides with differential expression between high metastatic potential colon cancer cells and normal colon cells:

TABLE 11
Differentially expressed polynucleotides: High metastatic potential colon tissue
vs. normal colon tissue

| SEQ ID NO. | Differential Expression | Cluster ID | Clones in 1^{st} Library | Clones in 2^{nd} Library | Ratio |
|---|---|---|---|---|---|
| 52 | High Colon Metastasis Tissue > Normal | 19 | 10 | 0 | 11.6991 |
| | Colon Tissue of UC#3 (Lib20 > Lib18) | | | | 8 |
| 52 | High Colon Metastasis Tissue > Normal | 19 | 13 | 2 | 6.02564 |
| | Tissue in UC#2 (Lib17 > Lib15) | | | | 6 |
| 172 | High Colon Metastasis Tissue > Normal | 102 | 65 | 22 | 2.73893 |
| | Tissue in UC#2 (Lib17 > Lib15) | | | | 0 |

### Example 9

### Polynucleotides Differentially Expressed at Higher Levels in High Colon Tumor Potential Patient Tissue Versus Metastasized Colon Cancer Patient Tissue

A number of polynucleotide sequences have been identified that are differentially expressed between cells derived from high tumor potential colon cancer tissue and cells derived from high metastatic potential colon cancer cells. Expression of these sequences in colon cancer tissue can be valuable in determining diagnostic, prognostic and/or treatment information associated with the transformation of precancerous tissue to malignant tissue. This information can be useful in the prevention of achieving the advanced malignant state in these tissues, and can be important in risk assessment for a patient.

The following table summarizes identified polynucleotides with differential expression between high tumor potential colon cancer tissue and cells derived from high metastatic potential colon cancer cells:

TABLE 12
Differentially expressed polynucleotides: High tumor potential colon tissue vs.
metastatic colon tissue

| SEQ ID NO. | Differential Expression | Cluster ID | Clones in 1^{st} Library | Clones in 2^{nd} Library | Ratio |
|---|---|---|---|---|---|
| 52 | High Colon Tumor Tissue > Metastasis | 19 | 69 | 10 | 5.16082 |
| | Tissue of UC#3 (Lib19 > Lib20) | | | | 9 |
| 119 | High Colon Tumor Tissue > Metastasis | 8 | 14 | 1 | 10.4712 |
| | Tissue of UC#3 (Lib19 > Lib20) | | | | 4 |
| 172 | High Colon Tumor Tissue > Metastasis | 102 | 43 | 10 | 3.21616 |
| | Tissue of UC#3 (Lib19 > Lib20) | | | | 8 |

### Example 10

### Polynucleotides Differentially Expressed at Higher Levels in High Tumor Potential Colon Cancer Patient Tissue Versus Normal Patient Tissue

A number of polynucleotide sequences have been identified that are differentially expressed between cells derived from high tumor potential colon cancer tissue and normal tissue. Expression of these sequences in colon cancer tissue can be valuable in determining diagnostic, prognostic and/or treatment information associated with the prevention of achieving the malignant state in these tissues, and can be important in risk assessment for a patient. For example, sequences that are highly expressed in the potential colon cancer cells are associated with or can be indicative of increased expression of genes or regulatory sequences involved in early tumor progression. A patient sample displaying an increased level of one or more of these polynucleotides may thus warrant closer attention or more frequent screening procedures to catch the malignant state as early as possible.

The following table summarizes identified polynucleotides with differential expression between high metastatic potential colon cancer cells and normal colon cells:

TABLE 13
Differentially expressed polynucleotides: High tumor potential colon tissue vs.
normal colon tissue

| SEQ ID NO. | Differential Expression | Cluster ID | Clones in 1^{st} Library | Clones in 2^{nd} Library | Ratio |
|---|---|---|---|---|---|
| 52 | High Colon Tumor Tissue > Normal | 19 | 13 | 2 | 6.25550 |
| | Tissue of UC#2 (Lib16 > Lib15) | | | | 8 |
| 288 | High Colon Tumor Tissue > Normal | 1267 | 7 | 0 | 6.12525 |
| | Tissue of UC#2 (Lib16 > Lib15) | | | | 3 |
| 52 | High Colon Tumor Tissue > Normal | 19 | 69 | 0 | 60.3775 |
| | Tissue of UC#3 (Lib19 > Lib18) | | | | 0 |
| 119 | High Colon Tumor Tissue > Normal | 8 | 14 | 1 | 12.2505 |
| | Tissue of UC#3 (Lib19 > Lib18) | | | | 0 |
| 172 | High Colon Tumor Tissue > Normal | 102 | 43 | 7 | 5.37522 |
| | Tissue of UC#3 (Lib19 > Lib18) | | | | 2 |

### Example 11

### Polynucleotides Differentially Expressed Across Multiple Libraries

A number of polynucleotide sequences have been identified that are differentially expressed between cancerous cells and normal cells across all three tissue types tested (i.e., breast, colon, and lung). Expression of these sequences in a tissue or any origin can be valuable in determining diagnostic, prognostic and/or treatment information associated with the prevention of achieving the malignant state in these tissues, and can be important in risk assessment for a patient. These polynucleotides can also serve as non-tissue specific markers of, for example, risk of metastasis of a tumor. The following table summarizes identified polynucleotides that were differentially expressed but without tissue type-specificity in the breast, colon, and lung libraries tested.

TABLE 14
Polynucleotides Differentially Expressed Across Multiple Library Comparisons

| SEQ ID NO. | Differential Expression | Cluster ID | Clones in 1^{st} Library | Clones in 2^{nd} Library | Ratio |
|---|---|---|---|---|---|
| 9 | High Breast > Low Breast (Lib3 > Lib4) | 2623 | 31 | 4 | 7.561356 |
| | High Lung > Low Lung (Lib8 > Lib9) | 2623 | 6 | 1 | 8.384840 |
| 39 | Low Breast > High Breast (Lib4 > Lib3) | 4016 | 6 | 0 | 6.149690 |
| | Low Colon > High Colon (Lib2 > Lib1) | 4016 | 14 | 5 | 3.020043 |
| 42 | High Breast > Low Breast (Lib3 > Lib4) | 307 | 196 | 75 | 2.549721 |
| | High Lung > LowLung (Lib8 > Lib9) | 307 | 79 | 27 | 4.088903 |
| 52 | High Breast > Low Breast (Lib3 > Lib4) | 19 | 1364 | 525 | 2.534854 |
| | High Colon Metastasis Tissue > Normal | 19 | 10 | 0 | 11.69918 |
| | Colon Tissue of UC#3 (Lib20 > Lib 18) | | | | |
| | High Colon Metastasis Tissue > Normal | 19 | 13 | 2 | 6.025646 |
| | Tissue in UC#2 (Lib17 > Lib15) | | | | |
| | High Colon Tumor Tissue > Metastasis | 19 | 69 | 10 | 5.160829 |
| | Tissue of UC#3 (Lib19 > Lib20) | | | | |
| | High Colon Tumor Tissue > Normal | 19 | 13 | 2 | 6.255508 |
| | Tissue of UC#2 (Lib16 > Lib15) | | | | |
| | High Colon Tumor Tissue > Normal | 19 | 69 | 0 | 60.37750 |
| | Tissue of UC#3 (Lib19 > Lib18) | | | | |
| 62 | High Breast > Low Breast (Lib3 > Lib4) | 2623 | 31 | 4 | 7.561356 |
| | High Lung > Low Lung (Lib8 > Lib9) | 2623 | 6 | 1 | 8.384840 |
| 74 | High Lung > Low Lung (Lib8 > Lib9) | 6268 | 5 | 0 | 6.987366 |
| | Low Breast > High Breast (Lib4 > Lib3) | 6268 | 18 | 3 | 6.149690 |
| 119 | High Colon Tumor Tissue > Metastasis | 8 | 14 | 1 | 10.47124 |
| | Tissue of UC#3 (Lib19 > Lib20) | | | | |
| | High Colon Tumor Tissue > Normal | 8 | 14 | 1 | 12.25050 |
| | Tissue of UC#3 (Lib19 > Lib18) | | | | |
| | High Lung > Low Lung (Lib8 > Lib9) | 8 | 1355 | 122 | 15.52111 |
| 172 | High Breast> Low Breast (Lib3 > Lib4) | 102 | 278 | 116 | 2.338217 |
| | High Colon Metastasis Tissue > Normal | 102 | 65 | 22 | 2.738930 |
| | Tissue in UC#2 (Lib17 > Lib15) | | | | |
| | High Colon Tumor Tissue > Metastasis | 102 | 43 | 10 | 3.216168 |
| | Tissue of UC#3 (Lib19 > Lib20) | | | | |
| | High Colon Tumor Tissue > Normal | 102 | 43 | 7 | 5.375222 |
| | Tissue of UC#3 (Lib19 > Lib18) | | | | |
| 317 | High Breast > Low Breast (Lib3 > Lib4) | 1577 | 25 | 3 | 8.130490 |
| | Low Colon > High Colon (Lib2 > Lib1) | 1577 | 40 | 12 | 3.595289 |
| 379 | High Breast > Low Breast (Lib3 > Lib4) | 260 | 27 | 2 | 13.17139 |
| | High Lung > Low Lung (Lib8 > Lib9) | 260 | 15 | 0 | 20.96210 |

### Example 12

### Polynucleotides Exhibiting Colon-Specific Expression

The cDNA libraries described herein were also analyzed to identify those polynucleotides that were specifically expressed in colon cells or tissue, i.e., the polynucleotides were identified in libraries prepared from colon cell lines or tissue, but not in libraries of breast or lung origin. The polynucleotides that were expressed in a colon cell line and/or in colon tissue, but were present in the breast or lung cDNA libraries described herein, are shown in Table 15.

TABLE 15
Polynucleotides specifically expressed in colon cells.

| | | Clones in | Clones in |
|---|---|---|---|
| SEQ ID | | 1^{st} | 2^{nd} |
| NO. | Cluster | Library | Library |
| 5 | 36535 | 2 | 0 |
| 13 | 27250 | 2 | 0 |
| 19 | 16283 | 3 | 0 |
| 24 | 16918 | 4 | 0 |
| 26 | 40108 | 2 | 0 |
| 32 | 32663 | 1 | 1 |
| 43 | 39833 | 2 | 0 |
| 47 | 18957 | 3 | 0 |
| 48 | 39508 | 2 | 0 |
| 56 | 7005 | 8 | 2 |
| 58 | 18957 | 3 | 0 |
| 59 | 18957 | 3 | 0 |
| 60 | 16283 | 3 | 0 |
| 64 | 13238 | 4 | 1 |
| 70 | 39442 | 2 | 0 |
| 71 | 17036 | 4 | 0 |
| 73 | 7005 | 8 | 2 |
| 83 | 11476 | 6 | 0 |
| 86 | 39425 | 2 | 0 |
| 94 | 21847 | 2 | 1 |
| 100 | 16731 | 3 | 1 |
| 101 | 12439 | 4 | 0 |
| 113 | 17055 | 4 | 0 |
| 120 | 67907 | 1 | 0 |
| 121 | 12081 | 4 | 0 |
| 124 | 39174 | 2 | 0 |
| 126 | 8210 | 2 | 6 |
| 128 | 40455 | 2 | 0 |
| 139 | 22195 | 3 | 0 |
| 143 | 86859 | 1 | 0 |
| 150 | 8672 | 4 | 4 |
| 153 | 16977 | 4 | 0 |
| 156 | 17036 | 4 | 0 |
| 159 | 40044 | 2 | 0 |
| 161 | 40044 | 2 | 0 |
| 163 | 22155 | 3 | 0 |
| 166 | 15066 | 4 | 0 |
| 170 | 11465 | 5 | 0 |
| 176 | 3765 | 19 | 6 |
| 181 | 86110 | 1 | 0 |
| 182 | 39648 | 2 | 0 |
| 185 | 17076 | 4 | 0 |
| 186 | 22794 | 2 | 0 |
| 187 | 39171 | 2 | 0 |
| 194 | 40455 | 2 | 0 |
| 199 | 16317 | 3 | 0 |
| 210 | 39186 | 2 | 0 |
| 211 | 40122 | 2 | 0 |
| 218 | 26295 | 2 | 0 |
| 222 | 4665 | 5 | 9 |
| 226 | 82498 | 1 | 0 |
| 227 | 35702 | 2 | 0 |
| 229 | 39648 | 2 | 0 |
| 231 | 85064 | 1 | 0 |
| 234 | 39391 | 2 | 0 |
| 236 | 39498 | 2 | 0 |
| 242 | 22113 | 3 | 0 |
| 247 | 19255 | 2 | 0 |
| 252 | 22814 | 3 | 0 |
| 253 | 39563 | 2 | 0 |
| 254 | 39420 | 2 | 0 |
| 257 | 39412 | 2 | 0 |
| 261 | 38085 | 2 | 0 |
| 265 | 40054 | 1 | 0 |
| 266 | 39423 | 2 | 0 |
| 267 | 39453 | 2 | 0 |
| 270 | 78091 | 1 | 0 |
| 276 | 39168 | 2 | 0 |
| 277 | 39458 | 2 | 0 |
| 278 | 14391 | 3 | 1 |
| 279 | 39195 | 2 | 0 |
| 282 | 12977 | 5 | 0 |
| 284 | 14391 | 3 | 1 |
| 290 | 16347 | 4 | 0 |
| 293 | 39478 | 2 | 0 |
| 294 | 39392 | 2 | 0 |
| 297 | 39180 | 2 | 0 |
| 299 | 6867 | 7 | 3 |
| 301 | 41633 | 1 | 1 |
| 302 | 23218 | 3 | 0 |
| 303 | 39380 | 2 | 0 |
| 309 | 84328 | 1 | 0 |
| 314 | 14367 | 3 | 0 |
| 320 | 39886 | 2 | 0 |
| 324 | 9061 | 5 | 2 |
| 327 | 16653 | 3 | 1 |
| 328 | 16985 | 4 | 0 |
| 329 | 12977 | 5 | 0 |
| 330 | 9061 | 5 | 2 |
| 333 | 16392 | 3 | 0 |
| 342 | 39486 | 2 | 0 |
| 344 | 6874 | 6 | 3 |
| 345 | 6874 | 6 | 3 |
| 353 | 11494 | 4 | 0 |
| 354 | 17062 | 3 | 0 |
| 355 | 16245 | 4 | 0 |
| 356 | 83103 | 1 | 0 |
| 358 | 13072 | 4 | 1 |
| 366 | 14364 | 1 | 0 |
| 368 | 84182 | 1 | 0 |
| 372 | 56020 | 1 | 0 |
| 389 | 7514 | 5 | 3 |
| 391 | 7570 | 5 | 3 |
| 393 | 23210 | 3 | 0 |

In addition to the above, SEQ ID NOS:159 and 161 were each present in one clone in each of Lib16 (Normal Colon Tumor Tissue), and SEQ ID NOS:344 and 345 were each present in one clone in Libl7 (High Colon Metastasis Tissue). No clones corresponding to the colon-specific polynucleotides in the table above were present in any of Libraries 3, 4, 8, or 9. The polynucleotide provided above can be used as markers of cells of colon origin, and find particular use in reference arrays, as described above.

### Example 13

### Identification of Contiguous Sequences Having a Polynucleotide of the Invention

The novel polynucleotides were used to screen publicly available and proprietary databases to determine if any of the polynucleotides of SEQ ID NOS:1-404 would facilitate identification of a contiguous sequence, e.g, the polynucleotides would provide sequence that would result in 5′ extension of another DNA sequence, resulting in production of a longer contiguous sequence composed of the provided polynucleotide and the other DNA sequence(s). Contiging was performed using the AssemblyLign program with the following parameters: 1) Overlap: Minimum Overlap Length: 30;% Stringency: 50; Minimum Repeat Length: 30; Alignment: gap creation penalty: 1.00, gap extension penalty: 1.00; 2) Consensus: % Base designation threshold: 80.

Using these parameters, 44 polynucleotides provided contiged sequences. These contiged sequences are provided as SEQ ID NOS:801-844. The contiged sequences can be correlated with the sequences of SEQ ID NOS:1-404 upon which the contiged sequences are based by identifying those sequences of SEQ ID NOS:1-404 and the contiged sequences of SEQ ID NOS:801-844 that share the same clone name in Table 1. It should be noted that of these 44 sequences that provided a contiged sequence, the following members of that group of 44 did not contig using the overlap settings indicated in parentheses (Stringency/Overlap): SEQ ID NO:804 (30%/10); SEQ ID NO:810 (20%/20); SEQ ID NO:812 (30%/10); SEQ ID NO:814 (40%/20); SEQ ID NO:816 (30%/10); SEQ ID NO:832 (30%/10); SEQ ID NO:840 (20%/20); SEQ ID NO:841 (40%/20). To generalize, the indicated polynucleotides did not contig using a minimum 20% stringency, 10 overlap. There was a corresponding increase in the number of degenerate codons in these sequences.

The contiged sequences (SEQ ID NO:801-844) thus represent longer sequences that encompass a polynucleotide sequence of the invention. The contiged sequences were then translated in all three reading frames to determine the best alignment with individual sequences using the BLAST programs as described above for SEQ ID NOS:1-404 and the validation sequences SEQ ID NOS:405-800. Again the sequences were masked using the XBLAST profram for masking low complexity as described above in Example 1 (Table 2). Several of the contiged sequences were found to encode polypeptides having characteristics of a polypeptide belonging to a known protein families (and thus represent new members of these protein families) and/or comprising a known functional domain (Table 16). Thus the invention encompasses fragments, fusions, and variants of such polynucleotides that retain biological activity associated with the protein family and/or functional domain identified herein.

TABLE 16
Profile hits using contiged sequences

| SEQ ID | | | Start | |
|---|---|---|---|---|
| NO. | Sequence Name | Profile | (Stop) | Score |
| 809 | Contig_RTA00000177AF.n.18.3. | ATPases | 778 | 6040 |
| | Seq_THC 123051 | | (1612) | |
| 824 | Contig_RTA00000187AF.g.24.1. | homeobox | 531 | 12080 |
| | Sec_THC168636 | | (707) | |
| 824 | Contig_RTA00000187AF.g.24.1. | MAP kinase | 769 | 5784 |
| | Seq_THC 168636 | kinase | (1494) | |
| 833 | Contig_RTA00000190AF.j.4.1. | protein kinase | 170 | 5027 |
| | Seq_THC228776 | | (1010) | |
| 833 | Contig_RTA00000190AF.j.4.1. | protein kinase | 170 | 5027 |
| | Seq_THC228776 | | (1010) | |

All stop/start sequences are provided in the forward direction.

The profiles for the ATPases (AAA) and protein kinase families are described above in Example 2. The homeobox and MAP kinase kinase protein families are described further below.

Homeobox Domain.

The ‘homeobox’ is a protein domain of 60 amino acids (Gehring In: Guidebook to the Homeobox Genes, Duboule D., Ed., pp1-10, Oxford University Press, Oxford, (1994); Buerglin In: Guidebook to the Homeobox Genes, pp25-72, Oxford University Press, Oxford, (1994); Gehring Trends Biochem. Sci. (1992) 1 7:277-280; Gehring et al Annu. Rev. Genet. (1986) 20:147-173; Schofield Trends Neurosci. (1987) 10:3-6; http://copan.bioz.unibas.ch/homeo.html) first identified in number of Drosophila homeotic and segmentation proteins. It is extremely well conserved in many other animals, including vertebrates. This domain binds DNA through a helix-turn-helix type of structure. Several proteins that contain a homeobox domain play an important role in development. Most of these proteins are sequence-specific DNA-binding transcription factors. The homeobox domain is also very similar to a region of the yeast mating type proteins. These are sequence-specific DNA-binding proteins that act as master switches in yeast differentiation by controlling gene expression in a cell type-specific fashion.

A schematic representation of the homeobox domain is shown below. The helix-turn-helix region is shown by tne symbols ‘H’ (for helix), and ‘t’ (for turn).

xxxxxxxxxxxxxxxxxxxxxxxxxxxxxxHHHHHHHHtttHHHHHHHHHxxxxxxxxxx
1 60

The pattern detects homeobox sequences 24 residues long and spans positions 34 to 57 of the homeobox domain. The consensus pattern is as follows: [LIVMFYG]-[ASLVR]-x(2)-[LIVMSTACN]-x-[LIVM]-x(4)-[LIV]-[RKNQESTAIY]-[LIVFSTNKH]-W-[FYVC]-x-[NDQTAH]-x(5)-[RKNAIMW].

MAP Kinase Kinase (MAPKK).

MAP kinases (MAPK) are involved in signal transduction, and are important in cell cycle and cell growth controls. The MAP kinase kinases (MAPKK) are dual-specificity protein kinases which phosphorylate and activate MAP kinases. MAPKK homologues have been found in yeast, invertebrates, amphibians, and mammals. Moreover, the MAPKK/MAPK phosphorylation switch constitutes a basic module activated in distinct pathways in yeast and in vertebrates. MAPKK regulation studies have led to the discovery of at least four MAPKK convergent pathways in higher organisms. One of these is similar to the yeast pheromone response pathway which includes the ste11 protein kinase. Two other pathways require the activation of either one or both of the serine/threonine kinase-encoded oncogenes c-Raf-1 and c-Mos. Additionally, several studies suggest a possible effect of the cell cycle control regulator cyclin-dependent kinase 1 (cdc2) on MAPKK activity. Finally, MAPKKs are apparently essential transducers through which signals must pass before reaching the nucleus. For review, see, e.g., Biologique Biol Cell (1993) 79:193-207; Nishida et al., Trends Biochem Sci (1993) 18:128-31; Ruderman Curr Opin Cell Biol (1993) 5:207-13; Dhanasekaran et al., Oncogene (1998) 17:1447-55; Kiefer et al., Biochem Soc Trans (1997) 25:491-8; and Hill, Cell Signal (1996) 8:533-44.

Those skilled in the art will recognize, or be able to ascertain, using not more than routine experimentation, many equivalents to the specific embodiments of the invention described herein. Such specific embodiments and equivalents are intended to be encompassed by the following claims.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Deposit Information:

The following materials were deposited with the American Type Culture Collection: CMCC=(Chiron Master Culture Collection)

Cell Lines Deposited with ATCC

| | | ATCC | CMCC |
|---|---|---|---|
| Cell Line | Deposit Date | Accession No. | Accession No. |
| KM12L4-A | Mar. 19, 1998 | CRL-12496 | 11606 |
| Km12C | May 15, 1998 | CRL-12533 | 11611 |
| MDA-MB-231 | May 15, 1998 | CRL-12532 | 10583 |
| MCF-7 | Oct. 9, 1998 | CRL-12584 | 10377 |

CMCC = (Chiron Master Culture Collection)

CDNA Library Deposits
cDNA Library ES1 - ATCC#
Deposit Date - Dec. 22, 1998

| Clone Name | Cluster ID | Sequence Name |
|---|---|---|
| M00001395A:C03 | 4016 | 79.A1.sp6:130016.Seq |
| M00001395A:C03 | 4016 | RTA00000118A.c.4.1 |
| M00001449A:D12 | 3681 | RTA00000131A.g.15.2 |
| M00001449A:D12 | 3681 | 79.E1.sp6:130064.Seq |
| M00001452A:D08 | 1120 | 79.C2.sp6:130041.Seq |
| M00001452A:D08 | 1120 | RTA00000118A.p.15.3 |
| M00001513A:B06 | 4568 | 79.D4.sp6:130055.Seq |
| M00001513A:B06 | 4568 | RTA00000122A.d.15.3 |
| M00001517A:B07 | 4313 | 79.F4.sp6:130079.Seq |
| M00001517A:B07 | 4313 | RTA00000122A.n.3.1 |
| M00001533A:C11 | 2428 | RTA00000123A.l.21.1 |
| M00001533A:C11 | 2428 | 79.A5.sp6:130020.Seq |
| M00001533A:C11 | 2428 | RTA00000123A.l.21.1.Seq_THC205063 |
| M00001542A:A09 | 22113 | 79.F5.sp6:130080.Seq |
| M00001542A:A09 | 22113 | RTA00000125A.c.7.1 |
| M00001343C:F10 | 2790 | 80.E1.sp6:130256.Seq |
| M00001343C:F10 | 2790 | RTA00000177AF.e.2.1.Seq_THC229461 |
| M00001343C:F10 | 2790 | RTA00000177AF.e.2.1 |
| M00001343D:H07 | 23255 | 100.C1.sp6:131446.Seq |
| M00001343D:H07 | 23255 | RTA00000177AF.e.14.3.Seq_THC228776 |
| M00001343D:H07 | 23255 | 80.F1.sp6:130268.Seq |
| M00001343D:H07 | 23255 | RTA00000177AF.e.14.3 |
| M00001345A:E01 | 6420 | 172.E1.sp6:133925.Seq |
| M00001345A:E01 | 6420 | RTA00000177AF.f.10.3 |
| M00001345A:E01 | 6420 | RTA00000177AF.f.10.3.Seq_THC226443 |
| M00001345A:E01 | 6420 | 80.G1.sp6:130280.Seq |
| M00001347A:B10 | 13576 | 80.D2.sp6:130245.Seq |
| M00001347A:B10 | 13576 | 100.E1.sp6:131470.Seq |
| M00001347A:B10 | 13576 | RTA00000177AF.g.16.1 |
| M00001353A:G12 | 8078 | 80.E3.sp6:130258.Seq |
| M00001353A:G12 | 8078 | RTA00000177AR.l.13.1 |
| M00001353A:G12 | 8078 | 172.C3.sp6:133903.Seq |
| M00001353D:D10 | 14929 | RTA00000177AF.m.1.2 |
| M00001353D:D10 | 14929 | 80.F3.sp6:130270.Seq |
| M00001353D:D10 | 14929 | 172.D3.sp6:133915.Seq |
| M00001361A:A05 | 4141 | 80.B4.sp6:130223.Seq |
| M00001361A:A05 | 4141 | RTA00000177AF.p.20.3 |
| M00001362B:D10 | 5622 | 80.D4.sp6:130247.Seq |
| M00001362B:D10 | 5622 | RTA00000178AF.a.11.1 |
| M00001362C:H11 | 945 | RTA00000178AR.a.20.1 |
| M00001362C:H11 | 945 | 100.E4.sp6:131473.Seq |
| M00001362C:H11 | 945 | 80.E4.sp6:130259.Seq |
| M00001362C:H11 | 945 | 180.C2.sp6:135940.Seq |
| M00001376B:G06 | 17732 | RTA00000178AR.i.2.2 |
| M00001376B:G06 | 17732 | 80.B5.sp6:130224.Seq |
| M00001387A:C05 | 2464 | 80.D6.sp6:130249.Seq |
| M00001387A:C05 | 2464 | RTA00000178AF.n.18.1 |
| M00001412B:B10 | 8551 | RTA00000179AF.p.21.1 |
| M00001412B:B10 | 8551 | 80.G7.sp6:130286.Seq |
| M00001415A:H06 | 13538 | 80.B8.sp6:130227.Seq |
| M00001415A:H06 | 13538 | RTA00000180AF.a.24.1 |
| M00001416B:H11 | 8847 | 80.C8.sp6:130239.Seq |
| M00001416B:H11 | 8847 | RTA00000180AF.b.16.1 |
| M00001429D:D07 | 40392 | RTA00000180AF.j.8.1 |
| M00001429D:D07 | 40392 | 80.H9.sp6:130300.Seq |
| M00001448D:H01 | 36313 | 80.A11.sp6:130218.Seq |
| M00001448D:H01 | 36313 | RTA00000181AF.e.23.1 |
| M00001463C:B11 | 19 | RTA00000182AF.b.7.1 |
| M00001463C:B11 | 19 | 89.D1.sp6:130703.Seq |
| M00001470A:B10 | 1037 | 89.F2.sp6:130728.Seq |
| M00001470A:B10 | 1037 | RTA00000121A.f.8.1 |
| M00001497A:G02 | 2623 | 89.F3.sp6:130729.Seq |
| M00001497A:G02 | 2623 | RTA00000183AF.a.6.1 |
| M00001500A:E11 | 2623 | RTA00000183AF.b.14.1 |
| M00001500A:E11 | 2623 | 89.A4.sp6:130670.Seq |
| M00001501D:C02 | 9685 | RTA00000183AF.c.11.1.Seq_THC109544 |
| M00001501D:C02 | 9685 | RTA00000183AF.c.11.1 |
| M00001501D:C02 | 9685 | 89.C4.sp6:130694.Seq |
| M00001504C:H06 | 6974 | 89.F4.sp6:130730.Seq |
| M00001504C:H06 | 6974 | RTA00000183AF.d.9.1 |
| M00001504C:H06 | 6974 | RTA00000183AF.d.9.1.Seq_THC223129 |
| M00001504D:G06 | 6420 | 173.F5.SP6:134133.Seq |
| M00001504D:G06 | 6420 | 89.G4.sp6:130742.Seq |
| M00001504D:G06 | 6420 | RTA00000183AF.d.11.1.Seq_THC226443 |
| M00001504D:G06 | 6420 | RTA00000183AF.d.11.1 |
| M00001528A:C04 | 35555 | 89.B6.sp6:130684.Seq |
| M00001528A:C04 | 7337 | RTA00000123A.b.17.1 |
| M00001528A:C04 | 35555 | 184.A5.sp6:135530.Seq |
| M00001537B:G07 | 3389 | RTA00000183AF.m.19.1 |
| M00001537B:G07 | 3389 | 89.A8.sp6:130674.Seq |
| M00001541A:D02 | 3765 | 89.C8.sp6:130698.Seq |
| M00001541A:D02 | 3765 | RTA00000135A.d.1.1 |
| M00001544B:B07 | 6974 | 89.A9.sp6:130675.Seq |
| M00001544B:B07 | 6974 | RTA00000184AF.a.15.1 |
| M00001546A:G11 | 1267 | 89.D9.sp6:130711.Seq |
| M00001546A:G11 | 1267 | RTA00000125A.o.5.1 |
| M00001549B:F06 | 4193 | 89.G9.sp6:130747.Seq |
| M00001549B:F06 | 4193 | RTA00000184AF.e.13.1 |
| M00001556A:F11 | 1577 | 173.C9.SP6:134101.Seq |
| M00001556A:F11 | 1577 | 89.F11.sp6:130737.Seq |
| M00001556A:F11 | 1577 | RTA00000184AF.i.23.1 |
| M00001556B:C08 | 4386 | RTA00000184AF.j.4.1 |
| M00001556B:C08 | 4386 | 89.H11.sp6:130761.Seq |
| M00001563B:F06 | 102 | RTA00000184AF.o.5.1 |
| M00001563B:F06 | 102 | 90.B1.sp6:130871.Seq |
| M00001571C:H06 | 5749 | 90.E1.sp6:130907.Seq |
| M00001571C:H06 | 5749 | RTA00000185AF.a.19.1 |
| M00001594B:H04 | 260 | 90.D2.sp6:130896.Seq |
| M00001594B:H04 | 260 | RTA00000185AR.i.12.2 |
| M00001597C:H02 | 4837 | 90.E2.sp6:130908.Seq |
| M00001597C:H02 | 4837 | RTA00000185AR.k.3.2 |
| M00001624C:F01 | 4309 | 90.C4.sp6:130886.Seq |
| M00001624C:F01 | 4309 | RTA00000186AF.e.22.1 |
| M00001679A:A06 | 6660 | 90.F6.sp6:130924.Seq |
| M00001676A:A06 | 6660 | 122.B5.sp6:132089.Seq |
| M00001679A:A06 | 6660 | RTA00000187AF.h.15.1 |
| M00003759B:B09 | 697 | 90.G8.sp6:130938.Seq |
| M00003759B:B09 | 697 | RTA00000188AF.d.6.1 |
| M00003759B:B09 | 697 | RTA00000188AF.d.6.1.Seq_THC178884 |
| M00003844C:B11 | 6539 | 176.D9.sp6:134556.Seq |
| M00003844C:B11 | 6539 | RTA00000189Af.d.22.1 |
| M00003844C:B11 | 6539 | 90.B10.sp6:130880.Seq |
| M00003857A:G10 | 3389 | 90.A11.sp6:130869.Seq |
| M00003857A:G10 | 3389 | RTA00000189AF.g.3.1 |
| M00003914C:F05 | 3900 | 99.E1.sp6:131278.Seq |
| M00003914C:F05 | 3900 | RTA00000190AF.g.13.1 |
| M00003922A:E06 | 23255 | RTA00000190AF.j.4.1 |
| M00003922A:E06 | 23255 | 99.F1.sp6:131290.Seq |
| M00003922A:E06 | 23255 | RTA00000190AF.j.4.1.Seq_THC228776 |
| M00003983A:A05 | 9105 | 99.C3.sp6:131256.Seq |
| M00003983A:A05 | 9105 | RTA00000191AF.a.21.2 |
| M00004028D:A06 | 6124 | RTA00000191AR.e.2.3 |
| M00004028D:A06 | 6124 | 99.D3.sp6:131268.Seq |
| M00004031A:A12 | 9061 | RTA00000191AR.e.11.2 |
| M00004031A:A12 | 9061 | RTA00000191AR.e.11.3 |
| M00004087D:A01 | 6880 | RTA00000191AF.m.20.1 |
| M00004087D:A01 | 6880 | 99.A5.sp6:131234.Seq |
| M00004108A:E06 | 4937 | 99.E5.sp6:131282.Seq |
| M00004108A:E06 | 4937 | RTA00000191AF.p.21.1 |
| M00004114C:F11 | 13183 | 123.D5.sp6:132305.Seq |
| M00004114C:F11 | 13183 | RTA00000192AF.a.24.1 |
| M00004114C:F11 | 13183 | 99.G5.sp6:131306.Seq |
| M00004146C:C11 | 5257 | 99.B6.sp6:131247.Seq |
| M00004146C:C11 | 5257 | 177.F5.sp6:134768.Seq |
| M00004146C:C11 | 5257 | RTA00000192AF.f.3.1 |
| M00004146C:C11 | 5257 | RTA00000192AF.f.3.1.Seq_THC213833 |
| M00004157C:A09 | 6455 | RTA00000192AF.g.23.1 |
| M00004157C:A09 | 6455 | 99.D6.sp6:131271.Seq |
| M00004157C:A09 | 6455 | 123.E7.sp6:132319.Seq |
| M00004172C:D08 | 11494 | RTA00000192AF.j.6.1 |
| M00004172C:D08 | 11494 | 99.G6.sp6:131307.Seq |
| M00004172C:D08 | 11494 | 177.E6.sp6:134757.Seq |
| M00004229B:F08 | 6455 | RTA00000193AF.b.9.1 |
| M00004229B:F08 | 6455 | 99.C8.sp6:131261.Seq |
| M00001466A:E07 | 4275 | RTA00000120A.j.14.1 |
| M00001531A:H11 | | 89.F6.sp6:130732.Seq |
| M00001531A:H11 | | RTA00000123A.g.19.1 |
| M00001551A:B10 | 6268 | 79.G9.sp6:130096.Seq |
| M00001551A:B10 | 6268 | 184.C12.sp6:135561.Seq |
| M00001551A:B10 | 6268 | RTA00000126A.o.23.1 |
| M00001552A:B12 | 307 | RTA00000136A.o.4.2 |
| M00001552A:B12 | 307 | 79.C7.sp6:130046.Seq |
| M00001556A:H01 | 15855 | RTA00000184AF.j.1.1 |
| M00001586C:C05 | 4623 | RTA00000185AF.f.4.1 |
| M00001604A:B10 | 1399 | 79.G8.sp6:130095.Seq |
| M00001604A:B10 | 1399 | RTA00000129A.o.10.1 |
| M00003879B:C11 | 5345 | RTA00000189AF.l.19.1 |
| M00003879B:C11 | 5345 | 90.B12.sp6:130882.Seq |
| M00001358C:C06 | | RTA00000177AF.o.4.3 |
| M00001388D:G05 | 5832 | 80.F6.sp6:130273.Seq |
| M00001388D:G05 | 5832 | RTA00000178AF.o.23.1 |
| M00001394A:F01 | 6583 | RTA00000179AF.d.13.1 |
| M00001394A:F01 | 6583 | 172.B8.sp6:133896.Seq |
| M00001394A:F01 | 6583 | 80.H6.sp6:130297.Seq |
| M00001429A:H04 | 2797 | RTA00000180AF.i.19.1 |
| M00001447A:G03 | 10717 | RTA00000181AF.d.10.1 |
| M00001448D:C09 | 8 | 80.H10.sp6:130301.Seq |
| M00001448D:C09 | 8 | RTA00000181AF.e.17.1 |
| M00001448D:C09 | 8 | 100.B11.sp6:131444.Seq |
| M00001454D:G03 | 689 | RTA00000181AR.l.22.1 |
| M00003975A:G11 | 12439 | RTA00000190AF.o.24.1 |
| M00003978B:G05 | 5693 | RTA00000190AF.p.17.2.Seq_THC173318 |
| M00003978B:G05 | 5693 | RTA00000190AF.p.17.2 |
| M00004059A:D06 | 5417 | RTA00000191AF.h.19.1 |
| M00004068B:A01 | 3706 | 99.C4.sp6:131257.Seq |
| M00004068B:A01 | 3706 | RTA00000191AF.i.17.2 |
| M00004205D:F06 | | 99.E7.sp6:131284.Seq |
| M00004205D:F06 | | 177.G7.sp6:134782.Seq |
| M00004205D:F06 | | RTA00000192AF.o.11.1 |
| M00004212B:C07 | 2379 | RTA00000192AF.p.8.1 |
| M00004223A:G10 | 16918 | RTA00000193AF.a.16.1 |
| M00004223B:D09 | 7899 | RTA00000193AF.a.17.1 |
| M00004249D:G12 | | RTA00000193AF.c.22.1 |
| M00004251C:G07 | | RTA00000193AF.d.2.1 |
| M00004372A:A03 | 2030 | RTA00000193AF.m.20.1 |
| M00001340B:A06 | 17062 | 80.A1.sp6:130208.Seq |
| M00001340B:A06 | 17062 | RTA00000177AF.b.8.4 |
| M00001340D:F10 | 11589 | 80.B1.sp6:130220.Seq |
| M00001340D:F10 | 11589 | RTA00000177AF.b.17.4 |
| M00001341A:E12 | 4443 | 80.C1.sp6:130232.Seq |
| M00001341A:E12 | 4443 | RTA00000177AF.b.20.4 |
| M00001342B:E06 | 39805 | 80.D1.sp6:130244.Seq |
| M00001342B:E06 | 39805 | RTA00000177AF.c.21.3 |
| M00001346A:F09 | 5007 | RTA00000177AF.g.2.1 |
| M00001346A:F09 | 5007 | 80.H1.sp6:130292.Seq |
| M00001346D:G06 | 5779 | RTA00000177AF.g.14.3 |
| M00001346D:G06 | 5779 | RTA00000177AF.g.14.1 |
| M00001348B:B04 | 16927 | 80.E2.sp6:130257.Seq |
| M00001348B:B04 | 16927 | RTA00000177AF.h.9.3 |
| M00001348B:G06 | 16985 | RTA00000177AF.h.10.1 |
| M00001348B:G06 | 16985 | 80.F2.sp6:130269.Seq |
| M00001349B:B08 | 3584 | RTA00000177AF.h.20.1 |
| M00001349B:B08 | 3584 | 80.G2.sp6:130281.Seq |
| M00001350A:H01 | 7187 | 100.C2.sp6:131447.Seq |
| M00001350A:H01 | 7187 | 80.A3.sp6:130210.Seq |
| M00001350A:H01 | 7187 | RTA00000177AF.i.8.2 |
| M00001352A:E02 | 16245 | RTA00000177AF.k.9.3 |
| M00001352A:E02 | 16245 | 172.D2.sp6:133914.Seq |
| M00001352A:E02 | 16245 | 80.D3.sp6:130246.Seq |
| M00001355B:G10 | 14391 | RTA00000177AF.m.17.3 |
| M00001355B:G10 | 14391 | 80.G3.sp6:130282.Seq |
| M00001355B:G10 | 14391 | 172.H3.sp6:133963.Seq |
| M00001355B:G10 | 14391 | 100.E3.sp6:131472.Seq |
| M00001361D:F08 | 2379 | 80.C4.sp6:130235.Seq |
| M00001361D:F08 | 2379 | RTA00000178AF.a.6.1 |
| M00001365C:C10 | 40132 | RTA00000178AF.c.7.1 |
| M00001365C:C10 | 40132 | 80.F4.sp6:130271.Seq |
| M00001368D:E03 | | 80.G4.sp6:130283.Seq |
| M00001368D:E03 | | RTA00000178AF.d.20.1 |
| M00001370A:C09 | 6867 | 80.H4.sp6:130295.Seq |
| M00001370A:C09 | 6867 | RTA00000178AF.e.12.1 |
| M00001371C:E09 | 7172 | 100.A5.sp6:131426.Seq |
| M00001371C:E09 | 7172 | RTA00000178AF.f.9.1 |
| M00001371C:E09 | 7172 | 80.A5.sp6:130212.Seq |
| M00001378B:B02 | 39833 | 80.C5.sp6:130236.Seq |
| M00001378B:B02 | 39833 | RTA00000178AF.i.23.1 |
| M00001379A:A05 | 1334 | 80.D5.sp6:130248.Seq |
| M00001379A:A05 | 1334 | RTA00000178AF.j.7.1 |
| M00001380D:B09 | 39886 | RTA00000178AF.j.24.1 |
| M00001380D:B09 | 39886 | 80.E5.sp6:130260.Seq |
| M00001381D:E06 | | 80.F5.sp6:130272.Seq |
| M00001381D:E06 | | RTA00000178AF.k.16.1 |
| M00001382C:A02 | 22979 | 80.G5.sp6:130284.Seq |
| M00001382C:A02 | 22979 | RTA00000178AF.k.22.1 |
| M00001384B:A11 | | 80.B6.sp6:130225.Seq |
| M00001384B:A11 | | RTA00000178AF.m.13.1 |
| M00001386C:B12 | 5178 | 80.C6.sp6:130237.Seq |
| M00001386C:B12 | 5178 | RTA00000178AF.n.10.1 |
| M00001387B:G03 | 7587 | 80.E6.sp6:130261.Seq |
| M00001387B:G03 | 7587 | RTA00000178AF.n.24.1 |
| M00001389A:C08 | 16269 | RTA00000178AF.p.1.1 |
| M00001389A:C08 | 16269 | 80.G6.sp6:130285.Seq |
| M00001396A:C03 | 4009 | 172.D8.sp6:133920.Seq |
| M00001396A:C03 | 4009 | 80.A7.sp6:130214.Seq |
| M00001396A:C03 | 4009 | RTA00000179AF.e.20.1 |
| M00001400B:H06 | | 172.B9.sp6:133897.Seq |
| M00001400B:H06 | | 80.B7.sp6:130226.Seq |
| M00001400B:H06 | | RTA00000179AF.j.13.1 |
| M00001400B:H06 | | RTA00000179AF.j.13.1.Seq_THC105720 |
| M00001402A:E08 | 39563 | 80.C7.sp6:130238.Seq |
| M00001402A:E08 | 39563 | RTA00000179AF.k.20.1 |
| M00001407B:D11 | 5556 | RTA00000179AF.n.10.1 |
| M00001407B:D11 | 5556 | 80.D7.sp6:130250.Seq |
| M00001410A:D07 | 7005 | 180.H5.sp6:136003.Seq |
| M00001410A:D07 | 7005 | RTA00000179AF.o.22.1 |
| M00001410A:D07 | 7005 | 80.F7.sp6:130274.Seq |
| M00001414A:B01 | | RTA00000180AF.a.9.1 |
| M00001414A:B01 | | 80.H7.sp6:130298.Seq |
| M00001414C:A07 | | 80.A8.sp6:130215.Seq |
| M00001414C:A07 | | RTA00000180AF.a.11.1 |
| M00001416A:H01 | 7674 | 79.C1.sp6:130040.Seq |
| M00001416A:H01 | 7674 | RTA00000118A.g.9.1 |
| M00001417A:E02 | 36393 | RTA00000180AF.c.2.1 |
| M00001417A:E02 | 36393 | 80.D8.sp6:130251.Seq |
| M00001423B:E07 | 15066 | RTA00000180AF.e.24.1 |
| M00001423B:E07 | 15066 | 80.H8.sp6:130299.Seq |
| M00001424B:G09 | 10470 | 80.A9.sp6:130216.Seq |
| M00001424B:G09 | 10470 | RTA00000180AF.f.18.1 |
| M00001425B:H08 | 22195 | RTA00000180AF.g.7.1 |
| M00001425B:H08 | 22195 | 80.B9.sp6:130228.Seq |
| M00001426B:D12 | | RTA00000180AF.g.22.1 |
| M00001426B:D12 | | 80.C9.sp6:130240.Seq |
| M00001426D:C08 | 4261 | 80.D9.sp6:130252.Seq |
| M00001426D:C08 | 4261 | RTA00000180AF.h.5.1 |
| M00001428A:H10 | 84182 | 100.G9.sp6:131502.Seq |
| M00001428A:H10 | 84182 | RTA00000180AF.h.19.1 |
| M00001428A:H10 | 84182 | 80.E9.sp6:130264.Seq |
| M00001449A:A12 | 5857 | 80.B11.sp6:130230.Seq |
| M00001449A:A12 | 5857 | RTA00000118A.g.14.1 |
| M00001449A:B12 | 41633 | 80.C11.sp6:130242.Seq |
| M00001449A:B12 | 41633 | RTA00000118A.g.16.1 |
| M00001449A:G10 | 36535 | RTA00000181AF.f.5.1 |
| M00001449A:G10 | 36535 | 80.D11.sp6:130254.Seq |
| M00001449A:G10 | 36535 | 100.D11.sp6:131468.Seq |
| M00001449C:D06 | 86110 | RTA00000181AF.f.12.1 |
| M00001449C:D06 | 86110 | 80.E11.sp6:130266.Seq |
| M00001450A:A02 | 39304 | RTA00000118A.j.21.1.Seq_THC151859 |
| M00001450A:A02 | 39304 | RTA00000118A.j.21.1 |
| M00001450A:A02 | 39304 | 79.F1.sp6:130076.Seq |
| M00001450A:A02 | 39304 | 180.G9.sp6:135995.Seq |
| M00001450A:A11 | 32663 | 80.F11.sp6:130278.Seq |
| M00001450A:A11 | 32663 | RTA00000118A.l.8.1 |
| M00001450A:B12 | 82498 | 100.F11.sp6:131492.Seq |
| M00001450A:B12 | 82498 | RTA00000118A.m.10.1 |
| M00001450A:B12 | 82498 | 79.G1.sp6:130088.Seq |
| M00001450A:D08 | 27250 | 80.G11.sp6:130290.Seq |
| M00001450A:D08 | 27250 | 180.B10.sp6:135936.Seq |
| M00001450A:D08 | 27250 | RTA00000181AF.g.10.1 |
| M00001452A:B04 | 84328 | RTA00000118A.p.10.1 |
| M00001452A:B04 | 84328 | 79.A2.sp6:130017.Seq |
| M00001452A:B12 | 86859 | RTA00000118A.p.8.1 |
| M00001452A:B12 | 86859 | 79.B2.sp6:130029.Seq |
| M00001452A:F05 | 85064 | RTA00000131A.m.23.1 |
| M00001452A:F05 | 85064 | 79.D2.sp6:130053.Seq |
| M00001452C:B06 | 16970 | 80.H11.sp6:130302.Seq |
| M00001452C:B06 | 16970 | 100.C12.sp6:131457.Seq |
| M00001452C:B06 | 16970 | RTA00000181AR.i.18.2 |
| M00001453A:E11 | 16130 | 80.A12.sp6:130219.Seq |
| M00001453A:E11 | 16130 | 100.D12.sp6:131469.Seq |
| M00001453A:E11 | 16130 | RTA00000119A.c.13.1 |
| M00001453C:F06 | 16653 | 80.B12.sp6:130231.Seq |
| M00001453C:F06 | 16653 | RTA00000181AF.k.5.3 |
| M00001454A:A09 | 83103 | RTA00000119A.e.24.2 |
| M00001454A:A09 | 83103 | 79.G2.sp6:130089.Seq |
| M00001454B:C12 | 7005 | 121.D1.sp6:131917.Seq |
| M00001454B:C12 | 7005 | RTA00000181AF.k.24.1 |
| M00001454B:C12 | 7005 | 80.C12.sp6:130243.Seq |
| M00001455B:E12 | 13072 | 80.F12.sp6:130279.Seq |
| M00001455B:E12 | 13072 | RTA00000181AR.m.5.2 |
| M00001460A:F06 | 2448 | 89.A1.sp6:130667.Seq |
| M00001460A:F06 | 2448 | RTA00000119A.j.21.1 |
| M00001461A:D06 | 1531 | 89.C1.sp6:130691.Seq |
| M00001461A:D06 | 1531 | RTA00000119A.o.3.1 |
| M00001465A:B11 | 10145 | 79.F3.sp6:130078.Seq |
| M00001465A:B11 | 10145 | RTA00000120A.g.12.1 |
| M00001467A:B07 | 38759 | 89.F1.sp6:130727.Seq |
| M00001467A:B07 | 38759 | RTA00000120A.m.12.3 |
| M00001467A:D04 | 39508 | RTA00000120A.o.2.1 |
| M00001467A:D04 | 39508 | 89.G1.sp6:130739.Seq |
| M00001467A:E10 | 39442 | 89.A2.sp6:130668.Seq |
| M00001467A:E10 | 39442 | RTA00000120A.o.21.1 |
| M00001468A:F05 | 7589 | RTA00000120A.p.23.1 |
| M00001468A:F05 | 7589 | 89.B2.sp6:130680.Seq |
| M00001469A:A01 | | RTA00000121A.c.10.1 |
| M00001469A:A01 | | 89.C2.sp6:130692.Seq |
| M00001469A:C10 | 12081 | 89.D2.sp6:130704.Seq |
| M00001469A:C10 | 12081 | RTA00000133A.d.14.2 |
| M00001469A:H12 | 19105 | 89.E2.sp6:130716.Seq |
| M00001469A:H12 | 19105 | RTA00000133A.e.15.1 |
| M00001470A:C04 | 39425 | 89.G2.sp6:130740.Seq |
| M00001470A:C04 | 39425 | RTA00000133A.f.1.1 |
| M00001471A:B01 | 39478 | 89.H2.sp6:130752.Seq |
| M00001471A:B01 | 39478 | RTA00000133A.i.5.1 |
| M00001487B:H06 | | RTA00000182AF.l.15.1 |
| M00001487B:H06 | | 89.B3.sp6:130681.Seq |
| M00001488B:F12 | | RTA00000182AF.l.20.1 |
| M00001488B:F12 | | 89.C3.sp6:130693.Seq |
| M00001494D:F06 | 7206 | RTA00000182AF.o.15.1 |
| M00001494D:F06 | 7206 | 89.E3.sp6:130717.Seq |
| M00001499B:A11 | 10539 | RTA00000183AF.a.24.1 |
| M00001499B:A11 | 10539 | 89.G3.sp6:130741.Seq |
| M00001499B:A11 | 10539 | 173.B5.SP6:134085.Seq |
| M00001500A:C05 | 5336 | RTA00000183AF.b.13.1 |
| M00001500A:C05 | 5336 | 89.H3.sp6:130753.Seq |
| M00001504A:E01 | | RTA00000183AF.c.24.1 |
| M00001504A:E01 | | 89.D4.sp6:130706.Seq |
| M00001504A:E01 | | RTA00000183AF.c.24.1.Seq_THC125912 |
| M00001504C:A07 | 10185 | RTA00000183AF.d.5.1 |
| M00001504C:A07 | 10185 | 89.E4.sp6:130718.Seq |
| M00001505C:C05 | | 89.H4.sp6:130754.Seq |
| M00001505C:C05 | | RTA00000183AFe.1.1 |
| M00001506D:A09 | | 89.A5.sp6:130671.Seq |
| M00001506D:A09 | | RTA00000183AF.e.23.1 |
| M00001506D:A09 | | 121.G6.sp6:131958.Seq |
| M00001507A:H05 | 39168 | RTA00000121A.l.10.1 |
| M00001507A:H05 | 39168 | 89.B5.sp6:130683.Seq |
| M00001535A:F10 | 39423 | 79.C5.sp6:130044.Seq |
| M00001535A:F10 | 39423 | RTA00000134A.k.22.1 |
| M00001541A:H03 | 39174 | 79.E5.sp6:130068.Seq |
| M00001541A:H03 | 39174 | RTA00000124A.n.13.1 |
| M00001544A:G02 | 19829 | 79.H5.sp6:130104.Seq |
| M00001544A:G02 | 19829 | RTA00000125A.h.24.4 |
| M00001545A:D08 | 13864 | RTA00000125A.m.9.1 |
| M00001545A:D08 | 13864 | 79.B6.sp6:130033.Seq |
| M00001551A:F05 | 39180 | RTA00000126A.n.8.2 |
| M00001551A:F05 | 39180 | 79.A7.sp6:130022.Seq |
| M00001552A:D11 | 39458 | RTA00000126A.p.15.2 |
| M00001552A:D11 | 39458 | 79.D7.sp6:130058.Seq |
| M00001557A:F03 | 39490 | RTA00000128A.b.4.1 |
| M00001511A:H06 | 39412 | RTA00000133A.k.17.1 |
| M00001511A:H06 | 39412 | 89.C5.sp6:130695.Seq |
| M00001512A:A09 | 39186 | 89.D5.sp6:130707.Seq |
| M00001512A:A09 | 39186 | RTA00000121A.p.15.1 |
| M00001512D:G09 | 3956 | 89.E5.sp6:130719.Seq |
| M00001512D:G09 | 3956 | 173.H5.SP6:134157.Seq |
| M00001512D:G09 | 3956 | RTA00000183AF.g.3.1 |
| M00001513B:G03 | | RTA00000183AF.g.9.1 |
| M00001513B:G03 | | 89.F5.sp6:130731.Seq |
| M00001513B:G03 | | RTA00000183AF.g.9.1.Seq_THC198280 |
| M00001513C:E08 | 14364 | RTA00000183AF.g.12.1 |
| M00001513C:E08 | 14364 | 89.G5.sp6:130743.Seq |
| M00001514C:D11 | 40044 | RTA00000183AF.g.22.1 |
| M00001514C:D11 | 40044 | RTA00000183AF.g.22.1.Seq_THC232899 |
| M00001514C:D11 | 40044 | 89.H5.sp6:130755.Seq |
| M00001518C:B11 | 8952 | 89.A6.sp6:130672.Seq |
| M00001518C:B11 | 8952 | RTA00000183AF.h.15.1 |
| M00001528B:H04 | 8358 | 89.D6.sp6:130708.Seq |
| M00001528B:H04 | 8358 | RTA00000183AF.i.5.1 |
| M00001531A:D01 | 38085 | RTA00000123A.e.15.1 |
| M00001531A:D01 | 38085 | 89.E6.sp6:130720.Seq |
| M00001534A:C04 | 16921 | RTA00000183AF.k.6.1 |
| M00001534A:C04 | 16921 | 89.H6.sp6:130756.Seq |
| M00001534A:D09 | 5097 | RTA00000134A.k.1.1 |
| M00001534A:D09 | 5097 | RTA00000134A.k.1.1.Seq_THC215869 |
| M00001534C:A01 | 4119 | RTA00000183AF.k.16.1 |
| M00001534C:A01 | 4119 | 89.C7.sp6:130697.Seq |
| M00001535A:C06 | 20212 | 89.E7.sp6:130721.Seq |
| M00001535A:C06 | 20212 | RTA00000134A.l.22.1.Seq_THC128232 |
| M00001535A:C06 | 20212 | RTA00000134A.l.22.1 |
| M00001536A:B07 | 2696 | RTA00000134A.m.13.1 |
| M00001536A:B07 | 2696 | 89.F7.sp6:130733.Seq |
| M00001537A:F12 | 39420 | 89.H7.sp6:130757.Seq |
| M00001537A:F12 | 39420 | RTA00000134A.o.23.1 |
| M00001540A:D06 | 8286 | 89.B8.sp6:130686.Seq |
| M00001540A:D06 | 8286 | RTA00000183AF.o.1.1 |
| M00001542A:E06 | 39453 | 89.E8.sp6:130722.Seq |
| M00001542A:E06 | 39453 | RTA00000135A.g.11.1 |
| M00001544A:E06 | | RTA00000184AF.a.8.1 |
| M00001544A:E06 | | 173.G7.SP6:134147.Seq |
| M00001544A:E06 | | 89.H8.sp6:130758.Seq |
| M00001545A:B02 | | 89.B9.sp6:130687.Seq |
| M00001545A:B02 | | RTA00000135A.l.2.2 |
| M00001548A:E10 | 5892 | 89.E9.sp6:130723.Seq |
| M00001548A:E10 | 5892 | RTA00000184AF.d.11.1 |
| M00001548A:E10 | 5892 | RTA00000184AF.d.11.1.Seq_THC161896 |
| M00001549C:E06 | 16347 | 89.H9.sp6:130759.Seq |
| M00001549C:E06 | 16347 | RTA00000184AF.e.15.1 |
| M00001550A:A03 | 7239 | 89.A10.sp6:130676.Seq |
| M00001550A:A03 | 7239 | RTA00000126A.m.4.2 |
| M00001550A:G01 | 5175 | RTA00000184AF.f.3.1 |
| M00001550A:G01 | 5175 | 89.B10.sp6:130688.Seq |
| M00001551A:G06 | 22390 | RTA00000136A.j.13.1 |
| M00001551A:G06 | 22390 | 89.C10.sp6:130700.Seq |
| M00001551C:G09 | 3266 | RTA00000184AR.g.1.1 |
| M00001551C:G09 | 3266 | 89.D10.sp6:130712.Seq |
| M00001553A:H06 | 8298 | RTA00000127A.d.19.1 |
| M00001553A:H06 | 8298 | 89.G10.sp6:130748.Seq |
| M00001553B:F12 | 4573 | 89.H10.sp6:130760.Seq |
| M00001553B:F12 | 4573 | RTA00000184AF.h.9.1 |
| M00001555A:B02 | 39539 | RTA00000127A.i.21.1 |
| M00001555A:B02 | 39539 | 89.B11.sp6:130689.Seq |
| M00001555A:C01 | 39195 | 89.C11.sp6:130701.Seq |
| M00001555A:C01 | 39195 | RTA00000137A.c.16.1 |
| M00001555D:G10 | 4561 | RTA00000184AF.i.21.1 |
| M00001555D:G10 | 4561 | 89.D11.sp6:130713.Seq |
| M00001556A:C09 | 9244 | 89.E11.sp6:130725.Seq |
| M00001556A:C09 | 9244 | RTA00000127A.l.3.1 |
| M00001556B:G02 | 11294 | RTA00000184AF.j.6.1 |
| M00001556B:G02 | 11294 | 89.A12.sp6:130678.Seq |
| M00001557B:H10 | 5192 | 173.E9.SP6:134125.Seq |
| M00001557B:H10 | 5192 | RTA00000184AF.k.2.1 |
| M00001557B:H10 | 5192 | 89.D12.sp6:130714.Seq |
| M00001557D:D09 | 8761 | RTA00000184AF.k.12.1 |
| M00001557D:D09 | 8761 | 89.E12.sp6:130726.Seq |
| M00001558B:H11 | 7514 | RTA00000184AF.k.21.1 |
| M00001558B:H11 | 7514 | 89.G12.sp6:130750.Seq |
| M00001559B:F01 | | 89.H12.sp6:130762.Seq |
| M00001559B:F01 | | RTA00000184AF.l.11.1 |
| M00001560D:F10 | 6558 | 90.A1.sp6:130859.Seq |
| M00001560D:F10 | 6558 | RTA00000184AF.m.21.1 |
| M00001566B:D11 | | RTA00000184AF.p.3.1 |
| M00001566B:D11 | | 90.D1.sp6:130895.Seq |
| M00001583D:A10 | 6293 | RTA00000185AF.e.11.1 |
| M00001583D:A10 | 6293 | 90.A2.sp6:130860.Seq |
| M00001590B:F03 | | RTA00000185AF.g.11.1 |
| M00001590B:F03 | | 90.C2.sp6:130884.Seq |
| M00001597D:C05 | 10470 | RTA00000185AF.k.6.1 |
| M00001597D:C05 | 10470 | 90.F2.sp6:130920.Seq |
| M00001598A:G03 | 16999 | 90.G2.sp6:130932.Seq |
| M00001598A:G03 | 16999 | RTA00000185AF.k.9.1 |
| M00001601A:D08 | 22794 | RTA00000138A.b.5.1 |
| M00001601A:D08 | 22794 | 90.H2.sp6:130944.Seq |
| M00001607A:E11 | 11465 | RTA00000185AF.m.19.1 |
| M00001607A:E11 | 11465 | 90.A3.sp6:130861.Seq |
| M00001608A:B03 | 7802 | RTA00000185AF.n.5.1 |
| M00001608A:B03 | 7802 | 90.B3.sp6:130873.Seq |
| M00001608B:E03 | 22155 | RTA00000185AF.n.9.1 |
| M00001608B:E03 | 22155 | 90.C3.sp6:130885.Seq |
| M00001608D:A11 | | RTA00000185AF.n.12.1 |
| M00001608D:A11 | | 90.D3.sp6:130897.Seq |
| M00001614C:F10 | 13157 | RTA00000186AF.a.6.1 |
| M00001614C:F10 | 13157 | 90.E3.sp6:130909.Seq |
| M00001617C:E02 | 17004 | RTA00000186AF.b.21.1 |
| M00001617C:E02 | 17004 | 90.F3.sp6:130921.Seq |
| M00001619C:F12 | 40314 | 90.G3.sp6:130933.Seq |
| M00001619C:F12 | 40314 | RTA00000186AF.c.15.1 |
| M00001621C:C08 | 40044 | RTA00000186AF.d.1.1 |
| M00001621C:C08 | 40044 | RTA00000186AF.d.1.1.Seq_THC232899 |
| M00001621C:C08 | 40044 | 90.H3.sp6:130945.Seq |
| M00001621C:C08 | 40044 | 122.E1.sp6:132121.Seq |
| M00001623D:F10 | 13913 | RTA00000186AF.e.6.1 |
| M00001623D:F10 | 13913 | 90.A4.sp6:130862.Seq |
| M00001632D:H07 | | RTA00000186AF.h.14.1.Seq_THC112525 |
| M00001632D:H07 | | RTA00000186AF.h.14.1 |
| M00001632D:H07 | | 90.E4.sp6:130910.Seq |
| M00001632D:H07 | | 176.A3.sp6:134514.Seq |
| M00001644C:B07 | 39171 | RTA00000186AF.l.7.1 |
| M00001644C:B07 | 39171 | 90.F4.sp6:130922.Seq |
| M00001644C:B07 | 39171 | 217.A12.sp6:139369.Seq |
| M00001645A:C12 | 19267 | RTA00000186AF.l.12.1.Seq_THC178183 |
| M00001645A:C12 | 19267 | 176.G3.sp6:134586.Seq |
| M00001645A:C12 | 19267 | RTA00000186AF.l.12.1 |
| M00001645A:C12 | 19267 | 90.G4.sp6:130934.Seq |
| M00001648C:A01 | 4665 | 90.H4.sp6:130946.Seq |
| M00001648C:A01 | 4665 | RTA00000186AF.m.3.1 |
| M00001657D:C03 | 23201 | RTA00000187AF.a.14.1 |
| M00001657D:C03 | 23201 | 90.B5.sp6:130875.Seq |
| M00001657D:F08 | 76760 | 90.C5.sp6:130887.Seq |
| M00001657D:F08 | 76760 | RTA00000187AF.a.15.1 |
| M00001662C:A09 | 23218 | RTA00000187AR.c.5.2 |
| M00001662C:A09 | 23218 | 90.D5.sp6:130899.Seq |
| M00001663A:E04 | 35702 | 90.E5.sp6:130911.Seq |
| M00001663A:E04 | 35702 | RTA00000187AR.c.15.2 |
| M00001669B:F02 | 6468 | 90.F5.sp6:130923.Seq |
| M00001669B:F02 | 6468 | RTA00000187AF.d.15.1 |
| M00001670C:H02 | 14367 | 90.G5.sp6:130935.Seq |
| M00001670C:H02 | 14367 | RTA00000187AF.e.8.1 |
| M00001673C:H02 | 7015 | 90.H5.sp6:130947.Seq |
| M00001673C:H02 | 7015 | RTA00000187AF.f.18.1 |
| M00001675A:C09 | 8773 | RTA00000187AF.f.24.1 |
| M00001675A:C09 | 8773 | 90.A6.sp6:130864.Seq |
| M00001675A:C09 | 8773 | RTA00000187AF.f.24.1.Seq_THC220002 |
| M00001676B:F05 | 11460 | RTA00000187AF.g.12.1 |
| M00001676B:F05 | 11460 | 90.B6.sp6:130876.Seq |
| M00001676B:F05 | 11460 | 219.F2.sp6:139035.Seq |
| M00001677D:A07 | 7570 | 90.D6.sp6:130900.Seq |
| M00001677D:A07 | 7570 | RTA00000187AF.g.24.1 |
| M00001677D:A07 | 7570 | RTA00000187AF.g.24.1.Seq_THC168636 |
| M00001678D:F12 | 4416 | 90.E6.sp6:130912.Seq |
| M00001678D:F12 | 4416 | RTA00000187AF.h.13.1 |
| M00001679A:F10 | 26875 | RTA00000187AF.i.1.1 |
| M00001679A:F10 | 26875 | 90.A7.sp6:130865.Seq |
| M00001679B:F01 | 6298 | 90.B7.sp6:130877.Seq |
| M00001679B:F01 | 6298 | RTA00000187AR.i.10.2 |
| M00001680D:F08 | 10539 | 90.F7.sp6:130925.Seq |
| M00001680D:F08 | 10539 | 219.F6.sp6:139039.Seq |
| M00001680D:F08 | 10539 | RTA00000187AF.l.7.1 |
| M00001682C:B12 | 17055 | 90.G7.sp6:130937.Seq |
| M00001682C:B12 | 17055 | RTA00000187AF.m.3.1 |
| M00001682C:B12 | 17055 | 176.D6.sp6:134553.Seq |
| M00001688C:F09 | 5382 | 90.A8.sp6:130866.Seq |
| M00001688C:F09 | 5382 | RTA00000187AF.m.23.2 |
| M00001693C:G01 | 4393 | RTA00000187AF.n.17.1 |
| M00001693C:G01 | 4393 | 90.B8.sp6:130878.Seq |
| M00001716D:H05 | 67252 | RTA00000187AF.o.6.1 |
| M00001716D:H05 | 67252 | 90.C8.sp6:130890.Seq |
| M00003741D:C09 | 40108 | 90.D8.sp6:130902.Seq |
| M00003741D:C09 | 40108 | RTA00000187AF.o.24.1 |
| M00003747D:C05 | 11476 | RTA00000187AF.p.19.1 |
| M00003747D:C05 | 11476 | 90.E8.sp6:130914.Seq |
| M00003747D:C05 | 11476 | RTA00000187AF.p.19.1.Seq_THC108482 |
| M00003747D:C05 | 11476 | 219.H8.sp6:139065.Seq |
| M00003754C:E09 | | 90.F8.sp6:130926.Seq |
| M00003754C:E09 | | RTA00000188AF.b.12.1 |
| M00003761D:A09 | | RTA00000188AF.d.11.1 |
| M00003761D:A09 | | 90.H8.sp6:130950.Seq |
| M00003761D:A09 | | RTA00000188AF.d.11.1.Seq_THC212094 |
| M00003762C:B08 | 17076 | RTA00000188AF.d.21.1.Seq_THC208760 |
| M00003762C:B08 | 17076 | 90.A9.sp6:130867.Seq |
| M00003762C:B08 | 17076 | RTA00000188AF.d.21.1 |
| M00003763A:F06 | 3108 | RTA00000188AF.d.24.1 |
| M00003763A:F06 | 3108 | 90.B9.sp6:130879.Seq |
| M00003774C:A03 | 67907 | RTA00000188AF.g.11.1.Seq_THC123222 |
| M00003774C:A03 | 67907 | RTA00000188AF.g.11.1 |
| M00003774C:A03 | 67907 | 90.C9.sp6:130891.Seq |
| M00003784D:D12 | | RTA00000188AF.i.8.1 |
| M00003784D:D12 | | 90.D9.sp6:130903.Seq |
| M00003839A:D08 | 7798 | RTA00000189AF.c.18.1 |
| M00003839A:D08 | 7798 | 90.A10.sp6:130868.Seq |
| M00003851B:D08 | | 90.D10.sp6:130904.Seq |
| M00003851B:D08 | | RTA00000189AF.f.7.1 |
| M00003851B:D10 | 13595 | 90.E10.sp6:130916.Seq |
| M00003851B:D10 | 13595 | RTA00000189AF.f.8.1 |
| M00003853A:D04 | 5619 | 90.F10.sp6:130928.Seq |
| M00003853A:D04 | 5619 | RTA00000189AF.f.17.1 |
| M00003853A:F12 | 10515 | 90.G10.sp6:130940.Seq |
| M00003853A:F12 | 10515 | RTA00000189AF.f.18.1 |
| M00003856B:C02 | 4622 | 90.H10.sp6:130952.Seq |
| M00003856B:C02 | 4622 | RTA00000189AF.g.1.1 |
| M00003857A:H03 | 4718 | 90.B11.sp6:130881.Seq |
| M00003857A:H03 | 4718 | RTA00000189AF.g.5.1.Seq_THC196102 |
| M00003857A:H03 | 4718 | RTA00000189AF.g.5.1 |
| M00003867A:D10 | | 90.C11.sp6:130893.Seq |
| M00003867A:D10 | | RTA00000189AF.h.17.1 |
| M00003871C:E02 | 4573 | RTA00000189AF.j.12.1 |
| M00003875C:G07 | 8479 | 90.G11.sp6:130941.Seq |
| M00003875C:G07 | 8479 | RTA00000189AF.j.22.1 |
| M00003875D:D11 | | 90.H11.sp6:130953.Seq |
| M00003875D:D11 | | RTA00000189AF.j.23.1 |
| M00003876D:E12 | 7798 | 90.A12.sp6:130870.Seq |
| M00003876D:E12 | 7798 | RTA00000189AF.k.12.1 |
| M00003906C:E10 | 9285 | 90.H12.sp6:130954.Seq |
| M00003906C:E10 | 9285 | RTA00000190AF.d.7.1 |
| M00003907D:A09 | 39809 | 99.A1.sp6:131230.Seq |
| M00003907D:A09 | 39809 | RTA00000190AF.e.3.1.Seq_THC150217 |
| M00003907D:A09 | 39809 | RTA00000190AF.e.3.1 |
| M00003907D:H04 | 16317 | 99.B1.sp6:131242.Seq |
| M00003907D:H04 | 16317 | RTA00000190AF.e.6.1 |
| M00003909D:C03 | 8672 | RTA00000190AF.f.11.1 |
| M00003909D:C03 | 8672 | 99.C1.sp6:131254.Seq |
| M00003968B:F06 | 24488 | RTA00000190AF.n.16.1 |
| M00003968B:F06 | 24488 | 99.C2.sp6:131255.Seq |
| M00003970C:B09 | 40122 | RTA00000190AF.n.23.1 |
| M00003970C:B09 | 40122 | RTA00000190AF.n.23.1.Seq_THC109227 |
| M00003970C:B09 | 40122 | 99.D2.sp6:131267.Seq |
| M00003974D:E07 | 23210 | RTA00000190AF.o.20.1 |
| M00003974D:E07 | 23210 | RTA00000190AF.o.20.1.Seq_THC207240 |
| M00003974D:E07 | 23210 | 99.E2.sp6:131279.Seq |
| M00003974D:H02 | 23358 | RTA00000190AF.o.21.1.Seq_THC207240 |
| M00003974D:H02 | 23358 | RTA00000190AF.o.21.1 |
| M00003974D:H02 | 23358 | 99.F2.sp6:131291.Seq |
| M00003981A:E10 | 3430 | 99.A3.sp6:131232.Seq |
| M00003981A:E10 | 3430 | RTA00000191AF.a.9.1 |
| M00003982C:C02 | 2433 | RTA00000191AF.a.15.2 |
| M00003982C:C02 | 2433 | 99.B3.sp6:131244.Seq |
| M00003982C:C02 | 2433 | RTA00000191AF.a.15.2.Seq_THC79498 |
| M00004028D:C05 | 40073 | RTA00000191AF.e.3.1 |
| M00004028D:C05 | 40073 | 99.E3.sp6:131280.Seq |
| M00004035C:A07 | 37285 | 99.H3.sp6:131316.Seq |
| M00004035C:A07 | 37285 | RTA00000191AF.f.11.1 |
| M00004035D:B06 | 17036 | RTA00000191AF.f.13.1 |
| M00004035D:B06 | 17036 | 99.A4.sp6:131233.Seq |
| M00004072A:C03 | | RTA00000191AF.j.9.1 |
| M00004072A:C03 | | 99.D4.sp6:131269.Seq |
| M00004081C:D10 | 15069 | 99.F4.sp6:131293.Seq |
| M00004081C:D10 | 15069 | RTA00000191AF.l.6.1 |
| M00004086D:G06 | 9285 | 99.H4.sp6:131317.Seq |
| M00004086D:G06 | 9285 | RTA00000191AF.m.18.1 |
| M00004105C:A04 | 7221 | 99.D5.sp6:131270.Seq |
| M00004105C:A04 | 7221 | RTA00000191AF.p.9.1 |
| M00004171D:B03 | 4908 | RTA00000192AF.j.2.1 |
| M00004171D:B03 | 4908 | 99.F6.sp6:131295.Seq |
| M00004185C:C03 | 11443 | RTA00000192AF.l.13.2 |
| M00004185C:C03 | 11443 | 123.A8.sp6:132272.Seq |
| M00004185C:C03 | 11443 | 99.A7.sp6:131236.Seq |
| M00004191D:B11 | | RTA00000192AF.m.12.1 |
| M00004191D:B11 | | 99.B7.sp6:131248.Seq |
| M00004191D:B11 | | 123.C8.sp6:132296.Seq |
| M00004197D:H01 | 8210 | 99.C7.sp6:131260.Seq |
| M00004197D:H01 | 8210 | 123.E8.sp6:132320.Seq |
| M00004197D:H01 | 8210 | RTA00000192AF.n.13.1 |
| M00004203B:C12 | 14311 | 99.D7.sp6:131272.Seq |
| M00004203B:C12 | 14311 | RTA00000192AF.o.2.1 |
| M00004214C:H05 | 11451 | 177.D8.sp6:134747.Seq |
| M00004214C:H05 | 11451 | RTA00000192AF.p.17.1 |
| M00004223D:E04 | 12971 | RTA00000193AF.a.20.1 |
| M00004223D:E04 | 12971 | 99.B8.sp6:131249.Seq |
| M00004269D:D06 | 4905 | 99.H8.sp6:131321.Seq |
| M00004269D:D06 | 4905 | RTA00000193AF.e.14.1 |
| M00004295D:F12 | 16921 | 99.D9.sp6:131274.Seq |
| M00004295D:F12 | 16921 | RTA00000193AF.h.15.1 |
| M00004296C:H07 | 13046 | 99.E9.sp6:131286.Seq |
| M00004296C:H07 | 13046 | RTA00000193AF.h.19.1 |
| M00004307C:A06 | 9457 | RTA00000193AF.i.14.2 |
| M00004307C:A06 | 9457 | 99.F9.sp6:131298.Seq |
| M00004307C:A06 | 9457 | 123.D11.sp6:132311.Seq |
| M00004312A:G03 | 26295 | RTA00000193AF.i.24.2 |
| M00004312A:G03 | 26295 | 99.G9.sp6:131310.Seq |
| M00004312A:G03 | 26295 | RTA00000193AF.i.24.2.Seq_THC197345 |
| M00004318C:D10 | 21847 | RTA00000193AF.j.9.1 |
| M00004318C:D10 | 21847 | 99.H9.sp6:131322.Seq |
| M00004359B:G02 | | RTA00000193AF.m.5.1.Seq_THC173318 |
| M00004359B:G02 | | RTA00000193AF.m.5.1 |
| M00004505D:F08 | | RTA00000194AF.b.19.1 |
| M00004505D:F08 | | 99.H10.sp6:131323.Seq |
| M00004692A:H08 | | 99.B11.sp6:131252.Seq |
| M00004692A:H08 | | RTA00000194AF.c.24.1 |
| M00004692A:H08 | | 377.F4.sp6:141957.Seq |
| M00005180C:G03 | | RTA00000194AF.f.4.1 |
| M00001346D:E03 | 6806 | RTA00000177AF.g.13.3 |
| M00001350A:B08 | | 80.H2.sp6:130293.Seq |
| M00001350A:B08 | | RTA00000177AF.i.6.2 |
| M00001357D:D11 | 4059 | RTA00000177AF.n.18.3.Seq_THC123051 |
| M00001357D:D11 | 4059 | RTA00000177AF.n.18.3 |
| M00001409C:D12 | 9577 | RTA00000179AF.o.17.1 |
| M00001409C:D12 | 9577 | 80.E7.sp6:130262.Seq |
| M00001418B:F03 | 9952 | RTA00000180AF.c.20.1 |
| M00001418B:F03 | 9952 | RTA00000180AF.c.20.1.Seq_THC162284 |
| M00001418B:F03 | 9952 | 80.E8.sp6:130263.Seq |
| M00001418D:B06 | 8526 | RTA00000180AF.d.1.1 |
| M00001421C:F01 | 9577 | RTA00000180AF.d.23.1 |
| M00001421C:F01 | 9577 | 80.G8.sp6:130287.Seq |
| M00001429B:A11 | 4635 | RTA00000180AF.i.20.1 |
| M00001432C:F06 | | RTA00000180AF.k.24.1 |
| M00001439C:F08 | 40054 | RTA00000180AF.p.10.1 |
| M00001442C:D07 | 16731 | RTA00000181AF.a.20.1 |
| M00001442C:D07 | 16731 | 80.C10.sp6:130241.Seq |
| M00001443B:F01 | | 80.D10.sp6:130253.Seq |
| M00001443B:F01 | | RTA00000181AF.b.7.1 |
| M00001445A:F05 | 13532 | 80.E10.sp6:130265.Seq |
| M00001445A:F05 | 13532 | RTA00000181AF.c.4.1 |
| M00001446A:F05 | 7801 | RTA00000181AF.c.21.1 |
| M00001455A:E09 | 13238 | RTA00000181AF.m.4.1 |
| M00001455A:E09 | 13238 | RTA00000181AF.m.4.1.Seq_THC140691 |
| M00001460A:F12 | 39498 | RTA00000119A.j.20.1 |
| M00001481D:A05 | 7985 | RTA00000182AR.j.2.1 |
| M00001490B:C04 | 18699 | RTA00000182AF.m.16.1 |
| M00001490B:C04 | 18699 | 89.D3.sp6:130705.Seq |
| M00001500C:E04 | 9443 | 89.B4.sp6:130682.Seq |
| M00001500C:E04 | 9443 | RTA00000183AF.c.1.1 |
| M00001532B:A06 | 3990 | 89.G6.sp6:130744.Seq |
| M00001532B:A06 | 3990 | RTA00000183AF.j.11.1 |
| M00001534A:F09 | 5321 | 89.B7.sp6:130685.Seq |
| M00001534A:F09 | 5321 | RTA00000183AF.k.8.1 |
| M00001535A:B01 | 7665 | RTA00000134A.l.19.1 |
| M00001536A:C08 | 39392 | 89.G7.sp6:130745.Seq |
| M00001536A:C08 | 39392 | RTA00000134A.m.16.1 |
| M00001541A:F07 | 22085 | RTA00000135A.e.5.2 |
| M00001542B:B01 | | RTA00000183AF.p.4.1 |
| M00001542B:B01 | | 89.F8.sp6:130734.Seq |
| M00001544A:E03 | 12170 | RTA00000125A.h.18.4 |
| M00001545A:C03 | 19255 | RTA00000135A.m.18.1 |
| M00001545A:C03 | 19255 | 184.B10.sp6:135547.Seq |
| M00001545A:C03 | 19255 | 89.C9.sp6:130699.Seq |
| M00001548A:H09 | 1058 | RTA00000126A.e.20.3.Seq_THC217534 |
| M00001548A:H09 | 1058 | RTA00000126A.e.20.3 |
| M00001548A:H09 | 1058 | 79.F6.sp6:130081.Seq |
| M00001549A:B02 | 4015 | RTA00000136A.e.12.1 |
| M00001549A:B02 | 4015 | 79.G6.sp6:130093.Seq |
| M00001549A:D08 | 10944 | RTA00000126A.h.17.2 |
| M00001552B:D04 | 5708 | RTA00000184AF.g.12.1 |
| M00001552B:D04 | 5708 | 89.E10.sp6:130724.Seq |
| M00001552D:A01 | | 89.F10.sp6:130736.Seq |
| M00001552D:A01 | | RTA00000184AF.g.22.1 |
| M00001553D:D10 | 22814 | RTA00000184AF.h.14.1 |
| M00001553D:D10 | 22814 | 89.A11.sp6:130677.Seq |
| M00001558A:H05 | | RTA00000128A.c.20.1 |
| M00001558A:H05 | | 89.F12.sp6:130738.Seq |
| M00001561A:C05 | 39486 | RTA00000128A.m.22.2 |
| M00001561A:C05 | 39486 | 79.B8.sp6:130035.Seq |
| M00001564A:B12 | 5053 | RTA00000184AF.o.12.1 |
| M00001578B:E04 | 23001 | RTA00000185AF.c.24.1 |
| M00001579D:C03 | 6539 | 90.G1.sp6:130931.Seq |
| M00001579D:C03 | 6539 | 173.A12.SP6:134080.Seq |
| M00001579D:C03 | 6539 | RTA00000185AF.d.11.1 |
| M00001582D:F05 | | RTA00000185AF.d.24.1 |
| M00001587A:B11 | 39380 | RTA00000129A.e.24.1 |
| M00001587A:B11 | 39380 | 79.E8.sp6:130071.Seq |
| M00001604A:F05 | 39391 | RTA00000138A.c.3.1 |
| M00001604A:F05 | 39391 | 79.A9.sp6:130024.Seq |
| M00001624A:B06 | 3277 | RTA00000138A.l.5.1 |
| M00001624A:B06 | 3277 | 217.E1.sp6:139406.Seq |
| M00001624A:B06 | 3277 | 90.B4.sp6:130874.Seq |
| M00001630B:H09 | 5214 | 90.D4.sp6:130898.Seq |
| M00001630B:H09 | 5214 | 122.C2.sp6:132098.Seq |
| M00001630B:H09 | 5214 | RTA00000186AF.g.11.1 |
| M00001651A:H01 | | RTA00000186AF.n.7.1 |
| M00001651A:H01 | | 90.A5.sp6:130863.Seq |
| M00001677C:E10 | 14627 | RTA00000187AF.g.23.1 |
| M00001679C:F01 | 78091 | 90.C7.sp6:130889.Seq |
| M00001679C:F01 | 78091 | RTA00000187AF.j.6.1 |
| M00001679C:F01 | 78091 | 176.G5.sp6:134588.Seq |
| M00001686A:E06 | 4622 | RTA00000187AF.m.15.2 |
| M00003796C:D05 | 5619 | RTA00000188AF.l.9.1.Seq_THC167845 |
| M00003796C:D05 | 5619 | RTA00000188AF.l.9.1 |
| M00003826B:A06 | 11350 | RTA00000189AF.a.24.2 |
| M00003826B:A06 | 11350 | 90.F9.sp6:130927.Seq |
| M00003833A:E05 | 21877 | RTA00000189AF.b.21.1 |
| M00003837D:A01 | 7899 | 90.H9.sp6:130951.Seq |
| M00003837D:A01 | 7899 | RTA00000189AF.c.10.1 |
| M00003846B:D06 | 6874 | RTA00000189AF.e.9.1 |
| M00003846B:D06 | 6874 | 90.C10.sp6:130892.Seq |
| M00003879B:D10 | 31587 | RTA00000189AF.l.20.1 |
| M00003879B:D10 | 31587 | 90.C12.sp6:130894.Seq |
| M00003879D:A02 | 14507 | 90.D12.sp6:130906.Seq |
| M00003879D:A02 | 14507 | RTA00000189AR.l.23.2 |
| M00003891C:H09 | | 90.G12.sp6:130942.Seq |
| M00003891C:H09 | | RTA00000189AF.p.8.1 |
| M00003912B:D01 | 12532 | 99.D1.sp6:131266.Seq |
| M00003912B:D01 | 12532 | RTA00000190AF.g.2.1 |
| M00004072B:B05 | 17036 | RTA00000191AF.j.10.1 |
| M00004081C:D12 | 14391 | RTA00000191AF.l.7.1 |
| M00004111D:A08 | 6874 | RTA00000192AF.a.14.1 |
| M00004111D:A08 | 6874 | 99.F5.sp6:131294.Seq |
| M00004121B:G01 | | 177.H4.sp6:134791.Seq |
| M00004121B:G01 | | 99.H5.sp6:131318.Seq |
| M00004121B:G01 | | RTA00000192AF.c.2.1 |
| M00004138B:H02 | 13272 | 99.A6.sp6:131235.Seq |
| M00004138B:H02 | 13272 | RTA00000192AF.e.3.1 |
| M00004151D:B08 | 16977 | RTA00000192AF.g.3.1 |
| M00004169C:C12 | 5319 | 99.E6.sp6:131283.Seq |
| M00004169C:C12 | 5319 | RTA00000192AF.i.12.1 |
| M00004169C:C12 | 5319 | 123.F7.sp6:132331.Seq |
| M00004183C:D07 | 16392 | RTA00000192AF.l.1.1 |
| M00004183C:D07 | 16392 | RTA00000192AF.l.1.1.Seq_THC202071 |
| M00004230B:C07 | 7212 | RTA00000193AF.b.14.1 |
| M00004230B:C07 | 7212 | 99.D8.sp6:131273.Seq |
| M00004249D:F10 | | RTA00000193AF.c.21.1.Seq_THC222602 |
| M00004249D:F10 | | RTA00000193AF.c.21.1 |
| M00004275C:C11 | 16914 | 99.A9.sp6:131238.Seq |
| M00004275C:C11 | 16914 | RTA00000193AF.f.5.1 |
| M00004283B:A04 | 14286 | RTA00000193AF.f.22.1 |
| M00004285B:E08 | 56020 | RTA00000193AF.g.2.1 |
| M00004327B:H04 | | RTA00000193AF.j.20.1 |
| M00004377C:F05 | 2102 | RTA00000193AF.n.7.1 |
| M00004384C:D02 | | RTA00000193AF.n.15.1 |
| M00004384C:D02 | | RTA00000193AF.n.15.1.Seq_THC215687 |
| M00004461A:B08 | | RTA00000194AR.a.10.2 |
| M00004461A:B09 | | RTA00000194AF.a.11.1 |
| M00004691D:A05 | | RTA00000194AF.c.23.1 |
| M00004896A:C07 | | RTA00000194AF.d.13.1 |

The above material has been deposited with the American Type Culture Collection, Rockville, Md., under the accession number indicated. This deposit will be maintained under the terms of the Budapest Treaty on the International Recognition of the Deposit of Microorganisms for purposes of Patent Procedure. The deposit will be maintained for a period of 30 years following issuance of this patent, or for the enforceable life of the patent, whichever is greater. Upon issuance of the patent, the deposit will be available to the public from the ATCC without restriction.

This deposit is provided merely as convenience to those of skill in the art, and is not an admission that a deposit is required under 35 U.S.C. §112. The sequence of the polynucleotides contained within the deposited material, as well as the amino acid sequence of the polypeptides encoded thereby, are incorporated herein by reference and are controlling in the event of any conflict with the written description of sequences herein. A license may be required to make, use, or sell the deposited material, and no such license is granted hereby.

Retrieval of Individual Clones from Deposit of Pooled Clones

Where the ATCC deposit is composed of a pool of cDNA clones, the deposit was prepared by first transfecting each of the clones into separate bacterial cells. The clones were then deposited as a pool of equal mixtures in the composite deposit. Particular clones can be obtained from the composite deposit using methods well known in the art. For example, a bacterial cell containing a particular clone can be identified by isolating single colonies, and identifying colonies containing the specific clone through standard colony hybridization techniques, using an oligonucleotide probe or probes designed to specifically hybridize to a sequence of the clone insert (e.g., a probe based upon unmasked sequence of the encoded polynucleotide having the indicated SEQ ID NO). The probe should be designed to have a Tₘ of approximately 80° C. (assuming 2° C. for each A or T and 4° C. for each G or C). Positive colonies can then be picked, grown in culture, and the recombinant clone isolated. Alternatively, probes designed in this manner can be used to PCR to isolate a nucleic acid molecule from the pooled clones according to methods well known in the art, e.g., by purifying the cDNA from the deposited culture pool, and using the probes in PCR reactions to produce an amplified product having the corresponding desired polynucleotide sequence.

TABLE 1
Sequence identification numbers, cluster ID, sequence name, and clone name

| SEQ ID NO: | Cluster ID | Sequence Name | Clone Name |
|---|---|---|---|
| 1 | 4635 | RTA00000180AF.i.20.1 | M00001429B:A11 |
| 2 | | RTA00000185AF.n.12.1 | M00001608D:A11 |
| 3 | 4622 | RTA00000187AF.m.15.2 | M00001686A:E06 |
| 4 | 3706 | RTA00000191AF.i.17.2 | M00004068B:A01 |
| 5 | 36535 | RTA00000181AF.f.5.1 | M00001449A:G10 |
| 6 | 3990 | RTA00000183AF.j.11.1 | M00001532B:A06 |
| 7 | 5319 | RTA00000192AF.i.12.1 | M00004169C:C12 |
| 8 | 36393 | RTA00000180AF.c.2.1 | M00001417A:E02 |
| 9 | 2623 | RTA00000183AF.a.6.1 | M00001497A:G02 |
| 10 | 7587 | RTA00000178AF.n.24.1 | M00001387B:G03 |
| 11 | 7065 | RTA00000137A.g.6.1 | M00001557A:D02 |
| 12 | 10539 | RTA00000187AF.l.7.1 | M00001680D:F08 |
| 13 | 27250 | RTA00000181AF.g.10.1 | M00001450A:D08 |
| 14 | 5556 | RTA00000179AF.n.10.1 | M00001407B:D11 |
| 15 | | RTA00000192AF.m.12.1 | M00004191D:B11 |
| 16 | 8761 | RTA00000184AF.k.12.1 | M00001557D:D09 |
| 17 | 4622 | RTA00000189AF.g.1.1 | M00003856B:C02 |
| 18 | 11460 | RTA00000187AF.g.12.1 | M00001676B:F05 |
| 19 | 16283 | RTA00000120A.o.20.1 | M00001467A:D08 |
| 20 | 3430 | RTA00000191AF.a.9.1 | M00003981A:E10 |
| 21 | 7065 | RTA00000184AF.j.21.1 | M00001557A:D02 |
| 22 | | RTA00000182AF.l.20.1 | M00001488B:F12 |
| 23 | | RTA00000123A.g.19.1 | M00001531A:H11 |
| 24 | 16918 | RTA00000193AF.a.16.1 | M00004223A:G10 |
| 25 | 16914 | RTA00000193AF.f.5.1 | M00004275C:C11 |
| 26 | 40108 | RTA00000187AF.o.24.1 | M00003741D:C09 |
| 27 | 14286 | RTA00000193AF.f.22.1 | M00004283B:A04 |
| 28 | 17004 | RTA00000186AF.b.21.1 | M00001617C:E02 |
| 29 | | RTA00000180AF.g.22.1 | M00001426B:D12 |
| 30 | 13272 | RTA00000192AF.e.3.1 | M00004138B:H02 |
| 31 | | RTA00000194AF.f.4.1 | M00005180C:G03 |
| 32 | 32663 | RTA00000118A.l.8.1 | M00001450A:A11 |
| 33 | | RTA00000180AF.a.9.1 | M00001414A:B01 |
| 34 | 5832 | RTA00000178AF.o.23.1 | M00001388D:G05 |
| 35 | 7801 | RTA00000181AF.c.21.1 | M00001446A:F05 |
| 36 | 76760 | RTA00000187AF.a.15.1 | M00001657D:F08 |
| 37 | 40132 | RTA00000178AF.c.7.1 | M00001365C:C10 |
| 38 | | RTA00000183AF.e.1.1 | M00001505C:C05 |
| 39 | 4016 | RTA00000118A.c.4.1 | M00001395A:C03 |
| 40 | 5382 | RTA00000187AF.m.23.2 | M00001688C:F09 |
| 41 | 5693 | RTA00000190AF.p.17.2 | M00003978B:G05 |
| 42 | 307 | RTA00000136A.o.4.2 | M00001552A:B12 |
| 43 | 39833 | RTA00000178AF.i.23.1 | M00001378B:B02 |
| 44 | | RTA00000193AF.m.5.1 | M00004359B:G02 |
| 45 | 5325 | RTA00000191AF.o.6.1 | M00004093D:B12 |
| 46 | 5325 | RTA00000191AF.o.6.2 | M00004093D:B12 |
| 47 | 18957 | RTA00000190AR.m.9.1 | M00003958A:H02 |
| 48 | 39508 | RTA00000120A.o.2.1 | M00001467A:D04 |
| 49 | 22390 | RTA00000136A.j.13.1 | M00001551A:G06 |
| 50 | 12170 | RTA00000125A.h.18.4 | M00001544A:E03 |
| 51 | 4393 | RTA00000187AF.n.17.1 | M00001693C:G01 |
| 52 | 19 | RTA00000182AF.b.7.1 | M00001463C:B11 |
| 53 | | RTA00000193AF.c.21.1 | M00004249D:F10 |
| 54 | 7899 | RTA00000189AF.c.10.1 | M00003837D:A01 |
| 55 | 40073 | RTA00000191AF.e.3.1 | M00004028D:C05 |
| 56 | 7005 | RTA00000179AF.o.22.1 | M00001410A:D07 |
| 57 | | RTA00000187AF.h.22.1 | M00001679A:F06 |
| 58 | 18957 | RTA00000190AF.m.9.2 | M00003958A:H02 |
| 59 | 18957 | RTA00000183AF.h.23.1 | M00001528A:F09 |
| 60 | 16283 | RTA00000182AF.c.22.1 | M00001467A:D08 |
| 61 | 6974 | RTA00000183AF.d.9.1 | M00001504C:H06 |
| 62 | 2623 | RTA00000183AF.b.14.1 | M00001500A:E11 |
| 63 | 9105 | RTA00000191AF.a.21.2 | M00003983A:A05 |
| 64 | 13238 | RTA00000181AF.m.4.1 | M00001455A:E09 |
| 65 | 5749 | RTA00000185AF.a.19.1 | M00001571C:H06 |
| 66 | 6455 | RTA00000193AF.b.9.1 | M00004229B:F08 |
| 67 | 23001 | RTA00000185AF.c.24.1 | M00001578B:E04 |
| 68 | 6455 | RTA00000192AF.g.23.1 | M00004157C:A09 |
| 69 | 13595 | RTA00000189AF.f.8.1 | M00003851B:D10 |
| 70 | 39442 | RTA00000120A.o.21.1 | M00001467A:E10 |
| 71 | 17036 | RTA00000191AF.f.13.1 | M00004035D:B06 |
| 72 | | RTA00000183AF.g.9.1 | M00001513B:G03 |
| 73 | 7005 | RTA00000181AF.k.24.1 | M00001454B:C12 |
| 74 | 6268 | RTA00000126A.o.23.1 | M00001551A:B10 |
| 75 | 16130 | RTA00000119A.c.13.1 | M00001453A:E11 |
| 76 | 23201 | RTA00000187AF.a.14.1 | M00001657D:C03 |
| 77 | 5321 | RTA00000183AF.k.8.1 | M00001534A:F09 |
| 78 | 13157 | RTA00000186AF.a.6.1 | M00001614C:F10 |
| 79 | 2102 | RTA00000193AF.n.7.1 | M00004377C:F05 |
| 80 | 1058 | RTA00000126A.e.20.3 | M00001548A:H09 |
| 81 | 40392 | RTA00000180AF.j.8.1 | M00001429D:D07 |
| 82 | | RTA00000183AF.e.23.1 | M00001506D:A09 |
| 83 | 11476 | RTA00000187AF.p.19.1 | M00003747D:C05 |
| 84 | 3584 | RTA00000177AF.h.20.1 | M00001349B:B08 |
| 85 | 10470 | RTA00000180AF.f.18.1 | M00001424B:G09 |
| 86 | 39425 | RTA00000133A.f.1.1 | M00001470A:C04 |
| 87 | 5175 | RTA00000184AF.f.3.1 | M00001550A:G01 |
| 88 | 13576 | RTA00000189AF.o.13.1 | M00003885C:A02 |
| 89 | 7665 | RTA00000134A.l.19.1 | M00001535A:B01 |
| 90 | 16927 | RTA00000177AF.h.9.3 | M00001348B:B04 |
| 91 | 6660 | RTA00000187AF.h.15.1 | M00001679A:A06 |
| 92 | 2433 | RTA00000191AF.a.15.2 | M00003982C:C02 |
| 93 | 5097 | RTA00000134A.k.1.1 | M00001534A:D09 |
| 94 | 21847 | RTA00000193AF.j.9.1 | M00004318C:D10 |
| 95 | 3277 | RTA00000138A.l.5.1 | M00001624A:806 |
| 96 | 5708 | RTA00000184AF.g.12.1 | M00001552B:D04 |
| 97 | 945 | RTA00000178AR.a.20.1 | M00001362C:H11 |
| 98 | 16269 | RTA00000178AF.p.1.1 | M00001389A:C08 |
| 99 | | RTA00000183AF.c.24.1 | M00001504A:E01 |
| 100 | 16731 | RTA00000181AF.a.20.1 | M00001442C:D07 |
| 101 | 12439 | RTA00000190AF.o.24.1 | M00003975A:G11 |
| 102 | 3162 | RTA00000177AF.j.12.3 | M00001351B:A08 |
| 103 | | RTA00000194AF.b.19.1 | M00004505D:F08 |
| 104 | | RTA00000193AF.n.15.1 | M00004384C:D02 |
| 105 | | RTA00000186AF.n.7.1 | M00001651A:H01 |
| 106 | 10717 | RTA00000181AF.d.10.1 | M00001447A:G03 |
| 107 | 4573 | RTA00000189AF.j.12.1 | M00003871C:E02 |
| 108 | | RTA00000186AF.h.14.1 | M00001632D:H07 |
| 109 | 11443 | RTA00000192AF.l.13.2 | M00004185C:C03 |
| 110 | 5892 | RTA00000184AF.d.11.1 | M00001548A:E10 |
| 111 | 3162 | RTA00000177AF.j.12.1 | M00001351B:A08 |
| 112 | 10470 | RTA00000185AF.k.6.1 | M00001597D:C05 |
| 113 | 17055 | RTA00000187AF.m.3.1 | M00001682C:B12 |
| 114 | 2030 | RTA00000193AF.m.20.1 | M00004372A:A03 |
| 115 | 6558 | RTA00000184AF.m.21.1 | M00001560D:F10 |
| 116 | 23255 | RTA00000190AF.j.4.1 | M00003922A:E06 |
| 117 | 9577 | RTA00000179AF.o.17.1 | M00001409C:D12 |
| 118 | | RTA00000180AF.a.11.1 | M00001414C:A07 |
| 119 | 8 | RTA00000181AF.e.17.1 | M00001448D:C09 |
| 120 | 67907 | RTA00000188AF.g.11.1 | M00003774C:A03 |
| 121 | 12081 | RTA00000133A.d.14.2 | M00001469A:C10 |
| 122 | 2448 | RTA00000119A.j.21.1 | M00001460A:F06 |
| 123 | 3389 | RTA00000189AF.g.3.1 | M00003857A:G10 |
| 124 | 39174 | RTA00000124A.n.13.1 | M00001541A:H03 |
| 125 | 24488 | RTA00000190AF.n.16.1 | M00003968B:F06 |
| 126 | 8210 | RTA00000192AF.n.13.1 | M00004197D:H01 |
| 127 | | RTA00000135A.l.2.2 | M00001545A:B02 |
| 128 | 40455 | RTA00000190AF.m.10.2 | M00003958C:G10 |
| 129 | 9577 | RTA00000180AF.d.23.1 | M00001421C:F01 |
| 130 | 13183 | RTA00000192AF.a.24.1 | M00004114C:F11 |
| 131 | 5214 | RTA00000186AF.g.11.1 | M00001630B:H09 |
| 132 | 67252 | RTA00000187AF.o.6.1 | M00001716D:H05 |
| 133 | 3108 | RTA00000188AF.d.24.1 | M00003763A:F06 |
| 134 | 2464 | RTA00000178AF.n.18.1 | M00001387A:C05 |
| 135 | 36313 | RTA00000181AF.e.23.1 | M00001448D:H01 |
| 136 | 23255 | RTA00000177AF.e.14.3 | M00001343D:H07 |
| 137 | 7985 | RTA00000182AR.j.2.1 | M00001481D:A05 |
| 138 | 8286 | RTA00000183AF.o.1.1 | M00001540A:D06 |
| 139 | 22195 | RTA00000180AF.g.7.1 | M00001425B:H08 |
| 140 | 4573 | RTA00000184AF.h.9.1 | M00001553B:F12 |
| 141 | 26875 | RTA00000187AF.i.1.1 | M00001679A:F10 |
| 142 | 7187 | RTA00000177AF.i.8.2 | M00001350A:H01 |
| 143 | 86859 | RTA00000118A.p.8.1 | M00001452A:B12 |
| 144 | 4623 | RTA00000185AF.f.4.1 | M00001586C:C05 |
| 145 | | RTA00000121A.c.10.1 | M00001469A:A01 |
| 146 | 10185 | RTA00000183AF.d.5.1 | M00001504C:A07 |
| 147 | | RTA00000183AF.p.4.1 | M00001542B:B01 |
| 148 | 15069 | RTA00000191AF.l.6.1 | M00004081C:D10 |
| 149 | 39304 | RTA00000118A.j.21.1 | M00001450A:A02 |
| 150 | 8672 | RTA00000190AF.f.11.1 | M00003909D:C03 |
| 151 | 13576 | RTA00000177AF.g.16.1 | M00001347A:B10 |
| 152 | 6293 | RTA00000185AF.e.11.1 | M00001583D:A10 |
| 153 | 16977 | RTA00000192AF.g.3.1 | M00004151D:B08 |
| 154 | 5345 | RTA00000189AF.l.19.1 | M00003879B:C11 |
| 155 | 4905 | RTA00000193AF.e.14.1 | M00004269D:D06 |
| 156 | 17036 | RTA00000191AF.j.10.1 | M00004072B:B05 |
| 157 | 5417 | RTA00000191AF.h.19.1 | M00004059A:D06 |
| 158 | 7172 | RTA00000178AF.f.9.1 | M00001371C:E09 |
| 159 | 40044 | RTA00000186AF.d.1.1 | M00001621C:C08 |
| 160 | 4386 | RTA00000184AF.j.4.1 | M00001556B:C08 |
| 161 | 40044 | RTA00000183AF.g.22.1 | M00001514C:D11 |
| 162 | 9685 | RTA00000183AF.c.11.1 | M00001501D:C02 |
| 163 | 22155 | RTA00000185AF.n.9.1 | M00001608B:E03 |
| 164 | 10515 | RTA00000189AF.f.18.1 | M00003853A:F12 |
| 165 | 6539 | RTA00000185AF.d.11.1 | M00001579D:C03 |
| 166 | 15066 | RTA00000180AF.e.24.1 | M00001423B:E07 |
| 167 | 4261 | RTA00000180AF.h.5.1 | M00001426D:C08 |
| 168 | 13864 | RTA00000125A.m.9.1 | M00001545A:D08 |
| 169 | 6539 | RTA00000189AF.d.22.1 | M00003844C:B11 |
| 170 | 11465 | RTA00000185AF.m.19.1 | M00001607A:E11 |
| 171 | 3266 | RTA00000184AR.g.1.1 | M00001551C:G09 |
| 172 | 102 | RTA00000184AF.o.5.1 | M00001563B:F06 |
| 173 | 16970 | RTA00000181AR.i.18.2 | M00001452C:B06 |
| 174 | 12971 | RTA00000193AF.a.20.1 | M00004223D:E04 |
| 175 | 5007 | RTA00000177AF.g.2.1 | M00001346A:F09 |
| 176 | 3765 | RTA00000135A.d.1.1 | M00001541A:D02 |
| 177 | 11294 | RTA00000184AF.j.6.1 | M00001556B:G02 |
| 178 | 3681 | RTA00000131A.g.15.2 | M00001449A:D12 |
| 179 | 9283 | RTA00000181AR.m.21.2 | M00001455D:F09 |
| 180 | 18699 | RTA00000182AF.m.16.1 | M00001490B:C04 |
| 181 | 86110 | RTA00000181AF.f.12.1 | M00001449C:D06 |
| 182 | 39648 | RTA00000178AR.l.8.2 | M00001383A:C03 |
| 183 | 7337 | RTA00000123A.b.17.1 | M00001528A:C04 |
| 184 | 1334 | RTA00000178AF.j.7.1 | M00001379A:A05 |
| 185 | 17076 | RTA00000188AF.d.21.1 | M00003762C:B08 |
| 186 | 22794 | RTA00000138A.b.5.1 | M00001601A:D08 |
| 187 | 39171 | RTA00000186AF.l.7.1 | M00001644C:B07 |
| 188 | 8551 | RTA00000179AF.p.21.1 | M00001412B:B10 |
| 189 | 5857 | RTA00000118A.g.14.1 | M00001449A:A12 |
| 190 | 9443 | RTA00000183AF.c.1.1 | M00001500C:E04 |
| 191 | 9457 | RTA00000193AF.i.14.2 | M00004307C:A06 |
| 192 | 7206 | RTA00000182AF.o.15.1 | M00001494D:F06 |
| 193 | 22979 | RTA00000178AF.k.22.1 | M00001382C:A02 |
| 194 | 40455 | RTA00000190AR.m.10.1 | M00003958C:G10 |
| 195 | 7221 | RTA00000191AF.p.9.1 | M00004105C:A04 |
| 196 | | RTA00000191AF.j.9.1 | M00004072A:C03 |
| 197 | 7239 | RTA00000126A.m.4.2 | M00001550A:A03 |
| 198 | 31587 | RTA00000189AF.l.20.1 | M00003879B:D10 |
| 199 | 16317 | RTA00000190AF.e.6.1 | M00003907D:H04 |
| 200 | 13576 | RTA00000189AR.o.13.1 | M00003885C:A02 |
| 201 | 5779 | RTA00000177AF.g.14.3 | M00001346D:G06 |
| 202 | 6124 | RTA00000191AR.e.2.3 | M00004028D:A06 |
| 203 | 9952 | RTA00000180AF.c.20.1 | M00001418B:F03 |
| 204 | | RTA00000188AF.i.8.1 | M00003784D:D12 |
| 205 | 5779 | RTA00000177AF.g.14.1 | M00001346D:G06 |
| 206 | 39490 | RTA00000128A.b.4.1 | M00001557A:F03 |
| 207 | 4416 | RTA00000187AF.h.13.1 | M00001678D:F12 |
| 208 | 4009 | RTA00000179AF.e.20.1 | M00001396A:C03 |
| 209 | 5336 | RTA00000183AF.b.13.1 | M00001500A:C05 |
| 210 | 39186 | RTA00000121A.p.15.1 | M00001512A:A09 |
| 211 | 40122 | RTA00000190AF.n.23.1 | M00003970C:B09 |
| 212 | 12532 | RTA00000190AF.g.2.1 | M00003912B:D01 |
| 213 | 8078 | RTA00000177AR.l.13.1 | M00001353A:G12 |
| 214 | 3900 | RTA00000190AF.g.13.1 | M00003914C:F05 |
| 215 | 7589 | RTA00000120A.p.23.1 | M00001468A:F05 |
| 216 | 8298 | RTA00000127A.d.19.1 | M00001553A:H06 |
| 217 | 4443 | RTA00000177AF.b.20.4 | M00001341A:E12 |
| 218 | 26295 | RTA00000193AF.i.24.2 | M00004312A:G03 |
| 219 | 3389 | RTA00000183AF.m.19.1 | M00001537B:G07 |
| 220 | 7015 | RTA00000187AF.f.18.1 | M00001673C:H02 |
| 221 | 8526 | RTA00000180AF.d.1.1 | M00001418D:B06 |
| 222 | 4665 | RTA00000186AF.m.3.1 | M00001648C:A01 |
| 223 | 1399 | RTA00000129A.o.10.1 | M00001604A:B10 |
| 224 | 9244 | RTA00000127A.l.3.1 | M00001556A:C09 |
| 225 | | RTA00000179AF.j.13.1 | M00001400B:H06 |
| 226 | 82498 | RTA00000118A.m.10.1 | M00001450A:B12 |
| 227 | 35702 | RTA00000187AR.c.15.2 | M00001663A:E04 |
| 228 | 38759 | RTA00000120A.m.12.3 | M00001467A:B07 |
| 229 | 39648 | RTA00000178AF.l.8.1 | M00001383A:C03 |
| 230 | 19105 | RTA00000133A.e.15.1 | M00001469A:H12 |
| 231 | 85064 | RTA00000131A.m.23.1 | M00001452A:F05 |
| 232 | 9285 | RTA00000191AF.m.18.1 | M00004086D:G06 |
| 233 | 9285 | RTA00000190AF.d.7.1 | M00003906C:E10 |
| 234 | 39391 | RTA00000138A.c.3.1 | M00001604A:F05 |
| 235 | | RTA00000178AF.d.20.1 | M00001368D:E03 |
| 236 | 39498 | RTA00000119A.j.20.1 | M00001460A:F12 |
| 237 | 7798 | RTA00000189AF.k.12.1 | M00003876D:E12 |
| 238 | 7798 | RTA00000189AF.c.18.1 | M00003839A:D08 |
| 239 | 19829 | RTA00000125A.h.24.4 | M00001544A:G02 |
| 240 | | RTA00000188AF.d.11.1 | M00003761D:A09 |
| 241 | 4275 | RTA00000120A.j.14.1 | M00001466A:E07 |
| 242 | 22113 | RTA00000125A.c.7.1 | M00001542A:A09 |
| 243 | 40314 | RTA00000186AF.c.15.1 | M00001619C:F12 |
| 244 | 10944 | RTA00000126A.h.17.2 | M00001549A:D08 |
| 245 | 39809 | RTA00000190AF.e.3.1 | M00003907D:A09 |
| 246 | 22085 | RTA00000135A.e.5.2 | M00001541A:F07 |
| 247 | 19255 | RTA00000135A.m.18.1 | M00001545A:C03 |
| 248 | 14311 | RTA00000192AF.o.2.1 | M00004203B:C12 |
| 249 | 8479 | RTA00000189AF.j.22.1 | M00003875C:G07 |
| 250 | | RTA00000189AF.j.23.1 | M00003875D:D11 |
| 251 | 4193 | RTA00000184AF.e.13.1 | M00001549B:F06 |
| 252 | 22814 | RTA00000184AF.h.14.1 | M00001553D:D10 |
| 253 | 39563 | RTA00000179AF.k.20.1 | M00001402A:E08 |
| 254 | 39420 | RTA00000134A.o.23.1 | M00001537A:F12 |
| 255 | 11589 | RTA00000177AF.b.17.4 | M00001340D:F10 |
| 256 | 4937 | RTA00000191AF.p.21.1 | M00004108A:E06 |
| 257 | 39412 | RTA00000133A.k.17.1 | M00001511A:H06 |
| 258 | 4837 | RTA00000185AR.k.3.2 | M00001597C:H02 |
| 259 | 13046 | RTA00000193AF.h.19.1 | M00004296C:H07 |
| 260 | 4141 | RTA00000177AF.p.20.3 | M00001361A:A05 |
| 261 | 38085 | RTA00000123A.e.15.1 | M00001531A:D01 |
| 262 | | RTA00000189AF.p.8.1 | M00003891C:H09 |
| 263 | 11451 | RTA00000192AF.p.17.1 | M00004214C:H05 |
| 264 | 14507 | RTA00000189AR.l.23.2 | M00003879D:A02 |
| 265 | 40054 | RTA00000180AF.p.10.1 | M00001439C:F08 |
| 266 | 39423 | RTA00000134A.k.22.1 | M00001535A:F10 |
| 267 | 39453 | RTA00000135A.g.11.1 | M00001542A:E06 |
| 268 | 10751 | RTA00000187AF.k.7.1 | M00001679D:D03 |
| 269 | 10751 | RTA00000187AF.k.6.1 | M00001679D:D03 |
| 270 | 78091 | RTA00000187AF.j.6.1 | M00001679C:F01 |
| 271 | 39539 | RTA00000127A.i.21.1 | M00001555A:B02 |
| 272 | | RTA00000182AF.l.15.1 | M00001487B:H06 |
| 273 | | RTA00000194AF.d.13.1 | M00004896A:C07 |
| 274 | | RTA00000128A.c.20.1 | M00001558A:H05 |
| 275 | 9283 | RTA00000181AR.m.22.2 | M00001455D:F09 |
| 276 | 39168 | RTA00000121A.l.10.1 | M00001507A:H05 |
| 277 | 39458 | RTA00000126A.p.15.2 | M00001552A:D11 |
| 278 | 14391 | RTA00000177AF.m.17.3 | M00001355B:G10 |
| 279 | 39195 | RTA00000137A.c.16.1 | M00001555A:C01 |
| 280 | 7212 | RTA00000193AF.b.14.1 | M00004230B:C07 |
| 281 | 4015 | RTA00000136A.e.12.1 | M00001549A:B02 |
| 282 | 12977 | RTA00000189AF.j.19.1 | M00003875B:F04 |
| 283 | | RTA00000178AF.m.13.1 | M00001384B:A11 |
| 284 | 14391 | RTA00000191AF.l.7.1 | M00004081C:D12 |
| 285 | | RTA00000194AF.c.23.1 | M00004691D:A05 |
| 286 | | RTA00000181AF.b.7.1 | M00001443B:F01 |
| 287 | 8358 | RTA00000183AF.i.5.1 | M00001528B:H04 |
| 288 | 1267 | RTA00000125A.o.5.1 | M00001546A:G11 |
| 289 | | RTA00000189AF.f.7.1 | M00003851B:D08 |
| 290 | 16347 | RTA00000184AF.e.15.1 | M00001549C:E06 |
| 291 | 7899 | RTA00000193AF.a.17.1 | M00004223B:D09 |
| 292 | 2379 | RTA00000178AF.a.6.1 | M00001361D:F08 |
| 293 | 39478 | RTA00000133A.i.5.1 | M00001471A:B01 |
| 294 | 39392 | RTA00000134A.m.16.1 | M00001536A:C08 |
| 295 | 5053 | RTA00000184AF.o.12.1 | M00001564A:B12 |
| 296 | 16999 | RTA00000185AF.k.9.1 | M00001598A:G03 |
| 297 | 39180 | RTA00000126A.n.8.2 | M00001551A:F05 |
| 298 | 1037 | RTA00000121A.f.8.1 | M00001470A:B10 |
| 299 | 6867 | RTA00000178AF.e.12.1 | M00001370A:C09 |
| 300 | 10539 | RTA00000183AF.a.24.1 | M00001499B:A11 |
| 301 | 41633 | RTA00000118A.g.16.1 | M00001449A:B12 |
| 302 | 23218 | RTA00000187AR.c.5.2 | M00001662C:A09 |
| 303 | 39380 | RTA00000129A.e.24.1 | M00001587A:B11 |
| 304 | | RTA00000185AF.d.24.1 | M00001582D:F05 |
| 305 | | RTA00000177AF.o.4.3 | M00001358C:C06 |
| 306 | 6974 | RTA00000184AF.a.15.1 | M00001544B:B07 |
| 307 | | RTA00000185AF.g.11.1 | M00001590B:F03 |
| 308 | 15855 | RTA00000184AF.j.1.1 | M00001556A:H01 |
| 309 | 84328 | RTA00000118A.p.10.1 | M00001452A:B04 |
| 310 | 10145 | RTA00000120A.g.12.1 | M00001465A:B11 |
| 311 | 39805 | RTA00000177AF.c.21.3 | M00001342B:E06 |
| 312 | | RTA00000187AF.h.23.1 | M00001679A:F06 |
| 313 | 6298 | RTA00000187AR.i.10.2 | M00001679B:F01 |
| 314 | 14367 | RTA00000187AF.e.8.1 | M00001670C:H02 |
| 315 | | RTA00000193AF.c.22.1 | M00004249D:G12 |
| 316 | 16921 | RTA00000183AF.k.6.1 | M00001534A:C04 |
| 317 | 1577 | RTA00000184AF.i.23.1 | M00001556A:F11 |
| 318 | 8773 | RTA00000187AF.f.24.1 | M00001675A:C09 |
| 319 | | RTA00000194AF.a.11.1 | M00004461A:B09 |
| 320 | 39886 | RTA00000178AF.j.24.1 | M00001380D:B09 |
| 321 | 13532 | RTA00000181AF.c.4.1 | M00001445A:F05 |
| 322 | | RTA00000193AF.d.2.1 | M00004251C:G07 |
| 323 | 5257 | RTA00000192AF.f.3.1 | M00004146C:C11 |
| 324 | 9061 | RTA00000191AR.e.11.2 | M00004031A:A12 |
| 325 | 19267 | RTA00000186AF.l.12.1 | M00001645A:C12 |
| 326 | 20212 | RTA00000134A.l.22.1 | M00001535A:C06 |
| 327 | 16653 | RTA00000181AF.k.5.3 | M00001453C:F06 |
| 328 | 16985 | RTA00000177AF.h.10.1 | M00001348B:G06 |
| 329 | 12977 | RTA00000189AR.j.19.1 | M00003875B:F04 |
| 330 | 9061 | RTA00000191AR.e.11.3 | M00004031A:A12 |
| 331 | | RTA00000194AR.a.10.2 | M00004461A:B08 |
| 332 | 6468 | RTA00000187AF.d.15.1 | M00001669B:F02 |
| 333 | 16392 | RTA00000192AF.l.1.1 | M00004183C:D07 |
| 334 | 14627 | RTA00000187AF.g.23.1 | M00001677C:E10 |
| 335 | 6583 | RTA00000179AF.d.13.1 | M00001394A:F01 |
| 336 | 6806 | RTA00000177AF.g.13.3 | M00001346D:E03 |
| 337 | 9635 | RTA00000137A.e.23.4 | M00001557A:F01 |
| 338 | 689 | RTA00000181AR.l.22.1 | M00001454D:G03 |
| 339 | 4119 | RTA00000183AF.k.16.1 | M00001534C:A01 |
| 340 | 8952 | RTA00000183AF.h.15.1 | M00001518C:B11 |
| 341 | 2379 | RTA00000192AF.p.8.1 | M00004212B:C07 |
| 342 | 39486 | RTA00000128A.m.22.2 | M00001561A:C05 |
| 343 | 21877 | RTA00000189AF.b.21.1 | M00003833A:E05 |
| 344 | 6874 | RTA00000192AF.a.14.1 | M00004111D:A08 |
| 345 | 6874 | RTA00000189AF.e.9.1 | M00003846B:D06 |
| 346 | 37285 | RTA00000191AF.f.11.1 | M00004035C:A07 |
| 347 | | RTA00000193AF.j.20.1 | M00004327B:H04 |
| 348 | 7674 | RTA00000118A.g.9.1 | M00001416A:H01 |
| 349 | 2797 | RTA00000180AF.i.19.1 | M00001429A:H04 |
| 350 | | RTA00000184AF.g.22.1 | M00001552D:A01 |
| 351 | 7802 | RTA00000185AF.n.5.1 | M00001608A:B03 |
| 352 | 16921 | RTA00000193AF.h.15.1 | M00004295D:F12 |
| 353 | 11494 | RTA00000192AF.j.6.1 | M00004172C:D08 |
| 354 | 17062 | RTA00000177AF.b.8.4 | M00001340B:A06 |
| 355 | 16245 | RTA00000177AF.k.9.3 | M00001352A:E02 |
| 356 | 83103 | RTA00000119A.e.24.2 | M00001454A:A09 |
| 357 | 4309 | RTA00000186AF.e.22.1 | M00001624C:F01 |
| 358 | 13072 | RTA00000181AR.m.5.2 | M00001455B:E12 |
| 359 | 4059 | RTA00000177AF.n.18.3 | M00001357D:D11 |
| 360 | 5178 | RTA00000178AF.n.10.1 | M00001386C:B12 |
| 361 | 1120 | RTA00000118A.p.15.3 | M00001452A:D08 |
| 362 | 6420 | RTA00000183AF.d.11.1 | M00001504D:G06 |
| 363 | 13913 | RTA00000186AF.e.6.1 | M00001623D:F10 |
| 364 | | RTA00000192AF.c.2.1 | M00004121B:G01 |
| 365 | 3956 | RTA00000183AF.g.3.1 | M00001512D:G09 |
| 366 | 14364 | RTA00000183AF.g.12.1 | M00001513C:E08 |
| 367 | 6880 | RTA00000191AF.m.20.1 | M00004087D:A01 |
| 368 | 84182 | RTA00000180AF.h.19.1 | M00001428A:H10 |
| 369 | 2790 | RTA00000177AF.e.2.1 | M00001343C:F10 |
| 370 | 4561 | RTA00000184AF.i.21.1 | M00001555D:G10 |
| 371 | 8847 | RTA00000180AF.b.16.1 | M00001416B:H11 |
| 372 | 56020 | RTA00000193AF.g.2.1 | M00004285B:E08 |
| 373 | 1531 | RTA00000119A.o.3.1 | M00001461A:D06 |
| 374 | 6420 | RTA00000177AF.f.10.3 | M00001345A:E01 |
| 375 | | RTA00000188AF.b.12.1 | M00003754C:E09 |
| 376 | | RTA00000180AF.k.24.1 | M00001432C:F06 |
| 377 | | RTA00000184AF.a.8.1 | M00001544A:E06 |
| 378 | 2696 | RTA00000134A.m.13.1 | M00001536A:B07 |
| 379 | 260 | RTA00000185AR.i.12.2 | M00001594B:H04 |
| 380 | 11350 | RTA00000189AF.a.24.2 | M00003826B:A06 |
| 381 | 2428 | RTA00000123A.l.21.1 | M00001533A:C11 |
| 382 | 4313 | RTA00000122A.n.3.1 | M00001517A:B07 |
| 383 | | RTA00000184AF.p.3.1 | M00001566B:D11 |
| 384 | 697 | RTA00000188AF.d.6.1 | M00003759B:B09 |
| 385 | 5619 | RTA00000188AF.l.9.1 | M00003796C:D05 |
| 386 | 4568 | RTA00000122A.d.15.3 | M00001513A:B06 |
| 387 | | RTA00000177AF.i.6.2 | M00001350A:B08 |
| 388 | 5622 | RTA00000178AF.a.11.1 | M00001362B:D10 |
| 389 | 7514 | RTA00000184AF.k.21.1 | M00001558B:H11 |
| 390 | 5619 | RTA00000189AF.f.17.1 | M00003853A:D04 |
| 391 | 7570 | RTA00000187AF.g.24.1 | M00001677D:A07 |
| 392 | 23358 | RTA00000190AF.o.21.1 | M00003974D:H02 |
| 393 | 23210 | RTA00000190AF.o.20.1 | M00003974D:E07 |
| 394 | 5192 | RTA00000184AF.k.2.1 | M00001557B:H10 |
| 395 | 13538 | RTA00000180AF.a.24.1 | M00001415A:H06 |
| 396 | | RTA00000189AF.h.17.1 | M00003867A:D10 |
| 397 | | RTA00000192AF.o.11.1 | M00004205D:F06 |
| 398 | | RTA00000184AF.l.11.1 | M00001559B:F01 |
| 399 | 4718 | RTA00000189AF.g.5.1 | M00003857A:H03 |
| 400 | 14929 | RTA00000177AF.m.1.2 | M00001353D:D10 |
| 401 | 4908 | RTA00000192AF.j.2.1 | M00004171D:B03 |
| 402 | | RTA00000178AF.k.16.1 | M00001381D:E06 |
| 403 | | RTA00000194AF.c.24.1 | M00004692A:H08 |
| 404 | 17732 | RTA00000178AR.i.2.2 | M00001376B:G06 |
| 405 | 17062 | 80.A1.sp6:130208.Seq | M00001340B:A06 |
| 406 | 11589 | 80.B1.sp6:130220.Seq | M00001340D:F10 |
| 407 | 4443 | 80.C1.sp6:130232.Seq | M00001341A:E12 |
| 408 | 39805 | 80.D1.sp6:130244.Seq | M00001342B:E06 |
| 409 | 2790 | 80.E1.sp6:130256.Seq | M00001343C:F10 |
| 410 | 23255 | 80.F1.sp6:130268.Seq | M00001343D:H07 |
| 411 | 6420 | 80.G1.sp6:130280.Seq | M00001345A:E01 |
| 412 | 5007 | 80.H1.sp6:130292.Seq | M00001346A:F09 |
| 413 | 13576 | 80.D2.sp6:130245.Seq | M00001347A:B10 |
| 414 | 16927 | 80.E2.sp6:130257.Seq | M00001348B:B04 |
| 415 | 16985 | 80.F2.sp6:130269.Seq | M00001348B:G06 |
| 416 | 3584 | 80.G2.sp6:130281.Seq | M00001349B:B08 |
| 417 | | 80.H2.sp6:130293.Seq | M00001350A:B08 |
| 418 | 7187 | 80.A3.sp6:130210.Seq | M00001350A:H01 |
| 419 | 16245 | 80.D3.sp6:130246.Seq | M00001352A:E02 |
| 420 | 8078 | 80.E3.sp6:130258.Seq | M00001353A:G12 |
| 421 | 14929 | 80.F3.sp6:130270.Seq | M00001353D:D10 |
| 422 | 14391 | 80.G3.sp6:130282.Seq | M00001355B:G10 |
| 423 | 4141 | 80.B4.sp6:130223.Seq | M00001361A:A05 |
| 424 | 2379 | 80.C4.sp6:130235.Seq | M00001361D:F08 |
| 425 | 5622 | 80.D4.sp6:130247.Seq | M00001362B:D10 |
| 426 | 945 | 80.E4.sp6:130259.Seq | M00001362C:H11 |
| 427 | 40132 | 80.F4.sp6:130271.Seq | M00001365C:C10 |
| 428 | | 80.G4.sp6:130283.Seq | M00001368D:E03 |
| 429 | 6867 | 80.H4.sp6:130295.Seq | M00001370A:C09 |
| 430 | 7172 | 80.A5.sp6:130212.Seq | M00001371C:E09 |
| 431 | 17732 | 80.B5.sp6:130224.Seq | M00001376B:G06 |
| 432 | 39833 | 80.C5.sp6:130236.Seq | M00001378B:B02 |
| 433 | 1334 | 80.D5.sp6:130248.Seq | M00001379A:A05 |
| 434 | 39886 | 80.E5.sp6:130260.Seq | M00001380D:B09 |
| 435 | | 80.F5.sp6:130272.Seq | M00001381D:E06 |
| 436 | 22979 | 80.G5.sp6:130284.Seq | M00001382C:A02 |
| 437 | 39648 | 80.H5.sp6:130296.Seq | M00001383A:C03 |
| 438 | | 80.B6.sp6:130225.Seq | M00001384B:A11 |
| 439 | 5178 | 80.C6.sp6:130237.Seq | M00001386C:B12 |
| 440 | 2464 | 80.D6.sp6:130249.Seq | M00001387A:C05 |
| 441 | 7587 | 80.E6.sp6:130261.Seq | M00001387B:G03 |
| 442 | 5832 | 80.F6.sp6:130273.Seq | M00001388D:G05 |
| 443 | 16269 | 80.G6.sp6:130285.Seq | M00001389A:C08 |
| 444 | 6583 | 80.H6.sp6:130297.Seq | M00001394A:F01 |
| 445 | 4009 | 80.A7.sp6:130214.Seq | M00001396A:C03 |
| 446 | | 80.B7.sp6:130226.Seq | M00001400B:H06 |
| 447 | 39563 | 80.C7.sp6:130238.Seq | M00001402A:E08 |
| 448 | 5556 | 80.D7.sp6:130250.Seq | M00001407B:D11 |
| 449 | 9577 | 80.E7.sp6:130262.Seq | M00001409C:D12 |
| 450 | 7005 | 80.F7.sp6:130274.Seq | M00001410A:D07 |
| 451 | 8551 | 80.G7.sp6:130286.Seq | M00001412B:B10 |
| 452 | | 80.H7.sp6:130298.Seq | M00001414A:B01 |
| 453 | | 80.A8.sp6:130215.Seq | M00001414C:A07 |
| 454 | 13538 | 80.B8.sp6:130227.Seq | M00001415A:H06 |
| 455 | 8847 | 80.C8.sp6:130239.Seq | M00001416B:H11 |
| 456 | 36393 | 80.D8.sp6:130251.Seq | M00001417A:E02 |
| 457 | 9952 | 80.E8.sp6:130263.Seq | M00001418B:F03 |
| 458 | 9577 | 80.G8.sp6:130287.Seq | M00001421C:F01 |
| 459 | 15066 | 80.H8.sp6:130299.Seq | M00001423B:E07 |
| 460 | 10470 | 80.A9.sp6:130216.Seq | M00001424B:G09 |
| 461 | 22195 | 80.B9.sp6:130228.Seq | M00001425B:H08 |
| 462 | | 80.C9.sp6:130240.Seq | M00001426B:D12 |
| 463 | 4261 | 80.D9.sp6:130252.Seq | M00001426D:C08 |
| 464 | 84182 | 80.E9.sp6:130264.Seq | M00001428A:H10 |
| 465 | 40392 | 80.H9.sp6:130300.Seq | M00001429D:D07 |
| 466 | 16731 | 80.C10.sp6:130241.Seq | M00001442C:D07 |
| 467 | | 80.D10.sp6:130253.Seq | M00001443B:F01 |
| 468 | 13532 | 80.E10.sp6:130265.Seq | M00001445A:F05 |
| 469 | 8 | 80.H10.sp6:130301.Seq | M00001448D:C09 |
| 470 | 36313 | 80.A11.sp6:130218.Seq | M00001448D:H01 |
| 471 | 5857 | 80.B11.sp6:130230.Seq | M00001449A:A12 |
| 472 | 41633 | 80.C11.sp6:130242.Seq | M00001449A:B12 |
| 473 | 36535 | 80.D11.sp6:130254.Seq | M00001449A:G10 |
| 474 | 86110 | 80.E11.sp6:130266.Seq | M00001449C:D06 |
| 475 | 32663 | 80.F11.sp6:130278.Seq | M00001450A:A11 |
| 476 | 27250 | 80.G11.sp6:130290.Seq | M00001450A:D08 |
| 477 | 16970 | 80.H11.sp6:130302.Seq | M00001452C:B06 |
| 478 | 16130 | 80.A12.sp6:130219.Seq | M00001453A:E11 |
| 479 | 16653 | 80.B12.sp6:130231.Seq | M00001453C:F06 |
| 480 | 7005 | 80.C12.sp6:130243.Seq | M00001454B:C12 |
| 481 | 13072 | 80.F12.sp6:130279.Seq | M00001455B:E12 |
| 482 | 9283 | 80.G12.sp6:130291.Seq | M00001455D:F09 |
| 483 | 23255 | 100.C1.sp6:131446.Seq | M00001343D:H07 |
| 484 | 13576 | 100.E1.sp6:131470.Seq | M00001347A:B10 |
| 485 | 7187 | 100.C2.sp6:131447.Seq | M00001350A:H01 |
| 486 | 14391 | 100.E3.sp6:131472.Seq | M00001355B:G10 |
| 487 | 945 | 100.E4.sp6:131473.Seq | M00001362C:H11 |
| 488 | 7172 | 100.A5.sp6:131426.Seq | M00001371C:E09 |
| 489 | 39648 | 100.A6.sp6:131427.Seq | M00001383A:C03 |
| 490 | 84182 | 100.G9.sp6:131502.Seq | M00001428A:H10 |
| 491 | 8 | 100.B11.sp6:131444.Seq | M00001448D:C09 |
| 492 | 36535 | 100.D11.sp6:131468.Seq | M00001449A:G10 |
| 493 | 82498 | 100.F11.sp6:131492.Seq | M00001450A:B12 |
| 494 | 16970 | 100.C12.sp6:131457.Seq | M00001452C:B06 |
| 495 | 16130 | 100.D12.sp6:131469.Seq | M00001453A:E11 |
| 496 | 7005 | 121.D1.sp6:131917.Seq | M00001454B:C12 |
| 497 | | 121.G6.sp6:131958.Seq | M00001506D:A09 |
| 498 | 18957 | 121.F7.sp6:131947.Seq | M00001528A:F09 |
| 499 | 40044 | 122.E1.sp6:132121.Seq | M00001621C:C08 |
| 500 | 5214 | 122.C2.sp6:132098.Seq | M00001630B:H09 |
| 501 | 6660 | 122.B5.sp6:132089.Seq | M00001679A:A06 |
| 502 | 13183 | 123.D5.sp6:132305.Seq | M00004114C:F11 |
| 503 | 6455 | 123.E7.sp6:132319.Seq | M00004157C:A09 |
| 504 | 5319 | 123.F7.sp6:132331.Seq | M00004169C:C12 |
| 505 | 11443 | 123.A8.sp6:132272.Seq | M00004185C:C03 |
| 506 | | 123.C8.sp6:132296.Seq | M00004191D:B11 |
| 507 | 8210 | 123.E8.sp6:132320.Seq | M00004197D:H01 |
| 508 | 9457 | 123.D11.sp6:132311.Seq | M00004307C:A06 |
| 509 | 6420 | 172.E1.sp6:133925.Seq | M00001345A:E01 |
| 510 | 16245 | 172.D2.sp6:133914.Seq | M00001352A:E02 |
| 511 | 8078 | 172.C3.sp6:133903.Seq | M00001353A:G12 |
| 512 | 14929 | 172.D3.sp6:133915.Seq | M00001353D:D10 |
| 513 | 14391 | 172.H3.sp6:133963.Seq | M00001355B:G10 |
| 514 | 6583 | 172.B8.sp6:133896.Seq | M00001394A:F01 |
| 515 | 4009 | 172.D8.sp6:133920.Seq | M00001396A:C03 |
| 516 | | 172.B9.sp6:133897.Seq | M00001400B:H06 |
| 517 | | 176.A3.sp6:134514.Seq | M00001632D:H07 |
| 518 | 19267 | 176.G3.sp6:134586.Seq | M00001645A:C12 |
| 519 | 78091 | 176.G5.sp6:134588.Seq | M00001679C:F01 |
| 520 | 17055 | 176.D6.sp6:134553.Seq | M00001682C:B12 |
| 521 | 6539 | 176.D9.sp6:134556.Seq | M00003844C:B11 |
| 522 | | 177.H4.sp6:134791.Seq | M00004121B:G01 |
| 523 | 5257 | 177.F5.sp6:134768.Seq | M00004146C:C11 |
| 524 | 11494 | 177.E6.sp6:134757.Seq | M00004172C:D08 |
| 525 | | 177.G7.sp6:134782.Seq | M00004205D:F06 |
| 526 | 11451 | 177.D8.sp6:134747.Seq | M00004214C:H05 |
| 527 | 9283 | 173.D2.SP6:134106.Seq | M00001455D:F09 |
| 528 | 16283 | 173.F3.SP6:134131.Seq | M00001467A:D08 |
| 529 | 10539 | 173.B5.SP6:134085.Seq | M00001499B:A11 |
| 530 | 6420 | 173.F5.SP6:134133.Seq | M00001504D:G06 |
| 531 | 3956 | 173.H5.SP6:134157.Seq | M00001512D:G09 |
| 532 | | 173.G7.SP6:134147.Seq | M00001544A:E06 |
| 533 | 1577 | 173.C9.SP6:134101.Seq | M00001556A:F11 |
| 534 | 9635 | 173.D9.SP6:134113.Seq | M00001557A:F01 |
| 535 | 5192 | 173.E9.SP6:134125.Seq | M00001557B:H10 |
| 536 | 6539 | 173.A12.SP6:134080.Seq | M00001579D:C03 |
| 537 | 945 | 180.C2.sp6:135940.Seq | M00001362C:H11 |
| 538 | 7005 | 180.H5.sp6:136003.Seq | M00001410A:D07 |
| 539 | 39304 | 180.G9.sp6:135995.Seq | M00001450A:A02 |
| 540 | 27250 | 180.B10.sp6:135936.Seq | M00001450A:D08 |
| 541 | 35555 | 184.A5.sp6:135530.Seq | M00001528A:C04 |
| 542 | 19255 | 184.B10.sp6:135547.Seq | M00001545A:C03 |
| 543 | 6268 | 184.C12.sp6:135561.Seq | M00001551A:B10 |
| 544 | 3277 | 217.E1.sp6:139406.Seq | M00001624A:B06 |
| 545 | 39171 | 217.A12.sp6:139369.Seq | M00001644C:B07 |
| 546 | 11460 | 219.F2.sp6:139035.Seq | M00001676B:F05 |
| 547 | 10539 | 219.F6.sp6:139039.Seq | M00001680D:F08 |
| 548 | 11476 | 219.H8.sp6:139065.Seq | M00003747D:C05 |
| 549 | 4016 | 79.A1.sp6:130016.Seq | M00001395A:C03 |
| 550 | 7674 | 79.C1.sp6:130040.Seq | M00001416A:H01 |
| 551 | 3681 | 79.E1.sp6:130064.Seq | M00001449A:D12 |
| 552 | 39304 | 79.F1.sp6:130076.Seq | M00001450A:A02 |
| 553 | 82498 | 79.G1.sp6:130088.Seq | M00001450A:B12 |
| 554 | 84328 | 79.A2.sp6:130017.Seq | M00001452A:B04 |
| 555 | 86859 | 79.B2.sp6:130029.Seq | M00001452A:B12 |
| 556 | 1120 | 79.C2.sp6:130041.Seq | M00001452A:D08 |
| 557 | 85064 | 79.D2.sp6:130053.Seq | M00001452A:F05 |
| 558 | 83103 | 79.G2.sp6:130089.Seq | M00001454A:A09 |
| 559 | 10145 | 79.F3.sp6:130078.Seq | M00001465A:B11 |
| 560 | 16283 | 79.H3.sp6:130102.Seq | M00001467A:D08 |
| 561 | 4568 | 79.D4.sp6:130055.Seq | M00001513A:B06 |
| 562 | 4313 | 79.F4.sp6:130079.Seq | M00001517A:B07 |
| 563 | 2428 | 79.A5.sp6:130020.Seq | M00001533A:C11 |
| 564 | 39423 | 79.C5.sp6:130044.Seq | M00001535A:F10 |
| 565 | 39174 | 79.E5.sp6:130068.Seq | M00001541A:H03 |
| 566 | 22113 | 79.F5.sp6:130080.Seq | M00001542A:A09 |
| 567 | 19829 | 79.H5.sp6:130104.Seq | M00001544A:G02 |
| 568 | 13864 | 79.B6.sp6:130033.Seq | M00001545A:D08 |
| 569 | 1058 | 79.F6.sp6:130081.Seq | M00001548A:H09 |
| 570 | 4015 | 79.G6.sp6:130093.Seq | M00001549A:B02 |
| 571 | 39180 | 79.A7.sp6:130022.Seq | M00001551A:F05 |
| 572 | 307 | 79.C7.sp6:130046.Seq | M00001552A:B12 |
| 573 | 39458 | 79.D7.sp6:130058.Seq | M00001552A:D11 |
| 574 | 39490 | 79.G7.sp6:130094.Seq | M00001557A:F03 |
| 575 | 39486 | 79.B8.sp6:130035.Seq | M00001561A:C05 |
| 576 | 39380 | 79.E8.sp6:130071.Seq | M00001587A:B11 |
| 577 | 1399 | 79.G8.sp6:130095.Seq | M00001604A:B10 |
| 578 | 39391 | 79.A9.sp6:130024.Seq | M00001604A:F05 |
| 579 | 6268 | 79.G9.sp6:130096.Seq | M00001551A:B10 |
| 580 | | 377.F4.sp6:141957.Seq | M00004692A:H08 |
| 581 | 2448 | 89.A1.sp6:130667.Seq | M00001460A:F06 |
| 582 | 1531 | 89.C1.sp6:130691.Seq | M00001461A:D06 |
| 583 | 19 | 89.D1.sp6:130703.Seq | M00001463C:B11 |
| 584 | 38759 | 89.F1.sp6:130727.Seq | M00001467A:B07 |
| 585 | 39508 | 89.G1.sp6:130739.Seq | M00001467A:D04 |
| 586 | 16283 | 89.H1.sp6:130751.Seq | M00001467A:D08 |
| 587 | 39442 | 89.A2.sp6:130668.Seq | M00001467A:E10 |
| 588 | 7589 | 89.B2.sp6:130680.Seq | M00001468A:F05 |
| 589 | | 89.C2.sp6:130692.Seq | M00001469A:A01 |
| 590 | 12081 | 89.D2.sp6:130704.Seq | M00001469A:C10 |
| 591 | 19105 | 89.E2.sp6:130716.Seq | M00001469A:H12 |
| 592 | 1037 | 89.F2.sp6:130728.Seq | M00001470A:B10 |
| 593 | 39425 | 89.G2.sp6:130740.Seq | M00001470A:C04 |
| 594 | 39478 | 89.H2.sp6:130752.Seq | M00001471A:B01 |
| 595 | | 89.B3.sp6:130681.Seq | M00001487B:H06 |
| 596 | | 89.C3.sp6:130693.Seq | M00001488B:F12 |
| 597 | 18699 | 89.D3.sp6:130705.Seq | M00001490B:C04 |
| 598 | 7206 | 89.E3.sp6:130717.Seq | M00001494D:F06 |
| 599 | 2623 | 89.F3.sp6:130729.Seq | M00001497A:G02 |
| 600 | 10539 | 89.G3.sp6:130741.Seq | M00001499B:A11 |
| 601 | 5336 | 89.H3.sp6:130753.Seq | M00001500A:C05 |
| 602 | 2623 | 89.A4.sp6:130670.Seq | M00001500A:E11 |
| 603 | 9443 | 89.B4.sp6:130682.Seq | M00001500C:E04 |
| 604 | 9685 | 89.C4.sp6:130694.Seq | M00001501D:C02 |
| 605 | | 89.D4.sp6:130706.Seq | M00001504A:E01 |
| 606 | 10185 | 89.E4.sp6:130718.Seq | M00001504C:A07 |
| 607 | 6974 | 89.F4.sp6:130730.Seq | M00001504C:H06 |
| 608 | 6420 | 89.G4.sp6:130742.Seq | M00001504D:G06 |
| 609 | | 89.H4.sp6:130754.Seq | M00001505C:C05 |
| 610 | | 89.A5.sp6:130671.Seq | M00001506D:A09 |
| 611 | 39168 | 89.B5.sp6:130683.Seq | M00001507A:H05 |
| 612 | 39412 | 89.C5.sp6:130695.Seq | M00001511A:H06 |
| 613 | 39186 | 89.D5.sp6:130707.Seq | M00001512A:A09 |
| 614 | 3956 | 89.E5.sp6:130719.Seq | M00001512D:G09 |
| 615 | | 89.F5.sp6:130731.Seq | M00001513B:G03 |
| 616 | 14364 | 89.G5.sp6:130743.Seq | M00001513C:E08 |
| 617 | 40044 | 89.H5.sp6:130755.Seq | M00001514C:D11 |
| 618 | 8952 | 89.A6.sp6:130672.Seq | M00001518C:B11 |
| 619 | 35555 | 89.B6.sp6:130684.Seq | M00001528A:C04 |
| 620 | 18957 | 89.C6.sp6:130696.Seq | M00001528A:F09 |
| 621 | 8358 | 89.D6.sp6:130708.Seq | M00001528B:H04 |
| 622 | 38085 | 89.E6.sp6:130720.Seq | M00001531A:D01 |
| 623 | | 89.F6.sp6:130732.Seq | M00001531A:H11 |
| 624 | 3990 | 89.G6.sp6:130744.Seq | M00001532B:A06 |
| 625 | 16921 | 89.H6.sp6:130756.Seq | M00001534A:C04 |
| 626 | 5321 | 89.B7.sp6:130685.Seq | M00001534A:F09 |
| 627 | 4119 | 89.C7.sp6:130697.Seq | M00001534C:A01 |
| 628 | 20212 | 89.E7.sp6:130721.Seq | M00001535A:C06 |
| 629 | 2696 | 89.F7.sp6:130733.Seq | M00001536A:B07 |
| 630 | 39392 | 89.G7.sp6:130745.Seq | M00001536A:C08 |
| 631 | 39420 | 89.H7.sp6:130757.Seq | M00001537A:F12 |
| 632 | 3389 | 89.A8.sp6:130674.Seq | M00001537B:G07 |
| 633 | 8286 | 89.B8.sp6:130686.Seq | M00001540A:D06 |
| 634 | 3765 | 89.C8.sp6:130698.Seq | M00001541A:D02 |
| 635 | 39453 | 89.E8.sp6:130722.Seq | M00001542A:E06 |
| 636 | | 89.F8.sp6:130734.Seq | M00001542B:B01 |
| 637 | | 89.H8.sp6:130758.Seq | M00001544A:E06 |
| 638 | 6974 | 89.A9.sp6:130675.Seq | M00001544B:B07 |
| 639 | | 89.B9.sp6:130687.Seq | M00001545A:B02 |
| 640 | 19255 | 89.C9.sp6:130699.Seq | M00001545A:C03 |
| 641 | 1267 | 89.D9.sp6:130711.Seq | M00001546A:G11 |
| 642 | 5892 | 89.E9.sp6:130723.Seq | M00001548A:E10 |
| 643 | 4193 | 89.G9.sp6:130747.Seq | M00001549B:F06 |
| 644 | 16347 | 89.H9.sp6:130759.Seq | M00001549C:E06 |
| 645 | 7239 | 89.A10.sp6:130676.Seq | M00001550A:A03 |
| 646 | 5175 | 89.B10.sp6:130688.Seq | M00001550A:G01 |
| 647 | 22390 | 89.C10.sp6:130700.Seq | M00001551A:G06 |
| 648 | 3266 | 89.D10.sp6:130712.Seq | M00001551C:G09 |
| 649 | 5708 | 89.E10.sp6:130724.Seq | M00001552B:D04 |
| 650 | | 89.F10.sp6:130736.Seq | M00001552D:A01 |
| 651 | 8298 | 89.G10.sp6:130748.Seq | M00001553A:H06 |
| 652 | 4573 | 89.H10.sp6:130760.Seq | M00001553B:F12 |
| 653 | 22814 | 89.A11.sp6:130677.Seq | M00001553D:D10 |
| 654 | 39539 | 89.B11.sp6:130689.Seq | M00001555A:B02 |
| 655 | 39195 | 89.C11.sp6:130701.Seq | M00001555A:C01 |
| 656 | 4561 | 89.D11.sp6:130713.Seq | M00001555D:G10 |
| 657 | 9244 | 89.E11.sp6:130725.Seq | M00001556A:C09 |
| 658 | 1577 | 89.F11.sp6:130737.Seq | M00001556A:F11 |
| 659 | 4386 | 89.H11.sp6:130761.Seq | M00001556B:C08 |
| 660 | 11294 | 89.A12.sp6:130678.Seq | M00001556B:G02 |
| 661 | 5192 | 89.D12.sp6:130714.Seq | M00001557B:H10 |
| 662 | 8761 | 89.E12.sp6:130726.Seq | M00001557D:D09 |
| 663 | | 89.F12.sp6:130738.Seq | M00001558A:H05 |
| 664 | 7514 | 89.G12.sp6:130750.Seq | M00001558B:H11 |
| 665 | | 89.H12.sp6:130762.Seq | M00001559B:F01 |
| 666 | 6558 | 90.A1.sp6:130859.Seq | M00001560D:F10 |
| 667 | 102 | 90.B1.sp6:130871.Seq | M00001563B:F06 |
| 668 | | 90.D1.sp6:130895.Seq | M00001566B:D11 |
| 669 | 5749 | 90.E1.sp6:130907.Seq | M00001571C:H06 |
| 670 | 6539 | 90.G1.sp6:130931.Seq | M00001579D:C03 |
| 671 | 6293 | 90.A2.sp6:130860.Seq | M00001583D:A10 |
| 672 | | 90.C2.sp6:130884.Seq | M00001590B:F03 |
| 673 | 260 | 90.D2.sp6:130896.Seq | M00001594B:H04 |
| 674 | 4837 | 90.E2.sp6:130908.Seq | M00001597C:H02 |
| 675 | 10470 | 90.F2.sp6:130920.Seq | M00001597D:C05 |
| 676 | 16999 | 90.G2.sp6:130932.Seq | M00001598A:G03 |
| 677 | 22794 | 90.H2.sp6:130944.Seq | M00001601A:D08 |
| 678 | 11465 | 90.A3.sp6:130861.Seq | M00001607A:E11 |
| 679 | 7802 | 90.B3.sp6:130873.Seq | M00001608A:B03 |
| 680 | 22155 | 90.C3.sp6:130885.Seq | M00001608B:E03 |
| 681 | | 90.D3.sp6:130897.Seq | M00001608D:A11 |
| 682 | 13157 | 90.E3.sp6:130909.Seq | M00001614C:F10 |
| 683 | 17004 | 90.F3.sp6:130921.Seq | M00001617C:E02 |
| 684 | 40314 | 90.G3.sp6:130933.Seq | M00001619C:F12 |
| 685 | 40044 | 90.H3.sp6:130945.Seq | M00001621C:C08 |
| 686 | 13913 | 90.A4.sp6:130862.Seq | M00001623D:F10 |
| 687 | 3277 | 90.B4.sp6:130874.Seq | M00001624A:B06 |
| 688 | 4309 | 90.C4.sp6:130886.Seq | M00001624C:F01 |
| 689 | 5214 | 90.D4.sp6:130898.Seq | M00001630B:H09 |
| 690 | | 90.E4.sp6:130910.Seq | M00001632D:H07 |
| 691 | 39171 | 90.F4.sp6:130922.Seq | M00001644C:B07 |
| 692 | 19267 | 90.G4.sp6:130934.Seq | M00001645A:C12 |
| 693 | 4665 | 90.H4.sp6:130946.Seq | M00001648C:A01 |
| 694 | | 90.A5.sp6:130863.Seq | M00001651A:H01 |
| 695 | 23201 | 90.B5.sp6:130875.Seq | M00001657D:C03 |
| 696 | 76760 | 90.C5.sp6:130887.Seq | M00001657D:F08 |
| 697 | 23218 | 90.D5.sp6:130899.Seq | M00001662C:A09 |
| 698 | 35702 | 90.E5.sp6:130911.Seq | M00001663A:E04 |
| 699 | 6468 | 90.F5.sp6:130923.Seq | M00001669B:F02 |
| 700 | 14367 | 90.G5.sp6:130935.Seq | M00001670C:H02 |
| 701 | 7015 | 90.H5.sp6:130947.Seq | M00001673C:H02 |
| 702 | 8773 | 90.A6.sp6:130864.Seq | M00001675A:C09 |
| 703 | 11460 | 90.B6.sp6:130876.Seq | M00001676B:F05 |
| 704 | 7570 | 90.D6.sp6:130900.Seq | M00001677D:A07 |
| 705 | 4416 | 90.E6.sp6:130912.Seq | M00001678D:F12 |
| 706 | 6660 | 90.F6.sp6:130924.Seq | M00001679A:A06 |
| 707 | | 90.H6.sp6:130948.Seq | M00001679A:F06 |
| 708 | 26875 | 90.A7.sp6:130865.Seq | M00001679A:F10 |
| 709 | 6298 | 90.B7.sp6:130877.Seq | M00001679B:F01 |
| 710 | 78091 | 90.C7.sp6:130889.Seq | M00001679C:F01 |
| 711 | 10751 | 90.D7.sp6:130901.Seq | M00001679D:D03 |
| 712 | 10539 | 90.F7.sp6:130925.Seq | M00001680D:F08 |
| 713 | 17055 | 90.G7.sp6:130937.Seq | M00001682C:B12 |
| 714 | 5382 | 90.A8.sp6:130866.Seq | M00001688C:F09 |
| 715 | 4393 | 90.B8.sp6:130878.Seq | M00001693C:G01 |
| 716 | 67252 | 90.C8.sp6:130890.Seq | M00001716D:H05 |
| 717 | 40108 | 90.D8.sp6:130902.Seq | M00003741D:C09 |
| 718 | 11476 | 90.E8.sp6:130914.Seq | M00003747D:C05 |
| 719 | | 90.F8.sp6:130926.Seq | M00003754C:E09 |
| 720 | 697 | 90.G8.sp6:130938.Seq | M00003759B:B09 |
| 721 | | 90.H8.sp6:130950.Seq | M00003761D:A09 |
| 722 | 17076 | 90.A9.sp6:130867.Seq | M00003762C:B08 |
| 723 | 3108 | 90.B9.sp6:130879.Seq | M00003763A:F06 |
| 724 | 67907 | 90.C9.sp6:130891.Seq | M00003774C:A03 |
| 725 | | 90.D9.sp6:130903.Seq | M00003784D:D12 |
| 726 | 11350 | 90.F9.sp6:130927.Seq | M00003826B:A06 |
| 727 | 7899 | 90.H9.sp6:130951.Seq | M00003837D:A01 |
| 728 | 7798 | 90.A10.sp6:130868.Seq | M00003839A:D08 |
| 729 | 6539 | 90.B10.sp6:130880.Seq | M00003844C:B11 |
| 730 | 6874 | 90.C10.sp6:130892.Seq | M00003846B:D06 |
| 731 | | 90.D10.sp6:130904.Seq | M00003851B:D08 |
| 732 | 13595 | 90.E10.sp6:130916.Seq | M00003851B:D10 |
| 733 | 5619 | 90.F10.sp6:130928.Seq | M00003853A:D04 |
| 734 | 10515 | 90.G10.sp6:130940.Seq | M00003853A:F12 |
| 735 | 4622 | 90.H10.sp6:130952.Seq | M00003856B:C02 |
| 736 | 3389 | 90.A11.sp6:130869.Seq | M00003857A:G10 |
| 737 | 4718 | 90.B11.sp6:130881.Seq | M00003857A:H03 |
| 738 | | 90.C11.sp6:130893.Seq | M00003867A:D10 |
| 739 | 12977 | 90.F11.sp6:130929.Seq | M00003875B:F04 |
| 740 | 8479 | 90.G11.sp6:130941.Seq | M00003875C:G07 |
| 741 | | 90.H11.sp6:130953.Seq | M00003875D:D11 |
| 742 | 7798 | 90.A12.sp6:130870.Seq | M00003876D:E12 |
| 743 | 5345 | 90.B12.sp6:130882.Seq | M00003879B:C11 |
| 744 | 31587 | 90.C12.sp6:130894.Seq | M00003879B:D10 |
| 745 | 14507 | 90.D12.sp6:130906.Seq | M00003879D:A02 |
| 746 | 13576 | 90.F12.sp6:130930.Seq | M00003885C:A02 |
| 747 | | 90.G12.sp6:130942.Seq | M00003891C:H09 |
| 748 | 9285 | 90.H12.sp6:130954.Seq | M00003906C:E10 |
| 749 | 39809 | 99.A1.sp6:131230.Seq | M00003907D:A09 |
| 750 | 16317 | 99.B1.sp6:131242.Seq | M00003907D:H04 |
| 751 | 8672 | 99.C1.sp6:131254.Seq | M00003909D:C03 |
| 752 | 12532 | 99.D1.sp6:131266.Seq | M00003912B:D01 |
| 753 | 3900 | 99.E1.sp6:131278.Seq | M00003914C:F05 |
| 754 | 23255 | 99.F1.sp6:131290.Seq | M00003922A:E06 |
| 755 | 24488 | 99.C2.sp6:131255.Seq | M00003968B:F06 |
| 756 | 40122 | 99.D2.sp6:131267.Seq | M00003970C:B09 |
| 757 | 23210 | 99.E2.sp6:131279.Seq | M00003974D:E07 |
| 758 | 23358 | 99.F2.sp6:131291.Seq | M00003974D:H02 |
| 759 | 3430 | 99.A3.sp6:131232.Seq | M00003981A:E10 |
| 760 | 2433 | 99.B3.sp6:131244.Seq | M00003982C:C02 |
| 761 | 9105 | 99.C3.sp6:131256.Seq | M00003983A:A05 |
| 762 | 6124 | 99.D3.sp6:131268.Seq | M00004028D:A06 |
| 763 | 40073 | 99.E3.sp6:131280.Seq | M00004028D:C05 |
| 764 | 37285 | 99.H3.sp6:131316.Seq | M00004035C:A07 |
| 765 | 17036 | 99.A4.sp6:131233.Seq | M00004035D:B06 |
| 766 | 3706 | 99.C4.sp6:131257.Seq | M00004068B:A01 |
| 767 | | 99.D4.sp6:131269.Seq | M00004072A:C03 |
| 768 | 15069 | 99.F4.sp6:131293.Seq | M00004081C:D10 |
| 769 | 9285 | 99.H4.sp6:131317.Seq | M00004086D:G06 |
| 770 | 6880 | 99.A5.sp6:131234.Seq | M00004087D:A01 |
| 771 | 5325 | 99.C5.sp6:131258.Seq | M00004093D:B12 |
| 772 | 7221 | 99.D5.sp6:131270.Seq | M00004105C:A04 |
| 773 | 4937 | 99.E5.sp6:131282.Seq | M00004108A:E06 |
| 774 | 6874 | 99.F5.sp6:131294.Seq | M00004111D:A08 |
| 775 | 13183 | 99.G5.sp6:131306.Seq | M00004114C:F11 |
| 776 | | 99.H5.sp6:131318.Seq | M00004121B:G01 |
| 777 | 13272 | 99.A6.sp6:131235.Seq | M00004138B:H02 |
| 778 | 5257 | 99.B6.sp6:131247.Seq | M00004146C:C11 |
| 779 | 6455 | 99.D6.sp6:131271.Seq | M00004157C:A09 |
| 780 | 5319 | 99.E6.sp6:131283.Seq | M00004169C:C12 |
| 781 | 4908 | 99.F6.sp6:131295.Seq | M00004171D:B03 |
| 782 | 11494 | 99.G6.sp6:131307.Seq | M00004172C:D08 |
| 783 | 11443 | 99.A7.sp6:131236.Seq | M00004185C:C03 |
| 784 | | 99.B7.sp6:131248.Seq | M00004191D:B11 |
| 785 | 8210 | 99.C7.sp6:131260.Seq | M00004197D:H01 |
| 786 | 14311 | 99.D7.sp6:131272.Seq | M00004203B:C12 |
| 787 | | 99.E7.sp6:131284.Seq | M00004205D:F06 |
| 788 | 12971 | 99.B8.sp6:131249.Seq | M00004223D:E04 |
| 789 | 6455 | 99.C8.sp6:131261.Seq | M00004229B:F08 |
| 790 | 7212 | 99.D8.sp6:131273.Seq | M00004230B:C07 |
| 791 | 4905 | 99.H8.sp6:131321.Seq | M00004269D:D06 |
| 792 | 16914 | 99.A9.sp6:131238.Seq | M00004275C:C11 |
| 793 | 16921 | 99.D9.sp6:131274.Seq | M00004295D:F12 |
| 794 | 13046 | 99.E9.sp6:131286.Seq | M00004296C:H07 |
| 795 | 9457 | 99.F9.sp6:131298.Seq | M00004307C:A06 |
| 796 | 26295 | 99.G9.sp6:131310.Seq | M00004312A:G03 |
| 797 | 21847 | 99.H9.sp6:131322.Seq | M00004318C:D10 |
| 798 | | 99.H10.sp6:131323.Seq | M00004505D:F08 |
| 799 | | 99.B11.sp6:131252.Seq | M00004692A:H08 |
| 800 | | 99.D11.sp6:131276.Seq | M00005180C:G03 |
| 801 | 39304 | RTA00000118A.j.21.1.Seq_THC151859 | |
| 802 | 2428 | RTA00000123A.l.21.1.Seq_THC205063 | |
| 803 | 1058 | RTA00000126A.e.20.3.Seq_THC217534 | |
| 804 | 5097 | RTA00000134A.k.1.1.Seq_THC215869 | |
| 805 | 20212 | RTA00000134A.l.22.1.Seq_THC128232 | |
| 806 | 23255 | RTA00000177AF.e.14.3.Seq_THC228776 | |
| 807 | 2790 | RTA00000177AF.e.2.1.Seq_THC229461 | |
| 808 | 6420 | RTA00000177AF.f.10.3.Seq_THC226443 | |
| 809 | 4059 | RTA00000177AF.n.18.3.Seq_THC123051 | |
| 810 | | RTA00000179AF.j.13.1.Seq_THC105720 | |
| 811 | 9952 | RTA00000180AF.c.20.1.Seq_THC162284 | |
| 812 | 13238 | RTA00000181AF.m.4.1.Seq_THC140691 | |
| 813 | 9685 | RTA00000183AF.c.11.1.Seq_THC109544 | |
| 814 | | RTA00000183AF.c.24.1.Seq_THC125912 | |
| 815 | 6420 | RTA00000183AF.d.11.1.Seq_THC226443 | |
| 816 | 6974 | RTA00000183AF.d.9.1.Seq_THC223129 | |
| 817 | 40044 | RTA00000183AF.g.22.1.Seq_THC232899 | |
| 818 | | RTA00000183AF.g.9.1.Seq_THC198280 | |
| 819 | 5892 | RTA00000184AF.d.11.1.Seq_THC161896 | |
| 820 | 40044 | RTA00000186AF.d.1.1.Seq_THC232899 | |
| 821 | | RTA00000186AF.h.14.1.Seq_THC112525 | |
| 822 | 19267 | RTA00000186AF.l.12.1.Seq_THC178183 | |
| 823 | 8773 | RTA00000187AF.f.24.1.Seq_THC220002 | |
| 824 | 7570 | RTA00000187AF.g.24.1.Seq_THC168636 | |
| 825 | 11476 | RTA00000187AF.p.19.1.Seq_THC108482 | |
| 826 | | RTA00000188AF.d.11.1.Seq_THC212094 | |
| 827 | 17076 | RTA00000188AF.d.21.1.Seq_THC208760 | |
| 828 | 697 | RTA00000188AF.d.6.1.Seq_THC178884 | |
| 829 | 67907 | RTA00000188AF.g.11.1.Seq_THC123222 | |
| 830 | 5619 | RTA00000188AF.l.9.1.Seq_THC167845 | |
| 831 | 4718 | RTA00000189AF.g.5.1.Seq_THC196102 | |
| 832 | 39809 | RTA00000190AF.e.3.1.Seq_THC150217 | |
| 833 | 23255 | RTA00000190AF.j.4.1.Seq_THC228776 | |
| 834 | 40122 | RTA00000190AF.n.23.1.Seq_THC109227 | |
| 835 | 23210 | RTA00000190AF.o.20.1.Seq_THC207240 | |
| 836 | 23358 | RTA00000190AF.o.21.1.Seq_THC207240 | |
| 837 | 5693 | RTA00000190AF.p.17.2.Seq_THC173318 | |
| 838 | 2433 | RTA00000191AF.a.15.2.Seq_THC79498 | |
| 839 | 5257 | RTA00000192AF.f.3.1.Seq_THC213833 | |
| 840 | 16392 | RTA00000192AF.l.1.1.Seq_THC202071 | |
| 841 | | RTA00000193AF.c.21.1.Seq_THC222602 | |
| 842 | 26295 | RTA00000193AF.i.24.2.Seq_THC197345 | |
| 843 | | RTA00000193AF.m.5.1.Seq_THC173318 | |
| 844 | | RTA00000193AF.n.15.1.Seq_THC215687 | |

TABLE 2

| | | | | Nearest | | |
|---|---|---|---|---|---|---|
| | | | | Neighbor | | |
| | Nearest | | | (BlastX vs. | | |
| | Neighbor | | | Non- | | |
| | (BlastN vs. | | | Redundant | | |
| SEQ | Genbank) | | P | Proteins) | | P |
| ID | ACCESSION | DESCRIPTION | VALUE | ACCESSION | DESCRIPTION | VALUE |
| 1 | <NONE> | <NONE> | <NONE> | <NONE> | <NONE> | <NONE> |
| 2 | <NONE> | <NONE> | <NONE> | <NONE> | <NONE> | <NONE> |
| 3 | <NONE> | <NONE> | <NONE> | <NONE> | <NONE> | <NONE> |
| 4 | <NONE> | <NONE> | <NONE> | BAR3_CHITE | BALBIANI RING | 1 |
| | | | | | PROTEIN 3 | |
| | | | | | PRECURSOR>PIR2:S08 | |
| | | | | | 167 Balbiani ring 3 | |
| | | | | | protein - midge | |
| | | | | | (Chironomus | |
| | | | | | tentans)>GP:CTBR3_1 | |
| | | | | | C;tentans balbiani ring 3 | |
| | | | | | (BR3) gene | |
| 5 | <NONE> | <NONE> | <NONE> | CYAA_PODAN | ADENYLATE | 1 |
| | | | | | CYCLASE (EC 4.6.1.1) | |
| | | | | | (ATP | |
| | | | | | PYROPHOSPHATE- | |
| | | | | | LYASE) (ADENYLYL | |
| | | | | | CYCLASE)>PIR2:JC47 | |
| | | | | | 47 adenylate cyclase (EC | |
| | | | | | 4.6.1.1) - Podospora | |
| | | | | | anserina>GP:PANADCY_ | |
| | | | | | 1 Podospora anserina | |
| | | | | | adenyl cyclase gene, | |
| | | | | | exons 1-4 | |
| 6 | <NONE> | <NONE> | <NONE> | VP03_HSVSA | PROBABLE | 0.97 |
| | | | | | MEMBRANE | |
| | | | | | ANTIGEN 3 | |
| | | | | | (TEGUMENT | |
| | | | | | PROTEIN)>PIR2:C3680 | |
| | | | | | 6 hypothetical protein | |
| | | | | | ORF3 - saimiriine | |
| | | | | | herpesvirus 1 (strain | |
| | | | | | 11)>GP:HSGEND_3 | |
| | | | | | Herpesvirus saimiri | |
| | | | | | complete genome DNA; | |
| | | | | | ORF 03; similarity to | |
| | | | | | ORF 75 and EBV | |
| | | | | | BNRF1 | |
| 7 | <NONE> | <NONE> | <NONE> | ATFCA2_18 | Arabidopsis thaliana | 0.93 |
| | | | | | DNA chromosome 4, | |
| | | | | | ESSA I contig fragment | |
| | | | | | No; 2; Hydroxyproline- | |
| | | | | | rich glycoprotein | |
| | | | | | homolog; Similarity to | |
| | | | | | hydroxyproline-rich | |
| | | | | | glycoprotein precursor- | |
| | | | | | common tobacco | |
| 8 | <NONE> | <NONE> | <NONE> | DHAL_ASPNG | ALDEHYDE | 0.9 |
| | | | | | DEHYDROGENASE | |
| | | | | | (EC 1.2.1.3) | |
| | | | | | (ALDDH)>GP:ASNALD | |
| | | | | | AA_1 Aspergillus niger | |
| | | | | | aldehyde dehydrogenase | |
| | | | | | (aldA) gene, complete | |
| | | | | | cds | |
| 9 | <NONE> | <NONE> | <NONE> | NCU50264_1 | Neurospora crassa two- | 0.86 |
| | | | | | component histidine | |
| | | | | | kinase (nik-1) gene, 5′ | |
| | | | | | region and partial cds | |
| 10 | <NONE> | <NONE> | <NONE> | NEUG_BOVIN | NEUROGRANIN (P17) | 0.82 |
| | | | | | (B-50 | |
| | | | | | IMMUNOREACTIVE | |
| | | | | | C-KINASE | |
| | | | | | SUBSTRATE) (BICKS) | |
| | | | | | (FRAGMENT)>PIR2:A3 | |
| | | | | | 9034 neurogranin - | |
| | | | | | bovine (fragment) | |
| 11 | <NONE> | <NONE> | <NONE> | HUMBYSTIN_1 | Homo sapiens bystin | 0.81 |
| | | | | | mRNA, complete cds | |
| 12 | <NONE> | <NONE> | <NONE> | BTBMP1_1 | Bos taurus BMP1 gene, | 0.69 |
| | | | | | partial sequence; Bone | |
| | | | | | morphogenetic protein 1 | |
| 13 | <NONE> | <NONE> | <NONE> | TCCYSPROT_1 | T;congolense mRNA for | 0.56 |
| | | | | | (prepro) cysteine | |
| | | | | | proteinase | |
| 14 | <NONE> | <NONE> | <NONE> | P60_LISIV | PROTEIN P60 | 0.15 |
| | | | | | PRECURSOR | |
| | | | | | (INVASION- | |
| | | | | | ASSOCIATED | |
| | | | | | PROTEIN)>GP:LISIAP | |
| | | | | | RELB_1 Listeria | |
| | | | | | ivanovii extracellular | |
| | | | | | protein homologue (iap) | |
| | | | | | gene, complete cds | |
| 15 | <NONE> | <NONE> | <NONE> | HEX_ADE31 | HEXON PROTEIN | 0.15 |
| | | | | | (LATE PROTEIN 2) | |
| | | | | | (FRAGMENT)>PIR2:S3 | |
| | | | | | 7217 hexon protein - | |
| | | | | | human adenovirus 31 | |
| | | | | | (fragment)>GP:HSAT31 | |
| | | | | | H_1 H; sapiens | |
| | | | | | adenovirus type 31 hexon | |
| | | | | | gene; Hexon protein; | |
| | | | | | Internal fragment | |
| | | | | | containing hypervariable | |
| | | | | | regions | |
| 16 | <NONE> | <NONE> | <NONE> | HSU77493_1 | Human Notch2 mRNA, | 0.13 |
| | | | | | partial cds; | |
| | | | | | Transmembrane protein; | |
| | | | | | hN | |
| 17 | <NONE> | <NONE> | <NONE> | CYB_PARTE | CYTOCHROME B (EC | 0.078 |
| | | | | | 1.10.2.2)>PIR2:S07743 | |
| | | | | | cytochrome b - | |
| | | | | | Paramecium tetraurelia | |
| | | | | | mitochondrion | |
| | | | | | (SGC6)>GP:MIPAGEN_{—} | |
| | | | | | 19 Paramecium aurelia | |
| | | | | | mitochondrial complete | |
| | | | | | genome; Apocytochrome | |
| | | | | | b (AA 1-391) | |
| 18 | <NONE> | <NONE> | <NONE> | HUMERB27_1 | Human c-erbB-2 gene, | 0.054 |
| | | | | | exon 7; C-erb-2 protein | |
| 19 | <NONE> | <NONE> | <NONE> | DMTRXIII_2 | D; melanogaster DNA for | 0.047 |
| | | | | | trxI and trxII genes; | |
| | | | | | Trithorax protein trxI; | |
| | | | | | Trithorax; | |
| | | | | | putative>GP:DMTTHOR | |
| | | | | | AX_2 D; melanogaster | |
| | | | | | DNA for (putative) | |
| | | | | | trithorax protein; | |
| | | | | | Predicted trithorax | |
| | | | | | protein | |
| 20 | <NONE> | <NONE> | <NONE> | CELB0281_5 | Caenorhabditis elegans | 0.043 |
| | | | | | cosmid B0281; Similar to | |
| | | | | | reverse transcriptases | |
| 21 | <NONE> | <NONE> | <NONE> | MOTY_VIBPA | SODIUM-TYPE | 0.041 |
| | | | | | FLAGELLAR PROTEIN | |
| | | | | | MOTY | |
| | | | | | PRECURSOR>GP:VPU | |
| | | | | | 06949_4 Vibrio | |
| | | | | | parahaemolyticus BB22 | |
| | | | | | RNase T (rnt) gene and | |
| | | | | | flagellar motor | |
| | | | | | component (motY) gene, | |
| | | | | | complete cds | |
| 22 | <NONE> | <NONE> | <NONE> | A56263 | beta-galactosidase (EC | 0.04 |
| | | | | | 3.2.1.23) isozyme 12 - | |
| | | | | | Arthrobacter sp. (strain | |
| | | | | | B7)>GP:ASU17417_1 | |
| | | | | | Arthrobacter sp; beta- | |
| | | | | | galactosidase gene, | |
| | | | | | complete cds | |
| 23 | <NONE> | <NONE> | <NONE> | GSA_PSEAE | GLUTAMATE-1- | 0.038 |
| | | | | | SEMIALDEHYDE 2,1- | |
| | | | | | AMINOMUTASE (EC | |
| | | | | | 5.4.3.8) (GSA) | |
| | | | | | (GLUTAMATE-1- | |
| | | | | | SEMIALDEHYDE | |
| | | | | | AMINOTRANSFERAS | |
| | | | | | E) (GSA- | |
| | | | | | AT)>PIR2:S57898 | |
| | | | | | glutamate 1- | |
| | | | | | semialdehyde 2,1- | |
| | | | | | aminomutase - | |
| | | | | | Pseudomonas | |
| | | | | | aeruginosa>GP:PAHEM | |
| | | | | | L_1 P; aeruginosa hemL | |
| | | | | | gene; Glutamate 1-sem | |
| 24 | <NONE> | <NONE> | <NONE> | S16323 | hypothetical protein - | 0.035 |
| | | | | | Arabidopsis | |
| | | | | | thalian>GP:ATHB1_1 | |
| | | | | | A; thalian homeobox | |
| | | | | | gene Athb-1 mRNA; | |
| | | | | | Open reading frame | |
| 25 | <NONE> | <NONE> | <NONE> | IRS1_RAT | INSULIN RECEPTOR | 0.027 |
| | | | | | SUBSTRATE- | |
| | | | | | 1>PIR2:S16948 | |
| | | | | | hypothetical protein IRS- | |
| | | | | | 1 - | |
| | | | | | rat>GP:RNIRS1IRM_1 | |
| | | | | | R; Norvegicus IRS-1 | |
| | | | | | mRNA for insulin- | |
| | | | | | receptor; During insulin | |
| | | | | | stimulation, undergoes | |
| | | | | | tyrosine phosphorylation | |
| | | | | | and binds | |
| | | | | | phosphatidylinositol 3- | |
| | | | | | kinase | |
| 26 | <NONE> | <NONE> | <NONE> | CEM02G9_2 | Caenorhabditis elegans | 0.0088 |
| | | | | | cosmid M02G9; | |
| | | | | | M02G9; 1; Similar to | |
| | | | | | keratin like protein; | |
| | | | | | cDNA EST yk308g11; 5 | |
| | | | | | comes from this gene; | |
| | | | | | cDNA EST yk208e11; 5 | |
| | | | | | comes from this gene; | |
| | | | | | cDNA EST yk208e11; 3 | |
| | | | | | comes | |
| 27 | <NONE> | <NONE> | <NONE> | S75490_3 | competence region: | 0.0041 |
| | | | | | iga=IgA protease, | |
| | | | | | comA=transformation | |
| | | | | | competence [Neisseria | |
| | | | | | gonorrhoeae, MS11, | |
| | | | | | Genomic, 3 genes, 2664 | |
| | | | | | nt] | |
| 28 | <NONE> | <NONE> | <NONE> | EXTN_TOBAC | EXTENSIN | 0.0025 |
| | | | | | PRECURSOR (CELL | |
| | | | | | WALL | |
| | | | | | HYDROXYPROLINE- | |
| | | | | | RICH | |
| | | | | | GLYCOPROTEIN)>PIR | |
| | | | | | 2:S06733 | |
| | | | | | hydroxyproline-rich | |
| | | | | | glycoprotein precursor - | |
| | | | | | common | |
| | | | | | tobacco>GP:NTEXT_1 | |
| | | | | | Tobacco HRGPnt3 gene | |
| | | | | | for extensin; Extensin | |
| | | | | | (AA 1-620) | |
| 29 | <NONE> | <NONE> | <NONE> | HPCEGS_1 | Hepatitis C virus | 0.0014 |
| | | | | | complete genome | |
| | | | | | sequence; Polyprotein | |
| 30 | <NONE> | <NONE> | <NONE> | HHVBC_4 | Human hepatitis virus | 0.00093 |
| | | | | | (genotype C, HMA) | |
| | | | | | preS1, preS2, S, C, X, | |
| | | | | | antigens, core antigen, X | |
| | | | | | protein and polymerase | |
| 31 | <NONE> | <NONE> | <NONE> | HSLTGFBP4_1 | Homo sapiens mRNA for | 0.00061 |
| | | | | | latent transforming | |
| | | | | | growth factor-beta | |
| | | | | | binding protein-4; Latent | |
| | | | | | TGF-beta binding | |
| | | | | | protein-4 | |
| 32 | <NONE> | <NONE> | <NONE> | S74909 | transposase - | 0.00051 |
| | | | | | Synechocystis sp. (PCC | |
| | | | | | 6803)>GP:D90909_108 | |
| | | | | | Synechocystis sp; | |
| | | | | | PCC6803 complete | |
| | | | | | genome, 11/27, 1311235- | |
| | | | | | 1430418; Transposase; | |
| | | | | | ORF_ID:slr2062 | |
| 33 | <NONE> | <NONE> | <NONE> | GRN_MOUSE | GRANULINS | 0.00022 |
| | | | | | PRECURSOR | |
| | | | | | (ACROGRANIN)>GP:M | |
| | | | | | USAP_1 Mouse gene for | |
| | | | | | acrogranin precursor, | |
| | | | | | complete cds | |
| 34 | <NONE> | <NONE> | <NONE> | CA21_MOUSE | PROCOLLAGEN | 0.00016 |
| | | | | | ALPHA 2(I) CHAIN | |
| | | | | | PRECURSOR>PIR2:A4 | |
| | | | | | 3291 collagen alpha 2(I) | |
| | | | | | chain precursor - | |
| | | | | | mouse>GP:MMCOL1A2 | |
| | | | | | _1 Mouse COL1A2 | |
| | | | | | mRNA for pro-alpha-2(I) | |
| | | | | | collagen | |
| 35 | <NONE> | <NONE> | <NONE> | MMMHC29N | Mus musculus major | 8.00E−05 |
| | | | | 7_2 | histocompatibility locus | |
| | | | | | class III | |
| | | | | | region:butyrophilin-like | |
| | | | | | protein gene, partial cds; | |
| | | | | | Notch4, PBX2, RAGE, | |
| | | | | | lysophatidic acid acyl | |
| | | | | | transferase-alpha, | |
| | | | | | palmitoyl- | |
| 36 | <NONE> | <NONE> | <NONE> | NFH_RAT | NEUROFILAMENT | 2.40E−05 |
| | | | | | TRIPLET H PROTEIN | |
| | | | | | (200 KD | |
| | | | | | NEUROFILAMENT | |
| | | | | | PROTEIN) (NF-H) | |
| | | | | | (FRAGMENT) | |
| 37 | <NONE> | <NONE> | <NONE> | HUMVWFM_1 | Human von Willebrand | 1.70E−05 |
| | | | | | factor mRNA, 3′ end; | |
| | | | | | Von Willebrand factor | |
| | | | | | prepropeptide | |
| 38 | <NONE> | <NONE> | <NONE> | CGHU2E | collagen alpha 2(XI) | 2.00E−06 |
| | | | | | chain - human (fragment) | |
| 39 | <NONE> | <NONE> | <NONE> | A61183 | hypothetical protein | 4.90E−08 |
| | | | | | (sdsB region) - | |
| | | | | | Pseudomonas sp. | |
| 40 | <NONE> | <NONE> | <NONE> | YM8L_YEAST | HYPOTHETICAL 71.1 | 1.50E−09 |
| | | | | | KD PROTEIN IN DSK2- | |
| | | | | | CAT8 INTERGENIC | |
| | | | | | REGION>PIR2:S54585 | |
| | | | | | hypothetical protein | |
| | | | | | YMR278w - yeast | |
| | | | | | (Saccharomyces | |
| | | | | | cerevisiae)>GP:SC8021 | |
| | | | | | X_4 S; cerevisiae | |
| | | | | | chromosome XIII cosmid | |
| | | | | | 8021; Unknown; | |
| | | | | | YM8021; 04, unknown, | |
| | | | | | len: 622, CAI: 0; 16, | |
| 41 | <NONE> | <NONE> | <NONE> | MTCY210_31 | Mycobacterium | 3.10E−10 |
| | | | | | tuberculosis cosmid | |
| | | | | | Y210; Unknown; | |
| | | | | | MTCY210; 31, unknown, | |
| | | | | | len: 299 aa, slight | |
| | | | | | similarity to | |
| | | | | | carboxykinases | |
| 42 | <NONE> | <NONE> | <NONE> | CEC01G10_5 | Caenorhabditis elegans | 2.30E−12 |
| | | | | | cosmid C01G10, | |
| | | | | | complete sequence; | |
| | | | | | C01G10; 8; CDNA EST | |
| | | | | | CEMSC45R comes from | |
| | | | | | this | |
| | | | | | gene>GP:CEC01G10_5 | |
| | | | | | Caenorhabditis elegans | |
| | | | | | cosmid C01G10; | |
| | | | | | C01G10; 8; CDNA EST | |
| | | | | | CEMSC45R comes from | |
| | | | | | this gene | |
| 43 | <NONE> | <NONE> | <NONE> | HSU15779_1 | Human p70 (ST5) | 9.50E−14 |
| | | | | | mRNA, alternatively | |
| | | | | | spliced, complete cds; | |
| | | | | | Differentially expressed; | |
| | | | | | alternatively spliced | |
| 44 | <NONE> | <NONE> | <NONE> | MTCY210_31 | Mycobacterium | 1.70E−17 |
| | | | | | tuberculosis cosmid | |
| | | | | | Y210; Unknown; | |
| | | | | | MTCY210; 31, unknown, | |
| | | | | | len: 299 aa, slight | |
| | | | | | similarity to | |
| | | | | | carboxykinases | |
| 45 | U61403 | Dictyostelium | 1 | U93472_1 | Danio rerio PPARB | 0.95 |
| | | discoideum PrlA | | | gene, partial cds; Nuclear | |
| | | (prlA) mRNA, | | | receptor C domain | |
| | | partial cds. | | | | |
| 46 | Z92832 | Caenorhabditis | 1 | U93472_1 | Danio rerio PPARB | 0.94 |
| | | elegans DNA *** | | | gene, partial cds; Nuclear | |
| | | SEQUENCING | | | receptor C domain | |
| | | IN PROGRESS | | | | |
| | | *** from clone | | | | |
| | | F31D4; HTGS | | | | |
| | | phase 1. | | | | |
| 47 | L36557 | Oryza sativa | 1 | HSU61262_1 | Human neogenin mRNA, | 0.89 |
| | | (clone pRG3) | | | complete cds | |
| | | repetitive | | | | |
| | | element. | | | | |
| 48 | AF005898 | Homo sapiens | 1 | LRP1_CHICK | LOW-DENSITY | 0.85 |
| | | Na, K-ATPase | | | LIPOPROTEIN | |
| | | beta-3 subunit | | | RECEPTOR-RELATED | |
| | | pseudogene, | | | PROTEIN 1 | |
| | | complete | | | PRECURSOR (LRP) | |
| | | sequence. | | | (ALPHA-2- | |
| | | | | | MACROGLOBULIN | |
| | | | | | RECEPTOR) | |
| | | | | | (A2MR)>PIR2:A53102 | |
| | | | | | LDL receptor-related | |
| | | | | | protein / alpha-2- | |
| | | | | | macroglobulin receptor | |
| | | | | | precursor - | |
| | | | | | chicken>GP:GGLRPA2 | |
| | | | | | MR_1 G; gallus mRNA | |
| | | | | | for LRP/alp | |
| 49 | U18795 | Saccharomyces | 1 | NKC1_SQUAC | BUMETANIDE− | 0.73 |
| | | cerevisiae | | | SENSITIVE SODIUM- | |
| | | chromosome V | | | (POTASSIUM)- | |
| | | cosmids 9669, | | | CHLORIDE | |
| | | 8334, 8199, and | | | COTRANSPORTER 2 | |
| | | lambda clone | | | (NA-K-CL | |
| | | 1160. | | | SYMPORTER)>PIR2:A | |
| | | | | | 53491 bumetanide- | |
| | | | | | sensitive Na-K-C1 | |
| | | | | | cotransporter - spiny | |
| | | | | | dogfish>GP:SANKCC1_{—} | |
| | | | | | 1 Squalus acanthias | |
| | | | | | bumetanide-sensitive Na- | |
| | | | | | K-C1 cotransport protein | |
| | | | | | (NKCC | |
| 50 | AC002523 | Homo sapiens ; | 1 | BXEN_CLOBO | BOTULINUM | 0.71 |
| | | HTGS phase 1, | | | NEUROTOXIN TYPE | |
| | | 54 unordered | | | E, NONTOXIC | |
| | | pieces. | | | COMPONENT>GP:CLO | |
| | | | | | ENT120_1 C; botulinum | |
| | | | | | gene for nontoxic | |
| | | | | | component of progenitor | |
| | | | | | toxin, complete cds | |
| 51 | AC002345 | *** | 1 | P3K2_DICDI | PHOSPHATIDYLINOSI | 0.58 |
| | | SEQUENCING | | | TOL 3-KINASE 2 (EC | |
| | | IN PROGRESS | | | 2.7.1.137) (PI3- | |
| | | *** Genomic | | | KINASE) (PTDINS-3- | |
| | | sequence from | | | KINASE) | |
| | | Human 17; | | | (PI3K)>GP:DDU23477_{—} | |
| | | HTGS phase 1, | | | 1 Dictyostelium | |
| | | 10 unordered | | | discoideum | |
| | | pieces. | | | phosphatidylinositol-4,5- | |
| | | | | | diphosphate 3-kinase | |
| | | | | | (PIK2) mRNA, complete | |
| | | | | | cds | |
| 52 | X14253 | Human mRNA | 1 | I55651 | noradrenaline transporter - | 0.55 |
| | | for cripto protein. | | | | |
| | | | | | bovine>GP:BTU09198_1 | |
| | | | | | Bos taurus noradrenaline | |
| | | | | | transporter mRNA, | |
| | | | | | complete cds | |
| 53 | U23516 | Caenorhabditis | 1 | I69024 | MHC sex-limited protein | 0.47 |
| | | elegans cosmid | | | - mouse | |
| | | B0416. | | | (fragment)>GP:MUSMH | |
| | | | | | C4AD_1 Mouse class III | |
| | | | | | H2-Slp sex-limited | |
| | | | | | protein gene, exons 1, 2 | |
| | | | | | and 3; MHC sex-limited | |
| | | | | | protein | |
| 54 | AB006698 | Arabidopsis | 1 | S81293_1 | L1 {insertion sequence, | 0.25 |
| | | thaliana genomic | | | provirus} [human | |
| | | DNA, | | | papillomavirus type 6b | |
| | | chromosome 5, | | | HPV6b, KP4, Genomic | |
| | | P1 clone: | | | Mutant, 121 nt]; Authors | |
| | | MCL 19. | | | note this reading frame | |
| | | | | | results from a 454 bp | |
| | | | | | deletion and resulting | |
| 55 | K03458 | Human | 1 | S13383 | hydroxyproline-rich | 0.24 |
| | | immunodeficienc | | | glycoprotein - sorghum | |
| | | y virus type 1, | | | | |
| | | isolate Zaire 6, | | | | |
| | | vif, tat, rev, env, | | | | |
| | | nef genes and 3′ | | | | |
| | | LTR. | | | | |
| 56 | B26794 | T1O16TR TAMU | 1 | RK34_PORPU | CHLOROPLAST 50S | 0.021 |
| | | Arabidopsis | | | RIBOSOMAL | |
| | | thaliana genomic | | | PROTEIN | |
| | | clone T1O16. | | | L34>PIR2:S73111 | |
| | | | | | ribosomal protein L34 - | |
| | | | | | red alga (Porphyra | |
| | | | | | purpurea) | |
| | | | | | chloroplast>GP:PPU388 | |
| | | | | | 04_4 Porphyra purpurea | |
| | | | | | chloroplast genome, | |
| | | | | | complete sequence; 50S | |
| | | | | | ribosomal protein L34 | |
| 57 | Z98950 | Human DNA | 1 | D41132 | collagen-related protein 4 | 0.02 |
| | | sequence *** | | | - Hydra magnipapillata | |
| | | SEQUENCING | | | (fragment)>PIR2:S21932 | |
| | | IN PROGRESS | | | mini-collagen - Hydra | |
| | | *** from clone | | | sp.>GP:HSNCOL4_1 | |
| | | 507I15; HTGS | | | Hydra N-COL 4 mRNA | |
| | | phase 1. | | | for mini-collagen; No | |
| | | | | | start codon | |
| 58 | U57057 | Human WD | 1 | DMU15602_1 | Drosophila melanogaster | 0.019 |
| | | protein IR10 | | | (zeste-white 4) mRNA, | |
| | | mRNA, complete | | | complete cds; Similar to | |
| | | cds. | | | C; elegans B0464; 4 gene | |
| | | | | | product, Swiss-Prot | |
| | | | | | Accession Number | |
| | | | | | Q03562 | |
| 59 | U57057 | Human WD | 1 | CR2_MOUSE | COMPLEMENT | 0.0074 |
| | | protein IR10 | | | RECEPTOR TYPE 2 | |
| | | mRNA, complete | | | PRECURSOR (CR2) | |
| | | cds. | | | (COMPLEMENT C3D | |
| | | | | | RECEPTOR)>PIR2:A43 | |
| | | | | | 526 complement | |
| | | | | | C3d/Epstein-Barr virus | |
| | | | | | receptor 2 precursor - | |
| | | | | | mouse>GP:MUSCR2AA | |
| | | | | | _1 Murine complement | |
| | | | | | receptor type 2 (CR2) | |
| | | | | | mRNA, complete cds; | |
| | | | | | Complement receptor | |
| | | | | | type | |
| 60 | B65337 | CIT-HSP- | 1 | A38096 | perlecan precursor - | 0.0051 |
| | | 2021H21.TF | | | human>GP:HUMHSPG2 | |
| | | CIT-HSP Homo | | | B_1 Human heparan | |
| | | sapiens genomic | | | sulfate proteoglycan | |
| | | clone 2021H21. | | | (HSPG2) mRNA, | |
| | | | | | complete cds | |
| 61 | U84722 | Human vascular | 1 | HSTAFII13_1 | H; sapiens mRNA for | 0.0012 |
| | | endothelial | | | TAFII135; Subunit of | |
| | | cadherin mRNA, | | | RNA polymerase II | |
| | | complete cds. | | | transcription factor | |
| | | | | | TFIID | |
| 62 | L41493 | Avian rotavirus | 1 | Y328_MYCPN | HYPOTHETICAL | 0.00015 |
| | | (strain turkey 1) | | | PROTEIN MG328 | |
| | | genomic segment | | | HOMOLOG>PIR2:S736 | |
| | | 4 outer capsid | | | 93 MG328 homolog | |
| | | protein (VP8*) | | | P01_orf1033 - | |
| | | gene. | | | Mycoplasma pneumoniae | |
| | | | | | (ATCC 29342) | |
| | | | | | (SGC3)>GP:MPAE0000 | |
| | | | | | 35_2 Mycoplasma | |
| | | | | | pneumoniae from bases | |
| | | | | | 442306 to 452472 | |
| | | | | | (section 35 of 63) of the | |
| | | | | | complete genome; | |
| | | | | | MG328 homolog, | |
| 63 | D63139 | Aeromonas sp. | 1 | MTCY16B7_3 | Mycobacterium | 6.30E−05 |
| | | gene for | | | tuberculosis cosmid | |
| | | chitinase, | | | SCY16B7; Unknown; | |
| | | complete and | | | MTCY16B7; 03, | |
| | | partial cds. | | | initiation factor, len: 900, | |
| | | | | | similar at C-terminal half | |
| | | | | | to eg IF2_BACSU | |
| | | | | | P17889 initiation factor | |
| | | | | | if-2 (716 aa), fasta | |
| 64 | J04974 | Human alpha-2 | 1 | GDF6_BOVIN | GROWTH/DIFFERENT | 1.00E−05 |
| | | type XI collagen | | | IATION FACTOR GDF- | |
| | | mRNA | | | 6 PRECURSOR | |
| | | (COL11A2). | | | (CARTILAGE− | |
| | | | | | DERIVED | |
| | | | | | MORPHOGENETIC | |
| | | | | | PROTEIN 2) (CDMP-2) | |
| | | | | | (FRAGMENT)>PIR2:B5 | |
| | | | | | 5452 cartilage-derived | |
| | | | | | morphogenetic protein 2 | |
| | | | | | precursor - bovine | |
| | | | | | (fragment)>GP:BTU136 | |
| | | | | | 61_1 Bos taurus | |
| | | | | | cartilage-derived morp | |
| 65 | AC002394 | Homo sapiens | 1 | CELC14F11_6 | Caenorhabditis elegans | 4.60E−06 |
| | | Chromosome 16 | | | cosmid C14F11; Similar | |
| | | BAC clone | | | to aspartate | |
| | | C1T987-SKA- | | | aminotransferase; coded | |
| | | 211C6 ˜complete | | | for by C; elegans cDNA | |
| | | genomic | | | CEMSF95FB; coded for | |
| | | sequence, | | | by C; elegans cDNA | |
| | | complete | | | yk41e4; 3; coded for by | |
| | | sequence. | | | C; elegans | |
| 66 | AB002312 | Human mRNA | 1 | NAT1_YEAST | N-TERMINAL | 1.00E−09 |
| | | for KIAA0314 | | | ACETYLTRANSFERAS | |
| | | gene, partial cds. | | | E 1 (EC 2.3.1.88) | |
| | | | | | (AMINO-TERMINAL, | |
| | | | | | ALPHA- AMINO, | |
| | | | | | ACETYLTRANSFERAS | |
| | | | | | E 1) | |
| 67 | AC003085 | Human BAC | 1 | DP19_CAEEL | DPY-19 | 4.20E−11 |
| | | clone RG094H21 | | | PROTEIN>PIR2:S44629 | |
| | | from 7q21-q22, | | | f22b7.10 protein - | |
| | | complete | | | Caenorhabditis | |
| | | sequence. | | | elegans >GP:CELF22B7_{—} | |
| | | | | | 9 C; aenorhabditis elegans | |
| | | | | | (Bristol N2) cosmid | |
| | | | | | F22B7; Putative | |
| 68 | X55026 | P. anserina | 1 | NAT1_YEAST | N-TERMINAL | 8.40E−12 |
| | | complete | | | ACETYLTRANSFERAS | |
| | | mitochondrial | | | E 1 (EC 2.3.1.88) | |
| | | genome. | | | (AMINO-TERMINAL, | |
| | | | | | ALPHA- AMINO, | |
| | | | | | ACETYLTRANSFERAS | |
| | | | | | E 1) | |
| 69 | Z95399 | Caenorhabditis | 1 | CER06B9_5 | Caenorhabditis elegans | 1.50E−24 |
| | | elegans DNA *** | | | cosmid R06B9, complete | |
| | | SEQUENCING | | | sequence; R06B9; b; | |
| | | IN PROGRESS | | | Protein predicted using | |
| | | *** from clone | | | Genefinder; preliminary | |
| | | Y39B6; HTGS | | | prediction | |
| | | phase 1. | | | | |
| 70 | AC002339 | Arabidopsis | 0.99 | POLG_BVDVS | GENOME | 1 |
| | | thaliana | | | POLYPROTEIN>PIR1: | |
| | | chromosome II | | | A44217 genome | |
| | | BAC T11A07 | | | polyprotein - bovine viral | |
| | | genomic | | | diarrhea virus (strain SD- | |
| | | sequence, | | | 1)>GP:BVDPOLYPRO_{—} | |
| | | complete | | | 1 Bovine viral diarrhea | |
| | | sequence. | | | virus polyprotein RNA, | |
| | | | | | complete cds; Putative | |
| 71 | Y08559 | B. subtilis urease | 0.99 | LRP_CAEEL | LOW-DENSITY | 1 |
| | | operon and | | | LIPOPROTEIN | |
| | | downstream | | | RECEPTOR-RELATED | |
| | | DNA. | | | PROTEIN PRECURSOR | |
| | | | | | (LRP)>PIR2:A47437 | |
| | | | | | LDL-receptor-related | |
| | | | | | protein - Caenorhabditis | |
| | | | | | elegans>GP:CEF29D11_{—} | |
| | | | | | 2 Caenorhabditis elegans | |
| | | | | | cosmid F29D11, | |
| | | | | | complete sequence; | |
| | | | | | F29D11; 1; Protein | |
| | | | | | predicted using Genefi | |
| 72 | U67548 | Methanococcus | 0.99 | YB60_YEAST | HYPOTHETICAL 16.3 | 1 |
| | | jannaschii from | | | KD PROTEIN IN | |
| | | bases 986219 to | | | DUR1, 2-NGR1 | |
| | | 996377 (section | | | INTERGENIC | |
| | | 90 of 150) of the | | | REGION>PIR2:S46084 | |
| | | complete | | | probable membrane | |
| | | genome. | | | protein YBR210w - yeast | |
| | | | | | (Saccharomyces | |
| | | | | | cerevisiae)>GP:SCYBR2 | |
| | | | | | 10W_1 S; cerevisiae | |
| | | | | | chromosome II reading | |
| | | | | | frame ORF YBR210w | |
| 73 | U51645 | Plasmodium | 0.99 | HPSVRPL_1 | Sin Nombre virus (NM | 0.99 |
| | | falciparum | | | H10) RNA L segment | |
| | | cytidine | | | encoding RNA | |
| | | triphosphate | | | polymerase (L protein), | |
| | | synthetase gene, | | | complete cds; Viral RNA | |
| | | complete cds. | | | polymerase (L protein); | |
| | | | | | Putative>GP:HPSVRPL | |
| | | | | | A_1 Sin Nombre virus | |
| | | | | | (NMR11) RNA L | |
| | | | | | segment encoding RNA | |
| | | | | | polymerase (L protein), | |
| | | | | | complete cds; Vir | |
| 74 | Z49889 | Caenorhabditis | 0.99 | MUSHDPRO | Mouse alternatively | 0.021 |
| | | elegans cosmid | | B_1 | spliced HD protein | |
| | | T06H11, | | | mRNA, complete cds | |
| | | complete | | | | |
| | | sequence. | | | | |
| 75 | Z69374 | Human DNA | 0.99 | NCPR_YEAST | NADPH- | 0.017 |
| | | sequence from | | | CYTOCHROME P450 | |
| | | cosmid L174G8, | | | REDUCTASE (EC | |
| | | Huntington's | | | 1.6.2.4) (CPR) | |
| | | Disease Region, | | | | |
| | | chromosome | | | | |
| | | 4p16.3 contains a | | | | |
| | | pair of ESTs. | | | | |
| 76 | Z35847 | S. cerevisiae | 0.99 | CYPA_CAEEL | PEPTIDYL-PROLYL | 0.0044 |
| | | chromosome II | | | CIS-TRANS | |
| | | reading frame | | | ISOMERASE 10 (EC | |
| | | ORF YBL086c. | | | 5.2.1.8) (PPIASE) | |
| | | | | | (ROTAMASE) | |
| | | | | | (CYCLOPHILIN- | |
| | | | | | 10)>GP:CELB0252_4 | |
| | | | | | Caenorhabditis elegans | |
| | | | | | cosmid B0252; Similar to | |
| | | | | | peptidyl-prolyl cis-trans | |
| | | | | | isomerase (PPIASE) | |
| | | | | | (CYCLOPHILIN)>GP:C | |
| | | | | | EU34954_1 | |
| | | | | | Caenorhabditis el | |
| 77 | L35330 | Rattus norvegicus | 0.99 | CELR148_1 | Caenorhabditis elegans | 0.0032 |
| | | glutathione S- | | | cosmid R148; Contains | |
| | | transferase Yb3 | | | similarity to drosophila | |
| | | subunit gene, | | | DNA-binding protein | |
| | | complete cds. | | | K10 (NID:g8148); coded | |
| | | | | | for by C; elegans cDNA | |
| | | | | | yk118e11; 5; coded for by | |
| | | | | | C; elegans cDNA | |
| 78 | Y00324 | Chicken | 0.99 | A56922 | transcription factor shn - | 0.0023 |
| | | vitellogenin gene | | | fruit fly (Drosophila | |
| | | 3′ flanking | | | melanogaster) | |
| | | region. | | | | |
| 79 | M32659 | D. melanogaster | 0.99 | OMU25146_1 | Oncorhynchus mykiss | 0.0017 |
| | | Shab11 protein | | | recombination activating | |
| | | mRNA, complete | | | protein 2 gene, partial | |
| | | cds. | | | cds | |
| 80 | Z69880 | H. sapiens | 0.99 | M84D_DRO | MALE SPECIFIC | 0.0011 |
| | | SERCA3 gene | | ME | SPERM PROTEIN | |
| | | (partial). | | | MST84DD>PIR2:S2577 | |
| | | | | | 5 testis-specific protein | |
| | | | | | Mst84Dd - fruit fly | |
| | | | | | (Drosophila | |
| | | | | | melanogaster)>GP:DMM | |
| | | | | | ST84D_4 | |
| | | | | | D; melanogaster | |
| | | | | | Mst84Da, Mst84Db, | |
| | | | | | Mst84Dc and Mst84Dd | |
| | | | | | genes for put; sperm | |
| | | | | | protein | |
| 81 | M99166 | Escherichia coli | 0.99 | MTU88962_1 | Mycobacterium | 6.50E−07 |
| | | Trp repressor | | | tuberculosis unknown | |
| | | binding protein | | | protein gene, partial cds | |
| | | (wrbA) gene, | | | | |
| | | complete cds. | | | | |
| 82 | X99257 | R. norvegicus | 0.99 | MIU68729_1 | Meloidogyne incognita | 1.60E−09 |
| | | mRNA for lamin | | | cuticle preprocollagen | |
| | | C2. | | | (col-2) mRNA, complete | |
| | | | | | cds; Putative | |
| 83 | AC002432 | Human BAC | 0.98 | 1FMDC | Foot and mouth disease | 0.14 |
| | | clone RG317G18 | | | virus type c-s8c1, chain | |
| | | from 7q31, | | | C - foot and mouth | |
| | | complete | | | disease virus type c-s8c1 | |
| | | sequence. | | | expressed in hamster | |
| | | | | | kidney cells | |
| 84 | Z34799 | Caenorhabditis | 0.98 | MMU57368_1 | Mus musculus EGF | 0.0028 |
| | | elegans cosmid | | | repeat transmembrane | |
| | | F34D10, | | | protein mRNA, complete | |
| | | complete | | | cds; Notch like repeats; | |
| | | sequence. | | | notch 2 | |
| 85 | B15207 | 344E15.TV | 0.98 | POLG_HCVJ6 | GENOME | 0.00083 |
| | | CIT978SKA1 | | | POLYPROTEIN | |
| | | Homo sapiens | | | (CONTAINS: CAPSID | |
| | | genomic clone A- | | | PROTEIN C (CORE | |
| | | 344E15. | | | PROTEIN); MATRIX | |
| | | | | | PROTEIN (ENVELOPE | |
| | | | | | PROTEIN M); MAJOR | |
| | | | | | ENVELOPE PROTEIN | |
| | | | | | E; NONSTRUCTURAL | |
| | | | | | PROTEINS NS1, NS2, | |
| | | | | | NS4A AND NS4B; | |
| | | | | | HELICASE (NS3); | |
| | | | | | RNA-DIRECTED RNA | |
| | | | | | POLYMERASE (EC | |
| | | | | | 2.7.7.48) (NS5))>PI | |
| 86 | AC002412 | *** | 0.98 | KDG1_ARATH | DIACYLGLYCEROL | 0.00024 |
| | | SEQUENCING | | | KINASE 1 (EC | |
| | | IN PROGRESS | | | 2.7.1.107) | |
| | | *** Human | | | (DIGLYCERIDE | |
| | | Chromosome X; | | | KINASE) (DGK 1) | |
| | | HTGS phase 1, 2 | | | (DAG KINASE | |
| | | unordered pieces. | | | 1)>PIR2:S71467 | |
| | | | | | diacylglycerol kinase | |
| | | | | | (EC 2.7.1.107) ATDGK1 | |
| | | | | | - Arabidopsis | |
| | | | | | thaliana>GP:ATHATDG | |
| | | | | | K1_1 Arabidopsis | |
| | | | | | thaliana mRNA for | |
| | | | | | diacylglycerol kinase, | |
| | | | | | complete c | |
| 87 | X57010 | Human COL2A1 | 0.98 | D80005_1 | Human mRNA for | 5.90E−10 |
| | | gene for collagen | | | KIAA0183 gene, partial | |
| | | II alpha 1 chain, | | | cds | |
| | | exons E2-E15. | | | | |
| 88 | M83093 | Neurospora | 0.98 | YA53_SCHPO | HYPOTHETICAL 24.2 | 3.00E−22 |
| | | crassa cAMP- | | | KD PROTEIN | |
| | | dependent protein | | | C13A11.03 IN | |
| | | kinase (cot-1) | | | CHROMOSOME | |
| | | gene, complete | | | I>GP:SPAC13A11_3 | |
| | | cds. | | | S; pombe chromosome I | |
| | | | | | cosmid c13A11; | |
| | | | | | Unknown; | |
| | | | | | SPAC13A11; 03 | |
| | | | | | unknown, len: 210 | |
| 89 | U96271 | Helicobacter | 0.97 | SLMEN6_1 | S; latifolia mRNA for | 0.43 |
| | | pylori heat shock | | | Men-6 | |
| | | protein 70 | | | protein>GP:SLMEN6_1 | |
| | | (hsp70) gene, | | | S; latifolia mRNA for | |
| | | complete cds. | | | Men-6 protein | |
| 90 | U49944 | Caenorhabditis | 0.97 | RON_HUMAN | MACROPHAGE | 0.034 |
| | | elegans cosmid | | | STIMULATING | |
| | | C39E6. | | | PROTEIN RECEPTOR | |
| | | | | | PRECURSOR (EC | |
| | | | | | 2.7.1.112)>PIR2:I38185 | |
| | | | | | protein-tyrosine kinase | |
| | | | | | (EC 2.7.1.112), receptor | |
| | | | | | type ron - | |
| | | | | | human>GP:HSRON_1 | |
| | | | | | H; sapiens RON mRNA | |
| | | | | | for tyrosine kinase; | |
| | | | | | Putative | |
| 91 | Y09255 | B. cereus dnaI | 0.97 | CELT05C1_5 | Caenorhabditis elegans | 0.00043 |
| | | gene, partial. | | | cosmid T05C1; Coded | |
| | | | | | for by C; elegans cDNA | |
| | | | | | yk30f6; 3; coded for by | |
| | | | | | C; elegans cDNA | |
| | | | | | yk34f10; 3 | |
| 92 | AC002413 | *** | 0.96 | CELC44E4_5 | Caenorhabditis elegans | 1 |
| | | SEQUENCING | | | cosmid C44E4; Weak | |
| | | IN PROGRESS | | | similarity to the | |
| | | *** Human | | | drosophila hyperplastic | |
| | | Chromosome X; | | | disc protein | |
| | | HTGS phase 1, 2 | | | (GB:L14644); coded for | |
| | | unordered pieces. | | | by C; elegans cDNA | |
| | | | | | yk49h6; 5; coded for by | |
| | | | | | C; elegans cDNA | |
| 93 | U41625 | Caenorhabditis | 0.96 | HMGC_HUM | HIGH MOBILITY | 1 |
| | | elegans cosmid | | AN | GROUP PROTEIN | |
| | | K03A1. | | | HMGI-C>PIR2:JC2232 | |
| | | | | | high mobility group I-C | |
| | | | | | phosphoprotein - | |
| | | | | | human>GP:HSHMGICG | |
| | | | | | 5_1 Human high- | |
| | | | | | mobility group | |
| | | | | | phosphoprotein isoform | |
| | | | | | I-C (HMGIC) gene, exon | |
| | | | | | 5>GP:HSHMGICP_1 | |
| | | | | | H; sapiens mRNA for | |
| | | | | | HMGI-C | |
| | | | | | protein>GP:HSHMGIC | |
| 94 | Z82202 | Human DNA | 0.96 | YTH3_CAEEL | HYPOTHETICAL 75.5 | 0.73 |
| | | sequence *** | | | KD PROTEIN C14A4.3 | |
| | | SEQUENCING | | | IN CHROMOSOME | |
| | | IN PROGRESS | | | II>GP:CEC14A4_3 | |
| | | *** from clone | | | Caenorhabditis elegans | |
| | | 34P24; HTGS | | | cosmid C14A4, complete | |
| | | phase 1. | | | sequence; C14A4; 3; | |
| | | | | | Weak similarity with a B; | |
| | | | | | Flavum translocation | |
| | | | | | protein (Swiss Prot | |
| | | | | | accession number | |
| | | | | | P38376) | |
| 95 | AL008734 | Human DNA | 0.96 | S25299 | extensin precursor (clone | 0.0004 |
| | | sequence *** | | | Tom L-4) - | |
| | | SEQUENCING | | | tomato>GP:TOMEXTE | |
| | | IN PROGRESS | | | NB_1 L; esculentum | |
| | | *** from clone | | | extensin (class II) gene, | |
| | | 324M8; HTGS | | | complete cds | |
| | | phase 1. | | | | |
| 96 | L15388 | Human G | 0.96 | HUMCOL7A1 | Homo sapiens (clones: | 4.60E−06 |
| | | protein-coupled | | X_1 | CW52-2, CW27-6, | |
| | | receptor kinase | | | CW15-2, CW26-5, 11- | |
| | | (GRK5) mRNA, | | | 67) collagen type VII | |
| | | complete cds. | | | intergenic region and | |
| | | | | | (COL7A1) gene, | |
| | | | | | complete cds | |
| 97 | X97384 | A. thaliana atran3 | 0.95 | <NONE> | <NONE> | <NONE> |
| | | gene. | | | | |
| 98 | M62505 | Human C5a | 0.95 | RIPB₋ BRYDI | RIBOSOME− | 0.83 |
| | | anaphylatoxin | | | INACTIVATING | |
| | | receptor mRNA, | | | PROTEIN BRYODIN | |
| | | complete cds. | | | (RRNA N- | |
| | | | | | GLYCOSIDASE) (EC | |
| | | | | | 3.2.2.22) | |
| | | | | | (FRAGMENT)>PIR2:S1 | |
| | | | | | 6491 rRNA N- | |
| | | | | | glycosidase (EC | |
| | | | | | 3.2.2.22) bryodin - red | |
| | | | | | bryony (fragment) | |
| 99 | D28778 | Cucumber mosaic | 0.95 | POLS_RUBVM | STRUCTURAL | 0.00037 |
| | | virus RNA 1 for | | | POLYPROTEIN | |
| | | 1a, complete | | | (CONTAINS: | |
| | | sequence. | | | NUCLEOCAPSID | |
| | | | | | PROTEIN C; | |
| | | | | | MEMBRANE | |
| | | | | | GLYCOPROTEINS E1 | |
| | | | | | AND | |
| | | | | | E2)>PIR1:GNWVR3 | |
| | | | | | structural polyprotein - | |
| | | | | | rubella virus (strain | |
| | | | | | M33)>GP:TORUB24S_1 | |
| | | | | | Rubella virus 24S | |
| | | | | | subgenomic mRNA for | |
| | | | | | structural proteins E1, E2 | |
| | | | | | and C; | |
| 100 | AF016202 | Homo sapiens | 0.93 | HSU79716_1 | Human reelin (RELN) | 1 |
| | | immunoglobulin | | | mRNA, complete cds | |
| | | heavy chain | | | | |
| | | CDR3 gene, | | | | |
| | | partial cds. | | | | |
| 101 | Z68303 | Caenorhabditis | 0.93 | HS5HT4SAR_1 | H; sapiens mRNA for | 0.87 |
| | | elegans cosmid | | | serotonin 4SA receptor | |
| | | ZK809, complete | | | (5-HT4SA-R) | |
| | | sequence. | | | | |
| 102 | X03049 | E. coli DNA | 0.93 | S37594 | mucin - human | 0.0019 |
| | | sequence 5′ to | | | (fragment) | |
| | | origin of | | | | |
| | | replication oriC. | | | | |
| 103 | M32659 | D. melanogaster | 0.93 | S38480 | nonstructural protein - | 2.30E−06 |
| | | Shab11 protein | | | rubella | |
| | | mRNA, complete | | | virus>GP:RVM33NP_1 | |
| | | cds. | | | Rubella virus M33 RNA | |
| | | | | | for a nonstructural | |
| | | | | | protein; Nonstructural | |
| | | | | | protein genes | |
| 104 | D88687 | Human mRNA | 0.93 | BAT3_HUMAN | LARGE PROLINE− | 8.70E−07 |
| | | for KM-102- | | | RICH PROTEIN BAT3 | |
| | | derived | | | (HLA-B-ASSOCIATED | |
| | | reductase-like | | | TRANSCRIPT | |
| | | factor, complete | | | 3)>PIR2:A35098 MHC | |
| | | cds. | | | class III | |
| | | | | | histocompatibility | |
| | | | | | antigen HLA-B- | |
| | | | | | associated transcript 3 - | |
| | | | | | human>GP:HUMBAT3 | |
| | | | | | A_1 Human HLA-B- | |
| | | | | | associated transcript 3 | |
| | | | | | (BAT3) mRNA, | |
| | | | | | complete | |
| | | | | | cds>GP:HUMBAT3 | |
| 105 | D16847 | Mouse mRNA for | 0.93 | S52796 | prpL2 protein - human | 3.20E−08 |
| | | stromal cell | | | (fragment)>GP:HSPRPL | |
| | | derived protein-1, | | | 2_1 H; sapiens mRNA for | |
| | | complete cds. | | | PRPL-2 protein | |
| 106 | D90915 | Synechocystis sp. | 0.92 | YEK9_YEAST | HYPOTHETICAL 53.9 | 5.90E−05 |
| | | PCC6803 | | | KD PROTEIN IN AFG3- | |
| | | complete | | | SEB2 INTERGENIC | |
| | | genome, 17/27, | | | REGION>PIR2:S50477 | |
| | | 2137259- | | | hypothetical protein | |
| | | 2267259. | | | YER019w - yeast | |
| | | | | | (Saccharomyces | |
| | | | | | cerevisiae)>GP:SCE9537 | |
| | | | | | _20 Saccharomyces | |
| | | | | | cerevisiae chromosome | |
| | | | | | V cosmids 9537, 9581, | |
| | | | | | 9495, 9867, and lambda | |
| | | | | | clone 5898 | |
| 107 | AJ001101 | Mus musculus | 0.92 | DMU58282_1 | Drosophila melanogaster | 3.50E−05 |
| | | mRNA for | | | Bowel (bowl) mRNA, | |
| | | gC1qBP gene. | | | complete cds; | |
| | | | | | Transcription factor; | |
| | | | | | C2H2 zinc finger protein; | |
| | | | | | zinc fingers have | |
| | | | | | extensive sequence | |
| | | | | | similarity to Drosophila | |
| | | | | | odd-skipped | |
| 108 | X57108 | Human gene for | 0.92 | S69032 | hypothetical protein | 4.30E−21 |
| | | cerebroside | | | YPR144c - yeast | |
| | | sulfate activator | | | (Saccharomyces | |
| | | protein, exons 10- | | | cerevisiae)>GP:YSCP96 | |
| | | 14. | | | 59_17 Saccharomyces | |
| | | | | | cerevisiae chromosome | |
| | | | | | XVI cosmid 9659; | |
| | | | | | Ypr144cp; Weak | |
| | | | | | similarity near C- | |
| | | | | | terminus to RNA | |
| | | | | | Polymerase beta subunit | |
| | | | | | (Swiss Prot; accession | |
| | | | | | number P11213) | |
| 109 | D14635 | Caenorhabditis | 0.91 | YM13_YEAST | PUTATIVE ATP- | 0.69 |
| | | elegans DNA for | | | DEPENDENT RNA | |
| | | EMB-5. | | | HELICASE | |
| | | | | | YMR128W>PIR2:S5305 | |
| | | | | | 8 probable membrane | |
| | | | | | protein YMR128w - | |
| | | | | | yeast (Saccharomyces | |
| | | | | | cerevisiae)>GP:SC9553_{—} | |
| | | | | | 4 S; cerevisiae | |
| | | | | | chromosome XIII cosmid | |
| | | | | | 9553; Unknown; | |
| | | | | | YM9553; 04, probable | |
| | | | | | ATP-dependent RNA | |
| | | | | | helicase, len: | |
| 110 | B55500 | CIT-HSP- | 0.91 | U97553_79 | Murine herpesvirus 68 | 0.00016 |
| | | 387J2.TFB CIT- | | | strain WUMS, complete | |
| | | HSP Homo | | | genome; Unknown | |
| | | sapiens genomic | | | | |
| | | clone 387J2. | | | | |
| 111 | X03049 | E. coli DNA | 0.9 | POL_MLVAV | POL POLYPROTEIN | 0.0019 |
| | | sequene 5′ to | | | (PROTEASE (EC | |
| | | origin of | | | 3.4.23.-); REVERSE | |
| | | replication oriC. | | | TRANSCRIPTASE (EC | |
| | | | | | 2.7.7.49); | |
| | | | | | RIBONUCLEASE H | |
| | | | | | (EC | |
| | | | | | 3.1.26.4))>PIR1:GNMV | |
| | | | | | GV pol polyprotein - | |
| | | | | | AKV murine leukemia | |
| | | | | | virus | |
| 112 | U91327 | Human | 0.89 | JC5568 | serine protease (EC 3.4.- | 1 |
| | | chromosome | | | .-) h1 - Serratia | |
| | | 12p15 BAC clone | | | marcescens | |
| | | CIT987SK-99D8 | | | | |
| | | complete | | | | |
| | | sequence. | | | | |
| 113 | X13295 | Rat mRNA for | 0.89 | MNGPOLY_1 | Mengo virus polyprotein | 1 |
| | | alpha-2u | | | genome, complete cds | |
| | | globulin-related | | | withe repeats | |
| | | protein. | | | | |
| 114 | Z78415 | Caenorhabditis | 0.89 | AB000121_1 | Mouse mRNA for | 0.39 |
| | | elegans cosmid | | | TBPIP, complete cds; | |
| | | C17G1, complete | | | TBP1 interacting protein | |
| | | sequence. | | | | |
| 115 | AC002308 | *** | 0.88 | YLK2_CAEEL | HYPOTHETICAL 122.7 | 0.0037 |
| | | SEQUENCING | | | KD PROTEIN D1044.2 | |
| | | IN PROGRESS | | | IN CHROMOSOME | |
| | | *** Human | | | III>GP:CELD1044_4 | |
| | | Chromosome | | | Caenorhabditis elegans | |
| | | 22q11 BAC | | | cosmid D1044 | |
| | | Clone 1000e4; | | | | |
| | | HTGS phase 1, | | | | |
| | | 26 unordered | | | | |
| | | pieces. | | | | |
| 116 | AC002073 | Human PAC | 0.88 | S28499 | probable finger protein - | 1.10E−31 |
| | | clone DJ515N1 | | | rat>GP:RNZFP_1 | |
| | | from 22q11.2- | | | R; norvegicus mRNA for | |
| | | q22, complete | | | putative zinc finger | |
| | | sequence. | | | protein | |
| 117 | Z83848 | Human DNA | 0.87 | NDL_DROME | SERINE PROTEASE | 1 |
| | | sequence *** | | | NUDEL PRECURSOR | |
| | | SEQUENCING | | | (EC 3.4.21.- | |
| | | IN PROGRESS | | | )>PIR2:A57096 nudel | |
| | | *** from clone | | | protein precursor - fruit | |
| | | 57A13; HTGS | | | fly (Drosophila | |
| | | phase 1. | | | melanogaster)>GP:DMU | |
| | | | | | 29153_1 Drosophila | |
| | | | | | melanogaster nudel (ndl) | |
| | | | | | mRNA, complete cds; | |
| | | | | | Serine protease; Soma | |
| | | | | | dependent gene required | |
| | | | | | matern | |
| 118 | U23449 | Caenorhabditis | 0.87 | AF023268_3 | Homo sapiens clk2 | 0.21 |
| | | elegans cosmid | | | kinase (CLK2), propin1, | |
| | | K06A1. | | | cote1, glucocerebrosidase | |
| | | | | | (GBA), and metaxin | |
| | | | | | genes, complete cds; | |
| | | | | | metaxin pseudogene and | |
| | | | | | glucocerebrosidase | |
| | | | | | pseudogene; and | |
| | | | | | thrombospondin3 | |
| | | | | | (THBS3) | |
| 119 | Z68181 | H. vulgaris | 0.87 | RABCY450C | Rabbit cytochrome P-450 | 0.14 |
| | | mRNA for | | _1 | gene, clone pP-450PBc3, | |
| | | elongation factor | | | 3′ end | |
| | | EF1-alpha. | | | | |
| 120 | AC000033 | Homo sapiens | 0.87 | VWF_CANFA | VON WILLEBRAND | 0.036 |
| | | chromosome 9, | | | FACTOR | |
| | | complete | | | PRECURSOR>GP:DOG | |
| | | sequence. | | | VWG_1 Canis familiaris | |
| | | | | | von Willebrand factor | |
| | | | | | mRNA, complete cds | |
| 121 | U23449 | Caenorhabditis | 0.86 | S48988_1 | CRP-1=cystatin-related | 0.64 |
| | | elegans cosmid | | | protein [rats, Wistar | |
| | | K06A1. | | | albino, mRNA Partial, | |
| | | | | | 213 nt]; Cystatin-related | |
| | | | | | protein; Method: | |
| | | | | | conceptual translation | |
| | | | | | supplied by author; This | |
| | | | | | sequence comes from | |
| | | | | | Fig; | |
| 122 | Z89651 | F. rubripes GSS | 0.86 | CPU65981_1 | Cryptosporidium parvum | 0.6 |
| | | sequence, clone | | | P-ATPase gene (CppA- | |
| | | 090I24cD5. | | | E1) gene, complete cds; | |
| | | | | | Putative calcium-ATPase | |
| 123 | Z94055 | Human DNA | 0.86 | GLTB_SYNY3 | FERREDOXIN- | 0.03 |
| | | sequence from | | | DEPENDENT | |
| | | PAC 24M15 on | | | GLUTAMATE | |
| | | chromosome 1. | | | SYNTHASE 1 (EC | |
| | | Contains | | | 1.4.7.1) (FD- | |
| | | tenascin-R | | | GOGAT)>PIR2:S60228 | |
| | | (restrictin), EST. | | | glutamate synthase | |
| | | | | | (ferredoxin) (EC 1.4.7.1) | |
| | | | | | gltB - Synechocystis sp. | |
| | | | | | (PCC | |
| | | | | | 6803)>GP:D90902_66 | |
| | | | | | Synechocystis sp; | |
| | | | | | PCC6803 complete | |
| | | | | | genome, 4/27, 402290- | |
| | | | | | 524345; Gluta | |
| 124 | Z49250 | Human DNA | 0.86 | TRSCAPSID_1 | Tobacco ringspot virus | 3.00E−06 |
| | | sequence from | | | capsid protein gene, | |
| | | cosmid HW2, | | | complete cds | |
| | | Huntington's | | | | |
| | | Disease Region, | | | | |
| | | chromosome | | | | |
| | | 4p16.3. | | | | |
| 125 | Z92855 | Caenorhabditis | 0.84 | AE000809_8 | Methanobacterium | 1 |
| | | elegans DNA *** | | | thermoautotrophicum | |
| | | SEQUENCING | | | from bases 161632 to | |
| | | IN PROGRESS | | | 172569 (section 15 of | |
| | | *** from clone | | | 148) of the complete | |
| | | Y48C3; HTGS | | | genome; Aspartyl- tRNA | |
| | | phase 1. | | | synthetase; Function | |
| | | | | | Code:10; 07 - Metabolism | |
| | | | | | of | |
| 126 | AC002340 | *** | 0.83 | CET01E8_3 | Caenorhabditis elegans | 0.86 |
| | | SEQUENCING | | | cosmid T01E8, complete | |
| | | IN PROGRESS | | | sequence; T01E8; 3; | |
| | | *** Arabidopsis | | | Similar to 1- | |
| | | thaliana ‘TAMU’ | | | phosphatidylinositol-4,5- | |
| | | BAC ‘T11J7’ | | | bisphosphate | |
| | | genomic | | | phosphodiesterase; | |
| | | sequence near | | | cDNA EST CEESG02F | |
| | | marker ‘m283’; | | | comes from this gene; | |
| | | HTGS phase 1, 2 | | | | |
| | | unordered pieces. | | | | |
| 127 | AL008716 | Human DNA | 0.83 | HIVU51189_5 | HIV-1 clone 93th253 | 0.86 |
| | | sequence *** | | | from Thailand, complete | |
| | | SEQUENCING | | | genome; Tat protein | |
| | | IN PROGRESS | | | | |
| | | *** from clone | | | | |
| | | 206C7; HTGS | | | | |
| | | phase 1. | | | | |
| 128 | AC002340 | *** | 0.83 | S60257 | meltrin alpha - | 0.0013 |
| | | SEQUENCING | | | mouse>GP:MUSMAB_1 | |
| | | IN PROGRESS | | | Mouse mRNA for | |
| | | *** Arabidopsis | | | meltrin alpha, complete | |
| | | thaliana ‘TAMU’ | | | cds | |
| | | BAC ‘T11J7’ | | | | |
| | | genomic | | | | |
| | | sequence near | | | | |
| | | marker ‘m283’; | | | | |
| | | HTGS phase 1, 2 | | | | |
| | | unordered pieces. | | | | |
| 129 | Z83848 | Human DNA | 0.82 | ARO1_PNECA | PENTAFUNCTIONAL | 0.0098 |
| | | sequence *** | | | AROM POLYPEPTIDE | |
| | | SEQUENCING | | | (CONTAINS: 3- | |
| | | IN PROGRESS | | | DEHYDROQUINATE | |
| | | *** from clone | | | SYNTHASE (EC | |
| | | 57A13; HTGS | | | 4.6.1.3), 3- | |
| | | phase 1. | | | DEHYDROQUINATE | |
| | | | | | DEHYDRATASE (EC | |
| | | | | | 4.2.1.10) (3- | |
| | | | | | DEHYDROQUINASE), | |
| | | | | | SHIKIMATE 5- | |
| | | | | | DEHYDROGENASE | |
| | | | | | (EC 1.1.1.25), | |
| | | | | | SHIKIMATE KINASE | |
| | | | | | (EC 2.7.1.71), AND | |
| | | | | | EPSP SYNTHASE (E | |
| 130 | AF029308 | Homo sapiens | 0.8 | CELZK84_5 | Caenorhabditis elegans | 2.00E−08 |
| | | chromosome 9 | | | cosmid ZK84; Final exon | |
| | | duplication of the | | | in repeat region; similar | |
| | | T cell receptor | | | to long tandem repeat | |
| | | beta locus and | | | region of sialidase | |
| | | trypsinogen gene | | | (SP:TCNA_TRYCR, | |
| | | families. | | | P23253) and | |
| | | | | | neurofilament H protein; | |
| | | | | | coded for by C; elegans | |
| 131 | AC002458 | Human BAC | 0.78 | IGF2_PIG | INSULIN-LIKE | 0.44 |
| | | clone RG098M04 | | | GROWTH FACTOR II | |
| | | from 7q21-q22, | | | PRECURSOR (IGF- | |
| | | complete | | | II)>GP:SSIGF2_1 | |
| | | sequence. | | | S; scrofa mRNA IGF2 for | |
| | | | | | insulin-like-growth factor | |
| | | | | | 2; Insulin-like-growth | |
| | | | | | factor 2 preproprotein | |
| 132 | Z83843 | Human DNA | 0.78 | PAR51A_1 | P; tetraurelia 51A surface | 0.0014 |
| | | sequence *** | | | protein gene, complete | |
| | | SEQUENCING | | | cds | |
| | | IN PROGRESS | | | | |
| | | *** from clone | | | | |
| | | 368A4; HTGS | | | | |
| | | phase 1. | | | | |
| 133 | X03021 | Human gene for | 0.78 | CEF57B1_3 | Caenorhabditis elegans | 2.20E−05 |
| | | granulocyte- | | | cosmid F57B1, complete | |
| | | macrophage | | | sequence; F57B1; 3; | |
| | | colony | | | Protein predicted using | |
| | | stimulating factor | | | Genefinder; similar to | |
| | | (GM-CSF). | | | collagen | |
| 134 | Z74825 | S. cerevisiae | 0.77 | SYLM_SCHPO | PUTATIVE LEUCYL- | 0.96 |
| | | chromosome XV | | | TRNA SYNTHETASE, | |
| | | reading frame | | | MITOCHONDRIAL | |
| | | ORF YOL083w. | | | PRECURSOR (EC | |
| | | | | | 6.1.1.4) (LEUCINE− | |
| | | | | | TRNA | |
| | | | | | LIGASE)>PIR2:S62486 | |
| | | | | | hypothetical protein | |
| | | | | | SPAC4G8.09 - fission | |
| | | | | | yeast | |
| | | | | | (Schizosaccharomyces | |
| | | | | | pombe)>GP:SPAC4G8_{—} | |
| | | | | | 9 S; pombe chromosome I | |
| | | | | | cosmid c4G8; Unknown; | |
| | | | | | SPAC | |
| 135 | Z74825 | S. cerevisiae | 0.77 | RNU59809_1 | Rattus norvegicus | 0.01 |
| | | chromosome XV | | | mannose 6- | |
| | | reading frame | | | phosphate/insulin-like | |
| | | ORF YOL083w. | | | growth factor II receptor | |
| | | | | | (M6P/IGF2r) mRNA, | |
| | | | | | complete cds; Also | |
| | | | | | termed IGF-II/Man 6-P | |
| | | | | | receptor, MPR, CI-MPR | |
| 136 | U80445 | Caenorhabditis | 0.76 | S28499 | probable finger protein - | 1.10E−31 |
| | | elegans cosmid | | | rat>GP:RNZFP_1 | |
| | | C50F2. | | | R; norvegicus mRNA for | |
| | | | | | putative zinc finger | |
| | | | | | protein | |
| 137 | Z78545 | Caenorhabditis | 0.75 | RRU73586_1 | Rattus norvegicus | 0.023 |
| | | elegans cosmid | | | Fanconi anemia group C | |
| | | M03B6, complete | | | mRNA, complete cds; | |
| | | sequence. | | | Fanconi anemia group C | |
| | | | | | protein; Similar to human | |
| | | | | | FAC protein, GenBank | |
| | | | | | Accession Numbers | |
| | | | | | X66893 and X66894 | |
| 138 | Z97630 | Human DNA | 0.74 | HSMSHREC | H; sapiens mRNA for | 0.036 |
| | | sequence *** | | A_1 | MSH receptor; Author- | |
| | | SEQUENCING | | | given protein sequence is | |
| | | IN PROGRESS | | | in conflict with the | |
| | | *** from clone | | | conceptual translation | |
| | | 466N1; HTGS | | | | |
| | | phase 1. | | | | |
| 139 | AF007269 | Arabidopsis | 0.71 | HSU95090_1 | Homo sapiens | 0.16 |
| | | thaliana BAC | | | chromosome 19 cosmid | |
| | | IG002N01. | | | F19541, complete | |
| | | | | | sequence; F19541_1; | |
| | | | | | Hypothetical (partial) | |
| | | | | | protein similar to proline | |
| | | | | | oxidase | |
| 140 | AC002393 | Mouse | 0.7 | RNLTBP2_1 | Rattus norvegicus mRNA | 4.40E−05 |
| | | BAC284H12 | | | for LTBP-2 like protein; | |
| | | Chromosome 6, | | | Latent TGF- beta binding | |
| | | complete | | | protein-2 like protein | |
| | | sequence. | | | | |
| 141 | B15232 | 344G8.TV | 0.67 | DMSEVL2_2 | Drosophila melanogaster | 0.41 |
| | | CIT978SKA1 | | | sevenless mRNA; Put; | |
| | | Homo sapiens | | | sevenless protein (AA 1 - | |
| | | genomic clone A- | | | 2510) | |
| | | 344G08. | | | | |
| 142 | D13748 | Human mRNA | 0.66 | MMU53563_1 | Mus musculus Brg1 | 0.00016 |
| | | for eukaryotic | | | mRNA, partial cds; N- | |
| | | initiation factor | | | terminal region of the | |
| | | 4AI. | | | protein | |
| 143 | S45791 | band 3-related | 0.66 | POLS_RUBVR | STRUCTURAL | 5.60E−05 |
| | | protein=renal | | | POLYPROTEIN | |
| | | anion exchanger | | | (CONTAINS: | |
| | | AE2 homolog | | | NUCLEOCAPSID | |
| | | [rabbits, New | | | PROTEIN C; | |
| | | Zealand White, | | | MEMBRANE | |
| | | ileal epithelial | | | GLYCOPROTEINS E1 | |
| | | cells, mRNA, | | | AND | |
| | | 3964 nt]. | | | E2)>PIR1:GNWVRA | |
| | | | | | structural polyprotein - | |
| | | | | | rubella virus (strain | |
| | | | | | RA27/3 | |
| | | | | | vaccine)>GP:RUBCE21_{—} | |
| | | | | | 1 Rubella virus RA27/3 | |
| | | | | | RNA for capsid, E2 and | |
| | | | | | E1 proteins; Poly | |
| 144 | M22462 | Chicken protein | 0.66 | HSHP8PROT | H; sapiens mRNA for | 2.00E−06 |
| | | p54 (ets-1) | | _1 | HP8 protein; HP8 | |
| | | mRNA, complete | | | peptide | |
| | | cds. | | | | |
| 145 | U27999 | Human clone | 0.65 | CA18_HUMAN | COLLAGEN ALPHA | 5.70E−06 |
| | | pDEL52A11 | | | 1(VIII) CHAIN | |
| | | HLA-C region | | | PRECURSOR | |
| | | cosmid 52 | | | (ENDOTHELIAL | |
| | | genomic survey | | | COLLAGEN)>PIR2:S15 | |
| | | sequence. | | | 435 collagen alpha | |
| | | | | | 1(VIII) chain precursor - | |
| | | | | | human>GP:HSCOL8A1_{—} | |
| | | | | | 1 Human COL8A1 | |
| | | | | | mRNA for alpha 1(VIII) | |
| | | | | | collagen | |
| 146 | M54787 | N. crassa mating | 0.64 | I50717 | vacuolar H+-ATPase A | 0.0046 |
| | | type a-1 protein | | | subunit - chicken | |
| | | (mt a-1) gene, | | | (fragment)>GP:GGU220 | |
| | | exons 1- 3. | | | 78_1 Gallus gallus | |
| | | | | | vacuolar H+-ATPase A | |
| | | | | | subunit gene, partial cds | |
| 147 | AC002094 | Genomic | 0.63 | PVPVA1_1 | P; vivax pva1 gene | 0.1 |
| | | sequence from | | | | |
| | | Human 17, | | | | |
| | | complete | | | | |
| | | sequence. | | | | |
| 148 | U32701 | Haemophilus | 0.63 | FABG_HAEIN | 3-OXOACYL-[ACYL- | 2.00E−12 |
| | | influenzae from | | | CARRIER PROTEIN] | |
| | | bases 165345 to | | | REDUCTASE (EC | |
| | | 176101 (section | | | 1.1.1.100) (3- | |
| | | 16 of 163) of the | | | KETOACYL-ACYL | |
| | | complete | | | CARRIER PROTEIN | |
| | | genome. | | | REDUCTASE)>PIR2:D6 | |
| | | | | | 4051 3-oxoacyl-[acyl- | |
| | | | | | carrier-protein] reductase | |
| | | | | | (EC 1.1.1.100) - | |
| | | | | | Haemophilus influenzae | |
| | | | | | (strain Rd | |
| | | | | | KW20)>GP:HIU32701_{—} | |
| | | | | | 7 Haemophilus | |
| 149 | Z37159 | T. brucei serum | 0.61 | <NONE> | <NONE> | <NONE> |
| | | resistance | | | | |
| | | associated (SRA) | | | | |
| | | mRNA for VSG- | | | | |
| | | like protein. | | | | |
| 150 | AF027865 | Mus musculus | 0.61 | A56514 | chromokinesin - | 0.045 |
| | | Major | | | chicken>GP:GGU18309 | |
| | | Histocompatibilit | | | _1 Gallus gallus | |
| | | y Locus class II | | | chromokinesin mRNA, | |
| | | region. | | | complete cds | |
| 151 | U40938 | Caenorhabditis | 0.61 | YA53_SCHPO | HYPOTHETICAL 24.2 | 1.90E−24 |
| | | elegans cosmid | | | KD PROTEIN | |
| | | D1009. | | | C13A11.03 IN | |
| | | | | | CHROMOSOME | |
| | | | | | I>GP:SPAC13A11_3 | |
| | | | | | S; pombe chromosome I | |
| | | | | | cosmid c13A11; | |
| | | | | | Unknown; | |
| | | | | | SPAC13A11; 03, | |
| | | | | | unknown, len: 210 | |
| 152 | I16670 | Sequence 1 from | 0.59 | CELF21F8_7 | Caenorhabditis elegans | 0.39 |
| | | patent US | | | cosmids F21F8; Similar to | |
| | | 5476781. | | | eukaryotic aspartyl | |
| | | | | | proteases | |
| 153 | Z84468 | Human DNA | 0.59 | CLG1_YEAST | CYCLIN-LIKE | 0.0015 |
| | | sequence *** | | | PROTEIN | |
| | | SEQUENCING | | | CLG1>PIR2:S37607 | |
| | | IN PROGRESS | | | cyclin-like protein | |
| | | *** from clone | | | YGL215w - yeast | |
| | | 299D3; HTGS | | | (Saccharomyces | |
| | | phase 1. | | | cerevisiae)>GP:SCYGL2 | |
| | | | | | 15W_1 S; cerevisiae | |
| | | | | | chromosome VII reading | |
| | | | | | frame ORF | |
| | | | | | YGL215w>GP:YSCCLG | |
| | | | | | 1CPR_1 Saccharomyces | |
| | | | | | cerevisiae cyclin-like | |
| | | | | | protein (CLG1) gene | |
| 154 | U00054 | Caenorhabditis | 0.57 | <NONE> | <NONE> | <NONE> |
| | | elegans cosmid | | | | |
| | | K07E12. | | | | |
| 155 | M21207 | Synthetic SV40 T | 0.57 | 1CJL2 | cathepsin L (EC | 0.43 |
| | | antigen mutant | | | 3.4.22.15) mutant | |
| | | pseudogene, 3′ | | | (F(78P)L, C25S, T110A, | |
| | | end. | | | E176G, D178G), | |
| | | | | | fragment 2 - human | |
| 156 | AF020282 | Dictyostelium | 0.56 | AC002125_4 | Homo sapiens DNA from | 0.6 |
| | | discoideum | | | chromosome 19-cosmid | |
| | | DG2033 gene, | | | F25965, genomic | |
| | | partial cds. | | | sequence, complete | |
| | | | | | sequence; F25965_5; | |
| | | | | | Hypothetical 35; 3 kDa | |
| | | | | | protein similar to | |
| | | | | | GTPase-activating | |
| | | | | | proteins and orf3 from | |
| 157 | M86352 | Stigmatella | 0.56 | AC002398_4 | Human DNA from | 4.50E−06 |
| | | aurantiaca reverse | | | chromosome 19-specific | |
| | | transcriptase (163 | | | cosmid F25965, genomic | |
| | | RT) gene, | | | sequence, complete | |
| | | complete cds. | | | sequence; F25965_3; | |
| | | | | | Hypothetical 96 kDa | |
| | | | | | human protein similar to | |
| | | | | | alpha chimaerin; | |
| | | | | | Hypothetical | |
| | | | | | protein>GP:AC002398_4 | |
| | | | | | Human DNA from | |
| | | | | | chromosome 19-specific | |
| | | | | | cosmi | |
| 158 | AC003101 | *** | 0.54 | <NONE> | <NONE> | <NONE> |
| | | SEQUENCING | | | | |
| | | IN PROGRESS | | | | |
| | | *** Homo | | | | |
| | | sapiens | | | | |
| | | chromosome 17, | | | | |
| | | clone | | | | |
| | | HRPC41C23; | | | | |
| | | HTGS phase 1, | | | | |
| | | 33 unordered | | | | |
| | | pieces. | | | | |
| 159 | B12117 | F5L15-T7 IGF | 0.54 | CEF32H2_5 | Caenorhabditis elegans | 1 |
| | | Arabidopsis | | | cosmid F32H2, complete | |
| | | thaliana genomic | | | sequence; F32H2; 5; | |
| | | clone F5L15. | | | Similarity to Chicken | |
| | | | | | fatty acid synthase | |
| | | | | | (SW:P12276); cDNA | |
| | | | | | EST yk16c2; 5 comes | |
| | | | | | from this gene; cDNA | |
| | | | | | EST yk113h6; 5 comes | |
| 160 | AE000664 | Mus musculus | 0.54 | CET01G9_6 | Caenorhabditis elegans | 0.84 |
| | | TCR beta locus | | | cosmid T01G9, complete | |
| | | from bases | | | sequence; T01G9; 4; | |
| | | 250554 to 501917 | | | CDNA EST yk29b7; 5 | |
| | | (section 2 of 3) of | | | comes from this gene | |
| | | the complete | | | | |
| | | sequence. | | | | |
| 161 | B12117 | F5L15-T7 IGF | 0.54 | A39718 | nicotinic acetylcholine | 0.27 |
| | | Arabidopsis | | | receptor alpha chain - | |
| | | thaliana genomic | | | marbled electric ray | |
| | | clone F5L15. | | | (fragments) | |
| 162 | Z71261 | Caenorhabditis | 0.5 | KDGE_DRO | EYE−SPECIFIC | 4.60E−05 |
| | | elegans cosmid | | ME | DIACYLGLYCEROL | |
| | | F21C3, complete | | | KINASE (EC 2.7.1.107) | |
| | | sequence. | | | (RETINAL | |
| | | | | | DEGENERATION A | |
| | | | | | PROTEIN) | |
| | | | | | (DIGLYCERIDE | |
| | | | | | KINASE) | |
| | | | | | (DGK)>GP:DRODAGK | |
| | | | | | _1 Fruit fly mRNA for | |
| | | | | | diacylglycerol kinase, | |
| | | | | | complete cds | |
| 163 | M61831 | Human S- | 0.49 | P2C2_ARATH | PROTEIN | 5.60E−08 |
| | | adenosylhomocys | | | PHOSPHATASE 2C (EC | |
| | | teine hydrolase | | | 3.1.3.16) | |
| | | (AHCY) mRNA, | | | (PP2C)>PIR2:S55457 | |
| | | complete cds. | | | phosphoprotein | |
| | | | | | phosphatase (EC | |
| | | | | | 3.1.3.16) 2C - | |
| | | | | | Arabidopsis | |
| | | | | | thaliana>GP:ATHPP2CA | |
| | | | | | _1 Arabidopsis thaliana | |
| | | | | | mRNA for protein | |
| | | | | | phosphatase 2C | |
| 164 | U42608 | Glycine max | 0.48 | <NONE> | <NONE> | <NONE> |
| | | clathrin heavy | | | | |
| | | chain mRNA, | | | | |
| | | complete cds. | | | | |
| 165 | Z93042 | Human DNA | 0.47 | PYRD_BACSU | DIHYDROOROTATE | 0.002 |
| | | sequence *** | | | DEHYDROGENASE | |
| | | SEQUENCING | | | (EC 1.3.3.1) | |
| | | IN PROGRESS | | | (DIHYDROOROTATE | |
| | | *** from clone | | | OXIDASE) | |
| | | 6B17; HTGS | | | (DHODEHASE)>PIR1: | |
| | | phase 1. | | | H39845 dihydroorotate | |
| | | | | | oxidase (EC 1.3.3.1) - | |
| | | | | | Bacillus | |
| | | | | | subtilis>GPN:BSUB000 | |
| | | | | | 9_25 Bacillus subtilis | |
| | | | | | complete genome | |
| | | | | | (section 9 of 21): from | |
| | | | | | 1598421 to 1807200; | |
| 166 | AC000044 | Human | 0.47 | MATK_MAR | PROBABLE INTRON | 0.0011 |
| | | Chromosome | | PO | MATURASE>PIR2:A05 | |
| | | 22q13 Cosmid | | | 034 hypothetical protein | |
| | | Clone p76e10, | | | 370i - liverwort | |
| | | complete | | | (Marchantia polymorpha) | |
| | | sequence. | | | chloroplast>GP:CHMPX | |
| | | | | | X_21 Liverwort | |
| | | | | | Marchantia polymorpha | |
| | | | | | chloroplast genome | |
| | | | | | DNA; ORF370i | |
| 167 | X51508 | Rabbit mRNA for | 0.47 | S45361 | LRR47 protein - fruit fly | 5.30E−07 |
| | | aminopeptidase N | | | (Drosophila | |
| | | (partial). | | | melanogaster)>GP:DML | |
| | | | | | RR47_1 D; melanogaster | |
| | | | | | mRNA for LRR47 | |
| 168 | Z67035 | H. sapiens DNA | 0.45 | JQ2246 | 22.5K cathepsin D | 0.79 |
| | | segment | | | inhibitor protein | |
| | | containing (CA) | | | precursor - | |
| | | repeat; clone | | | potato>GP:POTCATHD | |
| | | AFM323yf1; | | | _1 Potato cathepsin D | |
| | | single read. | | | inhibitor protein mRNA, | |
| | | | | | complete cds | |
| 169 | Z93042 | Human DNA | 0.44 | SMU31768_1 | Schistosoma mansoni | 0.0022 |
| | | sequence *** | | | elastase gene, 3045 bp | |
| | | SEQUENCING | | | clone, complete cds | |
| | | IN PROGRESS | | | | |
| | | *** from clone | | | | |
| | | 6B17; HTGS | | | | |
| | | phase 1. | | | | |
| 170 | L11172 | Plasmodium | 0.43 | HUMPKD1G0 | Homo sapiens polycystic | 1 |
| | | falciparum RNA | | 8_1 | kidney disease (PKD1) | |
| | | polymerase I | | | gene, exons 43-46; | |
| | | gene, complete | | | Polycystic kidney disease | |
| | | cds. | | | 1 protein | |
| 171 | Z95889 | Human DNA | 0.43 | A09811_1 | R; norvegicus mRNA for | 0.00083 |
| | | sequence *** | | | BRL-3A binding protein; | |
| | | SEQUENCING | | | Author-given protein | |
| | | IN PROGRESS | | | sequence is in conflict | |
| | | *** from clone | | | with the conceptual | |
| | | 211A9; HTGS | | | translation | |
| | | phase 1. | | | | |
| 172 | U32772 | Haemophilus | 0.43 | YPT2_CAEEL | HYPOTHETICAL 21.6 | 2.50E−28 |
| | | influenzae from | | | KD PROTEIN F37A4.2 | |
| | | bases 954819 to | | | IN CHROMOSOME | |
| | | 966363 (section | | | III>PIR2:S44639 | |
| | | 87 of 163) of the | | | F37A4.2 protein - | |
| | | complete | | | Caenorhabditis | |
| | | genome. | | | elegans >GP:CELF37A4_{—} | |
| | | | | | 8 Caenorhabditis elegans | |
| | | | | | cosmid F37A4 | |
| 173 | Z99281 | Caenorhabditis | 0.42 | PTU19464_1 | Paramecium tetraurelia | 1 |
| | | elegans cosmid | | | outer arm dynein beta | |
| | | Y57G11C, | | | heavy chain gene, | |
| | | complete | | | complete cds | |
| | | sequence. | | | | |
| 174 | X04571 | Human mRNA | 0.42 | YEK9_YEAST | HYPOTHETICAL 53.9 | 0.99 |
| | | for kidney | | | KD PROTEIN IN AFG3- | |
| | | epidermal growth | | | SEB2 INTERGENIC | |
| | | factor (EGF) | | | REGION>PIR2:S50477 | |
| | | precursor. | | | hypothetical protein | |
| | | | | | YER019w - yeast | |
| | | | | | (Saccharomyces | |
| | | | | | cerevisiae)>GP:SCE9537 | |
| | | | | | _20 Saccharomyces | |
| | | | | | cerevisiae chromosome | |
| | | | | | V cosmids 9537, 9581, | |
| | | | | | 9495, 9867, and lambda | |
| | | | | | clone 5898 | |
| 175 | U32772 | Haemophilus | 0.41 | YPT2_CAEEL | HYPOTHETICAL 21.6 | 7.80E−21 |
| | | influenzae from | | | KD PROTEIN F37A4.2 | |
| | | bases 954819 to | | | IN CHROMOSOME | |
| | | 966363 (section | | | III>PIR2:S44639 | |
| | | 87 of 163) of the | | | F37A4.2 protein - | |
| | | complete | | | Caenorhabditis | |
| | | genome. | | | elegans>GP:CELF37A4_{—} | |
| | | | | | 8 Caenorhabditis elegans | |
| | | | | | cosmid F37A4 | |
| 176 | AC002053 | Human | 0.4 | HSU33837_1 | Human glycoprotein | 1 |
| | | Chromosome | | | receptor gp330 precursor, | |
| | | 9p22 Cosmid | | | mRNA, complete cds | |
| | | Clone 92f5, | | | | |
| | | complete | | | | |
| | | sequence. | | | | |
| 177 | U88309 | Caenorhabditis | 0.4 | DROMTTGN | Drosophila melanogaster | 0.99 |
| | | elegans cosmid | | C_1 | mitochondrial | |
| | | T23B3. | | | cytochrome c oxidase | |
| | | | | | subunit I (COI) gene, 5′ | |
| | | | | | end, Trp-, Cys-, and Tyr- | |
| | | | | | tRNA genes, NADH | |
| | | | | | dehydrogenase subunit 2 | |
| | | | | | (ND2) gene, 3′ end | |
| 178 | M34025 | Human fetal Ig | 0.39 | DNA2_YEAST | DNA REPLICATION | 1 |
| | | heavy chain | | | HELICASE | |
| | | variable region | | | DNA2>PIR2:S48904 | |
| | | (clone M44) | | | probable purine | |
| | | mRNA, partial | | | nucleotide-binding | |
| | | cds. | | | protein YHR164c - yeast | |
| | | | | | (Saccharomyces | |
| | | | | | cerevisiae)>GPN:YSCH9 | |
| | | | | | 986_3 Saccharomyces | |
| | | | | | cerevisiae chromosome | |
| | | | | | VIII cosmid 9986; | |
| | | | | | Dna2p: DNA replication | |
| | | | | | helicase; YHR164C>GP: | |
| 179 | AC002395 | Homo sapiens ; | 0.39 | VV_MUMPE | NONSTRUCTURAL | 0.11 |
| | | HTGS phase 1, | | | PROTEIN V | |
| | | 127 unordered | | | (NONSTRUCTURAL | |
| | | pieces. | | | PROTEIN NS1) | |
| 180 | AC003101 | *** | 0.39 | YLK2_CAEEL | HYPOTHETICAL 122.7 | 0.0001 |
| | | SEQUENCING | | | KD PROTEIN D1044.2 | |
| | | IN PROGRESS | | | IN CHROMOSOME | |
| | | *** Homo | | | III>GP:CELD1044_4 | |
| | | sapiens | | | Caenorhabditis elegans | |
| | | chromosome 17, | | | cosmid D1044 | |
| | | clone | | | | |
| | | HRPC41C23; | | | | |
| | | HTGS phase 1, | | | | |
| | | 33 unordered | | | | |
| | | pieces. | | | | |
| 181 | Z54335 | Human DNA | 0.39 | HUMNFAT3 | Homo sapiens NF-AT3 | 1.60E−06 |
| | | sequence from | | A_1 | mRNA, complete cds | |
| | | cosmid L17A9, | | | | |
| | | Huntington's | | | | |
| | | Disease Region, | | | | |
| | | chromosome | | | | |
| | | 4p16.3. Contains | | | | |
| | | VNTR and a CpG | | | | |
| | | island. | | | | |
| 182 | U95743 | Homo sapiens | 0.38 | CEZC434_6 | Caenorhabditis elegans | 0.18 |
| | | chromosome 16 | | | cosmid ZC434, complete | |
| | | BAC clone | | | sequence; ZC434; 6; | |
| | | CIT987-SK65D3, | | | CDNA EST CEESO02F | |
| | | complete | | | comes from this gene; | |
| | | sequence. | | | cDNA EST CEESS60F | |
| | | | | | comes from this gene | |
| 183 | AC001229 | Sequence of BAC | 0.34 | HSOCAM_1 | H; sapiens mRNA for | 0.051 |
| | | F5I14 from | | | immunoglobulin-like | |
| | | Arabidopsis | | | domain-containing 1 | |
| | | thaliana | | | protein | |
| | | chromosome 1, | | | | |
| | | complete | | | | |
| | | sequence. | | | | |
| 184 | X01703 | Human gene for | 0.33 | NTC3_MOUSE | NEUROGENIC LOCUS | 0.012 |
| | | alpha-tubulin (b | | | NOTCH 3 | |
| | | alpha 1). | | | PROTEIN>PIR2:S45306 | |
| | | | | | notch 3 protein - | |
| | | | | | mouse>GP:MMNOTC_1 | |
| | | | | | M; musculus mRNA for | |
| | | | | | Notch 3 | |
| 185 | Z82189 | Human DNA | 0.31 | LG106_3 | Lemna gibba negatively | 0.27 |
| | | sequence *** | | | light-regulated mRNA | |
| | | SEQUENCING | | | (Lg106); Second longest | |
| | | IN PROGRESS | | | ORF (2) | |
| | | *** from clone | | | | |
| | | 170A21; HTGS | | | | |
| | | phase 1. | | | | |
| 186 | Z98051 | Human DNA | 0.3 | S34960 | NADH dehydrogenase | 0.25 |
| | | sequence *** | | | (ubiquinone) (EC | |
| | | SEQUENCING | | | 1.6.5.3) chain 5 - | |
| | | IN PROGRESS | | | Crithidia oncopelti | |
| | | *** from clone | | | mitochondrion | |
| | | 501A4; HTGS | | | (SGC6)>GP:MICOCNN | |
| | | phase 1. | | | R_3 Crithidia oncopelti | |
| | | | | | mitochondrial ND4, | |
| | | | | | ND5, COI, 12S | |
| | | | | | ribosomal RNA genes for | |
| | | | | | NADH dehydrogenase | |
| | | | | | subunit 4/5, cytochrome | |
| | | | | | oxidase subun | |
| 187 | Z98749 | Human DNA | 0.3 | SCKC_LEIQH | CHARYBDOTOXIN | 0.12 |
| | | sequence *** | | | (CHTX) (CHTX- | |
| | | SEQUENCING | | | LQ1)>PIR2:A60963 | |
| | | IN PROGRESS | | | charybdotoxin 1 - | |
| | | *** from clone | | | scorpion (Leiurus | |
| | | 449O17; HTGS | | | quinquestriatus)>3D:2CR | |
| | | phase 1. | | | D Charybdotoxin (nmr, | |
| | | | | | 12 structures) - scorpion | |
| | | | | | (Leiurus quinquestriatus) | |
| 188 | X96763 | C. albicans | 0.29 | CECC4_1 | Caenorhabditis elegans | 1.30E−17 |
| | | CDC4 gene. | | | cosmid CC4, complete | |
| | | | | | sequence; CC4; a; Protein | |
| | | | | | predicted using | |
| | | | | | Genefinder; preliminary | |
| | | | | | prediction | |
| 189 | U38804 | Porphyra | 0.28 | HIVHCDR3C | Human | 1 |
| | | purpurea | | _1 | immunodeficiency virus | |
| | | chloroplast | | | type 1 heavy-chain | |
| | | genome, | | | complemetarity- | |
| | | complete | | | determining region 3 | |
| | | sequence. | | | mRNA (clone 11), partial | |
| | | | | | cds; Heavy-chain | |
| | | | | | complementarity- | |
| | | | | | determining region 3 | |
| | | | | | (CDR3) from IIIV | |
| | | | | | gp120- | |
| | | | | | >GP:HIVHCDR3I_1 | |
| | | | | | Human | |
| | | | | | immunodeficiency virus | |
| | | | | | type 1 he | |
| 190 | U20657 | Human ubiquitin | 0.28 | HSU20657_1 | Human ubiquitin | 5.60E−12 |
| | | protease (Unph) | | | protease (Unph) proto- | |
| | | proto-oncogene | | | oncogene mRNA, | |
| | | mRNA, complete | | | complete cds | |
| | | cds. | | | | |
| 191 | AC002037 | Human | 0.27 | VRP1_YEAST | VERPROLIN>GP:SCVE | 2.00E−11 |
| | | Chromosome 11 | | | RPRL_1 S; cerevisiae | |
| | | Overlapping | | | (A364) gene for | |
| | | Cosmids | | | verprolin | |
| | | cSRL72g7 and | | | | |
| | | cSRL140b8, | | | | |
| | | complete | | | | |
| | | sequence. | | | | |
| 192 | U58748 | Caenorhabditis | 0.27 | EXLP_TOBAC | PISTIL-SECIFIC | 4.10E−12 |
| | | elegans cosmid | | | EXTENSIN-LIKE | |
| | | ZK180. | | | PROTEIN PRECURSOR | |
| | | | | | (PELP)>PIR2:JQ1696 | |
| | | | | | pistil extensin-like | |
| | | | | | protein precursor (clone | |
| | | | | | pMG 15) - common | |
| | | | | | tobacco>GP:NTPMG15_{—} | |
| | | | | | 1 N; tabacum mRNA for | |
| | | | | | pistil extensin like | |
| | | | | | protein | |
| 193 | Z68013 | Caenorhabditis | 0.26 | <NONE> | <NONE> | <NONE> |
| | | elegans cosmid | | | | |
| | | W02H3, | | | | |
| | | complete | | | | |
| | | sequence. | | | | |
| 194 | AF017042 | Dictyostelium | 0.26 | SPBC31F10_14 | S; pombe chromosome II | 1 |
| | | discoideum LTR- | | | cosmid c31F10; | |
| | | retrotransposon | | | Hypothetical protein; | |
| | | Skipper, partial | | | SPBC31F10; 14c, | |
| | | genomic | | | unknown, len:1586aa, | |
| | | sequence, 5′ end. | | | some similarity eg; to | |
| | | | | | YJR140C, | |
| | | | | | YJ9H_YEAST, P47171, | |
| | | | | | involved in cell cycle | |
| | | | | | regulation | |
| 195 | B03174 | cSRL-16e2-u | 0.26 | CELC30E1_7 | Caenorhabditis elegans | 0.38 |
| | | cSRL flow sorted | | | cosmid C30E1 | |
| | | Chromosome 11 | | | | |
| | | specific cosmid | | | | |
| | | Homo sapiens | | | | |
| | | genomic clone | | | | |
| | | cSRL-16e2. | | | | |
| 196 | X70810 | E. gracilis | 0.25 | CEK10H10_8 | Caenorhabditis elegans | 0.98 |
| | | chloroplast | | | cosmid K10H10, | |
| | | complete | | | complete sequence; | |
| | | genome. | | | K10H10; k; Protein | |
| | | | | | predicted using | |
| | | | | | Genefinder; preliminary | |
| | | | | | prediction | |
| 197 | U80024 | Caenorhabditis | 0.25 | MMAF001794 | Mus musculus Treacher | 0.017 |
| | | elegans cosmid | | _1 | Collins Syndrome protein | |
| | | C18B10. | | | (Tcof1) mRNA, | |
| | | | | | complete cds; Putative | |
| | | | | | nucleolar | |
| | | | | | phosphoprotein; similar | |
| | | | | | to Homo sapiens | |
| | | | | | Treacher Collins | |
| | | | | | syndrome TCOF1 protein | |
| | | | | | encoded>GP:MMAF001 | |
| | | | | | 794_1 Mus musculus | |
| | | | | | Treacher Collins | |
| | | | | | Syndrome p | |
| 198 | AC000591 | Drosophila | 0.25 | YHGE_ECOLI | HYPOTHETICAL 64.6 | 0.00068 |
| | | melanogaster | | | KD PROTEIN IN | |
| | | (subclone 9_g3 | | | MRCA-PCKA | |
| | | from P1 DS01486 | | | INTERGENIC REGION | |
| | | (D32)) DNA | | | (F574)>PIR2:E65135 | |
| | | sequence, | | | hypothetical 64.6 kD | |
| | | complete | | | protein in mrcA-pckA | |
| | | sequence. | | | intergenic region - | |
| | | | | | Escherichia coli (strain | |
| | | | | | K- | |
| | | | | | 12)>GP:ECAE000415_7 | |
| | | | | | Escherichia coli, mrcA, | |
| | | | | | yrfE, yrfF, yrfG, yrfH, | |
| | | | | | yrfI | |
| 199 | AC000591 | Drosophila | 0.25 | YHGE_ECOLI | HYPOTHETICAL 64.6 | 0.00068 |
| | | melanogaster | | | KD PROTEIN IN | |
| | | (subclone 9_g3 | | | MRCA-PCKA | |
| | | from P1 DS01486 | | | INTERGENIC REGION | |
| | | (D32)) DNA | | | (F574)>PIR2:E65135 | |
| | | sequence, | | | hypothetical 64.6 kD | |
| | | complete | | | protein in mrcA-pckA | |
| | | sequence. | | | intergenic region - | |
| | | | | | Escherichia coli (strain | |
| | | | | | K- | |
| | | | | | 12)>GP:ECAE000415_7 | |
| | | | | | Escherichia coli, mrcA, | |
| | | | | | yrfE, yrfF, yrfG, yrfH, | |
| | | | | | yrfI | |
| 200 | Z99571 | Human DNA | 0.24 | YA53_SCHPO | HYPOTHETICAL 24.2 | 0.017 |
| | | sequence *** | | | KD PROTEIN | |
| | | SEQUENCING | | | C13A11.03 IN | |
| | | IN PROGRESS | | | CHROMOSOME | |
| | | *** from clone | | | I>GP:SPAC13A11_3 | |
| | | 388N15; HTGS | | | S; pombe chromosome I | |
| | | phase 1. | | | cosmid c13A11; | |
| | | | | | Unknown; | |
| | | | | | SPAC13A11; 03, | |
| | | | | | unknown, len: 210 | |
| 201 | U00672 | Human | 0.24 | TFDP00900 | - Polypeptides entry for | 1.00E−05 |
| | | interleukin-10 | | | factor Oct-2.5 | |
| | | receptor mRNA, | | | | |
| | | complete cds. | | | | |
| 202 | AC003061 | *** | 0.23 | CG1_HUMAN | CG1 | 0.00078 |
| | | SEQUENCING | | | PROTEIN>GP:HSU4602 | |
| | | IN PROGRESS | | | 3_1 Human Xq28 | |
| | | *** Mouse | | | mRNA, complete cds; | |
| | | Chromosome 6 | | | Orf | |
| | | BAC clone | | | | |
| | | b245c12; HTGS | | | | |
| | | phase 2, 8 | | | | |
| | | ordered pieces. | | | | |
| 203 | AF009420 | Homo sapiens | 0.22 | PN0675 | collagen alpha 1(X VIII) | 0.00072 |
| | | microsatellite | | | chain - mouse | |
| | | sequence in the | | | (fragment)>GP:MUSCO | |
| | | HNF3a gene. | | | LLAG_1 Mouse mRNA | |
| | | | | | for collagen, partial cds | |
| 204 | B18861 | F20C18-Sp6 IGF | 0.22 | TFDP00659 | - Polypeptides entry for | 0.0003 |
| | | Arabidopsis | | | factor PR | |
| | | thaliangenomic | | | | |
| | | clone F20C18. | | | | |
| 205 | U00672 | Human | 0.22 | TFDP00900 | - Polypeptides entry for | 1.00E−05 |
| | | interleukin-10 | | | factor Oct-2.5 | |
| | | receptor mRNA, | | | | |
| | | complete cds. | | | | |
| 206 | X52105 | Dictyostelium | 0.18 | <NONE> | <NONE> | <NONE> |
| | | discoideum SP60 | | | | |
| | | gene for spore | | | | |
| | | coat protein. | | | | |
| 207 | L07628 | Saccharopolyspor | 0.17 | D88764_1 | Rana catesbeiana mRNA | 0.00021 |
| | | a erythraea | | | for alpha 2 type I | |
| | | insertion | | | collagen, complete cds | |
| | | sequence IS1136, | | | | |
| | | copy B, 3′ end. | | | | |
| 208 | Z49631 | S. cerevisiae | 0.16 | YSCDAL1A_1 | Saccharomyces | 1 |
| | | chromosome X | | | cerevisiae alantoinase | |
| | | reading frame | | | (DAL1) gene, complete | |
| | | ORF YJR131w. | | | cds | |
| 209 | Z87893 | F. rubripes GSS | 0.16 | CELC27A12_8 | Caenorhabditis elegans | 1.30E−07 |
| | | sequence, clone | | | cosmid C27A12; Partial | |
| | | 043C17aB8. | | | CDS; this gene begins in | |
| | | | | | the neighboring clone; | |
| | | | | | coded for by C; elegans | |
| | | | | | cDNA yk127f1; 3; coded | |
| | | | | | for by C; elegans cDNA | |
| | | | | | yk127f1; 5 | |
| 210 | U92852 | Rhoiptelea | 0.15 | SEU40259_5 | Staphyloccous | 0.95 |
| | | chiliantha | | | epidermidis trimethoprim | |
| | | maturase (matK) | | | resistance plasmid | |
| | | gene, chloroplast | | | pSK639; Orf53 | |
| | | gene encoding | | | | |
| | | chloroplast | | | | |
| | | protein, complete | | | | |
| | | cds. | | | | |
| 211 | X62620 | B. mori Abd-A | 0.15 | ATAP22_36 | Arabidopsis thaliana | 0.75 |
| | | gene homeobox. | | | DNA chromosome 4, | |
| | | | | | ESSA 1 AP2 contig | |
| | | | | | fragment No; 2; | |
| | | | | | Hypothetical protein; | |
| | | | | | Similarity to NADH | |
| | | | | | dehydrogenase, | |
| | | | | | Chondrus crispus; | |
| | | | | | MNOS:S59107 | |
| 212 | J02079 | epstein-barr virus | 0.15 | A38346 | ultra-high-sulfur keratin | 7.50E−05 |
| | | simple repeat | | | 1 - | |
| | | array (ir3). | | | mouse>GP:MUSSER1_1 | |
| | | | | | Mouse serine 1 ultra high | |
| | | | | | sulfur protein gene, | |
| | | | | | complete cds; Putative | |
| 213 | M35027 | Vaccinia virus, | 0.14 | MTF1_FUSNU | MODIFICATION | 0.87 |
| | | complete | | | METHYLASE FNUDI | |
| | | genome. | | | (EC 2.1.1.73) | |
| | | | | | (CYTOSINE−SPECIFIC | |
| | | | | | METHYLTRANSFERA | |
| | | | | | SE FNUDI) (M. FNUDI) | |
| 214 | AC003058 | *** | 0.14 | HEXA_DICDI | BETA- | 0.006 |
| | | SEQUENCING | | | HEXOSAMINIDASE | |
| | | IN PROGRESS | | | ALPHA CHAIN | |
| | | *** Arabidopsis | | | PRECURSOR (EC | |
| | | thaliana ‘IGF’ | | | 3.2.1.52) (N-ACETYL- | |
| | | BAC ‘F27F23’ | | | BETA- | |
| | | genomic | | | GLUCOSAMINIDASE) | |
| | | sequence near | | | (BETA-N- | |
| | | marker | | | ACETYLHEXOSAMINI | |
| | | ‘CIC06E08’; | | | DASE)>PIR2:A30766 | |
| | | HTGS phase 1, 8 | | | beta-N- | |
| | | unordered pieces. | | | acetylhexosaminidase | |
| | | | | | (EC 3.2.1.52) A | |
| | | | | | precursor - slime mold | |
| | | | | | (Dictyostelium | |
| | | | | | discoideum)>GP:DDINA | |
| | | | | | GA_1 D; d | |
| 215 | AC001229 | Sequence of BAC | 0.13 | A49281 | pol protein - simian T- | 0.77 |
| | | F5I14 from | | | cell lymphotropic virus | |
| | | Arabidopsis | | | type 1, STLV-1 (isolate | |
| | | thaliana | | | Bab34) | |
| | | chromosome 1, | | | (fragment)>GP:STVBAB | |
| | | complete | | | POLA_1 Simian T-cell | |
| | | sequence. | | | leukemia virus PCR | |
| | | | | | derived (pol) gene, | |
| | | | | | partial sequence | |
| | | | | | BAB34POL; Bases | |
| | | | | | 4779-4918 EMBL ATK | |
| | | | | | numbering system; | |
| | | | | | BAB34POL | |
| 216 | U46067 | Capra hircus | 0.12 | S70663 | lectin heavy chain, N- | 0.8 |
| | | beta-mannosidase | | | acetylgalactosamine− | |
| | | mRNA, complete | | | specific - Entamoeba | |
| | | cds. | | | histolytica | |
| | | | | | (fragment)>GP:EHU334 | |
| | | | | | 43_1 Entamoeba | |
| | | | | | histolytica GalNAc lectin | |
| | | | | | heavy subunit (hgl4) | |
| | | | | | gene, partial cds; N- | |
| | | | | | acetylgalactosamine | |
| | | | | | adherence lectin heavy | |
| | | | | | subunit | |
| 217 | AC000380 | *** | 0.12 | ATFCA8_19 | Arabidopsis thaliana | 0.64 |
| | | SEQUENCING | | | DNA chromosome 4, | |
| | | IN PROGRESS | | | ESSA I contig fragment | |
| | | *** Human | | | No; 8; Unnamed protein | |
| | | Chromosome 3 | | | product | |
| | | pac pDJ70i11; | | | | |
| | | HTGS phase 1, 2 | | | | |
| | | unordered pieces. | | | | |
| 218 | X61207 | A. brasilense | 0.12 | OCCLO2_1 | O; circumcincta colost-2 | 0.0074 |
| | | hisB, H, A, F | | | gene; Cuticular collagen | |
| | | and E genes for | | | | |
| | | imidazole | | | | |
| | | glycerolphosphat | | | | |
| | | e dehydratase, | | | | |
| | | glutamine | | | | |
| | | amidotransferase, | | | | |
| | | phosphorybosilfo | | | | |
| | | rmimino-5- | | | | |
| | | amino- | | | | |
| | | phosphorybosil- | | | | |
| | | 4- | | | | |
| | | imidazolecarboxa | | | | |
| | | mide isomerase, | | | | |
| | | cyclase and | | | | |
| | | phosphorybosil- | | | | |
| | | AMP- | | | | |
| | | cyclohydrolase. | | | | |
| 219 | AF014259 | HIV-1 Patient | 0.11 | DMU88570_1 | Drosophila melanogaster | 1 |
| | | 1088 from | | | CREB-binding protein | |
| | | Edinburgh, MA- | | | homolog mRNA, | |
| | | p17 (gag) gene, | | | complete cds; CBP | |
| | | partial cds. | | | | |
| 220 | AC000636 | Drosophila | 0.11 | A64829 | hypothetical protein in | 0.051 |
| | | melanogaster | | | dmsC 3′ region- | |
| | | (subclone 2_c11 | | | Escherichia coli (strain | |
| | | from P1 DS07660 | | | K- | |
| | | (D44)) DNA | | | 12)>GP:ECAE000192_1 | |
| | | sequence, | | | Escherichia coli, ycaD, | |
| | | complete | | | ycaK, pflA, pflB, focA | |
| | | sequence. | | | genes from bases 944908 | |
| | | | | | to 955952 (section 82 of | |
| | | | | | 400) of the complete | |
| | | | | | genome; Hypothetical | |
| | | | | | protein in dmsC | |
| 221 | AC002428 | Human BAC | 0.11 | HSNMYC2_1 | Human N-myc gene exon | 0.00014 |
| | | clone GS039E22 | | | 2; Put; N-myc protein (aa | |
| | | from 5q31, | | | 1-263) (953 is 1st base in | |
| | | complete | | | codon) | |
| | | sequence. | | | | |
| 222 | L40949 | Homo sapiens | 0.11 | CEUNC93_2 | C; elegans unc-93 gene; | 1.20E−13 |
| | | (clone AT7-5eu) | | | Protein 2 | |
| | | opioid-receptor- | | | | |
| | | like protein | | | | |
| | | mRNA, 5′ end. | | | | |
| 223 | AL008636 | Human DNA | 0.1 | XELCOL2A1 | Xenopus laevis alpha-1 | 2.60E−06 |
| dir | | sequence *** | | A_1 | collagen type II′ mRNA, | |
| | | SEQUENCING | | | complete cds; Alpha-1 | |
| | | IN PROGRESS | | | type II′ collagen | |
| | | *** from clone | | | | |
| | | 722E9; HTGS | | | | |
| | | phase 1. | | | | |
| 224 | D86993 | Human (lambda) | 0.1 | CELM02B7_2 | Caenorhabditis elegans | 1.80E−09 |
| | | DNA for | | | cosmid M02B7 | |
| | | immunoglobulin | | | | |
| | | light chain. | | | | |
| 225 | AC002539 | Homo sapiens | 0.098 | MTCY7D11_{—} | Mycobacterium | 0.026 |
| | | chromosome 17, | | 17 | tuberculosis cosmid | |
| | | clone 195o20, | | | Y7D11; Unknown; | |
| | | complete | | | MTCY07D11; 17c; | |
| | | sequence. | | | unknown, len: 186 aa, | |
| | | | | | FASTA best: Q10390 | |
| | | | | | Y009_MYCTU | |
| | | | | | hypothetical 31; 0 KD | |
| | | | | | protein MTCY190; 09C | |
| | | | | | (299 aa) opt: 355 z-score: | |
| | | | | | 316; 8 | |
| 226 | M88165 | Human inter- | 0.096 | A54161 | ryanodine−binding | 1 |
| | | alpha-trypsin | | | protein alpha form- | |
| | | inhibitor light | | | bullfrog>GP:D21070_1 | |
| | | chain (ITI) gene, | | | Rana catesbeiana mRNA | |
| | | exon 1. | | | for bullfrog skeletal | |
| | | | | | muscle calcium release | |
| | | | | | channel (ryanodine | |
| | | | | | receptor) alpha | |
| | | | | | isoform(RyR1), complete | |
| | | | | | cds; Ryanodine receptor | |
| | | | | | alpha isoform | |
| 227 | Z92851 | Caenorhabditis | 0.082 | CYA7_BOVIN | ADENYLATE | 0.3 |
| | | elegans DNA *** | | | CYCLASE, TYPE VII | |
| | | SEQUENCING | | | (EC 4.6.1.1) (ATP | |
| | | IN PROGRESS | | | PYROPHOSPHATE− | |
| | | *** from clone | | | LYASE) (ADENYLYL | |
| | | Y39G8; HTGS | | | CYCLASE) | |
| | | phase 1. | | | | |
| 228 | L00638 | Arabidopsis | 0.072 | NUCM_TRY | NADH-UBIQUINONE | 0.24 |
| | | thaliana ubiquitin | | BB | OXIDOREDUCTASE | |
| | | conjugating | | | 49 KD SUBUNIT | |
| | | enzyme exons 2- | | | HOMOLOG (EC 1.6.5.3) | |
| | | 4. | | | (NADH | |
| | | | | | DEHYDROGENASE | |
| | | | | | SUBUNIT 7 | |
| | | | | | HOMOLOG)>PIR2:A35 | |
| | | | | | 693 NADH | |
| | | | | | dehydrogenase (EC | |
| | | | | | 1.6.99.3) chain 7- | |
| | | | | | Trypanosoma brucei | |
| | | | | | mitochondrion (SGC6) | |
| 229 | U49169 | Dictyostelium | 0.071 | MMU65594_1 | Mus musculus Brca2 | 1 |
| | | discoideum V- | | | mRNA, complete cds; | |
| | | ATPase A | | | Similar to human breast | |
| | | subunit (vatA) | | | cancer susceptibility gene | |
| | | mRNA, complete | | | BRCA2; Allele: wild | |
| | | cds. | | | type; putative tumor | |
| | | | | | suppressor | |
| 230 | AF001549 | Homo sapiens | 0.07 | PM22_HUMAN | PERIPHERAL MYELIN | 0.0078 |
| | | chromosome 16 | | | PROTEIN 22 (PMP- | |
| | | BAC clone | | | 22)>PIR2:JN0503 | |
| | | CIT987SK- | | | peripheral myelin protein | |
| | | 270G1 complete | | | 22- | |
| | | sequence. | | | human>GP:HUMGAS3 | |
| | | | | | X_1 Human peripheral | |
| | | | | | myelin protein 22 | |
| | | | | | (GAS3) mRNA, | |
| | | | | | complete | |
| | | | | | cds>GP:HUMPMP22_1 | |
| | | | | | Human peripheral myelin | |
| | | | | | protein 22 mRNA, | |
| | | | | | complete | |
| | | | | | cds>GP:HUMPMP22 | |
| 231 | L36829 | Mus musculus | 0.066 | <NONE> | <NONE> | <NONE> |
| | | alphaA-crystallin- | | | | |
| | | binding protein I | | | | |
| | | (AlphaA- | | | | |
| | | CRYBP1) gene, | | | | |
| | | complete cds. | | | | |
| 232 | AC000159 | *** | 0.058 | CEZK863_1 | Caenorhabditis elegans | 1 |
| | | SEQUENCING | | | cosmid ZK863, complete | |
| | | IN PROGRESS | | | sequence; ZK863; 2; | |
| | | *** Human BAC | | | Similar to collagen | |
| | | Clone 11q13; | | | | |
| | | HTGS phase 1, | | | | |
| | | 10 unordered | | | | |
| | | pieces. | | | | |
| 233 | AC000159 | *** | 0.058 | CAC2_HAECO | CUTICLE COLLAGEN | 1.20E−08 |
| | | SEQUENCING | | | 2C | |
| | | IN PROGRESS | | | (FRAGMENT)>GP:HAE | |
| | | *** Human BAC | | | COL2C_1 H; contortus | |
| | | Clone 11q13; | | | collagen 2C mRNA, | |
| | | HTGS phase 1, | | | 3′ end | |
| | | 10 unordered | | | | |
| | | pieces. | | | | |
| 234 | Z23908 | H. sapiens | 0.057 | VEU34999_1 | Venezuelan equine | 0.0002 |
| | | (D5S630) DNA | | | encephalitis virus | |
| | | segment | | | nonstructural and | |
| | | containing (CA) | | | structural polyprotein | |
| | | repeat; clone | | | genes, complete cds; | |
| | | AFM268zd9; | | | Nonstructural | |
| | | single read. | | | polyprotein; Internal stop | |
| | | | | | codon, readthrough | |
| | | | | | occurs 5% of the time | |
| 235 | B21875 | T3E8-Sp6 TAMU | 0.055 | YRR2_CAEEL | HYPOTHETICAL 91.1 | 0.68 |
| | | Arabidopsis | | | KD PROTEIN R144.2 | |
| | | thaliana genomic | | | IN CHROMOSOME | |
| | | clone T3E8. | | | III>GP:CELR144_7 | |
| | | | | | Caenorhabditis elegans | |
| | | | | | cosmid R144; Coded for | |
| | | | | | by C; elegans cDNA | |
| | | | | | CEESP84R; coded for by | |
| | | | | | C; elegans cDNA | |
| | | | | | yk23c4; 5; coded for by | |
| | | | | | C; elegans cDNA | |
| | | | | | yk44f9; 5; coded for by | |
| | | | | | C; eleg | |
| 236 | Z98303 | Human DNA | 0.048 | AC002330_3 | Arabidopsis thaliana | 0.99 |
| | | sequence *** | | | BAC T10P11, complete | |
| | | SEQUENCING | | | sequence; Putative zinc- | |
| | | IN PROGRESS | | | finger protein; C2H2 Zn- | |
| | | *** from clone | | | finger signature from | |
| | | 140H19; HTGS | | | position 80 to 100 | |
| | | phase 1. | | | [CEICNKGFQRDQNLQ | |
| | | | | | LHRRGH] | |
| 237 | D49911 | Thermus | 0.044 | APP1_MOUSE | AMYLOID-LIKE | 8.90E−06 |
| | | thermophilus | | | PROTEIN 1 | |
| | | UvrA gene, | | | PRECURSOR | |
| | | complete cds. | | | (APLP)>PIR2:A46362 | |
| | | | | | amyloid precursor-like | |
| | | | | | protein- | |
| | | | | | mouse>GP:MUSAPLP_{—} | |
| | | | | | 1 Mouse amyloid | |
| | | | | | precursor-like protein | |
| | | | | | mRNA, complete cds | |
| 238 | D49911 | Thermus | 0.044 | MMCOL18A1 | Mus musculus alpha- | 1.60E−06 |
| | | thermophilus | | 1_2 | 1(XVIII) collagen | |
| | | UvrA gene, | | | (COL18A1) gene, exons | |
| | | complete cds. | | | 40- 43, complete cds | |
| 239 | X78119 | P. amygdalus, | 0.042 | CA44_HUMAN | COLLAGEN ALPHA | 2.00E−06 |
| | | Batsch (Texas) | | | 4(IV) CHAIN | |
| | | pru1 mRNA. | | | PRECURSOR>PIR1:CG | |
| | | | | | HU1B collagen alpha | |
| | | | | | 4(IV) chain precursor - | |
| | | | | | human>GP:HSCOL4A4_{—} | |
| | | | | | 1 H; sapiens mRNA for | |
| | | | | | collagen type IV alpha 4 | |
| | | | | | chain; Type IV collagen | |
| | | | | | alpha 4 chain | |
| 240 | U72877 | Rana catesbeiana | 0.041 | YRR6_MYCCA | HYPOTHETICAL 33.0 | 0.0008 |
| | | L-epinephrine | | | KD PROTEIN IN LICA | |
| | | transporter | | | 3′ REGION (ORF | |
| | | mRNA, complete | | | R6)>PIR2:S42125 | |
| | | cds. | | | hypothetical protein 3 - | |
| | | | | | Mycoplasma capricolum | |
| | | | | | (SGC3)>GP:MYCRPM | |
| | | | | | H_6 M; capricolum | |
| | | | | | rpmH, rnpA and licA | |
| | | | | | gene; Orf R6 | |
| 241 | L39891 | Homo sapiens | 0.04 | MUC2_HUM | MUCIN 2 | 5.90E−05 |
| | | polycystic kidney | | AN | (INTESTINAL MUCIN | |
| | | disease− | | | 2) (FRAGMENTS) | |
| | | associated protein | | | | |
| | | (PKD1) gene, | | | | |
| | | complete cds. | | | | |
| 242 | L40390 | Candida glabrata | 0.039 | G01763 | atrophin-1 - | 9.00E−07 |
| | | ERG3 gene, | | | human>GP:HSU23851_1 | |
| | | complete cds. | | | Human atrophin-1 | |
| | | | | | mRNA, complete cds | |
| 243 | B28113 | T2L16TRB | 0.038 | CELZK1248_{—} | Caenorhabditis elegans | 1.60E−18 |
| | | TAMU | | 14 | cosmid ZK1248 | |
| | | Arabidopsis | | | | |
| | | thaliana genomic | | | | |
| | | clone T2L16. | | | | |
| 244 | AC000030 | 00175, complete | 0.033 | ATFCA8_40 | Arabidopsis thaliana | 0.63 |
| | | sequence. | | | DNA chromosome 4, | |
| | | | | | ESSA I contig fragment | |
| | | | | | No; 8; Glycerol-3- | |
| | | | | | phosphate permease | |
| | | | | | homolog; Similarity to | |
| | | | | | glycerol-3-phosphate | |
| | | | | | permease - Haemophilus | |
| | | | | | influenzae | |
| 245 | B10738 | F13G15-Sp6 IGF | 0.032 | D87521_1 | Mus musculus DNA- | 0.21 |
| | | Arabidopsis | | | PKcs mRNA, complete | |
| | | thaliana genomic | | | cds | |
| | | clone F13G15. | | | | |
| 246 | AF024503 | Caenorhabditis | 0.03 | I38344 | titin - human | 1 |
| | | elegans cosmid | | | | |
| | | F31F4. | | | | |
| 247 | Z49888 | Caenorhabditis | 0.027 | KSU52064_1 | Kaposi's sarcoma- | 3.40E−10 |
| | | elegans cosmid | | | associated herpes-like | |
| | | F47A4, complete | | | virus ORF73 homolog | |
| | | sequence. | | | gene, complete cds; | |
| | | | | | Herpesvirus saimiri | |
| | | | | | ORF73 | |
| | | | | | homolog>GP:KSU75698_{—} | |
| | | | | | 78 Kaposi's sarcoma- | |
| | | | | | associated herpesvirus | |
| | | | | | long unique region, 80 | |
| | | | | | putative ORF's and | |
| | | | | | kaposin gene, complete | |
| | | | | | cds; OR | |
| 248 | Z83822 | Human DNA | 0.025 | GRSB_BACBR | GRAMICIDIN S | 1 |
| | | sequence from | | | SYNTHETASE II | |
| | | PAC 306D1 on | | | (GRAMICIDIN S | |
| | | chromosome X | | | BIOSYNTHESIS GRSB | |
| | | contains ESTs. | | | PROTEIN) (EC 6.-.-.-) | |
| 249 | Z94161 | Human DNA | 0.025 | S16323 | hypothetical protein - | 0.0079 |
| | | sequence *** | | | Arabidopsis | |
| | | SEQUENCING | | | thaliana>GP:ATHB1_1 | |
| | | IN PROGRESS | | | A; thaliana homeobox | |
| | | *** from clone | | | gene Athb-1 mRNA; | |
| | | N102C10; HTGS | | | Open reading frame | |
| | | phase 1. | | | | |
| 250 | AC002094 | Genomic | 0.021 | S57447 | HPBRII-7 protein - | 8.20E−08 |
| | | sequence from | | | human>GP:HSHPBRII4 | |
| | | Human 17, | | | _1 H; sapiens HPBRII-4 | |
| | | complete | | | mRNA>GP:HSHPBRII7 | |
| | | sequence. | | | _1 H; sapiens HPBRII-7 | |
| | | | | | gene | |
| 251 | D79994 | Human mRNA | 0.021 | CER10H10_1 | Caenorhabditis elegans | 7.00E−16 |
| | | for KIAA0172 | | | cosmid R10H10, | |
| | | gene, partial cds. | | | complete sequence; | |
| | | | | | R11A8; 7; Protein | |
| | | | | | predicted using | |
| | | | | | Genefinder; Similarity to | |
| | | | | | Mouse ankyrin (PIR Acc; | |
| | | | | | No; S37771); cDNA EST | |
| | | | | | CEESX25F comes from | |
| | | | | | this gene; | |
| 252 | Z97635 | Human DNA | 0.017 | CELW05H7_4 | Caenorhabditis elegans | 0.24 |
| | | sequence *** | | | cosmid W05H7 | |
| | | SEQUENCING | | | | |
| | | IN PROGRESS | | | | |
| | | *** from clone | | | | |
| | | 438L4; HTGS | | | | |
| | | phase 1. | | | | |
| 253 | X84996 | X. laevis mRNA | 0.017 | JN0786 | integrin beta-4 chain | 0.088 |
| | | for selenocysteine | | | precursor - mouse | |
| | | tRNA acting | | | | |
| | | factor (Staf). | | | | |
| 254 | AC002543 | Human BAC | 0.013 | MZLMTCYT | Mendozellus isis | 0.044 |
| | | clone RG300C03 | | BT_1 | mitochondrial NADH | |
| | | from 7q31.2, | | | dehydrogenase, and | |
| | | complete | | | cytochrome b genes, 3′ | |
| | | sequence. | | | end, and transfer RNA- | |
| | | | | | Ser gene; This codes for | |
| | | | | | the last 43 amino acids of | |
| | | | | | NADH dehydrogenase | |
| | | | | | subunit 1 followed | |
| 255 | U10401 | Caenorhabditis | 0.012 | MMMHC29N | Mus musculus major | 0.069 |
| | | elegans cosmid | | 7_2 | histocompatibility locus | |
| | | T20B12. | | | class III | |
| | | | | | region:butyrophilin-like | |
| | | | | | protein gene, partial cds; | |
| | | | | | Notch4, PBX2, RAGE, | |
| | | | | | lysophatidic acid acyl | |
| | | | | | transferase−alpha, | |
| | | | | | palmitoyl- | |
| 256 | L14593 | Saccharomyces | 0.011 | D86995_1 | Human (gene 1) DNA for | 2.20E−14 |
| | | cerevisiae protein | | | phosphatase 2C motif, | |
| | | phosphatase | | | partial cds | |
| | | (PTC1) gene, | | | | |
| | | complete cds. | | | | |
| 257 | U62317 | Chromosome | 0.0093 | P2Y8_XENLA | P2Y PURINOCEPTOR 8 | 0.89 |
| | | 22q13 BAC | | | (P2Y8)>GP:XLP2Y8_1 | |
| | | Clone | | | X; laevis mRNA for | |
| | | CIT987SK- | | | P2Y8 nucleotide receptor | |
| | | 384D8 complete | | | | |
| | | sequence. | | | | |
| 258 | D29655 | Pig mRNA for | 0.0075 | AF004858_1 | Mus musculus platelet | 1 |
| | | UMP-CMP | | | activating factor receptor | |
| | | kinase, complete | | | mRNA, partial cds; PAF- | |
| | | cds. | | | receptor | |
| 259 | AF002992 | Homo sapiens | 0.0054 | FBN1_BOVIN | FIBRILLIN 1 | 0.0004 |
| | | cosmid from | | | PRECURSOR>PIR2:A5 | |
| | | Xq28, complete | | | 5567 fibrillin I - | |
| | | sequence. | | | bovine>GP:BOVXAAA | |
| | | | | | A_1 Bos taurus mRNA, | |
| | | | | | complete cds; Putative | |
| 260 | B20752 | T19M2-T7 | 0.0043 | HSVT1IEP_1 | Feline herpesvirus type 1 | 3.90E−05 |
| | | TAMU | | | gene for immediate early | |
| | | Arabidopsis | | | protein, complete cds; | |
| | | thaliana genomic | | | Feline herpesvirus type 1 | |
| | | clone T19M2. | | | immediate early protein | |
| 261 | AB006699 | Arabidopsis | 0.0037 | YHV5_YEAST | HYPOTHETICAL 143.6 | 0.077 |
| | | thaliana genomic | | | KD PROTEIN IN | |
| | | DNA, | | | SPO16-REC104 | |
| | | chromosome 5, | | | INTERGENIC | |
| | | P1 clone: MDJ22. | | | REGION>PIR2:S46754 | |
| | | | | | hypothetical protein | |
| | | | | | YHR155w - yeast | |
| | | | | | (Saccharomyces | |
| | | | | | cerevisiae)>GPN:YSCH9 | |
| | | | | | 666_15 Saccharomyces | |
| | | | | | cerevisiae chromosome | |
| | | | | | VIII cosmid 9666; | |
| | | | | | Yhr155wp; Similar to | |
| | | | | | Sip3p (Snf | |
| 262 | Z99128 | Human DNA | 0.0032 | ALU1_HUM | !!!! ALU SUBFAMILY J | 0.0087 |
| | | sequence *** | | AN | WARNING ENTRY !!!! | |
| | | SEQUENCING | | | | |
| | | IN PROGRESS | | | | |
| | | *** from clone | | | | |
| | | 422H11; HTGS | | | | |
| | | phase 1. | | | | |
| 263 | B21848 | T2D2-Sp6 | 0.0031 | B31794 | mdm-1 protein (clone | 1.00E−05 |
| | | TAMU | | | c103) - mouse | |
| | | Arabidopsis | | | | |
| | | thaliana genomic | | | | |
| | | clone T2D2. | | | | |
| 264 | L33853 | Human germline | 0.0027 | B45550 | cytochrome b homolog - | 0.99 |
| | | immunoglobulin | | | Plasmodium yoelii | |
| | | kappa chain | | | | |
| | | variable region | | | | |
| | | (Vk-IV subgroup) | | | | |
| | | for anti-B- | | | | |
| | | amyloid | | | | |
| | | autoantibodies in | | | | |
| | | Alzheimer's | | | | |
| | | disease. | | | | |
| 265 | B36863 | HS-1042-A1- | 0.0027 | YQK4_CAEEL | HYPOTHETICAL 64.3 | 0.81 |
| | | F01-MR.abi CIT | | | KD PROTEIN C56G2.4 | |
| | | Human Genomic | | | IN CHROMOSOME | |
| | | Sperm Library C | | | III>GP:CELC56G2_2 | |
| | | Homo sapiens | | | Caenorhabditis elegans | |
| | | genomic clone | | | cosmid C56G2 | |
| | | Plate = CT 824 | | | | |
| | | Col = 1 Row = K. | | | | |
| 266 | AC003041 | *** | 0.0024 | GLB4_LAMSP | GIANT HEMOGLOBIN | 0.94 |
| | | SEQUENCING | | | AIV CHAIN | |
| | | IN PROGRESS | | | (FRAGMENT)>PIR2:S0 | |
| | | *** Homo | | | 1810 hemoglobin AIV - | |
| | | sapiens | | | tube worm | |
| | | chromosome 17, | | | (Lamellibrachia sp.) | |
| | | clone | | | (fragment) | |
| | | HCIT307A16; | | | | |
| | | HTGS phase 1, | | | | |
| | | 10 unordered | | | | |
| | | pieces. | | | | |
| 267 | AC002315 | Mouse BAC- | 0.0022 | MG42_TARMA | SRY-RELATED | 0.99 |
| | | 146N21 | | | PROTEIN MG42 | |
| | | Chromosome X | | | (FRAGMENT)>PIR3:I5 | |
| | | contains | | | 1369 Sry-related | |
| | | iduronate−2- | | | sequence - Tarentola | |
| | | sulfatase gene; | | | mauritanica | |
| | | complete | | | (fragment)>GP:TELMG4 | |
| | | sequence. | | | 2DNA_1 Gecko MG42 | |
| | | | | | gene, partial cds; Sry- | |
| | | | | | related sequence | |
| 268 | AF016674 | Caenorhabditis | 0.0015 | SCYJL204C_1 | S; cerevisiae chromosome | 1 |
| | | elegans cosmid | | | X reading frame ORF | |
| | | C03H5. | | | YJL204c | |
| 269 | AF016674 | Caenorhabditis | 0.0015 | CEM199_3 | Caenorhabditis elegans | 0.97 |
| | | elegans cosmid | | | cosmid M199, complete | |
| | | C03H5. | | | sequence; M199; e; | |
| | | | | | Protein predicted using | |
| | | | | | Genefinder; preliminary | |
| | | | | | prediction | |
| 270 | AF016674 | Caenorhabditis | 0.0015 | CEM199_3 | Caenorhabditis elegans | 0.97 |
| | | elegans cosmid | | | cosmid M199, complete | |
| | | C03H5. | | | sequence; M199; e; | |
| | | | | | Protein predicted using | |
| | | | | | Genefinder; preliminary | |
| | | | | | prediction | |
| 271 | Z54199 | L. esculentum | 0.0015 | CELF20A1_5 | Caenorhabditis elegans | 0.11 |
| | | DNA Ailsa craig | | | cosmid F20A1; Coded | |
| | | encoding 1- | | | for by C; elegans cDNA | |
| | | aminocyclopropa | | | yk9g1; 3; coded for by C; | |
| | | ne−1-carboxylic | | | elegans cDNA yk9g1; 5; | |
| | | acid oxidase. | | | coded for by C; elegans | |
| | | | | | cDNA CEESU55F; weak | |
| | | | | | similarity to putative | |
| 272 | Z99943 | Human DNA | 0.0014 | CEK08F8_5 | Caenorhabditis elegans | 0.93 |
| | | sequence *** | | | cosmid K08F8, complete | |
| | | SEQUENCING | | | sequence; K08F8; 5b | |
| | | IN PROGRESS | | | | |
| | | *** from clone | | | | |
| | | 313L4; HTGS | | | | |
| | | phase 1. | | | | |
| 273 | S81083 | beta- | 0.0013 | MTCY277_7 | Mycobacterium | 0.0001 |
| | | ADD = adducin | | | tuberculosis cosmid | |
| | | beta subunit 63 | | | Y277; Unknown; | |
| | | kda | | | MTCY277; 07c, | |
| | | isoform/membran | | | unknown, len: 302 | |
| | | e skeleton | | | | |
| | | protein, beta - | | | | |
| | | ADD'2 adducin | | | | |
| | | beta subunit 63 | | | | |
| | | kda | | | | |
| | | isoform/membran | | | | |
| | | e skeleton protein | | | | |
| | | {alternatively | | | | |
| | | spliced, exon 10 | | | | |
| | | to 13 region} | | | | |
| | | [human, | | | | |
| | | Genomic, 1851 | | | | |
| | | nt, segment 3 of | | | | |
| | | 3]. | | | | |
| 274 | Z82174 | Human DNA | 0.001 | FBLA_HUM | FIBULIN-1, ISOFORM | 0.00063 |
| | | sequence from | | AN | A | |
| | | cosmid B20F6 on | | | PRECURSOR>GP:HSFI | |
| | | chromosome | | | BUA_1 H; sapiens | |
| | | 22q11.2-qter. | | | mRNA for fibulin-1 A | |
| 275 | Z82215 | Human DNA | 0.00079 | BFR1_SCHPO | BREFELDIN A | 0.15 |
| | | sequence *** | | | RESISTANCE | |
| | | SEQUENCING | | | PROTEIN>PIR2:S52239 | |
| | | IN PROGRESS | | | hba2 protein - fission | |
| | | *** from clone | | | yeast | |
| | | 68O2; HTGS | | | (Schizosaccharomyces | |
| | | phase 1. | | | pombe)>GP:SPHBA2GE | |
| | | | | | N_1 S; pombe hba2 gene | |
| 276 | U28153 | Caenorhabditis | 0.00071 | CX2_HEMHA | CYTOTOXIN 2 (TOXIN | 0.32 |
| | | elegans UNC-76 | | | 12A) | |
| | | (unc-76) gene, | | | | |
| | | complete cds. | | | | |
| 277 | Z82204 | Human DNA | 0.00054 | DMU34925_2 | Drosophila melanogaster | 0.045 |
| | | sequence from | | | DNA repair protein (mei- | |
| | | clone J362G171. | | | 41) gene, complete cds, | |
| | | | | | and TH1 gene, partial cds | |
| 278 | AC002530 | Human BAC | 0.00053 | CELT28F2_2 | Caenorhabditis elegans | 0.037 |
| | | clone RG341D10 | | | cosmid T28F2; Weak | |
| | | from 7p15-p21, | | | similarity to HSP90 | |
| | | complete | | | | |
| | | sequence. | | | | |
| 279 | U91322 | Human | 0.00051 | CEW08D2_2 | Caenorhabditis elegans | 0.26 |
| | | chromosome | | | cosmid W08D2, | |
| | | 16p13 BAC clone | | | complete sequence; | |
| | | CIT987SK-276F8 | | | W08D2; 3; Protein | |
| | | complete | | | predicted using | |
| | | sequence. | | | Genefinder>GP:CEW08 | |
| | | | | | D2_2 Caenorhabditis | |
| | | | | | elegans cosmid W08D2; | |
| | | | | | W08D2; 3; Protein | |
| | | | | | predicted using | |
| | | | | | Genefinder | |
| 280 | D16986 | Human HepG2 | 0.00037 | POLG_PPVNA | GENOME | 0.48 |
| | | partial cDNA, | | | POLYPROTEIN | |
| | | clone | | | (CONTAINS: N- | |
| | | hmd2b09m5. | | | TERMINAL PROTEIN; | |
| | | | | | HELPER COMPONENT | |
| | | | | | PROTEINASE (EC | |
| | | | | | 3.4.22.-) (HC-PRO); 42- | |
| | | | | | 50 KD PROTEIN; | |
| | | | | | CYTOPLASMIC | |
| | | | | | INCLUSION PROTEIN | |
| | | | | | (CI); 6 KD PROTEIN; | |
| | | | | | NUCLEAR | |
| | | | | | INCLUSION PROTEIN | |
| | | | | | A (NI-A) (EC 3.4.22.-) | |
| | | | | | (49K PROTEINASE) (49 | |
| 281 | U91318 | Human | 0.00031 | <NONE> | <NONE> | <NONE> |
| | | chromosome | | | | |
| | | 16p13 BAC clone | | | | |
| | | CIT987SK- | | | | |
| | | 962B4 complete | | | | |
| | | sequence. | | | | |
| 282 | M93406 | Human dispersed | 0.0003 | VG8_SPV4 | GENE 8 | 0.23 |
| | | Alu repeats and | | | PROTEIN>PIR1:G8BPS | |
| | | dispersed L1 | | | V gene 8 protein - | |
| | | repeat. | | | spiroplasma virus 4 | |
| | | | | | (SGC3) | |
| 283 | AC002398 | Human DNA | 0.00021 | HMCA_DRO | HOMEOTIC CAUDAL | 0.021 |
| | | from | | ME | PROTEIN>PIR2:A26357 | |
| | | chromosome 19- | | | homeotic protein Cad - | |
| | | specific cosmid | | | fruit fly (Drosophila | |
| | | F25965, genomic | | | melanogaster)>GP:DRO | |
| | | sequence, | | | CADA2_1 | |
| | | complete | | | D; melanogaster caudal | |
| | | sequence. | | | gene (cad) encoding a | |
| | | | | | maternal and zygotic | |
| | | | | | transcript, exon 2; Caudal | |
| | | | | | protein>TFD:TFDP0015 | |
| | | | | | 9 - Polypeptides en | |
| 284 | AC002530 | Human BAC | 0.0002 | PL0009 | complement | 0.7 |
| | | clone RG341D10 | | | C3d/Epstein-Barr virus | |
| | | from 7p15-p21, | | | receptor precursor - | |
| | | complete | | | human | |
| | | sequence. | | | | |
| 285 | X01871 | Yeast | 0.00015 | RVZMTCYT | Reventazonia sp; | 0.73 |
| | | mitochondrial | | BT_1 | mitochondrial NADH | |
| | | ori(o) repeat unit | | | dehydrogenase, and | |
| | | of petite mutant 5 | | | cytochrome b genes, 3′ | |
| | | (petite strain s- | | | end, and transfer RNA- | |
| | | 10/7/2). | | | Ser gene; This codes for | |
| | | | | | the last 43 amino acids of | |
| | | | | | NADH dehydrogenase | |
| | | | | | subunit 1 followed | |
| 286 | U89984 | Acanthamoeba | 0.00015 | ACU89984_1 | Acanthamoeba castellanii | 4.20E−13 |
| | | castellanii | | | transformation-sensitive | |
| | | transformation- | | | protein homolog mRNA, | |
| | | sensitive protein | | | complete cds; Similar to | |
| | | homolog mRNA, | | | human transformation- | |
| | | complete cds. | | | sensitive protein: | |
| | | | | | SwissProt Accession | |
| | | | | | Number P31948 | |
| 287 | AC002365 | Homo sapiens | 0.00011 | S10340 | DNA-directed RNA | 0.00062 |
| | | chromosome X | | | polymerase (EC 2.7.7.6) | |
| | | clone U177G4, | | | - yeast (Kluyveromyces | |
| | | U152H5, | | | marxianus var. lactis) | |
| | | U168D5, 174A6, | | | | |
| | | U172D6, and | | | | |
| | | U186B3 from | | | | |
| | | Xp22, complete | | | | |
| | | sequence. | | | | |
| 288 | AC002390 | Human DNA | 9.90E−05 | D86603_1 | Mouse mRNA for Bach | 1 |
| | | from overlapping | | | protein 1, complete cds; | |
| | | chromosome 19- | | | Bach 1 | |
| | | specific cosmids | | | | |
| | | R30072 and | | | | |
| | | R28588, genomic | | | | |
| | | sequence, | | | | |
| | | complete | | | | |
| | | sequence. | | | | |
| 289 | AC002980 | Homo sapiens ; | 9.20E−05 | TRBKPCYB_1 | Trypanosoma brucei | 0.52 |
| | | HTGS phase 1, | | | kinetoplast | |
| | | 34 unordered | | | apocytochrome b gene, | |
| | | pieces. | | | complete cds | |
| 290 | M99412 | Human | 4.50E−05 | S28832 | microtubule−associated | 0.88 |
| | | interleukin-8 | | | protein H1 (clone KS3.1) | |
| | | receptor (IL8RB) | | | - longfin squid | |
| | | gene, complete | | | (fragment) | |
| | | cds. | | | | |
| 291 | AC000120 | Human BAC | 4.00E−05 | SXSCRBA_1 | S; xylosus scrB and scrR | 0.99 |
| | | clone RG161K23 | | | genes; Sucrose repressor | |
| | | from 7q21, | | | | |
| | | complete | | | | |
| | | sequence. | | | | |
| 292 | AC003037 | Homo sapiens; | 3.40E−05 | S13569 | hypothetical protein 5 - | 0.018 |
| | | HTGS phase 1, | | | Lactococcus lactis subsp, | |
| | | 66 unordered | | | lactis insertion sequence | |
| | | pieces. | | | 1076>GP:LLTLE_1 | |
| | | | | | Lactococcus lactis DNA | |
| | | | | | for the transposon-like | |
| | | | | | element on the lactose | |
| | | | | | plasmid; ORF5 (AA 1 - | |
| | | | | | 43) | |
| 293 | Z81512 | Caenorhabditis | 2.40E−05 | MUSDBPRC_1 | Mus musculus DNA- | 1 |
| | | elegans cosmid | | | binding protein Rc | |
| | | F25C8, complete | | | mRNA, complete cds; | |
| | | sequence. | | | DNA binding protein Rc | |
| 294 | B16681 | 343C3.TVB | 1.10E−05 | COPP_YEAST | COATOMER BETA′ | 0.081 |
| | | CIT978SKA1 | | | SUBUNIT (BETA′ - | |
| | | Homo sapiens | | | COAT PROTEIN) | |
| | | genomic clone A- | | | BETA′ - | |
| | | 343C03. | | | COP)>PIR2:B55123 | |
| | | | | | coatomer complex beta′ | |
| | | | | | chain - yeast | |
| | | | | | (Saccharomyces | |
| | | | | | cerevisiae)>GPN:SCYG | |
| | | | | | L137W_1 S; cerevisiae | |
| | | | | | chromosome VII reading | |
| | | | | | frame ORF | |
| | | | | | YGL137w>GP:SCU1123 | |
| | | | | | 7_1 Saccharomyces | |
| | | | | | cerevisiae | |
| 295 | Z16523 | H. sapiens | 1.00E−05 | MMSEMF_1 | M; musculus mRNA for | 0.78 |
| | | (D9S158) DNA | | | semaphorin F; | |
| | | segment | | | Smaphorin F | |
| | | containing (CA) | | | | |
| | | repeat; clone | | | | |
| | | AFM073yb11; | | | | |
| | | single read. | | | | |
| 296 | Z49704 | S. cerevisiae | 5.60E−06 | <NONE> | <NONE> | <NONE> |
| | | chromosome XIII | | | | |
| | | cosmid 8021. | | | | |
| 297 | AC003071 | Human BAC | 3.00E−06 | HSRCAER_1 | H; sapiens mRNA for red | 0.21 |
| | | clone BK085E05 | | | cell anion exchanger | |
| | | from 22q12.1- | | | (EPB3, AE1, Band 3) 3′ | |
| | | qter, complete | | | non-coding region | |
| | | sequence. | | | | |
| 298 | U20428 | Human SNC19 | 1.40E−06 | HUMMUC2A | Human mucin-2 gene, | 4.40E−06 |
| | | mRNA sequence. | | _1 | partial cds | |
| 299 | U51903 | Human RasGAP- | 6.60E−07 | IQGA_HUMAN | RAS GTPASE− | 1.60E−14 |
| | | related protein | | | ACTIVATING-LIKE | |
| | | (IQGAP2) | | | PROTEIN IQGAP1 | |
| | | mRNA, complete | | | (P195)>PIR2:A54854 | |
| | | cds. | | | Ras GTPase activating- | |
| | | | | | related protein - | |
| | | | | | human>GP:HUMIQGA_{—} | |
| | | | | | 1 Homo sapiens ras | |
| | | | | | GTPase−activating-like | |
| | | | | | protein (IQGAP1) | |
| | | | | | mRNA, complete cds; | |
| | | | | | Amino acid feature: IQ | |
| | | | | | calmodulin-binding do | |
| 300 | AL000805 | F. rubripes GSS | 4.70E−07 | MT13_MYTED | METALLOTHIONEIN | 2.20E−10 |
| | | sequence, clone | | | 10-III (MT-10- | |
| | | 021G08aA1. | | | III)>PIR2:S39418 | |
| | | | | | metallothionein 10-III - | |
| | | | | | blue mussel | |
| 301 | AC003016 | Human BAC | 4.30E−07 | SPC57A10_5 | S; pombe chromosome I | 0.00041 |
| | | clone RG134C19 | | | cosmid c57A10; | |
| | | from 8q21, | | | Unknown; | |
| | | complete | | | SPAC57A10; 05; c | |
| | | sequence. | | | unknown, len:606aa, | |
| | | | | | similar to A; nidulans | |
| | | | | | Q00659, sulfur | |
| | | | | | metabolite repression | |
| | | | | | control, (678aa), fasta | |
| | | | | | scores, opt:1355, | |
| 302 | AC003089 | Human BAC | 3.80E−07 | HPBPRECK_1 | Hepatitis B virus type 11 | 0.41 |
| | | clone | | | precore protein (pre−C | |
| | | RG180F08A, | | | region, C) gene, 5′ end | |
| | | complete | | | | |
| | | sequence. | | | | |
| 303 | AC002074 | Human BAC | 2.40E−07 | A47021_1 | Sequence 23 from Patent | 0.0016 |
| | | clone GS056H18 | | | WO9527787; Unnamed | |
| | | from 7q31-q32, | | | protein product; Author- | |
| | | complete | | | given protein sequence is | |
| | | sequence. | | | in conflict with the | |
| | | | | | conceptual | |
| | | | | | translation>GP:A51260_{—} | |
| | | | | | 1 Sequence 23 from | |
| | | | | | Patent WO9614416; | |
| | | | | | Unnamed protein | |
| | | | | | product; Author-given | |
| | | | | | protein sequence is i | |
| 304 | U04980 | Rattus norvegicus | 2.20E−07 | HUMFSHD_1 | Human | 3.30E−08 |
| | | fetal troponin T 3 | | | facioscapulohumeral | |
| | | (fetal TnT3) | | | muscular dystrophy | |
| | | mRNA, partial | | | (FSHD) gene region, | |
| | | cds. | | | D4Z4 tandem repeat unit; | |
| | | | | | ORF | |
| 305 | U68704 | Human | 2.00E−07 | HHV6AGNM | Human herpesvirus-6 | 2.70E−05 |
| | | chromosome | | _96 | (HHV-6) U1102, variant | |
| | | 21q22.3 P1-clone | | | A, complete virion | |
| | | 3804 subclone 4- | | | genome; U88; Cys | |
| | | 52. | | | repeats; this loci is open | |
| | | | | | in all six reading frames, | |
| | | | | | part of IE−A | |
| 306 | U51583 | Rattus norvegicus | 8.70E−08 | AF005370_67 | Alcelaphine herpesvirus | 6.10E−07 |
| | | zinc finger | | | 1 L-DNA, complete | |
| | | homeodomain | | | sequence; Putative | |
| | | enhancer-binding | | | immediate early protein; | |
| | | protein-1 (Zfhep- | | | ORF73; similar to H; | |
| | | 1) mRNA, partial | | | saimiri and KSHV | |
| | | cds. | | | ORF73 | |
| 307 | M80206 | Mus domesticus | 8.10E−08 | I53960 | PRR2 alpha - human | 1.70E−28 |
| | | poliovirus | | | | |
| | | receptor homolog | | | | |
| | | (MPH) mRNA, | | | | |
| | | complete cds. | | | | |
| 308 | M60854 | Human ribosomal | 5.70E−08 | OLVPOL_1 | Caprine arthritis | 0.27 |
| | | protein S16 | | | encephalitis virus (isolate | |
| | | mRNA, complete | | | OVLV-N1) pol protein | |
| | | cds. | | | gene, 3′ end of cds; Nt | |
| | | | | | 2497-2695 from CAEV | |
| | | | | | Co | |
| 309 | U82828 | Homo sapiens | 1.50E−08 | C40201 | artifact-warning | 0.00044 |
| | | ataxia | | | sequence (translated | |
| | | telangiectasia | | | ALU class C) - human | |
| | | (ATM) gene, | | | | |
| | | complete cds. | | | | |
| 310 | Z83836 | Human DNA | 1.40E−08 | HSU64473_1 | Human rheumatoid | 0.34 |
| | | sequence from | | | arthritis synovium | |
| | | PAC 111J24 on | | | immunoglobulin heavy | |
| | | chromosome | | | chain variable region | |
| | | 22q12-qter | | | mRNA, partial | |
| | | contains ESTs. | | | cds>GP:HSU64498_1 | |
| | | | | | Human rheumatoid | |
| | | | | | arthritis synovium | |
| | | | | | immunoglobulin heavy | |
| | | | | | chain variable region | |
| | | | | | mRNA, partial cds | |
| 311 | Z50029 | Caenorhabditis | 1.40E−08 | MMU88984_1 | Mus musculus NIK | 1.70E−50 |
| | | elegans cosmid | | | mRNA, complete cds | |
| | | ZC504, complete | | | | |
| | | sequence. | | | | |
| 312 | AC002351 | Homo sapiens; | 1.20E−08 | D41132 | collagen-related protein 4 | 0.02 |
| | | HTGS phase 1, | | | - Hydra magnipapillata | |
| | | 17 unordered | | | (fragment)>PIR2:S21932 | |
| | | pieces. | | | mini-collagen - Hydra | |
| | | | | | sp.>GP:HSNCOL4_1 | |
| | | | | | Hydra N-COL 4 mRNA | |
| | | | | | for mini-collagen; No | |
| | | | | | start codon | |
| 313 | B65763 | CIT-HSP- | 3.60E−09 | S18106 | type II site−specific | 0.045 |
| | | 2023A12.TR | | | deoxyribonuclease (EC | |
| | | CIT-HSP Homo | | | 3.1.21.4) AbrI - | |
| | | sapiens genomic | | | Azospirillum brasilense | |
| | | clone 2023A 12. | | | | |
| 314 | Z93021 | Human DNA | 2.00E−09 | AB001684_134 | Chlorella vulgaris C-27 | 0.6 |
| | | sequence *** | | | chloroplast DNA, | |
| | | SEQUENCING | | | complete sequence; RNA | |
| | | IN PROGRESS | | | polymerase gamma | |
| | | *** from clone | | | subunit | |
| | | 516C23; HTGS | | | | |
| | | phase 1. | | | | |
| 315 | D88035 | Rat mRNA for | 1.50E−09 | D88035_1 | Rat mRNA for | 1.00E−33 |
| | | glycoprotein | | | glycoprotein specific | |
| | | specific UDP- | | | UDP- | |
| | | glucuronyltransfe | | | glucuronyltransferase, | |
| | | rase, complete | | | complete cds | |
| | | cds. | | | | |
| 316 | U85193 | Human nuclear | 1.30E−10 | VGF1_IBVB | F1 | 1 |
| | | factor I-B2 | | | PROTEIN>PIR1:VF1HB | |
| | | (NF1B2) mRNA, | | | 1 F1 protein - avian | |
| | | complete cds. | | | infectious bronchitis | |
| | | | | | virus (strain | |
| | | | | | Beaudette)>GP:IBACGB | |
| | | | | | _1 Avian infectious | |
| | | | | | bronchitis virus pol | |
| | | | | | protein, spike protein, | |
| | | | | | small virion-associated | |
| | | | | | protein, membrane | |
| | | | | | protein, and nucleocapsid | |
| | | | | | protein gen | |
| 317 | B04719 | cSRL-42G12-u | 7.90E−11 | JC5238 | galactosylceramide−like | 0.31 |
| | | cSRL flow sorted | | | protein, GCP - human | |
| | | Chromosome 11 | | | | |
| | | specific cosmid | | | | |
| | | Homo sapiens | | | | |
| | | genomic clone | | | | |
| | | cSRL-42G12. | | | | |
| 318 | M73506 | Mouse Top-10c (t | 2.80E−11 | A39487 | T-complex protein 10a | 4.10E−16 |
| | | allele) gene. | | | (allele 129) - mouse | |
| 319 | U71148 | Human Xq28 | 1.20E−11 | A56547 | sex-peptide precursor - | 0.4 |
| | | cosmids U225B5 | | | Drosophila suzukii | |
| | | and U236A12, | | | | |
| | | complete | | | | |
| | | sequence. | | | | |
| 320 | Z95116 | Human DNA | 9.90E−13 | ALU2_HUM | !!!! ALU SUBFAMILY | 0.0017 |
| | | sequence *** | | AN | SB WARNING ENTRY | |
| | | SEQUENCING | | | !!!! | |
| | | IN PROGRESS | | | | |
| | | *** from clone | | | | |
| | | 57G9; HTGS | | | | |
| | | phase 1. | | | | |
| 321 | M64795 | Rat MHC class I | 1.70E−14 | STC_DROME | SHUTTLE CRAFT | 1.40E−13 |
| | | antigen gene | | | PROTEIN>GP:DMU093 | |
| | | (RT1-u | | | 06_1 Drosophila | |
| | | haplotype), | | | melanogaster shuttle craft | |
| | | complete cds. | | | protein (stc) mRNA, | |
| | | | | | complete cds; C-terminal | |
| | | | | | 222 amino acids encode a | |
| | | | | | novel single−stranded | |
| | | | | | DNA binding domain | |
| 322 | Y09036 | H. sapiens | 4.20E−15 | AF010403_1 | Homo sapiens ALR | 1 |
| | | NTRK1 gene, | | | mRNA, complete cds; | |
| | | exon 17. | | | Alternatively spliced; | |
| | | | | | similarity to ALL-1 and | |
| | | | | | Drosophila trithorax | |
| 323 | U12523 | Rattus norvegicus | 2.90E−15 | SPBC30D10_4 | S; pombe chromosome II | 2.40E−09 |
| | | ultraviolet B | | | cosmid c30D10; | |
| | | radiation- | | | Hypothetical protein; | |
| | | activated UV98 | | | SPBC30D10; 04, | |
| | | mRNA, partial | | | unknown, len:148aa | |
| | | sequence. | | | | |
| 324 | Z98755 | Human DNA | 2.20E−15 | RPON_HAL | DNA-DIRECTED RNA | 0.019 |
| | | sequence *** | | MA | POLYMERASE | |
| | | SEQUENCING | | | SUBUNIT N (EC | |
| | | IN PROGRESS | | | 2.7.7.6)>PIR2:D41715 | |
| | | *** from clone | | | DNA-directed RNA | |
| | | 76C18; HTGS | | | polymerase II chain | |
| | | phase 1. | | | RPB10 homolog - | |
| | | | | | Haloarcula | |
| | | | | | marismortui>GP:HALH | |
| | | | | | MAENOA_4 | |
| | | | | | H; marismortui tRNA- | |
| | | | | | Leu, HL29, HmaL 13, | |
| | | | | | HmaS9, OrfMMV, | |
| | | | | | OrfMNA, 2- | |
| | | | | | phosphoglycerate dehydr | |
| 325 | M86917 | Human oxysterol- | 1.60E−15 | CEF14H8_2 | Caenorhabditis elegans | 2.10E−18 |
| | | binding protein | | | cosmid F14H8, complete | |
| | | (OSBP) mRNA, | | | sequence; F14H8; 1; | |
| | | complete cds. | | | Similarity to Human | |
| | | | | | oxysterol-binding protein | |
| | | | | | (SW:OXYB_HUMAN) | |
| 326 | AC001231 | Genomic | 1.30E−15 | AC002397_3 | Mouse BAC284H12 | 0.0016 |
| | | sequence from | | | Chromosome 6, complete | |
| | | Human 17, | | | sequence; DRPLA | |
| | | complete | | | | |
| | | sequence. | | | | |
| 327 | AL008626 | Human DNA | 5.30E−16 | TAU48227_1 | Triticum aestivum | 5.90E−05 |
| | | sequence *** | | | soluble starch synthase | |
| | | SEQUENCING | | | mRNA, partial cds | |
| | | IN PROGRESS | | | | |
| | | *** from clone | | | | |
| | | 1114G22; HTGS | | | | |
| | | phase 1. | | | | |
| 328 | L04483 | Human ribosomal | 7.60E−17 | RS21_HUMAN | 40S RIBOSOMAL | 1.40E−09 |
| | | protein S21 | | | PROTEIN | |
| | | (RPS21) mRNA, | | | S21>PIR2:S34108 | |
| | | complete cds. | | | ribosomal protein S21 - | |
| | | | | | human>GP:SSZ84015_1 | |
| | | | | | S; scrofa mRNA; | |
| | | | | | expressed sequence tag | |
| | | | | | (3′; clone c11g10); 40S | |
| | | | | | ribosomal protein S21; | |
| | | | | | Similar to human 40S | |
| | | | | | ribosomal protein | |
| | | | | | S21>GP:HUMRPS21X_{—} | |
| | | | | | 1 Human ribosomal | |
| 329 | AB001899 | Homo sapiens | 6.70E−17 | LRP1_HUMAN | LOW-DENSITY | 1 |
| | | PACE4 gene, | | | LIPOPROTEIN | |
| | | exon 2. | | | RECEPTOR-RELATED | |
| | | | | | PROTEIN 1 | |
| | | | | | PRECURSOR (LRP) | |
| | | | | | (ALPHA-2- | |
| | | | | | MACROGLOBULIN | |
| | | | | | RECEPTOR) (A2MR) | |
| | | | | | (APOLIPOPROTEIN E | |
| | | | | | RECEPTOR) | |
| | | | | | (APOER)>PIR2:S02392 | |
| | | | | | LDL receptor-related | |
| | | | | | protein precursor - | |
| | | | | | human>GP:HSLDLRRL | |
| | | | | | _1 Human mRNA for | |
| | | | | | LDL-recept | |
| 330 | Z98755 | Human DNA | 4.40E−17 | U97553_59 | Murine herpesvirus 68 | 0.06 |
| | | sequence *** | | | strain WUMS, complete | |
| | | SEQUENCING | | | genome; Ribonucleotide | |
| | | IN PROGRESS | | | reductase large | |
| | | *** from clone | | | | |
| | | 76C18; HTGS | | | | |
| | | phase 1. | | | | |
| 331 | AF017187 | Homo sapiens | 3.90E−18 | D84255_1 | Ovophis okinavensis | 0.007 |
| | | LTR HERV-K | | | mitochondrial DNA for | |
| | | repetitive element | | | NADH dehydrogenase | |
| | | fragment | | | subunit 1, partial cds, Ile− | |
| | | ltr_19_9a | | | tRNA, Pro-tRNA, Phe− | |
| | | sequence. | | | tRNA, Gln-tRNA, Met- | |
| | | | | | tRNA and control region | |
| | | | | | (D-loop region); This cds | |
| 332 | B36252 | HS-1038-A2- | 3.10E−18 | PGBM_MOU | BASEMENT | 0.00015 |
| | | G01-MR.abi CIT | | SE | MEMBRANE− | |
| | | Human Genomic | | | SPECIFIC HEPARAN | |
| | | Sperm Library C | | | SULFATE | |
| | | Homo sapiens | | | PROTEOGLYCAN | |
| | | genomic clone | | | CORE PROTEIN | |
| | | Plate = CT 820 | | | PRECURSOR (HSPG) | |
| | | Col = 2 Row = M. | | | (PERLECAN) | |
| | | | | | (PLC)>PIR2:S18252 | |
| | | | | | heparan sulfate | |
| | | | | | proteoglycan - | |
| | | | | | mouse>GP:MUSPERPA | |
| | | | | | _1 Mouse perlecan | |
| | | | | | mRNA, complete cds | |
| 333 | D78255 | Mouse mRNA for | 2.70E−18 | MUSPAP1_1 | Mouse mRNA for PAP- | 3.50E−18 |
| | | PAP-1, complete | | | 1, complete cds | |
| | | cds. | | | | |
| 334 | AC003046 | Human Xp22 | 1.40E−18 | CEC34F6_1 | Caenorhabditis elegans | 0.0015 |
| | | PACs RPC11- | | | cosmid C34F6; C34F6; 1; | |
| | | 263P4 and | | | CDNA EST yk46b12; 5 | |
| | | RPC11-164K3 | | | comes from this gene; | |
| | | complete | | | cDNA EST yk44c4; 5 | |
| | | sequence. | | | comes from this gene; | |
| | | | | | cDNA EST yk46b12; 3 | |
| | | | | | comes from this gene | |
| 335 | AC003002 | Human DNA | 1.40E−18 | MUSZFP0_1 | Mouse mRNA for zinc | 1.30E−19 |
| | | from overlapping | | | finger protein, partial | |
| | | chromosome 19- | | | sequence | |
| | | specific cosmids | | | | |
| | | R29515 and | | | | |
| | | R28253, genomic | | | | |
| | | sequence, | | | | |
| | | complete | | | | |
| | | sequence. | | | | |
| 336 | Y15054 | Rattus norvegicus | 3.40E−19 | HS4U2IR2_1 | Epstein-Barr virus | 2.00E−06 |
| | | mRNA for 70 | | | (AG876 isolate) U2-IR2 | |
| | | kDa tumor | | | domain encoding nuclear | |
| | | specific antigen, | | | protein EBNA2, | |
| | | partial. | | | complete cds; Nuclear | |
| | | | | | antigen 2 | |
| 337 | Z97876 | Human DNA | 1.30E−19 | AF003535_1 | Homo sapiens L1 | 7.00E−05 |
| | | sequence *** | | | element ORF2-like | |
| | | SEQUENCING | | | protein gene, partial cds | |
| | | IN PROGRESS | | | | |
| | | *** from clone | | | | |
| | | 295C6; HTGS | | | | |
| | | phase 1. | | | | |
| 338 | M97159 | Mouse (clone | 1.10E−19 | A26882 | pIL2 hypothetical protein | 0.2 |
| | | pIL2) B1 | | | - rat | |
| | | dispersed repeat | | | (fragment)>GP:RATTD | |
| | | unit. | | | R_1 Rat growth and | |
| | | | | | transformation-dependent | |
| | | | | | mRNA, 3′ end; Growth | |
| | | | | | and transformation | |
| | | | | | dependent protein | |
| 339 | U30817 | Bos taurus very- | 4.70E−20 | ACDV_RAT | ACYL-COA | 8.10E−25 |
| | | long-chain acyl- | | | DEHYDROGENASE, | |
| | | CoA | | | VERY-LONG-CHAIN | |
| | | dehydrogenase | | | SPECIFIC | |
| | | mRNA, nuclear | | | PRECURSOR (EC | |
| | | gene encoding | | | 1.3.99.-) | |
| | | mitochondrial | | | (VLCAD)>PIR2:A54872 | |
| | | protein, complete | | | acyl-CoA dehydrogenase | |
| | | cds. | | | (EC 1.3.99.-) very-long- | |
| | | | | | chain-specific precursor - | |
| | | | | | rat>GP:RATVLCAD_1 | |
| | | | | | Rat mRNA for very- | |
| | | | | | long-chain Acyl-CoA | |
| | | | | | dehydrogenase, compl | |
| 340 | Y11535 | H. sapiens mRNA | 2.80E−20 | ALU1_HUM | !!!! ALU SUBFAMILY J | 0.00027 |
| | | for SHOXb | | AN | WARNING ENTRY !!!! | |
| | | protein. | | | | |
| 341 | AL008730 | Human DNA | 7.10E−21 | C40201 | artifact-warning | 0.001 |
| | | sequence *** | | | sequence (translated | |
| | | SEQUENCING | | | ALU class C)- human | |
| | | IN PROGRESS | | | | |
| | | *** from clone | | | | |
| | | 487J7; HTGS | | | | |
| | | phase 1. | | | | |
| 342 | U96629 | Human | 5.30E−23 | ALU1_HUM | !!!! ALU SUBFAMILY J | 3.80E−10 |
| | | chromosome 8 | | AN | WARNING ENTRY !!!! | |
| | | BAC clone | | | | |
| | | CIT987SK-2A8 | | | | |
| | | complete | | | | |
| | | sequence. | | | | |
| 343 | U95743 | Homo sapiens | 2.10E−24 | UROM_HUM | UROMODULIN | 1 |
| | | chromosome 16 | | AN | PRECURSOR (TAMM- | |
| | | BAC clone | | | HORSFALL URINARY | |
| | | CIT987-SK65D3, | | | GLYCOPROTEIN) | |
| | | complete | | | (THP)>PIR2:A30452 | |
| | | sequence. | | | uromodulin precursor- | |
| | | | | | human>GP:HUMUMOD | |
| | | | | | _1 Human uromodulin | |
| | | | | | (Tamm-Horsfall | |
| | | | | | glycoprotein) mRNA, | |
| | | | | | complete cds; | |
| | | | | | Uromodulin precursor | |
| 344 | U15972 | Mus musculus | 4.00E−25 | S20790 | extensin- | 0.34 |
| | | homeobox | | | almond>GP:PAEXTS_1 | |
| | | (Hoxa7) gene, | | | P; amygdalus mRNA for | |
| | | complete cds. | | | extensin | |
| 345 | U15972 | Mus musculus | 4.00E−25 | CA24_CAEE | COLLAGEN ALPHA | 0.1 |
| | | homeobox | | L | 2(IV) CHAIN | |
| | | (Hoxa7) gene, | | | PRECURSOR>GP:CEC | |
| | | complete cds. | | | OLA2IV_2 C; elegans | |
| | | | | | a2(IV) collagen gene; | |
| | | | | | Alternatively spliced | |
| | | | | | transcript | |
| 346 | Z66242 | H. sapiens CpG | 4.80E−26 | CEC35A5_8 | Caenorhabditis elegans | 7.70E−19 |
| | | island DNA | | | cosmid C35A5, complete | |
| | | genomic Mse1 | | | sequence; C35A5; 8; | |
| | | fragment, clone | | | CDNA EST yk31f6; 5 | |
| | | 84a4, reverse read | | | comes from this gene; | |
| | | cpg84a4.rt1a. | | | cDNA EST yk38h1; 3 | |
| | | | | | comes from this gene; | |
| | | | | | cDNA EST yk38h1; 5 | |
| | | | | | comes from this gene; | |
| 347 | L25331 | Rattus norvegicus | 3.90E−26 | LYSH_CHICK | PROCOLLAGEN- | 1.10E−43 |
| | | lysyl hydroxylase | | | LYSINE,2- | |
| | | mRNA, complete | | | OXOGLUTARATE 5- | |
| | | cds. | | | DIOXYGENASE | |
| | | | | | PRECURSOR (EC | |
| | | | | | 1.14.11.4) (LYSYL | |
| | | | | | HYDROXYLASE)>PIR | |
| | | | | | 2:A23742 procollagen- | |
| | | | | | lysine 5-dioxygenase (EC | |
| | | | | | 1.14.11.4) precursor- | |
| | | | | | chicken>GP:CHKLYH_{—} | |
| | | | | | 1 Chicken lysyl | |
| | | | | | hydroxylase mRNA, | |
| | | | | | complete cds | |
| 348 | L81569 | Drosophila | 3.30E−26 | CELC52B9_2 | Caenorhabditis elegans | 8.40E−29 |
| | | melanogaster | | | cosmid C52B9; Coded | |
| | | (subclone 2_d7 | | | for by C; elegans cDNA | |
| | | from P1 DS04260 | | | cm11d6; weakly similar | |
| | | (D68)) DNA | | | to S; cervisiae PTM1 | |
| | | sequence, | | | precursor (SP:P32857) | |
| | | complete | | | | |
| | | sequence. | | | | |
| 349 | U78082 | Human RNA | 2.30E−26 | HSU78082_1 | Human RNA polymerase | l.50E−16 |
| | | polymerase | | | transcriptional regulation | |
| | | transcriptional | | | mediator (h- MED6) | |
| | | regulation | | | mRNA, complete cds; H- | |
| | | mediator (h- | | | Med6p | |
| | | MED6) mRNA, | | | | |
| | | complete cds. | | | | |
| 350 | U43381 | Human Down | 2.10E−28 | HSMRNAEB_1 | H; sapiens genomic DNA, | 0.18 |
| | | Syndrome region | | | integration site for | |
| | | of chromosome | | | Epstein-Barr virus; | |
| | | 21 DNA. | | | Hypothetical protein | |
| 351 | D50416 | Mouse mRNA for | 2.50E−29 | A29947 | prostaglandin- | 0.81 |
| | | AREC3, | | | endoperoxide synthase | |
| | | complete cds. | | | (EC 1.14.99.1) precursor- | |
| | | | | | sheep>GP:SHPCOXA_1 | |
| | | | | | Sheep prostaglandin | |
| | | | | | endoperoxide synthetase | |
| | | | | | (cyclooxygenase), | |
| | | | | | complete cds; | |
| | | | | | Cyclooxygenase | |
| | | | | | precursor (EC 1; 14; 99; 1) | |
| 352 | U85193 | Human nuclear | 2.20E−29 | CFU30222_1 | Crithidia fasciculata fully | 0.53 |
| | | factor I-B2 | | | edited ATPase subunit 6 | |
| | | (NFIB2) mRNA, | | | (MURF4) mRNA, partial | |
| | | complete cds. | | | cds; Cryptogene | |
| 353 | Z92826 | Caenorhabditis | 1.10E−30 | SPAC1B3_5 | S; pombe chromosome I | 3.20E−35 |
| | | elegans DNA *** | | | cosmid c1B3; | |
| | | SEQUENCING | | | Hypothetical protein; | |
| | | IN PROGRESS | | | SPAC1B3; 05, probable | |
| | | *** from clone | | | transcriptional regulator, | |
| | | C18D11; HTGS | | | len:630aa, similar eg; to | |
| | | phase 1. | | | YIL038C, | |
| | | | | | NOT3_YEAST, P06102, | |
| | | | | | general negative | |
| | | | | | regulator, | |
| 354 | L09604 | Homo sapiens | 3.70E−32 | PVU72769_1 | Phaseolus vulgaris | 0.00049 |
| | | differentiation- | | | PvPRP-12 (Pvprp1-12) | |
| | | dependent A4 | | | mRNA, partial cds; | |
| | | protein mRNA, | | | Similar to cell wall | |
| | | complete cds. | | | proline rich | |
| | | | | | protein>GP:PVU72769_{—} | |
| | | | | | 1 Phaseolus vulgaris | |
| | | | | | PvPRP-12 (Pvprp1-12) | |
| | | | | | mRNA, partial cds; | |
| | | | | | Similar to cell wall | |
| | | | | | proline rich protein | |
| 355 | B42455 | HS-1055-B2- | 1.30E−32 | CELT05H4_8 | Caenorhabditis elegans | 6.90E−14 |
| | | G03-MR.abi CIT | | | cosmid T05H4; Similar | |
| | | Human Genomic | | | to the beta transducin | |
| | | Sperm Library C | | | family; coded for by C; | |
| | | Homo sapiens | | | elegans cDNA | |
| | | genomic clone | | | yk156e11; 3; coded for by | |
| | | Plate'2 CT 777 | | | C; elegans cDNA | |
| | | Col'2 6 Row'2 N. | | | yk14c8; 3; coded for by | |
| | | | | | C; elegans cDNA | |
| 356 | AF001905 | Homo sapiens | 1.80E−33 | I38344 | titin - human | 1 |
| | | cosmids E079, | | | | |
| | | B0920 and A8 | | | | |
| | | from Xq25 X- | | | | |
| | | linked | | | | |
| | | lymphoproliferative | | | | |
| | | disease gene | | | | |
| | | candidate region, | | | | |
| | | complete | | | | |
| | | sequence. | | | | |
| 357 | E03743 | DNA sequence | 1.10E−34 | CELC03A7_2 | Caenorhabditis elegans | 0.59 |
| | | including male | | | cosmid C03A7; Weak | |
| | | hormone | | | similarity to serotonin | |
| | | dependent gene | | | receptors | |
| | | derived from | | | | |
| | | hamster | | | | |
| | | frankorgan. | | | | |
| 358 | U31199 | Human laminin | 1.20E−35 | B44018 | laminin B2t chain - | 1.20E−14 |
| | | gamma2 chain | | | human>GP:HSLAMB2T | |
| | | gene (LAMC2), | | | B_1 H; sapiens mRNA | |
| | | exon 22 and | | | for laminin | |
| | | flanking | | | | |
| | | sequences. | | | | |
| 359 | D14678 | Human mRNA | 2.00E−36 | D49544_1 | Mouse mRNA for | 1.20E−23 |
| | | for kinesin- | | | KIFC1, complete cds | |
| | | related protein, | | | | |
| | | partial cds. | | | | |
| 360 | AB000425 | Porcine DNA for | 8.20E−38 | POL4_DROME | RETROVIRUS- | 0.65 |
| | | endopeptidase | | | RELATED POL | |
| | | 24.16, exon 16 | | | POLYPROTEIN | |
| | | and complete cds. | | | (PROTEASE (EC | |
| | | | | | 3.4.23.-); REVERSE | |
| | | | | | TRANSCRIPTASE (EC | |
| | | | | | 2.7.7.49); | |
| | | | | | ENDONUCLEASE) | |
| | | | | | (TRANSPOSON | |
| | | | | | 412)>PIR1:GNFF42 | |
| | | | | | retrovirus-related pol | |
| | | | | | polyprotein - fruit fly | |
| | | | | | (Drosophila | |
| | | | | | melanogaster) transposon | |
| | | | | | 412>GP:DMRT412G_4 | |
| 361 | U39875 | Rattus norvegicus | 8.80E−42 | I56333 | apolipoprotein B - rat | 0.23 |
| | | EF-hand Ca2+- | | | (fragment)>GP:RATAP | |
| | | binding protein | | | OLPB_1 Rattus | |
| | | p22 mRNA, | | | norvegicus (clone rb9E) | |
| | | complete cds. | | | apolipoprotein B apoB | |
| | | | | | mRNA, 3′ end | |
| 362 | L09647 | Rattus norvegicus | 6.60E−42 | HN3B_RAT | HEPATOCYTE | 8.10E−25 |
| | | hepatocyte | | | NUCLEAR FACTOR 3- | |
| | | nuclear factor 3a | | | BETA (HNF- | |
| | | (HNF-3 beta) | | | 3B)>GP:RATHNF3B_1 | |
| | | mRNA, complete | | | Rattus norvegicus | |
| | | cds. | | | hepatocyte nuclear factor | |
| | | | | | 3a (HNF-3 beta) mRNA, | |
| | | | | | complete | |
| | | | | | cds>TFD:TFDP01611 - | |
| | | | | | Polypeptides entry for | |
| | | | | | factor HNF-3 (beta) | |
| 363 | D25538 | Human mRNA | 4.10E−43 | CELC34D4_12 | Caenorhabditis elegans | 0.018 |
| | | for KIAA0037 | | | cosmid C34D4 | |
| | | gene, complete | | | | |
| | | cds. | | | | |
| 364 | Z56764 | H. sapiens CpG | 1.40E−43 | S75263 | hypothetical protein- | 0.0028 |
| | | island DNA | | | Synechocystis sp. (PCC | |
| | | genomic Mse1 | | | 6803)>GP:D90904_29 | |
| | | fragment, clone | | | Synechocystis sp; | |
| | | 13f7, reverse read | | | PCC6803 complete | |
| | | cpg13f7.rt1a. | | | genome, 6/27, 630555- | |
| | | | | | 781448; Hypothetical | |
| | | | | | protein; ORF_ID:sll0983 | |
| 365 | AC002636 | *** | 8.40E−44 | DMU95760_1 | Drosophila melanogaster | 3.40E−51 |
| | | SEQUENCING | | | strawberry notch (sno) | |
| | | IN PROGRESS | | | mRNA, complete cds; | |
| | | *** Drosophila | | | Notch pathway | |
| | | melanogaster | | | component; nuclear | |
| | | (subclone 2_g4 | | | protein | |
| | | from P1 DS03323 | | | | |
| | | (D127)) DNA | | | | |
| | | sequence; HTGS | | | | |
| | | phase 2. | | | | |
| 366 | J05499 | Rattus norvegicus | 8.00E−44 | GLSL_RAT | GLUTAMINASE, | 8.00E−29 |
| | | L-glutamine | | | LIVER ISOFORM | |
| | | amidohydrolase | | | PRECURSOR (EC | |
| | | mRNA, complete | | | 3.5.1.2) | |
| | | cds. | | | (GLS)>GP:RATGAH_1 | |
| | | | | | Rattus norvegicus L- | |
| | | | | | glutamine | |
| | | | | | amidohydrolase mRNA, | |
| | | | | | complete cds | |
| 367 | U95760 | Drosophila | 5.00E−45 | DMU95760_1 | Drosophila melanogaster | 4.80E−45 |
| | | melanogaster | | | strawberry notch (sno) | |
| | | strawberry notch | | | mRNA, complete cds; | |
| | | (sno) mRNA, | | | Notch pathway | |
| | | complete cds. | | | component; nuclear | |
| | | | | | protein | |
| 368 | L10106 | Mus musculus | 4.10E−45 | PTPK_HUMAN | PROTEIN-TYROSINE | 4.70E−16 |
| | | protein tyrosine | | | PHOSPHATASE | |
| | | phosphate | | | KAPPA PRECURSOR | |
| | | mRNA, complete | | | (EC 3.1.3.48) (R-PTP- | |
| | | cds. | | | KAPPA)>GP:HSPTPKA | |
| | | | | | P_1 H; sapiens mRNA for | |
| | | | | | phosphotyrosine | |
| | | | | | phosphatase kappa; | |
| | | | | | Human phosphotyrosine | |
| | | | | | phosphatase kappa | |
| 369 | D17218 | Human HepG2 3′ | 9.40E−47 | MMU53563_1 | Mus musculus Brg1 | 0.00012 |
| | | region MboI | | | mRNA, partial cds; N- | |
| | | cDNA, clone | | | terminal region of the | |
| | | hmd3g02m3. | | | protein | |
| 370 | U78310 | Homo sapiens | 8.10E−48 | HSU78310_1 | Homo sapiens pescadillo | 1.10E−21 |
| | | pescadillo | | | mRNA, complete cds | |
| | | mRNA, complete | | | | |
| | | cds. | | | | |
| 371 | AC000399 | Genomic | 7.40E−48 | KIP2_YEAST | KINESIN-LIKE | 0.14 |
| | | sequence from | | | PROTEIN | |
| | | Mouse 9, | | | KIP2>PIR1:C42640 | |
| | | complete | | | kinesin-related protein | |
| | | sequence. | | | KIP2- yeast | |
| | | | | | (Saccharomyces | |
| | | | | | cerevisiae)>GP:SCKIP2 | |
| | | | | | XVI_2 S; cerevisiae PEP4 | |
| | | | | | and KIP2 genes encoding | |
| | | | | | PEP4 proteinase (partial) | |
| | | | | | and kinesin-related | |
| | | | | | protein | |
| | | | | | KIP2>GP:SCLACHXVI | |
| | | | | | _17 S; cerev | |
| 372 | AC002327 | *** | 1.40E−48 | CHKC1A205_1 | Chicken alpha-2 type−1 | 0.024 |
| | | SEQUENCING | | | collagen; amino acids- 16 | |
| | | IN PROGRESS | | | to 3; Precollagen alpha-2 | |
| | | *** Genomic | | | | |
| | | sequence from | | | | |
| | | Mouse 7; HTGS | | | | |
| | | phase 1, 3 | | | | |
| | | unordered pieces. | | | | |
| 373 | X67016 | H. sapiens mRNA | 9.00E−49 | CED2085_2 | Caenorhabditis elegans | 0.14 |
| | | for amphiglycan. | | | cosmid D2085, complete | |
| | | | | | sequence; D2085; 1; | |
| | | | | | Similar to glutamine− | |
| | | | | | dependent carbamoyl- | |
| | | | | | phosphate synthase, | |
| | | | | | aspartate | |
| | | | | | carbamoyltransferase, | |
| | | | | | dihydroorotase; cDNA | |
| | | | | | EST | |
| | | | | | cm16f3>GP:CED2085_2 | |
| | | | | | Caenorhabditis elegans | |
| | | | | | cosmid D2085; D | |
| 374 | L10409 | Mouse fork head | 1.50E−49 | MMU04197_1 | Mus musculus HNF3 | 1.20E−30 |
| | | related protein | | | beta transcription factor | |
| | | (HNF-3beta) | | | (HNF3b) mRNA, partial | |
| | | mRNA, complete | | | cds; Sequence of this | |
| | | cds. | | | partial cDNA begins in | |
| | | | | | the first third of the | |
| | | | | | conserved | |
| | | | | | HNF3/forkhead DNA | |
| | | | | | binding domain | |
| 375 | U01139 | Mus musculus | 1.20E−49 | SPBC3D5_14 | S; pombe chromosome II | 0.00091 |
| | | B6D2F1 clone | | | cosmid c3D5; Unknown; | |
| | | 2C11B mRNA. | | | SPBC3D5; 14c, | |
| | | | | | unknown; partial; serine | |
| | | | | | rich, len:309aa, similar | |
| | | | | | eg; to YNL283C, | |
| | | | | | YN23_YEAST, P53832, | |
| | | | | | hypothetical 52; 3 kd | |
| | | | | | protein, (503aa), | |
| 376 | Z82170 | Human DNA | 9.00E−50 | BSU55043_3 | Bacillus subtilis plasmid | 0.025 |
| | | sequence from | | | pPOD2000 Rep, RapAB, | |
| | | PAC 326L13 | | | RapA, ParA, ParB, and | |
| | | containing brain- | | | ParC genes, complete | |
| | | 4 mRNA ESTs | | | cds; ORF3 | |
| | | and polymorphic | | | | |
| | | CA repeat. | | | | |
| 377 | Z99289 | Human DNA | 7.70E−50 | A64431 | hypothetical protein | 5.60E−05 |
| | | sequence *** | | | MJ1050- | |
| | | SEQUENCING | | | Methanococcus | |
| | | IN PROGRESS | | | jannaschii>GP:MJU6754 | |
| | | *** from clone | | | 8_2 Methanococcus | |
| | | 142L7; HTGS | | | jannaschii from bases | |
| | | phase 1. | | | 986219 to 996377 | |
| | | | | | (section 90 of 150) of the | |
| | | | | | complete genome; M; | |
| | | | | | jannaschii predicted | |
| | | | | | coding region MJ1050; | |
| | | | | | Identified by GeneMark; | |
| | | | | | putativ | |
| 378 | X98260 | H. sapiens mRNA | 6.20E−50 | ZRF1_MOUSE | ZUOTIN RELATED | 3.90E−30 |
| | | for M-phase | | | FACTOR>GP:MMU532 | |
| | | phosphoprotein, | | | 08_1 Mus musculus | |
| | | mpp11. | | | zuotin related factor | |
| | | | | | (ZRF1) mRNA, complete | |
| | | | | | cds; Similar to DnaJ | |
| | | | | | encoded by GenBank | |
| | | | | | Accession Number | |
| | | | | | L16953 | |
| 379 | M18981 | Human prolactin | 9.00E−52 | S106_HUMAN | CALCYCLIN | 8.80E−24 |
| | | receptor- | | | (PROLACTIN | |
| | | associated protein | | | RECEPTOR | |
| | | (PRA) gene, | | | ASSOCIATED | |
| | | complete cds. | | | PROTEIN) (PRA) | |
| | | | | | (GROWTH FACTOR- | |
| | | | | | INDUCIBLE PROTEIN | |
| | | | | | 2A9) (S100 CALCIUM- | |
| | | | | | BINDING PROTEIN | |
| | | | | | A6)>PIR1:BCHUY | |
| | | | | | calcyclin- | |
| | | | | | human>GP:HUMCACY | |
| | | | | | _1 Human calcyclin | |
| | | | | | gene, complete | |
| | | | | | cds>GP:HUMCACYA_1 | |
| | | | | | Human prolactin recept | |
| 380 | AB006622 | Homo sapiens | 1.60E−53 | S33015 | hypothetical protein- | 0.00088 |
| | | mRNA for | | | human herpesvirus 4 | |
| | | KIAA0284 gene, | | | | |
| | | partial cds. | | | | |
| 381 | U53225 | Human sorting | 1.80E−55 | G02522 | sorting nexin 1- | 9.20E−50 |
| | | nexin 1 (SNX1) | | | human>GP:HSU53225_1 | |
| | | mRNA, complete | | | Human sorting nexin 1 | |
| | | cds. | | | (SNX1) mRNA, | |
| | | | | | complete cds | |
| 382 | Z92844 | Human DNA | 6.50E−56 | D14487_1 | Lentinus edodes | 1 |
| | | sequence from | | | Le; MFB1 mRNA, | |
| | | PAC 435C23 on | | | complete cds | |
| | | chromosome X. | | | | |
| | | Contains ESTs. | | | | |
| 383 | D87450 | Human mRNA | 4.30E−56 | D87450_1 | Human mRNA for | 4.30E−30 |
| | | for KIAA0261 | | | KIAA0261 gene, partial | |
| | | gene, partial cds. | | | cds; Similar to | |
| | | | | | D; melanogaster parallel | |
| | | | | | sister chromatids protein | |
| 384 | AC002301 | *** | 9.80E−57 | S62328 | kinesin-like DNA | 2.60E−27 |
| | | SEQUENCING | | | binding protein KID- | |
| | | IN PROGRESS | | | human>GP:HUMKID_1 | |
| | | *** Human | | | Human mRNA for Kid | |
| | | chromosome + | | | (kinesin-like DNA | |
| | | 16p11.2 BAC | | | binding protein), | |
| | | clone CIT987SK- | | | complete cds | |
| | | A-328A3; HTGS | | | | |
| | | phase 2, 1 | | | | |
| | | ordered pieces. | | | | |
| 385 | L29766 | Homo sapiens | 7.30E−57 | HSBCTCF4_1 | Homo sapiens mRNA for | 2.30E−05 |
| | | epoxide hydrolase | | | hTCF-4 | |
| | | (EPHX) gene, | | | | |
| | | complete cds. | | | | |
| 386 | U58884 | Mus musculus | 3.30E−58 | MMU58884_1 | Mus musculus SH3- | 6.00E−43 |
| | | SH3-containing | | | containing protein | |
| | | protein SH3P7 | | | SH3P7 mRNA, complete | |
| | | mRNA, complete | | | cds; similar to Human | |
| | | cds. similar to | | | Drebrin; SH3-containing | |
| | | Human Drebrin. | | | protein; similar to human | |
| | | | | | drebrin | |
| 387 | Y15054 | Rattus norvegicus | 9.50E−59 | RNY15054_1 | Rattus norvegicus mRNA | 4.70E−45 |
| | | mRNA for 70 | | | for 70 kDa tumor specific | |
| | | kDa tumor | | | antigen, partial; 70 kD | |
| | | specific antigen, | | | tumor-specific antigen | |
| | | partial. | | | | |
| 388 | AC000406 | *** | 7.40E−59 | <NONE> | <NONE> | <NONE> |
| | | SEQUENCING | | | | |
| | | IN PROGRESS | | | | |
| | | *** Human | | | | |
| | | Chromosome 11 | | | | |
| | | overlapping pacs | | | | |
| | | pDJ235k10 and | | | | |
| | | pDJ239b22; | | | | |
| | | HTGS phase 1, | | | | |
| | | 17 unordered | | | | |
| | | pieces. | | | | |
| 389 | L42612 | Homo sapiens | 3.60E−59 | KRHUEA | keratin, type II | 7.60E−30 |
| | | keratin 6 isoform | | | cytoskeletal - human | |
| | | K6f (KRT6F) | | | (fragment)>GP:HSKER | |
| | | mRNA, complete | | | A_1 Human messenger | |
| | | cds. | | | fragment encoding | |
| | | | | | cytoskeletal keratin (type | |
| | | | | | II); mRNA from cultured | |
| | | | | | epidermal cells from | |
| | | | | | human | |
| | | | | | foreskin>GP:HUMKER5 | |
| | | | | | 6K_1 Human 56k | |
| | | | | | cytoskeletal type II | |
| | | | | | keratin mRNA | |
| 390 | L29766 | Homo sapiens | 2.70E−60 | EGR2_HUMAN | EARLY GROWTH | 7.80E−06 |
| | | epoxide hydrolase | | | RESPONSE PROTEIN 2 | |
| | | (EPHX) gene, | | | (EGR-2) (KROX-20 | |
| | | complete cds. | | | PROTEIN) | |
| | | | | | (AT591)>GP:HUMEGR | |
| | | | | | 2A_1 Human early | |
| | | | | | growth response 2 | |
| | | | | | protein (EGR2) mRNA, | |
| | | | | | complete | |
| | | | | | cds>TFD:TFDP00485 - | |
| | | | | | Polypeptides entry for | |
| | | | | | factor Egr-2 | |
| 391 | L08758 | Mus musculus | 1.40E−60 | PAALGYGE | P; aeruginosa algY gene; | 0.00031 |
| | | homeobox protein | | N_1 | Alginate lyase | |
| | | (Hox A 10) gene, | | | | |
| | | 5′ end of cds. | | | | |
| 392 | I29058 | Sequence 3 from | 4.20E−61 | JC5106 | stromal cell-derived | 1.50E−32 |
| | | patent US | | | factor 2- | |
| | | 5576423. | | | human>GP:D50645_1 | |
| | | | | | Human mRNA for SDF2, | |
| | | | | | complete cds; Stroma | |
| | | | | | cell-derived factor-2 | |
| 393 | I29058 | Sequence 3 from | 4.20E−61 | JC5106 | stromal cell-derived | 1.50E−32 |
| | | patent US | | | factor 2 - | |
| | | 5576423. | | | human>GP:D50645_1 | |
| | | | | | Human mRNA for SDF2, | |
| | | | | | complete cds; Stroma | |
| | | | | | cell-derived factor-2 | |
| 394 | U46067 | Capra hircus | 1.90E−62 | CHU46067_1 | Capra hircus beta- | 2.70E−39 |
| | | beta-mannosidase | | | mannosidase mRNA, | |
| | | mRNA, complete | | | complete cds | |
| | | cds. | | | | |
| 395 | U40747 | Mus musculus | 6.90E−63 | S64713 | formin binding protein | 3.00E−46 |
| | | formin binding | | | 11 - mouse | |
| | | protein 11 | | | (fragment)>GP:MMU40 | |
| | | mRNA, partial | | | 747_1 Mus musculus | |
| | | cds. | | | formin binding protein | |
| | | | | | 11 mRNA, partial cds; | |
| | | | | | FBP 11; Formin binding | |
| | | | | | protein 11; tandem | |
| | | | | | WWP/WW domains | |
| | | | | | separated by 15 amino | |
| | | | | | acid linker | |
| 396 | M36164 | Human | 1.10E−63 | BHT1UL_12 | Bovine herpesvirus type | 0.003 |
| | | glyceraldehyde−3- | | | 1 UL22-35 genes; | |
| | | phosphate | | | UL26; 5>GP:BHU31809_{—} | |
| | | dehydrogenase | | | 2 Bovine herpesvirus 1 | |
| | | mRNA, 3′ flank. | | | maturational proteinase | |
| | | | | | (UL26) gene, complete | |
| | | | | | cds, and scaffold protein | |
| | | | | | (UL26; 5) gene, complete | |
| | | | | | cds | |
| 397 | Y09036 | H. sapiens | 7.30E−65 | MMU39060_1 | Mus musculus | 0.0054 |
| | | NTRK1 gene, | | | glucocorticoid receptor | |
| | | exon 17. | | | interacting protein 1 | |
| | | | | | (GRIP1) mRNA, | |
| | | | | | complete cds; Hormone− | |
| | | | | | dependent interaction | |
| | | | | | with hormone binding | |
| | | | | | domains of steroid | |
| | | | | | receptors; transactivation | |
| 398 | U17901 | Rattus norvegicus | 2.70E−70 | JC4239 | phospholipase A2- | 8.40E−17 |
| | | phospholipase A- | | | activating protein - rat | |
| | | 2-activating | | | | |
| | | protein (plap) | | | | |
| | | mRNA, complete | | | | |
| | | cds. | | | | |
| 399 | D12646 | Mouse kif4 | 1.70E−74 | KIF4_MOUSE | KINESIN-LIKE | 1.10E−44 |
| | | mRNA for | | | PROTEIN | |
| | | microtubule− | | | KIF4>PIR2:A54803 | |
| | | based motor | | | microtubule−associated | |
| | | protein KIF4, | | | motor KIF4 - | |
| | | complete cds. | | | mouse>GP:MUSKIF4_1 | |
| | | | | | Mouse kif4 mRNA for | |
| | | | | | microtubule−based motor | |
| | | | | | protein KIF4, complete | |
| | | | | | cds; ATP-binding site: | |
| | | | | | base980- 1037, motor | |
| | | | | | domain: base732- 1781, | |
| | | | | | alpha-helical co | |
| 400 | AF007860 | Xenopus laevis | 4.60E−75 | AF007862_1 | Mus musculus mm-Mago | 6.50E−68 |
| | | xl-Mago mRNA, | | | mRNA, complete cds; | |
| | | complete cds. | | | Similar to Drosophila | |
| | | | | | melanogaster Mago | |
| | | | | | protein | |
| 401 | I45565 | Sequence 15 from | 2.30E−82 | RNU57391_1 | Rattus norvegicus FceRI | 9.90E−42 |
| | | patent US | | | gamma-chain interacting | |
| | | 5637463. | | | protein SH2-B (SH2-B) | |
| | | | | | mRNA, complete cds; | |
| | | | | | Putative FceRI gamma | |
| | | | | | ITAM interacting | |
| | | | | | protein; SH2 domain- | |
| | | | | | containing protein B; | |
| | | | | | Method: conceptual | |
| 402 | U29156 | Mus musculus | 1.00E−85 | MMU29156_1 | Mus musculus eps15R | 4.90E−62 |
| | | eps15R mRNA, | | | mRNA, complete cds; | |
| | | complete cds. | | | Involved in signaling by | |
| | | | | | the epidermal growth | |
| | | | | | factor receptor; Method: | |
| | | | | | conceptual translation | |
| | | | | | supplied by author | |
| 403 | U70139 | Mus musculus | 1.00E−85 | MMU70139_1 | Mus musculus putative | 7.20E−66 |
| | | putative CCR4 | | | CCR4 protein mRNA, | |
| | | protein mRNA, | | | partial cds; Similar to | |
| | | partial cds. | | | yeast transcription factor | |
| | | | | | CCR4; transcriptional | |
| | | | | | readthrough occurs with | |
| | | | | | transcription being | |
| | | | | | initiated at the IAP and | |
| | | | | | continues | |
| 404 | U82626 | Rattus norvegicus | 7.60E−96 | RNU82626_1 | Rattus norvegicus | 8.20E−58 |
| | | basement | | | basement membrane− | |
| | | membrane− | | | associated chondroitin | |
| | | associated | | | proteoglycan Bamacan | |
| | | chondroitin | | | mRNA, complete cds; | |
| | | proteoglycan | | | Chondroitin sulfate | |
| | | Bamacan mRNA, | | | proteoglycan; CSPG | |
| | | complete cds. | | | | |
| 405 | L09604 | Homo sapiens | 2.00E−35 | <NONE> | <NONE> | <NONE> |
| | | differentiation- | | | | |
| | | dependent A4 | | | | |
| | | protein mRNA, | | | | |
| | | complete cds. | | | | |
| 406 | AB000516 | Homo sapiens | 0.41 | POLG_TUMVQ | GENOME | 2.9 |
| | | mRNA for DSIF | | | POLYPROTEIN | |
| | | p160, complete | | | (CONTAINS: N- | |
| | | cds | | | TERMINAL | |
| | | | | | PROTEIN; HELPER | |
| | | | | | COMPONENT | |
| | | | | | PROTEINASE (EC | |
| | | | | | 3.4.22.-) (HC-PRO); | |
| | | | | | 42-50 KD PROTEIN; | |
| | | | | | CYTOPLASMIC | |
| | | | | | INCLUSION | |
| | | | | | PROTEIN (CI); 6 KD | |
| | | | | | PROTEIN; VPG | |
| | | | | | PROTEIN; | |
| | | | | | NUCLEAR | |
| | | | | | INCLUSION | |
| | | | | | PROTEIN A (NI-A) | |
| 407 | Z94753 | Human DNA | 0.004 | <NONE> | <NONE> | <NONE> |
| | | sequence from | | | | |
| | | PAC 465G10 on | | | | |
| | | chromosome X | | | | |
| | | contains Menkes | | | | |
| | | Disease (ATP7A) | | | | |
| | | putative Cu++- | | | | |
| | | transporting P- | | | | |
| | | type ATPase | | | | |
| | | exons 22, 23 and | | | | |
| | | STS | | | | |
| 408 | AB011123 | Homo sapiens | 0 | MI15_CAEEL | Q23356 | 2.00E−51 |
| | | mRNA for | | | Caenorhabditis | |
| | | KIAA0551 | | | elegans . | |
| | | protein, partial | | | serine/threonine− | |
| | | cds | | | protein kinase mig-15 | |
| | | | | | (ec 2.7.1.-). 11/98 | |
| 409 | D17218 | Human HepG2 3′ | e−123 | NARG_BACSU | NITRATE | 9.9 |
| | | region MboI | | | REDUCTASE | |
| | | cDNA, clone | | | ALPHA CHAIN (EC | |
| | | hmd3g02m3 | | | 1.7.99.4) | |
| 410 | M95098 | Bos taurus | 1.1 | HAIR_MOUSE | HAIRLESS | 8.00E−10 |
| | | lysozyme gene | | | PROTEIN | |
| | | (cow 2), complete | | | | |
| | | cds | | | | |
| 411 | Z60048 | H. sapiens CpG | 4.00E−54 | HN3B_MOUSE | HEPATOCYTE | 4.00E−21 |
| | | DNA, clone | | | NUCLEAR FACTOR | |
| | | 187a9, reverse | | | 3-BETA (HNF-3B) | |
| | | read | | | | |
| | | cpg187a9.rt1a. | | | | |
| 412 | Z48975 | P. magnus gene | 0.014 | YPT2_CAEEL | HYPOTHETICAL | 2.00E−12 |
| | | for protein urPAB | | | 21.6 KD PROTEIN | |
| | | | | | F37A4.2 IN | |
| | | | | | CHROMOSOME III | |
| 413 | AJ001296 | Notophthalmus | 0.37 | YA53_SCHPO | HYPOTHETICAL | 5.00E−21 |
| | | viridescens | | | 24.2 KD PROTEIN | |
| | | mRNA for | | | C13A11.03 IN | |
| | | cytokeratin 8 | | | CHROMOSOME I | |
| 414 | J03831 | Xenopus laevis | 0.37 | PDR5_YEAST | SUPPRESSOR OF | 3.3 |
| | | (clone pXEC1.3) | | | TOXICITY OF | |
| | | C protein mRNA, | | | SPORIDESMIN | |
| | | complete cds. | | | | |
| 415 | AB007157 | Homo sapiens | e−142 | RS21_HUMAN | 40S RIBOSOMAL | 0.002 |
| | | gene for | | | PROTEIN S21 | |
| | | ribosomal protein | | | | |
| | | S21, partial cds | | | | |
| 416 | X86340 | H. sapiens C7 | 3.3 | STC_DROME | SHUTTLE CRAFT | 4.3 |
| | | gene, exon 13 | | | PROTEIN | |
| 417 | U12404 | Human Csa-19 | 0 | R10A_PIG | 60S RIBOSOMAL | 9.00E−57 |
| | | mRNA, complete | | | PROTEIN L10A | |
| | | cds. | | | (CSA-19) | |
| | | | | | (FRAGMENT) | |
| 418 | U95102 | Xenopus laevis | 8.00E−08 | <NONE> | <NONE> | <NONE> |
| | | mitotic | | | | |
| | | phosphoprotein | | | | |
| | | 90 mRNA, | | | | |
| | | complete cds | | | | |
| 419 | M80198 | Human FKBP-12 | 5.00E−14 | RCO1_NEUCR | TRANSCRIPTIONA | 0.008 |
| | | pseudogene, clone | | | L REPRESSOR RCO-1 | |
| | | lambda-512, 5′ | | | | |
| | | flank and | | | | |
| | | complete cds. | | | | |
| 420 | AF052573 | Homo sapiens | 0 | <NONE> | <NONE> | <NONE> |
| | | DNA polymerase | | | | |
| | | eta (POLH) | | | | |
| | | mRNA, complete | | | | |
| | | cds | | | | |
| 421 | AF035940 | Homo sapiens | e−131 | MGN_DROME | MAGO NASHI | 4.00E−39 |
| | | MAGOH mRNA, | | | PROTEIN | |
| | | complete cds | | | | |
| 422 | AF054994 | Homo sapiens | 0.12 | <NONE> | <NONE> | <NONE> |
| | | clone 23832 | | | | |
| | | mRNA sequence | | | | |
| 423 | U95098 | Xenopus laevis | 6.00E−05 | <NONE> | <NONE> | <NONE> |
| | | mitotic | | | | |
| | | phosphoprotein | | | | |
| | | 44 mRNA, partial | | | | |
| | | cds | | | | |
| 424 | U95094 | Xenopus laevis | 7.00E−07 | <NONE> | <NONE> | <NONE> |
| | | XL-INCENP | | | | |
| | | (XL-INCENP) | | | | |
| | | mRNA, complete | | | | |
| | | cds | | | | |
| 425 | D43952 | Mouse gene for | 0.36 | <NONE> | <NONE> | <NONE> |
| | | reticulocalbin, | | | | |
| | | exon 1 and | | | | |
| | | promoter region | | | | |
| 426 | X68553 | C. elegans | 0.4 | TCB1_RABIT | T-CELL RECEPTOR | 0.11 |
| | | repetitive DNA | | | BETA CHAIN | |
| | | sequence | | | PRECURSOR (ANA | |
| | | | | | 11) | |
| 427 | M83314 | Tomato | 3.3 | SMB2_HUMAN | DNA-BINDING | 0.65 |
| | | phenylalanine | | | PROTEIN SMUBP-2 | |
| | | ammonia lyase | | | (GLIAL FACTOR-1) | |
| | | (pal) gene, | | | (GF-1) | |
| | | complete cds and | | | | |
| | | promoter region. | | | | |
| 428 | AF070636 | Homo sapiens | 5.00E−23 | <NONE> | <NONE> | <NONE> |
| | | clone 24686 | | | | |
| | | mRNA sequence | | | | |
| 429 | <NONE> | <NONE> | <NONE> | IQGA_HUMAN | RAS GTPASE− | 2.00E−06 |
| | | | | | ACTIVATING-LIKE | |
| | | | | | PROTEIN IQGAP1 | |
| | | | | | (P195) | |
| 430 | AF068627 | Mus musculus | 5.00E−04 | LOX1_LENCU | LIPOXYGENASE | 9.9 |
| | | DNA cytosine−5 | | | (EC 1.13.11.12) | |
| | | methyltransferase | | | | |
| | | 3B2 (Dnmt3b) | | | | |
| | | mRNA, | | | | |
| | | alternatively | | | | |
| | | spliced, complete | | | | |
| | | cds | | | | |
| 431 | AF020043 | Homo sapiens | 0 | YJH4_YEAST | HYPOTHETICAL | 4.00E−16 |
| | | chromosome− | | | 141.3 KD PROTEIN | |
| | | associated | | | IN SCP160-MRPL8 | |
| | | polypeptide | | | INTERGENIC | |
| | | | | | REGION | |
| 432 | K00046 | ross river virus | 0.12 | CUL2_HUMAN | CULLIN HOMOLOG | 7.4 |
| | | 26s subgenomic | | | 2 (CUL-2) | |
| | | rna and junction | | | | |
| | | region. | | | | |
| 433 | AF005664 | Homo sapiens | 0.005 | UL88_HCMVA | PROTEIN UL88 | 5.8 |
| | | properdin (PFC) | | | | |
| | | gene, complete | | | | |
| | | cds | | | | |
| 434 | Z70705 | H. sapiens mRNA | 2.00E−05 | PH87_YEAST | INORGANIC | 1.5 |
| | | (fetal brain cDNA | | | PHOSPHATE | |
| | | com5) | | | TRANSPORTER | |
| | | | | | PHO87 | |
| 435 | U29156 | Mus musculus | e−125 | EP15_HUMAN | EPIDERMAL | 1.00E−13 |
| | | eps15R mRNA, | | | GROWTH FACTOR | |
| | | complete cds. | | | RECEPTOR | |
| | | | | | SUBSTRATE | |
| | | | | | SUBSTRATE 15 | |
| | | | | | (PROTEIN EPS 15) | |
| | | | | | (AF-1P PROTEIN) | |
| 436 | AE000750 | Aquifex aeolicus | 0.37 | <NONE> | <NONE> | <NONE> |
| | | section 82 of 109 | | | | |
| | | of the complete | | | | |
| | | genome | | | | |
| 437 | U49169 | Dictyostelium | 0.12 | VCAP_HSV6U | MAJOR CAPSID | 5.6 |
| | | discoideum V- | | | PROTEIN (MCP) | |
| | | ATPase A subunit | | | | |
| | | (vatA) mRNA, | | | | |
| | | complete cds | | | | |
| 438 | AF032871 | Homo sapiens | 0.13 | WEE1_SCHPO | MITOSIS | 3.7 |
| | | uncoupling | | | INHIBITOR | |
| | | protein 3 (UCP3) | | | PROTEIN KINASE | |
| | | gene, exon 1 and | | | WEE1 (EC 2.7.1.-) | |
| | | partial exon 2 | | | | |
| 439 | AB000425 | Porcine DNA for | 4.00E−32 | <NONE> | <NONE> | <NONE> |
| | | endopeptidase | | | | |
| | | 24.16, exon 16 | | | | |
| | | and complete cds | | | | |
| 440 | U51037 | Mus musculus 11- | 0.04 | <NONE> | <NONE> | <NONE> |
| | | zinc-finger | | | | |
| | | transcription | | | | |
| | | factor | | | | |
| 441 | AF032456 | Homo sapiens | e−110 | <NONE> | <NONE> | <NONE> |
| | | ubiquitin | | | | |
| | | conjugating | | | | |
| | | enzyme G2 | | | | |
| 442 | AF009288 | Homo sapiens | 2.00E−14 | LMG1_HUMAN | LAMININ GAMMA- | 8.1 |
| | | clone HEB8 Cri- | | | 1 CHAIN | |
| | | du-chat region | | | PRECURSOR | |
| | | mRNA | | | (LAMININ B2 | |
| | | | | | CHAIN) | |
| 443 | AF024578 | Homo sapiens | 1.1 | <NONE> | <NONE> | <NONE> |
| | | type−1 protein | | | | |
| | | phosphatase | | | | |
| | | skeletal muscle | | | | |
| | | glycogen | | | | |
| | | targeting subunit | | | | |
| | | (PPP1R3) gene, | | | | |
| | | exon 4, and | | | | |
| | | complete cds | | | | |
| 444 | M24486 | Human prolyl 4- | 0 | DACHA | <NONE> | 4.00E−58 |
| | | hydroxylase alpha | | | | |
| | | subunit mRNA, | | | | |
| | | complete cds, | | | | |
| | | clone PA-11. | | | | |
| 445 | X96400 | P. tetraurelia | 0.37 | <NONE> | <NONE> | <NONE> |
| | | alpha-51D gene | | | | |
| 446 | <NONE> | <NONE> | <NONE> | <NONE> | <NONE> | <NONE> |
| 447 | X84996 | X. laevis mRNA | 0.12 | POL_MLVRD | POL POLYPROTEIN | 2.00E−08 |
| | | for selenocysteine | | | (PROTEASE (EC | |
| | | tRNA acting | | | 3.4.23.-); REVERSE | |
| | | factor (Staf) | | | TRANSCRIPTASE | |
| | | | | | (EC 2.7.7.49); | |
| | | | | | RIBONUCLEASE H | |
| | | | | | (EC 3.1.26.4)) | |
| 448 | AF019980 | Dictyostelium | 3.4 | HMDL_BRAFL | HOMEOBOX | 0.23 |
| | | discoideum ZipA | | | PROTEIN DLL | |
| | | (zipA) gene, | | | HOMOLOG | |
| | | partial cds | | | | |
| 449 | X78424 | D. carota (Queen | 0.38 | <NONE> | <NONE> | <NONE> |
| | | Anne's Lace) | | | | |
| | | Inv*Dc2 gene, | | | | |
| | | 3432 bp | | | | |
| 450 | <NONE> | <NONE> | <NONE> | <NONE> | <NONE> | <NONE> |
| 451 | X89886 | P. patens mRNA | 1.1 | CKR6_HUMAN | C-C CHEMOKINE | 9.9 |
| | | for 5- | | | RECEPTOR TYPE 6 | |
| | | aminolevulinate | | | (C-C CKR-6) (CCR6) | |
| | | dehydratase | | | | |
| 452 | U67471 | Methanococcus | 0.12 | YR72_ECOLI | HYPOTHETICAL | 5.8 |
| | | jannaschii section | | | 53.2 KD PROTEIN | |
| | | 13 of 150 of the | | | (ORF2) (RETRON | |
| | | complete genome | | | EC67) | |
| 453 | AF060246 | Mus musculus | 1.00E−62 | YOJ8_CAEEL | HYPOTHETICAL | 1.7 |
| | | strain C57BL/6 | | | 51.6 KD PROTEIN | |
| | | zinc finger protein | | | ZK353.8 IN | |
| | | 106 (Zfp106) | | | CHROMOSOME III | |
| | | mRNA, H3a-a | | | | |
| | | allele, complete | | | | |
| | | cds | | | | |
| 454 | U70667 | Human Fas-ligand | 0 | YKB2_YEAST | HYPOTHETICAL | 3.00E−09 |
| | | associated factor | | | 69.1 KD PROTEIN | |
| | | 1 mRNA, partial | | | IN PUT3-CCE1 | |
| | | cds | | | INTERGENIC | |
| | | | | | REGION | |
| 455 | M95858 | Bos taurus | 0.35 | GIDA_MYCGE | GLUCOSE | 1.4 |
| | | recoverin mRNA, | | | INHIBITED | |
| | | complete cds. | | | DIVISION PROTEIN | |
| | | | | | A | |
| 456 | U67594 | Methanococcus | 0.36 | <NONE> | <NONE> | <NONE> |
| | | jannaschii section | | | | |
| | | 136 of 150 of the | | | | |
| | | complete genome | | | | |
| 457 | X06747 | Human hnRNP | 3.00E−31 | <NONE> | <NONE> | <NONE> |
| | | core protein A1 | | | | |
| 458 | Z65575 | H. sapiens CpG | 1.3 | <NONE> | <NONE> | <NONE> |
| | | DNA, clone 47c5, | | | | |
| | | reverse read | | | | |
| | | cpg47c5.rt1a. | | | | |
| 459 | X88893 | C. jacchus intron 4 | 5.00E−15 | <NONE> | <NONE> | <NONE> |
| | | of visual pigment | | | | |
| | | gene | | | | |
| 460 | M57426 | Maize stripe virus | 0.33 | DSC2_MOUSE | DESMOCOLLIN | 6.5 |
| | | RNA3 | | | 2A/2B PRECURSOR | |
| | | nonstructural | | | (EPITHELIAL TYPE | |
| | | protein | | | 2 DESMOCOLLIN) | |
| 461 | X01638 | Yeast TEF1 gene | 1.1 | PPOL_DROME | POLY (ADP- | 3.5 |
| | | for elongation | | | RIBOSE) | |
| | | factor EF-1 alpha | | | POLYMERASE (EC | |
| | | | | | 2.4.2.30) (PARP) | |
| 462 | M60064 | S. typhimurium | 1.1 | EPB4_MOUSE | EPHRIN TYPE−B | 2.5 |
| | | glutamate 1- | | | RECEPTOR 4 | |
| | | semialdehyde | | | PRECURSOR (EC | |
| | | aminotransferase | | | 2.7.1.112) KINASE 2) | |
| | | (hemL) gene, | | | (TYROSINE | |
| | | complete cds. | | | KINASE MYK- 1) | |
| 463 | X51508 | Rabbit mRNA for | 0.36 | ACHG_XENLA | ACETYLCHOLINE | 1.5 |
| | | aminopeptidase N | | | RECEPTOR | |
| | | (partial) | | | PROTEIN, GAMMA | |
| | | | | | CHAIN | |
| | | | | | PRECURSOR | |
| 464 | L10106 | Mus musculus | 2.00E−58 | VG13_BPML5 | GENE 13 PROTEIN | 2.5 |
| | | protein tyrosine | | | (GP 13) | |
| | | phosphate | | | | |
| | | mRNA, complete | | | | |
| | | cds. | | | | |
| 465 | M77235 | Human cardiac | 3.8 | ZPBOC1 | <NONE> | 6.9 |
| | | tetrodotoxin- | | | | |
| | | insensitive | | | | |
| | | voltage−dependent | | | | |
| | | sodium channel | | | | |
| | | alpha subunit | | | | |
| | | (HH1) mRNA, | | | | |
| | | complete cds. | | | | |
| 466 | M58330 | C. maltosa | 0.004 | EPB4_MOUSE | EPHRIN TYPE−B | 2.4 |
| | | autonomously | | | RECEPTOR 4 | |
| | | replicating | | | PRECURSOR (EC | |
| | | sequence. | | | 2.7.1.112) KINASE 2) | |
| | | | | | (TYROSINE | |
| | | | | | KINASE MYK- 1) | |
| 467 | X51508 | Rabbit mRNA for | 0.35 | ACHG_XENLA | ACETYLCHOLINE | 2.4 |
| | | aminopeptidase N | | | RECEPTOR | |
| | | (partial) | | | PROTEIN, GAMMA | |
| | | | | | CHAIN | |
| | | | | | PRECURSOR | |
| 468 | L10106 | Mus musculus | 7.00E−59 | VGLI_PRVRI | GLYCOPROTEIN | 4.3 |
| | | protein tyrosine | | | GP63 PRECURSOR | |
| | | phosphate | | | | |
| | | mRNA, complete | | | | |
| | | cds. | | | | |
| 469 | U65939 | Azotobacter | 1.1 | TRUA_BACSP | Q45557 bacillus sp. | 0.001 |
| | | vinelandii GTPase | | | (strain ksm-64). trna | |
| | | (ftsA) gene, | | | pseudouridine | |
| | | partial cds, and | | | synthase a (ec | |
| | | ATP binding | | | 4.2.1.70) | |
| | | protein (ftsZ) | | | (pseudouridylate | |
| | | gene, complete | | | synthase i) | |
| | | cds | | | (pseudouridine | |
| | | | | | synthase i) (uracil | |
| | | | | | hydrolyase). 11/98 | |
| 470 | U51037 | Mus musculus 11- | 0.041 | <NONE > | <NONE> | <NONE> |
| | | zinc-finger | | | | |
| | | transcription | | | | |
| | | factor | | | | |
| 471 | M32685 | Human platelet | 3.6 | <NONE> | <NONE> | <NONE> |
| | | glycoprotein IIIa, | | | | |
| | | exon 14. | | | | |
| 472 | U82691 | Phrynocephalus | 1.1 | <NONE> | <NONE> | <NONE> |
| | | raddei CAS | | | | |
| | | 179770 NADH | | | | |
| | | dehydrogenase | | | | |
| | | subunit 1 (ND1), | | | | |
| | | partial cds, tRNA- | | | | |
| | | Gln, tRNA-Ile | | | | |
| | | and tRNA-Met, | | | | |
| | | NADH | | | | |
| | | dehydrogenase | | | | |
| | | subunit 2 tRNA- | | | | |
| | | Cys and tRNA- | | | | |
| | | Tyr and c... | | | | |
| 473 | D85430 | Mouse Murr1 | 0.12 | EPA5_CHICK | EPHRIN TYPE−A | 2.5 |
| | | mRNA, exon | | | RECEPTOR 5 | |
| | | | | | PRECURSOR (EC | |
| | | | | | 2.7.1.112) | |
| 474 | U20661 | Dictyostelium | 0.36 | YHL1_EBV | HYPOTHETICAL | 4.00E−04 |
| | | discoideum | | | BHLF1 PROTEIN | |
| | | unknown internal | | | | |
| | | repeat protein | | | | |
| | | gene, complete | | | | |
| | | cds, and unknown | | | | |
| | | orf1, orf2 and | | | | |
| | | orf3 genes, partial | | | | |
| | | cds | | | | |
| 475 | X56537 | Human novel | 0.04 | FA5_HUMAN | COAGULATION | 9.5 |
| | | homeobox mRNA | | | FACTOR V | |
| | | for a DNA | | | PRECURSOR | |
| | | binding protein | | | (ACTIVATED | |
| | | | | | PROTEIN C | |
| | | | | | COFACTOR) | |
| 476 | U32843 | Haemophilus | 5 | <NONE> | <NONE> | <NONE> |
| | | influenzae Rd | | | | |
| | | section 158 of 163 | | | | |
| | | of the complete | | | | |
| | | genome | | | | |
| 477 | U67554 | Methanococcus | 0.36 | <NONE> | <NONE> | <NONE> |
| | | jannaschii section | | | | |
| | | 96 of 150 of the | | | | |
| | | complete genome | | | | |
| 478 | AB004244 | Narke japonica | 1.1 | NIA1_ORYSA | NITRATE | 1.00E−07 |
| | | mRNA for Nj- | | | REDUCTASE 1 (EC | |
| | | synaphin 1b, | | | 1.6.6.1) (NR1) | |
| | | complete cds | | | | |
| 479 | AF075079 | Homo sapiens full | 1.00E−12 | <NONE> | <NONE> | <NONE> |
| | | length insert | | | | |
| | | cDNA YQ80A08 | | | | |
| 480 | AE000723 | Aquifex aeolicus | 1 | YKK0_YEAST | HYPOTHETICAL | 9.1 |
| | | section 55 of 109 | | | 67.5 KD PROTEIN | |
| | | of the complete | | | IN APE1/LAP4- | |
| | | genome | | | CWP1 INTERGENIC | |
| | | | | | REGION | |
| 481 | X73902 | H. sapiens mRNA | 0 | LMG2_HUMAN | LAMININ GAMMA- | 3.00E−93 |
| | | for nicein B2 | | | 2 CHAIN | |
| | | chain | | | PRECURSOR | |
| 482 | U95094 | Xenopus laevis | 3.00E−10 | P53_CRIGR | CELLULAR TUMOR | 5.7 |
| | | XL-INCENP | | | ANTIGEN P53 | |
| | | (XL-INCENP) | | | | |
| | | mRNA, complete | | | | |
| | | cds | | | | |
| 483 | AL010240 | Plasmodium | 1.2 | <NONE> | <NONE> | <NONE> |
| | | falciparum DNA | | | | |
| | | *** | | | | |
| | | SEQUENCING | | | | |
| | | IN PROGRESS | | | | |
| | | *** from contig | | | | |
| | | 4-64, complete | | | | |
| | | sequence | | | | |
| 484 | U49919 | Arabidopsis | 0.54 | YA53_SCHPO | HYPOTHETICAL | 6.00E−10 |
| | | thalian lupeol | | | 24.2 KD PROTEIN | |
| | | synthase mRNA, | | | C13A11.03 IN | |
| | | complete cds | | | CHROMOSOME I | |
| 485 | AF077618 | Homo sapiens | 0.39 | MYOD_MOUSE | MYOBLAST | 2.1 |
| | | p73 gene, exon 3 | | | DETERMINATION | |
| | | | | | PROTEIN 1 | |
| 486 | AF054994 | Homo sapiens | 0.13 | <NONE> | <NONE> | <NONE> |
| | | clone 23832 | | | | |
| | | mRNA sequence | | | | |
| 487 | U95102 | Xenopus laevis | 3.00E−10 | <NONE> | <NONE> | <NONE> |
| | | mitotic | | | | |
| | | phosphoprotein | | | | |
| | | 90 mRNA, | | | | |
| | | complete cds | | | | |
| 488 | AF068627 | Mus musculus | 5.00E−04 | ACE2_YEAST | METALLOTHIONEI | 1.5 |
| | | DNA cytosine−5 | | | N EXPRESSION | |
| | | methyltransferase | | | ACTIVATOR | |
| | | 3B2 (Dnmt3b) | | | | |
| | | mRNA, | | | | |
| | | alternatively | | | | |
| | | spliced, complete | | | | |
| | | cds | | | | |
| 489 | U95102 | Xenopus laevis | 3.00E−07 | RINI_PIG | RIBONUCLEASE | 0.19 |
| | | mitotic | | | INHIBITOR | |
| | | phosphoprotein | | | | |
| | | 90 mRNA, | | | | |
| | | complete cds | | | | |
| 490 | L77886 | Human protein | 1.00E−21 | VS48_TBRVS | SATELLITE RNA 48 | 1.6 |
| | | tyrosine | | | KD PROTEIN | |
| | | phosphatase | | | | |
| | | mRNA, complete | | | | |
| | | cds | | | | |
| 491 | U95098 | Xenopus laevis | 5.00E−04 | CRP3_LIMPO | C-REACTIVE | 3.5 |
| | | mitotic | | | PROTEIN 3.3 | |
| | | phosphoprotein | | | PRECURSOR | |
| | | 44 mRNA, partial | | | | |
| | | cds | | | | |
| 492 | U95094 | Xenopus laevis | 8.00E−08 | EPA5_CHICK | EPHRIN TYPE−A | 2.7 |
| | | XL-INCENP | | | RECEPTOR 5 | |
| | | (XL-INCENP) | | | PRECURSOR (EC | |
| | | mRNA, complete | | | 2.7.1.112) | |
| | | cds | | | | |
| 493 | U95094 | Xenopus laevis | 3.00E−09 | <NONE> | <NONE> | <NONE> |
| | | XL-INCENP | | | | |
| | | (XL-INCENP) | | | | |
| | | mRNA, complete | | | | |
| | | cds | | | | |
| 494 | U28153 | Caenorhabditis | 0.37 | <NONE> | <NONE> | <NONE> |
| | | elegans UNC-76 | | | | |
| | | (unc-76) gene, | | | | |
| | | complete cds. | | | | |
| 495 | U95094 | Xenopus laevis | 0.37 | NCPR_YEAST | NADPH- | 7.00E−05 |
| | | XL-INCENP | | | CYTOCHROME | |
| | | (XL-INCENP) | | | P450 REDUCTASE | |
| | | mRNA, complete | | | (EC 1.6.2.4) (CPR) | |
| | | cds | | | | |
| 496 | U95102 | Xenopus laevis | 0.013 | YMB3_CAEEL | PROBABLE | 3.3 |
| | | mitotic | | | INTEGRIN ALPHA | |
| | | phosphoprotein | | | CHAIN F54G8.3 | |
| | | 90 mRNA, | | | PRECURSOR | |
| | | complete cds | | | | |
| 497 | U95102 | Xenopus laevis | 7.00E−07 | <NONE> | <NONE> | <NONE> |
| | | mitotic | | | | |
| | | phosphoprotein | | | | |
| | | 90 mRNA, | | | | |
| | | complete cds | | | | |
| 498 | U95094 | Xenopus laevis | 1.00E−10 | <NONE> | <NONE> | <NONE> |
| | | XL-INCENP | | | | |
| | | (XL-INCENP) | | | | |
| | | mRNA, complete | | | | |
| | | cds | | | | |
| 499 | U95102 | Xenopus laevis | 2.00E−07 | VGLY_LYCVW | GLYCOPROTEIN | 3.2 |
| | | mitotic | | | POLYPROTEIN | |
| | | phosphoprotein | | | PRECURSOR | |
| | | 90 mRNA, | | | (CONTAINS: | |
| | | complete cds | | | GLYCOPROTEINS | |
| | | | | | G1 AND G2) | |
| 500 | U95098 | Xenopus laevis | 8.00E−06 | HR78_DROME | NUCLEAR | 2.5 |
| | | mitotic | | | HORMONE | |
| | | phosphoprotein | | | RECEPTOR HR78 | |
| | | 44 mRNA, partial | | | (DHR78) (NUCLEAR | |
| | | cds | | | RECEPTOR | |
| | | | | | XR78E/F) | |
| 501 | U95102 | Xenopus laevis | 9.00E−10 | MYSH_BOVIN | MYOSIN I HEAVY | 4.00E−04 |
| | | mitotic | | | CHAIN-LIKE | |
| | | phosphoprotein | | | PROTEIN (MIHC) | |
| | | 90 mRNA, | | | (BRUSH BORDER | |
| | | complete cds | | | MYOSIN I) (BBMI) | |
| 502 | U95094 | Xenopus laevis | 2.00E−04 | BAL_HUMAN | BILE−SALT- | 2.6 |
| | | XL-INCENP | | | ACTIVATED | |
| | | (XL-INCENP) | | | LIPASE | |
| | | mRNA, complete | | | PRECURSOR (EC | |
| | | cds | | | 3.1.1.3) (EC 3.1.1.13) | |
| | | | | | (BAL) (BILE−SALT- | |
| | | | | | STIMULATED | |
| | | | | | LIPASE) (BSSL) | |
| | | | | | ESTERASE) | |
| | | | | | (PANCREATIC | |
| | | | | | LYSOPHOSPHOLIP | |
| | | | | | ASE) | |
| 503 | AF080399 | Drosophila | 1.1 | NAT1_YEAST | N-TERMINAL | 2.00E−23 |
| | | melanogaster | | | ACETYLTRANSFER | |
| | | mitotic | | | ASE 1 (EC 2.3.1.88) | |
| | | checkpoint | | | | |
| | | control protein | | | | |
| | | kinase BUB1 | | | | |
| | | (Bub1) mRNA, | | | | |
| | | complete cds | | | | |
| 504 | U59706 | Gallus gallus | 0.014 | <NONE> | <NONE> | <NONE> |
| | | alternatively | | | | |
| | | spliced AMPA | | | | |
| | | glutamate | | | | |
| | | receptor, isoform | | | | |
| | | GluR2 flop, | | | | |
| | | (GluR2) mRNA, | | | | |
| | | partial cds. | | | | |
| 505 | U95094 | Xenopus laevis | 2.00E−05 | <NONE> | <NONE> | <NONE> |
| | | XL-INCENP | | | | |
| | | (XL-INCENP) | | | | |
| | | mRNA, complete | | | | |
| | | cds | | | | |
| 506 | U95098 | Xenopus laevis | 2.00E−04 | <NONE> | <NONE> | <NONE> |
| | | mitotic | | | | |
| | | phosphoprotein | | | | |
| | | 44 mRNA, partial | | | | |
| | | cds | | | | |
| 507 | AF100661 | Caenorhabditis | 0.38 | <NONE> | <NONE> | <NONE> |
| | | elegans cosmid | | | | |
| | | H20E11 | | | | |
| 508 | U95102 | Xenopus laevis | 3.00E−11 | CA1A_HUMAN | COLLAGEN ALPHA | 0.024 |
| | | mitotic | | | 1(X) CHAIN | |
| | | phosphoprotein | | | PRECURSOR | |
| | | 90 mRNA, | | | | |
| | | complete cds | | | | |
| 509 | U47322 | Cloning vector | 2.00E−38 | COA1_SV40 | COAT PROTEIN | 6.2 |
| | | DNA, complete | | | VP1 | |
| | | sequence. | | | | |
| 510 | AF031924 | Homo sapiens | e−156 | CCMA_HAEIN | HEME EXPORTER | 3.5 |
| | | homeobox | | | PROTEIN A | |
| | | transcription | | | (CYTOCHROME C- | |
| | | factor barx2 | | | TYPE BIOGENESIS | |
| | | | | | ATP-BINDING | |
| | | | | | PROTEIN CCMA) | |
| 511 | AF010484 | Homo sapiens ICI | 3.00E−10 | <NONE> | <NONE> | <NONE> |
| | | YAC 9IA12, right | | | | |
| | | end sequence | | | | |
| 512 | Z63829 | H. sapiens CpG | 5.00E−22 | NFIR_MESAU | NUCLEAR FACTOR | 2.4 |
| | | DNA, clone 90h2, | | | 1 CLONE | |
| | | forward read | | | PNF1/RED1 (NF-I) | |
| | | cpg90h2.ft1a. | | | (CCAAT-BOX | |
| | | | | | BINDING | |
| | | | | | TRANSCRIPTION | |
| | | | | | FACTOR) (CTF) | |
| | | | | | (TGGCA-BINDING | |
| | | | | | PROTEIN) | |
| 513 | Z35094 | H. sapiens mRNA | 5.00E−97 | SUR2_HUMAN | SURFEIT LOCUS | 1.00E−46 |
| | | for SURF-2 | | | PROTEIN 2 | |
| 514 | U95102 | Xenopus laevis | 7.00E−06 | <NONE> | <NONE> | <NONE> |
| | | mitotic | | | | |
| | | phosphoprotein | | | | |
| | | 90 mRNA, | | | | |
| | | complete cds | | | | |
| 515 | D38417 | Mouse mRNA for | e−154 | TEGU_EBV | LARGE TEGUMENT | 3.4 |
| | | arylhydrocarbon | | | PROTEIN | |
| | | receptor, | | | | |
| | | complete cds | | | | |
| 516 | L10911 | Homo sapiens | e−117 | <NONE> | <NONE> | <NONE> |
| | | splicing factor | | | | |
| | | (CC1.4) mRNA, | | | | |
| | | complete cds. | | | | |
| 517 | X17093 | Human HLA-F | 0.009 | YEN1_SCHPO | O13695 | 5.4 |
| | | gene for human | | | schizosaccharomyces | |
| | | leukocyte antigen F | | | pombe (fission yeast). | |
| | | | | | hypothetical 52.9 kd | |
| | | | | | serine−rich protein | |
| | | | | | c11g7.01 in | |
| | | | | | chromosome i. 11/98 | |
| 518 | AB017026 | Mus musculus | 0 | OXYB_HUMAN | OXYSTEROL- | 1.00E−40 |
| | | mRNA for | | | BINDING PROTEIN | |
| | | oxysterol-binding | | | | |
| | | protein, complete | | | | |
| | | cds | | | | |
| 519 | X55038 | Mouse mCENP-B | 0.001 | YNW7_YEAST | HYPOTHETICAL | 3.00E−04 |
| | | gene for | | | 68.8 KD PROTEIN | |
| | | centromere | | | IN URE2-SSU72 | |
| | | autoantigen B | | | INTERGENIC | |
| | | | | | REGION | |
| 520 | AB018323 | Homo sapiens | 3.00E−41 | LBR_CHICK | LAMIN B | 2.3 |
| | | mRNA for | | | RECEPTOR | |
| | | KIAA0780 | | | | |
| | | protein, partial | | | | |
| | | cds | | | | |
| 521 | U95094 | Xenopus laevis | 1.00E−10 | CA25_HUMAN | PROCOLLAGEN | 0.002 |
| | | XL-INCENP | | | ALPHA 2(V) CHAIN | |
| | | (XL-INCENP) | | | PRECURSOR | |
| | | mRNA, complete | | | | |
| | | cds | | | | |
| 522 | X03558 | Human mRNA | 0 | EF11_HUMAN | ELONGATION | e−110 |
| | | for elongation | | | FACTOR 1-ALPHA 1 | |
| | | factor 1 alpha | | | (EF-1-ALPHA-1) | |
| | | subunit | | | | |
| 523 | U95102 | Xenopus laevis | 3.00E−11 | YMT8_YEAST | HYPOTHETICAL | 8.00E−07 |
| | | mitotic | | | 36.4 KD PROTEIN | |
| | | phosphoprotein | | | IN NUP116-FAR3 | |
| | | 90 mRNA, | | | INTERGENIC | |
| | | complete cds | | | REGION | |
| 524 | AB014591 | Homo sapiens | 0 | NOT2_YEAST | GENERAL | 8.00E−05 |
| | | mRNA for | | | NEGATIVE | |
| | | KIAA0691 | | | REGULATOR OF | |
| | | protein, complete | | | TRANSCRIPTION | |
| | | cds | | | SUBUNIT 2 | |
| 525 | AB019488 | Homo sapiens | 0 | TRKA_HUMAN | HIGH AFFINITY | 2.00E−27 |
| | | DNA for TRKA, | | | NERVE GROWTH | |
| | | exon 17 and | | | FACTOR | |
| | | complete cds | | | RECEPTOR | |
| | | | | | PRECURSOR | |
| | | | | | PROTEIN) (P140- | |
| | | | | | TRKA) | |
| 526 | U95102 | Xenopus laevis | 5.00E−15 | CNG4_BOVIN | 240K PROTEIN OF | 0.018 |
| | | mitotic | | | ROD | |
| | | phosphoprotein | | | PHOTORECEPTOR | |
| | | 90 mRNA, | | | CNG-CHANNEL | |
| | | complete cds | | | CYCLIC- | |
| | | | | | NUCLEOTIDE− | |
| | | | | | GATED CATION | |
| | | | | | CHANNEL 4 (CNG | |
| | | | | | CHANNEL 4) | |
| | | | | | MODULATORY | |
| | | | | | SUBUNIT)) | |
| 527 | U95094 | Xenopus laevis | 2.00E−06 | HMZ1_DROME | ZERKNUELLT | 0.88 |
| | | XL-INCENP | | | PROTEIN 1 (ZEN-1) | |
| | | (XL-INCENP) | | | | |
| | | mRNA, complete | | | | |
| | | cds | | | | |
| 528 | J03750 | Mouse single | e−135 | P15_HUMAN | ACTIVATED RNA | 3.00E−21 |
| | | stranded DNA | | | POLYMERASE II | |
| | | binding protein p9 | | | TRANSCRIPTIONA | |
| | | mRNA, complete | | | L COACTIVATOR | |
| | | cds. | | | P15 (PC4) (P14) | |
| 529 | U95094 | Xenopus laevis | 1.00E−12 | RS5_DROME | 40S RIBOSOMAL | 0.42 |
| | | XL-INCENP | | | PROTEIN S5 | |
| | | (XL-INCENP) | | | | |
| | | mRNA, complete | | | | |
| | | cds | | | | |
| 530 | Z57610 | H. sapiens CpG | 8.00E−61 | HN3B_MOUSE | HEPATOCYTE | 4.00E−15 |
| | | DNA, clone | | | NUCLEAR FACTOR | |
| | | 187a10, reverse | | | 3-BETA (HNF-3B) | |
| | | read | | | | |
| | | cpg187a10.rt1a. | | | | |
| 531 | U95760 | Drosophila | 3.00E−60 | <NONE> | <NONE> | <NONE> |
| | | melanogaster | | | | |
| | | strawberry notch | | | | |
| | | (sno) mRNA, | | | | |
| | | complete cds | | | | |
| 532 | U95094 | Xenopus laevis | 4.00E−11 | <NONE> | <NONE> | <NONE> |
| | | XL-INCENP | | | | |
| | | (XL-INCENP) | | | | |
| | | mRNA, complete | | | | |
| | | cds | | | | |
| 533 | U50535 | Human BRCA2 | 4.00E−12 | ALU1_HUMAN | !!!ALU | 1.1 |
| | | region, mRNA | | | SUBFAMILY J | |
| | | sequence CG006 | | | WARNING ENTRY | |
| | | | | | !!! | |
| 534 | X92841 | H. sapiens MICA | 1.00E−55 | LIN1_HUMAN | LINE−1 REVERSE | 6.00E−09 |
| | | gene | | | TRANSCRIPTASE | |
| | | | | | HOMOLOG | |
| 535 | U60337 | Homo sapiens | 0 | NODC_BRAEL | N- | 1.4 |
| | | beta-mannosidase | | | ACETYLGLUCOSA | |
| | | mRNA, complete | | | MINYLTRANSFERA | |
| | | cds | | | SE (EC 2.4.1.-) | |
| 536 | M21731 | Human lipocortin- | e−169 | ANX5_HUMAN | ANNEXIN V | 1.00E−05 |
| | | V mRNA, | | | (LIPOCORTIN V) | |
| | | complete cds. | | | (ENDONEXIN II) | |
| | | | | | (CALPHOBINDIN I) | |
| | | | | | (CBP-I) | |
| | | | | | (PLACENTAL | |
| | | | | | ANTICOAGULANT | |
| | | | | | PROTEIN I) (PAP-I) | |
| | | | | | ANTICOAGULANT- | |
| | | | | | ALPHA) (VAC- | |
| | | | | | ALPHA) | |
| | | | | | (ANCHORIN CII) | |
| 537 | Y08013 | S. salar DNA | 0.006 | <NONE> | <NONE> | <NONE> |
| | | segment | | | | |
| | | containing GT | | | | |
| | | repeat | | | | |
| 538 | <NONE> | <NONE> | <NONE> | <NONE> | <NONE> | <NONE> |
| 539 | M98502 | Mus musculus | 2.00E−17 | DYNA_CHICK | DYNACTIN, 117 KD | 7.4 |
| | | protein encoding | | | ISOFORM | |
| | | twelve zinc finger | | | | |
| | | proteins (pMLZ- | | | | |
| | | 4) mRNA, | | | | |
| | | complete cds. | | | | |
| 540 | U95102 | Xenopus laevis | 6.00E−05 | HXA3_HAEIN | HEME:HEMOPEXIN | 2.6 |
| | | mitotic | | | -BINDING PROTEIN | |
| | | phosphoprotein | | | PRECURSOR | |
| | | 90 mRNA, | | | | |
| | | complete cds | | | | |
| 541 | U95094 | Xenopus laevis | 1.00E−13 | AMO_KLEAE | AMINE OXIDASE | 1.5 |
| | | XL-INCENP | | | PRECURSOR (EC | |
| | | (XL-INCENP) | | | 1.4.3.6) | |
| | | mRNA, complete | | | (MONAMINE | |
| | | cds | | | OXIDASE) | |
| | | | | | (TYRAMINE | |
| | | | | | OXIDASE) | |
| 542 | AF083322 | Homo sapiens | e−133 | CA34_HUMAN | PROCOLLAGEN | 1.5 |
| | | centriole | | | ALPHA 3(IV) | |
| | | associated protein | | | CHAIN | |
| | | CEP110 mRNA, | | | PRECURSOR | |
| | | complete cds | | | | |
| 543 | J03746 | Human | e−170 | GTMI_HUMAN | GLUTATHIONES- | 5.00E−39 |
| | | glutathione S- | | | TRANSFERASE, | |
| | | transferase | | | MICROSOMAL (EC | |
| | | mRNA, complete | | | 2.5.1.18) | |
| | | cds. | | | | |
| 544 | U67522 | Methanococcus | 0.37 | A1AA_HUMAN | ALPHA-1A | 4.3 |
| | | jannaschii section | | | ADRENERGIC | |
| | | 64 of 150 of the | | | RECEPTOR | |
| | | complete genome | | | | |
| 545 | U95102 | Xenopus laevis | 2.00E−07 | <NONE> | <NONE> | <NONE> |
| | | mitotic | | | | |
| | | phosphoprotein | | | | |
| | | 90 mRNA, | | | | |
| | | complete cds | | | | |
| 546 | <NONE> | <NONE> | <NONE> | <NONE> | <NONE> | <NONE> |
| 547 | <NONE> | <NONE> | <NONE> | <NONE> | <NONE> | <NONE> |
| 548 | D87001 | Human (lambda) | 0.35 | VAL3_TYLCU | AL3 PROTEIN (C3 | 3.2 |
| | | DNA for | | | PROTEIN) | |
| | | immunoglobulin | | | | |
| | | light chain | | | | |
| 549 | U95094 | Xenopus laevis | 3.00E−08 | TEGU_HSV11 | LARGE TEGUMENT | 0.004 |
| | | XL-INCENP | | | PROTEIN (VIRION | |
| | | (XL-INCENP) | | | PROTEIN UL36) | |
| | | mRNA, complete | | | | |
| | | cds | | | | |
| 550 | D16991 | Human HepG2 | 8.00E−09 | PTM1_YEAST | PROTEIN PTM1 | 0.033 |
| | | partial cDNA, | | | PRECURSOR | |
| | | clone | | | | |
| | | hmd2d01m5 | | | | |
| 551 | M34025 | Human fetal Ig | 3.2 | <NONE> | <NONE> | <NONE> |
| | | heavy chain | | | | |
| | | variable region | | | | |
| 552 | M98502 | Mus musculus | 5.00E−14 | <NONE> | <NONE> | <NONE> |
| | | protein encoding | | | | |
| | | twelve zinc finger | | | | |
| | | proteins (pMLZ- | | | | |
| | | 4) mRNA, | | | | |
| | | complete cds. | | | | |
| 553 | U95098 | Xenopus laevis | 0.002 | <NONE> | <NONE> | <NONE> |
| | | mitotic | | | | |
| | | phosphoprotein | | | | |
| | | 44 mRNA, partial | | | | |
| | | cds | | | | |
| 554 | Z78730 | H. sapiens flow- | 3.00E−20 | ALU1_HUMAN | !!!ALU | 5.00E−06 |
| | | sorted | | | SUBFAMILY J | |
| | | chromosome 6 | | | WARNING ENTRY | |
| | | HindIII fragment, | | | !!! | |
| | | SC6pA15C3 | | | | |
| 555 | U74496 | Human | 8.00E−08 | ICP4_VZVD | TRANS-ACTING | 0.39 |
| | | chromosome 4q35 | | | TRANSCRIPTIONA | |
| | | subtelomeric | | | L PROTEIN ICP4 | |
| | | sequence | | | | |
| 556 | U39875 | Rattus norvegicus | 2.00E−56 | YHFK_ECOLI | HYPOTHETICAL | 9.8 |
| | | EF-hand Ca2'0 - | | | 79.5 KD PROTEIN | |
| | | binding protein | | | IN CRP-ARGD | |
| | | p22 mRNA, | | | INTERGENIC | |
| | | complete cds. | | | REGION (O696) | |
| 557 | U65416 | Human MHC | 0.12 | <NONE> | <NONE> | <NONE> |
| | | class I molecule | | | | |
| | | (MICB) gene, | | | | |
| | | complete cds | | | | |
| 558 | AG000037 | Homo sapiens | 5.00E−25 | <NONE> | <NONE> | <NONE> |
| | | genomic DNA, | | | | |
| | | 21q region, clone: | | | | |
| | | 9H11A22 | | | | |
| 559 | U95102 | Xenopus laevis | 5.00E−05 | <NONE> | <NONE> | <NONE> |
| | | mitotic | | | | |
| | | phosphoprotein | | | | |
| | | 90 mRNA, | | | | |
| | | complete cds | | | | |
| 560 | AB007918 | Homo sapiens | 0.015 | VGLE_HSV11 | GLYCOPROTEIN E | 2.2 |
| | | mRNA for | | | PRECURSOR | |
| | | KIAA0449 | | | | |
| | | protein, partial | | | | |
| | | cds | | | | |
| 561 | U58884 | Mus musculus | 1.00E−73 | YCV2_YEAST | HYPOTHETICAL | 2.6 |
| | | SH3-containing | | | 13.8 KD PROTEIN | |
| | | protein SH3P7 | | | IN PWP2-SUP61 | |
| | | mRNA, complete | | | INTERGENIC | |
| | | cds. similar to | | | REGION | |
| | | Human Drebrin | | | | |
| 562 | AB007878 | Homo sapiens | e−110 | GLU2_MAIZE | GLUTELIN 2 | 0.72 |
| | | KIAA0418 | | | PRECURSOR (ZEIN- | |
| | | mRNA, complete | | | GAMMA) (27 KD | |
| | | cds | | | ZEIN) | |
| 563 | AF065482 | Homo sapiens | 0 | YJD6_YEAST | HYPOTHETICAL | 1.4 |
| | | sorting nexin 2 | | | 49.0 KD PROTEIN | |
| | | (SNX2) mRNA, | | | IN NSP1-KAR2 | |
| | | complete cds | | | INTERGENIC | |
| | | | | | REGION | |
| 564 | U27873 | Stealth virus 1 | 0.002 | SYN1_HUMAN | SYNAPSINS IA | 1.6 |
| | | clone 3B11 T7 | | | AND IB (BRAIN | |
| | | | | | PROTEIN 4.1) | |
| 565 | L38951 | Homo sapiens | 2.00E−68 | VP2_BRD | STRUCTURAL | 1.1 |
| | | importin beta | | | CORE PROTEIN | |
| | | subunit mRNA, | | | VP2 | |
| | | complete cds | | | | |
| 566 | AF007155 | Homo sapiens | e−165 | YOHI_AZOVI | HYPOTHETICAL | 7.5 |
| | | clone 23763 | | | 33.2 KD PROTEIN | |
| | | unknown mRNA, | | | IN IBPB 5′ REGION | |
| | | partial cds | | | | |
| 567 | Z56295 | H. sapiens CpG | 0.12 | A1AB_CANFA | ALPHA-1B | 0.85 |
| | | DNA, clone 10c2, | | | ADRENERGIC | |
| | | forward read | | | RECEPTOR | |
| | | cpg10c2.ft1a. | | | (FRAGMENT) | |
| 568 | Z83792 | G. gallus | 0.12 | <NONE> | <NONE> | <NONE> |
| | | microsatellite | | | | |
| | | DNA (LEI0222 | | | | |
| 569 | U11820 | Feline | 1.1 | <NONE> | <NONE> | <NONE> |
| | | immunodeficienc | | | | |
| | | y virus | | | | |
| | | USIL2489_7B | | | | |
| | | gag polyprotein | | | | |
| | | (gag) gene, | | | | |
| | | complete cds, | | | | |
| | | polymerase | | | | |
| | | polyprotein (pol) | | | | |
| | | gene, partial cds, | | | | |
| | | vif protein (vif), | | | | |
| | | complete cds, and | | | | |
| | | envelope | | | | |
| | | glycoprotein | | | | |
| | | (env), complete | | | | |
| | | cds, complete g... | | | | |
| 570 | M18065 | Mouse 18S and | 6.00E−04 | CC40_YEAST | CELL DIVISION | 3.7 |
| | | 28S ribosomal | | | CONTROL | |
| | | DNA, 5′ | | | PROTEIN 40 | |
| | | hypervariable | | | | |
| | | (Vr) region, clone | | | | |
| | | M1. | | | | |
| 571 | AF053645 | Homo sapiens | 2.00E−07 | YMQ4_CAEEL | HYPOTHETICAL | 4.3 |
| | | cellular apoptosis | | | 25.8 KD PROTEIN | |
| | | susceptibility | | | K02D10.4 IN | |
| | | protein (CSE1) | | | CHROMOSOME III | |
| | | gene, exons 3 | | | | |
| | | through 10 | | | | |
| 572 | X04588 | Human 2.5 kb | 0 | <NONE> | <NONE> | <NONE> |
| | | mRNA for | | | | |
| | | cytoskeletal | | | | |
| | | tropomyosin | | | | |
| | | TM30(nm) | | | | |
| 573 | AC001159 | Homo sapiens | 5.00E−04 | XYND_CELFI | ENDO-1,4-BETA- | 7.3 |
| | | (subclone 1_h9 | | | XYLANASED | |
| | | from PAC H92) | | | PRECURSOR (EC | |
| | | DNA sequence | | | 3.2.1.8) | |
| 574 | Z60625 | H. sapiens CpG | 4.00E−13 | <NONE> | <NONE> | <NONE> |
| | | DNA, clone 2c10, | | | | |
| | | forward read | | | | |
| | | cpg2c10.ft1aa. | | | | |
| 575 | AF070640 | Homo sapiens | e−164 | <NONE> | <NONE> | <NONE> |
| | | clone 24781 | | | | |
| | | mRNA sequence | | | | |
| 576 | Y11306 | Homo sapiens | 2.00E−48 | TCF1_HUMAN | T-CELL-SPECIFIC | 2.00E−15 |
| | | mRNA for hTCF-4 | | | TRANSCRIPTION | |
| | | | | | FACTOR 1 (TCF-1) | |
| 577 | X65279 | pWE15 cosmid | 7.00E−69 | OCLN_POTTR | Q28793 potorous | 0.71 |
| | | vector DNA | | | tridactylus (potoroo). | |
| | | | | | occludin. 11/98 | |
| 578 | M10296 | Mouse DNA with | 0.001 | LMB1_HYDAT | LAMININ BETA-1 | 1.9 |
| | | homology to EBV | | | CHAIN | |
| | | IR3 repeat, | | | PRECURSOR | |
| | | segment 1, clone | | | (FRAGMENTS) | |
| | | Mu2. | | | | |
| 579 | X53744 | Canine mRNA for | e−162 | SR68_CANFA | SIGNAL | 5.00E−16 |
| | | 68 kDA subunit of | | | RECOGNITION | |
| | | signal recognition | | | PARTICLE 68 KD | |
| | | particle (SRP68) | | | PROTEIN (SRP68) | |
| 580 | AF086438 | Homo sapiens full | 2.00E−04 | <NONE> | <NONE> | <NONE> |
| | | length insert | | | | |
| | | cDNA clone | | | | |
| | | ZD80G11 | | | | |
| 581 | U15140 | Mycobacterium | 1.3 | <NONE> | <NONE> | <NONE> |
| | | bovis ribosomal | | | | |
| | | proteins IF-1 | | | | |
| | | complete cds, and | | | | |
| | | S4 (rpsD) gene, | | | | |
| | | partial cds | | | | |
| 582 | D13292 | Human mRNA | e−166 | RSP4_ARATH | 40S RIBOSOMAL | 1.4 |
| | | for ryudocan core | | | PROTEIN SA (P40) | |
| | | protein | | | (LAMININ | |
| | | | | | RECEPTOR | |
| | | | | | HOMOLOG) | |
| 583 | S71022 | neoplasm-related | 9.00E−30 | RL6_HUMAN | 60S RIBOSOMAL | 5.6 |
| | | C140 product | | | PROTEIN L6 (TAX- | |
| | | [human, thyroid | | | RESPONSIVE | |
| | | carcinoma cells, | | | ENHANCER | |
| | | mRNA, 670 nt] | | | ELEMENT BINDING | |
| | | | | | PROTEIN 107) | |
| | | | | | (TAXREB 107) | |
| 584 | L20934 | Anopheles | 0.014 | <NONE> | <NONE> | <NONE> |
| | | gambiae complete | | | | |
| | | mitochondrial | | | | |
| | | genome | | | | |
| 585 | Z49269 | H. sapiens gene | 1.1 | AMY1_DICTH | ALPHA-AMYLASE | 2.5 |
| | | for chemokine | | | 1 (EC 3.2.1.1) (1,4- | |
| | | HCC-1. | | | ALPHA-D-GLUCAN | |
| | | | | | GLUCANOHYDROL | |
| | | | | | ASE) | |
| 586 | U95098 | Xenopus laevis | 2.00E−04 | <NONE> | <NONE> | <NONE> |
| | | mitotic | | | | |
| | | phosphoprotein | | | | |
| | | 44 mRNA, partial | | | | |
| | | cds | | | | |
| 587 | AF029893 | Homo sapiens i- | 0.13 | HEMO_PIG | HEMOPEXIN | 3.5 |
| | | beta-1,3-N- | | | PRECURSOR | |
| | | acetylglucosamin | | | (HYALURONIDASE | |
| | | yltransferase | | | ) (EC 3.2.1.35) | |
| | | mRNA, complete | | | | |
| | | cds | | | | |
| 588 | J05109 | T. thermophila | 0.014 | <NONE> | <NONE> | <NONE> |
| | | calcium-binding | | | | |
| | | 25 kDa (TCBP | | | | |
| | | 25) protein gene, | | | | |
| | | complete cds. | | | | |
| 589 | U95098 | Xenopus laevis | 6.00E−04 | <NONE> | <NONE> | <NONE> |
| | | mitotic | | | | |
| | | phosphoprotein | | | | |
| | | 44 mRNA, partial | | | | |
| | | cds | | | | |
| 590 | AF060246 | Mus musculus | 1.00E−83 | SCRB_PEDPE | SUCROSE−6- | 10 |
| | | strain C57BL/6 | | | PHOSPHATE | |
| | | zinc finger protein | | | HYDROLASE (EC | |
| | | 106 (Zfp 106) | | | 3.2.1.26) (SUCRASE) | |
| | | mRNA, H3a-a | | | | |
| | | allele, complete | | | | |
| | | cds | | | | |
| 591 | Y11966 | B. aphidicola (host | 0.37 | <NONE> | <NONE> | <NONE> |
| | | T. suberi) plasmid | | | | |
| | | pBTs1 genes | | | | |
| | | leuA, hspA, | | | | |
| | | repA2, repA1, | | | | |
| | | leuB, leuC, leuD, | | | | |
| | | leuA | | | | |
| 592 | U20428 | Human SNC19 | 1.00E−64 | YY22_MYCTU | HYPOTHETICAL | 0.29 |
| | | mRNA sequence | | | 30.8 KD PROTEIN | |
| | | | | | CY49.22 | |
| 593 | AF043084 | Lycopersicon | 0.37 | KNIR_DROME | ZYGOTIC GAP | 9.9 |
| | | esculentum | | | PROTEIN KNIRPS | |
| | | ethylene receptor | | | | |
| | | homolog (ETR1) | | | | |
| | | mRNA, complete | | | | |
| | | cds | | | | |
| 594 | X65279 | pWE15 cosmid | 5.00E−66 | COA1_SV40 | COAT PROTEIN | 0.001 |
| | | vector DNA | | | VP1 | |
| 595 | U95098 | Xenopus laevis | 0.041 | UL88_HSV7J | PROTEIN U59 | 5.8 |
| | | mitotic | | | | |
| | | phosphoprotein | | | | |
| | | 44 mRNA, partial | | | | |
| | | cds | | | | |
| 596 | M91452 | Sus scrofa | 3.2 | <NONE> | <NONE> | <NONE> |
| | | ryanodine | | | | |
| | | receptor (RYR1) | | | | |
| | | gene, complete | | | | |
| | | cds. | | | | |
| 597 | U77327 | Human Ki-1/57 | e−158 | GAT1_CHICK | ERYTHROID | 1.2 |
| | | intracellular | | | TRANSCRIPTION | |
| | | antigen mRNA, | | | FACTOR (GATA-1) | |
| | | partial cds | | | (ERYF1) | |
| 598 | U77327 | Human Ki-1/57 | 0 | RPB7_ARATH | DNA-DIRECTED | 6.2 |
| | | intracellular | | | RNA POLYMERASE | |
| | | antigen mRNA, | | | II 19 KD | |
| | | partial cds | | | POLYPEPTIDE (EC | |
| | | | | | 2.7.7.6) (RNA | |
| | | | | | POLYMERASE II | |
| | | | | | SUBUNIT 5) | |
| 599 | Y16964 | Saccharomyces | 0.37 | NMD5_YEAST | NONSENSE− | 1.9 |
| | | sp. mitochondrial | | | MEDIATED MRNA | |
| | | DNA for OLI1 | | | DECAY PROTEIN 5 | |
| | | gene, strain CID1 | | | | |
| 600 | U95102 | Xenopus laevis | 6.00E−06 | <NONE> | <NONE> | <NONE> |
| | | mitotic | | | | |
| | | phosphoprotein | | | | |
| | | 90 mRNA, | | | | |
| | | complete cds | | | | |
| 601 | U95098 | Xenopus laevis | 8.00E−08 | <NONE> | <NONE> | <NONE> |
| | | mitotic | | | | |
| | | phosphoprotein | | | | |
| | | 44 mRNA, partial | | | | |
| | | cds | | | | |
| 602 | AF091046 | Brugia pahangi | 1.1 | INVO_PONPY | INVOLUCRIN | 0.23 |
| | | nuclear hormone | | | | |
| | | receptor (bhr-1) | | | | |
| | | gene, partial cds | | | | |
| 603 | M87339 | Human | 0 | AC12_HUMAN | ACTIVATOR 1 37 | 1.00E−38 |
| | | replication factor | | | KD SUBUNIT | |
| | | C, 37-kDa subunit | | | (REPLICATION | |
| | | mRNA, complete | | | FACTOR C 37 KD | |
| | | cds | | | SUBUNIT) (A1 37 | |
| | | | | | KD SUBUNIT) (RF- | |
| | | | | | C 37 KD SUBUNIT) | |
| | | | | | (RFC37) | |
| 604 | D28116 | Human genes for | 0.39 | <NONE> | <NONE> | <NONE> |
| | | collagen type IV | | | | |
| | | alpha 5 and 6, | | | | |
| | | exon 1 and exon | | | | |
| | | 1′ | | | | |
| 605 | U95102 | Xenopus laevis | 2.00E−06 | <NONE> | <NONE> | <NONE> |
| | | mitotic | | | | |
| | | phosphoprotein | | | | |
| | | 90 mRNA, | | | | |
| | | complete cds | | | | |
| 606 | AE001149 | Borrelia | 0.13 | <NONE> | <NONE> | <NONE> |
| | | burgdorferi | | | | |
| | | (section 35 of 70) | | | | |
| | | of the complete | | | | |
| | | genome | | | | |
| 607 | X14168 | Human pLC46 | 6.00E−16 | Z136_HUMAN | ZINC FINGER | 0.31 |
| | | with DNA | | | PROTEIN 136 | |
| | | replication origin | | | | |
| 608 | Z57610 | H. sapiens CpG | 7.00E−90 | HN3B_RAT | HEPATOCYTE | 1.00E−19 |
| | | DNA, clone | | | NUCLEAR FACTOR | |
| | | 187a10, reverse | | | 3-BETA (HNF-3B) | |
| | | read | | | | |
| | | cpg187a10.rt1a. | | | | |
| 609 | U95098 | Xenopus laevis | 0.043 | PGCV_MOUSE | VERSICAN CORE | 3.5 |
| | | mitotic | | | PROTEIN | |
| | | phosphoprotein | | | PRECURSOR | |
| | | 44 mRNA, partial | | | (LARGE | |
| | | cds | | | FIBROBLAST | |
| | | | | | PROTEOGLYCAN) | |
| | | | | | (CHONDROITIN | |
| | | | | | SULFATE | |
| | | | | | PROTEOGLYCAN | |
| | | | | | CORE PROTEIN 2) | |
| | | | | | (PG-M) | |
| 610 | U95094 | Xenopus laevis | 7.00E−07 | CA11_CHICK | PROCOLLAGEN | 0.4 |
| | | XL-INCENP | | | ALPHA 1(I) CHAIN | |
| | | (XL-INCENP) | | | PRECURSOR | |
| | | mRNA, complete | | | | |
| | | cds | | | | |
| 611 | AB007956 | Homo sapiens | e−106 | RRPB_CVMA5 | RNA-DIRECTED | 9.7 |
| | | mRNA, | | | RNA POLYMERASE | |
| | | chromosome 1 | | | (EC 2.7.7.48) | |
| | | specific transcript | | | (ORF1B) | |
| | | KIAA0487 | | | | |
| 612 | U95102 | Xenopus laevis | 0.005 | <NONE> | <NONE> | <NONE> |
| | | mitotic | | | | |
| | | phosphoprotein | | | | |
| | | 90 mRNA, | | | | |
| | | complete cds | | | | |
| 613 | U95094 | Xenopus laevis | 6.00E−05 | UL52_EBV | HELICASE/PRIMAS | 5.9 |
| | | XL-INCENP | | | E COMPLEX | |
| | | (XL-INCENP) | | | PROTEIN | |
| | | mRNA, complete | | | (PROBABLE DNA | |
| | | cds | | | REPLICATION | |
| | | | | | PROTEIN BSLF1) | |
| 614 | U95760 | Drosophila | 3.00E−71 | POLG_PVYHU | GENOME | 4.3 |
| | | melanogaster | | | POLYPROTEIN | |
| | | strawberry notch | | | (CONTAINS: N- | |
| | | (sno) mRNA, | | | TERMINAL | |
| | | complete cds | | | PROTEIN; HELPER | |
| | | | | | COMPONENT | |
| | | | | | PROTEINASE (EC | |
| | | | | | 3.4.22.-) (HC-PRO); | |
| | | | | | 42- 50 KD PROTEIN; | |
| | | | | | CYTOPLASMIC | |
| | | | | | INCLUSION | |
| | | | | | PROTEIN (CI); 6 KD | |
| | | | | | PROTEIN; | |
| | | | | | NUCLEAR | |
| | | | | | INCLUSION | |
| | | | | | PROTEIN A (NI-A) | |
| | | | | | (EC 3.4.22.-) (49K | |
| | | | | | PROTEINASE) (49 | |
| 615 | U95102 | Xenopus laevis | 9.00E−09 | VP3_ROTPC | INNER CORE | 7.7 |
| | | mitotic | | | PROTEIN VP3 | |
| | | phosphoprotein | | | | |
| | | 90 mRNA, | | | | |
| | | complete cds | | | | |
| 616 | J05499 | Rattus norvegicus | e−143 | GLSL_RAT | GLUTAMINASE, | 7.00E−67 |
| | | L-glutamine | | | LIVER ISOFORM | |
| | | amidohydrolase | | | PRECURSOR (EC | |
| | | mRNA, complete | | | 3.5.1.2) (GLS) | |
| | | cds | | | | |
| 617 | M19262 | Rat clathrin light | 0.37 | Y642_METJA | HYPOTHETICAL | 5.8 |
| | | chain (LCB3) | | | PROTEIN MJ0642 | |
| | | mRNA, complete | | | | |
| | | cds. | | | | |
| 618 | M21191 | Human aldolase | 1.00E−32 | LIN1_NYCCO | LINE−1 REVERSE | 6.00E−17 |
| | | pseudogene | | | TRANSCRIPTASE | |
| | | mRNA, complete | | | HOMOLOG | |
| | | cds. | | | | |
| 619 | U95094 | Xenopus laevis | 1.00E−11 | NUCM_BOVIN | NADH- | 0.044 |
| | | XL-INCENP | | | UBIQUINONE | |
| | | (XL-INCENP) | | | OXIDOREDUCTASE | |
| | | mRNA, complete | | | 49KD SUBUNIT (EC | |
| | | cds | | | 1.6.5.3) (EC 1.6.99.3) | |
| | | | | | (COMPLEX I-49KD) | |
| | | | | | (CI-49KD) | |
| 620 | U95098 | Xenopus laevis | 0.005 | HEMZ_RHOCA | FERROCHELATASE | 4.4 |
| | | mitotic | | | (EC 4.99.1.1) | |
| | | phosphoprotein | | | (PROTOHEME | |
| | | 44 mRNA, partial | | | FERRO-LYASE) | |
| | | cds | | | | |
| 621 | AF041428 | Homo sapiens | 0.002 | <NONE> | <NONE> | <NONE> |
| | | ribosomal protein | | | | |
| | | s4 X isoform | | | | |
| | | gene, complete | | | | |
| | | cds | | | | |
| 622 | X07158 | Chironomus | 0.13 | <NONE> | <NONE> | <NONE> |
| | | thummi DNA for | | | | |
| | | Cla repetitive | | | | |
| | | element | | | | |
| 623 | U95094 | Xenopus laevis | 8.00E−04 | <NONE> | <NONE> | <NONE> |
| | | XL-INCENP | | | | |
| | | (XL-INCENP) | | | | |
| | | mRNA, complete | | | | |
| | | cds | | | | |
| 624 | AF100470 | Rattus norvegicus | 1.00E−53 | <NONE> | <NONE> | <NONE> |
| | | ribosome attached | | | | |
| | | membrane protein | | | | |
| | | 4 (RAMP4) | | | | |
| | | mRNA, complete | | | | |
| | | cds | | | | |
| 625 | U85193 | Human nuclear | 2.00E−38 | <NONE> | <NONE> | <NONE> |
| | | factor I-B2 | | | | |
| | | (NFIB2) mRNA, | | | | |
| | | complete cds | | | | |
| 626 | M13452 | Human lamin A | 6.00E−16 | <NONE> | <NONE> | <NONE> |
| | | mRNA, 3′ end. | | | | |
| 627 | U95094 | Xenopus laevis | 0.014 | ACDV_RAT | ACYL-COA | 4.00E−20 |
| | | XL-INCENP | | | DEHYDROGENASE, | |
| | | (XL-INCENP) | | | VERY-LONG- | |
| | | mRNA, complete | | | CHAIN SPECIFIC | |
| | | cds | | | PRECURSOR (EC | |
| | | | | | 1.3.99.-) (VLCAD) | |
| 628 | U95094 | Xenopus laevis | 3.00E−10 | <NONE> | <NONE> | <NONE> |
| | | XL-INCENP | | | | |
| | | (XL-INCENP) | | | | |
| | | mRNA, complete | | | | |
| | | cds | | | | |
| 629 | <NONE> | <NONE> | <NONE> | <NONE> | <NONE> | <NONE> |
| 630 | U95102 | Xenopus laevis | 2.00E−05 | <NONE> | <NONE> | <NONE> |
| | | mitotic | | | | |
| | | phosphoprotein | | | | |
| | | 90 mRNA, | | | | |
| | | complete cds | | | | |
| 631 | U95102 | Xenopus laevis | 6.00E−05 | <NONE> | <NONE> | <NONE> |
| | | mitotic | | | | |
| | | phosphoprotein | | | | |
| | | 90 mRNA, | | | | |
| | | complete cds | | | | |
| 632 | U95094 | Xenopus laevis | 6.00E−05 | YS83_CAEEL | HYPOTHETICAL | 0.65 |
| | | XL-INCENP | | | 86.9 KD PROTEIN | |
| | | (XL-INCENP) | | | ZK945.3 IN | |
| | | mRNA, complete | | | CHROMOSOME II | |
| | | cds | | | | |
| 633 | U95102 | Xenopus laevis | 3.00E−09 | NRP_MOUSE | NEUROPILIN | 2.7 |
| | | mitotic | | | PRECURSOR (A5 | |
| | | phosphoprotein | | | PROTEIN) | |
| | | 90 mRNA, | | | | |
| | | complete cds | | | | |
| 634 | U95098 | Xenopus laevis | 2.00E−05 | Y4JN_RHISN | HYPOTHETICAL | 5.9 |
| | | mitotic | | | 16.3 KD PROTEIN | |
| | | phosphoprotein | | | Y4JN | |
| | | 44 mRNA, partial | | | | |
| | | cds | | | | |
| 635 | U95102 | Xenopus laevis | 6.00E−05 | <NONE> | <NONE> | <NONE> |
| | | mitotic | | | | |
| | | phosphoprotein | | | | |
| | | 90 mRNA, | | | | |
| | | complete cds | | | | |
| 636 | X64707 | H. sapiens BBC1 | e−179 | RL13_HUMAN | 60S RIBOSOMAL | 5.00E−40 |
| | | mRNA | | | PROTEIN L13 | |
| | | | | | (BREAST BASIC | |
| | | | | | CONSERVED | |
| | | | | | PROTEIN 1) | |
| 637 | U95102 | Xenopus laevis | 3.00E−08 | <NONE> | <NONE> | <NONE> |
| | | mitotic | | | | |
| | | phosphoprotein | | | | |
| | | 90 mRNA, | | | | |
| | | complete cds | | | | |
| 638 | X14168 | Human pLC46 | 5.00E−14 | SP3_HUMAN | TRANSCRIPTION | 0.19 |
| | | with DNA | | | FACTOR SP3 (SPR- | |
| | | replication origin | | | 2) (FRAGMENT) | |
| 639 | X90999 | H. sapiens mRNA | 9.00E−20 | GLO2_HUMAN | HYDROXYACYLGL | 0.007 |
| | | for Glyoxalase II | | | UTATHIONE | |
| | | | | | HYDROLASE (EC | |
| | | | | | 3.1.2.6) | |
| 640 | AF083322 | Homo sapiens | 9.00E−51 | KIF4_MOUSE | KINESIN-LIKE | 0.005 |
| | | centriole | | | PROTEIN KIF4 | |
| | | associated protein | | | | |
| | | CEP110 mRNA, | | | | |
| | | complete cds | | | | |
| 641 | Z12002 | M. musculus Pvt-1 | 0.36 | CP5F_CANTR | CYTOCHROME | 5.6 |
| | | mRNA. | | | P450 LIIA6 | |
| | | | | | (ALKANE− | |
| | | | | | INDUCIBLE) (EC | |
| | | | | | 1.14.14.1) (P450- | |
| | | | | | ALK3) | |
| 642 | M10206 | R. sphaeroides | 1.1 | YGR1_YEAST | HYPOTHETICAL | 0.006 |
| | | reaction center L | | | 34.8 KD PROTEIN | |
| | | subunit (complete | | | IN SUT1-RCK1 | |
| | | cds) and M | | | INTERGENIC | |
| | | subunit (5′ end) | | | REGION | |
| | | genes. | | | | |
| 643 | K02668 | E. coli ddl gene | 3.3 | ANKB_HUMAN | ANKYRIN, BRAIN | 7.00E−07 |
| | | encoding D- | | | VARIANT 1 | |
| | | alanine:D-alanine | | | (ANKYRIN B) | |
| | | ligase and ftsQ | | | (ANKYRIN, | |
| | | and ftsA genes, | | | NONERYTHROID) | |
| | | complete cds, and | | | | |
| | | ftsZ gene, 5′ end. | | | | |
| 644 | <NONE> | <NONE> | <NONE> | <NONE> | <NONE> | <NONE> |
| 645 | X53616 | C. domesticus | 1.1 | <NONE> | <NONE> | <NONE> |
| | | calnexin (pp90) | | | | |
| | | mRNA | | | | |
| 646 | X57010 | Human COL2A1 | 3.3 | PRIO_PIG | MAJOR PRION | 1.9 |
| | | gene for collagen | | | PROTEIN | |
| | | II alpha 1 chain, | | | PRECURSOR (PRP) | |
| | | exons E2-E15 | | | | |
| 647 | U95097 | Xenopus laevis | 1.1 | UL07_HSV2H | PROTEIN UL7 | 7.3 |
| | | mitotic | | | | |
| | | phosphoprotein | | | | |
| | | 43 mRNA, partial | | | | |
| | | cds | | | | |
| 648 | X52956 | Human CAMII- | 0.37 | PRTP_EBV | PROBABLE | 7.5 |
| | | psi3 calmodulin | | | PROCESSING AND | |
| | | retropseudogene | | | TRANSPORT | |
| | | | | | PROTEIN | |
| 649 | M93425 | Human protein | 0 | PTNC_HUMAN | PROTEIN- | e−107 |
| | | tyrosine | | | TYROSINE | |
| | | phosphatase | | | PHOSPHATASE G1 | |
| | | (PTP-PEST) | | | (EC 3.1.3.48) | |
| | | mRNA, complete | | | (PTPG1) | |
| | | cds. | | | | |
| 650 | L47615 | Mus musculus | 0.13 | YA53_SCHPO | HYPOTHETICAL | 2.00E−07 |
| | | DNA-binding | | | 24.2 KD PROTEIN | |
| | | protein (Fli-1) | | | C13A11.03 IN | |
| | | gene, 5′ end of | | | CHROMOSOME I | |
| | | cds. | | | | |
| 651 | U60337 | Homo sapiens | 0 | GIL1_ENTHI | GALACTOSE− | 0.22 |
| | | beta-mannosidase | | | INHIBITABLE | |
| | | mRNA, complete | | | LECTIN 170 KD | |
| | | cds | | | SUBUNIT | |
| 652 | U08813 | Oryctolagus | 1.00E−22 | NAG1_HUMAN | SODIUM/GLUCOSE | 0.1 |
| | | cuniculus | | | COTRANSPORTER | |
| | | Na+/glucose | | | 1 (NA(+)/GLUCOSE | |
| | | cotransporter- | | | COTRANSPORTER | |
| | | related protein | | | 1) (HIGH AFFINITY | |
| | | mRNA, complete | | | SODIUM-GLUCOSE | |
| | | cds. | | | COTRANSPORTER) | |
| 653 | Y00282 | Human mRNA | 2.00E−78 | RIB2_HUMAN | DOLICHYL- | 5.00E−19 |
| | | for ribophorin II | | | DIPHOSPHOOLIGO | |
| | | | | | SACCHARIDE− | |
| | | | | | PROTEIN | |
| | | | | | GLYCOSYLTRANS | |
| | | | | | FERASE 63 KD | |
| | | | | | SUBUNIT | |
| | | | | | PRECURSOR (EC | |
| | | | | | 2.4.1.119) | |
| | | | | | (RIBOPHORIN II) | |
| 654 | D10051 | Human gene for | 0.014 | TAGB_DICDI | PRESTALK- | 7.6 |
| | | 92-kDa type IV | | | SPECIFIC PROTEIN | |
| | | collagenase, 5′ - | | | TAGB PRECURSOR | |
| | | flanking region | | | (EC 3.4.21.-) | |
| 655 | M29930 | Human insulin | 8.00E−08 | <NONE> | <NONE> | <NONE> |
| | | receptor (allele 2) | | | | |
| | | gene, exons 14, | | | | |
| | | 15, 16 and 17. | | | | |
| 656 | U78310 | Homo sapiens | 0 | YG2S_YEAST | HYPOTHETICAL | 0.002 |
| | | pescadillo | | | 69.9 KD PROTEIN | |
| | | mRNA, complete | | | IN MIC1-SRB5 | |
| | | cds | | | INTERGENIC | |
| | | | | | REGION | |
| 657 | X68792 | S. coelicolor | 3.2 | YBS0_YEAST | HYPOTHETICAL | 0.073 |
| | | A3(2) promoter | | | 27.0 KD PROTEIN | |
| | | sequence pth270 | | | IN VAL1-HSP26 | |
| | | | | | INTERGENIC | |
| | | | | | REGION | |
| 658 | U50535 | Human BRCA2 | 4.00E−12 | ALU1_HUMAN | !!!! ALU | 1.2 |
| | | region, mRNA | | | SUBFAMILY J | |
| | | sequence CG006 | | | WARNING ENTRY | |
| | | | | | !!!! | |
| 659 | U15522 | Sus scrofa clone | 3.2 | Z165_HUMAN | ZINC FINGER | 3.2 |
| | | pvg1a Ig heavy | | | PROTEIN 165 | |
| | | chain variable | | | | |
| | | VDJ region | | | | |
| | | mRNA, partial | | | | |
| | | cds. | | | | |
| 660 | M20918 | C. thummi piger | 0.12 | YT25_CAEEL | HYPOTHETICAL | 0.033 |
| | | haemoglobin (Hb) | | | 59.9 KD PROTEIN | |
| | | gene DNA, | | | B0304.5 IN | |
| | | complete cds. | | | CHROMOSOME II | |
| 661 | U60337 | Homo sapiens | 0 | <NONE> | <NONE> | <NONE> |
| | | beta-mannosidase | | | | |
| | | mRNA, complete | | | | |
| | | cds | | | | |
| 662 | U95098 | Xenopus laevis | 0.001 | ENV_MLVFP | ENV POLYPROTEIN | 3.3 |
| | | mitotic | | | PRECURSOR | |
| | | phosphoprotein | | | (CONTAINS: KNOB | |
| | | 44 mRNA, partial | | | PROTEIN GP70; | |
| | | cds | | | SPIKE PROTEIN | |
| | | | | | P15E; R PROTEIN) | |
| 663 | M97287 | Human | 0 | SAT1_HUMAN | DNA-BINDING | 2.00E−20 |
| | | MAR/SAR DNA | | | PROTEIN SATB1 | |
| | | binding protein | | | (SPECIAL AT-RICH | |
| | | (SATB1) mRNA, | | | SEQUENCE | |
| | | complete cds.>:: | | | BINDING PROTEIN | |
| | | gb\|I58691\|I58691 | | | 1) | |
| | | Sequence 1 from | | | | |
| | | patent US | | | | |
| | | 5652340 | | | | |
| 664 | L42612 | Homo sapiens | e−168 | K2C4_BOVIN | KERATIN, TYPE II | 4.00E−10 |
| | | keratin 6 isoform | | | CYTOSKELETAL 59 | |
| | | K6f (KRT6F) | | | KD, COMPONENT | |
| | | mRNA, complete | | | IV | |
| | | cds | | | | |
| 665 | U17901 | Rattus norvegicus | e−152 | PLAP_MOUSE | PHOSPHOLIPASE | 4.00E−13 |
| | | phospholipase A- | | | A-2-ACTIVATING | |
| | | 2-activating | | | PROTEIN (PLAP) | |
| | | protein (plap) | | | | |
| | | mRNA, complete | | | | |
| | | cds. | | | | |
| 666 | M73047 | Homo sapiens | 0 | MERT_STRLI | MERCURIC | 4.4 |
| | | tripeptidyl | | | TRANSPORT | |
| | | peptidase II | | | PROTEIN | |
| | | mRNA, complete | | | (MERCURY ION | |
| | | cds. | | | TRANSPORT | |
| | | | | | PROTEIN) | |
| 667 | U09954 | Human ribosomal | 0 | RL9_HUMAN | 60S RIBOSOMAL | 2.00E−11 |
| | | protein L9 gene, | | | PROTEIN L9 | |
| | | 5′ region and | | | | |
| | | complete cds. | | | | |
| 668 | X98330 | H. sapiens mRNA | 1.1 | HS74_MOUSE | HEAT SHOCK 70 | 0.034 |
| | | for ryanodine | | | KD PROTEIN AGP-2 | |
| | | receptor 2 | | | | |
| 669 | U95094 | Xenopus laevis | 0.002 | RPC2_DROME | DNA-DIRECTED | 1.1 |
| | | XL-INCENP | | | RNA POLYMERASE | |
| | | (XL-INCENP) | | | III 128 KD | |
| | | mRNA, complete | | | POLYPEPTIDE | |
| | | cds | | | | |
| 670 | AF069250 | Homo sapiens | 7.00E−80 | LEGB_PEA | LEGUMIN B | 0.011 |
| | | okadaic acid- | | | (FRAGMENT) | |
| | | inducible | | | | |
| | | phosphoprotein | | | | |
| | | (OA48-18) | | | | |
| | | mRNA, complete | | | | |
| | | cds | | | | |
| 671 | Z71419 | S. cerevisiae | 1.1 | FOCD_ECOLI | OUTER | 9.7 |
| | | chromosome XIV | | | MEMBRANE | |
| | | reading frame | | | USHER PROTEIN | |
| | | ORF YNL143c | | | FOCD PRECURSOR | |
| 672 | AF044965 | Homo sapiens | e−167 | PVR_MOUSE | POLIOVIRUS | 1.00E−12 |
| | | polio virus related | | | RECEPTOR | |
| | | protein 2 gene, | | | HOMOLOG | |
| | | alpha isoform, | | | PRECURSOR | |
| | | exon 6 and partial | | | | |
| | | cds | | | | |
| 673 | X65319 | Cloning vector | 2.00E−80 | S106_HUMAN | CALCYCLIN | 3.00E−15 |
| | | pCAT-Enhancer | | | (PROLACTIN | |
| | | | | | RECEPTOR | |
| | | | | | ASSOCIATED | |
| | | | | | PROTEIN) | |
| | | | | | CALCIUM- | |
| | | | | | BINDING PROTEIN | |
| | | | | | A6) | |
| 674 | D29655 | Pig mRNA for | e−103 | V319_ASFB7 | J319 PROTEIN | 4.3 |
| | | UMP-CMP | | | | |
| | | kinase, complete | | | | |
| | | cds | | | | |
| 675 | U95094 | Xenopus laevis | 8.00E−08 | VEGR_RAT | VASCULAR | 3.3 |
| | | XL-INCENP | | | ENDOTHELIAL | |
| | | (XL-INCENP) | | | GROWTH FACTOR | |
| | | mRNA, complete | | | RECEPTOR 1 | |
| | | cds | | | PRECURSOR | |
| | | | | | RECEPTOR FLT) | |
| | | | | | (FLT-1) | |
| 676 | D90217 | S. cerevisiae gene | 2.00E−07 | MALY_ECOLI | MALY PROTEIN | 5.6 |
| | | for YmL33, | | | (EC 2.6.1.-) | |
| | | mitochondrial | | | | |
| | | ribosomal | | | | |
| | | proteins of large | | | | |
| | | subunit | | | | |
| 677 | AF038952 | Homo sapiens | e−160 | T1CA_MOUSE | TCP1-CHAPERONIN | 4.00E−19 |
| | | cofactor A protein | | | COFACTOR A | |
| | | mRNA, complete | | | | |
| | | cds | | | | |
| 678 | Z96950 | Gorilla gorilla | 5.00E−14 | YHBZ_ECOLI | HYPOTHETICAL | 3.3 |
| | | DNA sequence | | | 43.3 KD GTP- | |
| | | orthologous to the | | | BINDING PROTEIN | |
| | | human Xp:Yp | | | IN DACB-RPMA | |
| | | telomere−junction | | | INTERGENIC | |
| | | region | | | REGION (F390) | |
| 679 | D50418 | Mouse mRNA for | 2.00E−79 | CYGX_RAT | OLFACTORY | 1.1 |
| | | AREC3, partial | | | GUANYLYL | |
| | | cds | | | CYCLASE GC-D | |
| | | | | | PRECURSOR (EC | |
| | | | | | 4.6.1.2) | |
| 680 | U95098 | Xenopus laevis | 8.00E−08 | P2C2_SCHPO | PROTEIN | 1.00E−04 |
| | | mitotic | | | PHOSPHATASE 2C | |
| | | phosphoprotein | | | HOMOLOG 2 (EC | |
| | | 44 mRNA, partial | | | 3.1.3.16) | |
| | | cds | | | | |
| 681 | AL010280 | Plasmodium | 0.12 | <NONE> | <NONE> | <NONE> |
| | | falciparum DNA | | | | |
| | | *** | | | | |
| | | SEQUENCING | | | | |
| | | IN PROGRESS | | | | |
| | | *** from contig | | | | |
| | | 4-106, complete | | | | |
| | | sequence | | | | |
| 682 | U95094 | Xenopus laevis | 5.00E−04 | VSM2_TRYBB | VARIANT | 4.3 |
| | | XL-INCENP | | | SURFACE | |
| | | (XL-INCENP) | | | GLYCOPROTEIN | |
| | | mRNA, complete | | | MITAT 1.2 | |
| | | cds | | | PRECURSOR (VSG | |
| | | | | | 221) | |
| 683 | U00238 | Homo sapiens | 0 | <NONE> | <NONE> | <NONE> |
| | | glutamine PRPP | | | | |
| | | amidotransferase | | | | |
| | | (GPAT) mRNA, | | | | |
| | | complete cds | | | | |
| 684 | U95102 | Xenopus laevis | 0.005 | PRPR_SALTY | PROPIONATE | 1.5 |
| | | mitotic | | | CATABOLISM | |
| | | phosphoprotein | | | OPERON | |
| | | 90 mRNA, | | | REGULATORY | |
| | | complete cds | | | PROTEIN | |
| 685 | U95102 | Xenopus laevis | 7.00E−07 | YAND_SCHPO | HYPOTHETICAL | 0.38 |
| | | mitotic | | | 30.4 KD PROTEIN | |
| | | phosphoprotein | | | C3H1.13 IN | |
| | | 90 mRNA, | | | CHROMOSOME I | |
| | | complete cds | | | | |
| 686 | D25538 | Human mRNA | 0 | <NONE> | <NONE> | <NONE> |
| | | for KIAA0037 | | | | |
| | | gene, complete | | | | |
| | | cds | | | | |
| 687 | U95102 | Xenopus laevis | 2.00E−07 | A1AA_RAT | ALPHA-1A | 4.4 |
| | | mitotic | | | ADRENERGIC | |
| | | phosphoprotein | | | RECEPTOR (RA42) | |
| | | 90 mRNA, | | | | |
| | | complete cds | | | | |
| 688 | L26956 | Mesocricetus | 4.00E−33 | <NONE> | <NONE> | <NONE> |
| | | auratus stearyl- | | | | |
| | | CoA desaturase | | | | |
| | | sequence | | | | |
| | | including male | | | | |
| | | hormone | | | | |
| | | dependent gene | | | | |
| | | derived from | | | | |
| | | hamster | | | | |
| | | frankorgan | | | | |
| 689 | U95102 | Xenopus laevis | 3.00E−10 | <NONE> | <NONE> | <NONE> |
| | | mitotic | | | | |
| | | phosphoprotein | | | | |
| | | 90 mRNA, | | | | |
| | | complete cds | | | | |
| 690 | U95102 | Xenopus laevis | 3.00E−09 | YO93_CAEEL | HYPOTHETICAL | 2.00E−08 |
| | | mitotic | | | 58.5 KD PROTEIN | |
| | | phosphoprotein | | | T20B12.3 IN | |
| | | 90 mRNA, | | | CHROMOSOME III | |
| | | complete cds | | | | |
| 691 | U95102 | Xenopus laevis | 8.00E−09 | <NONE> | <NONE> | <NONE> |
| | | mitotic | | | | |
| | | phosphoprotein | | | | |
| | | 90 mRNA, | | | | |
| | | complete cds | | | | |
| 692 | AB017026 | Mus musculus | 0 | OXYB_RABIT | OXYSTEROL- | 1.00E−34 |
| | | mRNA for | | | BINDING PROTEIN | |
| | | oxysterol-binding | | | | |
| | | protein, complete | | | | |
| | | cds | | | | |
| 693 | U95098 | Xenopus laevis | 6.00E−04 | UFO2_MAIZE | FLAVONOL 3-O- | 3.1 |
| | | mitotic | | | GLUCOSYLTRANS | |
| | | phosphoprotein | | | FERASE (EC | |
| | | 44 mRNA, partial | | | 2.4.1.91) | |
| | | cds | | | | |
| 694 | U95102 | Xenopus laevis | 5.00E−04 | <NONE> | <NONE> | <NONE> |
| | | mitotic | | | | |
| | | phosphoprotein | | | | |
| | | 90 mRNA, | | | | |
| | | complete cds | | | | |
| 695 | U34954 | Caenorhabditis | 5.00E−24 | CYPA_CAEEL | PEPTIDYL-PROLYL | 2.00E−29 |
| | | elegans | | | CIS-TRANS | |
| | | cyclophilin | | | ISOMERASE 10 (EC | |
| | | isoform 10 | | | 5.2.1.8) | |
| 696 | AB011167 | Homo sapiens | 0 | RFX5_HUMAN | BINDING | 2.1 |
| | | mRNA for | | | REGULATORY | |
| | | KIAA0595 | | | FACTOR | |
| | | protein, partial | | | | |
| | | cds | | | | |
| 697 | U03886 | Human GS2 | 2.00E−28 | SKD1_MOUSE | SKD1 PROTEIN | 4.00E−17 |
| | | mRNA, complete | | | | |
| | | cds. | | | | |
| 698 | AF086275 | Homo sapiens full | 3.00E−41 | SPT7_YEAST | TRANSCRIPTIONA | 0.82 |
| | | length insert | | | L ACTIVATOR SPT7 | |
| | | cDNA clone | | | | |
| | | ZD45C02 | | | | |
| 699 | U95102 | Xenopus laevis | 3.00E−10 | CA1E_HUMAN | COLLAGEN ALPHA | 1.1 |
| | | mitotic | | | 1(XV) CHAIN | |
| | | phosphoprotein | | | PRECURSOR | |
| | | 90 mRNA, | | | | |
| | | complete cds | | | | |
| 700 | U95102 | Xenopus laevis | 4.00E−11 | E434_ADECC | Q65962 canine | 4.4 |
| | | mitotic | | | adenovirus type 1 | |
| | | phosphoprotein | | | (strain cll). early e4 31 | |
| | | 90 mRNA, | | | kd protein. 11/98 | |
| | | complete cds | | | | |
| 701 | L17340 | Drosophila | 3.3 | CISY_TETTH | CITRATE | 9.7 |
| | | melanogaster | | | SYNTHASE, | |
| | | germline | | | MITOCHONDRIAL | |
| | | transcription | | | PRECURSOR (EC | |
| | | factor gene, | | | 4.1.3.7) (14 NM | |
| | | complete cds. | | | FILAMENT- | |
| | | | | | FORMING | |
| | | | | | PROTEIN) | |
| 702 | X58170 | M. musculus | 2.00E−45 | PME2_LYCES | PECTINESTERASE | 7.4 |
| | | mRNA for t- | | | 2 PRECURSOR (EC | |
| | | Complex Tcp-10a | | | 3.1.1.11) (PECTIN | |
| | | gene | | | METHYLESTERASE | |
| | | | | | ) (PE 2) | |
| 703 | Z96207 | H. sapiens | 8.00E−08 | <NONE> | <NONE> | <NONE> |
| | | telomeric DNA | | | | |
| | | sequence, clone | | | | |
| | | 12PTEL049, read | | | | |
| | | 12PTELOO049.seq | | | | |
| 704 | X58430 | Human Hox1.8 | e−146 | HXAA_HUMAN | HOMEOBOX | 4.00E−05 |
| | | gene | | | PROTEIN HOX-A10 | |
| | | | | | (HOX-1H) (HOX-1.8) | |
| | | | | | (PL) | |
| 705 | U95094 | Xenopus laevis | 6.00E−06 | YN39_SYNP7 | HYPOTHETICAL 9.2 | 0.89 |
| | | XL-INCENP | | | KD PROTEIN IN | |
| | | (XL-INCENP) | | | CYST-CYSR | |
| | | mRNA, complete | | | INTERGENIC | |
| | | cds | | | REGION (ORF 81) | |
| 706 | U95094 | Xenopus laevis | 1.00E−11 | MYSH_BOVIN | MYOSIN I HEAVY | 0.001 |
| | | XL-INCENP | | | CHAIN-LIKE | |
| | | (XL-INCENP) | | | PROTEIN (MIHC) | |
| | | mRNA, complete | | | (BRUSH BORDER | |
| | | cds | | | MYOSIN I) (BBMI) | |
| 707 | M19961 | Human | e−123 | OTHU5B | <NONE> | 3.00E−30 |
| | | cytochrome c | | | | |
| | | oxidase subunit | | | | |
| | | Vb (coxVb) | | | | |
| | | mRNA, complete | | | | |
| | | cds. | | | | |
| 708 | X68380 | M. musculus gene | 5.00E−04 | 42_MOUSE | ERYTHROCYTE | 9.9 |
| | | for cathepsin D, | | | MEMBRANE | |
| | | exon 3 | | | PROTEIN BAND 4.2 | |
| | | | | | (P4.2) (PALLIDIN) | |
| 709 | U95102 | Xenopus laevis | 1.00E−11 | TCPA_DROME | T-COMPLEX | 4.3 |
| | | mitotic | | | PROTEIN 1, ALPHA | |
| | | phosphoprotein | | | SUBUNIT (TCP-1- | |
| | | 90 mRNA, | | | ALPHA) | |
| | | complete cds | | | | |
| 710 | U95102 | Xenopus laevis | 3.00E−10 | <NONE> | <NONE> | <NONE> |
| | | mitotic | | | | |
| | | phosphoprotein | | | | |
| | | 90 mRNA, | | | | |
| | | complete cds | | | | |
| 711 | U95094 | Xenopus laevis | 4.00E−12 | <NONE> | <NONE> | <NONE> |
| | | XL-INCENP | | | | |
| | | (XL-INCENP) | | | | |
| | | mRNA, complete | | | | |
| | | cds | | | | |
| 712 | U95102 | Xenopus laevis | 0.002 | <NONE> | <NONE> | <NONE> |
| | | mitotic | | | | |
| | | phosphoprotein | | | | |
| | | 90 mRNA, | | | | |
| | | complete cds | | | | |
| 713 | AB018323 | Homo sapiens | 3.00E−41 | LBR_CHICK | LAMIN B | 3.4 |
| | | mRNA for | | | RECEPTOR | |
| | | KIAA0780 | | | | |
| | | protein, partial | | | | |
| | | cds | | | | |
| 714 | U95102 | Xenopus laevis | 6.00E−06 | YM8L_YEAST | HYPOTHETICAL | 3.00E−08 |
| | | mitotic | | | 71.1 KD PROTEIN | |
| | | phosphoprotein | | | IN DSK2-CAT8 | |
| | | 90 mRNA, | | | INTERGENIC | |
| | | complete cds | | | REGION | |
| 715 | U95102 | Xenopus laevis | 4.00E−13 | PSC_DROME | POSTERIOR SEX | 0.6 |
| | | mitotic | | | COMBS PROTEIN | |
| | | phosphoprotein | | | | |
| | | 90 mRNA, | | | | |
| | | complete cds | | | | |
| 716 | L28101 | Homo sapiens | 7.00E−07 | IRKX_RAT | INWARD | 5.4 |
| | | kallistatin (PI4) | | | RECTIFIER | |
| | | gene, exons 1-4, | | | POTASSIUM | |
| | | complete cds | | | CHANNEL BIR9 | |
| | | | | | (KIR5.1) | |
| 717 | AC001038 | Homo sapiens | 8.00E−09 | MGMT_YEAST | METHYLATED- | 0.48 |
| | | (subclone 2_h2 | | | DNA- PROTEIN- | |
| | | from P1 H49) | | | CYSTEINE | |
| | | DNA sequence | | | METHYLTRANSFE | |
| | | | | | RASE | |
| 718 | U95094 | Xenopus laevis | 1.00E−11 | YWDE_BACSU | HYPOTHETICAL | 1.8 |
| | | XL-INCENP | | | 19.9 KD PROTEIN | |
| | | (XL-INCENP) | | | IN SACA-UNG | |
| | | mRNA, complete | | | INTERGENIC | |
| | | cds | | | REGION | |
| | | | | | PRECURSOR | |
| 719 | U01139 | Mus musculus | e−110 | GSC_DROME | HOMEOBOX | 7.2 |
| | | B6D2F1 clone | | | PROTEIN | |
| | | 2C11B mRNA. | | | GOOSECOID | |
| 720 | AB017430 | Homo sapiens | 0 | YBAV_ECOLI | HYPOTHETICAL | 0.17 |
| | | mRNA for | | | 12.7 KD PROTEIN | |
| | | kinesin-like DNA | | | IN HUPB-COF | |
| | | binding protein, | | | INTERGENIC | |
| | | complete cds | | | REGION | |
| 721 | U95094 | Xenopus laevis | 0.001 | CPCF_SYNP2 | PHYCOCYANOBILI | 2.4 |
| | | XL-INCENP | | | N LYASE BETA | |
| | | (XL-INCENP) | | | SUBUNIT (EC 4.-.-.-) | |
| | | mRNA, complete | | | | |
| | | cds | | | | |
| 722 | U95102 | Xenopus laevis | 9.00E−10 | <NONE> | <NONE> | <NONE> |
| | | mitotic | | | | |
| | | phosphoprotein | | | | |
| | | 90 mRNA, | | | | |
| | | complete cds | | | | |
| 723 | U95102 | Xenopus laevis | 0.04 | YKK7_CAEEL | HYPOTHETICAL | 0.057 |
| | | mitotic | | | 54.9 KD PROTEIN | |
| | | phosphoprotein | | | C02F5.7 IN | |
| | | 90 mRNA, | | | CHROMOSOME III | |
| | | complete cds | | | | |
| 724 | U95094 | Xenopus laevis | 8.00E−08 | H5_CAIMO | HISTONE H5 | 0.39 |
| | | XL-INCENP | | | | |
| | | (XL-INCENP) | | | | |
| | | mRNA, complete | | | | |
| | | cds | | | | |
| 725 | U95094 | Xenopus laevis | 3.00E−09 | DED1_YEAST | PUTATIVE ATP- | 0.5 |
| | | XL-INCENP | | | DEPENDENT RNA | |
| | | (XL-INCENP) | | | HELICASE DED1 | |
| | | mRNA, complete | | | | |
| | | cds | | | | |
| 726 | J04617 | Human elongation | 5.00E−36 | ALU7_HUMAN | !!!ALU | 0.84 |
| | | factor EF-1-alpha | | | SUBFAMILY SQ | |
| | | gene, complete | | | WARNING ENTRY | |
| | | cds.>:: | | | !!! | |
| | | dbj\|E02629\|E0262 | | | | |
| | | 9 DNA of human | | | | |
| | | polypeptide chain | | | | |
| | | elongation factor- | | | | |
| | | 1 alpha | | | | |
| 727 | X54859 | Porcine TNF- | 3.3 | Z165_HUMAN | ZINC FINGER | 5.6 |
| | | alpha and TNF- | | | PROTEIN 165 | |
| | | beta genes for | | | | |
| | | tumour necrosis | | | | |
| | | factors alpha and | | | | |
| | | beta, respectively. | | | | |
| 728 | D49911 | Thermus | 0.014 | CC48_CAPAN | CELL DIVISION | 9.9 |
| | | thermophilus | | | CYCLE PROTEIN 48 | |
| | | UvrA gene, | | | HOMOLOG | |
| | | complete cds | | | | |
| 729 | U95098 | Xenopus laevis | 2.00E−06 | CA25_HUMAN | PROCOLLAGEN | 0.011 |
| | | mitotic | | | ALPHA 2(V) CHAIN | |
| | | phosphoprotein | | | PRECURSOR | |
| | | 44 mRNA, partial | | | | |
| | | cds | | | | |
| 730 | D15057 | Human mRNA | 0 | DAD1_HUMAN | DEFENDER | 8.00E−16 |
| | | for DAD-1, | | | AGAINST CELL | |
| | | complete cds | | | DEATH 1 (DAD-1) | |
| 731 | U95098 | Xenopus laevis | 6.00E−06 | ANFD_RHOCA | NITROGENASE | 9.6 |
| | | mitotic | | | IRON-IRON | |
| | | phosphoprotein | | | PROTEIN ALPHA | |
| | | 44 mRNA, partial | | | CHAIN (EC 1.18.6.1) | |
| | | cds | | | (NITROGENASE | |
| | | | | | COMPONENT I) | |
| | | | | | (DINITROGENASE) | |
| 732 | U95098 | Xenopus laevis | 7.00E−07 | EFTU_CHLVI | ELONGATION | 2.5 |
| | | mitotic | | | FACTOR TU (EF- | |
| | | phosphoprotein | | | TU) | |
| | | 44 mRNA, partial | | | | |
| | | cds | | | | |
| 733 | AB018335 | Homo sapiens | 0 | TRYM_RAT | MAST CELL | 5.6 |
| | | mRNA for | | | TRYPTASE | |
| | | KIAA0792 | | | PRECURSOR (EC | |
| | | protein, complete | | | 3.4.21.59) | |
| | | cds | | | | |
| 734 | X98743 | H. sapiens mRNA | 0.04 | <NONE> | <NONE> | <NONE> |
| | | for RNA helicase | | | | |
| | | (Myc-regulated | | | | |
| | | dead box protein) | | | | |
| 735 | U95098 | Xenopus laevis | 2.00E−07 | <NONE> | <NONE> | <NONE> |
| | | mitotic | | | | |
| | | phosphoprotein | | | | |
| | | 44 mRNA, partial | | | | |
| | | cds | | | | |
| 736 | Z49314 | S. cerevisiae | 3.2 | <NONE> | <NONE > | <NONE> |
| | | chromosome X | | | | |
| | | reading frame | | | | |
| | | ORF YJL039c | | | | |
| 737 | D12646 | Mouse kif4 | 0 | KIF4_MOUSE | KINESIN-LIKE | 2.00E−76 |
| | | mRNA for | | | PROTEIN KIF4 | |
| | | microtubule− | | | | |
| | | based motor | | | | |
| | | protein KIF4, | | | | |
| | | complete cds | | | | |
| 738 | J04038 | Human | 2.00E−47 | SDC1_HUMAN | SYNDECAN-1 | 3.5 |
| | | glyceraldehyde−3- | | | PRECURSOR | |
| | | phosphate | | | (SYND1) (CD138) | |
| | | dehydrogenase | | | | |
| 739 | AF010238 | Homo sapiens | 1.00E−09 | LIN1_HUMAN | LINE−1 REVERSE | 0.001 |
| | | von Hippel- | | | TRANSCRIPTASE | |
| | | Lindau tumor | | | HOMOLOG | |
| | | suppressor | | | | |
| 740 | U95102 | Xenopus laevis | 2.00E−06 | YQJX_BACSU | HYPOTHETICAL | 9.9 |
| | | mitotic | | | 13.2 KD PROTEIN | |
| | | phosphoprotein | | | IN GLNQ-ANSR | |
| | | 90 mRNA, | | | INTERGENIC | |
| | | complete cds | | | REGION | |
| 741 | L21186 | Human lysyl | e−145 | OXRTL | <NONE> | 1.00E−34 |
| | | oxidase−like | | | | |
| | | protein mRNA, | | | | |
| | | complete cds. | | | | |
| 742 | U95094 | Xenopus laevis | 2.00E−05 | CC48_SOYBN | CELL DIVISION | 7.6 |
| | | XL-INCENP | | | CYCLE PROTEIN 48 | |
| | | (XL-INCENP) | | | HOMOLOG | |
| | | mRNA, complete | | | (VALOSIN | |
| | | cds | | | CONTAINING | |
| | | | | | PROTEIN | |
| | | | | | HOMOLOG) (VCP) | |
| 743 | AF009203 | Homo sapiens | 3.3 | <NONE> | <NONE> | <NONE> |
| | | YAC clone | | | | |
| | | 377A1 unknown | | | | |
| | | mRNA, | | | | |
| | | 3′ untranslated | | | | |
| | | region | | | | |
| 744 | Z74894 | S. cerevisiae | 0.12 | CD14_RABIT | Q28680 oryctolagus | 1.9 |
| | | chromosome XV | | | cuniculus (rabbit). | |
| | | reading frame | | | monocyte | |
| | | ORF YOL152w | | | differentiation antigen | |
| | | | | | cd14 precursor. 11/98 | |
| 745 | U95094 | Xenopus laevis | 9.00E−10 | KIN3_YEAST | SERINE/THREONIN | 2.5 |
| | | XL-INCENP | | | E−PROTEIN KINASE | |
| | | (XL-INCENP) | | | KIN3 (EC 2.7.1.-) | |
| | | mRNA, complete | | | | |
| | | cds | | | | |
| 746 | U95102 | Xenopus laevis | 2.00E−05 | YA53_SCHPO | HYPOTHETICAL | 7.00E−17 |
| | | mitotic | | | 24.2 KD PROTEIN | |
| | | phosphoprotein | | | C13A11.03 IN | |
| | | 90 mRNA, | | | CHROMOSOME I | |
| | | complete cds | | | | |
| 747 | S61044 | ALDH3'2 aldehyd | 0 | DHAP_HUMAN | ALDEHYDE | 2.00E−71 |
| | | e dehydrogenase | | | DEHYDROGENASE, | |
| | | isozyme 3 | | | DIMERIC NADP- | |
| | | [human, stomach, | | | PREFERRING (EC | |
| | | mRNA Partial, | | | 1.2.1.5) (CLASS 3) | |
| | | 1362 nt] | | | | |
| 748 | U95094 | Xenopus laevis | 2.00E−08 | CA1E_CHICK | COLLAGEN ALPHA | 0.36 |
| | | XL-INCENP | | | 1(XIV) CHAIN | |
| | | (XL-INCENP) | | | PRECURSOR | |
| | | mRNA, complete | | | (UNDULIN) | |
| | | cds | | | | |
| 749 | U95102 | Xenopus laevis | 7.00E−06 | <NONE> | <NONE> | <NONE> |
| | | mitotic | | | | |
| | | phosphoprotein | | | | |
| | | 90 mRNA, | | | | |
| | | complete cds | | | | |
| 750 | L14815 | Entamoeba | 0.12 | <NONE> | <NONE> | <NONE> |
| | | histolytica HM- | | | | |
| | | 1:IMSS galactose− | | | | |
| | | specific adhesin | | | | |
| | | 170 kD subunit | | | | |
| | | (hg13) gene, | | | | |
| | | complete cds. | | | | |
| 751 | X63785 | T. thermophila | 1.1 | <NONE> | <NONE> | <NONE> |
| | | gene for snRNA | | | | |
| | | U2-2 | | | | |
| 752 | M83756 | Mytilus edulis | 0.042 | DSC1_HUMAN | DESMOCOLLIN | 2.6 |
| | | mitochondrial | | | 1A/1B PRECURSOR | |
| | | NADH | | | (DESMOSOMAL | |
| | | dehydrogenase | | | GLYCOPROTEIN | |
| | | subunit 5 (ND5) | | | 2/3) (DG2 / DG3) | |
| | | gene, 3′ end; | | | | |
| | | NADH | | | | |
| | | dehydrogenase | | | | |
| | | subunit 6 (ND6) | | | | |
| | | gene, complete | | | | |
| | | cds; and | | | | |
| | | cytochrome b (cyt | | | | |
| | | b), 5′ end. | | | | |
| 753 | AB001066 | Brown trout | 0.38 | IMB3_HUMAN | IMPORTIN BETA-3 | 1.2 |
| | | microsatellite | | | SUBUNIT | |
| | | DNA sequence | | | (KARYOPHERIN | |
| | | | | | BETA-3 SUBUNIT) | |
| 754 | AF064787 | Lotus japonicus | 0.51 | <NONE> | <NONE> | <NONE> |
| | | rac GTPase | | | | |
| | | activating protein | | | | |
| | | 1 mRNA, | | | | |
| | | complete cds | | | | |
| 755 | U20608 | Dictyostelium | 0.043 | <NONE> | <NONE> | <NONE> |
| | | discoideum | | | | |
| | | unknown spore | | | | |
| | | germination- | | | | |
| | | specific protein- | | | | |
| | | like protein, orf1, | | | | |
| | | orf2 and orf3 | | | | |
| | | genes, complete | | | | |
| | | cds | | | | |
| 756 | M77812 | Rabbit myosin | 1.2 | RBL1_HUMAN | RETINOBLASTOM | 4.9 |
| | | heavy chain | | | A-LIKE PROTEIN 1 | |
| | | mRNA, complete | | | (107 KD | |
| | | cds. | | | RETINOBLASTOM | |
| | | | | | A-ASSOCIATED | |
| | | | | | PROTEIN) (PRB1) | |
| | | | | | (P107) | |
| 757 | X63789 | T. thermophila | 0.058 | <NONE> | <NONE> | <NONE> |
| | | genes for snRNA | | | | |
| | | U5-1, snRNA U5- | | | | |
| | | 2 | | | | |
| 758 | D50646 | Mouse mRNA for | 2.00E−27 | PMT3_YEAST | DOLICHYL- | 0.002 |
| | | SDF2, complete | | | PHOSPHATE- | |
| | | cds | | | MANNOSE− | |
| | | | | | PROTEIN | |
| | | | | | MANNOSYLTRANS | |
| | | | | | FERASE 3 (EC | |
| | | | | | 2.4.1.109) | |
| 759 | L81583 | Homo sapiens | 3.00E−19 | ALU5_HUMAN | !!!! ALU | 0.86 |
| | | (subclone 3_g2 | | | SUBFAMILY SC | |
| | | from P1 H11) | | | WARNING ENTRY | |
| | | DNA sequence | | | !!!! | |
| 760 | U95102 | Xenopus laevis | 2.00E−06 | SYFA_YEAST | PHENYLALANYL- | 5.7 |
| | | mitotic | | | TRNA | |
| | | phosphoprotein | | | SYNTHETASE | |
| | | 90 mRNA, | | | ALPHA CHAIN | |
| | | complete cds | | | CYTOPLASMIC | |
| 761 | AF000370 | Homo sapiens | 6.00E−89 | APP1_MOUSE | AMYLOID-LIKE | 5.7 |
| | | polymorphic CA | | | PROTEIN 1 | |
| | | dinucleotide | | | PRECURSOR | |
| | | repeat flanking | | | (APLP) | |
| | | region | | | | |
| 762 | U95098 | Xenopus laevis | 0.002 | <NONE> | <NONE> | <NONE> |
| | | mitotic | | | | |
| | | phosphoprotein | | | | |
| | | 44 mRNA, partial | | | | |
| | | cds | | | | |
| 763 | U95102 | Xenopus laevis | 7.00E−06 | PSF_HUMAN | PTB-ASSOCIATED | 0.72 |
| | | mitotic | | | SPLICING FACTOR | |
| | | phosphoprotein | | | (PSF) | |
| | | 90 mRNA, | | | | |
| | | complete cds | | | | |
| 764 | AB018288 | Homo sapiens | 0 | TC2A_CAEBR | TRANSPOSABLE | 1.5 |
| | | mRNA for | | | ELEMENT TCB2 | |
| | | KIAA0745 | | | TRANSPOSASE | |
| | | protein, partial | | | | |
| | | cds | | | | |
| 765 | AF020282 | Dictyostelium | 0.38 | PMT2_YEAST | DOLICHYL- | 0.18 |
| | | discoideum | | | PHOSPHATE− | |
| | | DG2033 gene, | | | MANNOSE− | |
| | | partial cds | | | PROTEIN | |
| | | | | | MANNOSYLTRANS | |
| | | | | | FERASE 2 (EC | |
| | | | | | 2.4.1.109) | |
| 766 | AF017357 | Oryza sativa low | 0.38 | RGS3_HUMAN | REGULATOR OF G- | 0.23 |
| | | molecular early | | | PROTEIN | |
| | | light-inducible | | | SIGNALLING 3 | |
| | | protein mRNA, | | | (RGS3) (RGP3) | |
| | | complete cds | | | | |
| 767 | U67599 | Methanococcus | 0.13 | <NONE> | <NONE> | <NONE> |
| | | jannaschii section | | | | |
| | | 141 of 150 of the | | | | |
| | | complete genome | | | | |
| 768 | X74178 | B. taurus | 0.13 | FAG1_SYNY3 | P73574 synechocystis | 5.00E−16 |
| | | microsatellite | | | sp. (strain pcc 6803). | |
| | | DNA INRA153 | | | 3-oxoacyl-[acyl- | |
| | | | | | carrier protein] | |
| | | | | | reductase 1 (ec | |
| | | | | | 1.1.1.100) (3- | |
| | | | | | ketoacyl-acyl carrier | |
| | | | | | protein reductase 1). | |
| | | | | | 11/98 | |
| 769 | AF041858 | Mus musculus | 0.043 | CA44_HUMAN | COLLAGEN ALPHA | 0.24 |
| | | synaptojanin 2 | | | 4(IV) CHAIN | |
| | | isoform delta | | | PRECURSOR | |
| | | mRNA, partial | | | | |
| | | cds | | | | |
| 770 | J01404 | Drosophila | 0.021 | NU1M_CITLA | NADH- | 7.2 |
| | | melanogaster | | | UBIQUINONE | |
| | | mitochondrial | | | OXIDOREDUCTASE | |
| | | cytochrome c | | | CHAIN 1 (EC 1.6.5.3) | |
| | | oxidase subunits, | | | | |
| | | ATPase6, 7 | | | | |
| | | tRNAs (Trp, Cys, | | | | |
| | | Tyr, Leu(UUR), | | | | |
| | | Lys, Asp, Gly) | | | | |
| | | genes, and | | | | |
| | | unidentified | | | | |
| | | reading frames | | | | |
| | | A61, 2 and 3. | | | | |
| 771 | AL022317 | Human DNA | 3.00E−41 | ALU7_HUMAN | !!!! ALU | 4.00E−08 |
| | | sequence from | | | SUBFAMILY SQ | |
| | | clone 140L1 on | | | WARNING ENTRY | |
| | | chromosome | | | !!!! | |
| | | 22q13.1-13.31, | | | | |
| | | complete | | | | |
| | | sequence [Homo | | | | |
| | | sapiens ] | | | | |
| 772 | U95094 | Xenopus laevis | 1.00E−09 | <NONE> | <NONE> | <NONE> |
| | | XL-INCENP | | | | |
| | | (XL-INCENP) | | | | |
| | | mRNA, complete | | | | |
| | | cds | | | | |
| 773 | AF095927 | Rattus norvegicus | 0 | P2C_PARTE | PROTEIN | 1.00E−16 |
| | | protein | | | PHOSPHATASE 2C | |
| | | phosphatase 2C | | | (EC 3.1.3.16) (PP2C) | |
| | | mRNA, complete | | | | |
| | | cds | | | | |
| 774 | X87212 | H. sapiens mRNA | 0 | CATC_HUMAN | DIPEPTIDYL- | 2.00E−46 |
| | | for cathepsin C | | | PEPTIDASE I | |
| | | | | | PRECURSOR (EC | |
| | | | | | 3.4.14.1) | |
| 775 | X05283 | Drosophila | 4.5 | <NONE> | <NONE> | <NONE> |
| | | melanogaster | | | | |
| | | PKCG7 gene | | | | |
| | | exons 7-14 for | | | | |
| | | protein kinase C | | | | |
| 776 | X03558 | Human mRNA | 0 | EF11_HUMAN | ELONGATION | 1.00E−83 |
| | | for elongation | | | FACTOR 1-ALPHA 1 | |
| | | factor 1 alpha | | | (EF-1-ALPHA-1) | |
| | | subunit | | | | |
| 777 | X06960 | Aspergillus | 0.23 | <NONE> | <NONE> | <NONE> |
| | | nidulans | | | | |
| | | mitochondrial | | | | |
| | | DNA for | | | | |
| | | cytochrome | | | | |
| | | oxidase subunit 3, | | | | |
| | | tRNA-Tyr | | | | |
| 778 | U95102 | Xenopus laevis | 3.00E−09 | YMT8_YEAST | HYPOTHETICAL | 5.00E−07 |
| | | mitotic | | | 36.4 KD PROTEIN | |
| | | phosphoprotein | | | IN NUP116-FAR3 | |
| | | 90 mRNA, | | | INTERGENIC | |
| | | complete cds | | | REGION | |
| 779 | U95102 | Xenopus laevis | 2.00E−07 | NAT1_YEAST | N-TERMINAL | 5.00E−23 |
| | | mitotic | | | ACETYLTRANSFER | |
| | | phosphoprotein | | | ASE 1 (EC 2.3.1.88) | |
| | | 90 mRNA, | | | | |
| | | complete cds | | | | |
| 780 | U59706 | Gallus gallus | 0.014 | PPOL_SARPE | POLY (ADP- | 0.021 |
| | | alternatively | | | RIBOSE) | |
| | | spliced AMPA | | | POLYMERASE (EC | |
| | | glutamate | | | 2.4.2.30) (PARP) | |
| | | receptor, isoform | | | | |
| | | GluR2 flop, | | | | |
| | | (GluR2) mRNA, | | | | |
| | | partial cds. | | | | |
| 781 | U57391 | Rattus norvegicus | 1.00E−84 | <NONE> | <NONE> | <NONE> |
| | | FceRI gamma- | | | | |
| | | chain interacting | | | | |
| | | protein SH2-B | | | | |
| | | (SH2-B) mRNA, | | | | |
| | | complete cds | | | | |
| 782 | AB014591 | Homo sapiens | 7.00E−57 | SSGP_VOLCA | SULFATED | 5.3 |
| | | mRNA for | | | SURFACE | |
| | | KIAA0691 | | | GLYCOPROTEIN | |
| | | protein, complete | | | 185 (SSG 185) | |
| | | cds | | | | |
| 783 | AJ008065 | Chrysolina bankii | 0.043 | <NONE> | <NONE> | <NONE> |
| | | 16S rRNA gene, | | | | |
| | | mitotype B2 | | | | |
| 784 | AF067212 | Caenorhabditis | 0.005 | MEK1_RAT | MAPK/ERK KINASE | 4.5 |
| | | elegans cosmid | | | KINASE 1 (EC 2.7.1.- | |
| | | F37F2 | | | ) (MEK KINASE 1) | |
| 785 | U95094 | Xenopus laevis | 0.042 | <NONE> | <NONE> | <NONE> |
| | | XL-INCENP | | | | |
| | | (XL-INCENP) | | | | |
| | | mRNA, complete | | | | |
| | | cds | | | | |
| 786 | U95102 | Xenopus laevis | 9.00E−09 | <NONE> | <NONE> | <NONE> |
| | | mitotic | | | | |
| | | phosphoprotein | | | | |
| | | 90 mRNA, | | | | |
| | | complete cds | | | | |
| 787 | Y13401 | Homo sapiens | 8.00E−08 | <NONE> | <NONE> | <NONE> |
| | | CD3 delta gene, | | | | |
| | | enhancer | | | | |
| | | sequence | | | | |
| 788 | AE001038 | Archaeoglobus | 0.13 | <NONE> | <NONE> | <NONE> |
| | | fulgidus section | | | | |
| | | 69 of 172 of the | | | | |
| | | complete genome | | | | |
| 789 | U95102 | Xenopus laevis | 2.00E−06 | <NONE> | <NONE> | <NONE> |
| | | mitotic | | | | |
| | | phosphoprotein | | | | |
| | | 90 mRNA, | | | | |
| | | complete cds | | | | |
| 790 | AF041463 | Manihot esculenta | 1.4 | <NONE> | <NONE> | <NONE> |
| | | elongation factor | | | | |
| | | 1-alpha | | | | |
| 791 | U95102 | Xenopus laevis | 0.002 | HXA3_HAEIN | HEME:HEMOPEXIN | 2.7 |
| | | mitotic | | | -BINDING PROTEIN | |
| | | phosphoprotein | | | PRECURSOR | |
| | | 90 mRNA, | | | | |
| | | complete cds | | | | |
| 792 | Z12112 | pWE15A cosmid | 3.00E−29 | PKWA_THECU | PUTATIVE | 2.00E−04 |
| | | vector DNA | | | SERINE/THREONIN | |
| | | | | | E−PROTEIN KINASE | |
| | | | | | PKWA (EC 2.7.1.-) | |
| 793 | U85193 | Human nuclear | 4.00E−44 | <NONE> | <NONE> | <NONE> |
| | | factor I-B2 | | | | |
| | | (NFIB2) mRNA, | | | | |
| | | complete cds | | | | |
| 794 | U89331 | Human | 7.00E−06 | NRL_HUMAN | NEURAL RETINA- | 6.3 |
| | | pseudoautosomal | | | SPECIFIC LEUCINE | |
| | | homeodomain- | | | ZIPPER PROTEIN | |
| | | containing protein | | | (NRL) | |
| | | (PHOG) mRNA, | | | | |
| | | complete cds | | | | |
| 795 | AF055666 | Mus musculus | 0.52 | PSPD_BOVIN | PULMONARY | 0.33 |
| | | kinesin light chain | | | SURFACTANT- | |
| | | 2 (Klc2) mRNA, | | | ASSOCIATED | |
| | | complete cds | | | PROTEIN D | |
| | | | | | PRECURSOR | |
| 796 | L13321 | Homo sapiens | 0.14 | YRP2_YEAST | HYPOTHETICAL | 0.27 |
| | | iduronate−2- | | | 84.4 KD PROTEIN | |
| | | sulfatase (IDS) | | | IN RPC2/RET1 | |
| | | gene, exon 1, | | | 3′ REGION | |
| | | incomplete 5′ end. | | | | |
| 797 | AL010270 | Plasmodium | 0.37 | YTH3_CAEEL | HYPOTHETICAL | 2 |
| | | falciparum DNA | | | 75.5 KD PROTEIN | |
| | | *** | | | C14A4.3 IN | |
| | | SEQUENCING | | | CHROMOSOME II | |
| | | IN PROGRESS | | | | |
| | | *** from contig | | | | |
| | | 4-96, complete | | | | |
| | | sequence | | | | |
| 798 | U95098 | Xenopus laevis | 0.015 | IMB3_HUMAN | IMPORTIN BETA-3 | 0.063 |
| | | mitotic | | | SUBUNIT | |
| | | phosphoprotein | | | (KARYOPHERIN | |
| | | 44 mRNA, partial | | | BETA-3 SUBUNIT) | |
| | | cds | | | | |
| 799 | U70139 | Mus musculus | 0 | CCR4_YEAST | GLUCOSE− | 5.00E−11 |
| | | putative CCR4 | | | REPRESSIBLE | |
| | | protein mRNA, | | | ALCOHOL | |
| | | partial cds | | | DEHYDROGENASE | |
| | | | | | TRANSCRIPTIONA | |
| | | | | | L EFFECTOR | |
| | | | | | (CARBON | |
| | | | | | CATABOLITE | |
| | | | | | REPRESSOR | |
| | | | | | PROTEIN 4) | |
| 800 | L26507 | Mouse myocyte | 3.00E−41 | MNF_MOUSE | MYOCYTE | 4.00E−18 |
| | | nuclear factor | | | NUCLEAR FACTOR | |
| | | (MNF) mRNA, | | | (MNF) | |
| | | complete cds. | | | | |
| 801 | U20527 | Mus musculus | 0 | GRO_MOUSE | GROWTH REGULATED | 1.00E−28 |
| | | chemokine KC | | | PROTEIN PRECURSOR | |
| | | gene, 5′ region. | | | (PLATELET-DERIVED | |
| | | | | | GROWTH FACTOR- | |
| | | | | | INDUCIBLE PROTEIN | |
| | | | | | KC) (SECRETORY | |
| | | | | | PROTEIN N51) | |
| 802 | AF065482 | Homo sapiens | 0 | MYSA_DROME | MYOSIN HEAVY | 0.089 |
| | | sorting nexin 2 | | | CHAIN, MUSCLE | |
| | | (SNX2) mRNA, | | | | |
| | | complete cds | | | | |
| 803 | U05823 | Mus musculus | 1.00E−94 | M84D_DROME | MALE SPECIFIC SPERM | 0.099 |
| | | pericentrin mRNA, | | | PROTEIN MST84DD | |
| | | complete cds. | | | | |
| 804 | U67468 | Methanococcus | 0.4 | <NONE> | <NONE> | <NONE> |
| | | jannaschii section | | | | |
| | | 10 of 150 of the | | | | |
| | | complete genome | | | | |
| 805 | U14178 | Human type II IL-1 | 1.00E−19 | AMPH_HUMAN | AMPHIPHYSIN | 2.9 |
| | | receptor gene, exon | | | | |
| | | 1B | | | | |
| 806 | L40411 | Homo sapiens | 0 | TRI8_HUMAN | THYROID RECEPTOR | 4.00E−86 |
| | | thyroid receptor | | | INTERACTING PROTEIN | |
| | | interactor | | | 8 (TRIP8) | |
| 807 | D17218 | Human HepG2 3′ | e−136 | CA1A_HUMAN | COLLAGEN ALPHA 1(X) | 3.00E−04 |
| | | region MboI cDNA, | | | CHAIN PRECURSOR | |
| | | clone hmd3g02m3 | | | | |
| 808 | Z57610 | H. sapiens CpG | e−102 | HN3B_MOUSE | HEPATOCYTE | 1.00E−24 |
| | | DNA, clone 187a10, | | | NUCLEAR FACTOR 3- | |
| | | reverse read | | | BETA (HNF-3B) | |
| | | cpg187a10.rt1a. | | | | |
| 809 | D14678 | Human mRNA for | 0 | NCD_DROME | CLARET | 1.00E−70 |
| | | kinesin-related | | | SEGREGATIONAL | |
| | | protein, partial cds | | | PROTEIN | |
| 810 | X56317 | Xiphophorus | 0.49 | WN1B_MOUSE | WNT-10B PROTEIN | 7.2 |
| | | maculatus | | | PRECURSOR (WNT-12) | |
| | | Xmrk(proto- | | | | |
| | | oncogene) gene for | | | | |
| | | receptor tyrosine | | | | |
| | | kinase. | | | | |
| 811 | M36200 | Human | 0.2 | VE2_HPV14 | REGULATORY PROTEIN | 3.1 |
| | | synaptobrevin 1 | | | E2 | |
| | | (SYB1) gene, exon | | | | |
| | | 5. | | | | |
| 812 | M18157 | Human glandular | 1.5 | EKLF_MOUSE | ERYTHROID | 1.1 |
| | | kallikrein gene, | | | KRUEPPEL-LIKE | |
| | | complete cds. | | | TRANSCRIPTION | |
| | | | | | FACTOR (EKLF) | |
| 813 | D25215 | Human mRNA for | 1.9 | YXIS_SACER | HYPOTHETICAL 28.9 | 1.3 |
| | | KIAA0032 gene, | | | KD PROTEIN IN XIS | |
| | | complete cds | | | 5′ REGION (ORF1) | |
| 814 | M96628 | Human gene | 2.00E−06 | AGRI_DISOM | AGRIN (FRAGMENT) | 9.5 |
| | | sequence, 5′ end. | | | | |
| 815 | Z57610 | H. sapiens CpG | e−102 | HN3B_MOUSE | HEPATOCYTE | 1.00E−19 |
| | | DNA, clone 187a10, | | | NUCLEAR FACTOR 3- | |
| | | reverse read | | | BETA (HNF-3B) | |
| | | cpg187a10.rt1a. | | | | |
| 816 | X14168 | Human pLC46 with | 5.00E−16 | ZN44_HUMAN | ZINC FINGER PROTEIN | 1.6 |
| | | DNA replication | | | 44 (ZINC FINGER | |
| | | origin | | | PROTEIN KOX7) | |
| 817 | M19262 | Rat clathrin light | 0.28 | LMA_DROME | LAMININ ALPHA | 4.7 |
| | | chain (LCB3) | | | CHAIN PRECURSOR | |
| | | mRNA, complete | | | | |
| | | cds. | | | | |
| 818 | AF058055 | Mus musculus | 0.2 | <NONE> | <NONE> | <NONE> |
| | | monocarboxylate | | | | |
| | | transporter 1 | | | | |
| 819 | AB014570 | Homo sapiens | 0.16 | YGR1_YEAST | HYPOTHETICAL 34.8 | 4.00E−06 |
| | | mRNA for | | | KD PROTEIN IN SUT1- | |
| | | KIAA0670 protein, | | | RCK1 INTERGENIC | |
| | | partial cds | | | REGION | |
| 820 | M19262 | Rat clathrin light | 0.27 | LMA_DROME | LAMININ ALPHA | 4.5 |
| | | chain (LCB3) | | | CHAIN PRECURSOR | |
| | | mRNA, complete | | | | |
| | | cds. | | | | |
| 821 | Z54367 | H. sapiens gene for | 0.29 | YO93_CAEEL | HYPOTHETICAL 58.5 | 1.00E−14 |
| | | plectin | | | KD PROTEIN T20B12.3 | |
| | | | | | IN CHROMOSOME III | |
| 822 | AB017026 | Mus musculus | 0 | OXYB_HUMAN | OXYSTEROL-BINDING | 2.00E−49 |
| | | mRNA for | | | PROTEIN | |
| | | oxysterol-binding | | | | |
| | | protein, complete | | | | |
| | | cds | | | | |
| 823 | X58170 | M. musculus mRNA | 1.00E−20 | UL52_HSV11 | DNA | 5.3 |
| | | for t-Complex Tcp- | | | HELICASE/PRIMASE | |
| | | 10a gene | | | COMPLEX PROTEIN | |
| | | | | | (DNA REPLICATION | |
| | | | | | PROTEIN UL52) | |
| 824 | X58430 | Human Hox1.8 | 0 | HXAA_HUMAN | HOMEOBOX PROTEIN | 1.00E−44 |
| | | gene | | | HOX-A10 (HOX-1H) | |
| | | | | | (HOX-1.8) (PL) | |
| 825 | X53754 | Porcine | 1.3 | <NONE> | <NONE> | <NONE> |
| | | sarcoplasmic/endopl | | | | |
| | | asmic-reticulum | | | | |
| | | Ca(2+) pump gene 2 | | | | |
| | | 3′ -end region | | | | |
| 826 | AB005786 | Arabidopsis thaliana | 0.46 | <NONE> | <NONE> | <NONE> |
| | | tRNA-Glu gene | | | | |
| 827 | AB012130 | Homo sapiens | 1.9 | <NONE> | <NONE> | <NONE> |
| | | SBC2 mRNA for | | | | |
| | | sodium bicarbonate | | | | |
| | | cotransporter2, | | | | |
| | | complete cds | | | | |
| 828 | AB017430 | Homo sapiens | 0 | YBAV_ECOLI | HYPOTHETICAL 12.7 | 0.063 |
| | | mRNA for kinesin- | | | KD PROTEIN IN HUPB- | |
| | | like DNA binding | | | COF INTERGENIC | |
| | | protein, complete | | | REGION | |
| | | cds | | | | |
| 829 | AB007886 | Homo sapiens | 0.042 | YDF3_SCHPO | PROBABLE | 0.52 |
| | | KIAA0426 mRNA, | | | EUKARYOTIC | |
| | | complete cds | | | INITIATION FACTOR | |
| | | | | | C17C9.03 | |
| 830 | AB018335 | Homo sapiens | e−172 | UROT_BOVIN | TISSUE PLASMINOGEN | 0.86 |
| | | mRNA for | | | ACTIVATOR | |
| | | KIAA0792 protein, | | | PRECURSOR (EC | |
| | | complete cds | | | 3.4.21.68) | |
| 831 | D12646 | Mouse kif4 mRNA | 0 | KIF4_MOUSE | KINESIN-LIKE PROTEIN | 9.00E−96 |
| | | for microtubule− | | | KIF4 | |
| | | based motor protein | | | | |
| | | KIF4, complete cds | | | | |
| 832 | U38376 | Rattus norvegicus | 0.048 | <NONE> | <NONE> | <NONE> |
| | | cytosolic | | | | |
| | | phospholipase A2 | | | | |
| | | mRNA, complete | | | | |
| | | cds | | | | |
| 833 | L40411 | Homo sapiens | 0 | TRI8_HUMAN | THYROID RECEPTOR | 4.00E−86 |
| | | thyroid receptor | | | INTERACTING PROTEIN | |
| | | interactor | | | 8 (TRIP8) | |
| 834 | U08110 | Mus musculus | 8.00E−04 | YNW7_YEAST | HYPOTHETICAL 68.8 | 0.02 |
| | | RNA1 homolog | | | KD PROTEIN IN URE2- | |
| | | (Fug1) mRNA, | | | SSU72 INTERGENIC | |
| | | complete cds. | | | REGION | |
| 835 | D50646 | Mouse mRNA for | 1.00E−40 | YB64_YEAST | HYPOTHETICAL 57.2 | 4.9 |
| | | SDF2, complete cds | | | KD PROTEIN IN MET8- | |
| | | | | | HPC2 INTERGENIC | |
| | | | | | REGION | |
| 836 | D50646 | Mouse mRNA for | 1.00E−40 | YB64_YEAST | HYPOTHETICAL 57.2 | 4.9 |
| | | SDF2, complete cds | | | KD PROTEIN IN MET8- | |
| | | | | | HPC2 INTERGENIC | |
| | | | | | REGION | |
| 837 | U67459 | Methanococcus | 5.00E−05 | GCS1_HUMAN | MANNOSYL- | 9.2 |
| | | jannaschii section 1 | | | OLIGOSACCHARIDE | |
| | | of 150 of the | | | GLUCOSIDASE (EC | |
| | | complete genome | | | 3.2.1.106) | |
| 838 | U18657 | Haemophilus | 0.01 | STE6_YEAST | MATING FACTOR A | 7 |
| | | influenzae LeuA | | | SECRETION PROTEIN | |
| | | (leuA) gene, partial | | | STE6 (MULTIPLE DRUG | |
| | | cds, DprA (dprA+), | | | RESISTANCE PROTEIN | |
| | | orf272 and orf193 | | | HOMOLOG) (P- | |
| | | genes, complete cds, | | | GLYCOPROTEIN) | |
| | | and PfkA (pfkA) | | | | |
| | | gene, partial cds. | | | | |
| 839 | U12523 | Rattus norvegicus | 1.00E−10 | YMT8_YEAST | HYPOTHETICAL 36.4 | 2.00E−06 |
| | | ultraviolet B | | | KD PROTEIN IN | |
| | | radiation-activated | | | NUP116-FAR3 | |
| | | UV98 mRNA, | | | INTERGENIC REGION | |
| | | partial sequence. | | | | |
| 840 | D78255 | Mouse mRNA for | e−175 | <NONE> | <NONE> | <NONE> |
| | | PAP-1, complete | | | | |
| | | cds | | | | |
| 841 | D17263 | Human HepG2 3′ | 1.00E−58 | <NONE> | <NONE> | <NONE> |
| | | region MboI cDNA, | | | | |
| | | clone hmd5f07m3 | | | | |
| 842 | AF006751 | Homo sapiens | 0.061 | YRP2_YEAST | HYPOTHETICAL 84.4 | 2.00E−07 |
| | | ES/130 mRNA, | | | KD PROTEIN IN | |
| | | complete cds | | | RPC2/RET1 3′ REGION | |
| 843 | U67459 | Methanococcus | 6.00E−05 | YC14_METJA | HYPOTHETICAL | 8.1 |
| | | jannaschii section 1 | | | PROTEIN MJ1214 | |
| | | of 150 of the | | | | |
| | | complete genome | | | | |
| 844 | D88689 | Mus musculus | 0.084 | ICP0_HSV2H | TRANS-ACTING | 0.014 |
| | | mRNA for flt-1, | | | TRANSCRIPTIONAL | |
| | | complete cds | | | PROTEIN ICP0 (VMW118 | |
| | | | | | PROTEIN) | |

TABLE 5
All Differential Data for Libs 1-4 and 8-9

| | Cluster | Clones in | Clones in | Clones in | Clones in | Clones in | Clones in |
|---|---|---|---|---|---|---|---|
| Clone Name | ID | Lib1 | Lib2 | Lib3 | Lib4 | Lib8 | Lib9 |
| M00001340B:A06 | 17062 | 3 | 0 | 0 | 0 | 0 | 0 |
| M00001340D:F10 | 11589 | 2 | 2 | 1 | 3 | 3 | 8 |
| M00001341A:E12 | 4443 | 10 | 6 | 2 | 6 | 3 | 11 |
| M00001342B:E06 | 39805 | 2 | 0 | 0 | 0 | 1 | 0 |
| M00001343C:F10 | 2790 | 7 | 15 | 13 | 14 | 6 | 0 |
| M00001343D:H07 | 23255 | 3 | 0 | 1 | 1 | 0 | 0 |
| M00001345A:E01 | 6420 | 8 | 0 | 2 | 0 | 1 | 0 |
| M00001346A:F09 | 5007 | 4 | 8 | 3 | 6 | 2 | 6 |
| M00001346D:E03 | 6806 | 5 | 2 | 1 | 2 | 0 | 3 |
| M00001346D:G06 | 5779 | 5 | 4 | 3 | 4 | 0 | 0 |
| M00001346D:G06 | 5779 | 5 | 4 | 3 | 4 | 0 | 0 |
| M00001347A:B10 | 13576 | 5 | 0 | 0 | 0 | 12 | 11 |
| M00001348B:B04 | 16927 | 4 | 0 | 0 | 2 | 0 | 0 |
| M00001348B:G06 | 16985 | 4 | 0 | 0 | 0 | 0 | 0 |
| M00001349B:B08 | 3584 | 5 | 11 | 5 | 0 | 0 | 2 |
| M00001350A:H01 | 7187 | 5 | 3 | 1 | 0 | 1 | 0 |
| M00001351B:A08 | 3162 | 10 | 14 | 1 | 6 | 6 | 5 |
| M00001351B:A08 | 3162 | 10 | 14 | 1 | 6 | 6 | 5 |
| M00001352A:E02 | 16245 | 4 | 0 | 0 | 0 | 0 | 0 |
| M00001353A:G12 | 8078 | 4 | 3 | 1 | 0 | 1 | 0 |
| M00001353D:D10 | 14929 | 4 | 0 | 0 | 1 | 23 | 16 |
| M00001355B:G10 | 14391 | 3 | 1 | 0 | 0 | 0 | 0 |
| M00001357D:D11 | 4059 | 8 | 6 | 8 | 16 | 0 | 1 |
| M00001361A:A05 | 4141 | 5 | 2 | 10 | 16 | 4 | 27 |
| M00001361D:F08 | 2379 | 26 | 13 | 4 | 2 | 2 | 3 |
| M00001362B:D10 | 5622 | 7 | 4 | 2 | 13 | 1 | 2 |
| M00001362C:H11 | 945 | 9 | 21 | 2 | 1 | 0 | 0 |
| M00001365C:C10 | 40132 | 2 | 0 | 0 | 0 | 3 | 0 |
| M00001370A:C09 | 6867 | 7 | 3 | 0 | 0 | 0 | 0 |
| M00001371C:E09 | 7172 | 3 | 5 | 1 | 2 | 0 | 1 |
| M00001376B:G06 | 17732 | 1 | 3 | 5 | 0 | 1 | 4 |
| M00001378B:B02 | 39833 | 2 | 0 | 0 | 0 | 0 | 0 |
| M00001379A:A05 | 1334 | 27 | 38 | 35 | 28 | 3 | 0 |
| M00001380D:B09 | 39886 | 2 | 0 | 0 | 0 | 0 | 0 |
| M00001382C:A02 | 22979 | 2 | 1 | 0 | 0 | 0 | 0 |
| M00001383A:C03 | 39648 | 2 | 0 | 0 | 0 | 0 | 0 |
| M00001383A:C03 | 39648 | 2 | 0 | 0 | 0 | 0 | 0 |
| M00001386C:B12 | 5178 | 5 | 5 | 4 | 2 | 5 | 2 |
| M00001387A:C05 | 2464 | 5 | 19 | 25 | 16 | 1 | 0 |
| M00001387B:G03 | 7587 | 6 | 2 | 1 | 0 | 0 | 0 |
| M00001388D:G05 | 5832 | 10 | 3 | 0 | 1 | 5 | 0 |
| M00001389A:C08 | 16269 | 3 | 0 | 0 | 0 | 1 | 1 |
| M00001394A:F01 | 6583 | 2 | 7 | 3 | 2 | 0 | 0 |
| M00001395A:C03 | 4016 | 5 | 14 | 0 | 6 | 0 | 0 |
| M00001396A:C03 | 4009 | 6 | 4 | 13 | 5 | 4 | 10 |
| M00001402A:E08 | 39563 | 2 | 0 | 0 | 0 | 0 | 0 |
| M00001407B:D11 | 5556 | 8 | 1 | 5 | 0 | 2 | 0 |
| M00001409C:D12 | 9577 | 5 | 2 | 0 | 1 | 11 | 12 |
| M00001410A:D07 | 7005 | 8 | 2 | 0 | 0 | 0 | 0 |
| M00001412B:B10 | 8551 | 4 | 4 | 0 | 3 | 0 | 0 |
| M00001415A:H06 | 13538 | 5 | 0 | 0 | 0 | 9 | 1 |
| M00001416A:H01 | 7674 | 5 | 2 | 0 | 5 | 0 | 0 |
| M00001416B:H11 | 8847 | 4 | 1 | 3 | 0 | 6 | 1 |
| M00001417A:E02 | 36393 | 2 | 0 | 0 | 1 | 0 | 0 |
| M00001418B:F03 | 9952 | 4 | 2 | 1 | 1 | 0 | 0 |
| M00001418D:B06 | 8526 | 3 | 2 | 1 | 5 | 1 | 0 |
| M00001421C:F01 | 9577 | 5 | 2 | 0 | 1 | 11 | 12 |
| M00001423B:E07 | 15066 | 4 | 0 | 0 | 0 | 0 | 0 |
| M00001424B:G09 | 10470 | 5 | 1 | 0 | 2 | 0 | 1 |
| M00001425B:H08 | 22195 | 3 | 0 | 0 | 0 | 0 | 0 |
| M00001426D:C08 | 4261 | 4 | 9 | 7 | 9 | 12 | 15 |
| M00001428A:H10 | 84182 | 1 | 0 | 0 | 0 | 0 | 0 |
| M00001429A:H04 | 2797 | 15 | 11 | 18 | 16 | 1 | 14 |
| M00001429B:A11 | 4635 | 7 | 9 | 2 | 0 | 0 | 0 |
| M00001429D:D07 | 40392 | 2 | 0 | 1 | 8 | 12 | 16 |
| M00001439C:F08 | 40054 | 1 | 0 | 0 | 0 | 0 | 0 |
| M00001442C:D07 | 16731 | 3 | 1 | 0 | 0 | 0 | 0 |
| M00001445A:F05 | 13532 | 3 | 2 | 1 | 0 | 1 | 2 |
| M00001446A:F05 | 7801 | 5 | 2 | 4 | 6 | 1 | 0 |
| M00001447A:G03 | 10717 | 7 | 2 | 0 | 5 | 8 | 0 |
| M00001448D:C09 | 8 | 1850 | 2127 | 1703 | 3133 | 1355 | 122 |
| M00001448D:H01 | 36313 | 2 | 0 | 0 | 0 | 1 | 30 |
| M00001449A:A12 | 5857 | 6 | 2 | 3 | 4 | 0 | 0 |
| M00001449A:B12 | 41633 | 1 | 1 | 0 | 0 | 0 | 0 |
| M00001449A:D12 | 3681 | 12 | 5 | 10 | 1 | 2 | 5 |
| M00001449A:G10 | 36535 | 2 | 0 | 0 | 0 | 0 | 0 |
| M00001449C:D06 | 86110 | 1 | 0 | 0 | 0 | 0 | 0 |
| M00001450A:A02 | 39304 | 2 | 0 | 0 | 0 | 0 | 0 |
| M00001450A:A11 | 32663 | 1 | 1 | 0 | 0 | 0 | 0 |
| M00001450A:B12 | 82498 | 1 | 0 | 0 | 0 | 0 | 0 |
| M00001450A:D08 | 27250 | 2 | 0 | 0 | 0 | 0 | 0 |
| M00001452A:B04 | 84328 | 1 | 0 | 0 | 0 | 0 | 0 |
| M00001452A:B12 | 86859 | 1 | 0 | 0 | 0 | 0 | 0 |
| M00001452A:D08 | 1120 | 44 | 41 | 5 | 11 | 5 | 0 |
| M00001452A:F05 | 85064 | 1 | 0 | 0 | 0 | 0 | 0 |
| M00001452C:B06 | 16970 | 4 | 0 | 0 | 0 | 3 | 4 |
| M00001453A:E11 | 16130 | 3 | 1 | 0 | 0 | 0 | 1 |
| M00001453C:F06 | 16653 | 3 | 1 | 0 | 0 | 0 | 0 |
| M00001454A:A09 | 83103 | 1 | 0 | 0 | 0 | 0 | 0 |
| M00001454B:C12 | 7005 | 8 | 2 | 0 | 0 | 0 | 0 |
| M00001454D:G03 | 689 | 58 | 95 | 17 | 36 | 66 | 95 |
| M00001455A:E09 | 13238 | 4 | 1 | 0 | 0 | 0 | 0 |
| M00001455B:E12 | 13072 | 4 | 1 | 0 | 0 | 0 | 0 |
| M00001455D:F09 | 9283 | 4 | 1 | 0 | 1 | 0 | 1 |
| M00001455D:F09 | 9283 | 4 | 1 | 0 | 1 | 0 | 1 |
| M00001460A:F06 | 2448 | 23 | 22 | 2 | 3 | 3 | 1 |
| M00001460A:F12 | 39498 | 2 | 0 | 0 | 0 | 0 | 0 |
| M00001461A:D06 | 1531 | 20 | 23 | 32 | 17 | 14 | 14 |
| M00001463C:B11 | 19 | 1415 | 1203 | 1364 | 525 | 479 | 774 |
| M00001465A:B11 | 10145 | 2 | 0 | 2 | 0 | 0 | 0 |
| M00001466A:E07 | 4275 | 11 | 2 | 5 | 0 | 4 | 2 |
| M00001467A:B07 | 38759 | 2 | 0 | 0 | 0 | 1 | 1 |
| M00001467A:D04 | 39508 | 2 | 0 | 0 | 0 | 0 | 0 |
| M00001467A:D08 | 16283 | 3 | 0 | 0 | 0 | 0 | 0 |
| M00001467A:D08 | 16283 | 3 | 0 | 0 | 0 | 0 | 0 |
| M00001467A:E10 | 39442 | 2 | 0 | 0 | 0 | 0 | 0 |
| M00001468A:F05 | 7589 | 6 | 2 | 1 | 1 | 1 | 0 |
| M00001469A:C10 | 12081 | 4 | 0 | 0 | 0 | 0 | 0 |
| M00001469A:H12 | 19105 | 2 | 0 | 2 | 0 | 1 | 0 |
| M00001470A:B10 | 1037 | 53 | 48 | 4 | 22 | 0 | 0 |
| M00001470A:C04 | 39425 | 2 | 0 | 0 | 0 | 0 | 0 |
| M00001471A:B01 | 39478 | 2 | 0 | 0 | 0 | 0 | 0 |
| M00001481D:A05 | 7985 | 3 | 1 | 4 | 0 | 1 | 0 |
| M00001490B:C04 | 18699 | 2 | 1 | 0 | 0 | 0 | 3 |
| M00001494D:F06 | 7206 | 4 | 3 | 3 | 1 | 2 | 0 |
| M00001497A:G02 | 2623 | 12 | 4 | 31 | 4 | 6 | 1 |
| M00001499B:A11 | 10539 | 2 | 1 | 1 | 0 | 1 | 0 |
| M00001500A:C05 | 5336 | 9 | 2 | 4 | 8 | 3 | 15 |
| M00001500A:E11 | 2623 | 12 | 4 | 31 | 4 | 6 | 1 |
| M00001500C:E04 | 9443 | 4 | 2 | 1 | 1 | 0 | 0 |
| M00001501D:C02 | 9685 | 3 | 2 | 0 | 7 | 2 | 3 |
| M00001504C:A07 | 10185 | 5 | 1 | 0 | 0 | 2 | 4 |
| M00001504C:H06 | 6974 | 7 | 3 | 0 | 1 | 0 | 0 |
| M00001504D:G06 | 6420 | 8 | 0 | 2 | 0 | 1 | 0 |
| M00001507A:H05 | 39168 | 2 | 0 | 0 | 0 | 0 | 0 |
| M00001511A:H06 | 39412 | 2 | 0 | 0 | 0 | 0 | 0 |
| M00001512A:A09 | 39186 | 2 | 0 | 0 | 0 | 0 | 0 |
| M00001512D:G09 | 3956 | 9 | 9 | 5 | 2 | 0 | 0 |
| M00001513A:B06 | 4568 | 10 | 4 | 0 | 9 | 2 | 0 |
| M00001513C:E08 | 14364 | 1 | 0 | 0 | 0 | 0 | 0 |
| M00001514C:D11 | 40044 | 2 | 0 | 0 | 0 | 0 | 0 |
| M00001517A:B07 | 4313 | 13 | 6 | 1 | 0 | 1 | 0 |
| M00001518C:B11 | 8952 | 3 | 4 | 0 | 4 | 2 | 0 |
| M00001528A:C04 | 7337 | 4 | 4 | 3 | 16 | 12 | 21 |
| M00001528A:F09 | 18957 | 3 | 0 | 0 | 0 | 0 | 0 |
| M00001528B:H04 | 8358 | 3 | 3 | 2 | 0 | 0 | 0 |
| M00001531A:D01 | 38085 | 2 | 0 | 0 | 0 | 0 | 0 |
| M00001532B:A06 | 3990 | 6 | 12 | 4 | 1 | 3 | 1 |
| M00001533A:C11 | 2428 | 14 | 14 | 13 | 9 | 2 | 19 |
| M00001534A:C04 | 16921 | 4 | 0 | 0 | 1 | 2 | 1 |
| M00001534A:D09 | 5097 | 6 | 5 | 1 | 1 | 3 | 2 |
| M00001534A:F09 | 5321 | 11 | 7 | 1 | 5 | 10 | 26 |
| M00001534C:A01 | 4119 | 9 | 4 | 2 | 2 | 5 | 3 |
| M00001535A:B01 | 7665 | 3 | 1 | 5 | 0 | 0 | 0 |
| M00001535A:C06 | 20212 | 2 | 0 | 1 | 1 | 0 | 0 |
| M00001535A:F10 | 39423 | 2 | 0 | 0 | 0 | 0 | 0 |
| M00001536A:B07 | 2696 | 23 | 11 | 9 | 18 | 10 | 21 |
| M00001536A:C08 | 39392 | 2 | 0 | 0 | 0 | 0 | 0 |
| M00001537A:F12 | 39420 | 2 | 0 | 0 | 0 | 0 | 0 |
| M00001537B:G07 | 3389 | 4 | 11 | 13 | 2 | 0 | 0 |
| M00001540A:D06 | 8286 | 6 | 1 | 0 | 3 | 4 | 0 |
| M00001541A:D02 | 3765 | 19 | 6 | 0 | 0 | 0 | 0 |
| M00001541A:F07 | 22085 | 3 | 0 | 0 | 0 | 0 | 1 |
| M00001541A:H03 | 39174 | 2 | 0 | 0 | 0 | 0 | 0 |
| M00001542A:A09 | 22113 | 3 | 0 | 0 | 0 | 0 | 0 |
| M00001542A:E06 | 39453 | 2 | 0 | 0 | 0 | 0 | 0 |
| M00001544A:E03 | 12170 | 2 | 1 | 2 | 0 | 0 | 0 |
| M00001544A:G02 | 19829 | 2 | 0 | 1 | 0 | 0 | 0 |
| M00001544B:B07 | 6974 | 7 | 3 | 0 | 1 | 0 | 0 |
| M00001545A:C03 | 19255 | 2 | 0 | 0 | 0 | 0 | 0 |
| M00001545A:D08 | 13864 | 3 | 0 | 2 | 1 | 2 | 4 |
| M00001546A:G11 | 1267 | 43 | 55 | 5 | 0 | 0 | 0 |
| M00001548A:E10 | 5892 | 5 | 1 | 4 | 4 | 1 | 3 |
| M00001548A:H09 | 1058 | 40 | 44 | 37 | 47 | 39 | 59 |
| M00001549A:B02 | 4015 | 10 | 5 | 8 | 15 | 2 | 0 |
| M00001549A:D08 | 10944 | 3 | 0 | 3 | 1 | 0 | 7 |
| M00001549B:F06 | 4193 | 12 | 7 | 2 | 2 | 0 | 1 |
| M00001549C:E06 | 16347 | 4 | 0 | 0 | 0 | 0 | 0 |
| M00001550A:A03 | 7239 | 5 | 2 | 1 | 0 | 2 | 0 |
| M00001550A:G01 | 5175 | 8 | 1 | 3 | 2 | 0 | 0 |
| M00001551A:B10 | 6268 | 6 | 4 | 3 | 18 | 5 | 0 |
| M00001551A:F05 | 39180 | 2 | 0 | 0 | 0 | 0 | 0 |
| M00001551A:G06 | 22390 | 2 | 1 | 0 | 0 | 0 | 1 |
| M00001551C:G09 | 3266 | 12 | 14 | 0 | 1 | 0 | 6 |
| M00001552A:B12 | 307 | 73 | 60 | 196 | 75 | 79 | 27 |
| M00001552A:D11 | 39458 | 2 | 0 | 0 | 0 | 0 | 0 |
| M00001552B:D04 | 5708 | 5 | 4 | 4 | 3 | 1 | 4 |
| M00001553A:H06 | 8298 | 4 | 3 | 1 | 3 | 0 | 0 |
| M00001553B:F12 | 4573 | 5 | 7 | 2 | 5 | 0 | 1 |
| M00001553D:D10 | 22814 | 3 | 0 | 0 | 0 | 0 | 0 |
| M00001555A:B02 | 39539 | 2 | 0 | 0 | 0 | 1 | 0 |
| M00001555A:C01 | 39195 | 2 | 0 | 0 | 0 | 0 | 0 |
| M00001555D:G10 | 4561 | 8 | 4 | 4 | 8 | 0 | 0 |
| M00001556A:C09 | 9244 | 2 | 0 | 3 | 2 | 10 | 17 |
| M00001556A:F11 | 1577 | 12 | 40 | 25 | 3 | 4 | 0 |
| M00001556A:H01 | 15855 | 2 | 1 | 1 | 2 | 12 | 213 |
| M00001556A:C08 | 4386 | 7 | 8 | 3 | 1 | 3 | 21 |
| M00001556B:G02 | 11294 | 4 | 0 | 2 | 0 | 0 | 1 |
| M00001557A:D02 | 7065 | 5 | 3 | 2 | 1 | 0 | 0 |
| M00001557A:D02 | 7065 | 5 | 3 | 2 | 1 | 0 | 0 |
| M00001557A:F01 | 9635 | 3 | 0 | 2 | 1 | 0 | 0 |
| M00001557A:F03 | 39490 | 2 | 0 | 0 | 0 | 1 | 0 |
| M00001557B:H10 | 5192 | 8 | 5 | 0 | 5 | 0 | 0 |
| M00001557D:D09 | 8761 | 3 | 4 | 0 | 1 | 0 | 1 |
| M00001558B:H11 | 7514 | 5 | 3 | 0 | 0 | 0 | 0 |
| M00001560D:F10 | 6558 | 4 | 3 | 4 | 0 | 0 | 5 |
| M00001561A:C05 | 39486 | 2 | 0 | 0 | 0 | 0 | 0 |
| M00001563B:F06 | 102 | 289 | 233 | 278 | 116 | 123 | 184 |
| M00001564A:B12 | 5053 | 11 | 4 | 2 | 2 | 1 | 1 |
| M00001571C:H06 | 5749 | 4 | 1 | 9 | 0 | 0 | 0 |
| M00001578B:E04 | 23001 | 2 | 1 | 0 | 2 | 0 | 0 |
| M00001579D:C03 | 6539 | 8 | 3 | 0 | 0 | 0 | 1 |
| M00001583D:A10 | 6293 | 3 | 5 | 2 | 6 | 0 | 0 |
| M00001586C:C05 | 4623 | 3 | 4 | 12 | 2 | 1 | 1 |
| M00001587A:B11 | 39380 | 2 | 0 | 0 | 0 | 0 | 0 |
| M00001594B:H04 | 260 | 189 | 188 | 27 | 2 | 15 | 0 |
| M00001597C:H02 | 4837 | 6 | 2 | 10 | 0 | 3 | 1 |
| M00001597D:C05 | 10470 | 5 | 1 | 0 | 2 | 0 | 1 |
| M00001598A:G03 | 16999 | 4 | 0 | 0 | 0 | 0 | 0 |
| M00001601A:D08 | 22794 | 2 | 0 | 0 | 0 | 0 | 0 |
| M00001604A:B10 | 1399 | 49 | 27 | 19 | 7 | 10 | 23 |
| M00001604A:F05 | 39391 | 2 | 0 | 0 | 0 | 0 | 0 |
| M00001607A:E11 | 11465 | 5 | 0 | 0 | 0 | 0 | 0 |
| M00001608A:B03 | 7802 | 5 | 4 | 0 | 1 | 0 | 0 |
| M00001608B:E03 | 22155 | 3 | 0 | 0 | 0 | 0 | 0 |
| M00001614C:F10 | 13157 | 4 | 1 | 0 | 3 | 1 | 0 |
| M00001617C:E02 | 17004 | 4 | 0 | 1 | 0 | 1 | 0 |
| M00001619C:F12 | 40314 | 2 | 0 | 0 | 0 | 1 | 0 |
| M00001621C:C08 | 40044 | 2 | 0 | 0 | 0 | 0 | 0 |
| M00001623D:F10 | 13913 | 2 | 1 | 2 | 0 | 0 | 1 |
| M00001624A:B06 | 3277 | 10 | 11 | 8 | 3 | 5 | 1 |
| M00001624C:F01 | 4309 | 4 | 13 | 3 | 10 | 0 | 0 |
| M00001630B:H09 | 5214 | 10 | 2 | 2 | 2 | 4 | 3 |
| M00001644C:B07 | 39171 | 2 | 0 | 0 | 0 | 0 | 0 |
| M00001645A:C12 | 19267 | 2 | 0 | 0 | 0 | 0 | 1 |
| M00001648C:A01 | 4665 | 5 | 9 | 0 | 0 | 0 | 0 |
| M00001657D:C03 | 23201 | 3 | 0 | 0 | 0 | 3 | 0 |
| M00001657D:F08 | 76760 | 1 | 0 | 2 | 2 | 0 | 5 |
| M00001662C:A09 | 23218 | 3 | 0 | 0 | 0 | 0 | 0 |
| M00001663A:E04 | 35702 | 2 | 0 | 0 | 0 | 0 | 0 |
| M00001669B:F02 | 6468 | 4 | 3 | 3 | 8 | 1 | 0 |
| M00001670C:H02 | 14367 | 3 | 0 | 0 | 0 | 0 | 0 |
| M00001673C:H02 | 7015 | 6 | 3 | 1 | 2 | 1 | 1 |
| M00001675A:C09 | 8773 | 4 | 1 | 4 | 4 | 4 | 6 |
| M00001676B:F05 | 11460 | 4 | 2 | 0 | 0 | 0 | 0 |
| M00001677C:E10 | 14627 | 1 | 2 | 1 | 0 | 1 | 0 |
| M00001677D:A07 | 7570 | 5 | 3 | 0 | 0 | 0 | 0 |
| M00001678D:F12 | 4416 | 9 | 5 | 2 | 6 | 1 | 3 |
| M00001679A:A06 | 6660 | 7 | 0 | 4 | 2 | 1 | 0 |
| M00001679A:F10 | 26875 | 1 | 0 | 0 | 0 | 1 | 0 |
| M00001679B:F01 | 6298 | 2 | 4 | 5 | 3 | 1 | 0 |
| M00001679C:F01 | 78091 | 1 | 0 | 0 | 0 | 0 | 0 |
| M00001679D:D03 | 10751 | 3 | 2 | 0 | 1 | 0 | 1 |
| M00001679D:D03 | 10751 | 3 | 2 | 0 | 1 | 0 | 1 |
| M00001680D:F08 | 10539 | 2 | 1 | 1 | 0 | 1 | 0 |
| M00001682C:B12 | 17055 | 4 | 0 | 0 | 0 | 0 | 0 |
| M00001686A:E06 | 4622 | 7 | 6 | 4 | 2 | 3 | 0 |
| M00001688C:F09 | 5382 | 6 | 2 | 6 | 2 | 0 | 3 |
| M00001693C:G01 | 4393 | 10 | 6 | 2 | 4 | 1 | 1 |
| M00001716D:H05 | 67252 | 1 | 0 | 0 | 1 | 0 | 0 |
| M00003741D:C09 | 40108 | 2 | 0 | 0 | 0 | 0 | 0 |
| M00003747D:C05 | 11476 | 6 | 0 | 0 | 0 | 0 | 0 |
| M00003759B:B09 | 697 | 76 | 52 | 30 | 72 | 21 | 30 |
| M00003762C:B08 | 17076 | 4 | 0 | 0 | 0 | 0 | 0 |
| M00003763A:F06 | 3108 | 14 | 11 | 7 | 5 | 0 | 1 |
| M00003774C:A03 | 67907 | 1 | 0 | 0 | 0 | 0 | 0 |
| M00003796C:D05 | 5619 | 3 | 5 | 3 | 3 | 0 | 4 |
| M00003826B:A06 | 11350 | 3 | 3 | 0 | 0 | 1 | 0 |
| M00003833A:E05 | 21877 | 2 | 1 | 0 | 0 | 0 | 1 |
| M00003837D:A01 | 7899 | 5 | 4 | 0 | 2 | 1 | 0 |
| M00003839A:D08 | 7798 | 5 | 2 | 2 | 0 | 0 | 1 |
| M00003844C:B11 | 6539 | 8 | 3 | 0 | 0 | 0 | 1 |
| M00003846B:D06 | 6874 | 6 | 3 | 0 | 0 | 0 | 0 |
| M00003851B:D10 | 13595 | 4 | 0 | 1 | 0 | 0 | 1 |
| M00003853A:D04 | 5619 | 3 | 5 | 3 | 3 | 0 | 4 |
| M00003853A:F12 | 10515 | 5 | 1 | 0 | 1 | 1 | 2 |
| M00003856B:C02 | 4622 | 7 | 6 | 4 | 2 | 3 | 0 |
| M00003857A:G10 | 3389 | 4 | 11 | 13 | 2 | 0 | 0 |
| M00003857A:H03 | 4718 | 4 | 5 | 5 | 2 | 4 | 6 |
| M00003871C:E02 | 4573 | 5 | 7 | 2 | 5 | 0 | 1 |
| M00003875B:F04 | 12977 | 5 | 0 | 0 | 0 | 0 | 0 |
| M00003875B:F04 | 12977 | 5 | 0 | 0 | 0 | 0 | 0 |
| M00003875C:G07 | 8479 | 4 | 3 | 1 | 1 | 2 | 4 |
| M00003876D:E12 | 7798 | 5 | 2 | 2 | 0 | 0 | 1 |
| M00003879B:C11 | 5345 | 7 | 1 | 7 | 4 | 6 | 27 |
| M00003879B:D10 | 31587 | 1 | 1 | 0 | 0 | 1 | 0 |
| M00003879D:A02 | 14507 | 3 | 1 | 0 | 0 | 3 | 1 |
| M00003885C:A02 | 13576 | 5 | 0 | 0 | 0 | 12 | 11 |
| M00003885C:A02 | 13576 | 5 | 0 | 0 | 0 | 12 | 11 |
| M00003906C:E10 | 9285 | 4 | 3 | 0 | 0 | 1 | 2 |
| M00003907D:A09 | 39809 | 1 | 0 | 0 | 0 | 2 | 1 |
| M00003907D:H04 | 16317 | 3 | 0 | 0 | 0 | 0 | 0 |
| M00003909D:C03 | 8672 | 4 | 4 | 0 | 0 | 0 | 0 |
| M00003912B:D01 | 12532 | 4 | 1 | 0 | 1 | 0 | 1 |
| M00003914C:F05 | 3900 | 9 | 6 | 8 | 1 | 7 | 13 |
| M00003922A:E06 | 23255 | 3 | 0 | 1 | 1 | 0 | 0 |
| M00003958A:H02 | 18957 | 3 | 0 | 0 | 0 | 0 | 0 |
| M00003958A:H02 | 18957 | 3 | 0 | 0 | 0 | 0 | 0 |
| M00003958C:G10 | 40455 | 2 | 0 | 0 | 0 | 0 | 0 |
| M00003958C:G10 | 40455 | 2 | 0 | 0 | 0 | 0 | 0 |
| M00003968B:F06 | 24488 | 2 | 0 | 1 | 4 | 0 | 0 |
| M00003970C:B09 | 40122 | 2 | 0 | 0 | 0 | 0 | 0 |
| M00003974D:E07 | 23210 | 3 | 0 | 0 | 0 | 0 | 0 |
| M00003974D:H02 | 23358 | 3 | 0 | 0 | 0 | 1 | 0 |
| M00003975A:G11 | 12439 | 4 | 0 | 0 | 0 | 0 | 0 |
| M00003978B:G05 | 5693 | 7 | 4 | 1 | 3 | 1 | 1 |
| M00003981A:E10 | 3430 | 9 | 10 | 7 | 3 | 0 | 0 |
| M00003982C:C02 | 2433 | 10 | 13 | 21 | 18 | 8 | 8 |
| M00003983A:A05 | 9105 | 5 | 1 | 1 | 1 | 0 | 0 |
| M00004028D:A06 | 6124 | 4 | 8 | 1 | 9 | 1 | 0 |
| M00004028D:C05 | 40073 | 2 | 0 | 1 | 0 | 0 | 1 |
| M00004031A:A12 | 9061 | 5 | 2 | 0 | 0 | 0 | 0 |
| M00004031A:A12 | 9061 | 5 | 2 | 0 | 0 | 0 | 0 |
| M00004035C:A07 | 37285 | 2 | 0 | 0 | 1 | 0 | 1 |
| M00004035D:B06 | 17036 | 4 | 0 | 0 | 0 | 0 | 0 |
| M00004059A:D06 | 5417 | 10 | 4 | 0 | 9 | 2 | 0 |
| M00004068B:A01 | 3706 | 7 | 14 | 4 | 22 | 1 | 0 |
| M00004072B:B05 | 17036 | 4 | 0 | 0 | 0 | 0 | 0 |
| M00004081C:D10 | 15069 | 3 | 0 | 0 | 1 | 0 | 0 |
| M00004081C:D12 | 14391 | 3 | 1 | 0 | 0 | 0 | 0 |
| M00004086D:G06 | 9285 | 4 | 3 | 0 | 0 | 1 | 2 |
| M00004087D:A01 | 6880 | 2 | 6 | 1 | 1 | 0 | 0 |
| M00004093D:B12 | 5325 | 5 | 5 | 2 | 0 | 2 | 1 |
| M00004093D:B12 | 5325 | 5 | 5 | 2 | 0 | 2 | 1 |
| M00004105C:A04 | 7221 | 5 | 2 | 2 | 2 | 0 | 0 |
| M00004108A:E06 | 4937 | 4 | 9 | 3 | 1 | 3 | 1 |
| M00004111D:A08 | 6874 | 6 | 3 | 0 | 0 | 0 | 0 |
| M00004114C:F11 | 13183 | 2 | 3 | 0 | 7 | 0 | 1 |
| M00004138B:H02 | 13272 | 3 | 2 | 0 | 3 | 0 | 0 |
| M00004146C:C11 | 5257 | 2 | 8 | 5 | 5 | 5 | 25 |
| M00004151D:B08 | 16977 | 4 | 0 | 0 | 0 | 0 | 0 |
| M00004157C:A09 | 6455 | 3 | 1 | 6 | 0 | 0 | 0 |
| M00004169C:C12 | 5319 | 6 | 2 | 8 | 2 | 2 | 3 |
| M00004171D:B03 | 4908 | 6 | 7 | 2 | 2 | 2 | 0 |
| M00004172C:D08 | 11494 | 4 | 0 | 0 | 0 | 0 | 0 |
| M00004183C:D07 | 16392 | 3 | 0 | 0 | 0 | 0 | 0 |
| M00004185C:C03 | 11443 | 5 | 1 | 0 | 0 | 0 | 0 |
| M00004197D:H01 | 8210 | 2 | 6 | 0 | 0 | 0 | 0 |
| M00004203B:C12 | 14311 | 4 | 0 | 0 | 0 | 1 | 2 |
| M00004212B:C07 | 2379 | 26 | 13 | 4 | 2 | 2 | 3 |
| M00004214C:H05 | 11451 | 3 | 2 | 1 | 2 | 1 | 1 |
| M00004223A:G10 | 16918 | 4 | 0 | 0 | 0 | 0 | 0 |
| M00004223B:D09 | 7899 | 5 | 4 | 0 | 2 | 1 | 0 |
| M00004223D:E04 | 12971 | 4 | 0 | 0 | 0 | 1 | 0 |
| M00004229B:F08 | 6455 | 3 | 1 | 6 | 0 | 0 | 0 |
| M00004230B:C07 | 7212 | 3 | 5 | 2 | 1 | 3 | 0 |
| M00004269D:D06 | 4905 | 7 | 6 | 3 | 1 | 3 | 1 |
| M00004275C:C11 | 16914 | 3 | 0 | 0 | 1 | 0 | 0 |
| M00004283B:A04 | 14286 | 3 | 1 | 0 | 1 | 1 | 1 |
| M00004285B:E08 | 56020 | 1 | 0 | 0 | 0 | 0 | 0 |
| M00004295D:F12 | 16921 | 4 | 0 | 0 | 1 | 2 | 1 |
| M00004296C:H07 | 13046 | 4 | 1 | 0 | 1 | 0 | 0 |
| M00004307C:A06 | 9457 | 2 | 0 | 5 | 0 | 3 | 0 |
| M00004312A:G03 | 26295 | 2 | 0 | 0 | 0 | 0 | 0 |
| M00004318C:D10 | 21847 | 2 | 1 | 0 | 0 | 0 | 0 |
| M00004372A:A03 | 2030 | 13 | 10 | 32 | 4 | 0 | 0 |
| M00004377C:F05 | 2102 | 12 | 20 | 23 | 21 | 6 | 5 |

TABLE 6
All Differential Data for Libs 15-20

| | Cluster | Clones in | Clones in | Clones in | Clones in | Clones in | Clones in |
|---|---|---|---|---|---|---|---|
| Clone Name | ID | Lib15 | Lib16b | Lib17 | Lib18 | Lib19 | Lib20 |
| M00001340B:A06 | 17062 | 0 | 0 | 0 | 0 | 0 | 0 |
| M00001340D:F10 | 11589 | 0 | 0 | 0 | 0 | 0 | 0 |
| M00001341A:E12 | 4443 | 0 | 0 | 0 | 1 | 0 | 0 |
| M00001342B:E06 | 39805 | 0 | 0 | 0 | 0 | 0 | 0 |
| M00001343C:F10 | 2790 | 0 | 0 | 0 | 0 | 0 | 0 |
| M00001343D:H07 | 23255 | 0 | 0 | 0 | 0 | 0 | 0 |
| M00001345A:E01 | 6420 | 0 | 0 | 0 | 0 | 0 | 0 |
| M00001346A:F09 | 5007 | 0 | 0 | 0 | 0 | 0 | 0 |
| M00001346D:E03 | 6806 | 0 | 0 | 0 | 0 | 0 | 0 |
| M00001346D:G06 | 5779 | 0 | 0 | 0 | 0 | 0 | 0 |
| M00001346D:G06 | 5779 | 0 | 0 | 0 | 0 | 0 | 0 |
| M00001347A:B10 | 13576 | 0 | 0 | 0 | 0 | 0 | 0 |
| M00001348B:B04 | 16927 | 0 | 0 | 0 | 0 | 0 | 0 |
| M00001348B:G06 | 16985 | 0 | 0 | 0 | 0 | 0 | 0 |
| M00001349B:B08 | 3584 | 0 | 0 | 0 | 0 | 0 | 0 |
| M00001350A:H01 | 7187 | 0 | 0 | 0 | 0 | 0 | 0 |
| M00001351B:A08 | 3162 | 0 | 1 | 0 | 0 | 1 | 0 |
| M00001351B:A08 | 3162 | 0 | 1 | 0 | 0 | 1 | 0 |
| M00001352A:E02 | 16245 | 0 | 0 | 0 | 0 | 0 | 0 |
| M00001353A:G12 | 8078 | 0 | 0 | 0 | 0 | 0 | 0 |
| M00001353D:D10 | 14929 | 0 | 3 | 1 | 0 | 5 | 0 |
| M00001355B:G10 | 14391 | 0 | 0 | 0 | 0 | 0 | 0 |
| M00001357D:D11 | 4059 | 0 | 0 | 0 | 0 | 0 | 0 |
| M00001361A:A05 | 4141 | 0 | 0 | 0 | 0 | 0 | 0 |
| M00001361D:F08 | 2379 | 0 | 0 | 0 | 0 | 0 | 0 |
| M00001362B:D10 | 5622 | 0 | 0 | 0 | 0 | 0 | 0 |
| M00001362C:H11 | 945 | 0 | 0 | 0 | 0 | 0 | 1 |
| M00001365C:C10 | 40132 | 0 | 0 | 0 | 0 | 0 | 0 |
| M00001370A:C09 | 6867 | 0 | 0 | 0 | 0 | 0 | 0 |
| M00001371C:E09 | 7172 | 0 | 0 | 0 | 0 | 0 | 0 |
| M00001376B:G06 | 17732 | 0 | 0 | 0 | 0 | 0 | 1 |
| M00001378B:B02 | 39833 | 0 | 0 | 0 | 0 | 0 | 0 |
| M00001379A:A05 | 1334 | 0 | 0 | 0 | 0 | 0 | 1 |
| M00001380D:B09 | 39886 | 0 | 0 | 0 | 0 | 0 | 0 |
| M00001382C:A02 | 22979 | 0 | 0 | 0 | 0 | 0 | 0 |
| M00001383A:C03 | 39648 | 0 | 0 | 0 | 0 | 0 | 0 |
| M00001383A:C03 | 39648 | 0 | 0 | 0 | 0 | 0 | 0 |
| M00001386C:B12 | 5178 | 0 | 0 | 0 | 0 | 0 | 0 |
| M00001387A:C05 | 2464 | 0 | 0 | 0 | 0 | 0 | 0 |
| M00001387B:G03 | 7587 | 0 | 0 | 0 | 0 | 0 | 0 |
| M00001388D:G05 | 5832 | 0 | 0 | 0 | 0 | 0 | 0 |
| M00001389A:C08 | 16269 | 0 | 1 | 0 | 0 | 0 | 0 |
| M00001394A:F01 | 6583 | 1 | 4 | 1 | 0 | 0 | 0 |
| M00001395A:C03 | 4016 | 0 | 0 | 0 | 0 | 0 | 0 |
| M00001396A:C03 | 4009 | 0 | 0 | 0 | 0 | 0 | 0 |
| M00001402A:E08 | 39563 | 0 | 0 | 0 | 0 | 0 | 0 |
| M00001407B:D11 | 5556 | 0 | 0 | 0 | 0 | 0 | 0 |
| M00001409C:D12 | 9577 | 0 | 0 | 0 | 0 | 0 | 0 |
| M00001410A:D07 | 7005 | 0 | 0 | 0 | 0 | 0 | 0 |
| M00001412B:B10 | 8551 | 0 | 0 | 0 | 0 | 0 | 0 |
| M00001415A:H06 | 13538 | 0 | 0 | 0 | 0 | 0 | 0 |
| M00001416A:H01 | 7674 | 0 | 0 | 0 | 0 | 0 | 0 |
| M00001416B:H11 | 8847 | 0 | 0 | 0 | 0 | 0 | 0 |
| M00001417A:E02 | 36393 | 0 | 0 | 0 | 0 | 0 | 0 |
| M00001418B:F03 | 9952 | 0 | 0 | 0 | 0 | 0 | 0 |
| M00001418D:B06 | 8526 | 0 | 0 | 0 | 0 | 0 | 0 |
| M00001421C:F01 | 9577 | 0 | 0 | 0 | 0 | 0 | 0 |
| M00001423B:E07 | 15066 | 0 | 0 | 0 | 0 | 0 | 0 |
| M00001424B:G09 | 10470 | 0 | 0 | 0 | 0 | 0 | 0 |
| M00001425B:H08 | 22195 | 0 | 0 | 0 | 0 | 0 | 0 |
| M00001426D:C08 | 4261 | 0 | 0 | 1 | 0 | 0 | 1 |
| M00001428A:H10 | 84182 | 0 | 0 | 0 | 0 | 0 | 0 |
| M00001429A:H04 | 2797 | 0 | 0 | 0 | 0 | 0 | 0 |
| M00001429B:A11 | 4635 | 0 | 0 | 0 | 0 | 0 | 0 |
| M00001429D:D07 | 40392 | 0 | 0 | 0 | 0 | 0 | 0 |
| M00001439C:F08 | 40054 | 0 | 0 | 0 | 0 | 0 | 0 |
| M00001442C:D07 | 16731 | 0 | 0 | 0 | 0 | 0 | 0 |
| M00001445A:F05 | 13532 | 0 | 0 | 0 | 0 | 0 | 0 |
| M00001446A:F05 | 7801 | 0 | 0 | 0 | 0 | 0 | 0 |
| M00001447A:G03 | 10717 | 0 | 0 | 0 | 0 | 0 | 0 |
| M00001448D:C09 | 8 | 1 | 6 | 6 | 1 | 14 | 1 |
| M00001448D:H01 | 36313 | 0 | 3 | 0 | 0 | 3 | 0 |
| M00001449A:A12 | 5857 | 0 | 0 | 0 | 0 | 0 | 0 |
| M00001449A:B12 | 41633 | 0 | 0 | 0 | 0 | 0 | 0 |
| M00001449A:D12 | 3681 | 0 | 0 | 0 | 0 | 0 | 0 |
| M00001449A:G10 | 36535 | 0 | 0 | 0 | 0 | 0 | 0 |
| M00001449C:D06 | 86110 | 0 | 0 | 0 | 0 | 0 | 0 |
| M00001450A:A02 | 39304 | 0 | 0 | 0 | 0 | 0 | 0 |
| M00001450A:A11 | 32663 | 0 | 0 | 0 | 0 | 0 | 0 |
| M00001450A:B12 | 82498 | 0 | 0 | 0 | 0 | 0 | 0 |
| M00001450A:D08 | 27250 | 0 | 0 | 0 | 0 | 0 | 0 |
| M00001452A:B04 | 84328 | 0 | 0 | 0 | 0 | 0 | 0 |
| M00001452A:B12 | 86859 | 0 | 0 | 0 | 0 | 0 | 0 |
| M00001452A:D08 | 1120 | 0 | 0 | 0 | 0 | 0 | 0 |
| M00001452A:F05 | 85064 | 0 | 0 | 0 | 0 | 0 | 0 |
| M00001452C:B06 | 16970 | 0 | 0 | 2 | 0 | 1 | 0 |
| M00001453A:E11 | 16130 | 0 | 0 | 0 | 0 | 0 | 0 |
| M00001453C:F06 | 16653 | 0 | 0 | 0 | 0 | 0 | 0 |
| M00001454A:A09 | 83103 | 0 | 0 | 0 | 0 | 0 | 0 |
| M00001454B:C12 | 7005 | 0 | 0 | 0 | 0 | 0 | 0 |
| M00001454D:G03 | 689 | 0 | 2 | 2 | 0 | 4 | 2 |
| M00001455A:E09 | 13238 | 0 | 0 | 0 | 0 | 0 | 0 |
| M00001455B:E12 | 13072 | 0 | 0 | 0 | 0 | 0 | 0 |
| M00001455D:F09 | 9283 | 0 | 0 | 0 | 0 | 0 | 0 |
| M00001455D:F09 | 9283 | 0 | 0 | 0 | 0 | 0 | 0 |
| M00001460A:F06 | 2448 | 0 | 0 | 0 | 0 | 0 | 0 |
| M00001460A:F12 | 39498 | 0 | 0 | 0 | 0 | 0 | 0 |
| M00001461A:D06 | 1531 | 0 | 0 | 0 | 0 | 0 | 0 |
| M00001463C:B11 | 19 | 2 | 13 | 13 | 0 | 69 | 10 |
| M00001465A:B11 | 10145 | 0 | 0 | 0 | 0 | 0 | 0 |
| M00001466A:E07 | 4275 | 0 | 0 | 0 | 0 | 0 | 0 |
| M00001467A:B07 | 38759 | 0 | 0 | 0 | 0 | 0 | 0 |
| M00001467A:D04 | 39508 | 0 | 0 | 0 | 0 | 0 | 0 |
| M00001467A:D08 | 16283 | 0 | 0 | 0 | 0 | 0 | 0 |
| M00001467A:D08 | 16283 | 0 | 0 | 0 | 0 | 0 | 0 |
| M00001467A:E10 | 39442 | 0 | 0 | 0 | 0 | 0 | 0 |
| M00001468A:F05 | 7589 | 0 | 0 | 0 | 0 | 0 | 0 |
| M00001469A:C10 | 12081 | 0 | 0 | 0 | 0 | 0 | 0 |
| M00001469A:H12 | 19105 | 0 | 0 | 0 | 0 | 0 | 0 |
| M00001470A:B10 | 1037 | 0 | 0 | 0 | 0 | 0 | 0 |
| M00001470A:C04 | 39425 | 0 | 0 | 0 | 0 | 0 | 0 |
| M00001471A:B01 | 39478 | 0 | 0 | 0 | 0 | 0 | 0 |
| M00001481D:A05 | 7985 | 0 | 0 | 0 | 0 | 0 | 0 |
| M00001490B:C04 | 18699 | 0 | 0 | 0 | 0 | 0 | 0 |
| M00001494D:F06 | 7206 | 0 | 0 | 0 | 0 | 0 | 0 |
| M00001497A:G02 | 2623 | 0 | 0 | 0 | 0 | 0 | 0 |
| M00001499B:A11 | 10539 | 0 | 0 | 0 | 0 | 0 | 0 |
| M00001500A:C05 | 5336 | 0 | 0 | 0 | 0 | 0 | 0 |
| M00001500A:E11 | 2623 | 0 | 0 | 0 | 0 | 0 | 0 |
| M00001500C:E04 | 9443 | 0 | 0 | 0 | 0 | 0 | 0 |
| M00001501D:C02 | 9685 | 0 | 0 | 0 | 0 | 0 | 0 |
| M00001504C:A07 | 10185 | 0 | 0 | 0 | 0 | 0 | 0 |
| M00001504C:H06 | 6974 | 0 | 0 | 0 | 0 | 0 | 0 |
| M00001504D:G06 | 6420 | 0 | 0 | 0 | 0 | 0 | 0 |
| M00001507A:H05 | 39168 | 0 | 0 | 0 | 0 | 0 | 0 |
| M00001511A:H06 | 39412 | 0 | 0 | 0 | 0 | 0 | 0 |
| M00001512A:A09 | 39186 | 0 | 0 | 0 | 0 | 0 | 0 |
| M00001512D:G09 | 3956 | 0 | 0 | 1 | 0 | 0 | 0 |
| M00001513A:B06 | 4568 | 0 | 0 | 0 | 0 | 0 | 0 |
| M00001513C:E08 | 14364 | 0 | 0 | 0 | 0 | 0 | 0 |
| M00001514C:D11 | 40044 | 0 | 1 | 0 | 0 | 0 | 0 |
| M00001517A:B07 | 4313 | 0 | 0 | 0 | 0 | 0 | 0 |
| M00001518C:B11 | 8952 | 0 | 0 | 0 | 0 | 0 | 0 |
| M00001528A:C04 | 7337 | 0 | 0 | 0 | 0 | 0 | 0 |
| M00001528A:F09 | 18957 | 0 | 0 | 0 | 0 | 0 | 0 |
| M00001528B:H04 | 8358 | 0 | 0 | 0 | 0 | 0 | 0 |
| M00001531A:D01 | 38085 | 0 | 0 | 0 | 0 | 0 | 0 |
| M00001532B:A06 | 3990 | 1 | 1 | 0 | 0 | 0 | 0 |
| M00001533A:C11 | 2428 | 0 | 0 | 1 | 0 | 0 | 0 |
| M00001534A:C04 | 16921 | 0 | 0 | 0 | 0 | 0 | 0 |
| M00001534A:D09 | 5097 | 0 | 0 | 0 | 0 | 0 | 0 |
| M00001534A:F09 | 5321 | 0 | 1 | 0 | 0 | 2 | 0 |
| M00001534C:A01 | 4119 | 0 | 0 | 0 | 0 | 0 | 0 |
| M00001535A:B01 | 7665 | 0 | 0 | 0 | 0 | 0 | 0 |
| M00001535A:C06 | 20212 | 0 | 0 | 0 | 0 | 0 | 0 |
| M00001535A:F10 | 39423 | 0 | 0 | 0 | 0 | 0 | 0 |
| M00001536A:B07 | 2696 | 0 | 0 | 0 | 0 | 3 | 0 |
| M00001536A:C08 | 39392 | 0 | 0 | 0 | 0 | 0 | 0 |
| M00001537A:F12 | 39420 | 0 | 0 | 0 | 0 | 0 | 0 |
| M00001537B:G07 | 3389 | 0 | 0 | 0 | 0 | 0 | 0 |
| M00001540A:D06 | 8286 | 0 | 0 | 0 | 0 | 0 | 0 |
| M00001541A:D02 | 3765 | 0 | 0 | 0 | 0 | 0 | 0 |
| M00001541A:F07 | 22085 | 0 | 0 | 0 | 0 | 0 | 0 |
| M00001541A:H03 | 39174 | 0 | 0 | 0 | 0 | 0 | 0 |
| M00001542A:A09 | 22113 | 0 | 0 | 0 | 0 | 0 | 0 |
| M00001542A:E06 | 39453 | 0 | 0 | 0 | 0 | 0 | 0 |
| M00001544A:E03 | 12170 | 0 | 0 | 0 | 0 | 0 | 0 |
| M00001544A:G02 | 19829 | 0 | 0 | 0 | 0 | 0 | 0 |
| M00001544B:B07 | 6974 | 0 | 0 | 0 | 0 | 0 | 0 |
| M00001545A:C03 | 19255 | 0 | 0 | 0 | 0 | 0 | 0 |
| M00001545A:D08 | 13864 | 0 | 0 | 0 | 0 | 0 | 0 |
| M00001546A:G11 | 1267 | 1 | 0 | 0 | 0 | 7 | 0 |
| M00001548A:E10 | 5892 | 0 | 0 | 0 | 0 | 0 | 0 |
| M00001548A:H09 | 1058 | 0 | 0 | 1 | 0 | 0 | 0 |
| M00001549A:B02 | 4015 | 0 | 0 | 0 | 0 | 0 | 0 |
| M00001549A:D08 | 10944 | 0 | 0 | 0 | 0 | 0 | 0 |
| M00001549B:F06 | 4193 | 0 | 0 | 0 | 0 | 0 | 0 |
| M00001549C:E06 | 16347 | 0 | 0 | 0 | 0 | 0 | 0 |
| M00001550A:A03 | 7239 | 0 | 0 | 0 | 0 | 0 | 0 |
| M00001550A:G01 | 5175 | 0 | 0 | 0 | 0 | 0 | 0 |
| M00001551A:B10 | 6268 | 0 | 0 | 0 | 0 | 0 | 0 |
| M00001551A:F05 | 39180 | 0 | 0 | 0 | 0 | 0 | 0 |
| M00001551A:G06 | 22390 | 0 | 0 | 0 | 0 | 0 | 0 |
| M00001551C:G09 | 3266 | 0 | 0 | 1 | 0 | 0 | 0 |
| M00001552A:B12 | 307 | 0 | 0 | 0 | 0 | 3 | 0 |
| M00001552A:D11 | 39458 | 0 | 0 | 0 | 0 | 0 | 0 |
| M00001552B:D04 | 5708 | 0 | 1 | 0 | 0 | 0 | 0 |
| M00001553A:H06 | 8298 | 0 | 0 | 0 | 0 | 0 | 0 |
| M00001553B:F12 | 4573 | 0 | 0 | 0 | 0 | 0 | 0 |
| M00001553D:D10 | 22814 | 0 | 0 | 0 | 0 | 0 | 0 |
| M00001555A:B02 | 39539 | 0 | 0 | 0 | 0 | 0 | 0 |
| M00001555A:C01 | 39195 | 0 | 0 | 0 | 0 | 0 | 0 |
| M00001555D:G10 | 4561 | 0 | 0 | 0 | 0 | 0 | 0 |
| M00001556A:C09 | 9244 | 0 | 0 | 0 | 0 | 0 | 0 |
| M00001556A:F11 | 1577 | 0 | 0 | 0 | 0 | 0 | 0 |
| M00001556A:H01 | 15855 | 3 | 5 | 5 | 0 | 3 | 1 |
| M00001556B:C08 | 4386 | 1 | 2 | 0 | 0 | 0 | 0 |
| M00001556B:G02 | 11294 | 0 | 0 | 0 | 0 | 0 | 0 |
| M00001557A:D02 | 7065 | 0 | 0 | 0 | 0 | 0 | 0 |
| M00001557A:D02 | 7065 | 0 | 0 | 0 | 0 | 0 | 0 |
| M00001557A:F01 | 9635 | 0 | 0 | 0 | 0 | 0 | 0 |
| M00001557A:F03 | 39490 | 0 | 0 | 0 | 0 | 0 | 0 |
| M00001557B:H10 | 5192 | 0 | 0 | 0 | 0 | 0 | 0 |
| M00001557D:D09 | 8761 | 0 | 0 | 0 | 0 | 0 | 0 |
| M00001558B:H11 | 7514 | 0 | 0 | 0 | 0 | 0 | 0 |
| M00001560D:F10 | 6558 | 0 | 0 | 0 | 0 | 0 | 0 |
| M00001561A:C05 | 39486 | 0 | 0 | 0 | 0 | 0 | 0 |
| M00001563B:F06 | 102 | 22 | 38 | 65 | 7 | 43 | 10 |
| M00001564A:B12 | 5053 | 0 | 0 | 1 | 0 | 0 | 0 |
| M00001571C:H06 | 5749 | 0 | 0 | 0 | 0 | 0 | 0 |
| M00001578B:E04 | 23001 | 0 | 0 | 0 | 0 | 0 | 0 |
| M00001579D:C03 | 6539 | 0 | 0 | 0 | 0 | 0 | 0 |
| M00001583D:A10 | 6293 | 0 | 0 | 0 | 0 | 0 | 0 |
| M00001586C:C05 | 4623 | 0 | 0 | 0 | 0 | 1 | 0 |
| M00001587A:B11 | 39380 | 0 | 0 | 0 | 0 | 0 | 0 |
| M00001594B:H04 | 260 | 0 | 0 | 0 | 0 | 1 | 0 |
| M00001597C:H02 | 4837 | 0 | 0 | 0 | 0 | 0 | 0 |
| M00001597D:C05 | 10470 | 0 | 0 | 0 | 0 | 0 | 0 |
| M00001598A:G03 | 16999 | 1 | 1 | 1 | 0 | 0 | 0 |
| M00001601A:D08 | 22794 | 0 | 0 | 0 | 0 | 0 | 0 |
| M00001604A:B10 | 1399 | 0 | 0 | 0 | 0 | 0 | 0 |
| M00001604A:F05 | 39391 | 0 | 0 | 0 | 0 | 0 | 0 |
| M00001607A:E11 | 11465 | 0 | 0 | 0 | 0 | 0 | 0 |
| M00001608A:B03 | 7802 | 0 | 0 | 0 | 0 | 0 | 0 |
| M00001608B:E03 | 22155 | 0 | 0 | 0 | 0 | 0 | 0 |
| M00001614C:F10 | 13157 | 0 | 0 | 0 | 0 | 0 | 0 |
| M00001617C:E02 | 17004 | 0 | 0 | 0 | 0 | 1 | 0 |
| M00001619C:F12 | 40314 | 0 | 0 | 0 | 0 | 0 | 0 |
| M00001621C:C08 | 40044 | 0 | 1 | 0 | 0 | 0 | 0 |
| M00001623D:F10 | 13913 | 0 | 0 | 0 | 0 | 0 | 0 |
| M00001624A:B06 | 3277 | 0 | 0 | 0 | 0 | 0 | 0 |
| M00001624C:F01 | 4309 | 0 | 0 | 0 | 0 | 0 | 0 |
| M00001630B:H09 | 5214 | 1 | 0 | 0 | 1 | 1 | 0 |
| M00001644C:B07 | 39171 | 0 | 0 | 0 | 0 | 0 | 0 |
| M00001645A:C12 | 19267 | 0 | 0 | 0 | 0 | 1 | 0 |
| M00001648C:A01 | 4665 | 0 | 0 | 0 | 0 | 0 | 0 |
| M00001657D:C03 | 23201 | 0 | 0 | 0 | 0 | 0 | 0 |
| M00001657D:F08 | 76760 | 0 | 0 | 0 | 0 | 0 | 0 |
| M00001662C:A09 | 23218 | 0 | 0 | 0 | 0 | 0 | 0 |
| M00001663A:E04 | 35702 | 0 | 0 | 0 | 0 | 0 | 0 |
| M00001669B:F02 | 6468 | 0 | 0 | 0 | 0 | 0 | 0 |
| M00001670C:H02 | 14367 | 0 | 0 | 0 | 0 | 0 | 0 |
| M00001673C:H02 | 7015 | 0 | 0 | 0 | 0 | 0 | 0 |
| M00001675A:C09 | 8773 | 0 | 0 | 0 | 0 | 0 | 0 |
| M00001676B:F05 | 11460 | 0 | 0 | 0 | 0 | 0 | 0 |
| M00001677C:E10 | 14627 | 0 | 1 | 0 | 0 | 0 | 0 |
| M00001677D:A07 | 7570 | 0 | 0 | 0 | 0 | 0 | 0 |
| M00001678D:F12 | 4416 | 0 | 0 | 0 | 0 | 0 | 0 |
| M00001679A:A06 | 6660 | 0 | 0 | 0 | 0 | 0 | 0 |
| M00001679A:F10 | 26875 | 0 | 0 | 0 | 0 | 0 | 0 |
| M00001679B:F01 | 6298 | 0 | 0 | 0 | 0 | 0 | 0 |
| M00001679C:F01 | 78091 | 0 | 0 | 0 | 0 | 0 | 0 |
| M00001679D:D03 | 10751 | 0 | 0 | 0 | 0 | 0 | 0 |
| M00001679D:D03 | 10751 | 0 | 0 | 0 | 0 | 0 | 0 |
| M00001680D:F08 | 10539 | 0 | 0 | 0 | 0 | 0 | 0 |
| M00001682C:B12 | 17055 | 0 | 0 | 0 | 0 | 0 | 0 |
| M00001686A:E06 | 4622 | 0 | 0 | 0 | 0 | 0 | 0 |
| M00001688C:F09 | 5382 | 0 | 0 | 0 | 0 | 0 | 0 |
| M00001693C:G01 | 4393 | 0 | 0 | 0 | 0 | 0 | 0 |
| M00001716D:H05 | 67252 | 0 | 0 | 0 | 0 | 0 | 0 |
| M00003741D:C09 | 40108 | 0 | 0 | 0 | 0 | 0 | 0 |
| M00003747D:C05 | 11476 | 0 | 0 | 0 | 0 | 0 | 0 |
| M00003759B:B09 | 697 | 0 | 0 | 0 | 0 | 1 | 0 |
| M00003762C:B08 | 17076 | 0 | 0 | 0 | 0 | 0 | 0 |
| M00003763A:F06 | 3108 | 0 | 0 | 0 | 0 | 0 | 0 |
| M00003774C:A03 | 67907 | 0 | 0 | 0 | 0 | 0 | 0 |
| M00003796C:D05 | 5619 | 0 | 0 | 0 | 0 | 0 | 0 |
| M00003826B:A06 | 11350 | 0 | 0 | 0 | 0 | 0 | 0 |
| M00003833A:E05 | 21877 | 0 | 0 | 0 | 0 | 0 | 0 |
| M00003837D:A01 | 7899 | 0 | 0 | 0 | 0 | 0 | 0 |
| M00003839A:D08 | 7798 | 0 | 0 | 0 | 0 | 0 | 0 |
| M00003844C:B11 | 6539 | 0 | 0 | 0 | 0 | 0 | 0 |
| M00003846B:D06 | 6874 | 0 | 0 | 1 | 0 | 0 | 0 |
| M00003851B:D10 | 13595 | 0 | 0 | 0 | 0 | 0 | 0 |
| M00003853A:D04 | 5619 | 0 | 0 | 0 | 0 | 0 | 0 |
| M00003853A:F12 | 10515 | 0 | 0 | 0 | 0 | 0 | 0 |
| M00003856B:C02 | 4622 | 0 | 0 | 0 | 0 | 0 | 0 |
| M00003857A:G10 | 3389 | 0 | 0 | 0 | 0 | 0 | 0 |
| M00003857A:H03 | 4718 | 0 | 0 | 0 | 0 | 0 | 0 |
| M00003871C:E02 | 4573 | 0 | 0 | 0 | 0 | 0 | 0 |
| M00003875B:F04 | 12977 | 0 | 0 | 0 | 0 | 0 | 0 |
| M00003875B:F04 | 12977 | 0 | 0 | 0 | 0 | 0 | 0 |
| M00003875C:G07 | 8479 | 0 | 0 | 0 | 0 | 0 | 1 |
| M00003876D:E12 | 7798 | 0 | 0 | 0 | 0 | 0 | 0 |
| M00003879B:C11 | 5345 | 0 | 0 | 0 | 2 | 0 | 1 |
| M00003879B:D10 | 31587 | 0 | 0 | 0 | 0 | 0 | 0 |
| M00003879D:A02 | 14507 | 0 | 0 | 0 | 0 | 0 | 0 |
| M00003885C:A02 | 13576 | 0 | 0 | 0 | 0 | 0 | 0 |
| M00003885C:A02 | 13576 | 0 | 0 | 0 | 0 | 0 | 0 |
| M00003906C:E10 | 9285 | 0 | 0 | 0 | 0 | 0 | 0 |
| M00003907D:A09 | 39809 | 0 | 0 | 0 | 0 | 0 | 0 |
| M00003907D:H04 | 16317 | 0 | 0 | 0 | 0 | 0 | 0 |
| M00003909D:C03 | 8672 | 0 | 0 | 0 | 0 | 0 | 0 |
| M00003912B:D01 | 12532 | 0 | 0 | 0 | 0 | 0 | 0 |
| M00003914C:F05 | 3900 | 0 | 0 | 0 | 0 | 1 | 0 |
| M00003922A:E06 | 23255 | 0 | 0 | 0 | 0 | 0 | 0 |
| M00003958A:H02 | 18957 | 0 | 0 | 0 | 0 | 0 | 0 |
| M00003958A:H02 | 18957 | 0 | 0 | 0 | 0 | 0 | 0 |
| M00003958C:G10 | 40455 | 0 | 0 | 0 | 0 | 0 | 0 |
| M00003958C:G10 | 40455 | 0 | 0 | 0 | 0 | 0 | 0 |
| M00003968B:F06 | 24488 | 0 | 0 | 0 | 0 | 0 | 0 |
| M00003970C:B09 | 40122 | 0 | 0 | 0 | 0 | 0 | 0 |
| M00003974D:E07 | 23210 | 0 | 0 | 0 | 0 | 0 | 0 |
| M00003974D:H02 | 23358 | 0 | 0 | 0 | 0 | 0 | 0 |
| M00003975A:G11 | 12439 | 0 | 0 | 0 | 0 | 0 | 0 |
| M00003978B:G05 | 5693 | 0 | 0 | 0 | 0 | 0 | 0 |
| M00003981A:E10 | 3430 | 0 | 0 | 0 | 0 | 1 | 0 |
| M00003982C:C02 | 2433 | 0 | 0 | 0 | 0 | 0 | 0 |
| M00003983A:A05 | 9105 | 0 | 0 | 0 | 0 | 0 | 0 |
| M00004028D:A06 | 6124 | 0 | 0 | 0 | 0 | 0 | 0 |
| M00004028D:C05 | 40073 | 0 | 0 | 0 | 0 | 0 | 0 |
| M00004031A:A12 | 9061 | 0 | 0 | 0 | 0 | 0 | 0 |
| M00004031A:A12 | 9061 | 0 | 0 | 0 | 0 | 0 | 0 |
| M00004035C:A07 | 37285 | 0 | 0 | 0 | 0 | 0 | 0 |
| M00004035D:B06 | 17036 | 0 | 0 | 0 | 0 | 0 | 0 |
| M00004059A:D06 | 5417 | 0 | 0 | 0 | 0 | 0 | 0 |
| M00004068B:A01 | 3706 | 0 | 0 | 0 | 0 | 0 | 0 |
| M00004072B:B05 | 17036 | 0 | 0 | 0 | 0 | 0 | 0 |
| M00004081C:D10 | 15069 | 0 | 0 | 0 | 0 | 0 | 0 |
| M00004081C:D12 | 14391 | 0 | 0 | 0 | 0 | 0 | 0 |
| M00004086D:G06 | 9285 | 0 | 0 | 0 | 0 | 0 | 0 |
| M00004087D:A01 | 6880 | 0 | 0 | 0 | 0 | 0 | 0 |
| M00004093D:B12 | 5325 | 1 | 1 | 0 | 1 | 0 | 1 |
| M00004093D:B12 | 5325 | 1 | 1 | 0 | 1 | 0 | 1 |
| M00004105C:A04 | 7221 | 0 | 0 | 0 | 0 | 0 | 0 |
| M00004108A:E06 | 4937 | 0 | 0 | 0 | 0 | 0 | 0 |
| M00004111D:A08 | 6874 | 0 | 0 | 1 | 0 | 0 | 0 |
| M00004114C:F11 | 13183 | 0 | 0 | 0 | 0 | 0 | 0 |
| M00004138B:H02 | 13272 | 0 | 0 | 0 | 0 | 0 | 0 |
| M00004146C:C11 | 5257 | 0 | 1 | 0 | 0 | 0 | 0 |
| M00004151D:B08 | 16977 | 0 | 0 | 0 | 0 | 0 | 0 |
| M00004157C:A09 | 6455 | 0 | 0 | 0 | 0 | 0 | 0 |
| M00004169C:C12 | 5319 | 0 | 0 | 0 | 0 | 0 | 0 |
| M00004171D:B03 | 4908 | 0 | 0 | 0 | 0 | 0 | 0 |
| M00004172C:D08 | 11494 | 0 | 0 | 0 | 0 | 0 | 0 |
| M00004183C:D07 | 16392 | 0 | 0 | 0 | 0 | 0 | 0 |
| M00004185C:C03 | 11443 | 0 | 0 | 0 | 0 | 0 | 0 |
| M00004197D:H01 | 8210 | 0 | 0 | 0 | 0 | 0 | 0 |
| M00004203B:C12 | 14311 | 0 | 0 | 0 | 0 | 0 | 0 |
| M00004212B:C07 | 2379 | 0 | 0 | 0 | 0 | 0 | 0 |
| M00004214C:H05 | 11451 | 0 | 0 | 0 | 0 | 0 | 0 |
| M00004223A:G10 | 16918 | 0 | 0 | 0 | 0 | 0 | 0 |
| M00004223B:D09 | 7899 | 0 | 0 | 0 | 0 | 0 | 0 |
| M00004223D:E04 | 12971 | 0 | 0 | 0 | 0 | 0 | 0 |
| M00004229B:F08 | 6455 | 0 | 0 | 0 | 0 | 0 | 0 |
| M00004230B:C07 | 7212 | 0 | 0 | 0 | 0 | 0 | 0 |
| M00004269D:D06 | 4905 | 0 | 0 | 0 | 0 | 0 | 0 |
| M00004275C:C11 | 16914 | 0 | 0 | 0 | 0 | 0 | 0 |
| M00004283B:A04 | 14286 | 0 | 0 | 0 | 0 | 0 | 0 |
| M00004285B:E08 | 56020 | 0 | 0 | 0 | 0 | 0 | 0 |
| M00004295D:F12 | 16921 | 0 | 0 | 0 | 0 | 0 | 0 |
| M00004296C:H07 | 13046 | 0 | 0 | 0 | 0 | 0 | 0 |
| M00004307C:A06 | 9457 | 0 | 0 | 0 | 0 | 0 | 0 |
| M00004312A:G03 | 26295 | 0 | 0 | 0 | 0 | 0 | 0 |
| M00004318C:D10 | 21847 | 0 | 0 | 0 | 0 | 0 | 0 |
| M00004372A:A03 | 2030 | 0 | 0 | 0 | 0 | 0 | 0 |
| M00004377C:F05 | 2102 | 0 | 0 | 0 | 0 | 0 | 0 |

TABLE 7
All Differential Data for Libs 12-14

| | | Clones in | Clones in | Clones in |
|---|---|---|---|---|
| Clone Name | Cluster ID | Lib12 | Lib13 | Lib14 |
| M00001340B:A06 | 17062 | 0 | 0 | 0 |
| M00001340D:F10 | 11589 | 0 | 0 | 0 |
| M00001341A:E12 | 4443 | 4 | 2 | 0 |
| M00001342B:E06 | 39805 | 0 | 0 | 0 |
| M00001343C:F10 | 2790 | 0 | 0 | 0 |
| M00001343D:H07 | 23255 | 0 | 0 | 0 |
| M00001345A:E01 | 6420 | 0 | 0 | 0 |
| M00001346A:F09 | 5007 | 0 | 0 | 0 |
| M00001346D:E03 | 6806 | 0 | 1 | 1 |
| M00001346D:G06 | 5779 | 0 | 0 | 0 |
| M00001346D:G06 | 5779 | 0 | 0 | 0 |
| M00001347A:B10 | 13576 | 0 | 0 | 0 |
| M00001348B:B04 | 16927 | 0 | 0 | 0 |
| M00001348B:G06 | 16985 | 0 | 0 | 0 |
| M00001349B:B08 | 3584 | 0 | 0 | 0 |
| M00001350A:H01 | 7187 | 0 | 0 | 0 |
| M00001351B:A08 | 3162 | 0 | 0 | 1 |
| M00001351B:A08 | 3162 | 0 | 0 | 1 |
| M00001352A:E02 | 16245 | 0 | 0 | 0 |
| M00001353A:G12 | 8078 | 0 | 0 | 0 |
| M00001353D:D10 | 14929 | 0 | 1 | 0 |
| M00001355B:G10 | 14391 | 0 | 0 | 0 |
| M00001357D:D11 | 4059 | 0 | 0 | 0 |
| M00001361A:A05 | 4141 | 1 | 2 | 1 |
| M00001361D:F08 | 2379 | 0 | 0 | 0 |
| M00001362B:D10 | 5622 | 0 | 2 | 1 |
| M00001362C:H11 | 945 | 0 | 0 | 0 |
| M00001365C:C10 | 40132 | 0 | 0 | 0 |
| M00001370A:C09 | 6867 | 0 | 0 | 0 |
| M00001371C:E09 | 7172 | 0 | 0 | 1 |
| M00001376B:G06 | 17732 | 2 | 0 | 0 |
| M00001378B:B02 | 39833 | 0 | 0 | 0 |
| M00001379A:A05 | 1334 | 0 | 0 | 0 |
| M00001380D:B09 | 39886 | 0 | 0 | 0 |
| M00001382C:A02 | 22979 | 1 | 0 | 0 |
| M00001383A:C03 | 39648 | 0 | 0 | 0 |
| M00001383A:C03 | 39648 | 0 | 0 | 0 |
| M00001386C:B12 | 5178 | 0 | 0 | 0 |
| M00001387A:C05 | 2464 | 0 | 0 | 0 |
| M00001387B:G03 | 7587 | 0 | 0 | 0 |
| M00001388D:G05 | 5832 | 0 | 0 | 0 |
| M00001389A:C08 | 16269 | 2 | 0 | 0 |
| M00001394A:F01 | 6583 | 0 | 0 | 0 |
| M00001395A:C03 | 4016 | 0 | 0 | 0 |
| M00001396A:C03 | 4009 | 2 | 0 | 0 |
| M00001402A:E08 | 39563 | 0 | 0 | 0 |
| M00001407B:D11 | 5556 | 0 | 0 | 0 |
| M00001409C:D12 | 9577 | 0 | 0 | 0 |
| M00001410A:D07 | 7005 | 0 | 0 | 0 |
| M00001412B:B10 | 8551 | 0 | 0 | 0 |
| M00001415A:H06 | 13538 | 0 | 0 | 0 |
| M00001416A:H01 | 7674 | 0 | 0 | 0 |
| M00001416B:H11 | 8847 | 1 | 0 | 0 |
| M00001417A:E02 | 36393 | 0 | 0 | 0 |
| M00001418B:F03 | 9952 | 0 | 0 | 0 |
| M00001418D:B06 | 8526 | 0 | 0 | 0 |
| M00001421C:F01 | 9577 | 0 | 0 | 0 |
| M00001423B:E07 | 15066 | 0 | 0 | 0 |
| M00001424B:G09 | 10470 | 0 | 0 | 0 |
| M00001425B:H08 | 22195 | 0 | 0 | 0 |
| M00001426D:C08 | 4261 | 0 | 0 | 0 |
| M00001428A:H10 | 84182 | 0 | 0 | 0 |
| M00001429A:H04 | 2797 | 0 | 0 | 0 |
| M00001429B:A11 | 4635 | 0 | 0 | 0 |
| M00001429D:D07 | 40392 | 0 | 0 | 0 |
| M00001439C:F08 | 40054 | 0 | 0 | 0 |
| M00001442C:D07 | 16731 | 0 | 0 | 0 |
| M00001445A:F05 | 13532 | 0 | 0 | 0 |
| M00001446A:F05 | 7801 | 0 | 1 | 0 |
| M00001447A:G03 | 10717 | 0 | 0 | 0 |
| M00001448D:C09 | 8 | 7 | 6 | 9 |
| M00001448D:H01 | 36313 | 1 | 0 | 0 |
| M00001449A:A12 | 5857 | 0 | 0 | 0 |
| M00001449A:B12 | 41633 | 0 | 0 | 0 |
| M00001449A:D12 | 3681 | 1 | 0 | 0 |
| M00001449A:G10 | 36535 | 0 | 0 | 0 |
| M00001449C:D06 | 86110 | 0 | 0 | 0 |
| M00001450A:A02 | 39304 | 0 | 1 | 0 |
| M00001450A:A11 | 32663 | 0 | 0 | 0 |
| M00001450A:B12 | 82498 | 0 | 0 | 0 |
| M00001450A:D08 | 27250 | 0 | 0 | 0 |
| M00001452A:B04 | 84328 | 0 | 0 | 0 |
| M00001452A:B12 | 86859 | 0 | 0 | 0 |
| M00001452A:D08 | 1120 | 0 | 0 | 0 |
| M00001452A:F05 | 85064 | 0 | 0 | 0 |
| M00001452C:B06 | 16970 | 1 | 0 | 0 |
| M00001453A:E11 | 16130 | 0 | 0 | 0 |
| M00001453C:F06 | 16653 | 0 | 0 | 0 |
| M00001454A:A09 | 83103 | 0 | 0 | 0 |
| M00001454B:C12 | 7005 | 0 | 0 | 0 |
| M00001454D:G03 | 689 | 0 | 0 | 1 |
| M00001455A:E09 | 13238 | 0 | 0 | 0 |
| M00001455B:E12 | 13072 | 0 | 0 | 0 |
| M00001455D:F09 | 9283 | 0 | 0 | 0 |
| M00001455D:F09 | 9283 | 0 | 0 | 0 |
| M00001460A:F06 | 2448 | 0 | 0 | 0 |
| M00001460A:F12 | 39498 | 0 | 0 | 0 |
| M00001461A:D06 | 1531 | 0 | 0 | 1 |
| M00001463C:B11 | 19 | 17 | 32 | 31 |
| M00001465A:B11 | 10145 | 0 | 0 | 0 |
| M00001466A:E07 | 4275 | 0 | 0 | 0 |
| M00001467A:B07 | 38759 | 0 | 0 | 0 |
| M00001467A:D04 | 39508 | 0 | 0 | 0 |
| M00001467A:D08 | 16283 | 0 | 0 | 0 |
| M00001467A:D08 | 16283 | 0 | 0 | 0 |
| M00001467A:E10 | 39442 | 0 | 0 | 0 |
| M00001468A:F05 | 7589 | 0 | 0 | 0 |
| M00001469A:C10 | 12081 | 0 | 0 | 0 |
| M00001469A:H12 | 19105 | 0 | 0 | 0 |
| M00001470A:B10 | 1037 | 0 | 0 | 0 |
| M00001470A:C04 | 39425 | 0 | 0 | 0 |
| M00001471A:B01 | 39478 | 0 | 0 | 0 |
| M00001481D:A05 | 7985 | 0 | 0 | 0 |
| M00001490B:C04 | 18699 | 0 | 0 | 0 |
| M00001494D:F06 | 7206 | 0 | 0 | 0 |
| M00001497A:G02 | 2623 | 1 | 0 | 0 |
| M00001499B:A11 | 10539 | 0 | 1 | 0 |
| M00001500A:C05 | 5336 | 0 | 0 | 0 |
| M00001500A:E11 | 2623 | 1 | 0 | 0 |
| M00001500C:E04 | 9443 | 0 | 0 | 0 |
| M00001501D:C02 | 9685 | 0 | 0 | 0 |
| M00001504C:A07 | 10185 | 0 | 0 | 0 |
| M00001504C:H06 | 6974 | 0 | 0 | 0 |
| M00001504D:G06 | 6420 | 0 | 0 | 0 |
| M00001507A:H05 | 39168 | 0 | 0 | 0 |
| M00001511A:H06 | 39412 | 0 | 0 | 0 |
| M00001512A:A09 | 39186 | 0 | 0 | 0 |
| M00001512D:G09 | 3956 | 0 | 0 | 0 |
| M00001513A:B06 | 4568 | 0 | 0 | 0 |
| M00001513C:E08 | 14364 | 0 | 0 | 0 |
| M00001514C:D11 | 40044 | 0 | 0 | 0 |
| M00001517A:B07 | 4313 | 0 | 0 | 0 |
| M00001518C:B11 | 8952 | 0 | 0 | 0 |
| M00001528A:C04 | 7337 | 1 | 2 | 2 |
| M00001528A:F09 | 18957 | 0 | 0 | 0 |
| M00001528B:H04 | 8358 | 0 | 0 | 0 |
| M00001531A:D01 | 38085 | 0 | 0 | 0 |
| M00001532B:A06 | 3990 | 0 | 0 | 0 |
| M00001533A:C11 | 2428 | 0 | 0 | 0 |
| M00001534A:C04 | 16921 | 0 | 0 | 0 |
| M00001534A:D09 | 5097 | 0 | 0 | 0 |
| M00001534A:F09 | 5321 | 4 | 7 | 6 |
| M00001534C:A01 | 4119 | 0 | 0 | 0 |
| M00001535A:B01 | 7665 | 0 | 2 | 4 |
| M00001535A:C06 | 20212 | 0 | 0 | 0 |
| M00001535A:F10 | 39423 | 0 | 0 | 0 |
| M00001536A:B07 | 2696 | 0 | 0 | 0 |
| M00001536A:C08 | 39392 | 0 | 0 | 0 |
| M00001537A:F12 | 39420 | 0 | 0 | 0 |
| M00001537B:G07 | 3389 | 0 | 0 | 0 |
| M00001540A:D06 | 8286 | 0 | 0 | 0 |
| M00001541A:D02 | 3765 | 0 | 0 | 0 |
| M00001541A:F07 | 22085 | 0 | 0 | 0 |
| M00001541A:H03 | 39174 | 0 | 0 | 0 |
| M00001542A:A09 | 22113 | 0 | 0 | 0 |
| M00001542A:E06 | 39453 | 0 | 0 | 0 |
| M00001544A:E03 | 12170 | 0 | 0 | 0 |
| M00001544A:G02 | 19829 | 0 | 0 | 0 |
| M00001544B:B07 | 6974 | 0 | 0 | 0 |
| M00001545A:C03 | 19255 | 0 | 0 | 0 |
| M00001545A:D08 | 13864 | 0 | 0 | 0 |
| M00001546A:G11 | 1267 | 0 | 0 | 0 |
| M00001548A:E10 | 5892 | 0 | 1 | 0 |
| M00001548A:H09 | 1058 | 1 | 3 | 0 |
| M00001549A:B02 | 4015 | 0 | 1 | 0 |
| M00001549A:D08 | 10944 | 1 | 0 | 0 |
| M00001549B:F06 | 4193 | 0 | 0 | 0 |
| M00001549C:E06 | 16347 | 0 | 0 | 0 |
| M00001550A:A03 | 7239 | 0 | 1 | 0 |
| M00001550A:G01 | 5175 | 1 | 0 | 0 |
| M00001551A:B10 | 6268 | 0 | 0 | 1 |
| M00001551A:F05 | 39180 | 0 | 0 | 0 |
| M00001551A:G06 | 22390 | 0 | 0 | 1 |
| M00001551C:G09 | 3266 | 0 | 0 | 0 |
| M00001552A:B12 | 307 | 6 | 11 | 4 |
| M00001552A:D11 | 39458 | 0 | 0 | 0 |
| M00001552B:D04 | 5708 | 0 | 0 | 0 |
| M00001553A:H06 | 8298 | 0 | 0 | 0 |
| M00001553B:F12 | 4573 | 0 | 0 | 0 |
| M00001553D:D10 | 22814 | 0 | 0 | 0 |
| M00001555A:B02 | 39539 | 0 | 0 | 0 |
| M00001555A:C01 | 39195 | 0 | 0 | 0 |
| M00001555D:G10 | 4561 | 0 | 0 | 0 |
| M00001556A:C09 | 9244 | 0 | 1 | 0 |
| M00001556A:F11 | 1577 | 0 | 0 | 2 |
| M00001556A:H01 | 15855 | 1 | 1 | 0 |
| M00001556B:C08 | 4386 | 3 | 0 | 1 |
| M00001556B:G02 | 11294 | 0 | 0 | 0 |
| M00001557A:D02 | 7065 | 0 | 0 | 0 |
| M00001557A:D02 | 7065 | 0 | 0 | 0 |
| M00001557A:F01 | 9635 | 0 | 0 | 0 |
| M00001557A:F03 | 39490 | 0 | 0 | 0 |
| M00001557B:H10 | 5192 | 0 | 0 | 0 |
| M00001557D:D09 | 8761 | 0 | 0 | 0 |
| M00001558B:H11 | 7514 | 0 | 0 | 0 |
| M00001560D:F10 | 6558 | 0 | 0 | 0 |
| M00001561A:C05 | 39486 | 0 | 0 | 0 |
| M00001563B:F06 | 102 | 2 | 1 | 2 |
| M00001564A:B12 | 5053 | 0 | 0 | 0 |
| M00001571C:H06 | 5749 | 0 | 0 | 0 |
| M00001578B:E04 | 23001 | 0 | 0 | 0 |
| M00001579D:C03 | 6539 | 0 | 0 | 0 |
| M00001583D:A10 | 6293 | 0 | 0 | 0 |
| M00001586C:C05 | 4623 | 0 | 0 | 0 |
| M00001587A:B11 | 39380 | 0 | 0 | 0 |
| M00001594B:H04 | 260 | 1 | 0 | 0 |
| M00001597C:H02 | 4837 | 1 | 0 | 0 |
| M00001597D:C05 | 10470 | 0 | 0 | 0 |
| M00001598A:G03 | 16999 | 4 | 2 | 6 |
| M00001601A:D08 | 22794 | 0 | 0 | 0 |
| M00001604A:B10 | 1399 | 6 | 3 | 3 |
| M00001604A:F05 | 39391 | 0 | 0 | 0 |
| M00001607A:E11 | 11465 | 0 | 0 | 0 |
| M00001608A:B03 | 7802 | 0 | 0 | 0 |
| M00001608B:E03 | 22155 | 0 | 0 | 0 |
| M00001614C:F10 | 13157 | 0 | 0 | 0 |
| M00001617C:E02 | 17004 | 0 | 0 | 0 |
| M00001619C:F12 | 40314 | 0 | 0 | 0 |
| M00001621C:C08 | 40044 | 0 | 0 | 0 |
| M00001623D:F10 | 13913 | 0 | 0 | 0 |
| M00001624A:B06 | 3277 | 0 | 0 | 0 |
| M00001624C:F01 | 4309 | 0 | 0 | 0 |
| M00001630B:H09 | 5214 | 0 | 1 | 2 |
| M00001644C:B07 | 39171 | 0 | 0 | 0 |
| M00001645A:C12 | 19267 | 0 | 0 | 0 |
| M00001648C:A01 | 4665 | 0 | 0 | 0 |
| M00001657D:C03 | 23201 | 0 | 0 | 0 |
| M00001657D:F08 | 76760 | 0 | 0 | 0 |
| M00001662C:A09 | 23218 | 0 | 0 | 0 |
| M00001663A:E04 | 35702 | 0 | 0 | 0 |
| M00001669B:F02 | 6468 | 0 | 0 | 0 |
| M00001670C:H02 | 14367 | 0 | 0 | 0 |
| M00001673C:H02 | 7015 | 0 | 0 | 0 |
| M00001675A:C09 | 8773 | 0 | 0 | 0 |
| M00001676B:F05 | 11460 | 2 | 0 | 0 |
| M00001677C:E10 | 14627 | 0 | 0 | 0 |
| M00001677D:A07 | 7570 | 0 | 0 | 0 |
| M00001678D:F12 | 4416 | 1 | 2 | 0 |
| M00001679A:A06 | 6660 | 0 | 0 | 0 |
| M00001679A:F10 | 26875 | 0 | 0 | 0 |
| M00001679B:F01 | 6298 | 0 | 0 | 0 |
| M00001679C:F01 | 78091 | 0 | 0 | 0 |
| M00001679D:D03 | 10751 | 0 | 0 | 0 |
| M00001679D:D03 | 10751 | 0 | 0 | 0 |
| M00001680D:F08 | 10539 | 0 | 1 | 0 |
| M00001682C:B12 | 17055 | 0 | 0 | 0 |
| M00001686A:E06 | 4622 | 0 | 0 | 0 |
| M00001688C:F09 | 5382 | 0 | 0 | 0 |
| M00001693C:G01 | 4393 | 0 | 0 | 0 |
| M00001716D:H05 | 67252 | 0 | 0 | 0 |
| M00003741D:C09 | 40108 | 0 | 0 | 0 |
| M00003747D:C05 | 11476 | 0 | 0 | 0 |
| M00003759B:B09 | 697 | 0 | 0 | 0 |
| M00003762C:B08 | 17076 | 0 | 0 | 0 |
| M00003763A:F06 | 3108 | 0 | 0 | 0 |
| M00003774C:A03 | 67907 | 0 | 0 | 0 |
| M00003796C:D05 | 5619 | 0 | 1 | 0 |
| M00003826B:A06 | 11350 | 0 | 0 | 0 |
| M00003833A:E05 | 21877 | 0 | 0 | 0 |
| M00003837D:A01 | 7899 | 0 | 0 | 0 |
| M00003839A:D08 | 7798 | 0 | 0 | 0 |
| M00003844C:B11 | 6539 | 0 | 0 | 0 |
| M00003846B:D06 | 6874 | 0 | 0 | 0 |
| M00003851B:D10 | 13595 | 0 | 0 | 0 |
| M00003853A:D04 | 5619 | 0 | 1 | 0 |
| M00003853A:F12 | 10515 | 0 | 0 | 1 |
| M00003856B:C02 | 4622 | 0 | 0 | 0 |
| M00003857A:G10 | 3389 | 0 | 0 | 0 |
| M00003857A:H03 | 4718 | 0 | 0 | 0 |
| M00003871C:E02 | 4573 | 0 | 0 | 0 |
| M00003875B:F04 | 12977 | 0 | 0 | 0 |
| M00003875B:F04 | 12977 | 0 | 0 | 0 |
| M00003875C:G07 | 8479 | 1 | 0 | 0 |
| M00003876D:E12 | 7798 | 0 | 0 | 0 |
| M00003879B:C11 | 5345 | 4 | 8 | 3 |
| M00003879B:D10 | 31587 | 0 | 0 | 0 |
| M00003879D:A02 | 14507 | 0 | 0 | 0 |
| M00003885C:A02 | 13576 | 0 | 0 | 0 |
| M00003885C:A02 | 13576 | 0 | 0 | 0 |
| M00003906C:E10 | 9285 | 0 | 0 | 0 |
| M00003907D:A09 | 39809 | 0 | 0 | 0 |
| M00003907D:H04 | 16317 | 0 | 0 | 0 |
| M00003909D:C03 | 8672 | 0 | 0 | 0 |
| M00003912B:D01 | 12532 | 0 | 0 | 0 |
| M00003914C:F05 | 3900 | 0 | 1 | 0 |
| M00003922A:E06 | 23255 | 0 | 0 | 0 |
| M00003958A:H02 | 18957 | 0 | 0 | 0 |
| M00003958A:H02 | 18957 | 0 | 0 | 0 |
| M00003958C:G10 | 40455 | 0 | 0 | 0 |
| M00003958C:G10 | 40455 | 0 | 0 | 0 |
| M00003968B:F06 | 24488 | 0 | 0 | 0 |
| M00003970C:B09 | 40122 | 0 | 0 | 0 |
| M00003974D:E07 | 23210 | 0 | 0 | 0 |
| M00003974D:H02 | 23358 | 0 | 0 | 0 |
| M00003975A:G11 | 12439 | 0 | 0 | 0 |
| M00003978B:G05 | 5693 | 0 | 0 | 0 |
| M00003981A:E10 | 3430 | 0 | 0 | 0 |
| M00003982C:C02 | 2433 | 2 | 4 | 0 |
| M00003983A:A05 | 9105 | 0 | 0 | 0 |
| M00004028D:A06 | 6124 | 0 | 0 | 0 |
| M00004028D:C05 | 40073 | 0 | 1 | 0 |
| M00004031A:A12 | 9061 | 0 | 0 | 0 |
| M00004031A:A12 | 9061 | 0 | 0 | 0 |
| M00004035C:A07 | 37285 | 0 | 0 | 0 |
| M00004035D:B06 | 17036 | 0 | 0 | 0 |
| M00004059A:D06 | 5417 | 0 | 0 | 0 |
| M00004068B:A01 | 3706 | 0 | 0 | 0 |
| M00004072B:B05 | 17036 | 0 | 0 | 0 |
| M00004081C:D10 | 15069 | 0 | 0 | 0 |
| M00004081C:D12 | 14391 | 0 | 0 | 0 |
| M00004086D:G06 | 9285 | 0 | 0 | 0 |
| M00004087D:A01 | 6880 | 0 | 0 | 0 |
| M00004093D:B12 | 5325 | 0 | 0 | 0 |
| M00004093D:B12 | 5325 | 0 | 0 | 0 |
| M00004105C:A04 | 7221 | 0 | 0 | 0 |
| M00004108A:E06 | 4937 | 0 | 0 | 0 |
| M00004111D:A08 | 6874 | 0 | 0 | 0 |
| M00004114C:F11 | 13183 | 0 | 0 | 0 |
| M00004138B:H02 | 13272 | 0 | 0 | 0 |
| M00004146C:C11 | 5257 | 0 | 0 | 1 |
| M00004151D:B08 | 16977 | 0 | 0 | 0 |
| M00004157C:A09 | 6455 | 0 | 0 | 0 |
| M00004169C:C12 | 5319 | 0 | 0 | 0 |
| M00004171D:B03 | 4908 | 0 | 0 | 0 |
| M00004172C:D08 | 11494 | 0 | 0 | 0 |
| M00004183C:D07 | 16392 | 0 | 0 | 0 |
| M00004185C:C03 | 11443 | 2 | 0 | 0 |
| M00004197D:H01 | 8210 | 0 | 0 | 0 |
| M00004203B:C12 | 14311 | 0 | 0 | 0 |
| M00004212B:C07 | 2379 | 0 | 0 | 0 |
| M00004214C:H05 | 11451 | 0 | 0 | 0 |
| M00004223A:G10 | 16918 | 0 | 0 | 0 |
| M00004223B:D09 | 7899 | 0 | 0 | 0 |
| M00004223D:E04 | 12971 | 0 | 0 | 0 |
| M00004229B:F08 | 6455 | 0 | 0 | 0 |
| M00004230B:C07 | 7212 | 0 | 0 | 1 |
| M00004269D:D06 | 4905 | 0 | 0 | 0 |
| M00004275G:C11 | 16914 | 0 | 0 | 0 |
| M00004283B:A04 | 14286 | 0 | 0 | 0 |
| M00004285B:E08 | 56020 | 0 | 0 | 0 |
| M00004295D:F12 | 16921 | 0 | 0 | 0 |
| M00004296C:H07 | 13046 | 0 | 0 | 0 |
| M00004307C:A06 | 9457 | 1 | 0 | 0 |
| M00004312A:G03 | 26295 | 0 | 0 | 0 |
| M00004318C:D10 | 21847 | 0 | 0 | 0 |
| M00004372A:A03 | 2030 | 0 | 0 | 0 |
| M00004377C:F05 | 2102 | 0 | 0 | 0 |

We claim:

1. A library of polynucleotides, the library comprising the sequence information of at least one of SEQ ID NOS:1-844.

2. The library of claim 1, wherein the library is provided on a nucleic acid array.

3. The library of claim 1, wherein the library is provided in a computer-readable format.

4. The library of claim 1, wherein the library comprises a differentially expressed polynucleotide comprising a sequence selected from the group consisting of SEQ ID NOS:9, 39, 42, 52, 62, 74, 119, 172, 317, and 379.

5. The library of claim 1, wherein the library comprises a polynucleotide differentially expressed in a human breast cancer cell, where the polynucleotide comprises a sequence selected from the group consisting of SEQ ID NOS: 4, 9, 39, 42, 52, 62, 65, 66, 68, 74, 81, 114, 123, 144, 130, 157, 162, 172, 178, 183, 202, 214, 219, 223, 258, 298, 317, 338, 379, 384, 386, and 388.

6. The library of claim 1, wherein the library comprises a polynucleotide differentially expressed in a human colon cancer cell, where the polynucleotide comprises a sequence selected from the group consisting of SEQ ID NOS: 1, 39, 52, 97, 119, 134, 172, 176, 241, 288, 317, 357, 362, and 374.

7. The library of claim 1, wherein the library comprises a polynucleotide differentially expressed in a human lung cancer cell, where the polynucleotide comprises a sequence selected from the group consisting of SEQ ID NOS: 9, 34, 42, 62, 74, 106, 119, 135, 154, 160, 260, 308, 323, 349, 361, 369, 371, 379, 395, 381, and 400.

8. An isolated polynucleotide comprising a nucleotide sequence having at least 90% sequence identity to an identifying sequence of SEQ ID NOS:1-844 or a degenerate variant thereof.

9. An isolated polynucleotide according to claim 8, wherein the polynucleotide comprises a seqeuence encoding a polypeptide of a protein family selected from the group consisting of: 4 transmembrane segments integral membrane proteins, 7 transmembrane receptors, ATPases associated with various cellular activities (AAA), eukaryotic aspartyl proteases, GATA family of transcription factors, G-protein alpha subunit, phorbol esters/diacylglycerol binding proteins, protein kinase, protein phosphatase 2C, protein tyrosine phosphatase, trypsin, wnt family of developmental signaling proteins, and WW/rsp5/WWP domain containing proteins.

10. The polynucleotide of claim 9, wherein the polynucleotide comprises a sequence of one of SEQ ID NOS: 24, 41, 101, 157, 291, 305, 315, 341, 63, 116, 134, 136, 151, 384, 404, 308, 213, 367, 188, 251, 202, 315, 367, 397, 256, 382, 169, 23, 291, 324, 330, 341, 353, 188, 379, and 395.

11. The polynucleotide of claim 8, wherein the polynucleotide comprises a seqeuence encoding a polypeptide having a functional domain selected from the group consisting of: Ank repeat, basic region plus leucine zipper transcription factors, bromodomain, EF-hand, SH3 domain, WD domain/G-beta repeats, zinc finger (C2H2 type), zinc finger (CCHC class), and zinc-binding metalloprotease domain.

12. The polynucleotide of claim 11, wherein the polynucleotide comprises a sequence of one of SEQ ID NOS: 116, 251, 374, 97, 136, 242, 379, 306, 386, 18, 335, 61, 306, 386, 322, 306, and 395.

13. A recombinant host cell containing the polynucleotide of claim 8.

14. An isolated polypeptide encoded by the polynucleotide of claim 8.

15. An antibody that specifically binds a polypeptide of claim 14.

16. A vector comprising the polynucleotide of claim 8.

17. A polynucleotide comprising the nucleotide sequence of an insert contained in a clone deposited as ATCC accession number xx, xx, xx, xx, xx, xx, xx, xx, or xx.

18. A method of detecting differentially expressed genes correlated with a cancerous state of a mammalian cell, the method comprising the step of:
detecting at least one differentially expressed gene product in a test sample derived from a cell suspected of being cancerous, where the gene product is encoded by a gene corresponding to a sequence of at least one of SEQ ID NOS:4, 9, 39, 42, 52, 62, 65, 66, 68, 74, 81, 114, 123, 144, 130, 157, 162, 172, 178, 183, 202, 214, 219, 223, 258, 298, 317, 338, 379, 384, 386, 388, 1, 39, 52, 97, 119, 134, 172, 176, 241, 288, 317, 357, 362, 374, 9, 34, 42, 62, 74, 106, 119, 135, 154, 160, 260, 308, 323, 349, 361, 369, 371, 379, 395, 381, and 400;
wherein detection of the differentially expressed gene product is correlated with a cancerous state of the cell from which the test sample was derived.

19. The method of claim 18, wherein said detecting step is by hybridization of the test sample to a reference array, wherein the reference array comprises an identifying sequence of at least one of SEQ ID NOS:1-844.

20. The method of claim 18, wherein the cell is a breast tissue derived cell, and the differentially expressed gene product is encoded by a gene corresponding to a sequence of at least one of SEQ ID NOS: 4, 9, 39, 42, 52, 62, 65, 66, 68, 74, 81, 114, 123, 144, 130, 157, 162, 172, 178, 183, 202, 214, 219, 223, 258, 298, 317, 338, 379, 384, 386, and 388.

21. The method of claim 18, wherein the cell is a colon tissue derived cell, and the differentially expressed gene product is encoded by a gene corresponding to a sequence of at least one of SEQ ID NOS: 1, 39, 52, 97, 119, 134, 172, 176, 241, 288, 317, 357, 362, and 374.

22. The method of claim 18, wherein the cell is a lung tissue derived cell, and the differentially expressed gene product is encoded by a gene corresponding to a sequence of at least one of SEQ ID NOS: 9, 34, 42, 62, 74, 106, 119, 135, 154, 160, 260, 308, 323, 349, 361, 369, 371, 379, 395, 381, and 400.
